SEQUENCE LISTING (1 sequence)

SEQ ID NO: 1 - protein, 15 aa, synthetic construct
  REGION 1..15: note = Biotin-labeled peptide, custom synthesis
EQEDEPEGDYFEWLE

(19) United States
(12) Patent Application Publication
(10) Pub. No.: US 2024/0317744 A1
(43) Pub. Date: Sep. 26, 2024

(54) Bicyclic Ureas As Kinase Inhibitors

(71) Applicant: Incyte Corporation, Wilmington, DE (US)

(72) Inventors: Xin Li, Claymont, DE (US); Oleg Vechorkin, Wilmington, DE (US); Yanran Ai, West Chester, PA (US); Onur Atasoylu, Wilmington, DE (US); Hang Thi Dang, Wilmington, DE (US); Brent Douty, Fallowfield, PA (US); Chunhong He, Boothwyn, PA (US); Peng He, Wilmington, DE (US); Michael Liang, Wilmington, DE (US); Minh Nguyen, Claymont, DE (US); Evan Styduhar, Media, PA (US); Anlai Wang, Wilmington, DE (US); Xiaozhao Wang, Moorestown, NJ (US); Hai Fen Ye, Newark, DE (US); Eddy W. Yue, Landenberg, PA (US); Ke Zhang, Garnet Valley, PA (US); Peng Zhao, Newark, DE (US)

(21) Appl. No.: 18/602,434

(22) Filed: Mar. 12, 2024

Related U.S. Application Data
(60) Provisional application No. 63/598,333, filed on Nov. 13, 2023.
(60) Provisional application No. 63/588,817, filed on Oct. 9, 2023.
(60) Provisional application No. 63/451,867, filed on Mar. 13, 2023.

(51) Int. Cl. C07D 471/04; C07D 519/00

(52) U.S. Cl. CPC C07D 471/04; C07D 519/00

(57) ABSTRACT

The present application provides bicyclic urea compounds that modulate the activity of JAK2, which are useful in the treatment of various diseases, including cancer.

### SEQUENCE LISTING

This application contains a Sequence Listing that has been submitted electronically as an XML file named “20443-0806001_SL_ST26.XML.” The XML file, created on Mar. 12, 2024, is 2,281 bytes in size. The material in the XML file is hereby incorporated by reference in its entirety.

### TECHNICAL FIELD

The present invention provides bicyclic urea compounds that modulate the activity of the JAK2 and are useful in the treatment of diseases related to JAK2, including cancer.

### BACKGROUND

Janus kinase (JAK) 2 plays pivotal roles in signaling by several cytokine receptors. The mutant JAK2 V617F, located at pseudokinase (JH2) domain, is the most common molecular event associated with myeloproliferative neoplasms (MPNs). Current JAK2 small molecule inhibitors used to treat MPNs are designed to target the JAK2 kinase (JH1) domain. Thus, selective targeting of the JAK2 V617F mutant over the JAK2 kinase (JH1) domain may be useful for treating various pathologies, while sparing essential JAK2 functions. This application is directed to this need and others.

### SUMMARY

The present invention relates to, inter alia, compounds of Formula I:

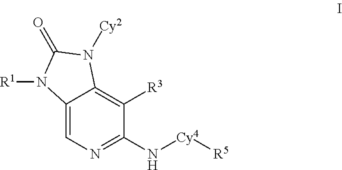

or pharmaceutically acceptable salts thereof, wherein constituent members are defined herein.

The present invention further provides pharmaceutical compositions comprising a compound of Formula I, or a pharmaceutically acceptable salt thereof, and a pharmaceutically acceptable carrier.

The present invention further provides methods of inhibiting an activity of the V617F variant of JAK2 kinase comprising contacting the kinase with a compound of Formula I, or a pharmaceutically acceptable salt thereof.

The present invention further provides methods of treating a disease or a disorder associated with expression or activity of the V617F variant of JAK2 kinase in a patient by administering to a patient a therapeutically effective amount of a compound of Formula I, or a pharmaceutically acceptable salt thereof.

The present invention further provides a compound of Formula I, or a pharmaceutically acceptable salt thereof, for use in any of the methods described herein.

The present invention further provides use of a compound of Formula I, or a pharmaceutically acceptable salt thereof, for the preparation of a medicament for use in any of the methods described herein.

### DETAILED DESCRIPTION

The present application provides a compound of Formula I:

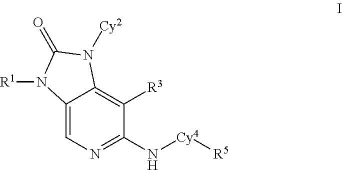

or a pharmaceutically acceptable salt thereof, wherein:
R¹ is selected from C₁₋₆ alkyl, C₂₋₆ alkenyl, and C₂₋₆ alkynyl, wherein the C₁₋₆ alkyl, C₂₋₆ alkenyl, and C₂₋₆ alkynyl of R¹ are each optionally substituted with 1, 2, or 3 independently selected R^{1A} substituents;
each R^{1A} is independently selected from halo, oxo, CN, NO₂, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{c11})R^{b11}, C(═NR^{c11})NR^{c11}R^{d11}, NR^{c11}C(═NR^{c11})NR^{c11}R^{d11}, NR^{c11}C(═NR^{c11})R^{b11}, NR^{c11}S(O)R^{b11}, NR^{c11}S(O)NR^{c11}R^{d11}, NR^{c11}S(O)₂R^{b11}, NR^{c11}S(O)(═NR^{c11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{c11})R^{b11}, and OS(O)₂R^{b11};
each R^{a11}, R^{b11}, R^{c11}, and R^{d11} is independently selected from H, C₁₋₆ alkyl, C₁₋₆ haloalkyl, C₂₋₆ alkenyl, and C₂₋₆ alkynyl, wherein the C₁₋₆ alkyl, C₂₋₆ alkenyl, and C₂₋₆ alkynyl of R^{a11}, R^{b11}, R^{c11} and R^{d11} are each optionally substituted with 1, 2, 3, 4, 5, 6, 7, or 8 independently selected R^{M} substituents;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, and C₂₋₆ alkynyl;

Cy² is selected from C₃₋₁₀ cycloalkyl and 4-12 membered heterocycloalkyl, wherein the C₃₋₁₀ cycloalkyl and 4-12 membered heterocycloalkyl are each substituted with 1, 2, 3, 4, 5, 6, 7 or 8 independently selected R² substituents;

each R² is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}C(═NR^{e2}) R^{b2}, NR^{c2}S(O)R^{b2}, NR^{c2}S(O)NR^{c2}R^{d2}, NR^{c2}S(O)₂R^{b2}, NR^{c2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, OS(O)₂R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, 4, 5, 6, 7, or 8 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a2}, R^{c2} and R^{d2} are each optionally substituted with 1, 2, 3, 4, 5, 6, 7, or 8 independently selected R^{2A} substituents;
or, any R^{c2} and R^{d2} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2A} substituents;
each R^{b2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b2} are each optionally substituted with 1, 2, 3, 4, 5, 6, 7, or 8 independently selected R^{2A} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f2} and R^{g2} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁-6 alkyl-;
each R^{h2} and Rⁱ² is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j2} and R^{k2} is independently selected from OH, C₁₋₆ alkoxy, and C₁₋₆ haloalkoxy;
or any R^{j2} and R^{k2} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a21}, SR^{a21}, NHOR^{a21}, C(O)R^{b21}, C(O)NR^{C21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}NR^{c21}R^{d21}, NR^{c21}C(O)R^{b21}, NR^{c21}C(O)OR^{a21}, NR^{c21}C(O)NR^{c21}R^{d21}, C(═NR^{e21})R^{b21}, C(═NR^{e21})NR^{c21}R^{d21}, NR^{c21}C(═NR^{e21})NR^{c21}R^{d21}, NR^{c21}C(═NR^{e21}) R^{b21}, NR^{c21}S(O)R^{b21}, NR^{c21}S(O)NR^{c21}R^{d21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, OS(O)₂R^{b21}, SF₅, P(O)R^{f21}R^{g21}, OP(O)(OR^{h21})(ORⁱ²¹), P(O)(OR^{h21})(ORⁱ²¹), and BR^{j21}R^{k21}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{2A} are each optionally substituted with 1, 2, 3, 4, 5, 6, 7, or 8 independently selected R^{2B} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a21}, R^{e21} and R^{d21} are each optionally substituted with 1, 2, 3, 4, 5, 6, 7, or 8 independently selected R^{2B} substituents;
or, any R^{c21} and R^{d21} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2B} substituents;
each R^{b21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b21} are each optionally substituted with 1, 2, 3, 4, 5, 6, 7, or 8 independently selected R^{2B} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁-6 alkyl-;
each R^{f21} and R^{g21} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁-6 alkyl-;
each R^{h21} and Rⁱ²¹ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j21} and R^{k21} is independently selected from OH, C₁₋₆ alkoxy, and C₁₋₆ haloalkoxy;
or any Rⁱ²¹ and R^{k21} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl; each R^{2B} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a22}, SR^{a22}, NHOR^{a22}, C(O)R^{b22}, C(O)NR^{c22}R^{d22}, C(O)NR^{c22}(OR^{a22}), C(O)OR^{a22}, OC(O)R^{b22}, OC(O)NR^{c22}R^{d22}, NR^{c22}R^{d22}, NR^{c22}NR^{c22}R^{d22}, NR^{c22}C(O)R^{b22}, NR^{c22}C(O)OR^{a22}, NR^{c22}C(O)NR^{c22}R^{d22}, C(═NR^{e22})R^{b22}, C(═NR^{e22})NR^{c22}R^{d22}, NR^{c22}C(═NR^{e22})NR^{c22}R^{d22}, NR^{c22}C(═NR^{e22}R^{d22}, NR^{c22}S(O)R^{b22}, NR^{c22}S(O)NR^{c22}R^{d22}, NR^{c22}S(O)₂R^{b22}, NR^{c22}S(O)(═NR^{e22})R^{b22}, NR^{c22}S(O)₂NR^{c22}R^{d22}, S(O)R^{b22}, S(O)NR^{c22}R^{d22}, S(O)₂R^{b22}, S(O)₂NR^{c22}R^{d22}, OS(O)(═NR^{e22}) R^{b22}, and OS(O)₂R^{b22}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl- of R^{2C} are each optionally substituted with 1, 2, 3, or 4 independently selected R^{M} substituents;
each R^{a22}, R^{c22}, and R^{d22} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁-6 alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a22}, R^{c22} and R^{d22} are each optionally substituted with 1, 2, 3, or 4 independently selected R^{M} substituents;
or, any R^{e22} and R^{d22} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b22} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b22} are each optionally substituted with 1, 2, 3, or 4 independently selected R^{M} substituents;
each R^{e22} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
R³ is selected from H, halo, C₁₋₆ alkyl, C₂₋₆ alkenyl, and C₂₋₆ alkynyl;
Cy⁴ is selected from C₆₋₁₀ aryl and 5-14 membered heteroaryl, wherein the C₆₋₁₀ aryl and 5-12 membered heteroaryl are each optionally substituted with 1, 2, 3, 4, 5, 6, 7 or 8 independently selected R⁴ substituents;
each R⁴ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a4}, SR^{a4}, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NR^{e4})NR^{c4}R^{d4}, NR^{c4}C(═NR^{e4})NR^{c4}R^{d4}, NR^{c4}C(═NR^{e4})R^{b4}, NR^{c4}S(O)R^{b4}, NR^{c4}S(O)NR^{c4}R^{d4}, NR^{c4}S(O)₂R^{b4}, NR^{c4}S(O)(═NR^{e4})R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, OS(O)(═NR^{e4})R^{b4}, OS(O)₂R^{b4}, SF₅, P(O)R^{f4}R^{g4}, OP(O)(OR^{h4})(ORⁱ⁴), P(O)(OR^{h4})(ORⁱ⁴), and BR^{j4}R^{k4}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R⁴ are each optionally substituted with 1, 2, 3, 4, 5, 6, 7, or 8 independently selected R^{4A} substituents;
each R^{a4}, R^{c4}, and R^{d4} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a4}, R^{c4} and R^{d4} are each optionally substituted with 1, 2, 3, 4, 5, 6, 7, or 8 independently selected R^{4A} substituents;
or, any R^{c4} and R^{d4} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{4A} substituents;
each R^{b4} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b4} are each optionally substituted with 1, 2, 3, 4, 5, 6, 7, or 8 independently selected R^{4A} substituents;
each R^{e4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f4} and R^{g4} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h4} and Rⁱ⁴ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j4} and R^{k4} is independently selected from OH, C₁₋₆ alkoxy, and C₁₋₆ haloalkoxy;
or any R^{j4} and R^{k4} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{4A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a41}, SR^{a41}, NHOR^{a41}, C(O)R^{b41}, C(O)NR^{c41}R^{d41}, C(O)NR^{c41}(OR^{a41}), C(O)OR^{a41}, OC(O)R^{b41} OC(O)NR^{c41}R^{d41}, NR^{c41}R^{d41}, NR^{c41}NR^{c41}R^{d41}, NR^{c41}C(O)R^{b41}, NR^{c41}C(O)OR^{a41}, NR^{c41}C(O)NR^{c41}R^{d41}, C(═NR^{e41})R^{b41}, C(═NR^{e41})NR^{c41}R^{d41}, NR^{c41}C(═NR^{e41})NR^{c41}R^{d41}, NR^{c41}C(═NR^{e41})R^{b41}, NR^{c41}S(O)R^{b41}, NR^{c41}S(O)NR^{c41}R^{d41}, NR^{c41}S(O)₂R^{b41}, NR^{c41}S(O)(═NR^{e41})R^{b41}, NR^{c41}S(O)₂NR^{c41}R^{d41}, S(O)R^{b41}, S(O)NR^{c41}R^{d41}, S(O)₂R^{b41}, S(O)₂NR^{c41}R^{d41}, OS(O)(═NR^{e41})R^{b41}, OS(O)₂R^{b41}, SF₅, P(O)R^{f41}R^{g41}, OP(O)(OR^{h41})(ORⁱ⁴¹), P(O)(OR^{h41})(ORⁱ⁴¹), and BR^{j41}R^{k41}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R⁴¹ are each optionally substituted with 1, 2, 3, 4, 5, 6, 7, or 8 independently selected R^{4B} substituents;
each R^{a41}, R^{c41}, and R^{d41} is independently selected from H, C₁₋₆ alkyl, C₁₋₆ haloalkyl,
C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a41}, R^{c41} and R^{d41} are each optionally substituted with 1, 2, 3, 4, 5, 6, 7, or 8 independently selected R^{4B} substituents;
or, any R^{c41} and R^{d41} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{4B} substituents;
each R^{b41} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b41} are each optionally substituted with 1, 2, 3, 4, 5, 6, 7, or 8 independently selected R^{4B} substituents;
each R^{e41} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f41} and R^{g41} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h41} and Rⁱ⁴¹ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j41} and R^{k41} is independently selected from OH, C₁₋₆ alkoxy, and C₁₋₆ haloalkoxy;
or any R^{j41} and R^{k41} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl; each R^{4B} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a42}, SR^{a42}, NHOR^{a42}, C(O)R^{b42}, C(O)NR^{c42}R^{d42}, C(O)NR^{c42}(OR^{a42}), C(O)OR^{a42}, OC(O)R^{b42}, OC(O)NR^{c42}R^{d42}, NR^{c42}R^{d42}, NR^{c42}NR^{c42}R^{d42}, NR^{c42}C(O)R^{b42}, NR^{c42}C(O)OR^{a42}, NR^{c42}C(O)NR^{c42}R^{d42}, C(═NR^{e42})R^{b42}, C(═NR^{e42})NR^{c42}R^{d42}, NR^{c42}C(═NR^{e42})NR^{c42}R^{d42}, NR^{c42}C(═NR^{e42})R^{b42}, NR^{c42}S(O)R^{b42}, NR^{c42}S(O)NR^{c42}R^{d42}, NR^{c42}S(O)₂R^{b42}, NR^{c42}S(O)(═NR^{e42}) R^{b42}, NR^{c42}S(O)₂NR^{c42}R^{d42}, S(O)R^{b42}, S(O)NR^{c42}R^{d42}, S(O)₂R^{b42}, S(O)₂NR^{c42}R^{d42}, OS(O)(═NR^{e42})R^{b42}, and OS(O)₂R^{b42}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁-6 alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{4C} are each optionally substituted with 1, 2, 3, or 4 independently selected R^{M} substituents;
each R^{a42}, R^{c42}, and R^{d42} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a42}, R^{c42} and R^{d42} are each optionally substituted with 1, 2, 3, or 4 independently selected R^{M} substituents;
or, any R^{c42} and R^{d42} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b42} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁-6 alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b42} are each optionally substituted with 1, 2, 3, or 4 independently selected R^{M} substituents;
each R^{e42} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
R⁵ is selected from C₁₋₆ alkyl, C₂₋₆ alkenyl, C₂₋₆ alkynyl, C₃₋₁₂ cycloalkyl, C₆₋₁₀ aryl, 5-12 membered heteroaryl, 4-12 membered heterocycloalkyl, OR^{w2}, SR^{w2}, NR^{w2}R^{w3}, C(O)R^{w2}, C(O)NR^{w2}R^{w3}, C(O)OR^{w2}, OC(O)R^{w2}, OC(NR^{w3})R^{w2}, OC(O)NR^{w2}R^{w3}, NR^{w3}C(O)R^{w2}, NR^{w3}C(O)OR^{w2}, NR^{w3}C(O)NR^{w2}R^{w3}, NR^{w3}S(O)R^{w2}, NR^{w3}S(O)NR^{w2}R^{w3}, NR 3S(O)₂R^{w2}, S(O)R^{w2}, S(O)NR^{w2}R^{w3}, S(O); R^{w2}, and S(O)₂NR^{w2}R^{w3};
wherein the C₁₋₆ alkyl and C₆₋₁₀ aryl of R⁵ are each substituted with 1 R^{w1} substituent and optionally substituted 1, 2, 3, 4, 5, 6, 7, or 8 independently selected R^{5A} substituents; or
the C₆₋₁₀ aryl of R⁵ is substituted with 1 R^{w5} substituent and optionally substituted 1, 2, 3, 4, 5, 6, 7, or 8 independently selected R^{5A} substituents; and
the C₂₋₆ alkenyl, C₂₋₆ alkynyl, C₃₋₁₂ cycloalkyl, 5-12 membered heteroaryl, and 4-12 membered heterocycloalkyl, of R⁵ are each optionally substituted with 1, 2, 3, 4, 5, 6, 7, or 8 independently selected R^{5A} substituents;
R^{w1} is selected from C₂₋₆ alkenyl, C₂₋₆ alkynyl, C₃₋₁₂ cycloalkyl, 5-12 membered heteroaryl, C₆₋₁₂ aryl, 4-12 membered heterocycloalkyl, OR^{w4}, NHR^{w4}, NR^{w3}R^{w4}, SR^{w4}, S(O)₂R^{w4}, C(O)R^{w4}, C(O)NHR^{w4}, C(O)NR^{w3}R^{w4}, OC(O)R^{w4}, S(O)R^{w3}, S(O)NR^{w3}R^{w3}, S(O)₂R^{w2}, and S(O)—NR^{w3}R³, wherein the C₂₋₆ alkenyl, C₂₋₆ alkynyl, C₃₋₁₂ cycloalkyl, 5-12 membered heteroaryl, C₆₋₁₂ aryl, and 4-12 membered heterocycloalkyl of R^{w1} are each optionally substituted with 1, 2, 3, 4, 5, 6, 7, or 8 independently selected R^{5A} substituents;
each R^{w2} is independently selected from C₃₋₁₂ cycloalkyl, 5-12 membered heteroaryl, and 4-12 membered heterocycloalkyl, wherein the C₃₋₁₂ cycloalkyl, 5-12 membered heteroaryl, and 4-12 membered heterocycloalkyl of R^{w2} are each optionally substituted with 1, 2, 3, 4, 5, 6, 7, or 8 independently selected R^{5A} substituents;
each R³ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{w3} are each optionally substituted with 1, 2, 3, 4, 5, 6, 7, or 8 independently selected R^{5A} substituents;
each R^{w4} is independently selected from C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a51}, R^{c51} and R^{d51} are each optionally substituted with 1, 2, 3, 4, 5, 6, 7, or 8 independently selected R^{5B} substituents;
R^{w5} is C₁₋₆ alkyl which is substituted with oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{c51}R^{d51}, NR^{c51}NR^{c51}R^{d51}, NR^{c51}C(O)R^{b51}, NR^{c51}C(O)OR^{a51}, NR^{c51}C(O)NR^{c51}R^{d51}, C(═NR^{e51})R^{b51}, C(═NR^{e51})NR^{c51}R^{d51}, NR^{c51}C(═NR^{e51})NR^{c51}R^{d51}, NR^{c51}C(═NR^{e51})R^{b51}, NR^{c51}S(O)R^{b51}, NR^{c51}S(O)NR^{c51}R^{d51}, NR^{c51}S(O)₂R^{b51}, NR^{c51}S(O)(═NR^{e51})R^{b51}, NR^{c51}S(O)₂NR^{c51}R^{d51}, S(O)R^{b51}, S(O)NR^{c51}R^{d51}, S(O)₂R^{b51}, S(O)₂NR^{c51}R^{d51}, OS(O)(═NR^{e51})R^{b51}, OS(O)₂R^{b51}, SF₅, P(O)R^{f51}R^{g51}, OP(O)(OR^{h51})(ORⁱ⁵¹), P(O)(OR^{h51})(ORⁱ⁵¹), or BR^{j51}R^{k51}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{w5} are each optionally substituted with 1, 2, 3, 4, 5, 6, 7, or 8 independently selected R^{5B} substituents;
each R^{5A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51} OC(O)NR^{c51}R^{d51}, NR^{c51}R^{d51}, NR^{c51}NR^{c51}R^{d51}, NR^{c51}C(O)R^{b51}, NR^{c51}C(O)OR^{a51}, NR^{c51}C(O)NR^{c51}R^{d51}, C(═NR^{e51})R^{b51}, C(═NR^{e51})NR^{c51}R^{d51}, NR^{c51}C(═NR^{e51})NR^{c51}R^{d51}, NR^{c51}C(═NR^{e51})R^{b51}, NR^{c51}S(O)R^{d51}, NR^{c51}S(O)NR^{c51}R^{d51}, NR^{c51}S(O)₂R^{b51}, NR^{c51}S(O)(═NR^{e51})R^{b51}, NR^{e51}S(O)₂NR^{e51}R^{d51}, S(O)R^{b51}, S(O)NR^{e51}R^{d51}, S(O)₂R^{b51}, S(O)₂NR^{c51}R^{d51}, OS(O)(═NR^{e51})R^{b51}, OS(O)₂R^{b51}, SF₅, P(O)R^{f51}R^{g51}, OP(O)(OR^{h51})(ORⁱ⁵¹), P(O)(OR^{h51})(ORⁱ⁵¹), and BRⁱ⁵¹R^{k51}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{5A} are each optionally substituted with 1, 2, 3, 4, 5, 6, 7, or 8 independently selected R^{5B} substituents;
each R^{a51}, R^{c51}, and R^{d51} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a51}, R^{e51} and R^{d51} are each optionally substituted with 1, 2, 3, 4, 5, 6, 7, or 8 independently selected R^{5B} substituents;
or, any R^{c51} and R^{d51} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{5B} substituents;
each R^{b51} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b51} are each optionally substituted with 1, 2, 3, 4, 5, 6, 7, or 8 independently selected R^{5B} substituents;
each R^{e51} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f51} and R^{g51} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁-6 alkyl-;
each R^{h51} and Rⁱ⁵¹ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j51} and R^{k51} is independently selected from OH, C₁₋₆ alkoxy, and C₁₋₆ haloalkoxy;
or any R^{j51} and R^{k51} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{5B} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a52}, SR^{a52}, NHOR^{a52}, C(O)R^{b52}, C(O)NR^{c52}R^{d52}, C(O)NR^{c52}(OR^{a52}), C(O)OR^{a52}, OC(O)R^{b52}, OC(O)NR^{c52}R^{d52}, NR^{c52}R^{d52}, NR^{c52}NR^{c52}R^{d52} NR^{c52}C(O)R^{b52}, NR^{c52}(O)OR^{a52}, NR^{c52}C(O)NR^{c52}R^{d52}, C(═NR^{e52}) R^{b52}, C(═NR^{e52})NR^{d52}R^{d52}, NR^{c52}C(═NR^{e52}) NR^{c52}R^{d52}, NR^{c52}C(═NR^{e52}) R^{b52}, NR^{c52}S(O)R^{b52}, NR^{c52}S(O)NR⁵²R^{d52}, NR⁵²S(O); R^{b52}, NR^{c52}S(O)(═NR^{e52}) R^{b52}, NR^{c52}S(O)₂NR^{c52}R^{d52}, S(O)R^{b52}, S(O)NR^{c52}R^{d52}, S(O); R^{b52}, S(O)₂NR^{c52}R^{d52} OS(O)(═NR^{e52})R^{b52}, and OS(O)₂R^{b52}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁-6 alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{5B} are each optionally substituted with 1, 2, 3, or 4 independently selected R^{M} substituents;
each R^{a52}, R^{c52}, and R^{d52} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a52}, R^{e52} and R^{d52} are each optionally substituted with 1, 2, 3, or 4 independently selected R^{M} substituents;
or, any R^{c52} and R^{d52} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b52} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b52} are each optionally substituted with 1, 2, 3, or 4 independently selected R^{M} substituents;
each R^{e52} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-; and
each R^{M} is independently selected from H, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
provided that;
(i) Cy⁴ is not triazolopyridinyl; and
(ii) R⁵ is not morpholinyl or piperazinyl; and
(iii) when Cy⁴ is phenyl, R⁵ is not pyridinyl or pyrimidinyl,

In some embodiments, Cy⁴ is selected from C₆₋₁₀ aryl and 5-14 membered heteroaryl, wherein the C₆₋₁₀ aryl and 5-12 membered heteroaryl of Cy⁴ are each optionally substituted with 1, 2, 3, or 4 independently selected R⁴ substituents;
provided that:
(i) Cy⁴ is not triazolopyridinyl; and
(ii) R⁵ is not morpholinyl or piperazinyl; and
(iii) when Cy⁴ is phenyl, R⁵ is not pyridinyl or pyrimidinyl.

In some embodiments, Cy⁴ is selected from phenyl and 5-14 membered heteroaryl, wherein the phenyl and 5-14 membered heteroaryl of Cy⁴ are each optionally substituted with 1, 2, 3, or 4 independently selected R⁴ substituents;
provided that:
(i) Cy⁴ is not triazolopyridinyl; and
(ii) R⁵ is not morpholinyl or piperazinyl; and
(iii) when Cy⁴ is phenyl, R⁵ is not pyridinyl or pyrimidinyl.

In some embodiments, Cy⁴ is selected from phenyl, monocyclic 5-6 membered heteroaryl, and bicyclic 8-14 membered heteroaryl, wherein the phenyl, monocyclic 5-6 membered heteroaryl, and bicyclic 8-14 membered heteroaryl of Cy⁴ are each optionally substituted with 1, 2, 3, or 4 independently selected R⁴ substituents;
provided that:
(i) Cy⁴ is not triazolopyridinyl; and
(ii) R⁵ is not morpholinyl or piperazinyl; and
(iii) when Cy⁴ is phenyl, R⁵ is not pyridinyl or pyrimidinyl.

In some embodiments, Cy⁴ is selected from phenyl, thiazolyl, pyrazolyl, pyridinyl, pyrimidinyl, quinolinyl, imidazo[1,2-b]pyridazinyl, pyrazolo[1,5-a]pyrimidinyl, pyrrolo[3,2-b]pyridinyl, 1,3-dihydro-2H-imidazo[4,5-b]pyridin-2-onyl, and pyrazolo[4,3-b]pyridinyl, wherein the phenyl, thiazolyl, pyrazolyl, pyridinyl, pyrimidinyl, quinolinyl, imidazo[1,2-b]pyridazinyl, pyrazolo[1,5-a]pyrimidinyl, pyrrolo[3,2-b]pyridinyl, 1,3-dihydro-2H-imidazo[4,5-b]pyridin-2-onyl, and pyrazolo[4,3-b]pyridinyl of Cy⁴ are each optionally substituted with 1, 2, 3, or 4 independently selected R⁴ substituents;
provided that:
(i) R⁵ is not morpholinyl or piperazinyl; and
(ii) when Cy⁴ is phenyl, R⁵ is not pyridinyl or pyrimidinyl.

In some embodiments, Cy⁴ is selected from phenyl, thiazolyl, pyrazolyl, pyridinyl, pyrimidinyl, quinolinyl, imidazo[1,2-b]pyridazinyl, and pyrazolo[1,5-a]pyrimidinyl, wherein the phenyl, thiazolyl, pyrazolyl, pyridinyl, pyrimidinyl, quinolinyl, imidazo[1,2-b]pyridazinyl, and pyrazolo[1,5-a]pyrimidinyl of Cy⁴ are each optionally substituted with 1, 2, 3, or 4 independently selected R⁴ substituents;
provided that:
(i) R⁵ is not morpholinyl or piperazinyl; and
(ii) when Cy⁴ is phenyl, R⁵ is not pyridinyl or pyrimidinyl.

In some embodiments, Cy⁴ is phenyl, which is optionally substituted with 1, 2, 3, or 4 independently selected R⁴ substituents, provided that R⁵ is not morpholinyl, piperazinyl, pyridinyl or pyrimidinyl.

In some embodiments, Cy⁴ is selected from thiazolyl, pyrazolyl, pyridinyl, pyrimidinyl, quinolinyl, imidazo[1,2-b]pyridazinyl, and pyrazolo[1,5-a]pyrimidinyl, wherein the thiazolyl, pyrazolyl, pyridinyl, pyrimidinyl, quinolinyl, imidazo[1,2-b]pyridazinyl, pyrazolo[1,5-a]pyrimidinyl, pyrrolo[3,2-b]pyridinyl, 1,3-dihydro-2H-imidazo[4,5-b]pyridin-2-onyl, and pyrazolo[4,3-b]pyridinyl of Cy⁴ are each optionally substituted with 1, 2, 3, or 4 independently selected R⁴ substituents, provided that R⁵ is not morpholinyl or piperazinyl.

In some embodiments, Cy⁴ is selected from thiazolyl, pyrazolyl, pyridinyl, pyrimidinyl, quinolinyl, imidazo[1,2-b]pyridazinyl, and pyrazolo[1,5-a]pyrimidinyl, wherein the thiazolyl, pyrazolyl, pyridinyl, pyrimidinyl, quinolinyl, imidazo[1,2-b]pyridazinyl, and pyrazolo[1,5-a]pyrimidinyl of Cy⁴ are each optionally substituted with 1, 2, 3, or 4 independently selected R⁴ substituents, provided that R⁵ is not morpholinyl or piperazinyl.

In some embodiments, Cy⁴ is selected from thiazolyl, pyrazolyl, pyridinyl, and pyrimidinyl, wherein the thiazolyl, pyrazolyl, pyridinyl, and pyrimidinyl of Cy⁴ are each optionally substituted with 1, 2, 3, or 4 independently selected R⁴ substituents, provided that R⁵ is not morpholinyl or piperazinyl.

In some embodiments, Cy⁴ is thiazolyl, which is optionally substituted with 1 or 2 independently selected R⁴ substituents, provided that R⁵ is not morpholinyl or piperazinyl.

In some embodiments, Cy⁴ is pyrazolyl, which is optionally substituted with 1 or 2 independently selected R⁴ substituents, provided that R⁵ is not morpholinyl or piperazinyl.

In some embodiments, Cy⁴ is pyridinyl, which is optionally substituted with 1, 2, 3, or 4 independently selected R⁴ substituents, provided that R⁵ is not morpholinyl or piperazinyl.

In some embodiments, Cy⁴ is pyrimidinyl, which is optionally substituted with 1, 2, 3, or 4 independently selected R⁴ substituents, provided that R⁵ is not morpholinyl or piperazinyl.

In some embodiments, Cy⁴ is selected from quinolinyl, imidazo[1,2-b]pyridazinyl, pyrazolo[1,5-a]pyrimidinyl, pyrrolo[3,2-b]pyridinyl, 1,3-dihydro-2H-imidazo[4,5-b]pyridin-2-onyl, and pyrazolo[4,3-b]pyridinyl, wherein the quinolinyl, imidazo[1,2-b]pyridazinyl, pyrazolo[1,5-a]pyrimidinyl, pyrrolo[3,2-b]pyridinyl, 1,3-dihydro-2H-imidazo[4,5-b]pyridin-2-onyl, and pyrazolo[4,3-b]pyridinyl of Cy⁴ are each optionally substituted with 1, 2, 3, or 4 independently selected R⁴ substituents, provided that R⁵ is not morpholinyl or piperazinyl.

In some embodiments, Cy⁴ is selected from quinolinyl, imidazo[1,2-b]pyridazinyl, and pyrazolo[1,5-a]pyrimidinyl, wherein the quinolinyl, imidazo[1,2-b]pyridazinyl, and pyrazolo[1,5-a]pyrimidinyl of Cy⁴ are each optionally substituted with 1, 2, 3, or 4 independently selected R⁴ substituents, provided that R⁵ is not morpholinyl or piperazinyl.

In some embodiments, Cy⁴ is quinolinyl, which is optionally substituted with 1, 2, 3, or 4 independently selected R⁴ substituents, provided that R⁵ is not morpholinyl or piperazinyl.

In some embodiments, Cy⁴ is imidazo[1,2-b]pyridazinyl, which is optionally substituted with 1, 2, 3, or 4 independently selected R⁴ substituents, provided that R⁵ is not morpholinyl or piperazinyl.

In some embodiments, Cy⁴ is pyrazolo[1,5-a]pyrimidinyl, which is optionally substituted with 1, 2, 3, or 4 independently selected R⁴ substituents, provided that R⁵ is not morpholinyl or piperazinyl.

In some embodiments, Cy⁴ is pyrrolo[3,2-b]pyridinyl, which is optionally substituted with 1, 2, 3, or 4 independently selected R⁴ substituents, provided that R⁵ is not morpholinyl or piperazinyl.

In some embodiments, Cy⁴ is 1,3-dihydro-2H-imidazo[4,5-b]pyridin-2-onyl, which is optionally substituted with 1, 2, 3, or 4 independently selected R⁴ substituents, provided that R⁵ is not morpholinyl or piperazinyl.

In some embodiments, Cy⁴ is pyrazolo[4,3-b]pyridinyl, which is optionally substituted with 1, 2, 3, or 4 independently selected R⁴ substituents, provided that R⁵ is not morpholinyl or piperazinyl.

In some embodiments, the compound of Formula I is a compound of Formula II:

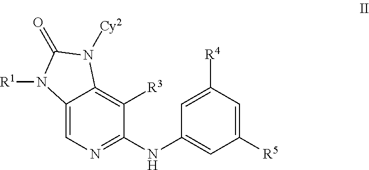

or a pharmaceutically acceptable salt thereof.

In some embodiments, the compound of Formula I is a compound of Formula IIa:

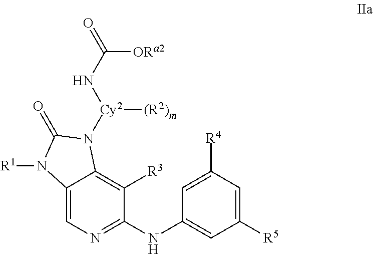

or a pharmaceutically acceptable salt thereof, wherein m is 0, 1, 2, 3, 4, 5, 6, or 7.

In some embodiments, the compound of Formula I is a compound of Formula IIb:

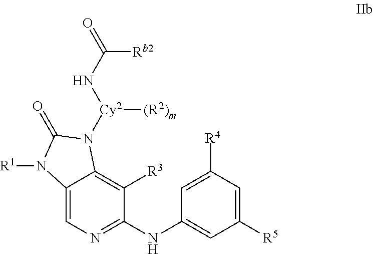

or a pharmaceutically acceptable salt thereof, wherein m is 0, 1, 2, 3, 4, 5, 6, or 7.

In some embodiments of Formulas IIa and IIb, m is 0, 1, 2, 3, or 4.

In some embodiments of Formulas IIa and IIb, m is 0, 1, 2, or 3.

In some embodiments of Formulas IIa and IIb, m is 0, 1, or 2.

In some embodiments of Formulas IIa and IIb, m is 0 or 1.

In some embodiments, the compound of Formula I is a compound of Formula IIIa, IIIb, IIIc, IIId, IIIe, IIIf, IIIg, IIIh, IIIi, IIIj, IIIk, IIIm, IIIn:

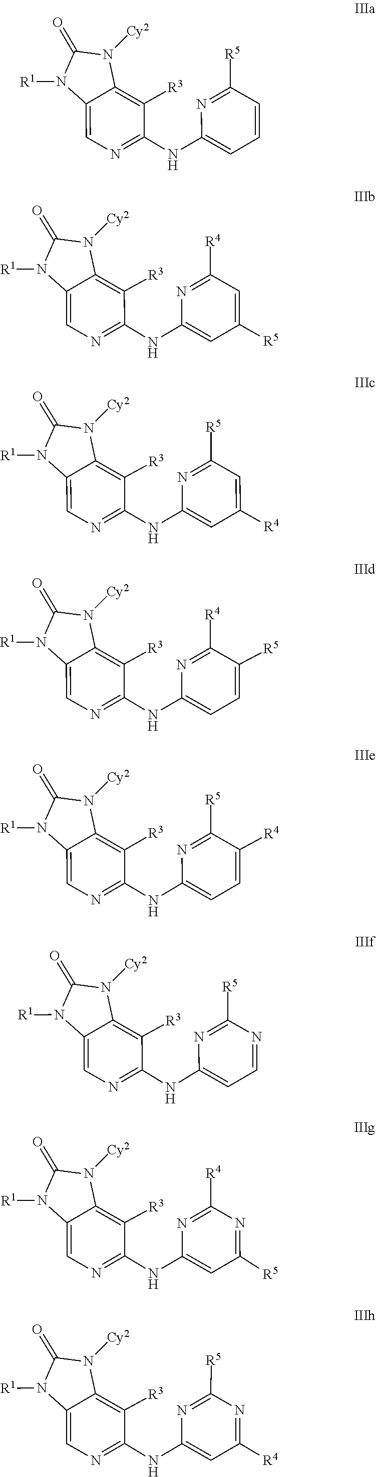

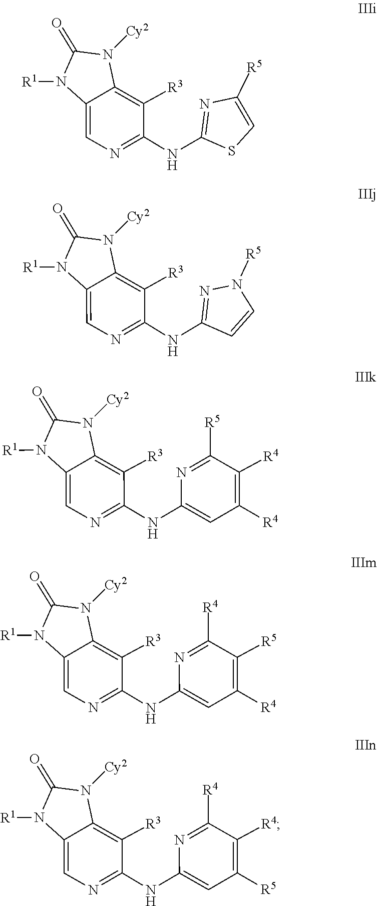

or a pharmaceutically acceptable salt thereof.

In some embodiments, the compound of Formula I is a compound of Formula IIIa, IIIb, IIIc, IIId, IIIe, IIIf, IIIg, IIIh, IIIi, or IIIj:

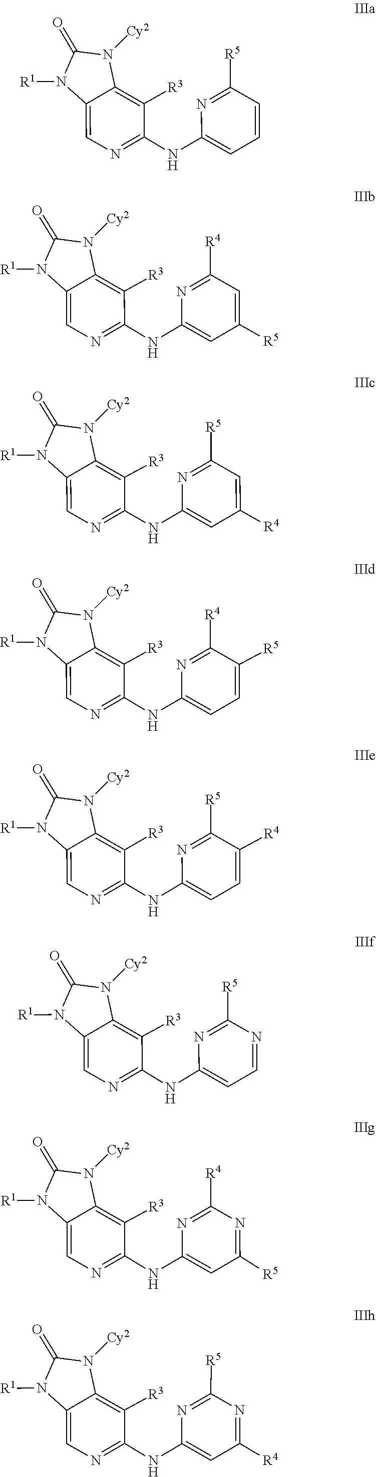

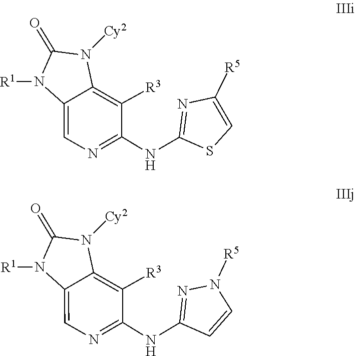

or a pharmaceutically acceptable salt thereof.

In some embodiments, the compound of Formula I is a compound of Formula IIIa, or a pharmaceutically acceptable salt thereof.

In some embodiments, the compound of Formula I is a compound of Formula IIIb, or a pharmaceutically acceptable salt thereof.

In some embodiments, the compound of Formula I is a compound of Formula IIIc, or a pharmaceutically acceptable salt thereof.

In some embodiments, the compound of Formula I is a compound of Formula IIId, or a pharmaceutically acceptable salt thereof.

In some embodiments, the compound of Formula I is a compound of Formula IIIe, or a pharmaceutically acceptable salt thereof.

In some embodiments, the compound of Formula I is a compound of Formula IIIf, or a pharmaceutically acceptable salt thereof.

In some embodiments, the compound of Formula I is a compound of Formula IIIg, or a pharmaceutically acceptable salt thereof.

In some embodiments, the compound of Formula I is a compound of Formula IIIh, or a pharmaceutically acceptable salt thereof.

In some embodiments, the compound of Formula I is a compound of Formula IIIi, or a pharmaceutically acceptable salt thereof.

In some embodiments, the compound of Formula I is a compound of Formula IIIj, or a pharmaceutically acceptable salt thereof.

In some embodiments, the compound of Formula I is a compound of Formula IIIk, or a pharmaceutically acceptable salt thereof.

In some embodiments, the compound of Formula I is a compound of Formula IIIm, or a pharmaceutically acceptable salt thereof.

In some embodiments, the compound of Formula I is a compound of Formula IIIn, or a pharmaceutically acceptable salt thereof.

In some embodiments, the compound of Formula I is a compound of Formula IVa, IVb, IVc, IVd, IVe, IVf, IVg, IVh, IVi, IVj, IVk, IVm, IVn, IVo, IVp, IVq, IVr, IVs, IVt, IVu, or IVv:

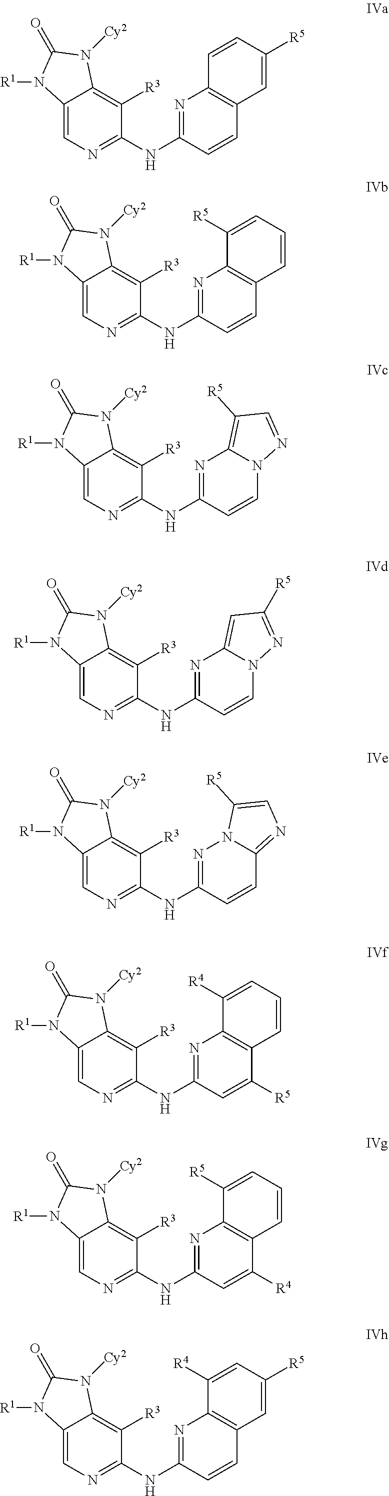

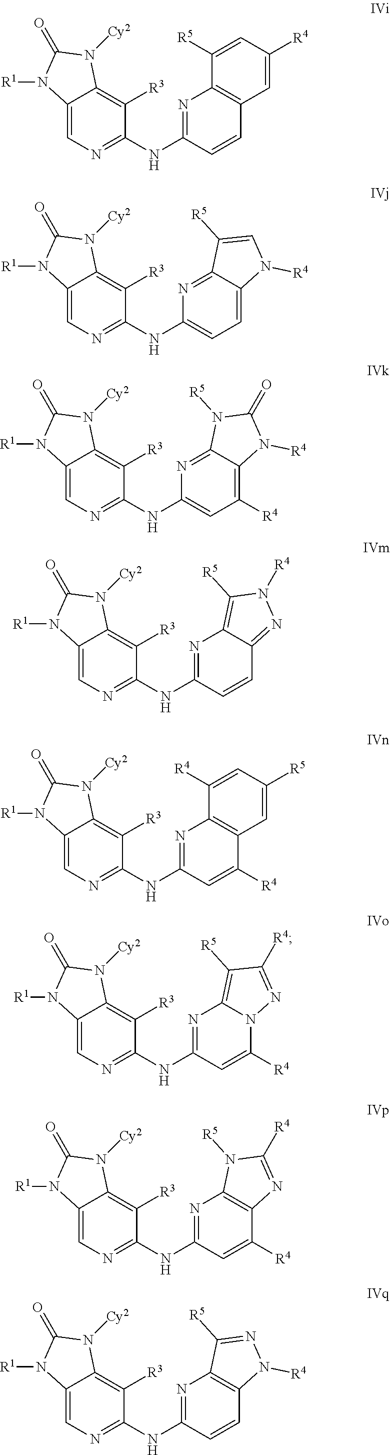

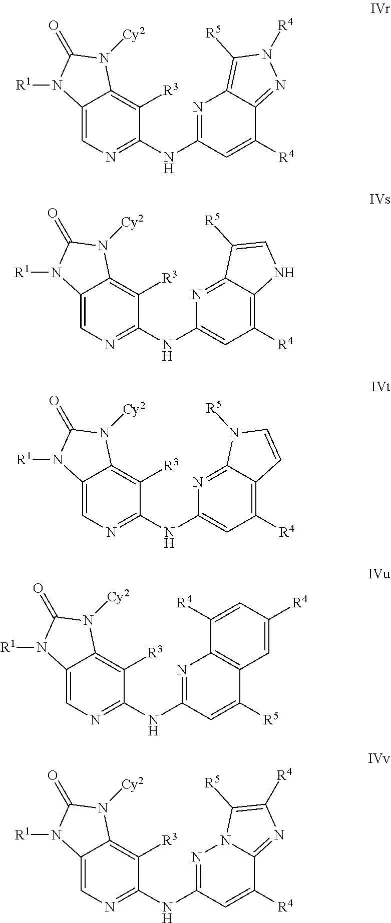

or a pharmaceutically acceptable salt thereof.

In some embodiments, the compound of Formula I is a compound of Formula IVa, IVb, IVc, IVd, IVe, IVf, IVg, IVh, IVi, IVj, IVk, IVm, IVn, IVo:

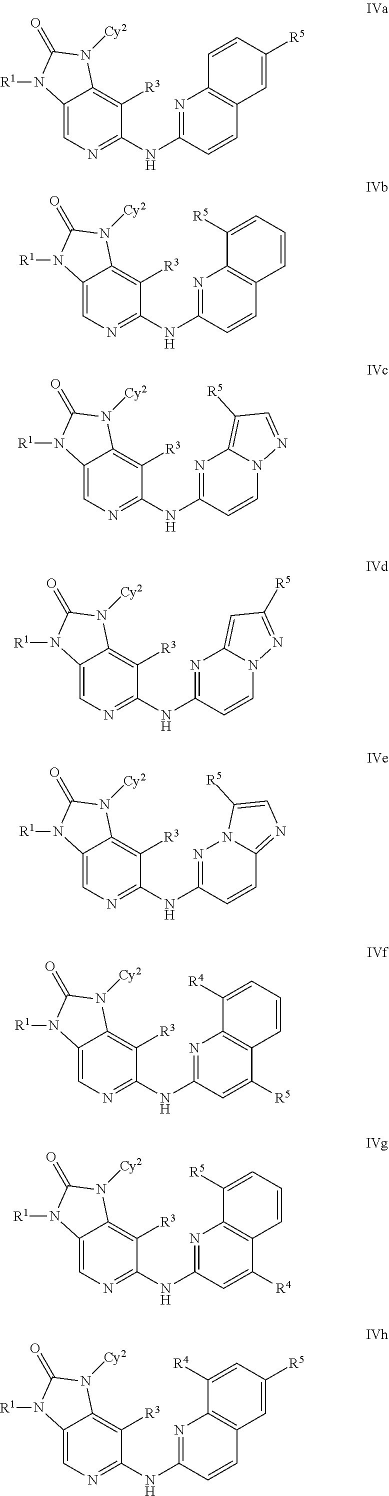

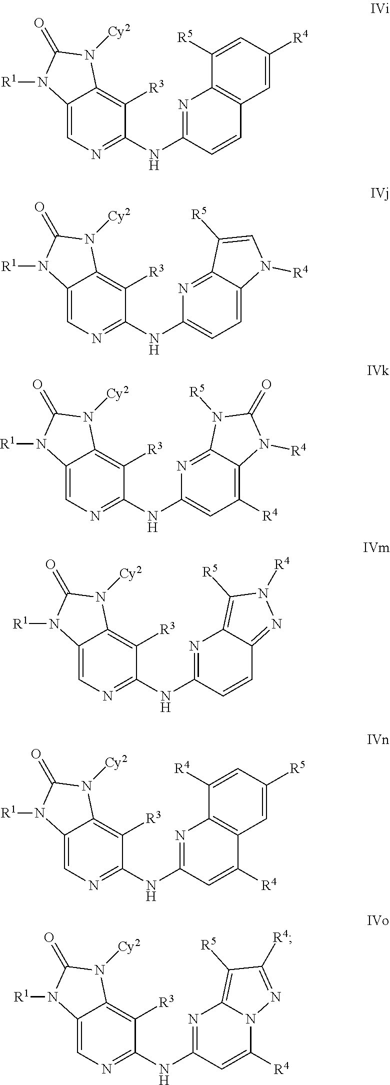

or a pharmaceutically acceptable salt thereof.

In some embodiments, the compound of Formula I is a compound of Formula IVa, IVb, IVc, IVd, IVe, IVf, IVg, IVh, or IVi:

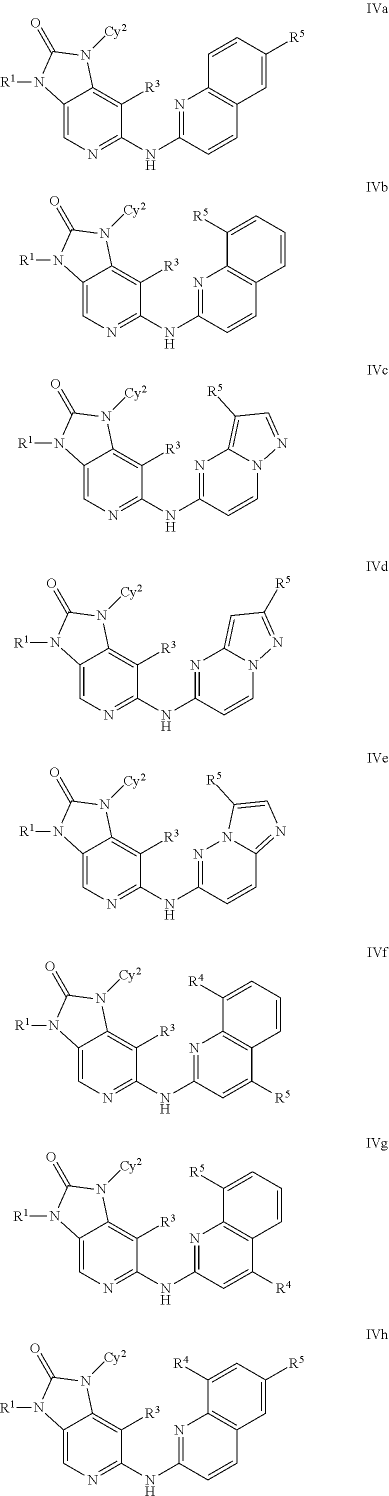

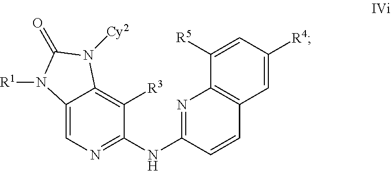

or a pharmaceutically acceptable salt thereof.

In some embodiments, the compound of Formula I is a compound of Formula IVa, or a pharmaceutically acceptable salt thereof.

In some embodiments, the compound of Formula I is a compound of Formula IVb, or a pharmaceutically acceptable salt thereof.

In some embodiments, the compound of Formula I is a compound of Formula IVc, or a pharmaceutically acceptable salt thereof.

In some embodiments, the compound of Formula I is a compound of Formula IVd, or a pharmaceutically acceptable salt thereof.

In some embodiments, the compound of Formula I is a compound of Formula IVe, or a pharmaceutically acceptable salt thereof.

In some embodiments, the compound of Formula I is a compound of Formula IVf, or a pharmaceutically acceptable salt thereof.

In some embodiments, the compound of Formula I is a compound of Formula IVg, or a pharmaceutically acceptable salt thereof.

In some embodiments, the compound of Formula I is a compound of Formula IVh, or a pharmaceutically acceptable salt thereof.

In some embodiments, the compound of Formula I is a compound of Formula IVi, or a pharmaceutically acceptable salt thereof.

In some embodiments, the compound of Formula I is a compound of Formula IVj, or a pharmaceutically acceptable salt thereof.

In some embodiments, the compound of Formula I is a compound of Formula IVk, or a pharmaceutically acceptable salt thereof.

In some embodiments, the compound of Formula I is a compound of Formula IVm, or a pharmaceutically acceptable salt thereof.

In some embodiments, the compound of Formula I is a compound of Formula IVn, or a pharmaceutically acceptable salt thereof.

In some embodiments, the compound of Formula I is a compound of Formula IVo, or a pharmaceutically acceptable salt thereof.

In some embodiments, the compound of Formula I is a compound of Formula IVp, or a pharmaceutically acceptable salt thereof.

In some embodiments, the compound of Formula I is a compound of Formula IVq, or a pharmaceutically acceptable salt thereof.

In some embodiments, the compound of Formula I is a compound of Formula IVr, or a pharmaceutically acceptable salt thereof.

In some embodiments, the compound of Formula I is a compound of Formula IVs, or a pharmaceutically acceptable salt thereof.

In some embodiments, the compound of Formula I is a compound of Formula IVt, or a pharmaceutically acceptable salt thereof.

In some embodiments, the compound of Formula I is a compound of Formula IVu, or a pharmaceutically acceptable salt thereof.

In some embodiments, the compound of Formula I is a compound of Formula IVv, or a pharmaceutically acceptable salt thereof.

In some embodiments, the compound of Formula I is a compound of Formula Va, Vb, Vc, Vd, Vc, Vf, Vg, Vh, VIi, Vj, Vk, Vm, or Vn:

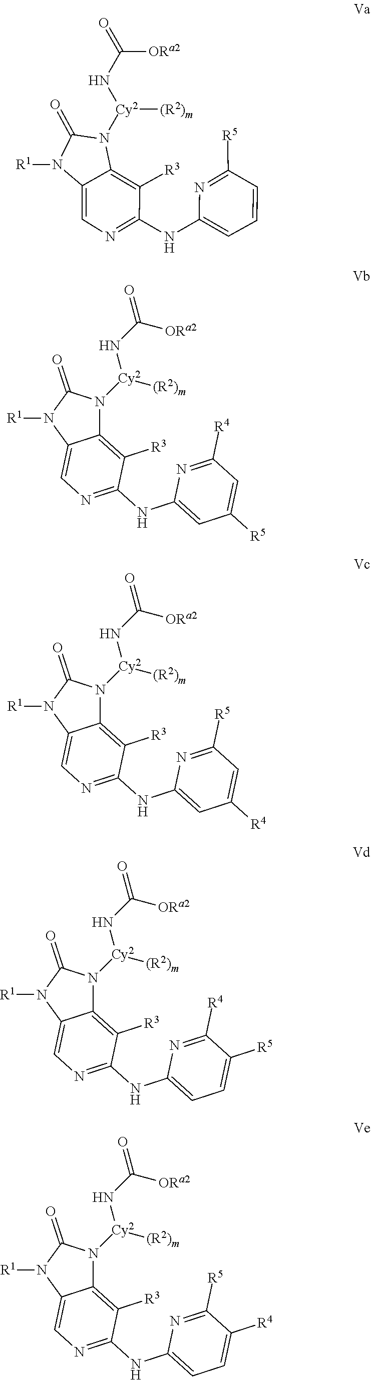

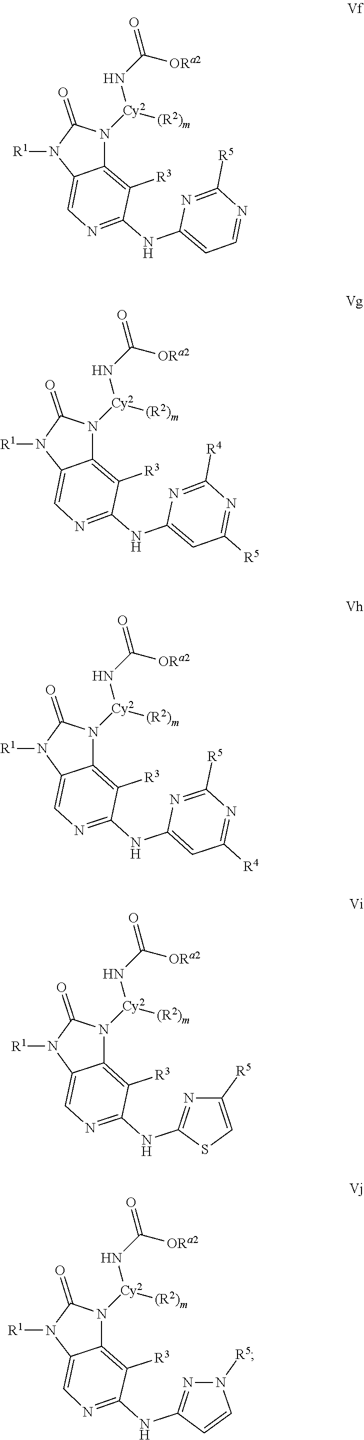

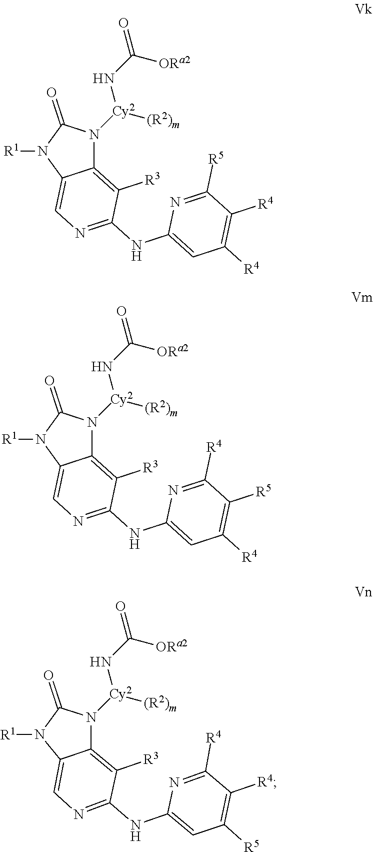

or a pharmaceutically acceptable salt thereof.

In some embodiments, the compound of Formula I is a compound of Formula Va, or a pharmaceutically acceptable salt thereof.

In some embodiments, the compound of Formula I is a compound of Formula Vb, or a pharmaceutically acceptable salt thereof.

In some embodiments, the compound of Formula I is a compound of Formula Vc, or a pharmaceutically acceptable salt thereof.

In some embodiments, the compound of Formula I is a compound of Formula Vd, or a pharmaceutically acceptable salt thereof.

In some embodiments, the compound of Formula I is a compound of Formula Ve, or a pharmaceutically acceptable salt thereof.

In some embodiments, the compound of Formula I is a compound of Formula Vf, or a pharmaceutically acceptable salt thereof.

In some embodiments, the compound of Formula I is a compound of Formula Vg, or a pharmaceutically acceptable salt thereof.

In some embodiments, the compound of Formula I is a compound of Formula Vh, or a pharmaceutically acceptable salt thereof.

In some embodiments, the compound of Formula I is a compound of Formula Vi, or a pharmaceutically acceptable salt thereof.

In some embodiments, the compound of Formula I is a compound of Formula Vj, or a pharmaceutically acceptable salt thereof.

In some embodiments, the compound of Formula I is a compound of Formula Vk, or a pharmaceutically acceptable salt thereof.

In some embodiments, the compound of Formula I is a compound of Formula Vm, or a pharmaceutically acceptable salt thereof.

In some embodiments, the compound of Formula I is a compound of Formula Vn, or a pharmaceutically acceptable salt thereof.

In some embodiments of Formulas Va-Vn, m is 0, 1, 2, 3, or 4.

In some embodiments of Formulas Va-Vn, m is 0, 1, 2, or 3.

In some embodiments of Formulas Va-Vn, m is 0, 1, or 2.

In some embodiments of Formulas Va-Vn, m is 0 or 1.

In some embodiments, the compound of Formula I is a compound of Formula VIa, VIb, VIc, VId, VIe, VIf, VIg, VIh, VIi, VIj, VIk, VIm, or VIn:

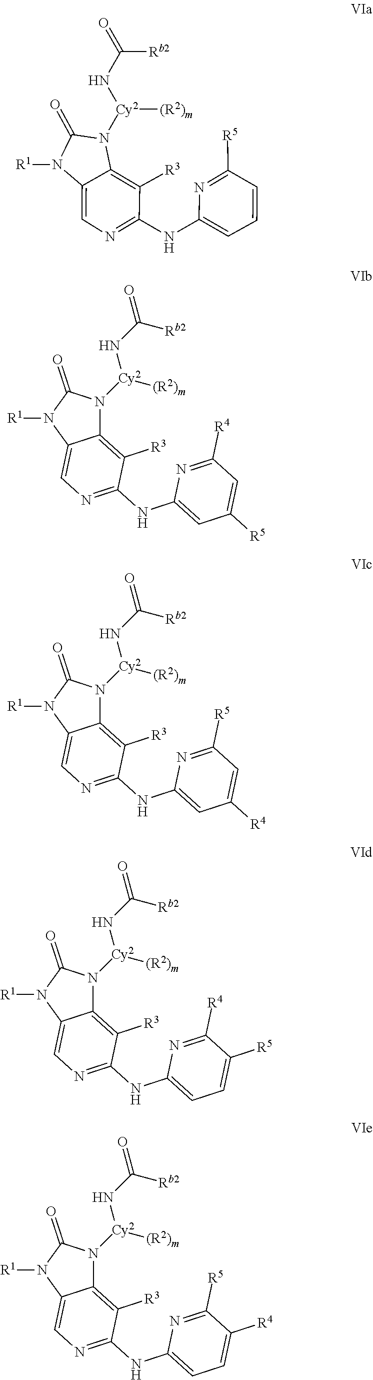

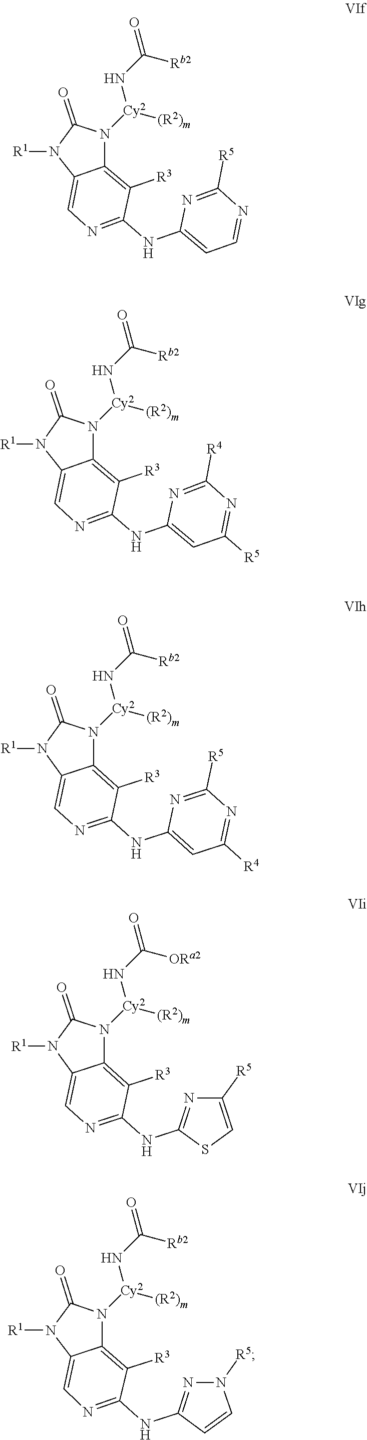

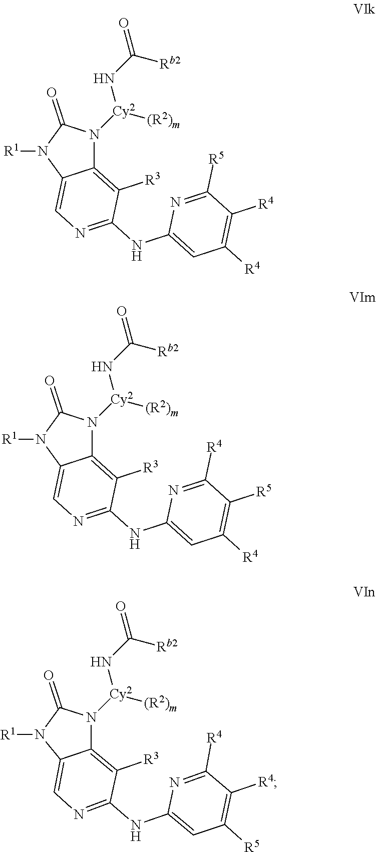

or a pharmaceutically acceptable salt thereof.

In some embodiments, the compound of Formula I is a compound of Formula VIa, or a pharmaceutically acceptable salt thereof.

In some embodiments, the compound of Formula I is a compound of Formula VIb, or a pharmaceutically acceptable salt thereof.

In some embodiments, the compound of Formula I is a compound of Formula VIc, or a pharmaceutically acceptable salt thereof.

In some embodiments, the compound of Formula I is a compound of Formula VId, or a pharmaceutically acceptable salt thereof.

In some embodiments, the compound of Formula I is a compound of Formula VIe, or a pharmaceutically acceptable salt thereof.

In some embodiments, the compound of Formula I is a compound of Formula VIf, or a pharmaceutically acceptable salt thereof.

In some embodiments, the compound of Formula I is a compound of Formula VIg, or a pharmaceutically acceptable salt thereof.

In some embodiments, the compound of Formula I is a compound of Formula VIh, or a pharmaceutically acceptable salt thereof.

In some embodiments, the compound of Formula I is a compound of Formula VIi, or a pharmaceutically acceptable salt thereof.

In some embodiments, the compound of Formula I is a compound of Formula VIj, or a pharmaceutically acceptable salt thereof.

In some embodiments, the compound of Formula I is a compound of Formula VIK, or a pharmaceutically acceptable salt thereof.

In some embodiments, the compound of Formula I is a compound of Formula VIm, or a pharmaceutically acceptable salt thereof.

In some embodiments, the compound of Formula I is a compound of Formula VIn, or a pharmaceutically acceptable salt thereof.

In some embodiments of Formulas VIa-VIn, m is 0, 1, 2, 3, or 4.

In some embodiments of Formulas VIa-VIn, m is 0, 1, 2, or 3.

In some embodiments of Formulas VIa-VIn, m is 0, 1, or 2.

In some embodiments of Formulas VIa-VIn, m is 0 or 1.

In some embodiments, the compound of Formula I is a compound of Formula VIIa, VIIb, VIIc, VIId, VIIe, VIIf, VIIg, VIIh, VIIi, VIIj, VIIk, VIIm, VIIn, or VIIo:

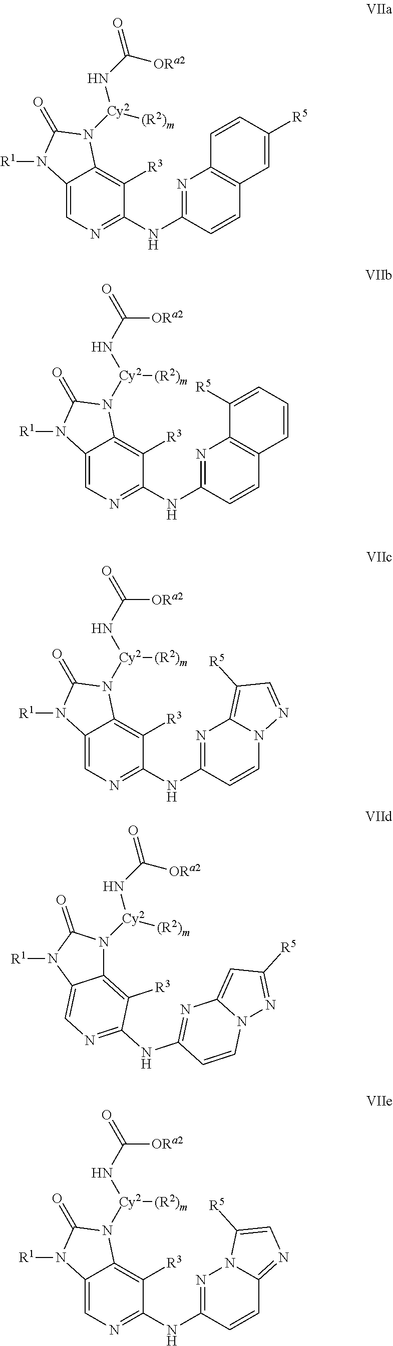

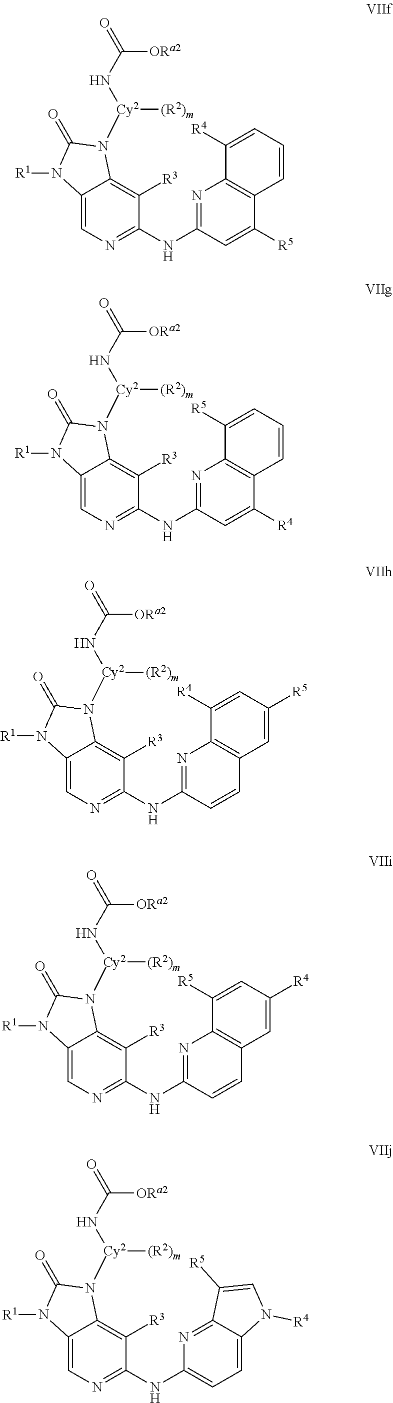

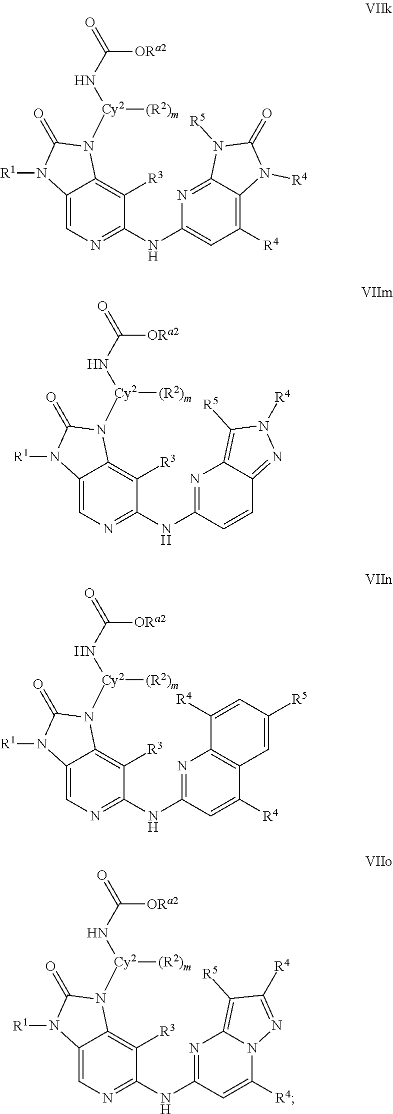

or a pharmaceutically acceptable salt thereof.

In some embodiments, the compound of Formula I is a compound of Formula VIIa. or a pharmaceutically acceptable salt thereof.

In some embodiments, the compound of Formula I is a compound of Formula VIIb, or a pharmaceutically acceptable salt thereof.

In some embodiments, the compound of Formula I is a compound of Formula VIIc, or a pharmaceutically acceptable salt thereof.

In some embodiments, the compound of Formula I is a compound of Formula VIId, or a pharmaceutically acceptable salt thereof.

In some embodiments, the compound of Formula I is a compound of Formula VIIe, or a pharmaceutically acceptable salt thereof.

In some embodiments, the compound of Formula I is a compound of Formula VIIf, or a pharmaceutically acceptable salt thereof.

In some embodiments, the compound of Formula I is a compound of Formula VIIg, or a pharmaceutically acceptable salt thereof.

In some embodiments, the compound of Formula I is a compound of Formula VIIh, or a pharmaceutically acceptable salt thereof.

In some embodiments, the compound of Formula I is a compound of Formula VIIi, or a pharmaceutically acceptable salt thereof.

In some embodiments, the compound of Formula I is a compound of Formula VIIj, or a pharmaceutically acceptable salt thereof.

In some embodiments, the compound of Formula I is a compound of Formula VIIk, or a pharmaceutically acceptable salt thereof.

In some embodiments, the compound of Formula I is a compound of Formula VIIm, or a pharmaceutically acceptable salt thereof.

In some embodiments, the compound of Formula I is a compound of Formula VIIn, or a pharmaceutically acceptable salt thereof.

In some embodiments, the compound of Formula I is a compound of Formula VIIo, or a pharmaceutically acceptable salt thereof.

In some embodiments of Formulas VIIa-VIIo, m is 0, 1, 2, 3, or 4.

In some embodiments of Formulas VIIa-VIIo, m is 0, 1, 2, or 3.

In some embodiments of Formulas VIIa-VIIo, m is 0, 1, or 2.

In some embodiments of Formulas VIIa-VIIo, m is 0 or 1.

In some embodiments, the compound of Formula I is a compound of Formula VIIIa, VIIIb, VIIIc, VIIId, VIIIe, VIIIf, VIIIg, VIIIh, VIIIi, VIIIj, VIIIk, VIIIm, VIIIn, or VIIIo:

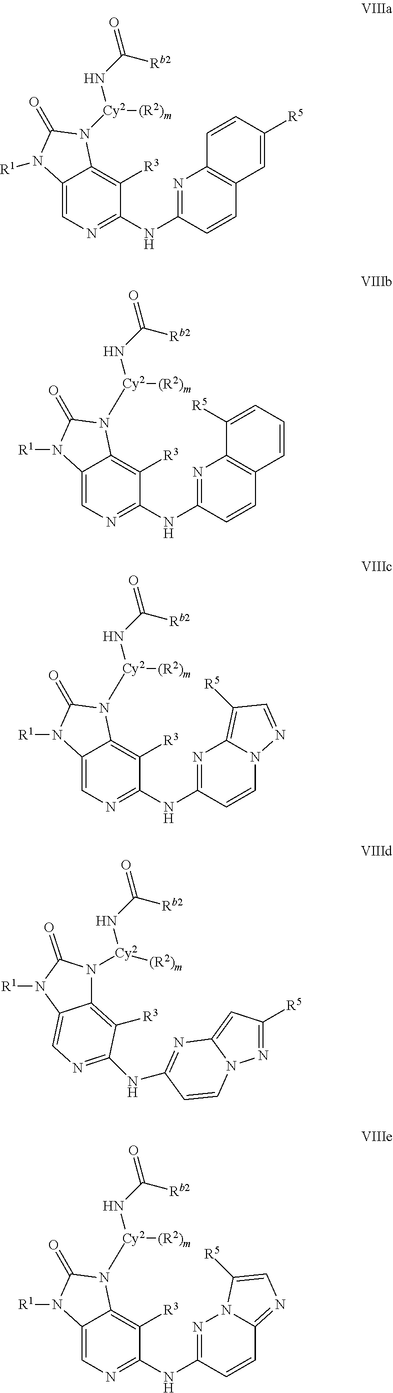

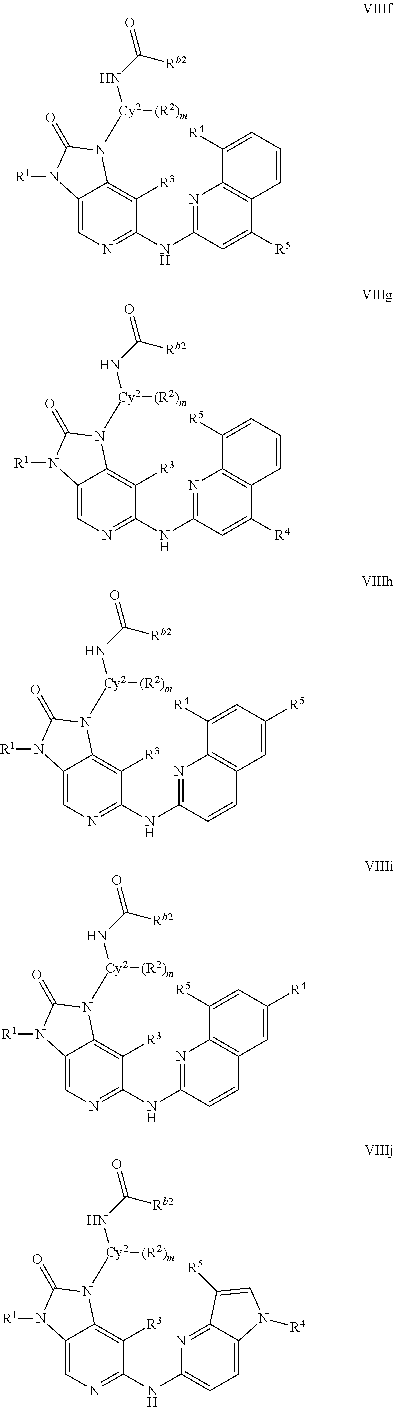

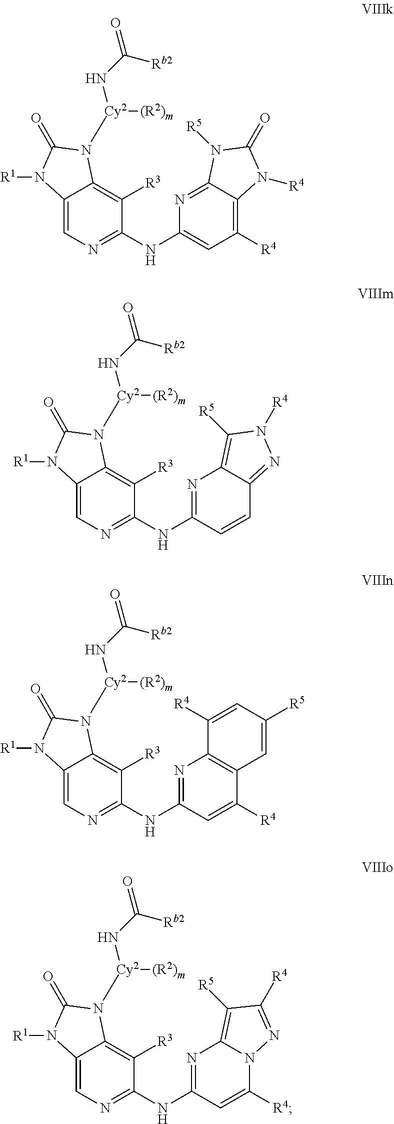

or a pharmaceutically acceptable salt thereof.

In some embodiments, the compound of Formula I is a compound of Formula VIIIa, or a pharmaceutically acceptable salt thereof.

In some embodiments, the compound of Formula I is a compound of Formula VIIIb, or a pharmaceutically acceptable salt thereof.

In some embodiments, the compound of Formula I is a compound of Formula VIIIc, or a pharmaceutically acceptable salt thereof.

In some embodiments, the compound of Formula I is a compound of Formula VIIId, or a pharmaceutically acceptable salt thereof.

In some embodiments, the compound of Formula I is a compound of Formula VIIIe, or a pharmaceutically acceptable salt thereof.

In some embodiments, the compound of Formula I is a compound of Formula VIIIf, or a pharmaceutically acceptable salt thereof.

In some embodiments, the compound of Formula I is a compound of Formula VIIIg, or a pharmaceutically acceptable salt thereof.

In some embodiments, the compound of Formula I is a compound of Formula VIIIh, or a pharmaceutically acceptable salt thereof.

In some embodiments, the compound of Formula I is a compound of Formula VIIIi, or a pharmaceutically acceptable salt thereof.

In some embodiments, the compound of Formula I is a compound of Formula VIIIj, or a pharmaceutically acceptable salt thereof.

In some embodiments, the compound of Formula I is a compound of Formula VIIIk, or a pharmaceutically acceptable salt thereof.

In some embodiments, the compound of Formula I is a compound of Formula VIIIm, or a pharmaceutically acceptable salt thereof.

In some embodiments, the compound of Formula I is a compound of Formula VIIIn, or a pharmaceutically acceptable salt thereof.

In some embodiments, the compound of Formula I is a compound of Formula VIIIo, or a pharmaceutically acceptable salt thereof.

In some embodiments of Formulas VIIIa-VIIIo, m is 0, 1, 2, 3, or 4.

In some embodiments of Formulas VIIIa-VIIIo, m is 0, 1, 2, or 3.

In some embodiments of Formulas VIIIa-VIIIo, m is 0, 1, or 2.

In some embodiments of Formulas VIIIa-VIIIo, m is 0 or 1.

In some embodiments of any of Formulas I-VIIIo, each R⁴ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, OR^{a4}, and NR^{c4}C(O)OR^{a4}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, of R⁴ are each optionally substituted with 1, 2, 3, or 4 independently selected R^{4A} substituents.

In some embodiments of any of Formulas I-VIIIo, each R⁴ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, (4-10 membered heterocycloalkyl)-C₁₋₆ alkyl-, OR^{a4}, and C(O)R^{b4}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, and (4-10 membered heterocycloalkyl)-C₁₋₆ alkyl- of R⁴ are each optionally substituted with 1, 2, 3, or 4 independently selected R^{4A} substituents.

In some embodiments of any of Formulas I-VIIIo, each R⁴ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, of R⁴ are each optionally substituted with 1, 2, 3, or 4 independently selected R^{4A} substituents.

In some embodiments of any of Formulas I-VIIIo, each R⁴ is independently selected from halo, C₁₋₆ alkyl, C₆₋₁₀ aryl, 5-6 membered heteroaryl, 4-10 membered heterocycloalkyl, (4-10 membered heterocycloalkyl)-C₁₋₆ alkyl-, OR^{a4}, and C(O)R^{b4}, wherein the C₁₋₆ alkyl, C₆-10 aryl, 5-6 membered heteroaryl, 4-10 membered heterocycloalkyl, and (4-10 membered heterocycloalkyl)-C₁₋₆ alkyl- of R⁴ are each optionally substituted with 1, 2, 3, or 4 independently selected R^{4A} substituents.

In some embodiments of any of Formulas I-VIIIo, each R⁴ is independently selected from halo, C₁₋₆ alkyl, C₆₋₁₀ aryl, and 4-10 membered heterocycloalkyl, wherein the C₁₋₆ alkyl, C₆₋₁₀ aryl, and 4-10 membered heterocycloalkyl of R⁴ are each optionally substituted with 1, 2, 3, or 4 independently selected R^{4A} substituents.

In some embodiments of any of Formulas I-VIIIo, each R⁴ is independently selected from halo, C₁₋₆ alkyl, phenyl, 5-6 membered heteroaryl, 8-10 membered heterocycloalkyl, (4-10 membered heterocycloalkyl)-C₁₋₆ alkyl-, OR^{a4}, and C(O)R^{b4}, wherein the C₁₋₆ alkyl, phenyl, 5-6 membered heteroaryl, 8-10 membered heterocycloalkyl, (4-10 membered heterocycloalkyl)-C₁₋₆ alkyl- of R⁴ are each optionally substituted with 1, 2, 3, or 4 independently selected R^{4A} substituents.

In some embodiments of any of Formulas I-VIIIo, each R⁴ is independently selected from halo, C₁₋₆ alkyl, phenyl, and 8-10 membered heterocycloalkyl, wherein the C₁₋₆ alkyl, phenyl, and 8-10 membered heterocycloalkyl of R⁴ are each optionally substituted with 1, 2, 3, or 4 independently selected R^{4A} substituents.

In some embodiments of any of Formulas I-VIIIo, each R⁴ is independently selected from fluoro, methyl, isopropyl, methoxy, morpholinylcarbonyl, phenyl, pyrazolyl, piperidinyl, and 2,3-dihydrobenzo[b][1,4]dioxinyl, wherein the methyl, isopropyl, phenyl, pyrazolyl, piperidinyl, and 2,3-dihydrobenzo[b][1,4]dioxinyl of R⁴ are each optionally substituted with 1, 2, 3, or 4 independently selected R^{4A} substituents.

In some embodiments of any of Formulas I-VIIIo, each R⁴ is independently selected from fluoro, methyl, phenyl, and 2,3-dihydrobenzo[b][1,4]dioxinyl, wherein the methyl, phenyl, and 2,3-dihydrobenzo[b][1,4]dioxinyl of R⁴ are each optionally substituted with 1, 2, 3, or 4 independently selected R^{4A} substituents.

In some embodiments of any of Formulas I-VIIIo, R⁴ is selected from C₆₋₁₀ aryl, C₃₋₁₀ cycloalkyl, 5-10 membered heteroaryl, 4-10 membered heterocycloalkyl, OR^{a4}, and NR^{c4}C(O)OR^{a4}, wherein the C₆₋₁₀ aryl, C₃₋₁₀ cycloalkyl, 5-10 membered heteroaryl, and 4-10 membered heterocycloalkyl of R⁴ are each optionally substituted with 1, 2, 3, or 4 independently selected R^{4A} substituents.

In some embodiments of any of Formulas I-VIIIo, R⁴ selected from phenyl, C₃₋₇ cycloalkyl, 5-6 membered heteroaryl, 4-7 membered heterocycloalkyl, OR^{a4}, and NR^{c4}C(O)OR^{a4}, wherein the phenyl, C₃₋₇ cycloalkyl, 5-6 membered heteroaryl, and 4-7 membered heterocycloalkyl of R⁴ are each optionally substituted with 1, 2, 3, or 4 independently selected R^{4A} substituents.

In some embodiments of any of Formulas I-VIIIo, R⁴ selected from phenyl, 5-6 membered heteroaryl, 4-7 membered heterocycloalkyl, OR^{a4}, and NR^{c4}C(O)OR^{a4}, wherein the phenyl, 5-6 membered heteroaryl, and 4-7 membered heterocycloalkyl of R⁴ are each optionally substituted with 1, 2, 3, or 4 independently selected R^{4A} substituents.

In some embodiments of any of Formulas I-VIIIo, R⁴ is selected from phenyl, tetrahydropyranyl, pyrazolyl, OR^{a4}, and NR^{c4}C(O)OR^{a4}, wherein the phenyl, tetrahydropyranyl, and pyrazolyl of R⁴ are each optionally substituted with 1, 2, 3, or 4 independently selected R^{4A} substituents.

In some embodiments of any of Formulas I-VIIIo, each R⁴ is selected from phenyl, tetrahydropyranyl, pyrazolyl, OR^{a4}, and NR^{c4}C(O)OR^{a4}, wherein the phenyl, tetrahydropyranyl, and pyrazolyl of R⁴ are each optionally substituted with 1 or 2 independently selected R^{4A} substituents.

In some embodiments of any of Formulas I-VIIIo, each R⁴ is independently selected from halo, C₁₋₆ alkyl, C₁₋₆ haloalkyl, C₂₋₆ alkenyl, C₂₋₆ alkynyl, and 5-6 membered heteroaryl, wherein each C₁₋₆ alkyl, C₂₋₆ alkenyl, C₂₋₆ alkynyl, and 5-6 membered heteroaryl of R⁴ is optionally substituted with 1, 2, 3, or 4 independently selected R^{4A} substituents.

In some embodiments, R⁴ is independently selected from halo, C₁₋₆ alkyl, C₁₋₆ haloalkyl, and 5-6 membered heteroaryl, wherein each C₁₋₆ alkyl and 5-6 membered heteroaryl of R⁴ is optionally substituted with 1, 2, 3, or 4 independently selected R^{4A} substituents.

In some embodiments of any of Formulas I-VIIIo, each R⁴ is independently selected from halo, C₁₋₆ alkyl, C₁₋₆ haloalkyl, C₂₋₆ alkenyl, and C₂₋₆ alkynyl.

In some embodiments of any of Formulas I-VIIIo, each R⁴ is independently selected from halo, C₁₋₆ alkyl, and C₁₋₆ haloalkyl.

In some embodiments of any of Formulas I-VIIIo, each R⁴ is independently selected from C₁₋₆ haloalkyl.

In some embodiments of any of Formulas I-VIIIo, each R⁴ is independently selected from fluoro, methyl, trifluoromethyl, phenyl, pyrazolyl, tetrahydropyranyl, and 2,3-dihydrobenzo[b][1,4]dioxinyl, wherein the methyl, phenyl, pyrazolyl, tetrahydropyranyl, and 2,3-dihydrobenzo[b][1,4]dioxinyl of R⁴ are each optionally substituted with 1, 2, 3, or 4 independently selected R^{4A} substituents.

In some embodiments of any of Formulas I-VIIIo, each R⁴ is independently selected from fluoro, methyl, hydroxymethyl, hydroxyisopropyl, methoxy, (methylcarbonyl)piperidinyl, methylpyrazolyl, trifluoromethyl, cyanophenyl, pyrazolyl, tetrahydropyranyl, 2,3-dihydrobenzo[b][1,4]dioxinyl, (difluoromethylazetidinyl)methyl, and morpholinylcarbonyl.

In some embodiments of any of Formulas I-VIIIo, each R⁴ is independently selected from fluoro, methyl, trifluoromethyl, cyanophenyl, pyrazolyl, tetrahydropyranyl, and 2,3-dihydrobenzo[b][1,4]dioxinyl.

In some embodiments of any of Formulas I-VIIIo, R⁴ is independently selected from fluoro, methyl, hydroxymethyl, hydroxyisopropyl, methoxy, (methylcarbonyl)piperidinyl, methylpyrazolyl, cyanophenyl, 2,3-dihydrobenzo[b][1,4]dioxinyl, and (difluoromethylazetidinyl)methyl, and morpholinylcarbonyl.

In some embodiments of any of Formulas I-VIIIo, R⁴ is independently selected from fluoro, methyl, cyanophenyl, and 2,3-dihydrobenzo[b][1,4]dioxinyl.

In some embodiments of any of Formulas I-VIIIo, R⁴ is trifluoromethyl.

In some embodiments of any of Formulas I-VIIIo, each R^{a4}, R^{b4}, R^{c4}, and R^{d4} is independently selected from H, C₁₋₆ alkyl, C₁₋₆ haloalkyl, C₂₋₆ alkenyl, and C₂₋₆ alkynyl. In some embodiments of any of Formulas I-VIIIo, each R^{a4} and R^{c4} is independently

selected from H and C₁₋₆ alkyl.

In some embodiments of any of Formulas I-VIIIo, each R^{4A} is independently selected from halo, C₁₋₆ alkyl, C₁₋₆ haloalkyl, C₂₋₆ alkenyl, C₂₋₆ alkynyl, CN, OR^{a41}, and C(O)R^{b41}.

In some embodiments of any of Formulas I-VIIIo, each R^{4A} is independently selected from halo, C₁₋₆ alkyl, C₁₋₆ haloalkyl, C₂₋₆ alkenyl, C₂₋₆ alkynyl, CN, and OR^{a41}.

In some embodiments of any of Formulas I-VIIIo, each R^{a41} and R^{b41} is independently selected from H, C₁₋₆ alkyl, C₁₋₆ haloalkyl, C₂₋₆ alkenyl, and C₂₋₆ alkynyl.

In some embodiments of any of Formulas I-VIIIo, each R^{a41} is independently selected from H, C₁₋₆ alkyl, C₁₋₆ haloalkyl, C₂₋₆ alkenyl, and C₂₋₆ alkynyl.

In some embodiments of any of Formulas I-VIIIo, each R^{a41} is independently selected from H, C₁₋₆ alkyl, and C₁₋₆ haloalkyl.

In some embodiments of any of Formulas I-VIIIo, each R^{a41} is independently selected from H and C₁₋₆ alkyl.

In some embodiments of any of Formulas I-VIIIo, each R^{b41} is independently selected from H, C₁₋₆ alkyl, C₁₋₆ haloalkyl, C₂₋₆ alkenyl, and C₂₋₆ alkynyl.

In some embodiments of any of Formulas I-VIIIo, each R^{b41} is independently selected from H, C₁₋₆ alkyl, and C₁₋₆ haloalkyl.

In some embodiments of any of Formulas I-VIIIo, each R^{b41} is independently selected from H and C₁₋₆ alkyl.

In some embodiments of any of Formulas I-VIIIo, each R^{4A} is independently selected from halo, C₁₋₆ alkyl, C₁₋₆ haloalkyl, C₂₋₆ alkenyl, C₂₋₆ alkynyl, CN, OR^{a41}, and C(O)R^{b41}; and
each R^{a41} and R^{b41} is independently selected from H, C₁₋₆ alkyl, C₁₋₆ haloalkyl, C₂₋₆ alkenyl, and C₂₋₆ alkynyl.

In some embodiments of any of Formulas I-VIIIo, each R^{4A} is independently selected from halo, C₁₋₆ alkyl, C₁₋₆ haloalkyl, C₂₋₆ alkenyl, C₂₋₆ alkynyl, CN, OR^{a41}, and C(O)R^{b4}1; and
each R^{a41} and R^{b41} is independently selected from H, C₁₋₆ alkyl, and C₁₋₆ haloalkyl.

In some embodiments of any of Formulas I-VIIIo, each R^{4A} is independently selected from halo, C₁₋₆ alkyl, C₁₋₆ haloalkyl, C₂₋₆ alkenyl, C₂₋₆ alkynyl, CN, OR^{a41}, and C(O)R^{b41}; and
each R^{a41} and R^{b41} is independently selected from H and C₁₋₆ alkyl.

In some embodiments of any of Formulas I-VIIIo, each R^{4A} is independently selected from halo, C₁₋₆ alkyl, C₁₋₆ haloalkyl, C₂₋₆ alkenyl, C₂₋₆ alkynyl, CN, and OR^{a4}1; and
each R^{a41} is independently selected from H, C₁₋₆ alkyl, C₁₋₆ haloalkyl, C₂₋₆ alkenyl, and C₂₋₆ alkynyl.

In some embodiments of any of Formulas I-VIIIo, each R^{4A} is independently selected from halo, C₁₋₆ alkyl, C₁₋₆ haloalkyl, C₂₋₆ alkenyl, C₂₋₆ alkynyl, CN, and OR^{a41}; and
each R^{a41} is independently selected from H, C₁₋₆ alkyl, and C₁₋₆ haloalkyl.

In some embodiments of any of Formulas I-VIIIo, each R^{4A} is independently selected from halo, C₁₋₆ alkyl, C₁₋₆ haloalkyl, C₂₋₆ alkenyl, C₂₋₆ alkynyl, CN, and OR^{a41}; and
each R^{a41} is independently selected from H and C₁₋₆ alkyl.

In some embodiments of any of Formulas I-VIIIo, each R^{4A} is independently selected from halo, oxo, C₁₋₆ alkyl, C₁₋₆ haloalkyl, C₂₋₆ alkenyl, C₂₋₆ alkynyl, and CN.

In some embodiments, each R^{4A} is independently selected from C₁₋₆ haloalkyl, CN, and OR^{a41}.

In some embodiments, each R^{4A} is independently selected from C₁₋₆ haloalkyl, CN, and OR^{a41}; and
each R^{a41} is independently selected from H and C₁₋₆ alkyl.

In some embodiments of any of Formulas I-VIIIo, each R^{4A} is independently selected from C₁₋₆ alkyl, C₁₋₆ haloalkyl, C₂₋₆ alkenyl, C₂₋₆ alkynyl, and CN.

In some embodiments of any of Formulas I-VIIIo, each R^{4A} is independently selected from C₁₋₆ alkyl, C₁₋₆ haloalkyl, and CN.

In some embodiments of any of Formulas I-VIIIo, each R^{4A} is independently selected from C₁₋₆ alkyl and CN.

In some embodiments of any of Formulas I-VIIIo, each R^{4A} is independently selected from methyl, difluoromethyl, CN, OH, and methylcarbonyl.

In some embodiments of any of Formulas I-VIIIo, each R^{4A} is independently selected from methyl and CN.

In some embodiments of any of Formulas I-VIIIo, each R^{4A} is CN.

In some embodiments, R⁴ is independently selected from C₁₋₆ alkyl, C₁₋₆ haloalkyl, and 5-6 membered heteroaryl, wherein each C₁₋₆ alkyl and 5-6 membered heteroaryl of R⁴ is optionally substituted with 1, 2, 3, or 4 R^{4A} substituents;
each R^{4A} is independently selected from C₁₋₆ haloalkyl, CN, and OR^{a41}; and
each R^{a41} is independently selected from H and C₁₋₆ alkyl.

In some embodiments of any of Formulas I-VIIIo, R⁴ is selected from methyl, ethyl, isopropyl, trifluoromethyl, and pyridinyl, wherein each methyl, ethyl, isopropyl, and pyridinyl of R⁴ is optionally substituted with 1 or 2 R^{4A} substituents independently selected from C₁₋₃ haloalkyl, CN, OH, and C₁₋₃ alkoxy.

In some embodiments of any of Formulas I-VIIIo, R⁵ is selected from C₁₋₆ alkyl, C₃₋₁₂ cycloalkyl, phenyl, 5-12 membered heteroaryl, pyrrolidinyl, piperidinyl, tetrahydropyranyl, 2,3-dihydrobenzo[b][1,4]dioxinyl, 3,4-dihydro-2H-benzo[b][1,4]oxazinyl, azaspiro[2.4]heptanyl, OR^{w2}, NR^{w2}R^{w3}, C(O)R^{w2}, C(O)NR^{w2}R^{w3}, C(O)R^{w2}, NR^{w3}C(O)R^{w2}, NR^{w3}C(O)OR^{w2}, and NR^{w3}S(O)₂R^{w2}, wherein the C₃₋₁₂ cycloalkyl, 5-12 membered heteroaryl, pyrrolidinyl, piperidinyl, 2,3-dihydrobenzo[b][1,4]dioxinyl, 3,4-dihydro-2H-benzo[b][1,4]oxazinyl, and azaspiro[2.4]heptanyl of R⁵ are each optionally substituted with 1, 2, 3, or 4 independently selected R^{5A} substituents; and
wherein the C₁₋₆ alkyl and phenyl of R⁵ are each substituted with 1 R^{w1} substituent and optionally substituted 1 or 2 independently selected R^{5A} substituents; or
the phenyl of R⁵ is substituted with 1 R^{w5} substituent and optionally substituted 1 or 2 independently selected R^{5A} substituents.

In some embodiments of any of Formulas I-VIIIo, R⁵ is selected from C₁₋₆ alkyl, C₃₋₁₂ cycloalkyl, phenyl, triazolyl, pyrazolyl, imidazolyl, pyrrolidinyl, piperidinyl, tetrahydropyranyl, 2,3-dihydrobenzo[b][1,4]dioxinyl, 3,4-dihydro-2H-benzo[b][1,4]oxazinyl, azaspiro[2.4]heptanyl, OR^{w2}, NR^{w2}R^{w3}, C(O)R^{w2}, C(O)NR^{w2}R^{w3}, C(O)R^{w2}, NR^{w3}C(O)R^{w2}, NR^{w3}C(O)OR^{w2}, and NR^{w3}S(O)₂R^{w2}, the C₃₋₁₂ cycloalkyl, triazolyl, imidazolyl, pyrazolyl, pyrrolidinyl, piperidinyl, tetrahydropyranyl, 2,3-dihydrobenzo[b][1,4]dioxinyl, 3,4-dihydro-2H-benzo[b][1,4]oxazinyl, and azaspiro[2.4]heptanyl of R⁵ are each optionally substituted with 1, 2, 3, or 4 independently selected R^{5A} substituents; and wherein the C₁₋₆ alkyl and phenyl of R⁵ are each substituted with 1 R^{w1} substituent and optionally substituted 1 or 2 independently selected R^{5A} substituents; or the phenyl of R⁵ is substituted with 1 R^{w5} substituent and optionally substituted 1 or 2 independently selected R^{5A} substituents.

In some embodiments of any of Formulas I-VIIIo, R⁵ is selected from C₁₋₆ alkyl, C₃₋₇ cycloalkyl, phenyl, triazolyl, pyrazolyl, imidazolyl, pyrrolidinyl, piperidinyl, tetrahydropyranyl, 2,3-dihydrobenzo[b][1,4]dioxinyl, 3,4-dihydro-2H-benzo[b][1,4]oxazinyl, azaspiro[2.4]heptanyl, OR^{w2}, NHR^{w2}, C(O)R^{w2}, C(O)NHR^{w2}, C(O)R^{w2}, NHC(O)R^{w2}, NHC(O)OR^{w2}, and NHS(O); R^{w2}, wherein the C₃₋₁₂ cycloalkyl, triazolyl, imidazolyl, pyrazolyl, pyrrolidinyl, piperidinyl, tetrahydropyranyl, 2,3-dihydrobenzo[b][1,4]dioxinyl, 3,4-dihydro-2H-benzo[b][1,4]oxazinyl, and azaspiro[2.4]heptanyl of R⁵ are each optionally substituted with 1, 2, 3, or 4 independently selected R^{5A} substituents; and
wherein the C₁₋₆ alkyl and phenyl of R⁵ are each substituted with 1 R^{w1} substituent and optionally substituted 1 or 2 independently selected R^{5A} substituents; or
the phenyl of R⁵ is substituted with 1 R^{w5} substituent and optionally substituted 1 or 2 independently selected R^{5A} substituents.

In some embodiments of any of Formulas I-VIIIo, R⁵ is selected from C₃₋₁₂ cycloalkyl, 5-12 membered heteroaryl, NR^{w2}R^{w3}, and C(O)R^{w2}, wherein the C₃₋₁₂ cycloalkyl and 5-12 membered heteroaryl of R⁵ are each optionally substituted with 1, 2, 3, or 4 independently selected R^{5A} substituents.

In some embodiments of any of Formulas I-VIIIo, R⁵ is selected from C₃₋₁₂ cycloalkyl, 5-12 membered heteroaryl, NHR^{w2}, and C(O)R^{w2}, wherein the C₃₋₁₂ cycloalkyl and 5-12 membered heteroaryl of R⁵ are each optionally substituted with 1, 2, 3, or 4 independently selected R^{5A} substituents.

In some embodiments of any of Formulas I-VIIIo, R⁵ is selected from C₁₋₆ alkyl, C₃₋₇ cycloalkyl, phenyl, 5-10 membered heteroaryl, pyrrolidinyl, piperidinyl, 2,3-dihydrobenzo[b][1,4]dioxinyl, 3,4-dihydro-2H-benzo[b][1,4]oxazinyl, azaspiro[2.4]heptanyl, OR^{w2}, NHR^{w2}, C(O)R^{w2}, and C(O)NHR^{w2}.
wherein the C₁₋₆ alkyl and phenyl of R⁵ are each substituted with 1 R¹ substituent and optionally substituted 1 or 2 independently selected R^{5A} substituents; or
the phenyl of R⁵ is substituted with 1 R^{w5} substituent and optionally substituted 1 or 2 independently selected R^{5A} substituents; and
the C₃₋₇ cycloalkyl, 5-10 membered heteroaryl, pyrrolidinyl, piperidinyl, 2,3-dihydrobenzo[b][1,4]dioxinyl, 3,4-dihydro-2H-benzo[b][1,4]oxazinyl, and azaspiro[2.4]heptanyl of R⁵ are each optionally substituted with 1, 2, 3, or 4 independently selected R^{5A} substituents.

In some embodiments of any of Formulas I-VIIIo, R⁵ is selected from C₁₋₆ alkyl, C₃₋₁₂ cycloalkyl, C₆₋₁₀ aryl, 5-12 membered heteroaryl, pyrrolidinyl, piperidinyl, 2,3-dihydrobenzo[b][1,4]dioxinyl, 3,4-dihydro-2H-benzo[b][1,4]oxazinyl, azaspiro[2.4]heptanyl, OR^{w2}, NR^{w2}R^{w3}, C(O)R^{w2}, and C(O)NR^{w2}R^{w3};
wherein the C₁₋₆ alkyl and C₆₋₁₀ aryl of R⁵ are each substituted with 1 R^{w1} substituent and optionally substituted 1 or 2 independently selected R^{5A} substituents; or
the C₆₋₁₀ aryl of R⁵ is substituted with 1 R^{w5} substituent and optionally substituted 1 or 2 independently selected R^{5A} substituents; and
the C₃₋₁₂ cycloalkyl, 5-12 membered heteroaryl, pyrrolidinyl, piperidinyl, 2,3-dihydrobenzo[b][1,4]dioxinyl, 3,4-dihydro-2H-benzo[b][1,4]oxazinyl, and azaspiro[2.4]heptanyl of R⁵ are each optionally substituted with 1, 2, 3, or 4 independently selected R^{5A} substituents.

In some embodiments of any of Formulas I-VIIIo, R⁵ is selected from C₁₋₆ alkyl, C₃₋₁₂ cycloalkyl, C₆₋₁₀ aryl, 5-12 membered heteroaryl, pyrrolidinyl, piperidinyl, 2,3-dihydrobenzo[b][1,4]dioxinyl, 3,4-dihydro-2H-benzo[b][1,4]oxazinyl, azaspiro[2.4]heptanyl, OR^{w2}, NHR^{w2}, C(O)R^{w2}, and C(O)NHR^{w2};
wherein the C₁₋₆ alkyl and C₆₋₁₀ aryl of R⁵ are each substituted with 1 R^{w1} substituent and optionally substituted 1 or 2 independently selected R^{5A} substituents; or
the C₆₋₁₀ aryl of R⁵ is substituted with 1 R^{w5} substituent and optionally substituted 1 or 2 independently selected R^{5A} substituents; and
the C₃₋₁₂ cycloalkyl, 5-12 membered heteroaryl, pyrrolidinyl, piperidinyl, 2,3-dihydrobenzo[b][1,4]dioxinyl, 3,4-dihydro-2H-benzo[b][1,4]oxazinyl, and azaspiro[2.4]heptanyl of R⁵ are each optionally substituted with 1, 2, 3, or 4 independently selected R^{5A} substituents.

In some embodiments of any of Formulas I-VIIIo, R⁵ is selected from —CH₂R^{w1}, C₃₋₇ cycloalkyl, -phenyl-R^{w1}, -phenyl-R^{w5}, pyrazolyl, pyridinyl, indazolyl, quinolinyl, pyrrolidinyl, piperidinyl, 2,3-dihydrobenzo[b][1,4]dioxinyl, 3,4-dihydro-2H-benzo[b][1,4]oxazinyl, azaspiro[2.4]heptanyl, OR^{w2}, NHR^{w2}, C(O)R^{w2}, and C(O)NHR^{w2}, wherein the C₃₋₇ cycloalkyl, phenyl, pyrazolyl, pyridinyl, indazolyl, quinolinyl, pyrrolidinyl, piperidinyl, 2,3-dihydrobenzo[b][1,4]dioxinyl, 3,4-dihydro-2H-benzo[b][1,4]oxazinyl, and azaspiro[2.4]heptanyl of R⁵ are each optionally substituted with 1, 2, 3, or 4 independently selected R^{5A} substituents.

In some embodiments of any of Formulas I-VIIIo, R⁵ is selected from triazolyl, pyrazolyl, piperidinyl, tetrahydropyranyl, NR^{w2}R^{w3}, NR^{w3}C(O)R^{w2}, NR^{w3}C(O)OR^{w2}, and NR^{w3}S(O)₂R^{w2}, wherein the triazolyl, pyrazolyl, piperidinyl, and tetrahydropyranyl of R⁵ are each optionally substituted with 1, 2, 3, or 4 independently selected R^{5A} substituents.

In some embodiments of any of Formulas I-VIIIo, R⁵ is selected from triazolyl, pyrazolyl, piperidinyl, tetrahydropyranyl, NHR^{w2}, NHC(O)R^{w2}, NHC(O)OR^{w2}, and NHS(O)¿R^{w2}, wherein the triazolyl, pyrazolyl, piperidinyl, and tetrahydropyranyl of R⁵ are each optionally substituted with 1, 2, 3, or 4 independently selected R^{5A} substituents.

In some embodiments of any of Formulas I-VIIIo, R⁵ is selected from C₃₋₇ cycloalkyl, 5-6 membered heteroaryl, NHR^{w2}, and C(O)R^{w2}, wherein the C₃₋₇ cycloalkyl and 5-6 membered heteroaryl of R⁵ are each optionally substituted with 1, 2, 3, or 4 independently selected R^{5A} substituents.

In some embodiments of any of Formulas I-VIIIo, R⁵ is selected from cyclopropyl, pyrazolyl, imidazolyl, NHR^{w2}, and C(O)R^{w2}, wherein the cyclopropyl, pyrazolyl, imidazolyl, of R⁵ are each optionally substituted with 1, 2, 3, or 4 independently selected R^{5A} substituents.

In some embodiments of any of Formulas I-VIIIo. R^{w1} is selected from C₃₋₁₂ cycloalkyl, 4-12 membered heterocycloalkyl, OR^{w4}, and NHR^{w4}, wherein the C₃₋₁₂ cycloalkyl and 4-12 membered heterocycloalkyl or R^{w1} are each optionally substituted by 1, 2, 3, or 4 independently selected R^{5A} substituents.

In some embodiments of any of Formulas I-VIIIo, R^{w1} is selected from C₃₋₇ cycloalkyl, 4-7 membered heterocycloalkyl, OR^{w4}, and NHR^{w4}, wherein the C₃₋₇ cycloalkyl and 4-7 membered heterocycloalkyl or R^{w1} are each optionally substituted by 1, 2, 3, or 4 independently selected R^{5A} substituents.

In some embodiments of any of Formulas I-VIIIo, R^{w1} is selected from cyclopropyl, fluoropyrrolidinyl, morpholinyl, trifluoromethoxy, and cyclopentylamino.

In some embodiments of any of Formulas I-VIIIo, R^{w2} is selected from C₃₋₇ cycloalkyl, bicyclic 8-12 membered heteroaryl, monocyclic 4-7 membered heterocycloalkyl, and bicyclic 8-12 membered heterocycloalkyl, wherein the C₃₋₇ cycloalkyl, bicyclic 8-12 membered heteroaryl, monocyclic 4-7 membered heterocycloalkyl, and bicyclic 8-12 membered heterocycloalkyl of R^{w2} are each optionally substituted with 1, 2, 3, or 4 independently selected R^{5A} substituents.

In some embodiments of any of Formulas I-VIIIo, R^{w2} is selected from C₃₋₇ cycloalkyl, 4-8 membered monocyclic heterocycloalkyl, and 6-10 membered bicyclic heterocycloalkyl, wherein the C₃₋₇ cycloalkyl, 4-8 membered monocyclic heterocycloalkyl, and 6-10 membered bicyclic heterocycloalkyl of R^{w2} are each optionally substituted with 1, 2, 3, or 4 independently selected R^{5A} substituents.

In some embodiments of any of Formulas I-VIIIo, R^{w2} is independently selected from C₃₋₁₂ cycloalkyl and 4-12 membered heterocycloalkyl, wherein the C₃₋₁₂ cycloalkyl and 4-12 membered heterocycloalkyl of R^{w2} are each optionally substituted with 1, 2, 3, or 4 independently selected R^{5A} substituents;

In some embodiments of any of Formulas I-VIIIo, each R^{w2} is independently selected from C₃₋₁₂ cycloalkyl, 5-12 membered heteroaryl, and 4-12 membered heterocycloalkyl, wherein the C₃₋₁₂ cycloalkyl, 5-12 membered heteroaryl, and 4-12 membered heterocycloalkyl of R^{w2} are each optionally substituted with 1, 2, 3, or 4 independently selected R^{5A} substituents;

In some embodiments of any of Formulas I-VIIIo, each R^{w2} is independently selected from C₃₋₁₂ cycloalkyl, 5-12 membered heteroaryl, and 4-12 membered heterocycloalkyl, wherein the C₃₋₁₂ cycloalkyl, 5-12 membered heteroaryl, and 4-12 membered heterocycloalkyl of R^{w2} are each optionally substituted with 1, 2, 3, or 4 independently selected R^{5A} substituents;

In some embodiments of any of Formulas I-VIIIo, each R^{w2} is independently selected from C₃₋₇ cycloalkyl, 5-6 membered heteroaryl, and 4-7 membered heterocycloalkyl, wherein the C₃₋₇ cycloalkyl, 5-6 membered heteroaryl, and 4-7 membered heterocycloalkyl of R^{w2} are each optionally substituted with 1, 2, 3, or 4 independently selected R^{5A} substituents.

In some embodiments of any of Formulas I-VIIIo, R^{w2} is selected from cyclopropyl, cyclobutyl, cyclopentyl, cyclohexyl, pyrrolidinyl, piperidinyl, morpholinyl, tetrahydropyranyl, oxazolyl, octahydro-2H-pyrido[1,2-a]pyrazinyl, quinolinyl, 1,6-diazaspiro[3.3]heptanyl, 3-azabicyclo[3.1.0]hexanyl, 2-oxa-5-azabicyclo[2.2.1]heptanyl, and 2-oxa-5-azabicyclo[4.1.0]heptanyl, wherein the cyclopropyl, cyclobutyl, cyclopentyl, cyclohexyl, pyrrolidinyl, piperidinyl, morpholinyl, tetrahydropyranyl, oxazolyl, octahydro-2H-pyrido[1,2-a]pyrazinyl, quinolinyl, 1,6-diazaspiro[3.3]heptanyl, 3-azabicyclo[3.1.0]hexanyl, 2-oxa-5-azabicyclo[2.2.1]heptanyl, and 2-oxa-5-azabicyclo[4.1.0]heptanyl of R^{w2} are each optionally substituted with 1, 2, 3, or 4 independently selected R^{5A} substituents.

In some embodiments of any of Formulas I-VIIIo, R^{w2} is selected from cyclobutyl, cyclopentyl, cyclohexyl, pyrrolidinyl, piperidinyl, morpholinyl, tetrahydropyranyl, octahydro-2H-pyrido[1,2-a]pyrazinyl, quinolinyl, 1,6-diazaspiro[3.3]heptanyl, 3-azabicyclo[3.1.0]hexanyl, 2-oxa-5-azabicyclo[2.2.1]heptanyl, and 2-oxa-5-azabicyclo[4.1.0]heptanyl, wherein the cyclobutyl, cyclopentyl, cyclohexyl, pyrrolidinyl, piperidinyl, morpholinyl, tetrahydropyranyl, octahydro-2H-pyrido[1,2-a]pyrazinyl, quinolinyl, 1,6-diazaspiro[3.3]heptanyl, 3-azabicyclo[3.1.0]hexanyl, 2-oxa-5-azabicyclo[2.2.1]heptanyl, and 2-oxa-5-azabicyclo[4.1.0]heptanyl of R^{w2} are each optionally substituted with 1, 2, 3, or 4 independently selected R^{5A} substituents.

In some embodiments of any of Formulas I-VIIIo, R^{w2} is selected from cyclopentyl, cyclohexyl, pyrrolidinyl, piperidinyl, morpholinyl, tetrahydropyranyl, octahydro-2H-pyrido[1,2-a]pyrazinyl, quinolinyl, 1,6-diazaspiro[3.3]heptanyl, 3-azabicyclo[3.1.0]hexanyl, 2-oxa-5-azabicyclo[2.2.1]heptanyl, and 2-oxa-5-azabicyclo[4.1.0]heptanyl, wherein the cyclopentyl, cyclohexyl, pyrrolidinyl, piperidinyl, morpholinyl, tetrahydropyranyl, octahydro-2H-pyrido[1,2-a]pyrazinyl, quinolinyl, 1,6-diazaspiro[3.3]heptanyl, 3-azabicyclo[3.1.0]hexanyl, 2-oxa-5-azabicyclo[2.2.1]heptanyl, and 2-oxa-5-azabicyclo[4.1.0]heptanyl of R^{w2} are each optionally substituted with 1, 2, 3, or 4 independently selected R^{5A} substituents.

In some embodiments of any of Formulas I-VIIIo, R^{w2} is selected from cyclobutyl, cyclopentyl, piperidinyl, morpholinyl, 1,6-diazaspiro[3.3]heptanyl, 3-azabicyclo[3.1.0]hexanyl, 2-oxa-5-azabicyclo[2.2.1]heptanyl, and 2-oxa-5-azabicyclo[4.1.0]heptanyl, wherein the cyclobutyl, cyclopentyl, piperidinyl, morpholinyl, 1,6-diazaspiro[3.3]heptanyl, 3-azabicyclo[3.1.0]hexanyl, 2-oxa-5-azabicyclo[2.2.1]heptanyl, and 2-oxa-5-azabicyclo[4.1.0]heptanyl of R^{w2} are each optionally substituted with 1, 2, 3, or 4 independently selected R^{5A} substituents.

In some embodiments of any of Formulas I-VIIIo, R^{w2} is selected from cyclopentyl, piperidinyl, morpholinyl, 1,6-diazaspiro[3.3]heptanyl, 3-azabicyclo[3.1.0]hexanyl, 2-oxa-5-azabicyclo[2.2.1]heptanyl, and 2-oxa-5-azabicyclo[4.1.0]heptanyl, wherein the cyclopentyl, piperidinyl, morpholinyl, 1,6-diazaspiro[3.3]heptanyl, 3-azabicyclo[3.1.0]hexanyl, 2-oxa-5-azabicyclo[2.2.1]heptanyl, and 2-oxa-5-azabicyclo[4.1.0]heptanyl of R² are each optionally substituted with 1, 2, 3, or 4 independently selected R^{5A} substituents.

In some embodiments of any of Formulas I-VIIIo, R^{w2} is selected from cyclobutyl, cyclopentyl, cyclohexyl, quinolinyl, tetrahydropyranyl, piperidinyl, morpholinyl, pyrrolidinyl, and octahydro-2H-pyrido[1,2-a]pyrazinyl, wherein the cyclobutyl, cyclopentyl, cyclohexyl, quinolinyl, tetrahydropyranyl, piperidinyl, morpholinyl, pyrrolidinyl, and octahydro-2H-pyrido[1,2-a]pyrazinyl of R^{w2} are each optionally substituted with 1, 2, 3, or 4 independently selected R^{5A} substituents.

In some embodiments of any of Formulas I-VIIIo, R^{w2} is selected from cyclopentyl, cyclohexyl, quinolinyl, tetrahydropyranyl, piperidinyl, morpholinyl, pyrrolidinyl, and octahydro-2H-pyrido[1,2-a]pyrazinyl, wherein the cyclopentyl, cyclohexyl, quinolinyl, tetrahydropyranyl, piperidinyl, morpholinyl, pyrrolidinyl, and octahydro-2H-pyrido[1,2-a]pyrazinyl of R^{w2} are each optionally substituted with 1, 2, 3, or 4 independently selected R^{5A} substituents.

In some embodiments of any of Formulas I-VIIIo, each R^{w2} is independently selected from cyclopropyl, cyclobutyl, oxazolyl, and tetrahydropyranyl, wherein the cyclopropyl, cyclobutyl, oxazolyl, and tetrahydropyranyl of R^{w2} are each optionally substituted with 1, 2, 3, or 4 independently selected R^{5A} substituents.

In some embodiments of any of Formulas I-VIIIo, each R^{w3} is independently selected from H and C₁₋₆ alkyl.

In some embodiments of any of Formulas I-VIIIo, each R^{w3} is H.

In some embodiments of any of Formulas I-VIIIo, each R^{w4} is independently selected from C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl.

In some embodiments of any of Formulas I-VIIIo, each R^{w4} is selected from C₁₋₆ haloalkyl and C₃₋₇ cycloalkyl.

In some embodiments of any of Formulas I-VIIIo, R^{w5} is C₁₋₆ alkyl which is substituted with CN or OR^{a51}.

In some embodiments of any of Formulas I-VIIIo, R^{w5} is selected from cyanomethyl and hydroxymethyl.

In some embodiments of any of Formulas I-VIIIo, each R^{5A} is independently selected from halo, oxo, C₁₋₆ alkyl, C₁₋₆ haloalkyl, C₂₋₆ alkenyl, C₂₋₆ alkynyl, CN, NO₂, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{c51}R^{d51}, NR^{c51}NR^{c51}R^{d51} NR^{e51}C(O)R^{b51}, NR^{c51}C(O)OR^{a51}, NR^{c51}C(O)NR^{c51}R^{d51}, NR^{c51}S(O)R^{b51}, NR^{c51}S(O)NR^{c51}R^{d51}, NR^{c51}S(O)₂R^{b51}, NR^{c51}S(O)₂NR^{c51}R^{d51}, S(O)R^{b51}, S(O)NR^{c51}R^{d51}, S(O)₂R^{b51}, and S(O)₂NR^{c51}R^{d51}, wherein each C₁₋₆ alkyl, C₂₋₆ alkenyl, and C₂₋₆ alkynyl of R^{5A} is optionally substituted with 1, 2, 3, or 4 independently selected R^{5B} substituents.

In some embodiments, each R^{5B} is independently selected from OR^{a52}.

In some embodiments, each R^{a52} is independently selected from H, C₁₋₆ alkyl, and C₁₋₆ haloalkyl.

In some embodiments of any of Formulas I-VIIIo, each R^{5A} is independently selected from halo, oxo, C₁₋₆ alkyl, C₁₋₆ haloalkyl, C₂₋₆ alkenyl, C₂₋₆ alkynyl, CN, NO₂, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{c51}R^{d51}, NR^{c51}NR^{c51}R^{d51} NR^{c51}C(O)R^{b51}, NR^{e51}C(O)OR^{a51}, NR^{c51}C(O)NR^{c51}R^{d51}, NR^{e51}S(O)R^{b51}, NR^{c51}S(O)NR^{c51}R^{d51}, NR^{e51}S(O)₂R^{b51}, NR^{c51}S(O)₂NR^{c51}R^{d51}, S(O)R^{b51}, S(O)NR^{c51}R^{d51}, S(O)₂R^{b51}, and S(O)₂NR^{c51}R^{d51}, wherein each C₁₋₆ alkyl of R^{5A} is optionally substituted with OH.

In some embodiments of any of Formulas I-VIIIo, each R^{5A} is independently selected from halo, oxo, C₁₋₆ alkyl, C₁₋₆ haloalkyl, C₂₋₆ alkenyl, C₂₋₆ alkynyl, CN, NO₂, OR^{a51}, SR^{a51}, NHOR^{a51}, C(O)R^{b51}, C(O)NR^{c51}R^{d51}, C(O)NR^{e51}(OR^{a51}), C(O)OR^{a51}, OC(O)R^{b51}. OC(O)NR^{c51}R^{d51}, NR^{d51}R^{d51}, NR^{c51}NR^{c51}R^{d51} NR^{c51}C(O)R^{b51}, NR^{c51}C(O)OR^{a51}, NR^{c51}C(O)NR^{c51}R^{d51}, NR^{c51}S(O)R^{b51}, NR^{e51}S(O)NR^{c51}R^{d51}, NR^{c51}S(O)₂R^{b51}, NR^{c51}S(O)₂NR^{c51}R^{d51}, S(O)R^{b51}, S(O)NR^{c51}R^{d51}, S(O)₂R^{b51}, and S(O)₂NR^{c51}R^{d51}

In some embodiments of any of Formulas I-VIIIo, each R^{5A} is independently selected from halo, C₁₋₆ alkyl, C₁₋₆ haloalkyl, CN, OR^{a51}, C(O)R^{b51}, and C(O)OR^{a51}, wherein the C₁₋₆ alkyl of R^{5A} is optionally substituted with 1, 2, 3, or 4 independently selected R^{5B} substituents.

In some embodiments of any of Formulas I-VIIIo, each R^{5A} is independently selected from halo, C₁₋₆ alkyl, C₁₋₆ haloalkyl, CN, OR^{a51}, C(O)R^{b51}, and C(O)OR^{a51}, wherein the C₁₋₆ alkyl of R^{5A} is optionally substituted with OH.

In some embodiments of any of Formulas I-VIIIo, each R^{5A} is independently selected from halo, C₁₋₆ alkyl, C₁₋₆ haloalkyl, CN, OR^{a51}, C(O)R^{b51}, and C(O)OR^{a51}.

In some embodiments of any of Formulas I-VIIIo, each R^{5A} is independently selected from halo, oxo, C₁₋₆ alkyl, C₁₋₆ haloalkyl, C₂₋₆ alkenyl, C₂₋₆ alkynyl, CN, OR^{a51}, and NR^{c51}R^{d51} wherein each C₁₋₆ alkyl, C₂₋₆ alkenyl, and C₂₋₆ alkynyl of R^{5A} is optionally substituted with 1, 2, 3, or 4 independently selected R^{5B} substituents.

In some embodiments of any of Formulas I-VIIIo, each R^{5A} is independently selected from halo, oxo, C₁₋₆ alkyl, C₁₋₆ haloalkyl, C₂₋₆ alkenyl, C₂₋₆ alkynyl, CN, OR^{a51}, and NR^{e51}R^{d51} wherein each C₁₋₆ alkyl, C₂₋₆ alkenyl, and C₂₋₆ alkynyl of R^{5A} is optionally substituted with OH.

In some embodiments of any of Formulas I-VIIIo, each R^{5A} is independently selected from halo, oxo, C₁₋₆ alkyl, C₁₋₆ haloalkyl, C₂₋₆ alkenyl, C₂₋₆ alkynyl, CN, OR^{a51}, and NR^{c51}R^{d51}

In some embodiments of any of Formulas I-VIIIo, each R^{5A} is independently selected from halo, C₁₋₆ alkyl, C₁₋₆ haloalkyl, CN, OR^{a51}, and NR^{e51}R^{d51}, wherein each C₁₋₆ alkyl of R^{5A} is optionally substituted with 1, 2, 3, or 4 independently selected R^{5B} substituents.

In some embodiments of any of Formulas I-VIIIo, each R^{5A} is independently selected from halo, C₁₋₆ alkyl, C₁₋₆ haloalkyl, CN, OR^{a51}, and NR^{c51}R^{d51}, wherein each C₁₋₆ alkyl of R^{5A} is optionally substituted with OH.

In some embodiments of any of Formulas I-VIIIo, each R^{5A} is independently selected from halo, C₁₋₆ alkyl, C₁₋₆ haloalkyl, CN, OR^{a51}, and NR^{c51}R^{d51}

In some embodiments of any of Formulas I-VIIIo, each R^{5A} is independently selected from halo, C₁₋₆ alkyl, C₁₋₆ haloalkyl, C₂₋₆ alkenyl, C₂₋₆ alkynyl, CN and OR^{a51}.

In some embodiments of any of Formulas I-VIIIo, each R^{5A} is independently selected from halo, C₁₋₆ alkyl, C₁₋₆ haloalkyl, CN, and OR^{a51}.

In some embodiments of any of Formulas I-VIIIo, each R^{5A} is independently selected from halo, C₁₋₆ alkyl, and C(O)OR^{a51}.

In some embodiments of any of Formulas I-VIIIo, each R^{5A} is independently selected from fluoro, methyl, trideuteromethyl, fluoromethyl, difluoromethyl, trifluoromethyl, CN, hydroxy, methoxy, amino and ethoxycarbonyl.

In some embodiments of any of Formulas I-VIIIo, each R^{5A} is independently selected from fluoro, methyl, trideuteromethyl, fluoromethyl, trifluoromethyl, CN, hydroxy, methoxy, amino and ethoxycarbonyl.

In some embodiments of any of Formulas I-VIIIo, each R^{5A} is independently selected from fluoromethyl, and ethoxycarbonyl.

In some embodiments of any of Formulas I-VIIIo, each R^{5A} is independently selected from chloro, fluoro, methyl, hydroxyisopropyl, difluoromethyl, trifluoromethyl, CN, methoxy, and amino.

In some embodiments of any of Formulas I-VIIIo, each R^{5A} is independently selected from fluoro, methyl, trifluoromethyl, CN, methoxy, and amino.

In some embodiments of any of Formulas I-VIIIo, each R^{5A} is independently selected from fluoro, methyl, trideuteromethyl, difluoromethyl, trifluoromethyl, CN, and hydroxy.

In some embodiments of any of Formulas I-VIIIo, each R^{5A} is independently selected from fluoro, methyl, trideuteromethyl, CN, and hydroxy.

In some embodiments of any of Formulas I-VIIIo, each R^{a51}, R^{b51}, R^{c51}, and R^{d51} is independently selected from H, C₁₋₆ alkyl, C₁₋₆ haloalkyl, C₂₋₆ alkenyl, and C₂₋₆ alkynyl.

In some embodiments of any of Formulas I-VIIIo, each R^{a51} and R^{b51} is independently selected from H, C₁₋₆ alkyl, C₁₋₆ haloalkyl, C₂₋₆ alkenyl, and C₂₋₆ alkynyl.

In some embodiments of any of Formulas I-VIIIo, each R^{a51}, R^{b51}, R^{c51}, and R^{d51} is independently selected from H and C₁₋₆ alkyl.

In some embodiments of any of Formulas I-VIIIo, each R^{a51} and R^{b51} is independently selected from H and C₁₋₆ alkyl.

In some embodiments of any of Formulas I-VIIIo, each R^{a51} is independently selected from H and C₁₋₆ alkyl.

In some embodiments of any of Formulas I-VIIIo, each R^{b51} is independently selected from H and C₁₋₆ alkyl.

In some embodiments of any of Formulas I-VIIIo, each R^{c51} is independently selected from H and C₁₋₆ alkyl.

In some embodiments of any of Formulas I-VIIIo, each R^{d51} is independently selected from H and C₁₋₆ alkyl.

In some embodiments of any of Formulas I-VIIIo:
R⁴ selected from C₆₋₁₀ aryl, C₃₋₁₀ cycloalkyl, 5-10 membered heteroaryl, 4-10 membered heterocycloalkyl, OR^{a4}, and NR^{c4}C(O)OR^{a4}, wherein the C₆₋₁₀ aryl, C₃₋₁₀ cycloalkyl, 5-10 membered heteroaryl, and 4-10 membered heterocycloalkyl of R⁴ are each optionally substituted with 1, 2, 3, or 4 independently selected R^{4A} substituents;
each R^{a4}, R^{c4}, and R^{d4} is independently selected from H, C₁₋₆ alkyl, C₁₋₆ haloalkyl, C₂₋₆ alkenyl, and C₂₋₆ alkynyl;
each R^{4A} is independently selected from C₁₋₆ alkyl, C₁₋₆ haloalkyl, C₂₋₆ alkenyl, C₂₋₆ alkynyl, and CN;
R⁵ is selected from triazolyl, pyrazolyl, piperidinyl, tetrahydropyranyl, NR^{w2}R^{w3}, NR^{w3}C(O)R^{w2}, NR^{w3}C(O)OR^{w2}, and NR^{w3}S(O)₂R^{w2}, wherein the triazolyl, pyrazolyl, piperidinyl, and tetrahydropyranyl of R⁵ are each optionally substituted with 1, 2, 3, or 4 independently selected R^{5A} substituents;
each R^{w2} is independently selected from C₃₋₁₂ cycloalkyl, 5-12 membered heteroaryl, and 4-12 membered heterocycloalkyl, wherein the C₃₋₁₂ cycloalkyl, 5-12 membered heteroaryl, and 4-12 membered heterocycloalkyl of R² are each optionally substituted with 1, 2, 3, or 4 independently selected R^{5A} substituents;
each R^{w3} is independently selected from H, C₁₋₆ alkyl, C₁₋₆ haloalkyl, C₂₋₆ alkenyl, and C₂₋₆ alkynyl;
each R^{5A} is independently selected from halo, oxo, C₁₋₆ alkyl, C₁₋₆ haloalkyl, C₂₋₆ alkenyl, C₂₋₆ alkynyl, CN, NO₂, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{c51}R^{d51}, NR^{e51}NR^{e51}R^{d51} NR^{c51}C(O)R^{b51}, NR^{e51}C(O)OR^{a51}, NR^{c51}C(O)NR^{c51}R^{d51}, NR^{c51}S(O)R^{b51}, NR^{c51}S(O)NR^{c51}R^{d51} NR^{c51}S(O)₂R^{b51}, NR^{c51}S(O)₂NR^{c51}R^{d51}, S(O)R^{b51}, S(O)NR^{c51}R^{d51}, S(O)₂R^{b51}, and S(O)₂NR^{c51}R^{d51}; and each R^{a51}, R^{b51}, R^{e51}, and R^{d51} is independently selected from H, C₁₋₆ alkyl, C₁₋₆ haloalkyl, C₂₋₆ alkenyl, and C₂₋₆ alkynyl.

In some embodiments of any of Formulas I-VIIIo:
each R⁴ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, wherein the C₁₋₆ alkyl, C₂₋₆ alkenyl, C₂₋₆ alkynyl, C₆₋₁₀ aryl, C₃-10 cycloalkyl, 5-10 membered heteroaryl, and 4-10 membered heterocycloalkyl, of R⁴ are each optionally substituted with 1, 2, 3, or 4 independently selected R^{4A} substituents;
each R^{4A} is independently selected from halo, oxo, C₁₋₆ alkyl, C₁₋₆ haloalkyl, C₂₋₆ alkenyl, C₂₋₆ alkynyl, and CN:
R⁵ is selected from C₁₋₆ alkyl, C₃₋₁₂ cycloalkyl, C₆₋₁₀ aryl, 5-12 membered heteroaryl, pyrrolidinyl, piperidinyl, 2,3-dihydrobenzo[b][1,4]dioxinyl, 3,4-dihydro-2H-benzo[b][1,4]oxazinyl, azaspiro[2.4]heptanyl, OR^{w2}, NR^{w2}R^{w3}, C(O)R^{w2}, and C(O)NR^{w2}R^{w3}.
wherein the C₁₋₆ alkyl and C₆₋₁₀ aryl of R⁵ are each substituted with 1 R^{w1} substituent and optionally substituted 1 or 2 independently selected R^{5A} substituents; or
the C₆₋₁₀ aryl of R⁵ is substituted with 1 R^{w5} substituent and optionally substituted 1 or 2 independently selected R^{5A} substituents; and
the C₃₋₁₂ cycloalkyl, 5-12 membered heteroaryl, pyrrolidinyl, piperidinyl, 2,3-dihydrobenzo[b][1,4]dioxinyl, 3,4-dihydro-2H-benzo[b][1,4]oxazinyl, and azaspiro[2.4]heptanyl of R⁵ are each optionally substituted with 1, 2, 3, or 4 independently selected R^{5A} substituents;
R^{w1} is selected from C₃₋₁₂ cycloalkyl, 4-12 membered heterocycloalkyl, OR^{w4}, and NHR^{w4}, wherein the C₃₋₁₂ cycloalkyl and 4-12 membered heterocycloalkyl or R^{w1} are each optionally substituted by 1, 2, 3, or 4 independently selected R^{5A} substituents;
each R^{w2} is independently selected from C₃₋₁₂ cycloalkyl, 5-12 membered heteroaryl, and 4-12 membered heterocycloalkyl, wherein the C₃₋₁₂ cycloalkyl, 5-12 membered heteroaryl, and 4-12 membered heterocycloalkyl of R^{w2} are each optionally substituted with 1, 2, 3, or 4 independently selected R^{5A} substituents;
each R^{w3} is independently selected from H, C₁₋₆ alkyl, C₁₋₆ haloalkyl, C₂₋₆ alkenyl, and C₂₋₆ alkynyl;
each R^{w4} is independently selected from C₁₋₆ haloalkyl, C₂₋₆ alkenyl, C₂₋₆ alkynyl, C₆-10 aryl, C₃₋₁₀ cycloalkyl, 5-10 membered heteroaryl, and 4-10 membered heterocycloalkyl:
R^{w5} is C₁₋₆ alkyl which is substituted with CN or OR^{a51};
each R^{5A} is independently selected from halo, oxo, C₁₋₆ alkyl, C₁₋₆ haloalkyl, C₂₋₆ alkenyl, C₂₋₆ alkynyl, CN, OR^{a51}, and NR^{c51}R^{d51}; and
each R^{a51}, R^{e51}, and R^{d51} is independently selected from H, C₁₋₆ alkyl, C₁₋₆ haloalkyl, C₂₋₆ alkenyl, and C₂₋₆ alkynyl.

In some embodiments of any of Formulas I-VIIIo:
each R⁴ is independently selected from halo, C₁₋₆ alkyl, C₁₋₆ haloalkyl, C₂₋₆ alkenyl, and C₂₋₆ alkynyl;
R⁵ is selected from C₃₋₁₂ cycloalkyl, 5-12 membered heteroaryl, NR^{w2}R^{w3}, and C(O)R^{w2}, wherein the C₃₋₁₂ cycloalkyl and 5-12 membered heteroaryl of R⁵ are each optionally substituted with 1, 2, 3, or 4 independently selected R^{5A} substituents;
each R^{w2} is independently selected from C₃₋₁₂ cycloalkyl and 4-12 membered heterocycloalkyl, wherein the C₃₋₁₂ cycloalkyl and 4-12 membered heterocycloalkyl of R^{w2} are each optionally substituted with 1, 2, 3, or 4 independently selected R^{5A} substituents;
each R^{w3} is independently selected from H, C₁₋₆ alkyl, C₁₋₆ haloalkyl, C₂₋₆ alkenyl, or C₂₋₆ alkynyl;
each R^{5A} is independently selected from halo, C₁₋₆ alkyl, C₁₋₆ haloalkyl, C₂₋₆ alkenyl, C₂₋₆ alkynyl, CN and OR^{a51}; and
each R^{a51} is independently selected from H, C₁₋₆ alkyl, C₁₋₆ haloalkyl, C₂₋₆ alkenyl, and C₂₋₆ alkynyl.

In some embodiments of any of Formulas I-VIIIo, each R⁴ is selected from phenyl, tetrahydropyranyl, pyrazolyl, OR^{a4}, and NR^{c4}C(O)OR^{a4}, wherein the phenyl, tetrahydropyranyl, and pyrazolyl of R⁴ are each optionally substituted with 1 or 2 independently selected R^{4A} substituents; and
each R^{a4} and R^{c4} is independently selected from H and C₁₋₆ alkyl; and
each R^{4A} is independently selected from C₁₋₆ alkyl and CN.

In some embodiments of any of Formulas I-VIIIo, R⁵ is selected from triazolyl, pyrazolyl, piperidinyl, tetrahydropyranyl, NHR^{w2}, NHC(O)R^{w2}, NHC(O)OR^{w2}, and NHS(O)—R^{w2}, wherein the triazolyl, pyrazolyl, piperidinyl, and tetrahydropyranyl of R⁵ are each optionally substituted with 1, 2, 3, or 4 independently selected R^{5A} substituents;
each R^{w2} is independently selected from cyclopropyl, cyclobutyl, oxazolyl, and tetrahydropyranyl, wherein the cyclopropyl, cyclobutyl, oxazolyl, and tetrahydropyranyl of R^{w2} are each optionally substituted with 1, 2, 3, or 4 independently selected R^{5A} substituents;
each R^{5A} is independently selected from halo, C₁₋₆ alkyl, and C(O)OR^{a51}; and
each R^{a51} is independently selected from H and C₁₋₆ alkyl.

In some embodiments of any of Formulas I-VIIIo, R^{w1} is selected from cyclopropyl, fluoropyrrolidinyl, morpholinyl, OR^{w4}, and NHR^{w4}; and
R^{w4} is selected from C₁₋₆ haloalkyl and C₃₋₇ cycloalkyl.

In some embodiments of any of Formulas I-VIIIo, R^{w5} is C₁₋₆ alkyl which is substituted with CN or OR^{a51}; and
R^{a51} is selected from H and C₁₋₃ alkyl.

In some embodiments of any of Formulas I-VIIIo, each R^{5A} is independently selected from halo, oxo, C₁₋₆ alkyl, C₁₋₆ haloalkyl, C₂₋₆ alkenyl, C₂₋₆ alkynyl, CN, NO₂, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{c51}R^{d51}, NR^{c51}NR^{c51}R^{d51}, NR^{c51}C(O)R^{b51}, NR^{c51}C(O)OR^{a51}, NR^{c51}C(O)NR^{c51}R^{d51}, NR^{c51}S(O)R^{b51}, NR^{c51}S(O)NR^{c51}R^{d51}, NR^{c51}S(O)₂R^{b51}, NR^{c51}S(O)₂NR^{c51}R^{d51}, S(O)R^{b51}, S(O)NR^{c51}R^{d51}, S(O)₂R^{b51}, and S(O)—NR^{c51}R^{d51}; and
each R^{a51}, R^{b51}, R^{e51}, and R^{d51} is independently selected from H, C₁₋₆ alkyl, C₁₋₆ haloalkyl, C₂₋₆ alkenyl, and C₂₋₆ alkynyl.

In some embodiments of any of Formulas I-VIIIo, each R^{5A} is independently selected from halo, C₁₋₆ alkyl, C₁₋₆ haloalkyl, CN, OR^{a51}, C(O)R^{b51}, and C(O)OR^{a51}; and
each R^{a51} and R^{b51} is independently selected from H, C₁₋₆ alkyl, C₁₋₆ haloalkyl, C₂₋₆ alkenyl, and C₂₋₆ alkynyl.

In some embodiments of any of Formulas I-VIIIo, each R^{5A} is independently selected from halo, C₁₋₆ alkyl, and C(O)OR^{a51}; and
each R^{a51} is independently selected from H and C₁₋₆ alkyl.

In some embodiments of any of Formulas I-VIIIo, each R^{5A} is independently selected from halo, C₁₋₆ alkyl, C₁₋₆ haloalkyl, CN, OR^{a51}, and NR^{c51}R^{d51}; and
each R^{a51}, R^{c51}, and R^{d51} is independently selected from H and C₁₋₆ alkyl.

In some embodiments of any of Formulas I-VIIIo, each R^{5A} is independently selected from halo, C₁₋₆ alkyl, C₁₋₆ haloalkyl, CN, and OR^{a51}; and
each R^{a51} is independently selected from H and C₁₋₆ alkyl.

In some embodiments, the compound of Formula I is a compound of Formula II:

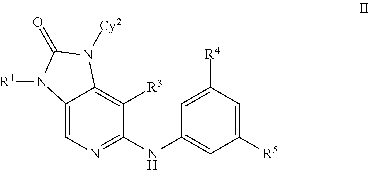

or a pharmaceutically acceptable salt thereof, wherein:
R⁴ selected from C₆₋₁₀ aryl, C₃₋₁₀ cycloalkyl, 5-10 membered heteroaryl, 4-10 membered heterocycloalkyl, OR^{a4}, and NR^{c4}C(O)OR^{a4}, wherein the C₆₋₁₀ aryl, C₃₋₁₀ cycloalkyl, 5-10 membered heteroaryl, and 4-10 membered heterocycloalkyl of R⁴ are each optionally substituted with 1, 2, 3, or 4 independently selected R^{4A} substituents;
each R^{a4}, R^{c4}, and R^{d4} is independently selected from H, C₁₋₆ alkyl, C₁₋₆ haloalkyl, C₂₋₆ alkenyl, and C₂₋₆ alkynyl;
each R^{4A} is independently selected from C₁₋₆ alkyl, C₁₋₆ haloalkyl, C₂₋₆ alkenyl, C₂₋₆ alkynyl, and CN:
R⁵ is selected from triazolyl, pyrazolyl, piperidinyl, tetrahydropyranyl, NR^{w2}R^{w3}, NR^{w3}C(O)R^{w2}, NR^{w3}C(O)OR^{w2}, and NR^{w3}S(O)₂R^{w2}, wherein the triazolyl, pyrazolyl, piperidinyl, and tetrahydropyranyl of R⁵ are each optionally substituted with 1, 2, 3, or 4 independently selected R^{5A} substituents;
each R^{w2} is independently selected from C₃₋₁₂ cycloalkyl, 5-12 membered heteroaryl, and 4-12 membered heterocycloalkyl, wherein the C₃₋₁₂ cycloalkyl, 5-12 membered heteroaryl, and 4-12 membered heterocycloalkyl of R² are each optionally substituted with 1, 2, 3, or 4 independently selected R^{5A} substituents;
each R^{w3} is independently selected from H, C₁₋₆ alkyl, C₁₋₆ haloalkyl, C₂₋₆ alkenyl, and C₂₋₆ alkynyl;
each R^{5A} is independently selected from halo, oxo, C₁₋₆ alkyl, C₁₋₆ haloalkyl, C₂₋₆ alkenyl, C₂₋₆ alkynyl, CN, NO₂, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{c51}R^{d51}, NR^{e51}NR^{d51}R^{d51} NR^{c51}C(O)R^{b51}, NR^{c51}C(O)OR^{a51}, NR^{c51}C(O)NR^{c51}R^{d51}, NR^{e51}S(O)R^{b51}, NR^{c51}S(O)NR^{c51}R^{d51}, NR^{c51}S(O)₂R^{b51}, NR^{c51}S(O)NR^{c51}R^{d51}, S(O)R^{b51}, S(O)NR^{c51}R^{d51}, S(O)₂R^{b51}, and S(O)₂NR^{c51}R^{d51}; and
each R^{a51}, R^{b51}, R^{c51}, and R^{d51} is independently selected from H, C₁₋₆ alkyl, C₁₋₆ haloalkyl, C₂₋₆ alkenyl, and C₂₋₆ alkynyl.

In some embodiments of any of Formulas I and II, R⁴ selected from phenyl, C₃₋₇ cycloalkyl, 5-6 membered heteroaryl, 4-7 membered heterocycloalkyl, OR^{a4}, and NR^{c4}C(O)OR^{a4}, wherein the phenyl, C₃₋₇ cycloalkyl, 5-6 membered heteroaryl, and 4-7 membered heterocycloalkyl of R⁴ are each optionally substituted with 1, 2, 3, or 4 independently selected R^{4A} substituents.

In some embodiments of any of Formulas I and II, R⁴ selected from phenyl, 5-6 membered heteroaryl, 4-7 membered heterocycloalkyl, OR^{a4}, and NR^{c4}C(O)OR^{a4}, wherein the phenyl, 5-6 membered heteroaryl, and 4-7 membered heterocycloalkyl of R⁴ are each optionally substituted with 1, 2, 3, or 4 independently selected R^{4A} substituents.

In some embodiments of any of Formulas I and II, R⁴ is selected from phenyl, tetrahydropyranyl, pyrazolyl, OR^{a4}, and NR^{c4}C(O)OR^{a4}, wherein the phenyl, tetrahydropyranyl, and pyrazolyl of R⁴ are each optionally substituted with 1, 2, 3, or 4 independently selected R^{4A} substituents.

In some embodiments of any of Formulas I and II, R⁴ is selected from phenyl, tetrahydropyranyl, pyrazolyl, OR^{a4}, and NR^{c4}C(O)OR^{a4}, wherein the phenyl, tetrahydropyranyl, and pyrazolyl of R⁴ are each optionally substituted with 1 or 2 independently selected R^{4A} substituents.

In some embodiments of any of Formulas I and II, each R^{a4}, R^{b4}, R^{c4}, and R^{d4} is independently selected from H, C₁₋₆ alkyl, C₁₋₆ haloalkyl, C₂₋₆ alkenyl, and C₂₋₆ alkynyl.

In some embodiments of any of Formulas I and II, each R^{a4} and R^{c4} is independently selected from H, C₁₋₆ alkyl, C₁₋₆ haloalkyl, C₂₋₆ alkenyl, and C₂₋₆ alkynyl.

In some embodiments of any of Formulas I and II, each R^{a4} and R^{c4} is independently selected from H and C₁₋₆ alkyl.

In some embodiments of any of Formulas I and II, each R^{a4} and R^{c4} is independently selected from H and C₁₋₃ alkyl.

In some embodiments of any of Formulas I and II, R⁴ is selected from phenyl, tetrahydropyranyl, pyrazolyl, OR^{a4}, and NR^{c4}C(O)OR^{a4}, wherein the phenyl, tetrahydropyranyl, and pyrazolyl of R⁴ are each optionally substituted with 1, 2, 3, or 4 independently selected R^{4A} substituents; and
each R^{a4} and R^{c4} is independently selected from H, C₁₋₆ alkyl, C₁₋₆ haloalkyl, C₂₋₆ alkenyl, and C₂₋₆ alkynyl.

In some embodiments of any of Formulas I and II, each R^{4A} is independently selected from halo, C₁₋₆ alkyl, C₁₋₆ haloalkyl, C₂₋₆ alkenyl, C₂₋₆ alkynyl, and CN.

In some embodiments of any of Formulas I and II, each R^{4A} is independently selected from C₁₋₆ alkyl, C₁₋₆ haloalkyl, and CN.

In some embodiments of any of Formulas I and II, each R^{4A} is independently selected from C₁₋₆ alkyl and CN.

In some embodiments of any of Formulas I and II, each R^{4A} is independently selected from C₁₋₃ alkyl and CN.

In some embodiments of any of Formulas I and II, each R^{4A} is independently selected from methyl and CN.

In some embodiments of any of Formulas I and II, R⁴ is selected from phenyl, tetrahydropyranyl, pyrazolyl, OR^{a4}, and NR^{c4}C(O)OR^{a4}, wherein the phenyl, tetrahydropyranyl, and pyrazolyl of R⁴ are each optionally substituted with 1 or 2 independently selected R^{4A} substituents; and
each R^{a4} and R^{c4} is independently selected from H and C₁₋₆ alkyl; and
each R^{4A} is independently selected from C₁₋₆ alkyl and CN.

In some embodiments of any of Formulas I and II, R⁵ is selected from triazolyl, pyrazolyl, piperidinyl, tetrahydropyranyl, NHR^{w2}, NHC(O)R^{w2}, NHC(O)OR^{w2}, and

NHS(O)¿R^{w2}, wherein the triazolyl, pyrazolyl, piperidinyl, and tetrahydropyranyl of R⁵ are each optionally substituted with 1, 2, 3, or 4 independently selected R^{5A} substituents.

In some embodiments of any of Formulas I and II, each R^{w2} is independently selected from C₃₋₁₀ cycloalkyl, 5-10 membered heteroaryl, and 4-10 membered heterocycloalkyl, wherein the C₃₋₁₀ cycloalkyl, 5-10 membered heteroaryl, and 4-10 membered heterocycloalkyl of R^{w2} are each optionally substituted with 1, 2, 3, or 4 independently selected R^{5A} substituents.

In some embodiments of any of Formulas I and II, each R^{w2} is independently selected from C₃₋₇ cycloalkyl, 5-6 membered heteroaryl, and 4-7 membered heterocycloalkyl, wherein the C₃₋₇ cycloalkyl, 5-6 membered heteroaryl, and 4-7 membered heterocycloalkyl of R^{w2} are each optionally substituted with 1, 2, 3, or 4 independently selected R^{5A} substituents.

In some embodiments of any of Formulas I and II, each R^{w2} is independently selected from cyclopropyl, cyclobutyl, oxazolyl, and tetrahydropyranyl, wherein the cyclopropyl, cyclobutyl, oxazolyl, and tetrahydropyranyl of R^{w2} are each optionally substituted with 1, 2, 3, or 4 independently selected R^{5A} substituents.

In some embodiments of any of Formulas I and II, each R^{5A} is independently selected from halo, C₁₋₆ alkyl, C₁₋₆ haloalkyl, CN, OR^{a51}, C(O)R^{b51}, and C(O)OR^{a51}.

In some embodiments of any of Formulas I and II, each R^{5A} is independently selected from halo, C₁₋₆ alkyl, and C(O)OR^{a51}.

In some embodiments of any of Formulas I and II, each R^{a51}, R^{b51}, R^{c51}, and R^{d51} is independently selected from H, C₁₋₆ alkyl, C₁₋₆ haloalkyl, C₂₋₆ alkenyl, and C₂₋₆ alkynyl.

In some embodiments of any of Formulas I and II, each R^{a51}, R^{b51}, R^{c51}, and R^{d51} is independently selected from H and C₁₋₆ alkyl.

In some embodiments of any of Formulas I and II, each R^{a51}, R^{b51}, R^{c51}, and R^{d51} is independently selected from H and C₁₋₃ alkyl.

In some embodiments of any of Formulas I and II, each R^{a51} and R^{b51} is independently selected from H, C₁₋₆ alkyl, C₁₋₆ haloalkyl, C₂₋₆ alkenyl, and C₂₋₆ alkynyl.

In some embodiments of any of Formulas I and II, each R^{a51} and R^{b51} is independently selected from H and C₁₋₆ alkyl.

In some embodiments of any of Formulas I and II, each R^{a51} and R^{b51} is independently selected from H and C₁₋₃ alkyl.

In some embodiments of any of Formulas I and II, each R^{5A} is independently selected from halo, C₁₋₆ alkyl, C₁₋₆ haloalkyl, CN, OR^{a51}, C(O)R^{b51}, and C(O)OR^{a51}; and each R^{a51} and R^{b51} is independently selected from H, C₁₋₆ alkyl, C₁₋₆ haloalkyl, C₂₋₆ alkenyl, and C₂₋₆ alkynyl.

In some embodiments of any of Formulas I and II, each R^{5A} is independently selected from halo, C₁₋₆ alkyl, and C(O)OR^{a51}; and
each R^{a51} is independently selected from H and C₁₋₆ alkyl.

In some embodiments of any of Formulas I and II, each R^{5A} is independently selected from fluoromethyl, and ethoxycarbonyl.

In some embodiments of any of Formulas I and II, R⁵ is selected from triazolyl, pyrazolyl, piperidinyl, tetrahydropyranyl, NHR^{w2}, NHC(O)R^{w2}, NHC(O)OR^{w2}, and NHS(O)₂R², wherein the triazolyl, pyrazolyl, piperidinyl, and tetrahydropyranyl of R⁵ are each optionally substituted with 1, 2, 3, or 4 independently selected R^{5A} substituents;
each R^{w2} is independently selected from cyclopropyl, cyclobutyl, oxazolyl, and tetrahydropyranyl, wherein the cyclopropyl, cyclobutyl, oxazolyl, and tetrahydropyranyl of R^{w2} are each optionally substituted with 1, 2, 3, or 4 independently selected R^{5A} substituents;
each R^{5A} is independently selected from halo, C₁₋₆ alkyl, and C(O)OR^{a51}; and
each R^{a51} is independently selected from H and C₁₋₆ alkyl.

In some embodiments, the compound of Formula I is a compound of Formula IIIa, IIIb, IIIc, IIId, Ile, IIIf, IIIg, IIIh, IIIi, or IIIj:

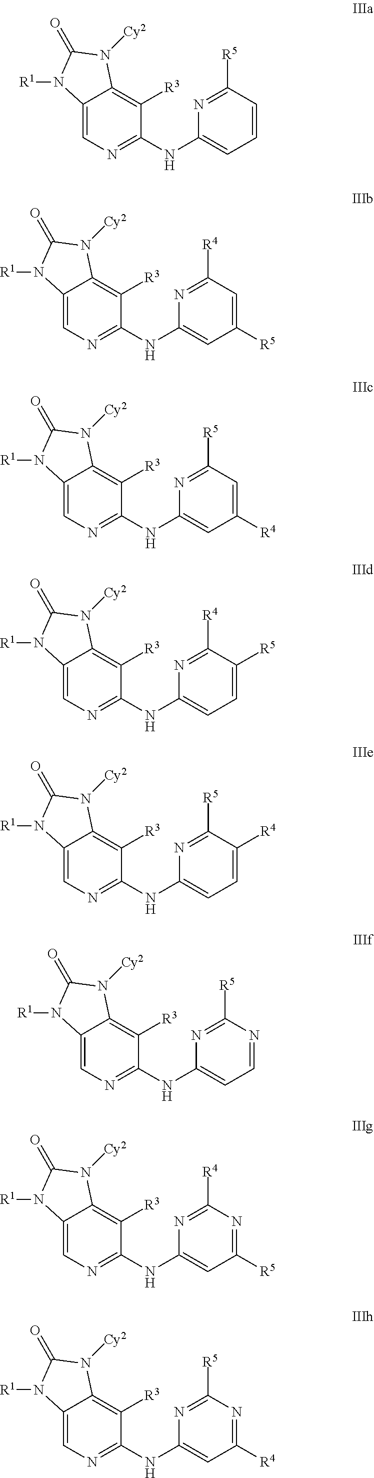

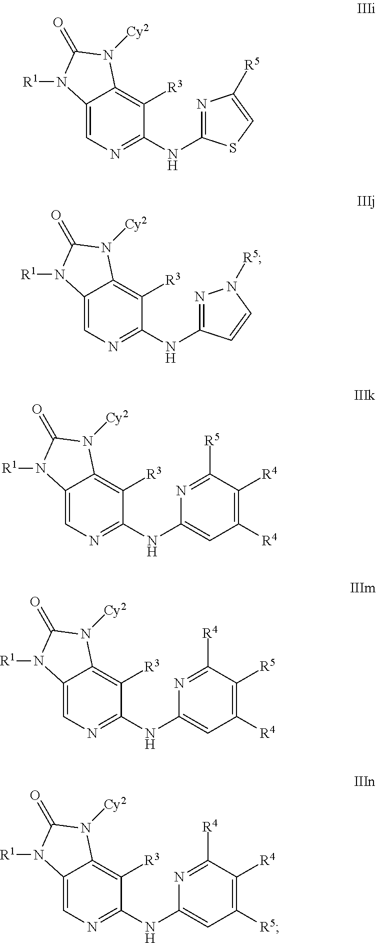

or a pharmaceutically acceptable salt thereof, wherein:
each R⁴ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl of R⁴ are each optionally substituted with 1, 2, 3, or 4 independently selected R^{4A} substituents;
each R^{4A} is independently selected from halo, oxo, C₁₋₆ alkyl, C₁₋₆ haloalkyl, C₂₋₆ alkenyl, C₂₋₆ alkynyl, and CN;
R⁵ is selected from C₁₋₆ alkyl, C₃₋₁₂ cycloalkyl, C₆₋₁₀ aryl, 5-12 membered heteroaryl, tetrahydropyranyl, pyrrolidinyl, piperidinyl, 2,3-dihydrobenzo[b][1,4]dioxinyl, 3,4-dihydro-2H-benzo[b][1,4]oxazinyl, azaspiro[2.4]heptanyl, OR^{w2}, NR^{w2}R^{w3}, C(O)R^{w2}, and C(O)NR^{w2}R^{w3};
wherein the C₁₋₆ alkyl and C₆₋₁₀ aryl of R⁵ are each substituted with 1 R^{w1} substituent and optionally substituted 1 or 2 independently selected R^{5A} substituents; or
the C₆₋₁₀ aryl of R⁵ is substituted with 1 R^{w5} substituent and optionally substituted 1 or 2 independently selected R^{5A} substituents; and
the C₃₋₁₂ cycloalkyl, 5-12 membered heteroaryl, tetrahydropyranyl, pyrrolidinyl, piperidinyl, 2,3-dihydrobenzo[b][1,4]dioxinyl, 3,4-dihydro-2H-benzo[b][1,4]oxazinyl, and azaspiro[2.4]heptanyl of R⁵ are each optionally substituted with 1, 2, 3, or 4 independently selected R^{5A} substituents;
R^{w1} is selected from C₃₋₁₂ cycloalkyl, 4-12 membered heterocycloalkyl, OR^{w4}, and NHR^{w4}, Wherein the C₃₋₁₂ Cycloalkyl and 4-12 Membered Heterocycloalkyl or R^{w1} are each optionally substituted by 1, 2, 3, or 4 independently selected R^{5A} substituents;
each R^{w2} is independently selected from C₃₋₁₂ cycloalkyl, 5-12 membered heteroaryl, and 4-12 membered heterocycloalkyl, wherein the C₃₋₁₂ cycloalkyl, 5-12 membered heteroaryl, and 4-12 membered heterocycloalkyl of R^{w2} are each optionally substituted with 1, 2, 3, or 4 independently selected R^{5A} substituents;
each R^{w3} is independently selected from H, C₁₋₆ alkyl, C₁₋₆ haloalkyl, C₂₋₆ alkenyl, and C₂₋₆ alkynyl;
each R^{w4} is independently selected from C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl;
R^{w5} is C₁₋₆ alkyl which is substituted with CN or OR^{a51};
each R^{5A} is independently selected from halo, oxo, C₁₋₆ alkyl, C₁₋₆ haloalkyl, C₂₋₆ alkenyl, C₂₋₆ alkynyl, CN, OR^{a51}, and NR^{c51}R^{d51}, wherein the C₁₋₆ alkyl, C₂₋₆ alkenyl, and C₂₋₆ alkynyl of R^{5A} are each optionally substituted with OR^{a52} and 1, 2, 3, 4, 5, 6, or 7 independently selected halo groups; and
each R^{a51}, R^{c51}, and R^{d51} is independently selected from H, C₁₋₆ alkyl, C₁₋₆ haloalkyl, C₂₋₆ alkenyl, and C₂₋₆ alkynyl; and
each R^{a52} is independently selected from H and C₁₋₆ alkyl.

In some embodiments of the previous embodiment:
each R⁴ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl of R⁴ are each optionally substituted with 1, 2, 3, or 4 independently selected R^{4A} substituents;
each R^{4A} is independently selected from halo, oxo, C₁₋₆ alkyl, C₁₋₆ haloalkyl, C₂₋₆ alkenyl, C₂₋₆ alkynyl, and CN;
R⁵ is selected from C₁₋₆ alkyl, C₃₋₁₂ cycloalkyl, C₆₋₁₀ aryl, 5-12 membered heteroaryl, pyrrolidinyl, piperidinyl, 2,3-dihydrobenzo[b][1,4]dioxinyl, 3,4-dihydro-2H-benzo[b][1,4]oxazinyl, azaspiro[2.4]heptanyl, OR^{w2}, NR^{w2}R^{w3}, C(O)R^{w2}, and C(O)NR^{w2}R^{w3};
wherein the C₁₋₆ alkyl and C₆₋₁₀ aryl of R⁵ are each substituted with 1 R^{w1} substituent and optionally substituted 1 or 2 independently selected R^{5A} substituents; or
the C₆₋₁₀ aryl of R⁵ is substituted with 1 R^{w5} substituent and optionally substituted 1 or 2 independently selected R^{5A} substituents; and
the C₃₋₁₂ cycloalkyl, 5-12 membered heteroaryl, pyrrolidinyl, piperidinyl, 2,3-dihydrobenzo[b][1,4]dioxinyl, 3,4-dihydro-2H-benzo[b][1,4]oxazinyl, and azaspiro[2.4]heptanyl of R⁵ are each optionally substituted with 1, 2, 3, or 4 independently selected R^{5A} substituents;
R^{w1} is selected from C₃₋₁₂ cycloalkyl, 4-12 membered heterocycloalkyl, OR^{w4}, and NHR^{w4}, wherein the C₃₋₁₂ cycloalkyl and 4-12 membered heterocycloalkyl or R^{w1} are each optionally substituted by 1, 2, 3, or 4 independently selected R^{5A} substituents;
each R^{w2} is independently selected from C₃₋₁₂ cycloalkyl, 5-12 membered heteroaryl, and 4-12 membered heterocycloalkyl, wherein the C₃₋₁₂ cycloalkyl, 5-12 membered heteroaryl, and 4-12 membered heterocycloalkyl of R^{w2} are each optionally substituted with 1, 2, 3, or 4 independently selected R^{5A} substituents;
each R^{w3} is independently selected from H, C₁₋₆ alkyl, C₁₋₆ haloalkyl, C₂₋₆ alkenyl, and C₂₋₆ alkynyl;
each R^{w4} is independently selected from C₁₋₆ haloalkyl, C₂₋₆ alkenyl, C₂₋₆ alkynyl, C₆-10 aryl, C₃₋₁₀ cycloalkyl, 5-10 membered heteroaryl, and 4-10 membered heterocycloalkyl:
R^{w5} is C₁₋₆ alkyl which is substituted with CN or OR^{a51}.
each R^{5A} is independently selected from halo, oxo, C₁₋₆ alkyl, C₁₋₆ haloalkyl, C₂₋₆ alkenyl, C₂₋₆ alkynyl, CN, OR^{a51}, and NR^{e51}R^{d51}; and
each R^{a51}, R^{e51}, and R^{d51} is independently selected from H, C₁₋₆ alkyl, C₁₋₆ haloalkyl, C₂₋₆ alkenyl, and C₂₋₆ alkynyl.

In some embodiments, the compound of Formula I is a compound of Formula IIIa, IIIb, IIIc, IIId, IIIe, IIIf, IIIg, IIIh, IIIi, or IIIj:

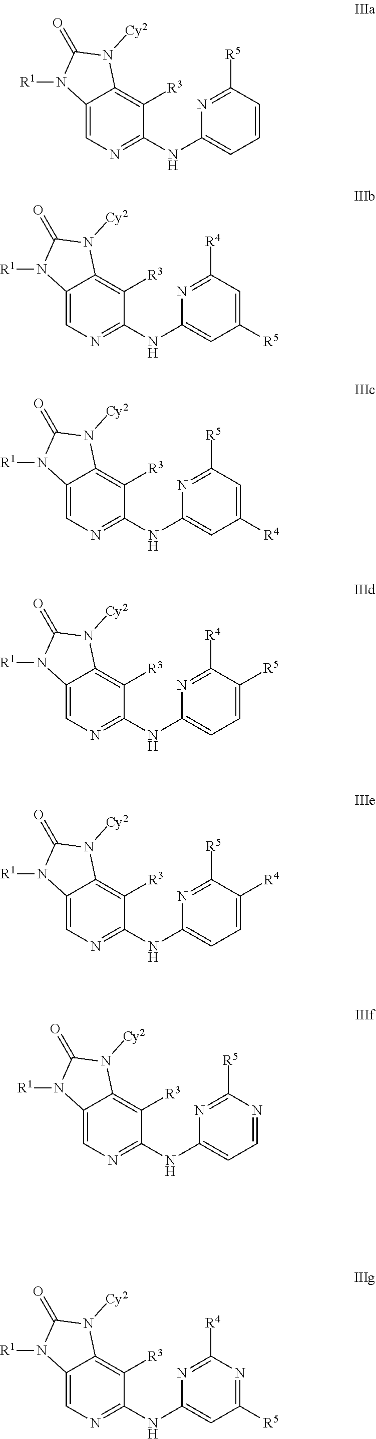

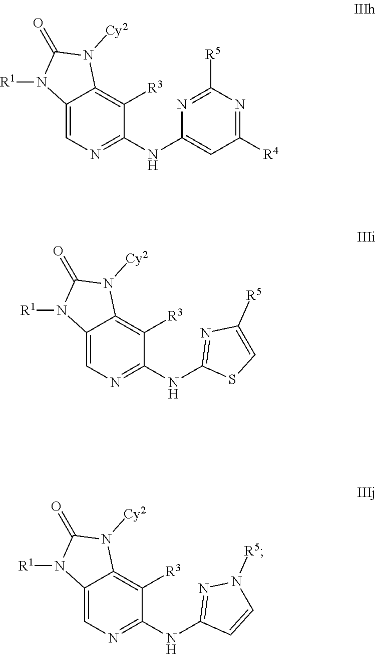

or a pharmaceutically acceptable salt thereof, wherein:
each R⁴ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl of R⁴ are each optionally substituted with 1, 2, 3, or 4 independently selected R^{4A} substituents;
each R^{4A} is independently selected from halo, oxo, C₁₋₆ alkyl, C₁₋₆ haloalkyl, C₂₋₆ alkenyl, C₂₋₆ alkynyl, and CN;
R⁵ is selected from C₁₋₆ alkyl, C₃₋₁₂ cycloalkyl, C₆₋₁₀ aryl, 5-12 membered heteroaryl, pyrrolidinyl, piperidinyl, 2,3-dihydrobenzo[b][1,4]dioxinyl, 3,4-dihydro-2H-benzo[b][1,4]oxazinyl, azaspiro[2.4]heptanyl, OR^{w2}, NR^{w2}R^{w3}, C(O)R^{w2}, and C(O)NR^{w2}R^{w3};
wherein the C₁₋₆ alkyl and C₆₋₁₀ aryl of R⁵ are each substituted with 1 R^{w1} substituent and optionally substituted 1 or 2 independently selected R^{5A} substituents; or
the C₆₋₁₀ aryl of R⁵ is substituted with 1 R^{w5} substituent and optionally substituted 1 or 2 independently selected R^{5A} substituents; and
the C₃₋₁₂ cycloalkyl, 5-12 membered heteroaryl, pyrrolidinyl, piperidinyl, 2,3-dihydrobenzo[b][1,4]dioxinyl, 3,4-dihydro-2H-benzo[b][1,4]oxazinyl, and azaspiro[2.4]heptanyl of R⁵ are each optionally substituted with 1, 2, 3, or 4 independently selected R^{5A} substituents;
R^{w1} is selected from C₃₋₁₂ cycloalkyl, 4-12 membered heterocycloalkyl, OR^{w4}, and NHR^{w4}, wherein the C₃₋₁₂ cycloalkyl and 4-12 membered heterocycloalkyl or R^{w1} are each optionally substituted by 1, 2, 3, or 4 independently selected R^{5A} substituents;
each R^{w2} is independently selected from C₃₋₁₂ cycloalkyl, 5-12 membered heteroaryl, and 4-12 membered heterocycloalkyl, wherein the C₃₋₁₂ cycloalkyl, 5-12 membered heteroaryl, and 4-12 membered heterocycloalkyl of R^{w2} are each optionally substituted with 1, 2, 3, or 4 independently selected R^{5A} substituents;
each R^{w3} is independently selected from H, C₁₋₆ alkyl, C₁₋₆ haloalkyl, C₂₋₆ alkenyl, and C₂₋₆ alkynyl;
each R^{w4} is independently selected from C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl:
R^{w5} is C₁₋₆ alkyl which is substituted with CN or OR^{a51};
each R^{5A} is independently selected from halo, oxo, C₁₋₆ alkyl, C₁₋₆ haloalkyl, C₂₋₆ alkenyl, C₂₋₆ alkynyl, CN, OR^{a51}, and NR^{c51}R^{d51}; and
each R^{a51}, R^{e51}, and R^{d51} is independently selected from H, C₁₋₆ alkyl, C₁₋₆ haloalkyl, C₂₋₆ alkenyl, and C₂₋₆ alkynyl.

In some embodiments, the compound of Formula I is a compound of Formula Va, Vb, Vc, Vd, Vc, Vf, Vg, Vh, VIi, Vj, Vk, Vm, or Vn:

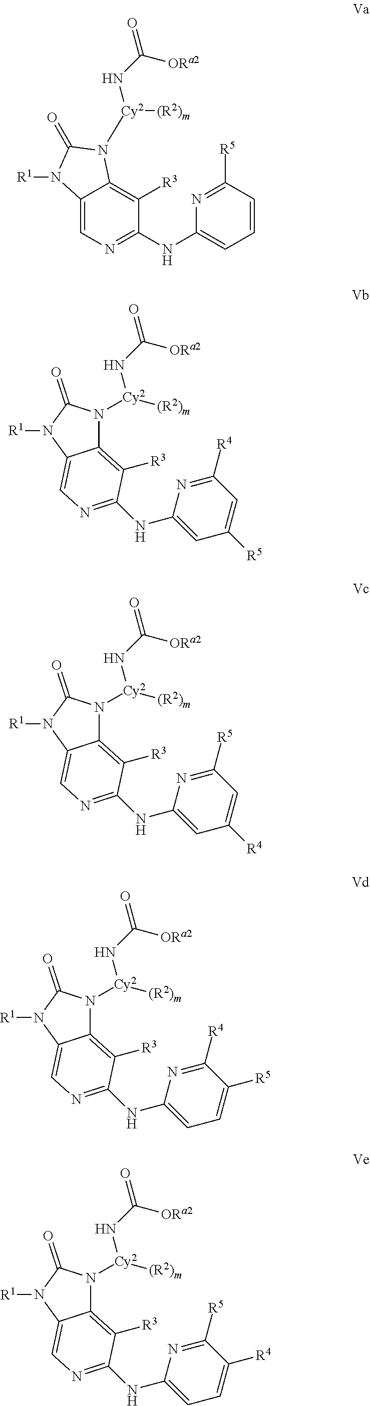

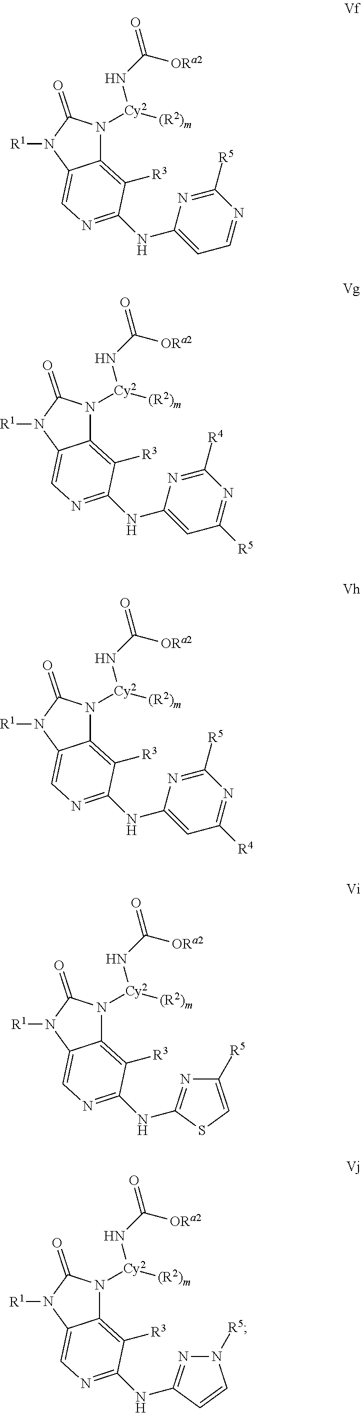

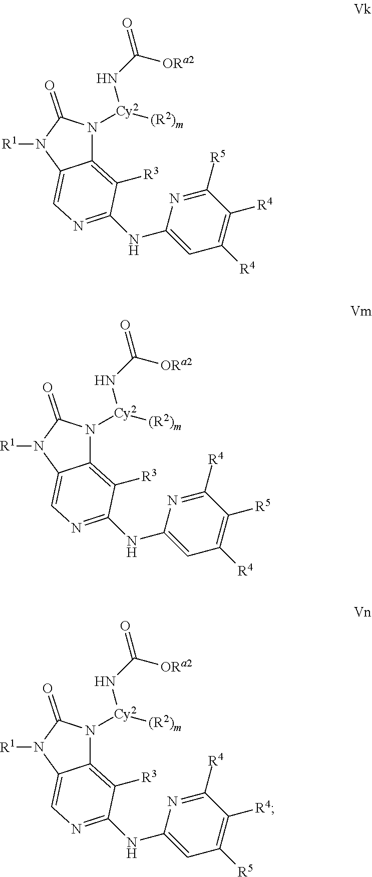

or a pharmaceutically acceptable salt thereof, wherein:

each R⁴ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, (4-10 membered heterocycloalkyl)-C₁₋₆ alkyl-, OR^{a4}, and C(O)R^{b4}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, and (4-10 membered heterocycloalkyl)-C₁₋₆ alkyl- of R⁴ are each optionally substituted with 1, 2, 3, or 4 independently selected R^{4A} substituents;

each R^{a4} and R^{b4} is independently selected from H, C₁₋₆ alkyl, C₁₋₆ haloalkyl, C₂₋₆ alkenyl, C₂₋₆ alkynyl, and 4-6 membered heterocycloalkyl:

each R^{4A} is independently selected from halo, oxo, C₁₋₆ alkyl, C₁₋₆ haloalkyl, C₂₋₆ alkenyl, C₂₋₆ alkynyl, CN, OR^{a41}, and C(O)R^{b41};

each R^{a41} and R^{b41} is independently selected from H, C₁₋₆ alkyl, and C₁₋₆ haloalkyl; R⁵ is selected from C₁₋₆ alkyl, C₃₋₁₂ cycloalkyl, C₆₋₁₀ aryl, 5-12 membered heteroaryl, tetrahydropyranyl, pyrrolidinyl, piperidinyl, 2,3-dihydrobenzo[b][1,4]dioxinyl, 3,4-dihydro-2H-benzo[b][1,4]oxazinyl, azaspiro[2.4]heptanyl, OR^{w2}, NR^{w2}R^{w3}, C(O)R^{w2}, and C(O)NR^{w2}R^{w3};
wherein the C₁₋₆ alkyl and C₆₋₁₀ aryl of R⁵ are each substituted with 1 R^{w1} substituent and optionally substituted 1 or 2 independently selected R^{5A} substituents; or
the C₆₋₁₀ aryl of R⁵ is substituted with 1 R^{w5} substituent and optionally substituted 1 or 2 independently selected R^{5A} substituents; and
the C₃₋₁₂ cycloalkyl, 5-12 membered heteroaryl, tetrahydropyranyl, pyrrolidinyl, piperidinyl, 2,3-dihydrobenzo[b][1,4]dioxinyl, 3,4-dihydro-2H-benzo[b][1,4]oxazinyl, and azaspiro[2.4]heptanyl of R⁵ are each optionally substituted with 1, 2, 3, or 4 independently selected R^{5A} substituents;
R^{w1} is selected from C₃₋₁₂ cycloalkyl, 4-12 membered heterocycloalkyl, OR^{w4}, and NHR^{w4}, wherein the C₃₋₁₂ cycloalkyl and 4-12 membered heterocycloalkyl or R^{w1} are each optionally substituted by 1, 2, 3, or 4 independently selected R^{5A} substituents;
each R^{w2} is independently selected from C₃₋₁₂ cycloalkyl, 5-12 membered heteroaryl, and 4-12 membered heterocycloalkyl, wherein the C₃₋₁₂ cycloalkyl, 5-12 membered heteroaryl, and 4-12 membered heterocycloalkyl of R^{w2} are each optionally substituted with 1, 2, 3, or 4 independently selected R^{5A} substituents;
each R^{w3} is independently selected from H, C₁₋₆ alkyl, C₁₋₆ haloalkyl, C₂₋₆ alkenyl, and C₂₋₆ alkynyl;
each R^{w4} is independently selected from C₁₋₆ haloalkyl, C₂₋₆ alkenyl, C₂₋₆ alkynyl, C₆-10 aryl, C₃₋₁₀ cycloalkyl, 5-10 membered heteroaryl, and 4-10 membered heterocycloalkyl:
R^{w5} is C₁₋₆ alkyl which is substituted with CN or OR^{a51};
each R^{5A} is independently selected from halo, oxo, C₁₋₆ alkyl, C₁₋₆ haloalkyl, C₂₋₆ alkenyl, C₂₋₆ alkynyl, CN, OR^{a51}, and NR^{c51}R^{d51}, wherein the C₁₋₆ alkyl, C₂₋₆ alkenyl, and C₂₋₆ alkynyl of R^{5A} are each optionally substituted with OR^{a52} and 1, 2, 3, 4, 5, 6, or 7 independently selected halo groups;
each R^{a51}, R^{c51}, and R^{d51} is independently selected from H, C₁₋₆ alkyl, C₁₋₆ haloalkyl, C₂₋₆ alkenyl, and C₂₋₆ alkynyl; and
each R^{a52} is independently selected from H and C₁₋₆ alkyl.

In some embodiments of the previous embodiment:
each R⁴ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl of R⁴ are each optionally substituted with 1, 2, 3, or 4 independently selected R^{4A} substituents;
each R^{4A} is independently selected from halo, oxo, C₁₋₆ alkyl, C₁₋₆ haloalkyl, C₂₋₆ alkenyl, C₂₋₆ alkynyl, and CN:
R⁵ is selected from C₁₋₆ alkyl, C₃₋₁₂ cycloalkyl, C₆₋₁₀ aryl, 5-12 membered heteroaryl, pyrrolidinyl, piperidinyl, 2,3-dihydrobenzo[b][1,4]dioxinyl, 3,4-dihydro-2H-benzo[b][1,4]oxazinyl, azaspiro[2.4]heptanyl, OR^{w2}, NR^{w2}R^{w3}, C(O)R^{w2}, and C(O)NR^{w2}R^{w3};
wherein the C₁₋₆ alkyl and C₆₋₁₀ aryl of R⁵ are each substituted with 1 R^{w1} substituent and optionally substituted 1 or 2 independently selected R^{5A} substituents; or
the C₆₋₁₀ aryl of R⁵ is substituted with 1 R^{w5} substituent and optionally substituted 1 or 2 independently selected R^{5A} substituents; and
the C₃₋₁₂ cycloalkyl, 5-12 membered heteroaryl, pyrrolidinyl, piperidinyl, 2,3-dihydrobenzo[b][1,4]dioxinyl, 3,4-dihydro-2H-benzo[b][1,4]oxazinyl, and azaspiro[2.4]heptanyl of R⁵ are each optionally substituted with 1, 2, 3, or 4 independently selected R^{5A} substituents;
R^{w1} is selected from C₃₋₁₂ cycloalkyl, 4-12 membered heterocycloalkyl, OR^{w4}, and NHR^{w4}, wherein the C₃₋₁₂ cycloalkyl and 4-12 membered heterocycloalkyl or R^{w1} are each optionally substituted by 1, 2, 3, or 4 independently selected R^{5A} substituents;
each R² is independently selected from C₃₋₁₂ cycloalkyl, 5-12 membered heteroaryl, and 4-12 membered heterocycloalkyl, wherein the C₃₋₁₂ cycloalkyl, 5-12 membered heteroaryl, and 4-12 membered heterocycloalkyl of R^{w2} are each optionally substituted with 1, 2, 3, or 4 independently selected R^{5A} substituents;
each R^{w3} is independently selected from H, C₁₋₆ alkyl, C₁₋₆ haloalkyl, C₂₋₆ alkenyl, and C₂₋₆ alkynyl;
each R^{w4} is independently selected from C₁₋₆ haloalkyl, C₂₋₆ alkenyl, C₂₋₆ alkynyl, C₆-10 aryl, C₃₋₁₀ cycloalkyl, 5-10 membered heteroaryl, and 4-10 membered heterocycloalkyl;
R^{w5} is C₁₋₆ alkyl which is substituted with CN or OR^{a51};
each R^{5A} is independently selected from halo, oxo, C₁₋₆ alkyl, C₁₋₆ haloalkyl, C₂₋₆ alkenyl, C₂₋₆ alkynyl, CN, OR^{a51}, and NR^{c51}R^{d51}; and
each R^{a51}, R^{e51}, and R^{d51} is independently selected from H, C₁₋₆ alkyl, C₁₋₆ haloalkyl, C₂₋₆ alkenyl, and C₂₋₆ alkynyl.

In some embodiments, the compound of Formula I is a compound of Formula VIa, VIb, VIc, VId, VIe, VIf, VIg, VIh, VIi, VIj, VIk, VIm, or VIn:

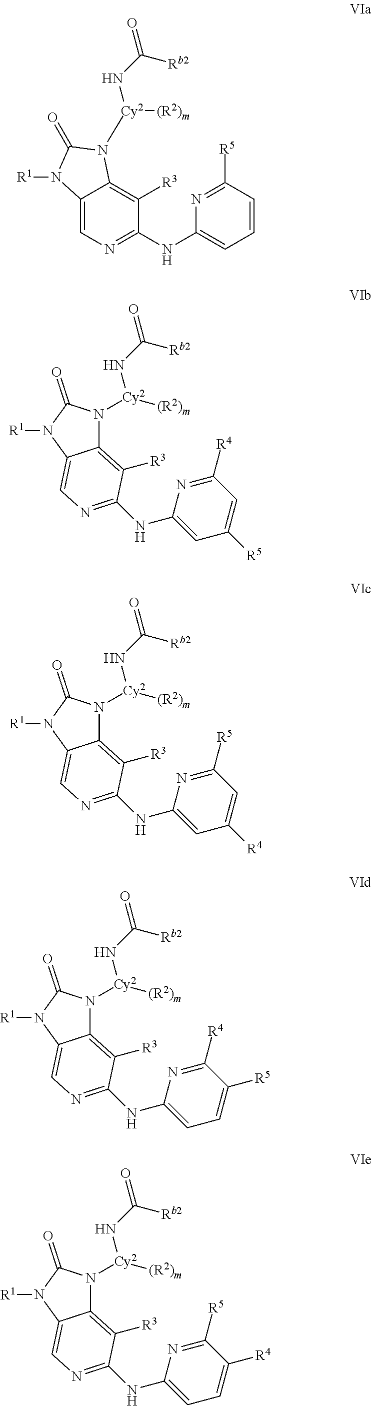

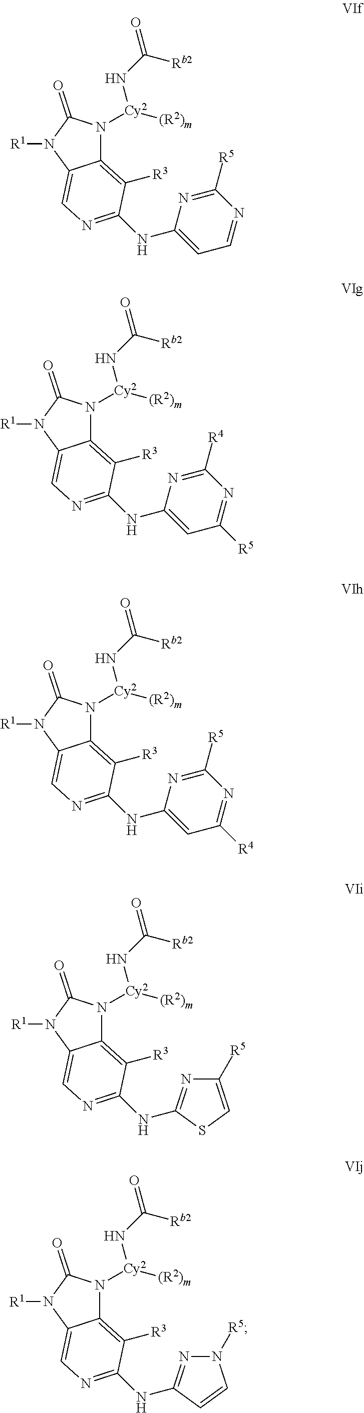

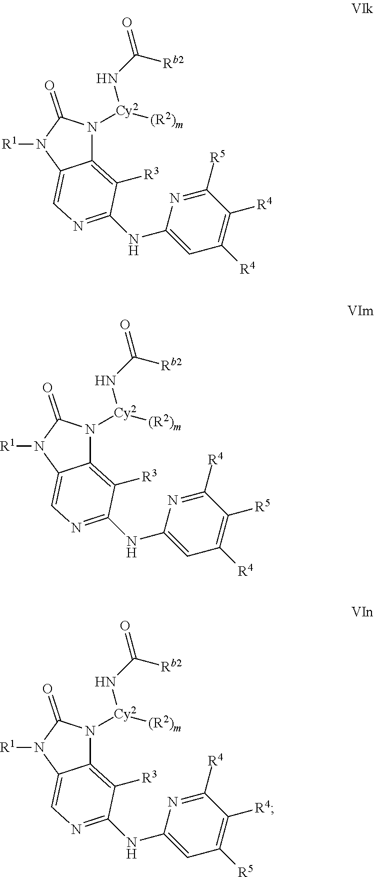

or a pharmaceutically acceptable salt thereof, wherein:

each R⁴ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, (4-10 membered heterocycloalkyl)-C₁₋₆ alkyl-, OR^{a4}, and C(O)R⁴⁴, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, and (4-10 membered heterocycloalkyl)-C₁₋₆ alkyl- of R⁴ are each optionally substituted with 1, 2, 3, or 4 independently selected R^{4A} substituents;
each R^{a4} and R^{b4} is independently selected from H, C₁₋₆ alkyl, C₁₋₆ haloalkyl, C₂₋₆ alkenyl, C₂₋₆ alkynyl, and 4-6 membered heterocycloalkyl:
each R^{4A} is independently selected from halo, oxo, C₁₋₆ alkyl, C₁₋₆ haloalkyl, C₂₋₆ alkenyl, C₂₋₆ alkynyl, CN, OR^{a41}, and C(O)R^{b4}1;
each R^{a41} and R^{b41} is independently selected from H, C₁₋₆ alkyl, and C₁₋₆ haloalkyl;
R⁵ is selected from C₁₋₆ alkyl, C₃₋₁₂ cycloalkyl, C₆₋₁₀ aryl, 5-12 membered heteroaryl, tetrahydropyranyl, pyrrolidinyl, piperidinyl, 2,3-dihydrobenzo[b][1,4]dioxinyl, 3,4-dihydro-2H-benzo[b][1,4]oxazinyl, azaspiro[2.4]heptanyl, OR^{w2}, NR^{w2}R^{w3}, C(O)R^{w2}, and C(O)NR^{w2}R^{w3};
wherein the C₁₋₆ alkyl and C₆₋₁₀ aryl of R⁵ are each substituted with 1 R^{w1} substituent and optionally substituted 1 or 2 independently selected R^{5A} substituents; or
the C₆₋₁₀ aryl of R⁵ is substituted with 1 R^{w5} substituent and optionally substituted 1 or 2 independently selected R^{5A} substituents; and
the C₃₋₁₂ cycloalkyl, 5-12 membered heteroaryl, tetrahydropyranyl, pyrrolidinyl, piperidinyl, 2,3-dihydrobenzo[b][1,4]dioxinyl, 3,4-dihydro-2H-benzo[b][1,4]oxazinyl, and azaspiro[2.4]heptanyl of R⁵ are each optionally substituted with 1, 2, 3, or 4 independently selected R^{5A} substituents;
R^{w1} is selected from C₃₋₁₂ cycloalkyl, 4-12 membered heterocycloalkyl, OR^{w4}, and NHR^{w4}, wherein the C₃₋₁₂ cycloalkyl and 4-12 membered heterocycloalkyl or R^{w1} are each optionally substituted by 1, 2, 3, or 4 independently selected R^{5A} substituents;
each R^{w2} is independently selected from C₃₋₁₂ cycloalkyl, 5-12 membered heteroaryl, and 4-12 membered heterocycloalkyl, wherein the C₃₋₁₂ cycloalkyl, 5-12 membered heteroaryl, and 4-12 membered heterocycloalkyl of R^{w2} are each optionally substituted with 1, 2, 3, or 4 independently selected R^{5A} substituents;
each R^{w3} is independently selected from H, C₁₋₆ alkyl, C₁₋₆ haloalkyl, C₂₋₆ alkenyl, and C₂₋₆ alkynyl;
each R^{w4} is independently selected from C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl;
R^{w5} is C₁₋₆ alkyl which is substituted with CN or OR^{a51};
each R^{5A} is independently selected from halo, oxo, C₁₋₆ alkyl, C₁₋₆ haloalkyl, C₂₋₆ alkenyl, C₂₋₆ alkynyl, CN, OR^{a51}, and NR^{d51}R^{d51}, wherein the C₁₋₆ alkyl, C₂₋₆ alkenyl, and C₂₋₆ alkynyl of R^{5A} are each optionally substituted with OR^{a52} and 1, 2, 3, 4, 5, 6, or 7 independently selected halo groups;
each R^{a51}, R^{c51}, and R^{d51} is independently selected from H, C₁₋₆ alkyl, C₁₋₆ haloalkyl, C₂₋₆ alkenyl, and C₂₋₆ alkynyl; and
each R^{a52} is independently selected from H and C₁₋₆ alkyl.

In some embodiments of the previous embodiment:
each R⁴ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl of R⁴ are each optionally substituted with 1, 2, 3, or 4 independently selected R^{4A} substituents;
each R^{4A} is independently selected from halo, oxo, C₁₋₆ alkyl, C₁₋₆ haloalkyl, C₂₋₆ alkenyl, C₂₋₆ alkynyl, and CN;
R⁵ is selected from C₁₋₆ alkyl, C₃₋₁₂ cycloalkyl, C₆₋₁₀ aryl, 5-12 membered heteroaryl, pyrrolidinyl, piperidinyl, 2,3-dihydrobenzo[b][1,4]dioxinyl, 3,4-dihydro-2H-benzo[b][1,4]oxazinyl, azaspiro[2.4]heptanyl, OR^{w2}, NR^{w2}R^{w3}, C(O)R^{w2}, and C(O)NR^{w2}R^{w3};
wherein the C₁₋₆ alkyl and C₆₋₁₀ aryl of R⁵ are each substituted with 1 R^{w1} substituent and optionally substituted 1 or 2 independently selected R^{5A} substituents; or
the C₆₋₁₀ aryl of R⁵ is substituted with 1 R^{w5} substituent and optionally substituted 1 or 2 independently selected R^{5A} substituents; and
the C₃₋₁₂ cycloalkyl, 5-12 membered heteroaryl, pyrrolidinyl, piperidinyl, 2,3-dihydrobenzo[b][1,4]dioxinyl, 3,4-dihydro-2H-benzo[b][1,4]oxazinyl, and azaspiro[2.4]heptanyl of R⁵ are each optionally substituted with 1, 2, 3, or 4 independently selected R^{5A} substituents;
R^{w1} is selected from C₃₋₁₂ cycloalkyl, 4-12 membered heterocycloalkyl, OR^{w4}, and NHR^{w4}, wherein the C₃₋₁₂ cycloalkyl and 4-12 membered heterocycloalkyl or R¹ are each optionally substituted by 1, 2, 3, or 4 independently selected R^{5A} substituents;
each R^{w2} is independently selected from C₃₋₁₂ cycloalkyl, 5-12 membered heteroaryl, and 4-12 membered heterocycloalkyl, wherein the C₃₋₁₂ cycloalkyl, 5-12 membered heteroaryl, and 4-12 membered heterocycloalkyl of R^{w2} are each optionally substituted with 1, 2, 3, or 4 independently selected R^{5A} substituents;
each R^{w3} is independently selected from H, C₁₋₆ alkyl, C₁₋₆ haloalkyl, C₂₋₆ alkenyl, and C₂₋₆ alkynyl;
each R^{w4} is independently selected from C₁₋₆ haloalkyl, C₂₋₆ alkenyl, C₂₋₆ alkynyl, C₆-10 aryl, C₃₋₁₀ cycloalkyl, 5-10 membered heteroaryl, and 4-10 membered heterocycloalkyl;
R^{w5} is C₁₋₆ alkyl which is substituted with CN or OR^{a51};
each R^{5A} is independently selected from halo, oxo, C₁₋₆ alkyl, C₁₋₆ haloalkyl, C₂₋₆ alkenyl, C₂₋₆ alkynyl, CN, OR^{a51}, and NR^{c51}R^{d51}; and
each R^{a51}, R^{e51}, and R^{d51} is independently selected from H, C₁₋₆ alkyl, C₁₋₆ haloalkyl, C₂₋₆ alkenyl, and C₂₋₆ alkynyl.

In some embodiments of each R⁴ is independently selected from halo, C₁₋₆ alkyl, C₆₋₁₀ aryl, 5-6 membered heteroaryl, 4-10 membered heterocycloalkyl, (4-10 membered heterocycloalkyl)-C₁₋₆ alkyl-, OR^{a4}, and C(O)R^{b4}, wherein the C₁₋₆ alkyl, C₆₋₁₀ aryl, 5-6 membered heteroaryl, 4-10 membered heterocycloalkyl, and (4-10 membered heterocycloalkyl)-C₁₋₆ alkyl- of R⁴ are each optionally substituted with 1, 2, 3, or 4 independently selected R^{4A} substituents; and
each R^{a4} and R^{b4} is independently selected from H, C₁₋₆ alkyl, C₁₋₆ haloalkyl, and 4-6 membered heterocycloalkyl.

In some embodiments of any of Formulas I, IIIa-IIIn, Va-Vn, and VIa-VIn, each R⁴ is independently selected from halo, C₁₋₆ alkyl, phenyl, 5-6 membered heteroaryl, 4-10 membered heterocycloalkyl, (4-6 membered heterocycloalkyl)-C₁₋₆ alkyl-, OR^{a4}, and C(O)R^{b4}, wherein the C₁₋₆ alkyl, phenyl, 5-6 membered heteroaryl, 4-10 membered heterocycloalkyl, and (4-6 membered heterocycloalkyl)-C₁₋₆ alkyl- of R⁴ are each optionally substituted with 1, 2, 3, or 4 independently selected R^{4A} substituents

In some embodiments of any of Formulas I, IIIa-IIIn, Va-Vn, and VIa-Vin, each R^{a4} and R^{b4} is independently selected from C₁₋₆ alkyl and 4-6 membered heterocycloalkyl.

In some embodiments of any of Formulas I, IIIa-IIIn, Va-Vn, and VIa-VIn, each R⁴ is independently selected from halo, C₁₋₆ alkyl, phenyl, 5-6 membered heteroaryl, 4-10 membered heterocycloalkyl, (4-6 membered heterocycloalkyl)-C₁₋₆ alkyl-, OR^{a4}, and C(O)R^{b4}, wherein the C₁₋₆ alkyl, phenyl, 5-6 membered heteroaryl, 4-10 membered heterocycloalkyl, and (4-6 membered heterocycloalkyl)-C₁₋₆ alkyl- of R⁴ are each optionally substituted with 1, 2, 3, or 4 independently selected R^{4A} substituents; and

In some embodiments of any of Formulas I, IIIa-IIIn, Va-Vn, and VIa-Vin, each R^{a4} and R^{b4} is independently selected from C₁₋₆ alkyl and 4-6 membered heterocycloalkyl.

In some embodiments of any of Formulas I, IIIa-IIIn, Va-Vn, and VIa-VIn, each R⁴ is independently selected from halo, C₁₋₆ alkyl, C₆₋₁₀ aryl, and 4-10 membered heterocycloalkyl, wherein the C₁₋₆ alkyl, C₆₋₁₀ aryl, and 4-10 membered heterocycloalkyl of R⁴ are each optionally substituted with 1, 2, 3, or 4 independently selected R^{4A} substituents.

In some embodiments of any of Formulas I, IIIa-IIIn, Va-Vn, and VIa-VIn, each R⁴ is independently selected from halo, C₁₋₆ alkyl, phenyl, and 8-10 membered heterocycloalkyl, wherein the C₁₋₆ alkyl, phenyl, and 8-10 membered heterocycloalkyl of R⁴ are each optionally substituted with 1, 2, 3, or 4 independently selected R^{4A} substituents.

In some embodiments of any of Formulas I, IIIa-IIIn, Va-Vn, and VIa-VIn, each R⁴ is independently selected from halo, C₁₋₆ alkyl, phenyl, and bicyclic 8-10 membered heterocycloalkyl, wherein the C₁₋₆ alkyl, phenyl, and bicyclic 8-10 membered heterocycloalkyl of R⁴ are each optionally substituted with 1, 2, 3, or 4 independently selected R^{4A} substituents.

In some embodiments of any of Formulas I, IIIa-IIIn, Va-Vn, and VIa-VIn, each R⁴ is independently selected from fluoro, methyl, trideuteromethyl, isopropyl, methoxy, morpholinylcarbonyl, phenyl, pyrazolyl, pyridinyl, tetrahydropyranyl, piperidinyl, and 2,3-dihydrobenzo[b][1,4]dioxinyl, wherein the methyl, isopropyl, phenyl, pyrazolyl, piperidinyl, and 2,3-dihydrobenzo[b][1,4]dioxinyl of R⁴ are each optionally substituted with 1, 2, 3, or 4 independently selected R^{4A} substituents.

In some embodiments of any of Formulas I, IIIa-IIIn, Va-Vn, and VIa-VIn, each R⁴ is independently selected from fluoro, methyl, phenyl, and 2,3-dihydrobenzo[b][1,4]dioxinyl, wherein the methyl, phenyl, and 2,3-dihydrobenzo[b][1,4]dioxinyl of R⁴ are each optionally substituted with 1, 2, 3, or 4 independently selected R^{4A} substituents.

In some embodiments of any of Formulas I, IIIa-IIIn, Va-Vn, and VIa-VIn, each R^{4A} is independently selected from C₁₋₆ alkyl, C₁₋₆ haloalkyl, CN, OR^{a41}, and C(O)R^{b41}.

In some embodiments of any of Formulas I, IIIa-IIIn, Va-Vn, and VIa-VIn, each R^{a41} and R^{b41} is independently selected from H and C₁₋₆ alkyl.

In some embodiments of any of Formulas I, IIIa-IIIn, Va-Vn, and VIa-VIn, each R^{4A} is independently selected from C₁₋₆ alkyl, C₁₋₆ haloalkyl, CN, OR^{a41}, and C(O)R^{b41}; and each R^{a41} and R^{b41} is independently selected from H and C₁₋₆ alkyl.

In some embodiments of any of Formulas I, IIIa-IIIn, Va-Vn, and VIa-VIn, each R^{4A} is selected from methyl, difluoromethyl, trifluoromethyl, CN, OH, and methylcarbonyl.

In some embodiments of any of Formulas I, IIIa-IIIn, Va-Vn, and VIa-VIn, each R^{4A} is independently selected from C₁₋₆ alkyl, C₁₋₆ haloalkyl, and CN.

In some embodiments of any of Formulas I, IIIa-IIIn, Va-Vn, and VIa-VIn, each R^{4A} is CN.

In some embodiments of any of Formulas I, IIIa-IIIn, Va-Vn, and VIa-VIn, each R⁴ is independently selected from fluoro, methyl, trideuteromethyl, isopropyl, methoxy, morpholinylcarbonyl, phenyl, pyrazolyl, pyridinyl, tetrahydropyranyl, piperidinyl, and 2,3-dihydrobenzo[b][1,4]dioxinyl, wherein the methyl, isopropyl, phenyl, pyrazolyl, piperidinyl, and 2,3-dihydrobenzo[b][1,4]dioxinyl of R⁴ are each optionally substituted with 1, 2, 3, or 4 independently selected R^{4A} substituents;
each R^{4A} is independently selected from C₁₋₆ alkyl, C₁₋₆ haloalkyl, CN, OR^{a41}, and C(O)R^{b4}1; and
each R^{a41} and R^{b4}1 is independently selected from H and C₁₋₆ alkyl.

In some embodiments of any of Formulas I, IIIa-IIIn, Va-Vn, and VIa-VIn, each R⁴ is independently selected from fluoro, methyl, trideuteromethyl, isopropyl, methoxy, morpholinylcarbonyl, phenyl, pyrazolyl, pyridinyl, tetrahydropyranyl, piperidinyl, and 2,3-dihydrobenzo[b][1,4]dioxinyl, wherein the methyl, isopropyl, phenyl, pyrazolyl, piperidinyl, and 2,3-dihydrobenzo[b][1,4]dioxinyl of R⁴ are each optionally substituted with 1, 2, 3, or 4 independently selected R^{4A} substituents; and
each R^{4A} is selected from methyl, difluoromethyl, trifluoromethyl, CN, OH, and methylcarbonyl.

In some embodiments of any of Formulas I, IIIa-IIIn, Va-Vn, and VIa-VIn, each R⁴ is independently selected from fluoro, methyl, cyanophenyl, and 2,3-dihydrobenzo[b][1,4]dioxinyl.

In some embodiments of any of Formulas I, IIIa-IIIn, Va-Vn, and VIa-VIn, R⁵ is selected from C₁₋₆ alkyl, C₃₋₇ cycloalkyl, phenyl, 5-10 membered heteroaryl, pyrrolidinyl, piperidinyl, 2,3-dihydrobenzo[b][1,4]dioxinyl, 3,4-dihydro-2H-benzo[b][1,4]oxazinyl, azaspiro[2.4]heptanyl, OR^{w2}, NHR^{w2}, C(O)R^{w2}, and C(O)NHR^{w2}, wherein the C₃₋₇ cycloalkyl, 5-10 membered heteroaryl, pyrrolidinyl, piperidinyl, 2,3-dihydrobenzo[b][1,4]dioxinyl, 3,4-dihydro-2H-benzo[b][1,4]oxazinyl, and azaspiro[2.4]heptanyl of R⁵ are each optionally substituted with 1, 2, 3, or 4 independently selected R^{5A} substituents; and
wherein the C₁₋₆ alkyl and phenyl of R⁵ are each substituted with 1 R^{w1} substituent and optionally substituted 1 or 2 independently selected R^{5A} substituents; or
the phenyl of R⁵ is substituted with 1 R^{w5} substituent and optionally substituted 1 or 2 independently selected R^{5A} substituents.

In some embodiments of any of Formulas I, IIIa-IIIn, Va-Vn, and VIa-VIn, R⁵ is selected from —CHOR^{w1}, C₃₋₇ cycloalkyl, -phenyl-R^{w1}, -phenyl-R^{w5}, 5-10 membered heteroaryl, tetrahydropyranyl, pyrrolidinyl, piperidinyl, 2,3-dihydrobenzo[b][1,4]dioxinyl, 3,4-dihydro-2H-benzo[b][1,4]oxazinyl, azaspiro[2.4]heptanyl, OR^{w2}, NHR^{w2}, C(O)R^{w2}, and C(O)NHR^{w2}, wherein the C₃₋₇ cycloalkyl, phenyl, 5-10 membered heteroaryl, tetrahydropyranyl, pyrrolidinyl, piperidinyl, 2,3-dihydrobenzo[b][1,4]dioxinyl, 3,4-dihydro-2H-benzo[b][1,4]oxazinyl, and azaspiro[2.4]heptanyl of R⁵ are each optionally substituted with 1, 2, 3, or 4 independently selected R^{5A} substituents.

In some embodiments of any of Formulas I, IIIa-IIIn, Va-Vn, and VIa-VIn, R⁵ is selected from —CH—R^{w1}, C₃₋₇ cycloalkyl, -phenyl-R=1, -phenyl-R^{w5}, 5-10 membered heteroaryl, pyrrolidinyl, piperidinyl, 2,3-dihydrobenzo[b][1,4]dioxinyl, 3,4-dihydro-2H-benzo[b][1,4]oxazinyl, azaspiro[2.4]heptanyl, OR^{w2}, NHR^{w2}, C(O)R^{w2}, and C(O)NHR^{w2}, wherein the C₃₋₇ cycloalkyl, phenyl, 5-10 membered heteroaryl, pyrrolidinyl, piperidinyl, 2,3-dihydrobenzo[b][1,4]dioxinyl, 3,4-dihydro-2H-benzo[b][1,4]oxazinyl, and azaspiro[2.4]heptanyl of R⁵ are each optionally substituted with 1, 2, 3, or 4 independently selected R^{5A} substituents.

In some embodiments of any of Formulas I, IIIa-IIIn, Va-Vn, and VIa-VIn, R⁵ is selected from —CH—R^{w1}, C₃₋₇ cycloalkyl, -phenyl-R^{w1}, -phenyl-R^{w5}, thiazolyl, thiopheneyl, pyrazolyl, pyridinyl, indazolyl, quinolinyl, tetrahydropyranyl, pyrrolidinyl, piperidinyl, 2,3-dihydrobenzo[b][1,4]dioxinyl, 3,4-dihydro-2H-benzo[b][1,4]oxazinyl, azaspiro[2.4]heptanyl, OR^{w2}, NHR^{w2}, C(O)R^{w2}, and C(O)NHR^{w2}, wherein the C₃₋₇ cycloalkyl, phenyl, pyrazolyl, pyridinyl, indazolyl, quinolinyl, tetrahydropyranyl, pyrrolidinyl, piperidinyl, 2,3-dihydrobenzo[b][1,4]dioxinyl, 3,4-dihydro-2H-benzo[b][1,4]oxazinyl, and azaspiro[2.4]heptanyl of R⁵ are each optionally substituted with 1, 2, 3, or 4 independently selected R^{5A} substituents.

In some embodiments of any of Formulas I, IIIa-IIIn, Va-Vn, and VIa-VIn, R⁵ is selected from —CH₂R^{w1}, C₃₋₇ cycloalkyl, -phenyl-R=1, -phenyl-R^{w5}, thiazolyl, thiopheneyl, pyrazolyl, pyridinyl, indazolyl, quinolinyl, pyrrolidinyl, piperidinyl, 2,3-dihydrobenzo[b][1,4]dioxinyl, 3,4-dihydro-2H-benzo[b][1,4]oxazinyl, azaspiro[2.4]heptanyl, OR^{w2}, NHR^{w2}, C(O)R^{w2}, and C(O)NHR^{w2}, wherein the C₃₋₇ cycloalkyl, phenyl, pyrazolyl, pyridinyl, indazolyl, quinolinyl, pyrrolidinyl, piperidinyl, 2,3-dihydrobenzo[b][1,4]dioxinyl, 3,4-dihydro-2H-benzo[b][1,4]oxazinyl, and azaspiro[2.4]heptanyl of R⁵ are each optionally substituted with 1, 2, 3, or 4 independently selected R^{5A} substituents.

In some embodiments of any of Formulas I, IIIa-IIIn, Va-Vn, and VIa-VIn, R^{w1} is selected from C₃₋₇ cycloalkyl, 4-7 membered heterocycloalkyl, OR^{w4}, and NHR^{w4}, wherein the C₃₋₇ cycloalkyl and 4-7 membered heterocycloalkyl or R^{w1} are each optionally substituted by 1, 2, 3, or 4 independently selected R^{5A} substituents.

In some embodiments of any of Formulas I, IIIa-IIIn, Va-Vn, and VIa-VIn, R^{w1} is selected from C₃₋₇ cycloalkyl, 4-7 membered heterocycloalkyl, OR^{w4}, and NHR^{w4}, wherein the C₃₋₇ cycloalkyl and 4-7 membered heterocycloalkyl or R^{w1} are each optionally substituted by 1 or 2 independently selected R^{5A} substituents.

In some embodiments of any of Formulas I, IIIa-IIIn, Va-Vn, and VIa-VIn, R^{w1} is selected from cyclopropyl, fluoropyrrolidinyl, morpholinyl, OR^{w4}, and NHR^{w4}.

In some embodiments of any of Formulas I, IIIa-IIIn, Va-Vn, and VIa-VIn, R^{w4} is selected from C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl.

In some embodiments of any of Formulas I, IIIa-IIIn, Va-Vn, and VIa-VIn, R^{w4} is selected from C₁₋₆ haloalkyl, C₆₋₁₀ aryl, C₃₋₁₀ cycloalkyl, 5-10 membered heteroaryl, and 4-10 membered heterocycloalkyl.

In some embodiments of any of Formulas I, IIIa-IIIn, Va-Vn, and VIa-VIn, R^{w4} is selected from C₁₋₆ haloalkyl and C₃₋₁₀ cycloalkyl.

In some embodiments of any of Formulas I, IIIa-IIIn, Va-Vn, and VIa-VIn, R^{w4} is selected from C₁₋₆ haloalkyl and C₃₋₇ cycloalkyl.

In some embodiments of any of Formulas I, IIIa-IIIn, Va-Vn, and VIa-VIn, R^{w1} is selected from cyclopropyl, fluoropyrrolidinyl, morpholinyl, OR^{w4}, and NHR^{w4}; and
R^{w4} is selected from C₁₋₆ haloalkyl and C₃₋₁₀ cycloalkyl.

In some embodiments of any of Formulas I, IIIa-IIIn, Va-Vn, and VIa-VIn, R^{w1} is selected from cyclopropyl, fluoropyrrolidinyl, morpholinyl, OR^{w4}, and NHR^{w4}; and
R^{w4} is selected from C₁₋₆ haloalkyl and C₃₋₇ cycloalkyl.

In some embodiments of any of Formulas I, IIIa-IIIn, Va-Vn, and VIa-VIn, R^{w1} is selected from cyclopropyl, fluoropyrrolidinyl, morpholinyl, trifluoromethoxy, and cyclopentylamino.

In some embodiments of any of Formulas I, IIIa-IIIn, Va-Vn, and VIa-VIn, R^{w2} is selected from C₃₋₇ cycloalkyl, bicyclic 8-12 membered heteroaryl, monocyclic 4-7 membered heterocycloalkyl, and bicyclic 8-12 membered heterocycloalkyl, wherein the C₃₋₇ cycloalkyl, bicyclic 8-12 membered heteroaryl, monocyclic 4-7 membered heterocycloalkyl, and bicyclic 8-12 membered heterocycloalkyl of R^{w2} are each optionally substituted with 1, 2, 3, or 4 independently selected R^{5A} substituents.

In some embodiments of any of Formulas I, IIIa-IIIn, Va-Vn, and VIa-VIn, R^{w2} is selected from cyclobutyl, cyclopentyl, cyclohexyl, quinolinyl, tetrahydropyranyl, piperidinyl, morpholinyl, pyrrolidinyl, and octahydro-2H-pyrido[1,2-a]pyrazinyl, wherein the cyclobutyl, cyclopentyl, cyclohexyl, quinolinyl, tetrahydropyranyl, piperidinyl, morpholinyl, pyrrolidinyl, and octahydro-2H-pyrido[1,2-a]pyrazinyl of R^{w2} are each optionally substituted with 1, 2, 3, or 4 independently selected R^{5A} substituents.

In some embodiments of any of Formulas I, IIIa-IIIn, Va-Vn, and VIa-VIn, R^{w5} is C₁₋₆ alkyl which is substituted with CN or OR^{a51}.

In some embodiments of any of Formulas I, IIIa-IIIn, Va-Vn, and VIa-VIn, each R^{a51}, R^{b51}, R^{c51}, and R^{d51} is independently selected from H and C₁₋₆ alkyl.

In some embodiments of any of Formulas I, IIIa-IIIn, Va-Vn, and VIa-VIn, each R^{a51} is independently selected from H and C₁₋₆ alkyl.

In some embodiments of any of Formulas I, IIIa-IIIn, Va-Vn, and VIa-VIn, each R^{a51} is independently selected from H and C₁₋₃ alkyl.

In some embodiments of any of Formulas I, IIIa-IIIn, Va-Vn, and VIa-VIn, each R^{a51} is independently C₁₋₃ alkyl.

In some embodiments of any of Formulas I, IIIa-IIIn, Va-Vn, and VIa-VIn, R^{w5} is C₁₋₆ alkyl which is substituted with CN or OR^{a51}; and
R^{a51} is selected from H and C₁₋₃ alkyl.

In some embodiments of any of Formulas I, IIIa-IIIn, Va-Vn, and VIa-VIn, R^{w5} is selected from cyanomethyl and hydroxymethyl.

In some embodiments of any of Formulas I, IIIa-IIIn, Va-Vn, and VIa-VIn, each R^{5A} is independently selected from each R^{5A} is independently selected from halo, C₁₋₆ alkyl, C₁₋₆ haloalkyl, CN, OR^{a51}, and NR^{c51}R^{d51}, wherein the C₁₋₆ alkyl is optionally substituted with 1, 2, 3, 4, 5, 6, 7, or 8 independently selected R^{5B} substituents.

In some embodiments of any of Formulas I, IIIa-IIIn, Va-Vn, and VIa-VIn, each R^{5A} is independently selected from halo, C₁₋₆ alkyl, C₁₋₆ haloalkyl, CN, OR^{a51}, and NR^{c51}R^{d51}, wherein the C₁₋₆ alkyl of R^{5A} is optionally substituted with 1, 2, 3, or 4 independently selected R^{5B} substituents.

In some embodiments of any of Formulas I, IIIa-IIIn, Va-Vn, and VIa-VIn, each R^{5A} is independently selected from halo, C₁₋₆ alkyl, C₁₋₆ haloalkyl, CN, OR^{a51}, and NR^{c51}R^{d51}, wherein the C₁₋₆ alkyl of R^{5A} is optionally substituted with 1 or 2 independently selected R^{5B} substituents.

In some embodiments of any of Formulas I, IIIa-IIIn, Va-Vn, and VIa-VIn, each R^{5B} is independently selected from halo, C₁₋₆ alkyl, C₁₋₆ haloalkyl, and OH.

In some embodiments of Formulas I, IIIa-IIIn, Va-Vn, and VIa-VIn, each R^{5B} is independently selected from OH and halo.

In some embodiments of any of Formulas I, IIIa-IIIn, Va-Vn, and VIa-VIn, each R^{5B} is OH.

In some embodiments of any of Formulas I, IIIa-IIIn, Va-Vn, and VIa-VIn, each R^{5A} is independently selected from each R^{5A} is independently selected from halo, C₁₋₆ alkyl, C₁₋₆ haloalkyl, CN, OR^{a51}, and NR^{c51}R^{d51}, wherein the C₁₋₆ alkyl is optionally substituted with OH and 1, 2, 3, 4, 5, 6, or 7 independently selected halo groups.

In some embodiments of any of Formulas I, IIIa-IIIn, Va-Vn, and VIa-VIn, each R^{5A} is independently selected from halo, C₁₋₆ alkyl, C₁₋₆ haloalkyl, CN, OR^{a51}, and NR^{c51}R^{d51} wherein the C₁₋₆ alkyl of R^{5A} is optionally substituted OH.

In some embodiments of any of Formulas I, IIIa-IIIn, Va-Vn, and VIa-VIn, each R^{5A} is independently selected from halo, C₁₋₆ alkyl, C₁₋₆ haloalkyl, CN, OR^{a51}, and NR^{e51}R^{d51}

In some embodiments of any of Formulas I, IIIa-IIIn, Va-Vn, and VIa-VIn, each R^{5A} is independently selected from halo, C₁₋₆ alkyl, C₁₋₆ haloalkyl, CN, OR^{a51}, and NR^{c51}R^{d51} wherein the C₁₋₆ alkyl of R^{5A} is optionally substituted with 1 or 2 independently selected R^{5B} substituents; and

each R^{a51}, R^{c51}, and R^{d51} is independently selected from H and C₁₋₆ alkyl.

In some embodiments of any of Formulas I, IIIa-IIIn, Va-Vn, and VIa-VIn, each R^{5A} is independently selected from halo, C₁₋₆ alkyl, C₁₋₆ haloalkyl, CN, OR^{a51}, and NR^{e51}R^{d51} wherein the C₁₋₆ alkyl of R^{5A} is optionally substituted OH; and each R^{a51}, R^{e51}, and R^{d51} is independently selected from H and C₁₋₆ alkyl.

In some embodiments of any of Formulas I, IIIa-IIIn, Va-Vn, and VIa-VIn, each R^{5A} is independently selected from halo, C₁₋₆ alkyl, C₁₋₆ haloalkyl, CN, OR^{a51}, and NR^{c51}R^{d51}; and each R^{a51}, R^{e51}, and R^{d51} is independently selected from H and C₁₋₆ alkyl.

In some embodiments of any of Formulas I, IIIa-IIIn, Va-Vn, and VIa-VIn, each R^{5A} is independently selected from chloro, fluoro, methyl, hydroxyisopropyl, hydroxyhexafluoroisopropyl, difluoromethyl, trifluoromethyl, CN, methoxy, ethoxy, and amino.

In some embodiments of any of Formulas I, IIIa-IIIn, Va-Vn, and VIa-VIn, each R^{5A} is independently selected from chloro, fluoro, methyl, hydroxyisopropyl, difluoromethyl, trifluoromethyl, CN, methoxy, and amino.

In some embodiments, the compound of Formula I is a compound of Formula IVa, IVb, IVc, IVd, IVe, IVf, IVg, IVh, IVi, IVj, IVk, IVm, IVn, IVo, IVp, IVq, IVr, IVs, IVt, IVu, or IVv:

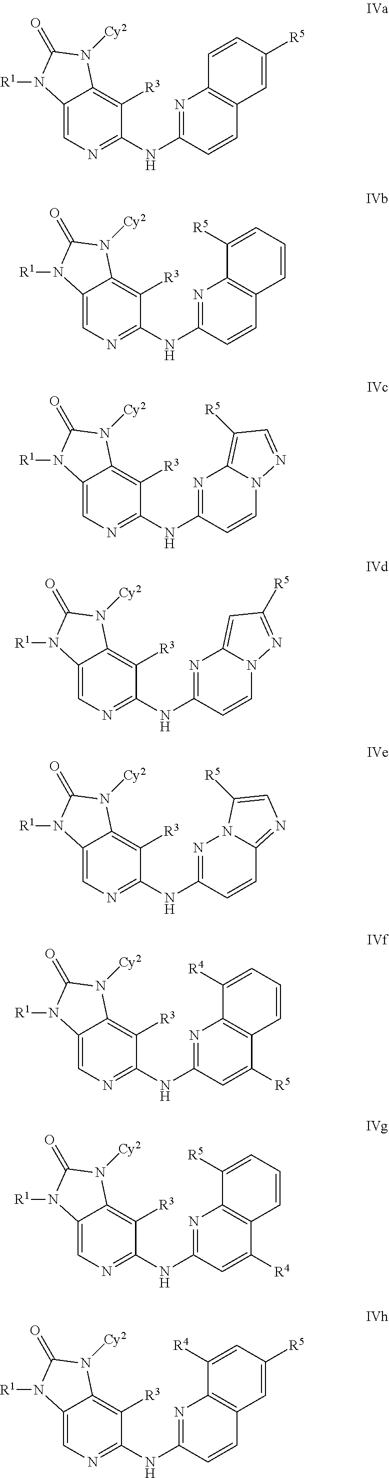

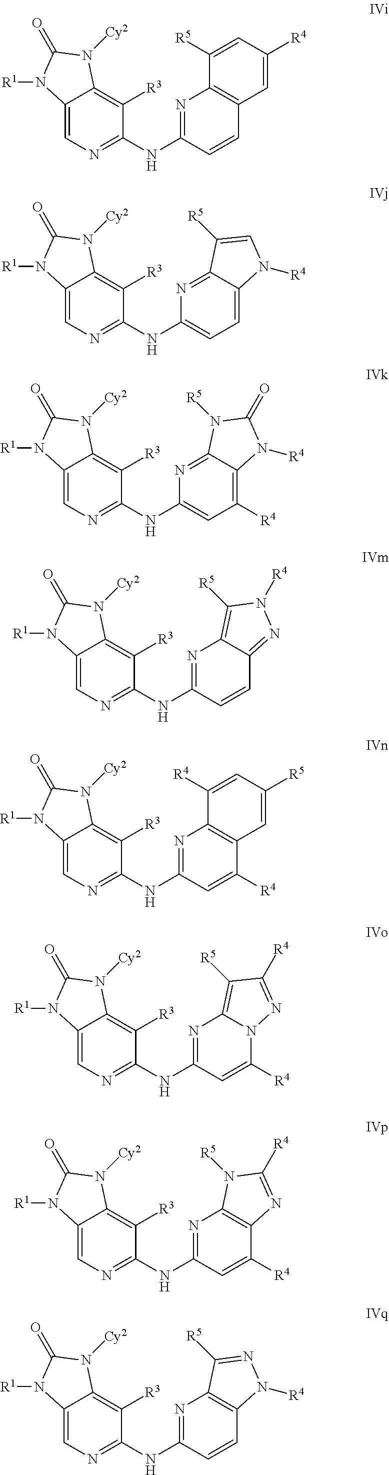

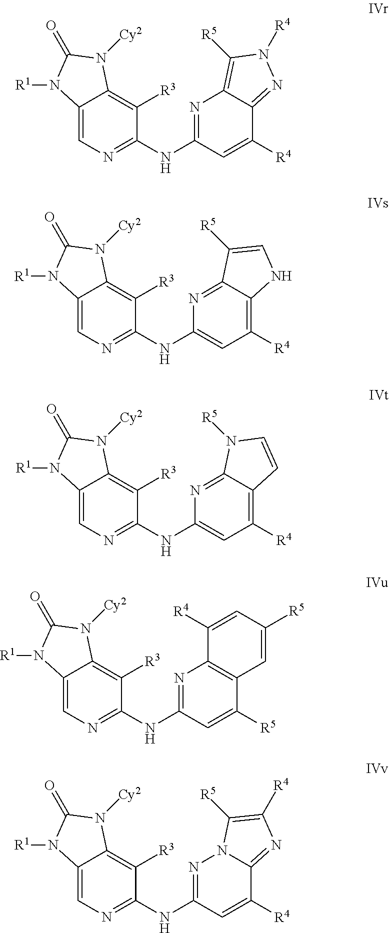

or a pharmaceutically acceptable salt thereof, wherein:
each R⁴ is independently selected from halo, C₁₋₆ alkyl, C₁₋₆ haloalkyl, C₂₋₆ alkenyl, C₂₋₆ alkynyl, C₃₋₇cycloalkyl, 4-7 membered heterocycloalkyl, 5-6 membered heteroaryl, and C₃₋₇ cycloalkyl-C₁₋₆ alkyl-, wherein each C₁₋₆ alkyl, C₂₋₆ alkenyl, C₂₋₆ alkynyl, C₃₋₇ cycloalkyl, 4-7 membered heterocycloalkyl, 5-6 membered heteroaryl, and C₃₋₇ cycloalkyl-C₁₋₆ alkyl- of R⁴ is optionally substituted with 1, 2, 3, or 4 independently selected R^{4A} substituents;
each R^{4A} is independently selected from halo, C₁₋₆ alkyl, C₁₋₆ haloalkyl, C₂₋₆ alkenyl, C₂₋₆ alkynyl, CN, and OR^{a41};
each R^{a41} is independently selected from H, C₁₋₆ alkyl, C₁₋₆ haloalkyl, C₂₋₆ alkenyl, and C₂₋₆ alkynyl;
R⁵ is selected from C₃₋₁₂ cycloalkyl, 5-12 membered heteroaryl, NR^{w2}R^{w3}, and C(O)R^{w2}, wherein the C₃₋₁₂ cycloalkyl and 5-12 membered heteroaryl of R⁵ are each optionally substituted with 1, 2, 3, or 4 independently selected R^{5A} substituents; each R^{w2} is independently selected from C₃₋₁₂ cycloalkyl and 4-12 membered heterocycloalkyl, wherein the C₃₋₁₂ cycloalkyl and 4-12 membered heterocycloalkyl of R^{w2} are each optionally substituted with 1, 2, 3, or 4 independently selected R^{5A} substituents; each R^{w3} is independently selected from H, C₁₋₆ alkyl, C₁₋₆ haloalkyl, C₂₋₆ alkenyl, or C₂₋₆ alkynyl;
each R^{5A} is independently selected from halo, C₁₋₆ alkyl, C₁₋₆ haloalkyl, C₂₋₆ alkenyl, C₂₋₆ alkynyl, CN and OR^{a51}; and
each R^{a51} is independently selected from H, C₁₋₆ alkyl, C₁₋₆ haloalkyl, C₂₋₆ alkenyl, and C₂₋₆ alkynyl.

In some embodiments, the compound of Formula I is a compound of Formula IVa, IVb, IVc, IVd, IVe, IVf, IVg, IVh, IVi, IVj, IVk, IVm, IVn, or IVo:

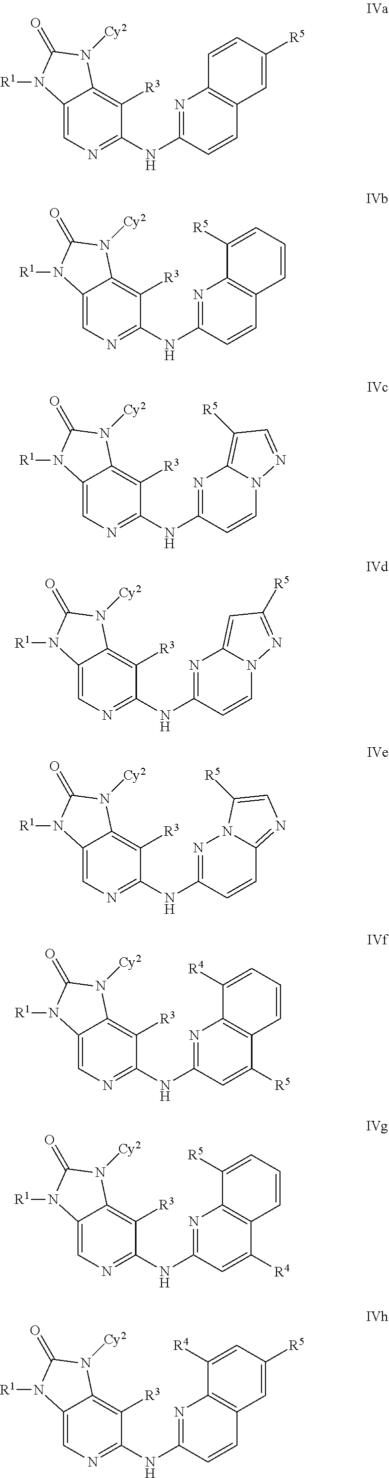

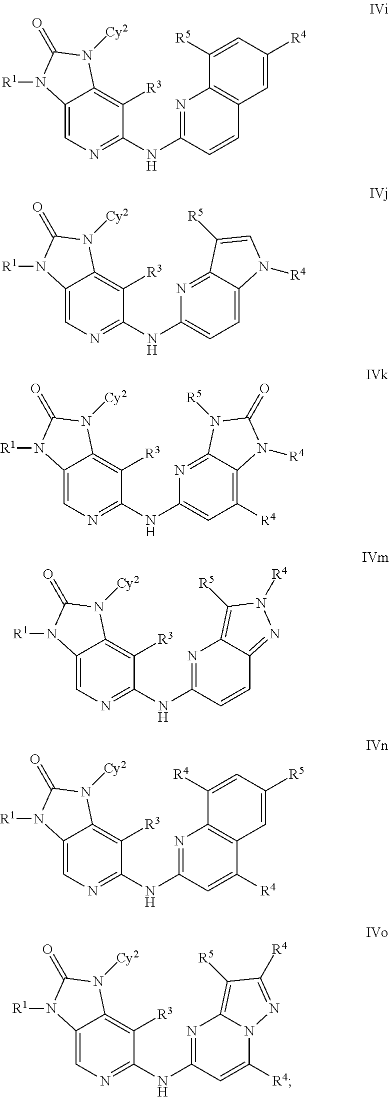

or a pharmaceutically acceptable salt thereof, wherein:

each R⁴ is independently selected from halo, C₁₋₆ alkyl, C₁₋₆ haloalkyl, C₂₋₆ alkenyl, C₂₋₆ alkynyl, C₃₋₇ cycloalkyl, 4-7 membered heterocycloalkyl, 5-6 membered heteroaryl, and C₃₋₇ cycloalkyl-C₁₋₆ alkyl-, wherein each C₁₋₆ alkyl, C₂₋₆ alkenyl, C₂₋₆ alkynyl, C₃₋₇ cycloalkyl, 4-7 membered heterocycloalkyl, 5-6 membered heteroaryl, and C₃₋₇ cycloalkyl-C₁₋₆ alkyl- of R⁴ is optionally substituted with 1, 2, 3, or 4 independently selected R^{4A} substituents;
each R^{4A} is independently selected from halo, C₁₋₆ alkyl, C₁₋₆ haloalkyl, C₂₋₆ alkenyl, C₂₋₆ alkynyl, CN, and OR^{a4}1:
each R^{a41} is independently selected from H, C₁₋₆ alkyl, C₁₋₆ haloalkyl, C₂₋₆ alkenyl, and C₂₋₆ alkynyl;
R⁵ is selected from C₃₋₁₂ cycloalkyl, 5-12 membered heteroaryl, NR^{w2}R^{w3}, and C(O)R^{w2}, wherein the C₃₋₁₂ cycloalkyl and 5-12 membered heteroaryl of R⁵ are each optionally substituted with 1, 2, 3, or 4 independently selected R^{5A} substituents;
each R^{w2} is independently selected from C₃₋₁₂ cycloalkyl and 4-12 membered heterocycloalkyl, wherein the C₃₋₁₂ cycloalkyl and 4-12 membered heterocycloalkyl of R^{w2} are each optionally substituted with 1, 2, 3, or 4 independently selected R^{5A} substituents;
each R^{w3} is independently selected from H, C₁₋₆ alkyl, C₁₋₆ haloalkyl, C₂₋₆ alkenyl, or C₂₋₆ alkynyl;
each R^{5A} is independently selected from halo, C₁₋₆ alkyl, C₁₋₆ haloalkyl, C₂₋₆ alkenyl, C₂₋₆ alkynyl, CN and OR^{a51}; and
each R^{a51} is independently selected from H, C₁₋₆ alkyl, C₁₋₆ haloalkyl, C₂₋₆ alkenyl, and C₂₋₆ alkynyl.

In some embodiments of the previous embodiment:
each R⁴ is independently selected from halo, C₁₋₆ alkyl, C₁₋₆ haloalkyl, C₂₋₆ alkenyl,
C₂₋₆ alkynyl, and 5-6 membered heteroaryl, wherein each C₁₋₆ alkyl, C₂₋₆ alkenyl, C₂₋₆ alkynyl, and 5-6 membered heteroaryl of R⁴ is optionally substituted with 1, 2, 3, or 4 independently selected R^{4A} substituents;
each R^{4A} is independently selected from halo, C₁₋₆ alkyl, C₁₋₆ haloalkyl, C₂₋₆ alkenyl, C₂₋₆ alkynyl, CN, and OR^{a41};
each R^{a41} is independently selected from H, C₁₋₆ alkyl, C₁₋₆ haloalkyl, C₂₋₆ alkenyl, and C₂₋₆ alkynyl;
R⁵ is selected from C₃₋₁₂ cycloalkyl, 5-12 membered heteroaryl, NR^{w2}R^{w3}, and C(O)R^{w2}, wherein the C₃₋₁₂ cycloalkyl and 5-12 membered heteroaryl of R⁵ are each optionally substituted with 1, 2, 3, or 4 independently selected R^{5A} substituents;
each R^{w2} is independently selected from C₃₋₁₂ cycloalkyl and 4-12 membered heterocycloalkyl, wherein the C₃₋₁₂ cycloalkyl and 4-12 membered heterocycloalkyl of R^{w2} are each optionally substituted with 1, 2, 3, or 4 independently selected R^{5A} substituents;
each R^{w3} is independently selected from H, C₁₋₆ alkyl, C₁₋₆ haloalkyl, C₂₋₆ alkenyl, or C₂₋₆ alkynyl;
each R^{5A} is independently selected from halo, C₁₋₆ alkyl, C₁₋₆ haloalkyl, C₂₋₆ alkenyl, C₂₋₆ alkynyl, CN and OR^{a51}; and
each R^{a51} is independently selected from H, C₁₋₆ alkyl, C₁₋₆ haloalkyl, C₂₋₆ alkenyl, and C₂₋₆ alkynyl.

In some embodiments, the compound of Formula I is a compound of Formula IVa, IVb, IVc, IVd, IVe, IVf, IVg, IVh, or IVi:

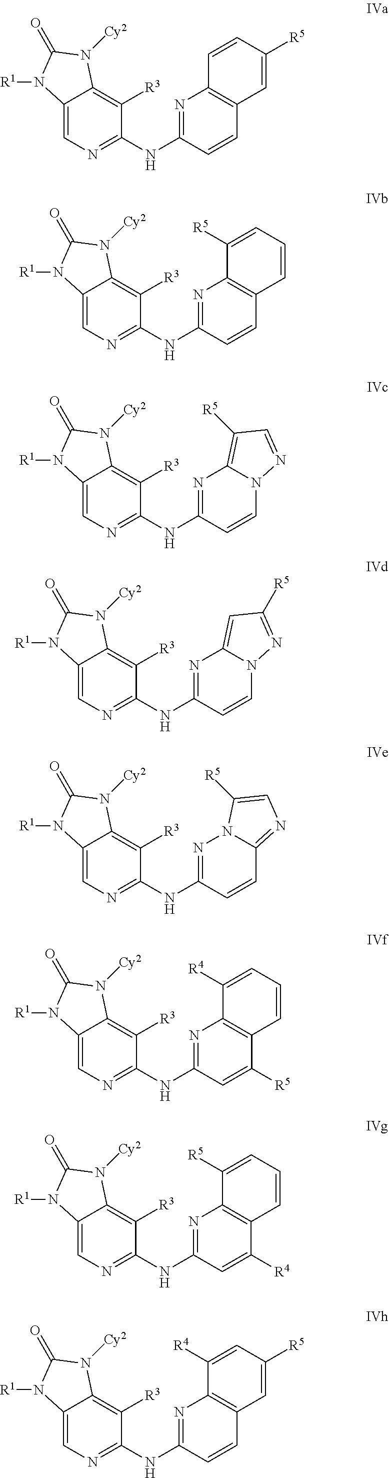

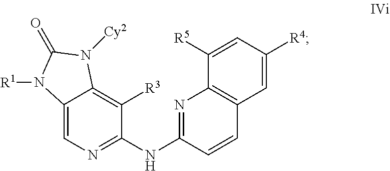

or a pharmaceutically acceptable salt thereof, wherein:
each R⁴ is independently selected from halo, C₁₋₆ alkyl, C₁₋₆ haloalkyl, C₂₋₆ alkenyl, C₂₋₆ alkynyl, C₃₋₇ cycloalkyl, 4-7 membered heterocycloalkyl, 5-6 membered heteroaryl, and C₃₋₇ cycloalkyl-C₁₋₆ alkyl-, wherein each C₁₋₆ alkyl, C₂₋₆ alkenyl, C₂₋₆ alkynyl, C₃₋₇ cycloalkyl, 4-7 membered heterocycloalkyl, 5-6 membered heteroaryl, and C₃₋₇ cycloalkyl-C₁₋₆ alkyl- of R⁴ is optionally substituted with 1, 2, 3, or 4 independently selected R^{4A} substituents;
each R^{4A} is independently selected from halo, C₁₋₆ alkyl, C₁₋₆ haloalkyl, C₂₋₆ alkenyl, C₂₋₆ alkynyl, CN, and OR^{a41};
each R^{a41} is independently selected from H, C₁₋₆ alkyl, C₁₋₆ haloalkyl, C₂₋₆ alkenyl, 10 and C₂₋₆ alkynyl;
R⁵ is selected from C₃₋₁₂ cycloalkyl, 5-12 membered heteroaryl, NR^{w2}R^{w3}, and C(O)R^{w2}, wherein the C₃₋₁₂ cycloalkyl and 5-12 membered heteroaryl of R⁵ are each optionally substituted with 1, 2, 3, or 4 independently selected R^{5A} substituents;
each R^{w2} is independently selected from C₃₋₁₂ cycloalkyl and 4-12 membered heterocycloalkyl, wherein the C₃₋₁₂ cycloalkyl and 4-12 membered heterocycloalkyl of R^{w2} are each optionally substituted with 1, 2, 3, or 4 independently selected R^{5A} substituents;
each R^{w3} is independently selected from H, C₁₋₆ alkyl, C₁₋₆ haloalkyl, C₂₋₆ alkenyl, or C₂₋₆ alkynyl;
each R^{5A} is independently selected from halo, C₁₋₆ alkyl, C₁₋₆ haloalkyl, C₂₋₆ alkenyl, C₂₋₆ alkynyl, CN and OR^{a51}; and
each R^{a51} is independently selected from H, C₁₋₆ alkyl, C₁₋₆ haloalkyl, C₂₋₆ alkenyl, and C₂₋₆ alkynyl.

In some embodiments of the previous embodiment:
each R⁴ is independently selected from halo, C₁₋₆ alkyl, C₁₋₆ haloalkyl, C₂₋₆ alkenyl, and C₂₋₆ alkynyl;
R⁵ is selected from C₃₋₁₂ cycloalkyl, 5-12 membered heteroaryl, NR^{w2}R^{w3}, and C(O)R^{w2}, wherein the C₃₋₁₂ cycloalkyl and 5-12 membered heteroaryl of R⁵ are each optionally substituted with 1, 2, 3, or 4 independently selected R^{5A} substituents;
each R^{w2} is independently selected from C₃₋₁₂ cycloalkyl and 4-12 membered heterocycloalkyl, wherein the C₃₋₁₂ cycloalkyl and 4-12 membered heterocycloalkyl of R^{w2} are each optionally substituted with 1, 2, 3, or 4 independently selected R^{5A} substituents;
each R^{w3} is independently selected from H, C₁₋₆ alkyl, C₁₋₆ haloalkyl, C₂₋₆ alkenyl, or C₂₋₆ alkynyl;
each R^{5A} is independently selected from halo, C₁₋₆ alkyl, C₁₋₆ haloalkyl, C₂₋₆ alkenyl, C₂₋₆ alkynyl, CN and OR^{a51}; and
each R^{a51} is independently selected from H, C₁₋₆ alkyl, C₁₋₆ haloalkyl, C₂₋₆ alkenyl, and C₂₋₆ alkynyl.

In some embodiments, the compound of Formula I is a compound of Formula VIIa, VIIb, VIIc, VIId, VIIe, VIIf, VIIg, VIIh, VIIi, VIIj, VIIk, VIIm, VIIn, or VIIo:

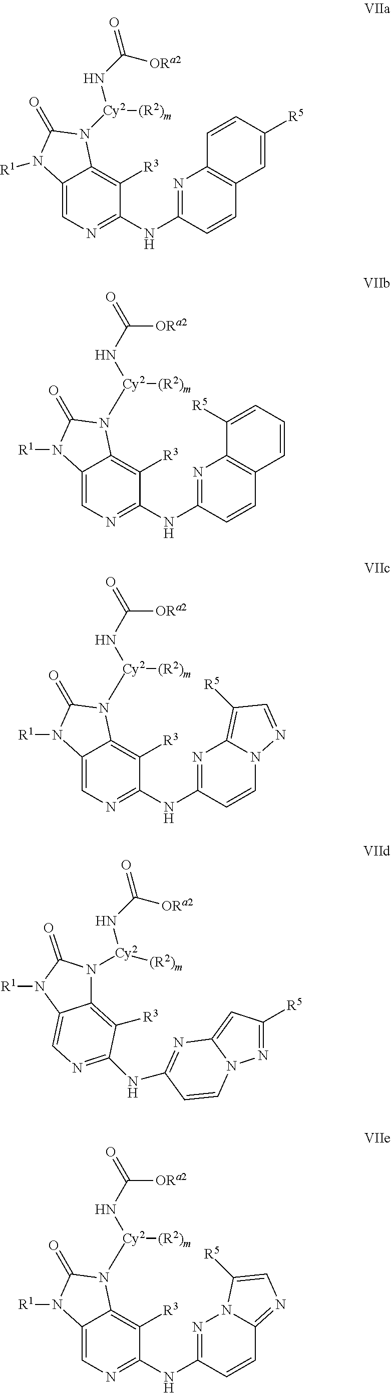

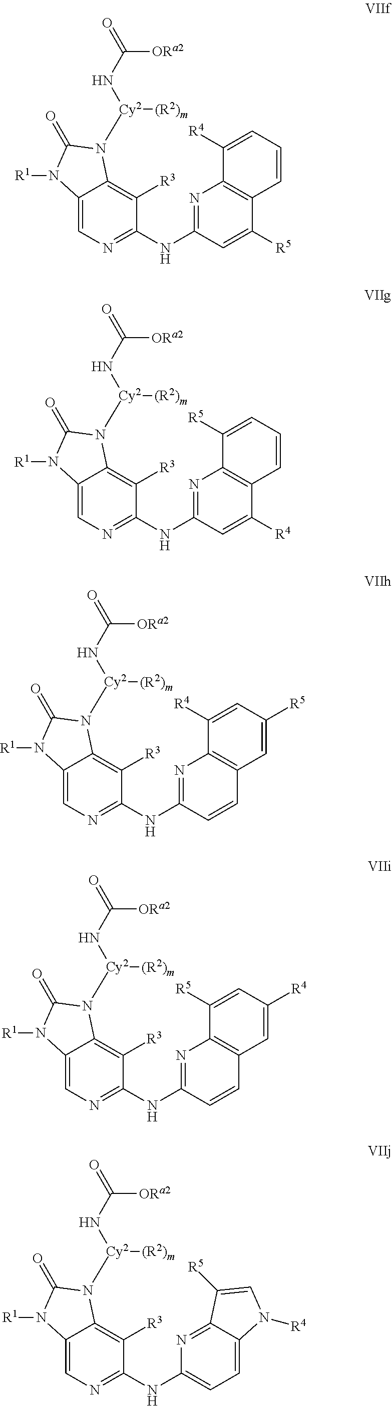

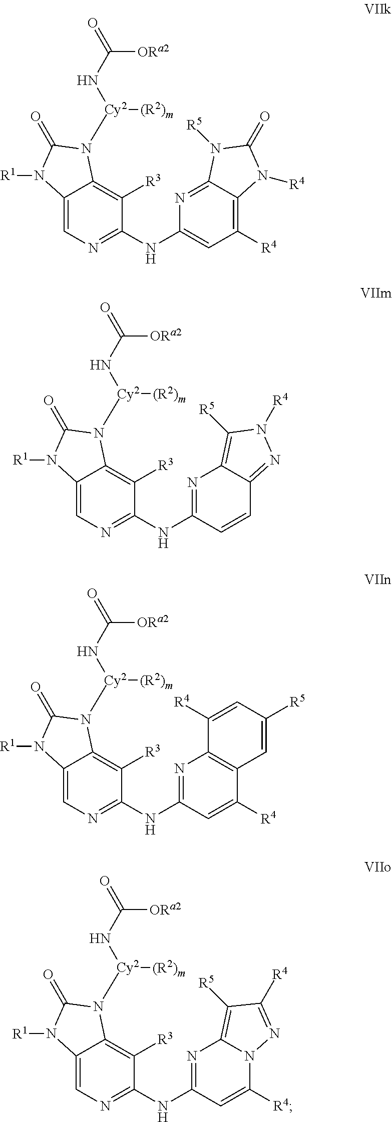

or a pharmaceutically acceptable salt thereof, wherein:
each R⁴ is independently selected from halo, C₁₋₆ alkyl, C₁₋₆ haloalkyl, C₂₋₆ alkenyl, C₂₋₆ alkynyl, C₃₋₇cycloalkyl, 4-7 membered heterocycloalkyl, 5-6 membered heteroaryl, and C₃₋₇cycloalkyl-C₁₋₆ alkyl-, wherein each C₁₋₆ alkyl, C₂₋₆ alkenyl, C₂₋₆ alkynyl, C₃₋₇ cycloalkyl, 4-7 membered heterocycloalkyl, 5-6 membered heteroaryl, and C₃₋₇ cycloalkyl-C₁₋₆ alkyl- of R⁴ is optionally substituted with 1, 2, 3, or 4 independently selected R^{4A} substituents;
each R^{4A} is independently selected from halo, C₁₋₆ alkyl, C₁₋₆ haloalkyl, C₂₋₆ alkenyl, C₂₋₆ alkynyl, CN, and OR^{a41};
each R^{a41} is independently selected from H, C₁₋₆ alkyl, C₁₋₆ haloalkyl, C₂₋₆ alkenyl, and C₂₋₆ alkynyl;
R⁵ is selected from C₃₋₁₂ cycloalkyl, 5-12 membered heteroaryl, NR^{w2}R^{w3}, and C(O)R^{w2}, wherein the C₃₋₁₂ cycloalkyl and 5-12 membered heteroaryl of R⁵ are each optionally substituted with 1, 2, 3, or 4 independently selected R^{5A} substituents;
each R^{w2} is independently selected from C₃₋₁₂ cycloalkyl and 4-12 membered heterocycloalkyl, wherein the C₃₋₁₂ cycloalkyl and 4-12 membered heterocycloalkyl of R^{w2} are each optionally substituted with 1, 2, 3, or 4 independently selected R^{5A} substituents;
each R^{w3} is independently selected from H, C₁₋₆ alkyl, C₁₋₆ haloalkyl, C₂₋₆ alkenyl, or C₂₋₆ alkynyl;
each R^{5A} is independently selected from halo, C₁₋₆ alkyl, C₁₋₆ haloalkyl, C₂₋₆ alkenyl, C₂₋₆ alkynyl, CN and OR^{a51}; and
each R^{a51} is independently selected from H, C₁₋₆ alkyl, C₁₋₆ haloalkyl, C₂₋₆ alkenyl, and C₂₋₆ alkynyl.

In some embodiments of the previous embodiment:
each R⁴ is independently selected from halo, C₁₋₆ alkyl, C₁₋₆ haloalkyl, C₂₋₆ alkenyl, C₂₋₆ alkynyl, and 5-6 membered heteroaryl, wherein each C₁₋₆ alkyl, C₂₋₆ alkenyl, C₂₋₆ alkynyl, and 5-6 membered heteroaryl of R⁴ is optionally substituted with 1, 2, 3, or 4 independently selected R^{4A} substituents;
each R^{4A} is independently selected from halo, C₁₋₆ alkyl, C₁₋₆ haloalkyl, C₂₋₆ alkenyl, C₂₋₆ alkynyl, CN, and OR^{a41};
each R^{a41} is independently selected from H, C₁₋₆ alkyl, C₁₋₆ haloalkyl, C₂₋₆ alkenyl, and C₂₋₆ alkynyl;
R⁵ is selected from C₃₋₁₂ cycloalkyl, 5-12 membered heteroaryl, NR^{w2}R^{w3}, and C(O)R^{w2}, wherein the C₃₋₁₂ cycloalkyl and 5-12 membered heteroaryl of R⁵ are each optionally substituted with 1, 2, 3, or 4 independently selected R^{5A} substituents;
each R^{w2} is independently selected from C₃₋₁₂ cycloalkyl and 4-12 membered heterocycloalkyl, wherein the C₃₋₁₂ cycloalkyl and 4-12 membered heterocycloalkyl of R^{w2} are each optionally substituted with 1, 2, 3, or 4 independently selected R^{5A} substituents;
each R^{w3} is independently selected from H, C₁₋₆ alkyl, C₁₋₆ haloalkyl, C₂₋₆ alkenyl, or C₂₋₆ alkynyl;
each R^{5A} is independently selected from halo, C₁₋₆ alkyl, C₁₋₆ haloalkyl, C₂₋₆ alkenyl, C₂₋₆ alkynyl, CN and OR^{a51}; and
each R^{a51} is independently selected from H, C₁₋₆ alkyl, C₁₋₆ haloalkyl, C₂₋₆ alkenyl, and C₂₋₆ alkynyl.

In some embodiments, the compound of Formula I is a compound of Formula VIIIa, VIIIb, VIIIc, VIIId, VIIIe, VIIIf, VIIIg, VIIIh, VIIIi, VIIIj, VIIIk, VIIIm, VIIIn, or VIIIo:

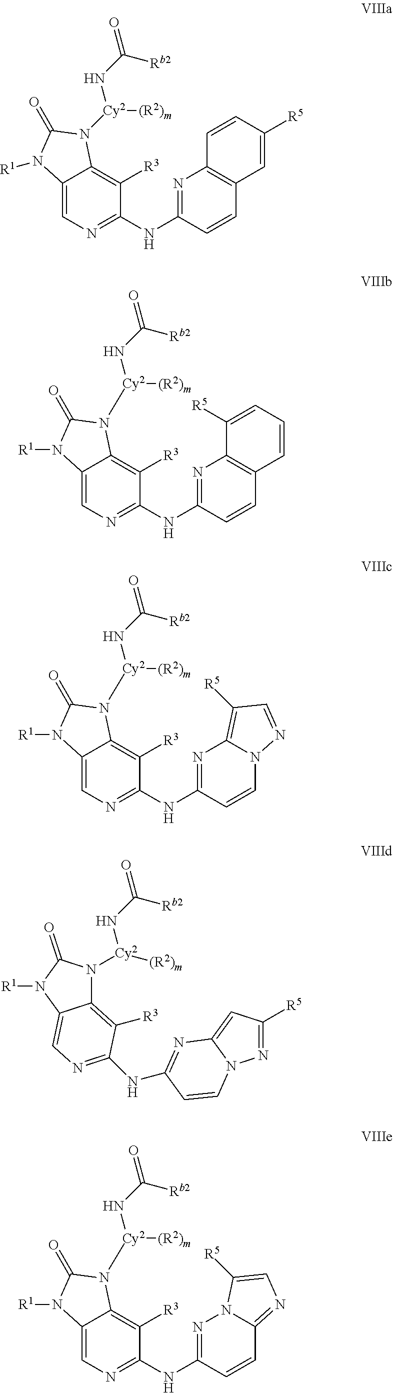

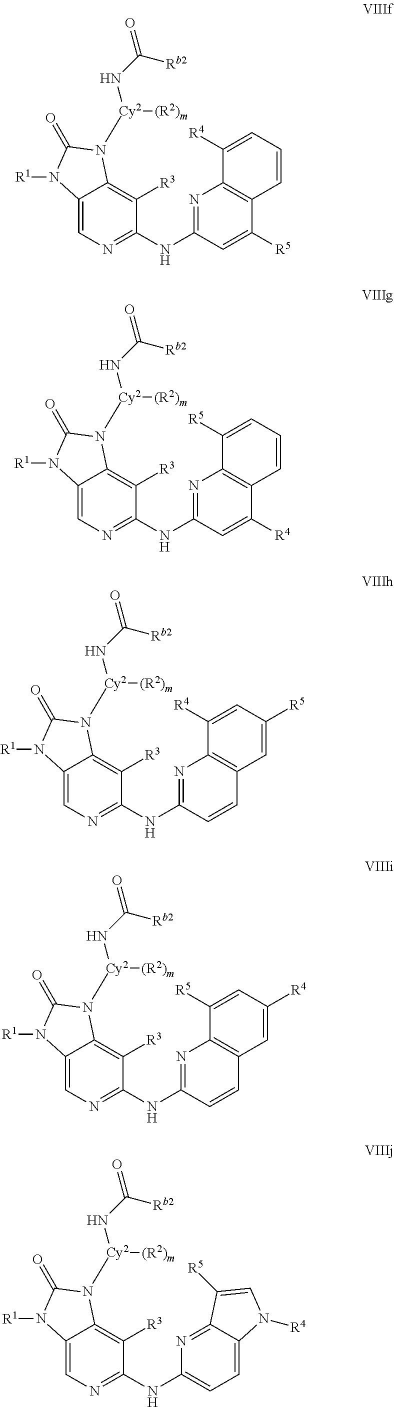

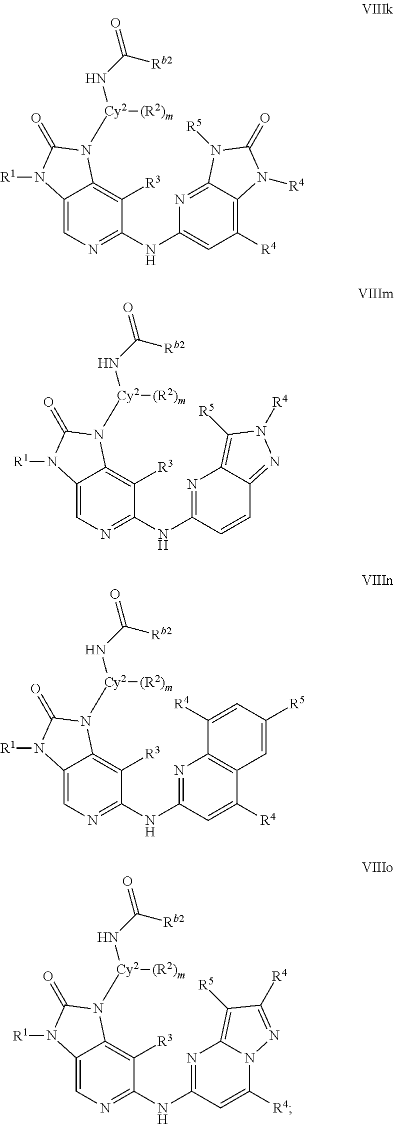

or a pharmaceutically acceptable salt thereof, wherein:
each R⁴ is independently selected from halo, C₁₋₆ alkyl, C₁₋₆ haloalkyl, C₂₋₆ alkenyl, C₂₋₆ alkynyl, C₃₋₇ cycloalkyl, 4-7 membered heterocycloalkyl, 5-6 membered heteroaryl, and C₃₋₇ cycloalkyl-C₁₋₆ alkyl-, wherein each C₁₋₆ alkyl, C₂₋₆ alkenyl, C₂₋₆ alkynyl, C₃₋₇ cycloalkyl, 4-7 membered heterocycloalkyl, 5-6 membered heteroaryl, and C₃₋₇ cycloalkyl-C₁₋₆ alkyl- of R⁴ is optionally substituted with 1, 2, 3, or 4 independently selected R^{4A} substituents;
each R^{4A} is independently selected from halo, C₁₋₆ alkyl, C₁₋₆ haloalkyl, C₂₋₆ alkenyl, C₂₋₆ alkynyl, CN, and OR^{a41};
each R^{a41} is independently selected from H, C₁₋₆ alkyl, C₁₋₆ haloalkyl, C₂₋₆ alkenyl, and C₂₋₆ alkynyl;
R⁵ is selected from C₃₋₁₂ cycloalkyl, 5-12 membered heteroaryl, NR^{w2}R^{w3}, and C(O)R^{w2}, wherein the C₃₋₁₂ cycloalkyl and 5-12 membered heteroaryl of R⁵ are each optionally substituted with 1, 2, 3, or 4 independently selected R^{5A} substituents;
each R^{w2} is independently selected from C₃₋₁₂ cycloalkyl and 4-12 membered heterocycloalkyl, wherein the C₃₋₁₂ cycloalkyl and 4-12 membered heterocycloalkyl of R^{w2} are each optionally substituted with 1, 2, 3, or 4 independently selected R^{5A} substituents;
each R^{w3} is independently selected from H, C₁₋₆ alkyl, C₁₋₆ haloalkyl, C₂₋₆ alkenyl, or C₂₋₆ alkynyl;
each R^{5A} is independently selected from halo, C₁₋₆ alkyl, C₁₋₆ haloalkyl, C₂₋₆ alkenyl, C₂₋₆ alkynyl, CN and OR^{a51}; and
each R^{a51} is independently selected from H, C₁₋₆ alkyl, C₁₋₆ haloalkyl, C₂₋₆ alkenyl, and C₂₋₆ alkynyl.

In some embodiments of the previous embodiment:
each R⁴ is independently selected from halo, C₁₋₆ alkyl, C₁₋₆ haloalkyl, C₂₋₆ alkenyl, C₂₋₆ alkynyl, and 5-6 membered heteroaryl, wherein each C₁₋₆ alkyl, C₂₋₆ alkenyl, C₂₋₆ alkynyl, and 5-6 membered heteroaryl of R⁴ is optionally substituted with 1, 2, 3, or 4 independently selected R^{4A} substituents;
each R^{4A} is independently selected from halo, C₁₋₆ alkyl, C₁₋₆ haloalkyl, C₂₋₆ alkenyl, C₂₋₆ alkynyl, CN, and OR^{a41};
each R^{a41} is independently selected from H, C₁₋₆ alkyl, C₁₋₆ haloalkyl, C₂₋₆ alkenyl, and C₂₋₆ alkynyl;
R⁵ is selected from C₃₋₁₂ cycloalkyl, 5-12 membered heteroaryl, NR^{w2}R^{w3}, and C(O)R^{w2}, wherein the C₃₋₁₂ cycloalkyl and 5-12 membered heteroaryl of R⁵ are each optionally substituted with 1, 2, 3, or 4 independently selected R^{5A} substituents;
each R^{w2} is independently selected from C₃₋₁₂ cycloalkyl and 4-12 membered heterocycloalkyl, wherein the C₃₋₁₂ cycloalkyl and 4-12 membered heterocycloalkyl of R^{w2} are each optionally substituted with 1, 2, 3, or 4 independently selected R^{5A} substituents;
each R^{w3} is independently selected from H, C₁₋₆ alkyl, C₁₋₆ haloalkyl, C₂₋₆ alkenyl, or C₂₋₆ alkynyl;
each R^{5A} is independently selected from halo, C₁₋₆ alkyl, C₁₋₆ haloalkyl, C₂₋₆ alkenyl, C₂₋₆ alkynyl, CN and OR^{a51}; and
each R^{a51} is independently selected from H, C₁₋₆ alkyl, C₁₋₆ haloalkyl, C₂₋₆ alkenyl, and C₂₋₆ alkynyl.

In some embodiments of any of Formulas I, IVa-IVv, VIIa-VIIo, and VIIIa-VIIIo, each R⁴ is independently selected from halo, C₁₋₆ alkyl, C₁₋₆ haloalkyl, C₃₋₇ cycloalkyl, 4-7 membered heterocycloalkyl, 5-6 membered heteroaryl, and C₃₋₇ cycloalkyl-C₁₋₆ alkyl-, wherein each C₁₋₆ alkyl, C₃₋₇cycloalkyl, 4-7 membered heterocycloalkyl, 5-6 membered heteroaryl, and C₃₋₇ cycloalkyl-C₁₋₆ alkyl- of R⁴ is optionally substituted with 1, 2, 3, or 4 independently selected R^{4A} substituents.

In some embodiments of any of Formulas I, IVa-IVv, VIIa-VIIo, and VIIIa-VIIIo, each R^{4A} is independently selected from C₁₋₆ haloalkyl, CN, and OR^{a41}.

In some embodiments of any of Formulas I, IVa-IVv, VIIa-VIIo, and VIIIa-VIIIo, each R^{a41} is independently selected from H and C₁₋₆ alkyl.

In some embodiments of any of Formulas I, IVa-IVv, VIIa-VIIo, and VIIIa-VIIIo, each R⁴ is independently selected from halo, C₁₋₆ alkyl, C₁₋₆ haloalkyl, C₃₋₇cycloalkyl, 4-7 membered heterocycloalkyl, 5-6 membered heteroaryl, and C₃₋₇ cycloalkyl-C₁₋₆ alkyl-, wherein each C₁₋₆ alkyl, C₃₋₇ cycloalkyl, 4-7 membered heterocycloalkyl, 5-6 membered heteroaryl, and C₃₋₇ cycloalkyl-C₁₋₆ alkyl- of R⁴ is optionally substituted with 1, 2, 3, or 4 independently selected R^{4A} substituents;
each R^{4A} is independently selected from C₁₋₆ haloalkyl, CN, and OR^{a41}; and
each R^{a41} is independently selected from H and C₁₋₆ alkyl.

In some embodiments of any of Formulas I, IVa-IVv, VIIa-VIIo, and VIIIa-VIIIo, R⁴ is selected from methyl, ethyl, isopropyl, trifluoromethyl, cyclopropyl, cyclobutyl, cyclohexyl, tetrahydropyranyl, deuterotetrahydropyranyl, pyridinyl, and cyclopropylmethyl, wherein each methyl, ethyl, isopropyl, isobutyl, cyclopropyl, cyclobutyl, cyclohexyl, tetrahydropyranyl, deuterotetrahydropyranyl, pyridinyl, and cyclopropylmethyl of R⁴ is optionally substituted with 1 or 2 R^{4A} substituents independently selected from C₁₋₃ haloalkyl, CN, OH, and C₁₋₃ alkoxy.

In some embodiments of any of Formulas I, IVa-IVv, VIIa-VIIo, and VIIIa-VIIIo, each R⁴ is independently selected from halo, C₁₋₆ alkyl, and C₁₋₆ haloalkyl.

In some embodiments of any of Formulas I, IVa-IVv, VIIa-VIIo, and VIIIa-VIIIo, each R⁴ is independently selected from C₁₋₆ haloalkyl.

In some embodiments of any of Formulas I, IVa-IVv, VIIa-VIIo, and VIIIa-VIIIo, each R⁴ is independently selected from C₁₋₃ haloalkyl.

In some embodiments of any of Formulas I, IVa-IVv, VIIa-VIIo, and VIIIa-VIIIo, each R⁴ is trifluoromethyl.

In some embodiments of any of Formulas I, IVa-IVv, VIIa-VIIo, and VIIIa-VIIIo, R⁵ is selected from C₃₋₇ cycloalkyl, 5-6 membered heteroaryl, NHR^{w2}, and C(O)R^{w2}, wherein the C₃₋₇ cycloalkyl and 5-6 membered heteroaryl of R⁵ are each optionally substituted with 1, 2, 3, or 4 independently selected R^{5A} substituents.

In some embodiments of any of Formulas I, IVa-IVv, VIIa-VIIo, and VIIIa-VIIIo, R⁵ is selected from cyclopropyl, cyclobutyl, cyclohexyl, pyrazolyl, imidazolyl, pyridinyl, NHR^{w2}, and C(O)R^{w2}, wherein the cyclopropyl, cyclobutyl, cyclohexyl, pyrazolyl, imidazolyl, and pyridinyl of R⁵ are each optionally substituted with 1, 2, 3, or 4 independently selected R^{5A} substituents.

In some embodiments of any of Formulas I, IVa-IVv, VIIa-VIIo, and VIIIa-VIIIo, R⁵ is selected from cyclopropyl, pyrazolyl, imidazolyl, NHR^{w2}, and C(O)R^{w2}, wherein the cyclopropyl, pyrazolyl, imidazolyl, of R⁵ are each optionally substituted with 1, 2, 3, or 4 independently selected R^{5A} substituents.

In some embodiments of any of Formulas I, IVa-IVv, VIIa-VIIo, and VIIIa-VIIIo, R^{w2} is selected from C₃₋₇ cycloalkyl, 4-8 membered monocyclic heterocycloalkyl, and 6-10 membered bicyclic heterocycloalkyl, wherein the C₃₋₇ cycloalkyl, 4-8 membered monocyclic heterocycloalkyl, and 6-10 membered bicyclic heterocycloalkyl of R^{w2} are each optionally substituted with 1, 2, 3, or 4 independently selected R^{5A} substituents.

In some embodiments of any of Formulas I, IVa-IVv, VIIa-VIIo, and VIIIa-VIIIo, R^{w2} is selected from cyclopentyl, piperidinyl, morpholinyl, 1,6-diazaspiro[3.3]heptanyl, 3-azabicyclo[3.1.0]hexanyl, 2-oxa-5-azabicyclo[2.2.1]heptanyl, and 2-oxa-5-azabicyclo[4.1.0]heptanyl, wherein the cyclopentyl, piperidinyl, morpholinyl, 1,6-diazaspiro[3.3]heptanyl, 3-azabicyclo[3.1.0]hexanyl, 2-oxa-5-azabicyclo[2.2.1]heptanyl, and 2-oxa-5-azabicyclo[4.1.0]heptanyl of R^{w2} are each optionally substituted with 1, 2, 3, or 4 independently selected R^{5A} substituents.

In some embodiments of any of Formulas I, IVa-IVv, VIIa-VIIo, and VIIIa-VIIIo, each R^{5A} is independently selected from halo, C₁₋₆ alkyl, C₁₋₆ haloalkyl, CN, and OR^{a51}.

In some embodiments of any of Formulas I, IVa-IVv, VIIa-VIIo, and VIIIa-VIIIo, each R^{a51}, R^{b51}, R^{c51}, and R^{d51} is independently selected from H and C₁₋₆ alkyl.

In some embodiments of any of Formulas I, IVa-IVv, VIIa-VIIo, and VIIIa-VIIIo, each R^{a51} is independently selected from H and C₁₋₆ alkyl.

In some embodiments of any of Formulas I, IVa-IVv, VIIa-VIIo, and VIIIa-VIIIo, each R^{a51} is independently selected from H and C₁₋₃ alkyl.

In some embodiments of any of Formulas I, IVa-IVv, VIIa-VIIo, and VIIIa-VIIIo, each R^{5A} is independently selected from halo, C₁₋₆ alkyl, C₁₋₆ haloalkyl, CN, and OR^{a51}; and
each R^{a51} is independently selected from H and C₁₋₆ alkyl.

In some embodiments of any of Formulas I, IVa-IVv, VIIa-VIIo, and VIIIa-VIIIo, each R^{5A} is independently selected from fluoro, methyl, trideuteromethyl, CN, and hydroxy.

In some embodiments of any of Formulas I-VIIIo, R¹ is C₁₋₆ alkyl, which is optionally substituted with 1, 2, or 3 independently selected R^{1A} substituents.

In some embodiments of any of Formulas I-VIIIo, R¹ is C₁₋₆ alkyl.

In some embodiments of any of Formulas I-VIIIo, R¹ is C₁-3 alkyl.

In some embodiments of any of Formulas I-VIIIo, R¹ is methyl or trideuteromethyl.

In some embodiments of any of Formulas I-VIIIo, R¹ is methyl.

In some embodiments of any of Formulas I-VIIIo, R¹ is trideuteromethyl.

In some embodiments of any of Formulas I-VIIIo, Cy² is selected from monocyclic C₃₋₁₀ cycloalkyl, spriocyclic C₆₋₁₀ cycloalkyl, monocyclic 4-7 membered heterocycloalkyl, bicyclic C₆₋₁₀ cycloalkyl, spriocyclic 6-10 membered heterocycloalkyl, and bicyclic 5-10 membered heterocycloalkyl, wherein the monocyclic C₃₋₁₀ cycloalkyl, spriocyclic C₆₋₁₀ cycloalkyl, monocyclic 4-7 membered heterocycloalkyl, bicyclic C₆₋₁₀ cycloalkyl, spriocyclic 6-10 membered heterocycloalkyl, and bicyclic 5-10 membered heterocycloalkyl are each substituted with 1, 2, 3, or 4 independently selected R² substituents.

In some embodiments of any of Formulas I-VIIIo, Cy² is selected from monocyclic C₃₋₁₀ cycloalkyl, spriocyclic C₆₋₁₀ cycloalkyl, monocyclic 4-7 membered heterocycloalkyl, spriocyclic 6-10 membered heterocycloalkyl, and bicyclic 5-10 membered heterocycloalkyl, wherein the monocyclic C₃₋₁₀ cycloalkyl, spriocyclic C₆₋₁₀ cycloalkyl, monocyclic 4-7 membered heterocycloalkyl, spriocyclic 6-10 membered heterocycloalkyl, and bicyclic 5-10 membered heterocycloalkyl are each substituted with 1, 2, 3, or 4 independently selected R² substituents.

In some embodiments of any of Formulas I-VIIIo, Cy² is selected from cyclobutyl, cyclopentyl, deuterocyclopentyl, cyclohexyl, tetrahydrofuranyl, bicyclo[2.2.1]heptanyl, bicyclo[2.2.2]octanyl, spiro[3.3]heptanyl, 3-azabicyclo[3.1.0]hexanyl, 2-azaspiro[3.3]heptanyl, and 2-azabicyclo[2.2.1]heptanyl, wherein the cyclobutyl, cyclopentyl, deuterocyclopentyl, cyclohexyl, tetrahydrofuranyl, bicyclo[2.2.1]heptanyl, bicyclo[2.2.2]octanyl, spiro[3.3]heptanyl, 3-azabicyclo[3.1.0]hexanyl, 2-azaspiro[3.3]heptanyl, and 2-azabicyclo[2.2.1]heptanyl of Cy² are each optionally substituted by 1, 2, 3 or 4 independently selected R² substituents.

In some embodiments of any of Formulas I-VIIIo, Cy² is selected from cyclobutyl, cyclopentyl, deuterocyclopentyl, cyclohexyl, tetrahydrofuranyl, spiro[3.3]heptanyl, 3-azabicyclo[3.1.0]hexanyl, 2-azaspiro[3.3]heptanyl, and 2-azabicyclo[2.2.1]heptanyl, wherein the cyclobutyl, cyclopentyl, deuterocyclopentyl, cyclohexyl, tetrahydrofuranyl, spiro[3.3]heptanyl, 3-azabicyclo[3.1.0]hexanyl, 2-azaspiro[3.3]heptanyl, and 2-azabicyclo[2.2.1]heptanyl of Cy² are each optionally substituted by 1, 2, 3 or 4 independently selected R² substituents.

In some embodiments of any of Formulas I-VIIIo, Cy² is selected from cyclobutyl, cyclopentyl, cyclohexyl, tetrahydrofuranyl, spiro[3.3]heptanyl, 3-azabicyclo[3.1.0]hexanyl, 2-azaspiro[3.3]heptanyl, and 2-azabicyclo[2.2.1]heptanyl, wherein the cyclobutyl, cyclopentyl, cyclohexyl, tetrahydrofuranyl, spiro[3.3]heptanyl, 3-azabicyclo[3.1.0]hexanyl, 2-azaspiro[3.3]heptanyl, and 2-azabicyclo[2.2.1]heptanyl of Cy² are each optionally substituted by 1, 2, 3 or 4 independently selected R² substituents.

In some embodiments of any of Formulas I-VIIIo, Cy² is selected from:

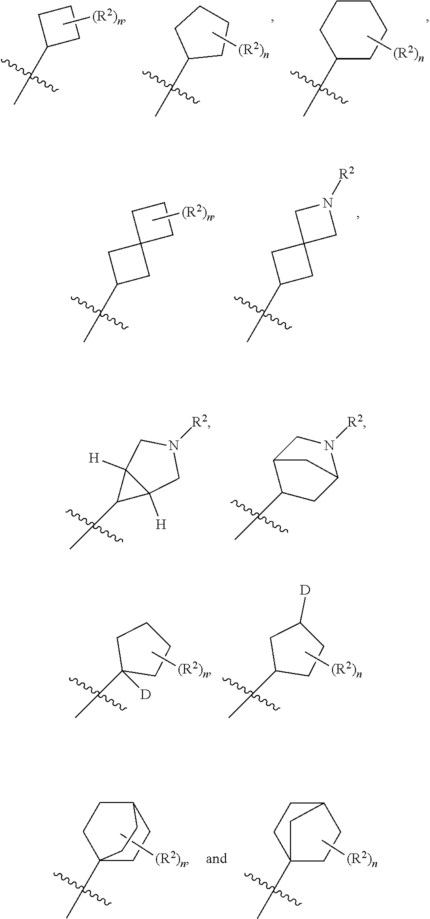

wherein n is 0, 1, or 2.

In some embodiments of any of Formulas I-VIIIo. Cy² is selected from:

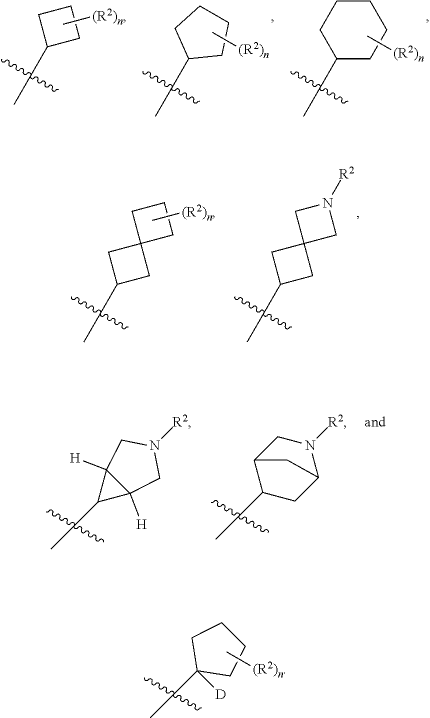

wherein n is 0, 1, or 2.

In some embodiments of any of Formulas I-VIIIo, Cy² is selected from:

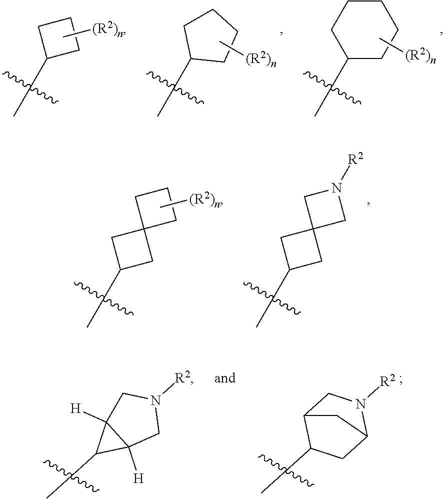

wherein n is 0, 1, or 2.

In some embodiments of any of Formulas I-VIIIo, Cy² is selected from

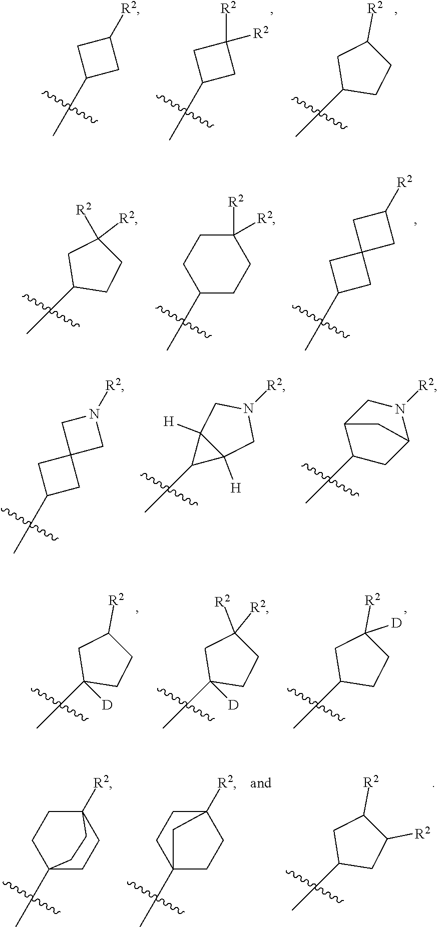

In some embodiments of any of Formulas I-VIIIo, Cy² is selected from:

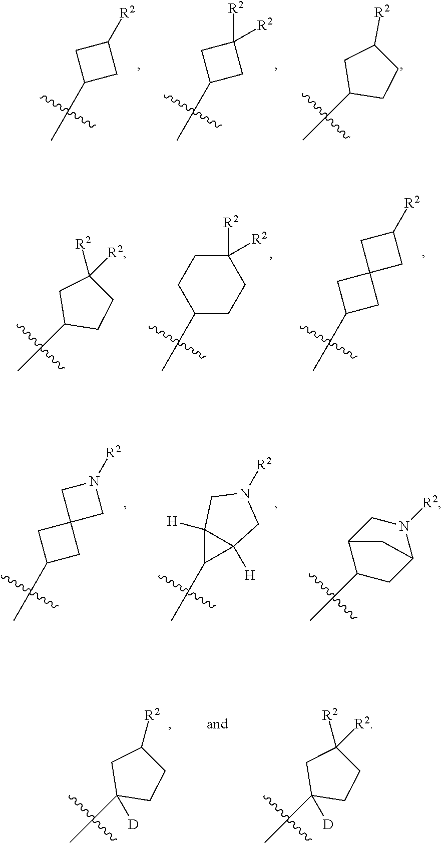

In some embodiments of any of Formulas I-VIIIo. Cy² is selected from:

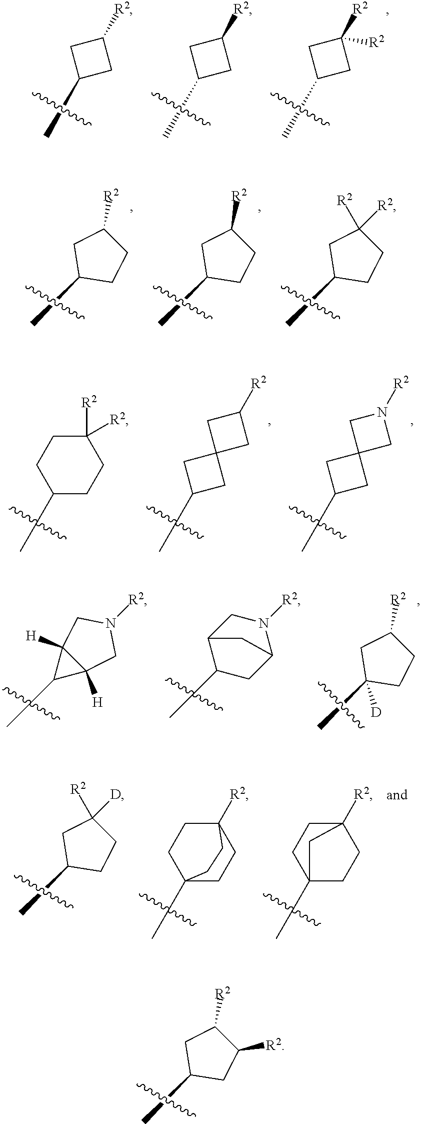

In some embodiments of any of Formulas I-VIIIo, Cy² is selected from:

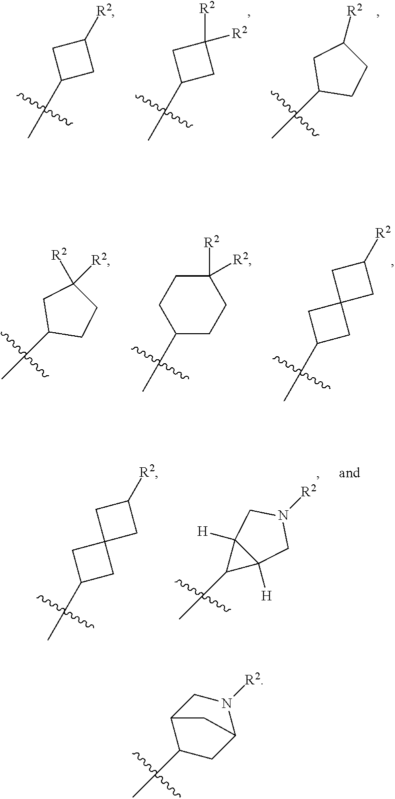

In some embodiments of any of Formulas I-VIIIo, Cy² is selected from:

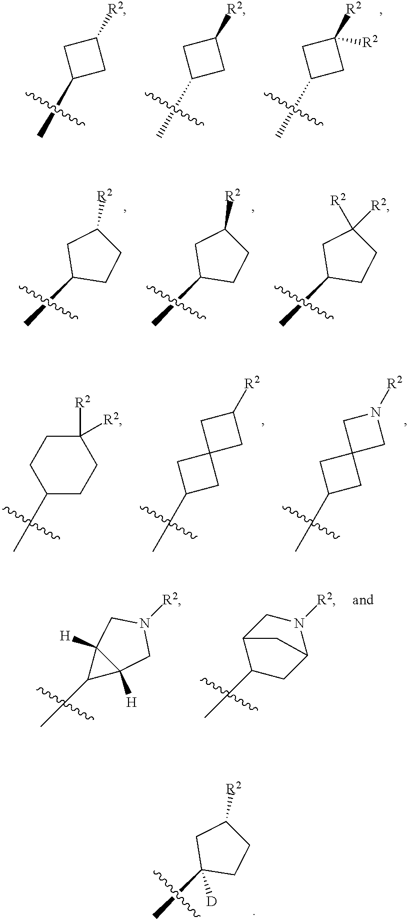

In some embodiments of any of Formulas I-VIIIo, Cy² is selected from:

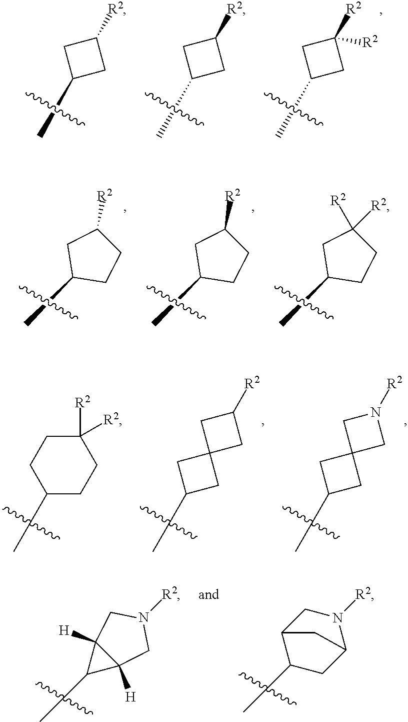

In some embodiments of any of Formulas I-VIIIo, each R² is independently selected from halo, C₁₋₆ alkyl, C₁₋₆ haloalkyl, C₂₋₆ alkenyl, C₂₋₆ alkynyl, CN, OR^{a2}, C(O)R^{b2}, C(O)NR^{c2}R^{d2}, C(O)OR^{a2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR²S(O)₂R^{b2}, S(O)R^{b2}, S(O)NR^{c2}R^{d2}, S(O); R^{b2}, and S(O)—NR^{c2}R^{d2}, wherein the C₁₋₆ alkyl, C₂₋₆ alkenyl, C₂₋₆ alkynyl, of R² are each optionally substituted with 1, 2, 3, or 4 independently selected R^{2A} substituents.

In some embodiments of any of Formulas I-VIIIo, each R² is independently selected from halo, C₁₋₆ alkyl, CN, OR^{a2}, C(O)R^{b2}, C(O)NR^{c2}R^{d2}, C(O)OR^{a2}, NR^{c2}C(O)R^{b2}, NR^{c2}C(O)OR^{a2}, and NR^{c2}S(O)₂R^{b2}, wherein the C₁₋₆ alkyl of R² are each optionally substituted with 1, 2, 3, or 4 independently selected R^{2A} substituents.

In some embodiments of any of Formulas I-VIIIo, each R² is independently selected from halo, C₁₋₆ alkyl, CN, C(O)R^{b2}, C(O)OR^{a2}, NR^{c2}C(O)R^{b2}, NR^{c2}C(O)OR^{a2}, and NR^{c2}S(O)₂R^{b2}, wherein the C₁₋₆ alkyl of R² are each optionally substituted with 1, 2, 3, or 4 independently selected R^{2A} substituents.

In some embodiments of any of Formulas I-VIIIo, each R^{a2}, R^{b2}, R^{c2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl of R^{a2}, R^{b1}, R^{c2}, and R^{d2} are each optionally substituted with 1, 2, 3, or 4 independently selected R^{2A} substituents.

In some embodiments of any of Formulas I-VIIIo, each R^{a2}, R^{b2}, R^{c2}, and R^{d2} is independently selected from H, C₁₋₆ alkyl, C₂₋₆ alkynyl, C₆₋₁₀ aryl, C₃₋₁₀ cycloalkyl, 5-10 membered heteroaryl, and 4-10 membered heterocycloalkyl, wherein the C₁₋₆ alkyl, C₂₋₆ alkynyl, C₆₋₁₀ aryl, C₃₋₁₀ cycloalkyl, 5-10 membered heteroaryl, and 4-10 membered heterocycloalkyl of R^{a2}, R^{b1}, R^{c2}, and R^{d2} are each optionally substituted with 1, 2, 3, or 4 independently selected R^{2A} substituents.

In some embodiments of any of Formulas I-VIIIo, each R^{a2}, R^{b2}, R^{c2}, and R^{d2} is independently selected from H, C₁₋₆ alkyl, C₂₋₆ alkynyl, phenyl, C₃₋₇ cycloalkyl, 5-6 membered heteroaryl, and 4-7 membered heterocycloalkyl, wherein the C₁₋₆ alkyl, phenyl, C₃₋₇ cycloalkyl, 5-6 membered heteroaryl, and 4-7 membered heterocycloalkyl of R^{a2}, R^{b1}, R^{c2}, and R^{d2} are each optionally substituted with 1, 2, 3, or 4 independently selected R^{2A} substituents.

In some embodiments of any of Formulas I-VIIIo, each R^{a2}, R^{b2}, R^{c2}, and R^{d2} is independently selected from H, methyl, trideuteromethyl, ethyl, propynyl, cyclopropyl, cyclobutyl, phenyl, and tetrahydropyranyl, wherein the methyl, ethyl, propynyl, cyclopropyl, cyclobutyl, phenyl, and tetrahydropyranyl of R^{a2}, R^{b1}, R^{c2}, and R^{d2} are each optionally substituted with 1, 2, 3, or 4 independently selected R^{2A} substituents.

In some embodiments of any of Formulas I-VIIIo, each R^{a2}, R^{b2}, R^{c2}, and R^{d2} is independently selected from H, methyl, ethyl, propynyl, cyclopropyl, cyclobutyl, phenyl, and tetrahydropyranyl, wherein the methyl, ethyl, propynyl, cyclopropyl, cyclobutyl, phenyl, and tetrahydropyranyl of R^{a2}, R^{b1}, R^{c2}, and R^{d2} are each optionally substituted with 1, 2, 3, or 4 independently selected R^{2A} substituents.

In some embodiments of any of Formulas I-VIIIo, each R² is independently selected from halo, C₁₋₆ alkyl, CN, C(O)R^{b2}, C(O)OR^{a2}, NR^{c2}C(O)R^{b2}, NR^{c2}C(O)OR^{a2}, and NR^{c2}S(O)₂R^{b2}, wherein the C₁₋₆ alkyl of R² are each optionally substituted with 1, 2, 3, or 4 independently selected R^{2A} substituents; and
each R^{a2}, R^{b2}, R^{c2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl of R^{a2}, R^{b1}, R^{c2}, and R^{d2} are each optionally substituted with 1, 2, 3, or 4 independently selected R^{2A} substituents.

In some embodiments of any of Formulas I-VIIIo, each R² is independently selected from halo, C₁₋₆ alkyl, CN, C(O)R^{b2}, C(O)OR^{a2}, NR^{c2}C(O)R^{b2}, NR^{c2}C(O)OR^{a2}, and NR^{c2}S(O)₂R^{b2}, wherein the C₁₋₆ alkyl of R² are each optionally substituted with 1, 2, 3, or 4 independently selected R^{2A} substituents; and
each R^{a2}, R^{b2}, R^{c2}, and R^{d2} is independently selected from H, C₁₋₆ alkyl, C₂₋₆ alkynyl, C₆₋₁₀ aryl, C₃₋₁₀ cycloalkyl, 5-10 membered heteroaryl, and 4-10 membered heterocycloalkyl, wherein the C₁₋₆ alkyl, C₂₋₆ alkynyl, C₆₋₁₀ aryl, C₃₋₁₀ cycloalkyl, 5-10 membered heteroaryl, and 4-10 membered heterocycloalkyl of R^{a2}, R^{b1}, R^{c2}, and R^{d2} are each optionally substituted with 1, 2, 3, or 4 independently selected R^{2A} substituents.

In some embodiments of any of Formulas I-VIIIo, each R² is independently selected from halo, C₁₋₆ alkyl, CN, C(O)R^{b2}, C(O)OR^{a2}, NR^{c2}C(O)R^{b2}, NR^{c2}C(O)OR^{a2}, and NR^{c2}S(O)₂R^{b2}, wherein the C₁₋₆ alkyl of R² are each optionally substituted with 1, 2, 3, or 4 independently selected R^{2A} substituents; and
each R^{a2}, R^{b2}, R^{c2}, and R^{d2} is independently selected from H, C₁₋₆ alkyl, C₂₋₆ alkynyl, phenyl, C₃₋₇ cycloalkyl, 5-6 membered heteroaryl, and 4-7 membered heterocycloalkyl, wherein the C₁₋₆ alkyl, phenyl, C₃₋₇ cycloalkyl, 5-6 membered heteroaryl, and 4-7 membered heterocycloalkyl of R^{a2}, R^{b1}, R^{c2}, and R^{d2} are each optionally substituted with 1, 2, 3, or 4 independently selected R^{2A} substituents.

In some embodiments of any of Formulas I-VIIIo, each R² is independently selected from halo, C₁₋₆ alkyl, CN, C(O)R^{b2}, C(O)OR^{a2}, NR^{c2}C(O)R^{b2}, NR^{c2}C(O)OR^{a2}, and NR^{c2}S(O)₂R^{b2}, wherein the C₁₋₆ alkyl of R² are each optionally substituted with 1, 2, 3, or 4 independently selected R^{2A} substituents; and
each R^{a2}, R^{b2}, R^{c2}, and R^{d2} is independently selected from H, methyl, ethyl, propynyl, cyclopropyl, cyclobutyl, phenyl, and tetrahydropyranyl, wherein the methyl, ethyl, propynyl, cyclopropyl, cyclobutyl, phenyl, and tetrahydropyranyl of R^{a2}, R^{b1}, R^{c2}, and R^{d2} are each optionally substituted with 1 or 2 independently selected R^{2A} substituents.

In some embodiments of any of Formulas I-VIIIo, each R^{2A} is independently selected from halo, C₁₋₆ alkyl, C₁₋₆ haloalkyl, C₂₋₆ alkenyl, C₂₋₆ alkynyl, CN, and OR^{a21}, wherein the C₁₋₆ alkyl, C₂₋₆ alkenyl, and C₂₋₆ alkynyl of R^{2A} are each optionally substituted with 1, 2, 3, or 4 independently selected R^{2B} substituents.

In some embodiments of any of Formulas I-VIIIo, each R^{2A} is independently selected from halo, C₁₋₆ alkyl, C₁₋₆ haloalkyl, C₂₋₆ alkenyl, C₂₋₆ alkynyl, CN, and OR^{a21}.

In some embodiments of any of Formulas I-VIIIo, each R^{2A} is independently selected from halo and OR^{a21}

In some embodiments of any of Formulas I-VIIIo, each R^{a21}, R^{b21}, R^{e21}, and R^{d21} is independently selected from H, C₁₋₆ alkyl, C₁₋₆ haloalkyl, C₂₋₆ alkenyl, and C₂₋₆ alkynyl.

In some embodiments of any of Formulas I-VIIIo, each R^{a21}, R^{b21}, R^{e21}, and R^{d21} is independently selected from H and C₁₋₆ alkyl.

In some embodiments of any of Formulas I-VIIIo, each R^{a21}, R^{b21}, R^{e21}, and R^{d21} is independently selected from H and C₁₋₃ alkyl.

In some embodiments of any of Formulas I-VIIIo, each R^{a21} is independently selected from H, C₁₋₆ alkyl, C₁₋₆ haloalkyl, C₂₋₆ alkenyl, and C₂₋₆ alkynyl.

In some embodiments of any of Formulas I-VIIIo, each R^{a21} is independently selected from H and C₁₋₆ alkyl.

In some embodiments of any of Formulas I-VIIIo, each R^{a21} is independently selected from H and C₁₋₃ alkyl.

In some embodiments of any of Formulas I-VIIIo, each R^{a21} is independently selected from H and methyl.

In some embodiments of any of Formulas I-VIIIo, each R^{2A} is independently selected from halo and OR^{a21}; and
each R^{a21} is independently selected from H and C₁₋₆ alkyl.

In some embodiments of any of Formulas I-VIIIo, each R^{2A} is independently selected from fluoro, hydroxy, and methoxy.

In some embodiments of any of Formulas I-VIIIo, each R^{2A} is independently selected from fluoro and methoxy.

In some embodiments of any of Formulas I-VIIIo, R³ is selected from H and C₁₋₆ alkyl.

In some embodiments of any of Formulas I-VIIIo, R³ is selected from H and C₁₋₃ alkyl.

In some embodiments of any of Formulas I-VIIIo, R³ is H.

In some embodiments of any of Formulas I-VIIIo:
R¹ is selected from C₁₋₆ alkyl, C₂₋₆ alkenyl, and C₂₋₆ alkynyl;
Cy² is selected from monocyclic C₃₋₁₀ cycloalkyl, spriocyclic C₆₋₁₀ cycloalkyl, monocyclic 4-7 membered heterocycloalkyl, bicyclic C₆₋₁₀ cycloalkyl, spriocyclic 6-10 membered heterocycloalkyl, and bicyclic 5-10 membered heterocycloalkyl, wherein the monocyclic C₃₋₁₀ cycloalkyl, bicyclic C₆₋₁₀ cycloalkyl, spriocyclic C₆₋₁₀ cycloalkyl, monocyclic 4-7 membered heterocycloalkyl, spriocyclic 6-10 membered heterocycloalkyl, and bicyclic 5-10 membered heterocycloalkyl are each substituted with 1, 2, 3, or 4 independently selected R² substituents;
each R² is independently selected from halo, C₁₋₆ alkyl, C₁₋₆ haloalkyl, C₂₋₆ alkenyl, C₂₋₆ alkynyl, CN, OR^{a2}, C(O)R^{b2}, C(O)NR^{c2}R^{d2}, C(O)OR^{a2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O); R^{b2}, S(O)R^{b2}, S(O)NR^{c2}R^{d2}, S(O)₂R^{b2}, and S(O)₂NR^{c2}R^{d2}, wherein the C₁₋₆ alkyl, C₂₋₆ alkenyl, C₂₋₆ alkynyl, of R² are each optionally substituted with 1, 2, 3, or 4 independently selected R^{2A} substituents;
each R^{a2}, R^{b2}, R^{c2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl of R^{a2}, R^{b1}, R^{c2}, and R^{d2} are each optionally substituted with 1, 2, 3, or 4 independently selected R^{2A} substituents;
each R^{2A} is independently selected from halo, C₁₋₆ alkyl, C₁₋₆ haloalkyl, C₂₋₆ alkenyl, C₂₋₆ alkynyl, CN, and OR^{a21};
each R^{a21} is independently selected from H and C₁₋₆ alkyl; and
R³ is selected from H and C₁₋₆ alkyl.

In some embodiments of the previous embodiment, Cy² is selected from monocyclic C₃₋₁₀ cycloalkyl, spriocyclic C₆₋₁₀ cycloalkyl, monocyclic 4-7 membered heterocycloalkyl, spriocyclic 6-10 membered heterocycloalkyl, and bicyclic 5-10 membered heterocycloalkyl, wherein the monocyclic C₃₋₁₀ cycloalkyl, spriocyclic C₆₋₁₀ cycloalkyl, monocyclic 4-7 membered heterocycloalkyl, spriocyclic 6-10 membered heterocycloalkyl, and bicyclic 5-10 membered heterocycloalkyl are each substituted with 1, 2, 3, or 4 independently selected R² substituents;

In some embodiments of any of Formulas I-VIIIo:
R¹ is selected from C₁₋₆ alkyl:
Cy² is selected from monocyclic C₃₋₁₀ cycloalkyl, spriocyclic C₆₋₁₀ Cycloalkyl, monocyclic 4-7 membered heterocycloalkyl, bicyclic C₆₋₁₀ cycloalkyl, spriocyclic 6-10 membered heterocycloalkyl, and bicyclic 5-10 membered heterocycloalkyl, wherein the monocyclic C₃₋₁₀ cycloalkyl, bicyclic C₆₋₁₀ cycloalkyl, spriocyclic C₆₋₁₀ cycloalkyl, monocyclic 4-7 membered heterocycloalkyl, spriocyclic 6-10 membered heterocycloalkyl, and bicyclic 5-10 membered heterocycloalkyl are each substituted with 1, 2, 3, or 4 independently selected R² substituents;
each R² is independently selected from halo, C₁₋₆ alkyl, CN, OR^{a2}, C(O)R^{b2}, C(O)NR²R^{d3}, C(O)OR^{a2}, NR^{c2}C(O)R^{b2}, NR^{c2}C(O)OR^{a2}, and NR^{c2}S(O), R^{b2}, wherein the C₁₋₆ alkyl of R² are each optionally substituted with 1, 2, 3, or 4 independently selected R^{2A} substituents;
each R^{a2}, R^{b2}, R^{c2}, and R^{d2} is independently selected from H, C₁₋₆ alkyl, C₂₋₆ alkynyl, phenyl, C₃₋₇ cycloalkyl, 5-6 membered heteroaryl, and 4-7 membered heterocycloalkyl, wherein the C₁₋₆ alkyl, phenyl, C₃₋₇ cycloalkyl, 5-6 membered heteroaryl, and 4-7 membered heterocycloalkyl of R^{a2}, R^{b1}, R^{c2}, and R^{d2} are each optionally substituted with 1, 2, 3, or 4 independently selected R^{2A} substituents;
each R^{2A} is independently selected from halo, C₁₋₆ alkyl, C₁₋₆ haloalkyl, C₂₋₆ alkenyl, C₂₋₆ alkynyl, CN, and OR^{a21};
each R^{a21} is independently selected from H and C₁₋₆ alkyl; and
R³ is H.

In some embodiments of the previous embodiment:
Cy² is selected from monocyclic C₃₋₁₀ cycloalkyl, spriocyclic C₆₋₁₀ cycloalkyl, monocyclic 4-7 membered heterocycloalkyl, spriocyclic 6-10 membered heterocycloalkyl, and bicyclic 5-10 membered heterocycloalkyl, wherein the monocyclic C₃₋₁₀ cycloalkyl, spriocyclic C₆₋₁₀ cycloalkyl, monocyclic 4-7 membered heterocycloalkyl, spriocyclic 6-10 membered heterocycloalkyl, and bicyclic 5-10 membered heterocycloalkyl are each substituted with 1, 2, 3, or 4 independently selected R² substituents; and
each R² is independently selected from halo, C₁₋₆ alkyl, CN, C(O)R^{b2}, C(O)OR^{a2}, NR^{c2}C(O)R^{b2}, NR^{c2}C(O)OR^{a2}, and NR^{c2}S(O)₂R^{b2}, wherein the C₁₋₆ alkyl of R² are each optionally substituted with 1, 2, 3, or 4 independently selected R^{2A} substituents.

In some embodiments of any of Formulas I-VIIIo:
R¹ is C₁-3 alkyl:
Cy² is selected from cyclobutyl, cyclopentyl, deuterocyclopentyl, cyclohexyl, tetrahydrofuranyl, bicyclo[2.2.1]heptanyl, bicyclo[2.2.2]octanyl, spiro[3.3]heptanyl, 3-azabicyclo[3.1.0]hexanyl, 2-azaspiro[3.3]heptanyl, and 2-azabicyclo[2.2.1]heptanyl, wherein the cyclobutyl, cyclopentyl, deuterocyclopentyl, cyclohexyl, tetrahydrofuranyl, bicyclo[2.2.1]heptanyl, bicyclo[2.2.2]octanyl, spiro[3.3]heptanyl, 3-azabicyclo[3.1.0]hexanyl, 2-azaspiro[3.3]heptanyl, and 2-azabicyclo[2.2.1]heptanyl of Cy² are each optionally substituted by 1, 2, 3 or 4 independently selected R² substituents;
each R² is independently selected from halo, C₁₋₆ alkyl, CN, OR^{a2}, C(O)R^{b2}, C(O)NR²R^{d2}, C(O)OR^{a2}, NR^{c2}C(O)R^{b2}, NR^{c2}C(O)OR^{a2}, and NR^{c2}S(O)₂R^{b2}, wherein the C₁₋₆ alkyl of R² are each optionally substituted with 1, 2, 3, or 4 independently selected R^{2A} substituents;
each R^{a2}, R^{b2}, R^{c2}, and R^{d2} is independently selected from H, methyl, trideuteromethyl, ethyl, propynyl, cyclopropyl, cyclobutyl, phenyl, and tetrahydropyranyl, wherein the methyl, ethyl, propynyl, cyclopropyl, cyclobutyl, phenyl, and tetrahydropyranyl of R^{a2}, R^{b1}, R^{c2}, and R^{d2} are each optionally substituted with 1, 2, 3, or 4 independently selected R^{2A} substituents;
each R^{2A} is independently selected from halo and OR^{a21};
each R^{a21} is independently selected from H and C₁₋₆ alkyl; and
R³ is H.

In some embodiments of the previous embodiment:
Cy² is selected from cyclobutyl, cyclopentyl, deuterocyclopentyl, cyclohexyl, tetrahydrofuranyl, spiro[3.3]heptanyl, 3-azabicyclo[3.1.0]hexanyl, 2-azaspiro[3.3]heptanyl, and 2-azabicyclo[2.2.1]heptanyl, wherein the cyclobutyl, cyclopentyl, deuterocyclopentyl, cyclohexyl, tetrahydrofuranyl, spiro[3.3]heptanyl, 3-azabicyclo[3.1.0]hexanyl, 2-azaspiro[3.3]heptanyl, and 2-azabicyclo[2.2.1]heptanyl of Cy² are each optionally substituted by 1, 2, 3 or 4 independently selected R² substituents;
each R² is independently selected from halo, C₁₋₆ alkyl, CN, C(O)R^{b2}, C(O)OR^{a2}, NR^{c2}C(O)R^{b2}, NR^{c2}C(O)OR^{a2}, and NR^{c2}S(O)₂R^{b2}, wherein the C₁₋₆ alkyl of R² are each optionally substituted with 1, 2, 3, or 4 independently selected R^{2A} substituents; and
each R^{a2}, R^{b2}, R^{c2}, and R^{d2} is independently selected from H, methyl, ethyl, propynyl, cyclopropyl, cyclobutyl, phenyl, and tetrahydropyranyl, wherein the methyl, ethyl, propynyl, cyclopropyl, cyclobutyl, phenyl, and tetrahydropyranyl of R^{a2}, R^{b1}, R^{c2}, and R^{d2} are each optionally substituted with 1, 2, 3, or 4 independently selected R^{2A} substituents.

In some embodiments, the compound of provided herein is selected from:
ethyl 2-((3′-cyano-5-((1-((1R,3R)-3-((methoxycarbonyl)amino)cyclopentyl)-3-methyl-2-oxo-2,3-dihydro-1H-imidazo[4,5-c]pyridin-6-yl)amino)-[1, l′-bipheny]]-3-yl)amino)oxazole-5-carboxylate;
methyl ((1R,3R)-3-(6-((3′-cyano-5-(4-fluorotetrahydro-2H-pyran-4-carboxamido)-[1,1′-bipheny]]-3-yl)amino)-3-methyl-2-oxo-2,3-dihydro-1H-imidazo[4,5-c]pyridin-1-yl)cyclopentyl)carbamate;
methyl ((1R,3R)-3-(3-(methyl-d₃)-6-((4-(1-methyl-1,6-diazaspiro[3.3]heptane-6-carbonyl)-8-(trifluoromethyl)quinolin-2-yl)amino)-2-oxo-2,3-dihydro-1H-imidazo[4,5-c]pyridin-1-yl)cyclopentyl)carbamate;
methyl ((1R,3R)-3-(6-((6-(4-(hydroxymethyl)-3-methylphenyl)pyridin-2-yl)amino)-3-methyl-2-oxo-2,3-dihydro-1H-imidazo[4,5-c]pyridin-1-yl)cyclopentyl)carbamate;
methyl ((1R,3R)-3-(6-((6′-methoxy-[2,3′-bipyridin]-6-yl)amino)-3-methyl-2-oxo-2,3-dihydro-1H-imidazo[4,5-c]pyridin-1-yl)cyclopentyl)carbamate;
methyl ((1R,3R)-3-(6-((4-(4-cyanopiperidine-1-carbonyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(3-methyl-6-((6-(3-methyl-1H-indazol-5-yl)pyridin-2-yl)amino)-2-oxo-2,3-dihydro-1H-imidazo[4,5-c]pyridin-1-yl)cyclopentyl)carbamate;
methyl ((1R,3R)-3-(6-((6-(2,3-dihydrobenzo[b][1,4]dioxin-6-yl)pyridin-2-yl)amino)-3-methyl-2-oxo-2,3-dihydro-1H-imidazo[4,5-c]pyridin-1-yl)cyclopentyl)carbamate;
methyl ((1R,3R)-3-(3-methyl-2-oxo-6-((6-(quinolin-6-yl)pyridin-2-yl)amino)-2,3-dihydro-1H-imidazo[4,5-c]pyridin-1-yl)cyclopentyl)carbamate;
methyl ((1R,3R)-3-(6-((4-(1-hydroxy-3-azabicyclo[3.1.0]hexane-3-carbonyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(3-methyl-6-((6-(4-methyl-3,4-dihydro-2H-benzo[b][1,4]oxazin-7-yl)pyridin-2-yl)amino)-2-oxo-2,3-dihydro-1H-imidazo[4,5-c]pyridin-1-yl)cyclopentyl)carbamate;
methyl ((1R,3R)-3-(6-((4-(2-oxa-5-azabicyclo[2.2.1]heptane-5-carbonyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((4-(2-oxa-5-azabicyclo[4.1.0]heptane-5-carbonyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl (3-((1-((1R,3R)-3-((methoxycarbonyl)amino)cyclopentyl)-3-methyl-2-oxo-2,3-dihydro-1H-imidazo[4,5-c]pyridin-6-yl)amino)-5-(1-methyl-1H-1,2,3-triazol-4-yl)phenyl)carbamate;
methyl (3-((1-((1R,3R)-3-((methoxycarbonyl)amino)cyclopentyl)-3-methyl-2-oxo-2,3-dihydro-1H-imidazo[4,5-c]pyridin-6-yl)amino)-5-(1-methylpiperidin-4-yl)phenyl)carbamate;
methyl ((1R,3R)-3-(6-((4-(3,3-difluoropiperidine-1-carbonyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((3-((R)-2,2-difluorocyclopropane-1-carboxamido)-5-(tetrahydro-2H-pyran-4-yl)phenyl)amino)-3-methyl-2-oxo-2,3-dihydro-1H-imidazo[4,5-c]pyridin-1-yl)cyclopentyl)carbamate;
methyl ((1R,3R)-3-(6-((6-((3,3-difluorocyclopentyl)amino)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((3-(cyclobutanecarboxamido)-5-(tetrahydro-2H-pyran-4-yl)phenyl)amino)-3-methyl-2-oxo-2,3-dihydro-1H-imidazo[4,5-c]pyridin-1-yl)cyclopentyl)carbamate;
methyl ((1R,3R)-3-(3-(methyl-d₃)-6-((6-(1-methyl-1H-pyrazol-4-yl)-8-(trifluoromethyl)quinolin-2-yl)amino)-2-oxo-2,3-dihydro-1H-imidazo[4,5-c]pyridin-1-yl)cyclopentyl)carbamate;
methyl (3-((1-((1R,3R)-3-((methoxycarbonyl)amino)cyclopentyl)-3-methyl-2-oxo-2,3-dihydro-1H-imidazo[4,5-c]pyridin-6-yl)amino)-5-(tetrahydro-2H-pyran-3-yl)phenyl)carbamate;
methyl ((1R,3R)-3-(6-((6-((4-cyanocyclohexyl)oxy)pyridin-2-yl)amino)-3-methyl-2-oxo-2,3-dihydro-1H-imidazo[4,5-c]pyridin-1-yl)cyclopentyl)carbamate;
methyl ((1R,3R)-3-(3-methyl-2-oxo-6-((6-(quinolin-6-yloxy)pyridin-2-yl)amino)-2,3-dihydro-1H-imidazo[4,5-c]pyridin-1-yl)cyclopentyl)carbamate;
methyl (3-((1-((1r,3r)-3-(cyclopropanecarboxamido)cyclobutyl)-3-methyl-2-oxo-2,3-dihydro-1H-imidazo[4,5-c]pyridin-6-yl)amino)-5-(1-methyl-1H-pyrazol-4-yl)phenyl)carbamate;
methyl (3-((1-(6-((ethoxycarbonyl)amino)spiro[3.3]heptan-2-yl)-3-methyl-2-oxo-2,3-dihydro-1H-imidazo[4,5-c]pyridin-6-yl)amino)-5-(1-methyl-1H-pyrazol-4-yl)phenyl)carbamate;
methyl (3-((1-(6-(cyclopropanesulfonamido)spiro[3.3]heptan-2-yl)-3-methyl-2-oxo-2,3-dihydro-1H-imidazo[4,5-c]pyridin-6-yl)amino)-5-(1-methyl-1H-pyrazol-4-yl)phenyl)carbamate;
methyl 6-(6-((3-((methoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)-2-azaspiro[3.3]heptane-2-carboxylate:
methyl (3-((1-((1r,3r)-3-cyanocyclobutyl)-3-methyl-2-oxo-2,3-dihydro-1H-imidazo[4,5-c]pyridin-6-yl)amino)-5-(1-methyl-1H-pyrazol-4-yl)phenyl)carbamate;
methyl (3-((1-((1r,3r)-3-((methoxycarbonyl)amino)-3-methylcyclobutyl)-3-methyl-2-oxo-2,3-dihydro-1H-imidazo[4,5-c]pyridin-6-yl)amino)-5-(1-methyl-1H-pyrazol-4-yl)phenyl)carbamate;
methyl (R)-(3-((1-(3,3-difluorocyclopentyl)-3-methyl-2-oxo-2,3-dihydro-1H-imidazo[4,5-c]pyridin-6-yl)amino)-5-(1-methyl-1H-pyrazol-4-yl)phenyl)carbamate;
methyl (3-((1-((1R,3S)-3-fluorocyclopentyl)-3-methyl-2-oxo-2,3-dihydro-1H-imidazo[4,5-c]pyridin-6-yl)amino)-5-(1-methyl-1H-pyrazol-4-yl)phenyl)carbamate;
methyl ((1R,3R)-3-(3-methyl-6-((6-(1-methyl-1H-pyrazol-4-yl)quinolin-2-yl)amino)-2-oxo-2,3-dihydro-1H-imidazo[4,5-c]pyridin-1-yl)cyclopentyl)carbamate;
methyl ((1R,3R)-3-(3-methyl-6-((8-(1-methyl-1H-pyrazol-4-yl)quinolin-2-yl)amino)-2-oxo-2,3-dihydro-1H-imidazo[4,5-c]pyridin-1-yl)cyclopentyl)carbamate;
methyl ((1R,3R)-3-(3-methyl-6-((3-(1-methyl-1H-pyrazol-4-yl)imidazo[1,2-b]pyridazin-6-yl)amino)-2-oxo-2,3-dihydro-1H-imidazo[4,5-c]pyridin-1-yl)cyclopentyl)carbamate;
methyl ((1R,3R)-3-(3-methyl-6-((2-(1-methyl-1H-pyrazol-4-yl)pyrazolo[1,5-a]pyrimidin-5-yl)amino)-2-oxo-2,3-dihydro-1H-imidazo[4,5-c]pyridin-1-yl)cyclopentyl)carbamate;
methyl ((1R,3R)-3-(3-methyl-6-((3-(1-methyl-1H-pyrazol-4-yl)pyrazolo[1,5-a]pyrimidin-5-yl)amino)-2-oxo-2,3-dihydro-1H-imidazo[4,5-c]pyridin-1-yl)cyclopentyl)carbamate;
methyl ((1R,3R)-3-(3-methyl-2-oxo-6-((6-((tetrahydro-2H-pyran-4-yl)oxy)pyridin-2-yl)amino)-2,3-dihydro-1H-imidazo[4,5-c]pyridin-1-yl)cyclopentyl)carbamate; methyl ((1R,3R)-3-(3-methyl-6-((3-(1-methyl-1H-imidazol-5-yl)imidazo[1,2-b]pyridazin-6-yl)amino)-2-oxo-2,3-dihydro-1H-imidazo[4,5-c]pyridin-1-yl)cyclopentyl)carbamate;
methyl (1R,5S)-6-(6-((3-((methoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)-3-azabicyclo[3.1.0]hexane-3-carboxylate;
methyl (3-((1-(2-acetyl-2-azabicyclo[2.2.1]heptan-5-yl)-3-methyl-2-oxo-2,3-dihydro-1H-imidazo[4,5-c]pyridin-6-yl)amino)-5-(1-methyl-1H-pyrazol-4-yl)phenyl)carbamate;
methyl ((1R,3R)-3-(6-((3-((3,3-difluorocyclobutane)-1-sulfonamido)-5-(tetrahydro-2H-pyran-4-yl)phenyl)amino)-3-methyl-2-oxo-2,3-dihydro-1H-imidazo[4,5-c]pyridin-1-yl)cyclopentyl)carbamate;
methyl (3-((1-(4-((methoxycarbonyl)amino)-4-methylcyclohexyl)-3-methyl-2-oxo-2,3-dihydro-1H-imidazo[4,5-c]pyridin-6-yl)amino)-5-(1-methyl-1H-pyrazol-4-yl)phenyl)carbamate;
2-methoxyethyl ((1R,3R)-3-(6-((3-((cyclobutoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate;
methyl ((1R,3R)-3-(6-((3-((cyclobutoxycarbonyl)amino)-5-(tetrahydro-2H-pyran-4-yl)phenyl)amino)-3-methyl-2-oxo-2,3-dihydro-1H-imidazo[4,5-c]pyridin-1-yl)cyclopentyl)carbamate;
cyclobutyl ((1R,3R)-3-(6-((3-((cyclobutoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate;
ethyl ((1R,3R)-3-(6-((3-((cyclobutoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate;
prop-2-yn-1-yl ((1R,3R)-3-(6-((3-((cyclobutoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate;
tetrahydro-2H-pyran-4-yl ((1R,3R)-3-(6-((3-((cyclobutoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate;
4-fluorophenyl ((1R,3R)-3-(6-((3-((cyclobutoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate;
methyl ((1R,3R)-3-(6-((6-cyclopropylpyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((6-(3-cyanophenyl)-4-(1-methyl-1H-pyrazol-4-yl)pyridin-2-yl)amino)-3-methyl-2-oxo-2,3-dihydro-1H-imidazo[4,5-c]pyridin-1-yl)cyclopentyl)carbamate;
methyl ((1R,3R)-3-(3-(methyl-d₃)-6-((4-(morpholine-4-carbonyl)-8-(trifluoromethyl)quinolin-2-yl)amino)-2-oxo-2,3-dihydro-1H-imidazo[4,5-c]pyridin-1-yl)cyclopentyl)carbamate;
methyl ((1R,3R)-3-(6-((3-((methoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate;
methyl ((1R,3R)-3-(6-((3-cyclopropylimidazo[1,2-b]pyridazin-6-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((3-((cyclopropoxycarbonyl)amino)-5-methoxyphenyl)amino)-3-methyl-2-oxo-2,3-dihydro-1H-imidazo[4,5-c]pyridin-1-yl)cyclopentyl)carbamate;
methyl ((1R,3R)-3-(6-((3-((cyclobutoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate;
methyl ((1R,3R)-3-(6-((3-(cyclopropanecarboxamido)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate;
methyl ((1R,3R)-3-(3-(methyl-d₃)-6-((8-(1-methylcyclopropyl)quinolin-2-yl)amino)-2-oxo-2,3-dihydro-1H-imidazo[4,5-c]pyridin-1-yl)cyclopentyl)carbamate;
methyl ((1R,3R)-3-(6-((3-(3-fluoro-1-(methyl-d₃)-1H-pyrazol-4-yl)imidazo[1,2-b]pyridazin-6-yl)amino)-3-methyl-2-oxo-2,3-dihydro-1H-imidazo[4,5-c]pyridin-1-yl)cyclopentyl)carbamate;
methyl ((1R,3R)-3-(6-((4-(cyclobutoxymethyl)-6-(2,3-dihydrobenzo[b][1,4]dioxin-6-yl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((6-(3-aminopyrrolidin-1-yl)pyridin-2-yl)amino)-3-methyl-2-oxo-2,3-dihydro-1H-imidazo[4,5-c]pyridin-1-yl)cyclopentyl)carbamate;
methyl ((1R,3R)-3-(6-((4-((cyclopentylamino)methyl)-6-(2,3-dihydrobenzo[b][1,4]dioxin-6-yl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(3-methyl-2-oxo-6-((2-(piperidin-1-yl)pyrimidin-4-yl)amino)-2,3-dihydro-1H-imidazo[4,5-c]pyridin-1-yl)cyclopentyl)carbamate;
methyl ((1R,3R)-3-(6-((2′-methoxy-4-methyl-[2,4′-bipyridin]-6-yl)amino)-3-methyl-2-oxo-2,3-dihydro-1H-imidazo[4,5-c]pyridin-1-yl)cyclopentyl)carbamate;
methyl ((1R,3R)-3-(3-methyl-2-oxo-6-((4-(pyridin-4-yl)thiazol-2-yl)amino)-2,3-dihydro-1H-imidazo[4,5-c]pyridin-1-yl)cyclopentyl)carbamate;
methyl ((1R,3R)-3-(6-((3-fluoro-6′-(trifluoromethyl)-[2,3′-bipyridin]-6-yl)amino)-3-methyl-2-oxo-2,3-dihydro-1H-imidazo[4,5-c]pyridin-1-yl)cyclopentyl)carbamate;
methyl ((1R,3R)-3-(6-((6-(2,3-dihydrobenzo[b][1,4]dioxin-6-yl)-4-(((S)-3-fluoropyrrolidin-1-yl)methyl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((6-(4-(cyanomethyl)phenyl)-5-fluoropyridin-2-yl)amino)-3-methyl-2-oxo-2,3-dihydro-1H-imidazo[4,5-c]pyridin-1-yl)cyclopentyl)carbamate;
methyl ((1R,3R)-3-(6-((6-(4-cyclopropylphenyl)-5-fluoropyridin-2-yl)amino)-3-methyl-2-oxo-2,3-dihydro-1H-imidazo[4,5-c]pyridin-1-yl)cyclopentyl)carbamate;
methyl ((1R,3R)-3-(6-((5-fluoro-6-(4-(trifluoromethoxy)phenyl)pyridin-2-yl)amino)-3-methyl-2-oxo-2,3-dihydro-1H-imidazo[4,5-c]pyridin-1-yl)cyclopentyl)carbamate;
methyl ((1R,3R)-3-(6-((6-(2,3-dihydrobenzo[b][1,4]dioxin-6-yl)-4-(morpholinomethyl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((2-(2-methoxypyridin-4-yl)pyrimidin-4-yl)amino)-3-(methyl-d3)-2-oxo-2,3-dihydro-1H-imidazo[4,5-c]pyridin-1-yl)cyclopentyl)carbamate;
methyl ((1R,3R)-3-(6-((6-(2,3-dihydrobenzo[b][1,4]dioxin-6-yl)-4-(octahydro-2H-pyrido[1,2-a]pyrazine-2-carbonyl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((2-(5-azaspiro[2.4]heptan-5-yl)pyrimidin-4-yl)amino)-3-methyl-2-oxo-2,3-dihydro-1H-imidazo[4,5-c]pyridin-1-yl)cyclopentyl)carbamate;
methyl ((1R,3R)-3-(6-((2-(3,3-difluoropyrrolidin-1-yl)pyrimidin-4-yl)amino)-3-methyl-2-oxo-2,3-dihydro-1H-imidazo[4,5-c]pyridin-1-yl)cyclopentyl)carbamate;
methyl ((1R,3R)-3-(3-methyl-2-oxo-6-((2-(4-(trifluoromethyl)piperidin-1-yl)pyrimidin-4-yl)amino)-2,3-dihydro-1H-imidazo[4,5-c]pyridin-1-yl)cyclopentyl)carbamate;
methyl ((1R,3R)-3-(6-((6-(2,3-dihydrobenzo[b][1,4]dioxin-6-yl)-4-((R)-3-fluoropyrrolidine-1-carbonyl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(3-methyl-2-oxo-6-((1-(pyridin-4-yl)-1H-pyrazol-3-yl)amino)-2,3-dihydro-1H-imidazo[4,5-c]pyridin-1-yl)cyclopentyl)carbamate;
methyl ((1R,3R)-3-(6-((6-(2,3-dihydrobenzo[b][1,4]dioxin-6-yl)-4-(morpholine-4-carbonyl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((2-((3,3-difluorocyclopentyl)amino)pyrimidin-4-yl)amino)-3-methyl-2-oxo-2,3-dihydro-1H-imidazo[4,5-c]pyridin-1-yl)cyclopentyl)carbamate;
methyl ((1R,3R)-3-(6-((2-(3,3-difluoropyrrolidin-1-yl)-6-methylpyrimidin-4-yl)amino)-3-methyl-2-oxo-2,3-dihydro-1H-imidazo[4,5-c]pyridin-1-yl)cyclopentyl)carbamate;
methyl ((1R,3R)-3-(3-methyl-2-oxo-6-((2-((tetrahydro-2H-pyran-4-yl)amino)pyrimidin-4-yl)amino)-2,3-dihydro-1H-imidazo[4,5-c]pyridin-1-yl)cyclopentyl)carbamate;
methyl ((1R,3R)-3-(3-methyl-2-oxo-6-((6-(piperidine-1-carbonyl)pyridin-2-yl)amino)-2,3-dihydro-1H-imidazo[4,5-c]pyridin-1-yl)cyclopentyl)carbamate; and
methyl ((1R,3R)-3-(6-((6-(cyclohexylcarbamoyl)pyridin-2-yl)amino)-3-methyl-2-oxo-2,3-dihydro-1H-imidazo[4,5-c]pyridin-1-yl)cyclopentyl)carbamate;
or a pharmaceutically acceptable salt thereof.

In some embodiments, the present application provides a compound selected from:
cyclobutyl (3-((1-cyclopentyl-3-methyl-2-oxo-2,3-dihydro-1H-imidazo[4,5-c]pyridin-6-yl)amino)-5-(1-methyl-1H-pyrazol-4-yl)phenyl)carbamate;
cyclobutyl (3-((1-cyclopentyl-3-methyl-2-oxo-2,3-dihydro-1H-imidazo[4,5-c]pyridin-6-yl)amino)-5-(1-cyclopropyl-1H-pyrazol-4-yl)phenyl)carbamate;
cyclobutyl (3-(1-cyclopropyl-1H-pyrazol-4-yl)-5-((3-methyl-2-oxo-1-(tetrahydrofuran-3-yl)-2,3-dihydro-1H-imidazo[4,5-c]pyridin-6-yl)amino)phenyl)carbamate;
methyl ((1R,3R)-3-(6-((1-(2-methoxyethyl)-3-(1-(trifluoromethyl)-1H-pyrazol-4-yl)-1H-pyrrolo[3,2-b]pyridin-5-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((3-(1-(difluoromethyl)-1H-pyrazol-4-yl)-1-methyl-2-oxo-7-(4-(trifluoromethyl)pyridin-3-yl)-2,3-dihydro-1H-imidazo[4,5-b]pyridin-5-yl)amino)-3-(methyl-d₃)-2-oxo-2,3-dihydro-1H-imidazo[4,5-c]pyridin-1-yl)cyclopentyl)carbamate;
N-(6-(3-(methyl-d₃)-6-((4-(morpholine-4-carbonyl)-8-(trifluoromethyl)quinolin-2-yl)amino)-2-oxo-2,3-dihydro-1H-imidazo[4,5-c]pyridin-1-yl)spiro[3.3]heptan-2-yl)cyclopropanecarboxamide;
N-((1s,4s)-1-methyl-4-(3-(methyl-d₃)-6-((2-methyl-3-(1-(trifluoromethyl)-1H-pyrazol-4-yl)-2H-pyrazolo[4,3-b]pyridin-5-yl)amino)-2-oxo-2,3-dihydro-1H-imidazo[4,5-c]pyridin-1-yl)cyclohexyl)cyclopropanecarboxamide;
N-((1S,3R)-3-(6-((6-(5-chlorothiazol-2-yl)-4-(2-hydroxypropan-2-yl)pyridin-2-yl)amino)-3-(methyl-d₃)-2-oxo-2,3-dihydro-1H-imidazo[4,5-c]pyridin-1-yl)-1-methylcyclopentyl)cyclopropanecarboxamide;
methyl ((1R,3R)-3-(6-((6-(5-chlorothiazol-2-yl)-4-(2-hydroxypropan-2-yl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((4-(2-hydroxypropan-2-yl)-6-(1-methyl-1H-pyrazol-4-yl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((4-(2-hydroxypropan-2-yl)-6-(2-(trifluoromethyl)thiazol-5-yl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((8-(2-cyanopropan-2-yl)-4-(morpholine-4-carbonyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((3-(1-(difluoromethyl)-1H-pyrazol-4-yl)-2-methyl-8-(4-(trifluoromethyl)pyridin-3-yl)imidazo[1,2-b]pyridazin-6-yl)amino)-3-(methyl-d₃)-2-oxo-2,3-dihydro-1H-imidazo[4,5-c]pyridin-1-yl)cyclopentyl-3-d)carbamate;
methyl ((1R,3R)-3-(6-((5-methoxy-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((6-(5-(2-hydroxypropan-2-yl)thiazol-2-yl)-4-methylpyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((4-((3-(difluoromethyl)azetidin-1-yl)methyl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((6-cyclobutoxy-5-(1-methyl-1H-pyrazol-4-yl)-4-(morpholine-4-carbonyl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((2-(5-(difluoromethyl)thiophen-2-yl)-6-(hydroxymethyl)pyrimidin-4-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((6-(5-(difluoromethyl)thiophen-2-yl)-4-(morpholine-4-carbonyl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((4-(1-acetylpiperidin-4-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl)carbamate;
6-((3-(1-(difluoromethyl)-1H-pyrazol-4-yl)-2-methyl-8-(4-(trifluoromethyl)pyridin-3-yl)imidazo[1,2-b]pyridazin-6-yl)amino)-1-((1r,3r)-3-(2-hydroxypropan-2-yl)cyclobutyl)-3-(methyl-d₃)-1,3-dihydro-2H-imidazo[4,5-c]pyridin-2-one:
methyl ((1R,3R)-3-(6-((6-(5-(2-hydroxypropan-2-yl)thiazol-2-yl)-4-(1-methyl-1H-pyrazol-4-yl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(3-(methyl-d₃)-2-oxo-6-((6-(4-(trifluoromethyl)-1H-pyrazol-1-yl)pyridin-2-yl)amino)-2,3-dihydro-1H-imidazo[4,5-c]pyridin-1-yl)cyclopentyl)carbamate;
methyl ((1R,3R)-3-(3-(methyl-d₃)-2-oxo-6-((1-(tetrahydro-2H-pyran-4-yl)-3-(1-(trifluoromethyl)-1H-pyrazol-4-yl)-1H-pyrrolo[3,2-b]pyridin-5-yl)amino)-2,3-dihydro-1H-imidazo[4,5-c]pyridin-1-yl)cyclopentyl)carbamate;
methyl ((1R,3R)-3-(6-((1-(2-hydroxy-2-methylpropyl)-3-(1-(trifluoromethyl)-1H-pyrazol-4-yl)-1H-pyrrolo[3,2-b]pyridin-5-yl)amino)-3-(methyl-d₃)-2-oxo-2,3-dihydro-1H-imidazo[4,5-c]pyridin-1-yl)cyclopentyl)carbamate;
methyl ((3R)-3-(3-(methyl-d₃)-2-oxo-6-((1-(tetrahydro-2H-pyran-4-yl)-3-(1-(trifluoromethyl)-1H-pyrazol-4-yl)-1H-pyrrolo[3,2-b]pyridin-5-yl)amino)-2,3-dihydro-1H-imidazo[4,5-c]pyridin-1-yl)cyclopentyl-1-d)carbamate;
methyl ((3R)-3-(3-(methyl-d₃)-2-oxo-6-((1-(tetrahydro-2H-pyran-4-yl-4-d)-3-(1-(trifluoromethyl)-1H-pyrazol-4-yl)-1H-pyrrolo[3,2-b]pyridin-5-yl)amino)-2,3-dihydro-1H-imidazo[4,5-c]pyridin-1-yl)cyclopentyl-1-d)carbamate;
methyl ((3R)-3-(6-((1-(1-cyanocyclopropyl)-3-(1-(trifluoromethyl)-1H-pyrazol-4-yl)-1H-pyrrolo[3,2-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1-cyanocyclopropyl)methyl)-3-(1-(difluoromethyl)-1H-pyrazol-4-yl)-2-oxo-2,3-dihydro-1H-imidazo[4,5-b]pyridin-5-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((3-(1-(difluoromethyl)-1H-pyrazol-4-yl)-7-(2-hydroxypropan-2-yl)-2-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl)carbamate;
N-((1R,3R)-1-methyl-3-(3-(methyl-d₃)-6-((4-(morpholine-4-carbonyl)-8-(trifluoromethyl)quinolin-2-yl)amino)-2-oxo-2,3-dihydro-1H-imidazo[4,5-c]pyridin-1-yl)cyclopentyl)cyclopropanecarboxamide;
N-((1S,3R)-3-(6-((7-(hydroxymethyl)-2-methyl-3-(1-(trifluoromethyl)-1H-pyrazol-4-yl)-2H-pyrazolo[4,3-b]pyridin-5-yl)amino)-3-(methyl-d₃)-2-oxo-2,3-dihydro-1H-imidazo[4,5-c]pyridin-1-yl)-1-methylcyclopentyl)cyclopropanecarboxamide;
methyl ((1R,3R)-3-(6-((1-(2-hydroxy-2-methylpropyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)cyclopentyl)carbamate;
N-((1S,3R)-3-(6-((1-(2-hydroxy-2-methylpropyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)-1-methylcyclopentyl)cyclopropanecarboxamide;
N-((1s,4s)-4-(6-((1-(2-hydroxy-2-methylpropyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)-1-methylcyclohexyl)cyclopropanecarboxamide;
N-((1S,3R)-3-(6-((2-((1s,3s)-3-hydroxycyclobutyl)-3-(1-(trifluoromethyl)-1H-pyrazol-4-yl)-2H-pyrazolo[4,3-b]pyridin-5-yl)amino)-3-(methyl-d₃)-2-oxo-2,3-dihydro-1H-imidazo[4,5-c]pyridin-1-yl)-1-methylcyclopentyl)cyclopropanecarboxamide;
methyl ((1R,3R)-3-(6-((2-(2-hydroxy-2-methylpropyl)-3-(1-(trifluoromethyl)-1H-pyrazol-4-yl)-2H-pyrazolo[4,3-b]pyridin-5-yl)amino)-3-(methyl-d₃)-2-oxo-2,3-dihydro-1H-imidazo[4,5-c]pyridin-1-yl)cyclopentyl)carbamate;
N-((1S,3R)-3-(6-((2-(2-hydroxy-2-methylpropyl)-3-(1-(trifluoromethyl)-1H-pyrazol-4-yl)-2H-pyrazolo[4,3-b]pyridin-5-yl)amino)-3-(methyl-d₃)-2-oxo-2,3-dihydro-1H-imidazo[4,5-c]pyridin-1-yl)-1-methylcyclopentyl)cyclopropanecarboxamide;
methyl ((3R)-3-(6-((1-(2-hydroxy-2-methylpropyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((3R)-3-(6-((1-(2-hydroxy-2-methylpropyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)cyclopentyl-1-d)carbamate;
N-((1s,4s)-4-(6-((1-((1r,4r)-4-hydroxycyclohexyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)-1-methylcyclohexyl)cyclopropanecarboxamide;
N-((1s,4s)-1-methyl-4-(3-(methyl-d₃)-6-((2-methyl-7-(morpholine-4-carbonyl)-3-(1-(trifluoromethyl)-1H-pyrazol-4-yl)-2H-pyrazolo[4,3-b]pyridin-5-yl)amino)-2-oxo-2,3-dihydro-1H-imidazo[4,5-c]pyridin-1-yl)cyclohexyl)cyclopropanecarboxamide;
methyl ((1S,3R)-3-(6-((1-(2-hydroxy-2-methylpropyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)-1-methylcyclopentyl)carbamate;
N-((1S,3R)-3-(6-((1-(2-hydroxy-2-methylpropyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)-1-methylcyclopentyl)acetamide:
methyl ((3R)-3-(6-((7-(2-hydroxypropan-2-yl)-3-(1-(trifluoromethyl)-1H-pyrazol-4-yl)-1H-pyrrolo[3,2-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((3R)-3-(6-((4-(2-hydroxypropan-2-yl)-1-(1-(trifluoromethyl)-1H-pyrazol-4-yl)-1H-pyrrolo[2,3-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((3R)-3-(3-(methyl-d₃)-2-oxo-6-((1-(tetrahydro-2H-pyran-4-yl)-3-(1-(trifluoromethyl)-1H-pyrazol-4-yl)-1H-pyrazolo[4,3-b]pyridin-5-yl)amino)-2,3-dihydro-1H-imidazo[4,5-c]pyridin-1-yl)cyclopentyl-1-d)carbamate;
methyl ((3R)-3-(6-((1-((1-cyanocyclopropyl)methyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((3R)-3-(6-((2-((1-cyanocyclopropyl)methyl)-3-(1-(trifluoromethyl)-1H-pyrazol-4-yl)-2H-pyrazolo[4,3-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(3-(methyl-d₃)-6-((6-methyl-4-(morpholine-4-carbonyl)-8-(trifluoromethyl)quinolin-2-yl)amino)-2-oxo-2,3-dihydro-1H-imidazo[4,5-c]pyridin-1-yl)cyclopentyl)carbamate;
N-((1S,3R)-3-(6-((6-(5-chlorothiazol-2-yl)-4-(2-hydroxypropan-2-yl)pyridin-2-yl)amino)-3-(methyl-d₃)-2-oxo-2,3-dihydro-1H-imidazo[4,5-c]pyridin-1-yl)-1-methylcyclopentyl)acetamide;
4-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)-N-(methyl-d₃)bicyclo[2.2.2]octane-1-carboxamide;
N-cyclopropyl-4-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)bicyclo[2.2.2]octane-1-carboxamide;
4-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)bicyclo[2.2.2]octane-1-carboxamide;
N-(4-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)bicyclo[2.2.2]octan-1-yl)acetamide;
4-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)bicyclo[2.2.1]heptane-1-carboxamide;
N-(4-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)bicyclo[2.2.1]heptan-1-yl)acetamide;
N-((1S,3R)-3-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)-1-methylcyclopentyl)propionamide;
methyl ((1R,3R)-3-(6-((4-(2-hydroxypropan-2-yl)-6-(5-(2-hydroxypropan-2-yl)thiazol-2-yl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((6-(5-(1,1,1,3,3,3-hexafluoro-2-hydroxypropan-2-yl)thiazol-2-yl)-4-(2-hydroxypropan-2-yl)pyridin-2-yl)amino)-3-(methyl-d₃)-2-oxo-2,3-dihydro-1H-imidazo[4,5-c]pyridin-1-yl)cyclopentyl)carbamate;
N-((1S,3R)-3-(6-((4-(2-hydroxypropan-2-yl)-6-(2-(trifluoromethyl)thiazol-5-yl)pyridin-2-yl)amino)-3-(methyl-d₃)-2-oxo-2,3-dihydro-1H-imidazo[4,5-c]pyridin-1-yl)-1-methylcyclopentyl)acetamide;
methyl ((1R,3R)-3-(6-((6-(5-(1,1,1,3,3,3-hexafluoro-2-hydroxypropan-2-yl)thiazol-2-yl)-4-(2-hydroxypropan-2-yl)pyridin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((6-(5-(2-hydroxypropan-2-yl)thiazol-2-yl)-4-methylpyridin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((5-fluoro-6-(5-(2-hydroxypropan-2-yl)thiazol-2-yl)-4-methylpyridin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((6-(5-(2-hydroxypropan-2-yl)thiazol-2-yl)-4-(methyl-d₃)pyridin-2-yl)amino)-3-(methyl-d₃)-2-oxo-2,3-dihydro-1H-imidazo[4,5-c]pyridin-1-yl)cyclopentyl-1-d)carbamate;
N-((1S,3R)-3-(6-((4-(2-hydroxypropan-2-yl)-6-(2-isopropoxythiazol-5-yl)pyridin-2-yl)amino)-3-(methyl-d₃)-2-oxo-2,3-dihydro-1H-imidazo[4,5-c]pyridin-1-yl)-1-methylcyclopentyl)acetamide;
N-((1S,3R)-3-(6-((4-(2-hydroxypropan-2-yl)-6-(2-methoxythiazol-5-yl)pyridin-2-yl)amino)-3-(methyl-d₃)-2-oxo-2,3-dihydro-1H-imidazo[4,5-c]pyridin-1-yl)-1-methylcyclopentyl)acetamide;
N-((1S,3R)-3-(6-((6-(2-ethoxythiazol-5-yl)-4-(2-hydroxypropan-2-yl)pyridin-2-yl)amino)-3-(methyl-d₃)-2-oxo-2,3-dihydro-1H-imidazo[4,5-c]pyridin-1-yl)-1-methylcyclopentyl)acetamide;
methyl ((1R,3R)-3-(6-((6′-(5-(difluoromethyl)thiophen-2-yl)-4-(trifluoromethyl)-[3,4′-bipyridin]-2′-yl)amino)-3-(methyl-d₃)-2-oxo-2,3-dihydro-1H-imidazo[4,5-c]pyridin-1-yl)cyclopentyl)carbamate;
methyl ((1S,2S,4S)-2-hydroxy-4-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl-3-d)carbamate;
methyl ((1R,3R)-3-(3-(methyl-d₃)-6-((4-(1-methylpiperidin-4-yl)-6-(5-(trifluoromethyl)thiazol-2-yl)pyridin-2-yl)amino)-2-oxo-2,3-dihydro-1H-imidazo[4,5-c]pyridin-1-yl)cyclopentyl)carbamate;
methyl ((1R,3R)-3-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl-1-d)carbamate;
methyl ((1S,3R)-3-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl-1-d)carbamate;
methyl ((1S,3R)-3-(6-((3-(1-(difluoromethyl)-1H-pyrazol-4-yl)-2-methyl-8-(4-(trifluoromethyl)pyridin-3-yl)imidazo[1,2-b]pyridazin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((6-(5-(2-hydroxypropan-2-yl)thiazol-2-yl)-4-(tetrahydro-2H-pyran-4-yl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((6-(5-(2-hydroxypropan-2-yl)thiazol-2-yl)-4-(tetrahydro-2H-pyran-4-yl)pyridin-2-yl)amino)-3-(methyl-d₃)-2-oxo-2,3-dihydro-1H-imidazo[4,5-c]pyridin-1-yl)cyclopentyl-1-d)carbamate;
methyl ((1S,3R)-3-(6-((6-(5-(2-hydroxypropan-2-yl)thiazol-2-yl)-4-(tetrahydro-2H-pyran-4-yl)pyridin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((6′-(5-(2-hydroxypropan-2-yl)thiazol-2-yl)-4-(trifluoromethyl)-[3,4′-bipyridin]-2′-yl)amino)-3-(methyl-d₃)-2-oxo-2,3-dihydro-1H-imidazo[4,5-c]pyridin-1-yl)cyclopentyl-1-d)carbamate;
methyl ((1S,3R)-3-(6-((6′-(5-(2-hydroxypropan-2-yl)thiazol-2-yl)-4-(trifluoromethyl)-[3,4′-bipyridin]-2′-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((6-(5-(2-hydroxypropan-2-yl)thiazol-2-yl)-4-(1-methylpiperidin-4-yl)pyridin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((6′-(5-(2-hydroxypropan-2-yl)thiazol-2-yl)-2-(trifluoromethyl)-[3,4′-bipyridin]-2′-yl)amino)-3-(methyl-d₃)-2-oxo-2,3-dihydro-1H-imidazo[4,5-c]pyridin-1-yl)cyclopentyl-1-d)carbamate;
N-((1S,3R)-3-(6-((3-(1-(difluoromethyl)-1H-pyrazol-4-yl)-2-methyl-8-(4-(trifluoromethyl)pyridin-3-yl)imidazo[1,2-b]pyridazin-6-yl)amino)-3-(methyl-d₃)-2-oxo-2,3-dihydro-1H-imidazo[4,5-c]pyridin-1-yl)-1-methylcyclopentyl)acetamide; and
methyl ((1R,3R)-3-(6-((3-(1-(difluoromethyl)-1H-pyrazol-4-yl)-7-(2-hydroxypropan-2-yl)-2-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate

or a pharmaceutically acceptable salt thereof.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, divalent linking substituents are described. It is specifically intended that each divalent linking substituent include both the forward and backward forms of the linking substituent. For example, —NR(CR′R″)ₙ— includes both —NR(CR′R″)ₙ— and —(CR′R″)ₙNR—. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from.”

Throughout the definitions, the term “Cₙ₋ₘ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms. The term “Cₙ₋ₘ alkyl” is understood to include deuterated analogs of alkyl groups as defined herein, including but not limited to, groups such as trideuteromethyl (CD₃), pentadeuteroethyl (CD₂CD₃), and the like.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms. The term “Cₙ₋ₘ alkenyl” is understood to include deuterated analogs of alkenyl groups as defined herein, including but not limited to, groups such as trideuteroethenyl (—CD═CD₂), tetradeuteropropenyl, (-CD═CD-CD₂), and the like.

As used herein, “Cₙ₋ₘ alkynyl” refers to an alkyl group having one or more triple carbon-carbon bonds and having n to m carbons. Example alkynyl groups include, but are not limited to, ethynyl, propynyl (e.g., propyn-1-yl, propyn-2-yl, prop-2-yn-1-yl), and the like. In some embodiments, the alkynyl moiety contains 2 to 6, 2 to 4, or 2 to 3 carbon atoms. The term “Cₙ₋ₘ alkynyl” is understood to include deuterated analogs of alkynyl groups as defined herein, including but not limited to, groups such as deuteroethynyl (—C≡CD), trideuteropropyn-1-yl, (—C≡CCD₃), and the like.

As used herein, the term “Cₙ₋ₘ alkoxy”, employed alone or in combination with other terms, refers to a group of formula-O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms. The term “Cₙ₋ₘ alkoxy” is understood to include deuterated analogs of the alkyl moiety of the alkoxy group as defined herein, including but not limited to, groups such as trideuteromethoxy (—OCD₃), pentadeuteroethoxy (—OCD₂CD₃), and the like.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 5 to 10 carbon atoms. In some embodiments, the aryl group is phenyl or naphthyl. In some embodiments, the aryl is phenyl. The term “aryl” is understood to include deuterated analogs of the aryl groups as defined herein, including but not limited to, groups such as pentadeuterophenyl (i.e., perdeuterophenyl, phenyl-d₅), perdeuteronaphthyl, and the like.

As used herein, “halo” refers to F, Cl, Br, or I. In some embodiments, a halo is F, Cl, or Br. In some embodiments, a halo is F or Cl. In some embodiments, a halo is F. In some embodiments, a halo is Cl.

As used herein, “Cₙ₋ₘ haloalkoxy” refers to a group of formula-O-haloalkyl having n to m carbon atoms. Example haloalkoxy groups include OCF₃ and OCHF₂. In some embodiments, the haloalkoxy group is fluorinated only. In some embodiments, the alkyl group has 1 to 6, 1 to 4, or 1 to 3 carbon atoms. The term “Cₙ₋ₘ haloalkoxy” is understood to include deuterated analogs of the haloalkyl moiety of the haloalkoxy group as defined herein, including but not limited to, groups such as deuterodifluoromethoxy (—OCDF₂), dideuterofluoromethoxy (—OCD₂F), and the like.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like. The term “Cₙ₋ₘ haloalkyl” is understood to include deuterated analogs of the haloalkyl group as defined herein, including but not limited to, groups such as deuterodifluoromethyl (-CDF₂), dideuterofluoromethyl (-CD₂F), and the like.

As used herein, the term “carbonyl”, employed alone or in combination with other terms, refers to a —C(O)— group.

As used herein, the term “Cₙ₋ₘ alkylcarbonyl” refers to a group of formula-C(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula-S(O)₂-alkyl, wherein the alkyl group has n to m carbon atoms. In some embodiments, the alkyl group has 1 to 6, 1 to 4, or 1 to 3 carbon atoms.

As used herein, the term “carboxy” refers to a group of formula-C(O)OH. As used herein, the term “di(Cₙ₋ₘ alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, “cycloalkyl” refers to non-aromatic cyclic hydrocarbons including cyclized alkyl and alkenyl groups. Cycloalkyl groups can include mono- or polycyclic (e.g., having 2 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, azaspiro[2.4]heptanyl, and the like. In some embodiments, cycloalkyl is cyclopropyl, cyclobutyl, cyclopentyl, or cyclohexyl. The term “cycloalkyl” is understood to include deuterated analogs of the cycloalkyl groups as defined herein, including but not limited to, groups such as perdeuterocyclopropyl, perdeuterocyclobutyl, perdeuterocyclopentyl, perdeuterocyclohexyl, and the like.

As used herein, “heteroaryl” refers to a monocyclic or polycyclic (e.g., having 2 fused rings) aromatic heterocycle having at least one heteroatom ring member selected from N, O, S and B. In some embodiments, the heteroaryl ring has 1, 2, 3, or 4 heteroatom ring members independently selected from N, O, S and B. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, S, and B. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-6 monocyclic heteroaryl having 1 or 2 heteroatom ring members independently selected from N, O, S, and B. In some embodiments, the heteroaryl is a 5-6 monocyclic heteroaryl having 1 or 2 heteroatom ring members independently selected from N, O, and S. In some embodiments, the heteroaryl group contains 3 to 10, 4 to 10, 5 to 10, 5 to 7, 3 to 7, or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, thienyl (or thiophenyl), furyl (or furanyl), pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, 1,3,4-oxadiazolyl and 1,2-dihydro-1,2-azaborine, pyridinyl, pyrimidinyl, pyrazinyl, pyridazinyl, azolyl, triazolyl, thiadiazolyl, quinolinyl, isoquinolinyl, indolyl, benzothiophenyl, benzofuranyl, benzisoxazolyl, imidazo[1,2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, triazolo[4,3-a]pyridinyl, 1H-pyrrolo[3,2-b]pyridinyl, 1H-pyrrolo[2,3-b]pyridinyl, pyrazolo[1,5-a]pyridinyl, pyrazolo[1,5-a]pyrimidinyl, indazolyl, imidazo[1,2-b]pyridazinyl, pyrazolo[1,5-a]pyrimidinyl, and the like. The term “heteroaryl” is understood to include deuterated analogs of the heteroaryl groups as defined herein, including but not limited to, groups such as perdeuteropyridinyl, perdeuteropyrazinyl, perdeuteropyrimidinyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, S, and B, and wherein the ring-forming carbon atoms and heteroatoms of a heterocycloalkyl group can be optionally substituted by one or more oxo or sulfido (e.g., C(O), S(O), C(S), or S(O)₂, etc.). When a ring-forming carbon atom or heteroatom of a heterocycloalkyl group is optionally substituted by one or more oxo or sulfide, the O or S of said group is in addition to the number of ring-forming atoms specified herein (e.g., a 1-methyl-6-oxo-1,6-dihydropyridazin-3-yl is a 6-membered heterocycloalkyl group, wherein a ring-forming carbon atom is substituted with an oxo group, and wherein the 6-membered heterocycloalkyl group is further substituted with a methyl group). Heterocycloalkyl groups include monocyclic and polycyclic (e.g., having 2 fused rings) systems. Included in heterocycloalkyl are monocyclic and polycyclic 3 to 10, 4 to 10, 5 to 10, 4 to 7, 5 to 7, or 5 to 6 membered heterocycloalkyl groups. Heterocycloalkyl groups can also include spirocycles and bridged rings (e.g., a 5 to 10 membered bridged biheterocycloalkyl ring having one or more of the ring-forming carbon atoms replaced by a heteroatom independently selected from N, O, S, and B). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. The term “heterocycloalkyl” is understood to include deuterated analogs of the heterocycloalkyl groups as defined herein, including but not limited to, groups such as perdeuteroazetidinyl, perdeuteropyrrolidinyl, perdeuteropiperidinyl, and the like.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl group contains 3 to 10 ring-forming atoms, 4 to 10 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S and B and having one or more oxidized ring members. In some embodiments, the heterocycloalkyl is a monocyclic or bicyclic 5-10 membered heterocycloalkyl having 1, 2, 3, or 4 heteroatoms independently selected from N, O, S, and B and having one or more oxidized ring members. In some embodiments, the heterocycloalkyl is a monocyclic or bicyclic 5 to 10 membered heterocycloalkyl having 1, 2, 3, or 4 heteroatoms independently selected from N, O, and S and having one or more oxidized ring members. In some embodiments, the heterocycloalkyl is a monocyclic 5 to 6 membered heterocycloalkyl having 1, 2, 3, or 4 heteroatoms independently selected from N, O, and S and having one or more oxidized ring members.

Example heterocycloalkyl groups include pyrrolidin-2-one (or 2-oxopyrrolidinyl), 1,3-isoxazolidin-2-one, pyranyl, tetrahydropyranyl, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, 1,2,3,4-tetrahydroisoquinoline, benzazapene, azabicyclo[3.1.0]hexanyl, diazabicyclo[3.1.0]hexanyl, oxobicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxobicyclo[2.2.2]octanyl, azabicyclo[2.2.2]octanyl, azaadamantanyl, diazaadamantanyl, oxo-adamantanyl, azaspiro[3.3]heptanyl, diazaspiro[3.3]heptanyl, oxo-azaspiro[3.3]heptanyl, azaspiro[3.4]octanyl, diazaspiro[3.4]octanyl, oxo-azaspiro[3.4]octanyl, azaspiro[2.5]octanyl, diazaspiro[2.5]octanyl, azaspiro[4.4]nonanyl, diazaspiro[4.4]nonanyl, oxo-azaspiro[4.4]nonanyl, azaspiro[4.5]decanyl, diazaspiro[4.5]decanyl, diazaspiro[4.4]nonanyl, oxo-diazaspiro[4.4]nonanyl, oxo-dihydropyridazinyl, oxo-2,6-diazaspiro[3.4]octanyl, oxohexahydropyrrolo[1,2-a]pyrazinyl, 5,6-dihydro-4H-pyrrolo[1,2-b]pyrazolyl, 3-oxopiperazinyl, oxo-pyrrolidinyl, oxo-pyridinyl, 2,3-dihydrobenzo[b][1,4]dioxinyl, 3,4-dihydro-2H-benzo[b][1,4]oxazinyl, octahydro-2H-pyrido[1,2-a]pyrazinyl, 1,6-diazaspiro[3.3]heptanyl, 3-azabicyclo[3.1.0]hexanyl, 2-oxa-5-azabicyclo[2.2.1]heptanyl, and 2-oxa-5-azabicyclo[4.1.0]heptanyl, and the like.

As used herein, “Cop cycloalkyl-Cₙ₋ₘ alkyl-” refers to a group of formula cycloalkyl-alkylene-, wherein the cycloalkyl has o to p carbon atoms and the alkylene linking group has n to m carbon atoms.

As used herein “Cₒ₋ₚ aryl-Cₙ₋ₘ alkyl-” refers to a group of formula aryl-alkylene-, wherein the aryl has o to p carbon atoms and the alkylene linking group has n to m carbon atoms.

As used herein, “heteroaryl-Cₙ₋ₘ alkyl-” refers to a group of formula heteroaryl-alkylene-, wherein alkylene linking group has n to m carbon atoms.

As used herein “heterocycloalkyl-Cₙ₋ₘ alkyl-” refers to a group of formula heterocycloalkyl-alkylene-, wherein alkylene linking group has n to m carbon atoms.

As used herein, an “alkyl linking group” is a bivalent straight chain or branched alkyl linking group (“alkylene group”). For example, “Cop cycloalkyl-Cₙ₋ₘ alkyl-”, “Cₒ₋ₚ aryl-Cₙ₋ₘ alkyl-”, “phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-dilyl, propan-1,2-diyl, propan-1,1-diyl and the like. The terms “alkyl linking group” and “alkylene linking group” are understood to include deuterated analogs of the alkylene groups as defined herein.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl, or sulfonyl group.

As used herein, the term “independently selected from” means that each occurrence of a variable or substituent (e.g., each R^{M}), are independently selected at each occurrence from the applicable list.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula I, Formula Ia, etc.) provided herein include stereoisomers of the compounds.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

As will be appreciated by those skilled in the art, the compounds provided herein, including salts and stereoisomers thereof, can be prepared using known organic synthesis techniques and can be synthesized according to any of numerous possible synthetic routes.

Compounds of Formula (I) can be prepared, for example, using a process as illustrated in Scheme 1. In the process depicted in Scheme 1, the compounds of formula 1-1, where L₁ is a halogen (e.g., F, Cl, or Br), can react with the compounds 1-2 via nucleophilic aromatic substitution reactions (e.g., in the presence of a base, such as N,N-diisopropylethylamine) followed by reduction of the nitro group in compound 1-3 (e.g. under reductive conditions, such as treatment with Zn powder, H₂O and NH₄Cl) resulting in the formation of the compounds of formula 1-4. The compounds of formula 1-4 can be converted to cyclic urea 1-5 under standard conditions (e.g., in the presence of N-succinimidyl carbonate). The compounds of formula 1-5 can be further transformed to the compounds of formula 1-6 via C—N bond formation (e.g., in the presence of an alkyl halide (e.g., iodomethane) and base (e.g., (cesium carbonate)). The compounds of formula 1-6, where L₂ is a halogen (e.g., Cl, or Br), are further joined together with the compounds of formula 1-7 via Buchwald-Hartwig cross-coupling reactions (e.g., in the presence of tris(dibenzylideneacetone)dipalladium(0), Xantphos and cesium carbonate) to yield the compounds of Formula I.

Alternatively, the compounds of formula 1-6 can be used to prepare compounds of formula 1-8 through Buchwald-Hartwig cross-coupling reactions (e.g., in the presence of tris(dibenzylideneacetone)dipalladium(0), BrettPhos and cesium carbonate) followed by NH deprotection of the protecting group (e.g., treatment with HCl or TFA). Then, the compounds of formula 1-8 can be coupled with the compounds of formula 1-9, where X is a halogen (e.g., Cl, or Br), under Buchwald-Hartwig cross-coupling reactions (e.g., in the presence of tris(dibenzylideneacetone)dipalladium(0), Xantphos and cesium carbonate) to afford the compounds of Formula I.

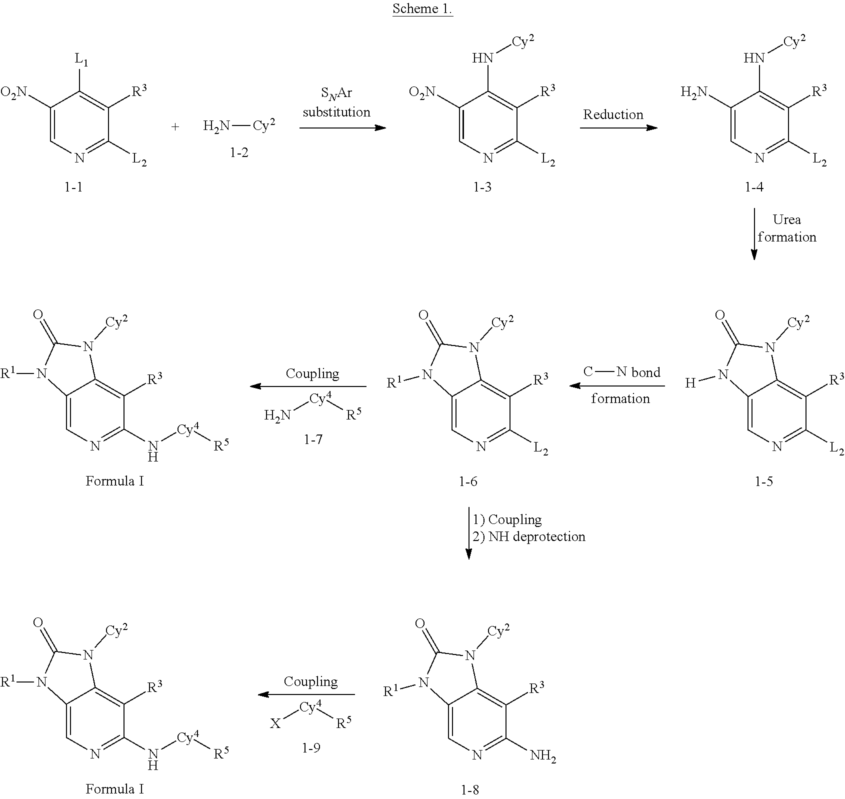

When Cy⁴ is a 6-membered aryl or heteroaryl ring, the compounds of formula 1-7 and 1-9 can be prepared using a process as illustrated in Scheme 2. The compounds of formula 2-1, where X is a halogen (e.g., Cl, Br, or I), can react with the compounds 2-2 (M is B(OR)₂, Sn(alkyl)₃, Zn-hal, etc.) under standard Suzuki cross-coupling conditions (e.g., in the presence of a palladium catalyst and a suitable ligand/base), or standard Stille cross-coupling conditions (e.g., in the presence of a palladium catalyst and a suitable ligand), or standard Negishi cross-coupling conditions (e.g., in the presence of a palladium catalyst and a suitable ligand), to give compounds of formula 2-3. After functional group manipulations (e.g., amide coupling, cross coupling, Grignard addition etc.) on the compound 2-3, the compounds of formula 1-7 can be prepared.

Alternatively, the compounds of formula 2-4, where X is a halogen (e.g., Cl, Br, or I), can be converted to the compounds 2-5 (M is B(OR)₂, Sn(alkyl)₃, etc.) under standard conditions (e.g., in the presence of a palladium catalyst and a suitable ligand). The compounds of formula 2-5 can be further transformed to the compounds of formula 2-7 (1-9) via cross-coupling reaction with the compounds of formula 2-6 under standard conditions (e.g., in the presence of a palladium catalyst and a suitable ligand/base). After functional group manipulations (e.g., amide coupling, cross coupling, Grignard addition etc.) on the compound 2-7, the compounds of formula 1-7 can be prepared.

The compounds of formula 1-9, can be also prepared from the compounds of formula 2-4, where X is a halogen (e.g., Cl, Br, or I), through nucleophilic aromatic substitution reactions with the compounds 2-8 (e.g., in the presence of a base, such as N,N-diisopropylethylamine) followed by functional group manipulations (e.g., amide coupling, cross coupling, Grignard addition etc.) on the compound 2-9. In Scheme 2, at least one RR group corresponds to variable R⁵ as defined herein and each additional RR group corresponds to H or variable R⁴ as defined herein.

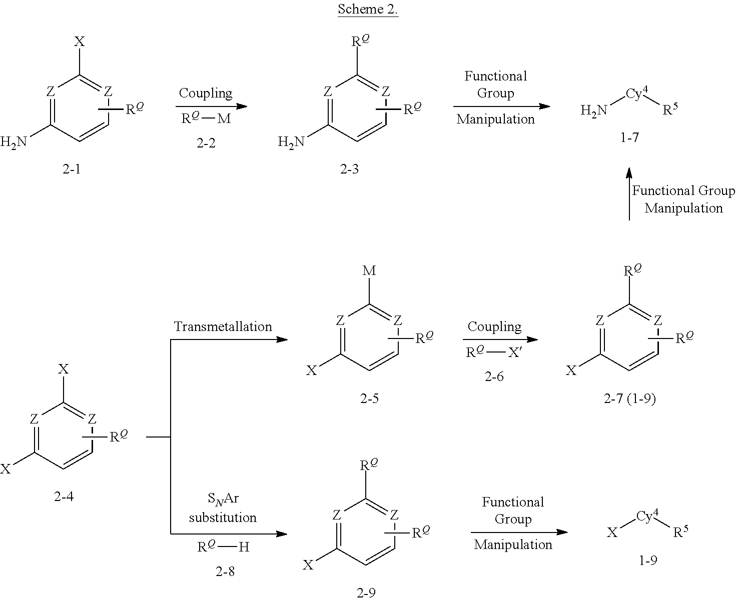

When Cy⁴ is 1H-pyrrolo[3,2-b]pyridine or 1H-pyrazolo[4,3-b]pyridine ring, the compounds of formula 1-7 and 1-9 can be prepared using a process as illustrated in Scheme 3. The compounds of formula 3-1 can undergo a halogenation reaction (e.g., in the presence of N-iodosuccinimide) followed by alkylation reaction with the compound 2-2 (L is halogens, OMs, etc.) to provide the compounds of formula 3-3. The compounds of formula 3-3 can react with the compounds 3-4 (e.g., M is B(OR)₂) under standard cross-coupling conditions (e.g., in the presence of a palladium catalyst and a suitable ligand/base) to give a derivative of formula 3-5. After functional group manipulations (e.g., amide coupling, cross coupling, 10 Grignard addition etc.) on the compound 3-5, the compounds of formula 1-7 (if Y=NHPG) and 1-9 (if Y=halogens) can be prepared. In Scheme 3, at least one R^{Q} group corresponds to variable R⁵ as defined herein and each additional R^{Q} group corresponds to H or variable R⁴ as defined herein.

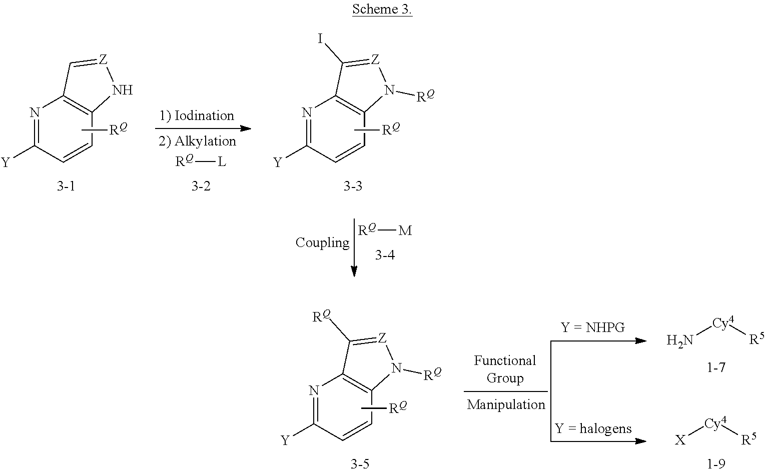

When Cy⁴ is 3H-imidazo[4,5-b]pyridine or 1,3-dihydro-2H-imidazo[4,5-b]pyridin-2-one ring, the compounds of formula 1-9 can be prepared using a process as illustrated in Scheme 4. The compounds of formula 4-1, where L₁ is a halogen (e.g., F, Cl, or Br), can react with the compounds 4-2 via nucleophilic aromatic substitution reactions (e.g., in the presence of a base, such as N,N-diisopropylethylamine) to give the compounds of formula 4-3. The reduction of the nitro group in compound 4-3 (e.g. under reductive conditions, such as treatment with Zn powder, H₂O and NH₄Cl) followed by cyclization reaction (e.g. in the presence of CDI or methyl orthoformate) yield the compounds of formula 4-4. After 10 functional group manipulations (e.g., amide coupling, cross coupling, Grignard addition etc.) on the compound 4-4, the compounds of 1-9 can be prepared. In Scheme 4, at least one R^{Q} group corresponds to variable R⁵ as defined herein and each additional R^{Q} group corresponds to H or variable R⁴ as defined herein.

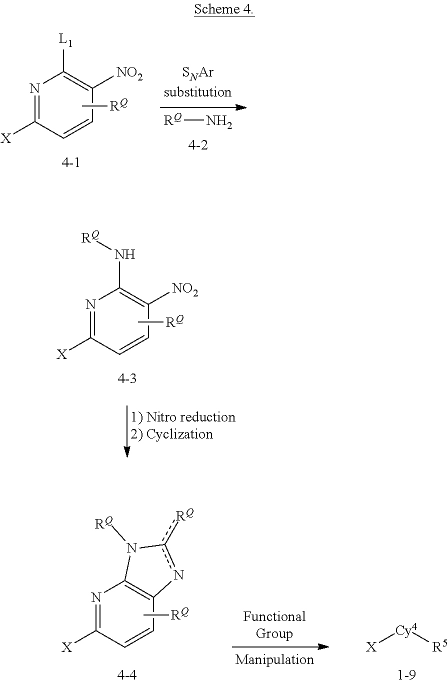

When Cy⁴ is imidazo[1,2-b]pyridazine ring, the compounds of formula 1-7 can be prepared using a process as illustrated in Scheme 5. The compounds of formula 5-1 can couple with the compounds 5-2 (e.g., M is B(OR)₂ Zn-hal, etc.) under standard Suzuki cross-coupling conditions (e.g., in the presence of a palladium catalyst and a suitable ligand/base), or standard Negishi cross-coupling conditions (e.g., in the presence of a palladium catalyst and a suitable ligand), to generate the compound of formula 5-3. Iodination of the compound 5-3 (e.g., in the presence of N-iodosuccinimide) affords the compound of formula 5-4. The compounds of formula 5-4 can react with the compounds 5-5 (e.g., M is B(OR)₂) under standard cross-coupling conditions (e.g., in the presence of a palladium catalyst and a suitable ligand/base) to give compounds of formula 5-6 (1-9). The compounds of formula 5-6 can be elaborated into the compounds of formula 1-7 through Buchwald-Hartwig cross-coupling reactions (e.g., in the presence of a palladium catalyst and a suitable ligand) followed by NH deprotection of the protecting group (e.g., treatment with HCl or TFA). In Scheme 5, at least one R^{Q} group corresponds to variable R⁵ as defined herein and each additional R^{Q} group corresponds to H or variable R⁴ as defined herein . . .

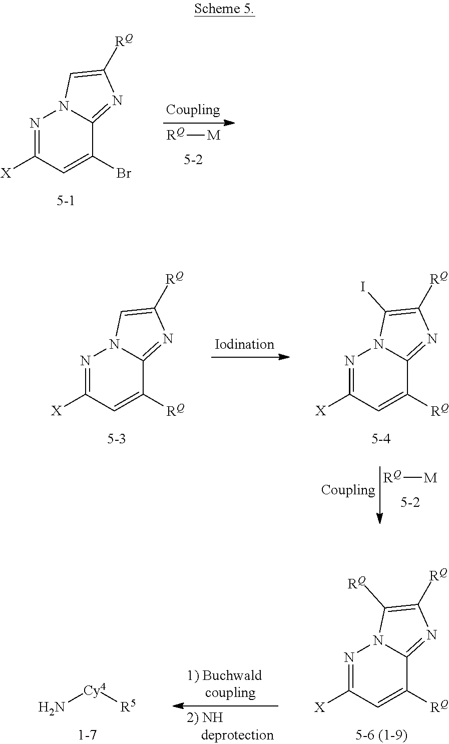

The reactions for preparing compounds described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, (e.g., temperatures which can range from the solvent's freezing temperature to the solvent's boiling temperature). A given reaction can be carried out in one solvent or a mixture of more than one solvent. Depending on the particular reaction step, suitable solvents for a particular reaction step can be selected by the skilled artisan.

The expressions, “ambient temperature” or “room temperature” or “rt” as used herein, are understood in the art, and refer generally to a temperature, e.g., a reaction temperature, that is about the temperature of the room in which the reaction is carried out, for example, a temperature from about 20° C. to about 30° C.

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

### Methods of Use

The compounds described herein can inhibit the activity of the V617F variant of the protein-tyrosine kinase JAK2 (i.e., “V617F” or “JAK2V617F”). Compounds which inhibit V617F are useful in providing a means of preventing the growth or inducing apoptosis in tumors, particularly by inhibiting angiogenesis. It is therefore anticipated that the compounds of the disclosure are useful in treating or preventing proliferative disorders such as cancers. In particular tumors with activating mutants of receptor tyrosine kinases or upregulation of receptor tyrosine kinases may be particularly sensitive to the inhibitors.

In certain embodiments, the disclosure provides a method for treating a V617F-related disorder in a patient in need thereof, comprising the step of administering to said patient a compound of the disclosure, or a pharmaceutically acceptable composition thereof.

Myeloproliferative diseases (MPD) are multipotent hematopoietic stem cell disorders characterized by excess production of various blood cells. MPNs include polycythemia vera (PV), essential thrombocythemia (ET), and idiopathic myelofibrosis (IMF). JAK2 V617F mutation is reported in about 95% of patients with PV, in 35% to 70% of patients with ET, and 50% of patients with IMF. Also, JAK2 exon 12 mutations are detected in some of the V617F-negative PV patients (Ma et al., J. Mol. Diagn., 11: 49-53, 2009). In some embodiments, the compounds of the disclosure can be useful in the treatment of myeloproliferative disorders (e.g., myeloproliferative neoplasms) in a patient in need thereof, such as polycythemia vera, essential thrombocythemia, myelofibrosis with myeloid metaplasia (MMM), primary myelofibrosis (PMF), chronic myelogenous leukemia (CML), chronic myelomonocytic leukemia (CMML), hypereosinophilic syndrome (HES), systemic mast cell disease (SMCD), and the like.

In some embodiments, the myeloproliferative disorder is selected from polycythemia vera, essential thrombocythemia, myelofibrosis with myeloid metaplasia, primary myelofibrosis, post-essential thrombocythemia myelofibrosis, and post polycythemia vera myelofibrosis.

In some embodiments, the myeloproliferative disorder is a myeloproliferative neoplasm.

In some embodiments, the myeloproliferative disorder is myelofibrosis (e.g., primary myelofibrosis (PMF) or post polycythemia vera/essential thrombocythemia myelofibrosis (Post-PV/ET MF)).

In some embodiments, the myeloproliferative disorder is primary myelofibrosis (PMF).

In some embodiments, the myeloproliferative disorder is post-essential thrombocythemia myelofibrosis (Post-ET MF).

In some embodiments, the myeloproliferative disorder is post polycythemia vera myelofibrosis (Post-PV MF).

In some embodiments, the myeloproliferative disorder is selected from primary myelofibrosis (PMF), polycythemia vera (PV), and essential thrombocythemia (ET).

In some embodiments, the myeloproliferative neoplasm is primary myelofibrosis (PMF).

In some embodiments, the myeloproliferative neoplasm is polycythemia vera (PV).

In some embodiments, the myeloproliferative neoplasm is essential thrombocythemia (ET).

Myeloproliferative diseases include disorders of a bone marrow or lymph node-derived cell type, such as a white blood cell. A myeloproliferative disease can manifest by abnormal cell division resulting in an abnormal level of a particular hematological cell population. The abnormal cell division underlying a proliferative hematological disorder is typically inherent in the cells and not a normal physiological response to infection or inflammation. Leukemia is a type of myeloproliferative disease. Exemplary myeloproliferative diseases include, but are not limited to, acute myeloid leukemia (AML), acute lymphoblastic leukemia (ALL), chronic lymphocytic leukemia (CLL), myelodysplastic syndrome (MDS), chronic myeloid leukemia (CML), hairy cell leukemia, leukemic manifestations of lymphomas, multiple myeloma, polycythemia vera (PV), essential thrombocythemia (ET), idiopathic myelofibrosis (IMF), hypereosinophilic syndrome (HES), chronic neutrophilic leukemia (CNL), myelofibrosis with myeloid metaplasia (MMM), chronic myelomonocytic leukemia (CMML), juvenile myelomonocytic leukemia, chronic basophilic leukemia, chronic eosinophilic leukemia, systemic mastocytosis (SM), and unclassified myeloproliferative diseases (UMPD or MPD-NC). Lymphoma is a type of proliferative disease that mainly involves lymphoid organs, such as lymph nodes, liver, and spleen. Exemplary proliferative lymphoid disorders include lymphocytic lymphoma (also called chronic lymphocytic leukemia), follicular lymphoma, large cell lymphoma, Burkitt's lymphoma, marginal zone lymphoma, lymphoblastic lymphoma (also called acute lymphoblastic lymphoma).

For example, the compounds of the disclosure are useful in the treatment of cancer. Example cancers include bladder cancer (e.g., urothelial carcinoma, squamous cell carcinoma, adenocarcinoma), breast cancer (e.g., hormone R positive, triple negative), cervical cancer, colorectal cancer, cancer of the small intestine, colon cancer, rectal cancer, cancer of the anus, endometrial cancer, gastric cancer (e.g., gastrointestinal stromal tumors), head and neck cancer (e.g., cancers of the larynx, hypopharynx, nasopharynx, oropharynx, lips, and mouth, squamous head and neck cancers), kidney cancer (e.g., renal cell carcinoma, urothelial carcinoma, sarcoma, Wilms tumor), liver cancer (e.g., hepatocellular carcinoma, cholangiocellular carcinoma (e.g., intrahepatic, hilar or perihilar, distal extrahepatic), liver angiosarcoma, hepatoblastoma), lung cancer (e.g., adenocarcinoma, small cell lung cancer and non-small cell lung carcinomas, parvicellular and non-parvicellular carcinoma, bronchial carcinoma, bronchial adenoma, pleuropulmonary blastoma), ovarian cancer, prostate cancer, testicular cancer, uterine cancer, vulvar cancer, esophageal cancer, gall bladder cancer, pancreatic cancer (e.g. exocrine pancreatic carcinoma), stomach cancer, thyroid cancer, parathyroid cancer, neuroendocrine cancer (e.g., pheochromocytoma, Merkel cell cancer, neuroendocrine carcinoma), skin cancer (e.g., squamous cell carcinoma, Kaposi sarcoma, Merkel cell skin cancer), and brain cancer (e.g., astrocytoma, medulloblastoma, ependymoma, neuro-ectodermal tumors, pineal tumors).

Further example cancers include hematopoietic malignancies such as leukemia or lymphoma, multiple myeloma, chronic lymphocytic lymphoma, adult T cell leukemia, acute myeloid leukemia (AML), B-cell lymphoma, cutaneous T-cell lymphoma, acute myelogenous leukemia, Hodgkin's or non-Hodgkin's lymphoma, myeloproliferative neoplasms (e.g., 8p11 myeloproliferative syndrome, polycythemia vera (PV), essential thrombocythemia (ET), and primary myelofibrosis (PMF)), myelodysplastic syndrome, chronic cosinophilic leukemia, Waldenstrom's Macroglubulinemia, hairy cell lymphoma, chronic myelogenic lymphoma, acute lymphoblastic lymphoma, AIDS-related lymphomas, and Burkitt's lymphoma.

In certain embodiments, provided herein is a method of treating cancer comprising administering to a patient in need thereof a therapeutically effect amount of a compound of the disclosure. In certain embodiments, the cancer is selected from T lymphoblastic lymphoma, glioblastoma, melanoma, rhabdosarcoma, lymphosarcoma, and osteosarcoma.

Other cancers treatable with the compounds of the disclosure include tumors of the eye, glioblastoma, melanoma, leiomyosarcoma, and urothelial carcinoma (e.g., ureter, urethra, bladder, urachus).

The compounds of the disclosure can also be useful in the inhibition of tumor metastases.

In some embodiments, the compounds of the disclosure as described herein can be used to treat Alzheimer's disease, HIV, or tuberculosis.

In some embodiments, the compounds of the disclosure can be useful in the treatment of myelodysplastic syndrome (MDS) in a patient in need thereof. In some embodiments, said patient having the myelodysplastic syndrome (MDS) is red blood cell transfusion dependent.

As used herein, myelodysplastic syndromes are intended to encompass heterogeneous and clonal hematopoietic disorders that are characterized by ineffective hematopoiesis on one or more of the major myeloid cell lineages. Myelodysplastic syndromes are associated with bone marrow failure, peripheral blood cytopenias, and a propensity to progress to acute myeloid leukemia (AML). Moreover, clonal cytogenetic abnormalities can be detected in about 50% of cases with MDS. In 1997, The World Health Organization (WHO) in conjunction with the Society for Hematopathology (SH) and the European Association of Hematopathology (EAHP) proposed new classifications for hematopoietic neoplasms (Harris, et al., J Clin Oncol 1999; 17:3835-3849; Vardiman, et al., Blood 2002; 100:2292-2302). For MDS, the WHO utilized not only the morphologic criteria from the French-American-British (FAB) classification but also incorporated available genetic, biologic, and clinical characteristics to define subsets of MDS (Bennett, et al., Br. J. Haematol. 1982; 51:189-199). In 2008, the WHO classification of MDS (Table 1) was further refined to allow precise and prognostically relevant subclassification of unilineage dysplasia by incorporating new clinical and scientific information (Vardiman, et al., Blood 2009; 114:937-951; Swerdlow, et al., WHO Classification of Tumours of Haematopoietic and Lymphoid Tissues. 4th Edition. Lyon France: IARC Press; 2008:88-103; Bunning and Germing, “Myelodysplastic syndromes/neoplasms” in Chapter 5, Swerdlow, et al, eds. WHO Classification of Tumours of Haematopoietic and Lymphoid Tissues. (ed. 4th edition): Lyon, France: IARC Press; 2008:88-103).

TABLE 1
2008 WHO Classification for De Novo Myelodysplastic Syndrome

| Subtype | Blood | Bone Marrow |
|---|---|---|
| Refractory cytopenia with | Single or Bicytopenia | Dysplasia in ≥10% of 1 cell |
| unilineage dysplasia | | line, <5% blasts |
| (RCUD) | | |
| Refractory anemia with | Anemia, no blasts | ≥15% of erythroid precursors |
| ring sideroblasts (RARS) | | w/ring sideroblasts, erythroid |
| | | dysplasia only, <5% blasts |
| Refractory cytopenia with | Cytopenia(s), <1 × 10⁹/L | Dysplasia in ≥10% of cells in ≥2 |
| multilineage dysplasia | monocytes | hematopoietic lineages, ±15% |
| | | ring sideroblasts, <5% blasts |
| Refractory anemia with | Cytopenia(s), ≤2% to | Unilineage or multilineage |
| excess blasts-1 (RAEB-1) | 4% blasts, <1 × 10⁹/L | dysplasia, No Auer rods, 5% to |
| | monocytes | 9% blasts |
| Refractory anemia with | Cytopenia(s), ≤5% to | Unilineage or multilineage |
| excess blasts-2 (RAEB-2) | 19% blasts, <1 × 10⁹/L | dysplasia, ±Auer rods, 10% to |
| | monocytes | 19% blasts |
| Myelodysplastic syndrome, | Cytopenias | Unilineage or no dysplasia but |
| unclassified (MDS-U) | | characteristic MDS |
| | | cytogenetics, <5% blasts |
| MDS associated with | Anemia, platelets normal | Unilineage erythroid. Isolated |
| isolated del(5q) | or increased | del(5q), <5% blasts |

In some embodiments, the myelodysplastic syndrome is refractory cytopenia with unilineage dysplasia (RCUD).

In some embodiments, the myelodysplastic syndrome is refractory anemia with ring sideroblasts (RARS).

In some embodiments, the myelodysplastic syndrome is refractory anemia with ring sideroblasts associated with thrombocytosis (RARS-T).

In some embodiments, the myelodysplastic syndrome is refractory cytopenia with multilineage dysplasia.

In some embodiments, the myelodysplastic syndrome is refractory anemia with excess blasts-1 (RAEB-1).

In some embodiments, the myelodysplastic syndrome is refractory anemia with excess blasts-2 (RAEB-2).

In some embodiments, the myelodysplastic syndrome is myelodysplastic syndrome, unclassified (MDS-U).

In some embodiments, the myelodysplastic syndrome is myelodysplastic syndrome associated with isolated del(5q).

In some embodiments, the myelodysplastic syndrome is refractory to erythropoiesis-stimulating agents.

In some embodiments, the compounds of the disclosure can be useful in the treatment of myeloproliferative disorder/myelodysplastic overlap syndrome (MPD/MDS overlap syndrome).

In some embodiments, the compounds of the disclosure can be useful in the treatment of leukemia.

In some embodiments, the compounds of the disclosure can be useful in the treatment of acute myeloid leukemia (AML).

In addition to oncogenic neoplasms, the compounds of the disclosure can be useful in the treatment of skeletal and chondrocyte disorders including, but not limited to, achrondroplasia, hypochondroplasia, dwarfism, thanatophoric dysplasia (TD) (clinical forms TD I and TD II), Apert syndrome, Crouzon syndrome, Jackson-Weiss syndrome, Beare-Stevenson cutis gyrate syndrome. Pfeiffer syndrome, and craniosynostosis syndromes.

The compounds provided herein may further be useful in the treatment of fibrotic diseases, such as where a disease symptom or disorder is characterized by fibrosis. Example fibrotic diseases include liver cirrhosis, glomerulonephritis, pulmonary fibrosis, systemic fibrosis, rheumatoid arthritis, and wound healing.

In some embodiments, the compounds provided herein can be used in the treatment of a hypophosphatemia disorder such as, for example, X-linked hypophosphatemic rickets, autosomal recessive hypophosphatemic rickets, and autosomal dominant hypophosphatemic rickets, or tumor-induced osteromalacia.

In some embodiments, provided herein is a method of increasing survival or progression-free survival in a patient, comprising administering a compound provided herein to the patient. In some embodiments, the patient has cancer. In some embodiments, the patient has a disease or disorder described herein. As used herein, progression-free survival refers to the length of time during and after the treatment of a solid tumor that a patient lives with the disease but it does not get worse. Progression-free survival can refer to the length of time from first administering the compound until the earlier of death or progression of the disease. Progression of the disease can be defined by RECIST v. 1.1 (Response Evaluation Criteria in Solid Tumors), as assessed by an independent centralized radiological review committee. In some embodiments, administering of the compound results in a progression free survival that is greater than about 1 month, about 2 months, about 3 months, about 4 months, about 5 months, about 6 months, about 8 months, about 9 months, about 12 months, about 16 months, or about 24 months. In some embodiments, the administering of the compound results in a progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months. In some embodiments, the administering of the compound results in an increase of progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months.

The present disclosure further provides a compound described herein, or a pharmaceutically acceptable salt thereof, for use in any of the methods described herein.

The present disclosure further provides use of a compound described herein, or a pharmaceutically acceptable salt thereof, for the preparation of a medicament for use in any of the methods described herein.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” a V617F variant with a compound described herein includes the administration of a compound described herein to an individual or patient, such as a human, having a V617F variant, as well as, for example, introducing a compound described herein into a sample containing a cellular or purified preparation containing the V617F variant.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients. 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, the term “treating” or “treatment” refers to inhibiting the disease; for example, inhibiting a disease, condition or disorder in an individual who is experiencing or displaying the pathology or symptomatology of the disease, condition or disorder (i.e., arresting further development of the pathology and/or symptomatology) or ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

### Combination Therapies

One or more additional pharmaceutical agents or treatment methods such as, for example, anti-viral agents, chemotherapeutics or other anti-cancer agents, immune enhancers, immunosuppressants, radiation, anti-tumor and anti-viral vaccines, cytokine therapy (e.g., IL2, GM-CSF, etc.), and/or tyrosine kinase inhibitors can be used in combination with compounds described herein for treatment or prevention of V617F-associated diseases, disorders or conditions, or diseases or conditions as described herein. The agents can be combined with the present compounds in a single dosage form, or the agents can be administered simultaneously or sequentially as separate dosage forms.

Compounds described herein can be used in combination with one or more other kinase inhibitors for the treatment of diseases, such as cancer, that are impacted by multiple signaling pathways. For example, a combination can include one or more inhibitors of the following kinases for the treatment of cancer: Akt1, Akt2, Akt3, TGF-βR, Pim, PKA, PKG, PKC, CaM-kinase, phosphorylase kinase, MEKK, ERK, MAPK, mTOR, EGFR, HER2, HER3, HER4, INS-R, IGF-1R, IR-R, PDGFαR, PDGFβR, CSFIR, KIT, FLK-II, KDR/FLK-1, FLK-4, flt-1, FGFR1, FGFR2, FGFR3, FGFR4, c-Met, Ron, Sca, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. Additionally, the solid forms of the inhibitor as described herein can be combined with inhibitors of kinases associated with the PIK3/Akt/mTOR signaling pathway, such as PI3K, Akt (including Akt1, Akt2 and Akt3) and mTOR kinases.

In some embodiments, compounds described herein can be used in combination with one or more inhibitors of the enzyme or protein receptors such as HPK1, SBLB, TUT4, A2A/A2B, CD19, CD47, CDK2, STING, ALK2, LIN28, ADAR1, MAT2a, RIOK1, HDAC8, WDR5, SMARCA2, and DCLK1 for the treatment of diseases and disorders. Exemplary diseases and disorders include cancer, infection, inflammation and neurodegenerative disorders.

In some embodiments, compounds described herein can be used in combination with a therapeutic agent that targets an epigenetic regulator. Examples of epigenetic regulators include bromodomain inhibitors, the histone lysine methyltransferases, histone arginine methyl transferases, histone demethylases, histone deacetylases, histone acetylases, and DNA methyltransferases. Histone deacetylase inhibitors include, e.g., vorinostat.

For treating cancer and other proliferative diseases, compounds described herein can be used in combination with targeted therapies, including JAK kinase inhibitors (ruxolitinib, additional JAK1/2 and JAK1-selective, baricitinib or itacitinib), Pim kinase inhibitors (e.g., LGH447, INCB053914 and SGI-1776), PI3 kinase inhibitors including PI3K-delta selective and broad spectrum PI3K inhibitors (e.g., INCB50465 and INCB50797), PI3K-gamma inhibitors such as PI3K-gamma selective inhibitors, MEK inhibitors, CSFIR inhibitors (e.g., PLX3397 and LY3022855), TAM receptor tyrosine kinases inhibitors (Tyro-3, Axl, and Mer; e.g., INCB81776), angiogenesis inhibitors, interleukin receptor inhibitors. Cyclin Dependent kinase inhibitors, BRAF inhibitors, mTOR inhibitors, proteasome inhibitors (Bortezomib, Carfilzomib), HDAC-inhibitors (panobinostat, vorinostat), DNA methyl transferase inhibitors, dexamethasone, bromo and extra terminal family members inhibitors (for example, bromodomain inhibitors or BET inhibitors, such as OTX015, CPI-0610, INCB54329 or INCB57643), LSD1 inhibitors (e.g., GSK2979552, INCB59872 and INCB60003), arginase inhibitors (e.g., INCB1158), indoleamine 2,3-dioxygenase inhibitors (e.g., epacadostat, NLG919 or BMS-986205), PARP inhibiors (e.g., olaparib or rucaparib), and inhibitors of BTK such as ibrutinib.

For treating cancer and other proliferative diseases, compounds described herein can be used in combination with chemotherapeutic agents, agonists or antagonists of nuclear receptors, or other anti-proliferative agents. Compounds described herein can also be used in combination with a medical therapy such as surgery or radiotherapy, e.g., gamma-radiation, neutron beam radiotherapy, electron beam radiotherapy, proton therapy, brachytherapy, and systemic radioactive isotopes.

Examples of suitable chemotherapeutic agents include any of: abarelix, abiraterone, afatinib, aflibercept, aldesleukin, alemtuzumab, alitretinoin, allopurinol, altretamine, amidox, amsacrine, anastrozole, aphidicolon, arsenic trioxide, asparaginase, axitinib, azacitidine, bevacizumab, bexarotene, baricitinib, bendamustine, bicalutamide, bleomycin, bortezombi, bortezomib, brivanib, buparlisib, busulfan intravenous, busulfan oral, calusterone, camptosar, capecitabine, carboplatin, carmustine, cediranib, cetuximab, chlorambucil, cisplatin, cladribine, clofarabine, crizotinib, cyclophosphamide, cytarabine, dacarbazine, dacomitinib, dactinomycin, dalteparin sodium, dasatinib, dactinomycin, daunorubicin, decitabine, degarelix, denileukin, denileukin diftitox, deoxycoformycin, dexrazoxane, didox, docetaxel, doxorubicin, droloxafine, dromostanolone propionate, eculizumab, enzalutamide, epidophyllotoxin, epirubicin, epothilones, erlotinib, estramustine, etoposide phosphate, etoposide, exemestane, fentanyl citrate, filgrastim, floxuridine, fludarabine, fluorouracil, flutamide, fulvestrant, gefitinib, gemcitabine, gemtuzumab ozogamicin, goserelin acetate, histrelin acetate, ibritumomab tiuxetan, idarubicin, idelalisib, ifosfamide, imatinib mesylate, interferon alfa 2a, irinotecan, lapatinib ditosylate, lenalidomide, letrozole, leucovorin, leuprolide acetate, levamisole, lonafarnib, lomustine, meclorethamine, megestrol acetate, melphalan, mercaptopurine, methotrexate, methoxsalen, mithramycin, mitomycin C, mitotane, mitoxantrone, nandrolone phenpropionate, navelbene, necitumumab, nelarabine, neratinib, nilotinib, nilutamide, niraparib, nofetumomab, oserelin, oxaliplatin, paclitaxel, pamidronate, panitumumab, panobinostat, pazopanib, pegaspargase, pegfilgrastim, pemetrexed disodium, pentostatin, pilaralisib, pipobroman, plicamycin, ponatinib, porfimer, prednisone, procarbazine, quinacrine, ranibizumab, rasburicase, regorafenib, reloxafine, revlimid, rituximab, rucaparib, ruxolitinib, sorafenib, streptozocin, sunitinib, sunitinib maleate, tamoxifen, tegafur, temozolomide, teniposide, testolactone, tezacitabine, thalidomide, thioguanine, thiotepa, tipifarnib, topotecan, toremifene, tositumomab, trastuzumab, tretinoin, triapine, trimidox, triptorelin, uracil mustard, valrubicin, vandetanib, vinblastine, vincristine, vindesine, vinorelbine, vorinostat, veliparib, talazoparib, and zoledronate.

In some embodiments, compounds described herein can be used in combination with immune checkpoint inhibitors. Exemplary immune checkpoint inhibitors include inhibitors against immune checkpoint molecules such as CD27, CD28, CD40, CD122, CD96, CD73, CD47, OX40, GITR, CSFIR, JAK, PI3K delta, PI3K gamma, TAM, arginase, CD137 (also known as 4-1BB), ICOS, A2AR, B7-H3, B7-H4, BTLA, CTLA-4, LAG3 (e.g., INCAGN2385), TIM3 (e.g., INCB2390), VISTA, PD-1, PD-L1 and PD-L2. In some embodiments, the immune checkpoint molecule is a stimulatory checkpoint molecule selected from CD27, CD28, CD40, ICOS, OX40 (e.g., INCAGN1949), GITR (e.g., INCAGN1876) and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is a small molecule PD-L1 inhibitor. In some embodiments, the small molecule PD-L1 inhibitor has an IC50 less than 1 μM, less than 100 nM, less than 10 nM or less than 1 nM in a PD-L1 assay described in US Patent Publication Nos. US 20170107216, US 20170145025, US 20170174671, US 20170174679, US 20170320875, US 20170342060, US 20,170,362253, and US 20180016260, each of which is incorporated by reference in its entirety for all purposes.

In some embodiments, the inhibitor of an immune checkpoint molecule is an inhibitor of PD-1, e.g., an anti-PD-1 monoclonal antibody. In some embodiments, the anti-PD-1 monoclonal antibody is retifanlimab (also known as MGA012), nivolumab, pembrolizumab (also known as MK-3475), pidilizumab, SHR-1210, PDR001, ipilumimab or AMP-224. In some embodiments, the anti-PD-1 monoclonal antibody is nivolumab or pembrolizumab. In some embodiments, the anti-PD1 antibody is pembrolizumab. In some embodiments, the anti-PD1 antibody is nivolumab. In some embodiments, the anti-PD-1 monoclonal antibody is retifanlimab. In some embodiments, the anti-PD1 antibody is SHR-1210. Other anti-cancer agent(s) include antibody therapeutics such as 4-1BB (e.g. urelumab, utomilumab.

In some embodiments, the compounds of the disclosure can be used in combination with INCB086550.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A or MEDI4736.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, or INCAGN2385.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of GITR, e.g., an anti-GITR antibody. In some embodiments, the anti-GITR antibody is TRX518, MK-4166, INCAGN1876, MK-1248, AMG228, BMS-986156, GWN323, or MEDI1873.

In some embodiments, the inhibitor of an immune checkpoint molecule is an agonist of OX40, e.g., OX40 agonist antibody or OX40L fusion protein. In some embodiments, the anti-OX40 antibody is MEDI0562, MOXR-0916, PF-04518600, GSK3174998, or BMS-986178. In some embodiments, the OX40L fusion protein is MEDI6383.

In some embodiments, the inhibitor of an immune checkpoint molecule is an inhibitor of CD20, e.g., an anti-CD20 antibody. In some embodiments, the anti-CD20 antibody is obinutuzumab or rituximab.

The compounds of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFβ receptor.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196.

In some embodiments, the compounds described herein can be used in combination with one or more agents for the treatment of diseases such as cancer. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

Suitable antiviral agents contemplated for use in combination with compounds of the present disclosure can comprise nucleoside and nucleotide reverse transcriptase inhibitors (NRTIs), non-nucleoside reverse transcriptase inhibitors (NNRTIs), protease inhibitors and other antiviral drugs.

Example suitable NRTIs include zidovudine (AZT); didanosine (ddl); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(−)-FTC]; beta-L-FD4 (also called beta-L-D4C and named beta-L-2′, 3′-dicleoxy-5-fluoro-cytidene); DAPD, ((−)-beta-D-2,6,-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrimidinedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; BMS-2322623; ABT-378; and AG-1 549. Other antiviral agents include hydroxyurea, ribavirin, IL-2, IL-12, pentafuside and Yissum Project No. 11607.

Suitable agents for use in combination with compounds described herein for the treatment of cancer include chemotherapeutic agents, targeted cancer therapies, immunotherapies or radiation therapy. Compounds described herein may be effective in combination with anti-hormonal agents for treatment of breast cancer and other tumors. Suitable examples are anti-estrogen agents including but not limited to tamoxifen and toremifene, aromatase inhibitors including but not limited to letrozole, anastrozole, and exemestane, adrenocorticosteroids (e.g. prednisone), progestins (e.g. megastrol acetate), and estrogen receptor antagonists (e.g. fulvestrant). Suitable anti-hormone agents used for treatment of prostate and other cancers may also be combined with compounds described herein. These include anti-androgens including but not limited to flutamide, bicalutamide, and nilutamide, luteinizing hormone-releasing hormone (LHRH) analogs including leuprolide, goserelin, triptorelin, and histrelin, LHRH antagonists (e.g. degarelix), androgen receptor blockers (e.g. enzalutamide) and agents that inhibit androgen production (e.g. abiraterone).

The compounds described herein may be combined with or in sequence with other agents against membrane receptor kinases especially for patients who have developed primary or acquired resistance to the targeted therapy. These therapeutic agents include inhibitors or antibodies against EGFR, Her2, VEGFR, c-Met, Ret, IGFR1, or Flt-3 and against cancer-associated fusion protein kinases such as Bcr-Abl and EML4-Alk. Inhibitors against EGFR include gefitinib and erlotinib, and inhibitors against EGFR/Her2 include but are not limited to dacomitinib, afatinib, lapitinib and neratinib. Antibodies against the EGFR include but are not limited to cetuximab, panitumumab and necitumumab. Inhibitors of c-Met may be used in combination with FGFR inhibitors. These include onartumzumab, tivantnib, and INC-280. Agents against Abl (or Bcr-Abl) include imatinib, dasatinib, nilotinib, and ponatinib and those against Alk (or EML4-ALK) include crizotinib.

Angiogenesis inhibitors may be efficacious in some tumors in combination with inhibitors described herein. These include antibodies against VEGF or VEGFR or kinase inhibitors of VEGFR. Antibodies or other therapeutic proteins against VEGF include bevacizumab and aflibercept. Inhibitors of VEGFR kinases and other anti-angiogenesis inhibitors include but are not limited to sunitinib, sorafenib, axitinib, cediranib, pazopanib, regorafenib, brivanib, and vandetanib

Activation of intracellular signaling pathways is frequent in cancer, and agents targeting components of these pathways have been combined with receptor targeting agents to enhance efficacy and reduce resistance. Examples of agents that may be combined with compounds described herein include inhibitors of the PI3K-AKT-mTOR pathway, inhibitors of the Raf-MAPK pathway, inhibitors of JAK-STAT pathway, and inhibitors of protein chaperones and cell cycle progression.

Agents against the PI3 kinase include but are not limited topilaralisib, idelalisib, buparlisib. Inhibitors of mTOR such as rapamycin, sirolimus, temsirolimus, and everolimus may be combined with compounds described herein. Other suitable examples include but are not limited to vemurafenib and dabrafenib (Raf inhibitors) and trametinib, selumetinib and GDC-0973 (MEK inhibitors). Inhibitors of one or more JAKs (e.g., ruxolitinib, baricitinib, tofacitinib), Hsp90 (e.g., tanespimycin), cyclin dependent kinases (e.g., palbociclib), HDACs (e.g., panobinostat), PARP (e.g., olaparib), and proteasomes (e.g., bortezomib, carfilzomib) can also be combined with compounds described herein. In some embodiments, the JAK inhibitor is selective for JAK1 over JAK2 and JAK3.

Other suitable agents for use in combination with compounds described herein include chemotherapy combinations such as platinum-based doublets used in lung cancer and other solid tumors (cisplatin or carboplatin plus gemcitabine; cisplatin or carboplatin plus docetaxel; cisplatin or carboplatin plus paclitaxel; cisplatin or carboplatin plus pemetrexed) or gemcitabine plus paclitaxel bound particles.

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide, ifosfamide, melphalan, chlorambucil, pipobroman, triethylene-melamine, triethylenethiophosphoramine, busulfan, carmustine, lomustine, streptozocin, dacarbazine, and temozolomide.

Other suitable agents for use in combination with compounds described herein include steroids including 17 alpha-ethinylestradiol, diethylstilbestrol, testosterone, prednisone, fluoxymesterone, methylprednisolone, methyltestosterone, prednisolone, triamcinolone, chlorotrianisene, hydroxyprogesterone, aminoglutethimide, and medroxyprogesteroneacetate.

Other suitable agents for use in combination with compounds described herein include: dacarbazine (DTIC), optionally, along with other chemotherapy drugs such as carmustine (BCNU) and cisplatin; the “Dartmouth regimen,” which consists of DTIC, BCNU, cisplatin and tamoxifen; a combination of cisplatin, vinblastine, and DTIC; or temozolomide. Compounds described herein may also be combined with immunotherapy drugs, including cytokines such as interferon alpha, interleukin 2, and tumor necrosis factor (TNF) in.

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (e.g., vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel, mithramycin, deoxycoformycin, mitomycin-C, L-asparaginase, interferons (especially IFN-α), etoposide, and teniposide.

Other cytotoxic agents include navelbene, CPT-11, anastrazole, letrazole, capecitabine, reloxafine, cyclophosphamide, ifosamide, and droloxafine.

Also suitable are cytotoxic agents such as epidophyllotoxin; an antineoplastic enzyme; a topoisomerase inhibitor; procarbazine; mitoxantrone; platinum coordination complexes such as cis-platin and carboplatin; biological response modifiers; growth inhibitors; antihormonal therapeutic agents; leucovorin; tegafur; and haematopoietic growth factors.

Other anti-cancer agent(s) include antibody therapeutics such as trastuzumab (Herceptin), antibodies to costimulatory molecules such as CTLA-4, 4-1BB, PD-L1 and PD-1 antibodies, or antibodies to cytokines (IL-10, TGF-β, etc.).

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines include dendritic cells, synthetic peptides, DNA vaccines and recombinant viruses. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). Non-limiting examples of tumor vaccines that can be used include peptides of melanoma antigens, such as peptides of gp 100, MAGE antigens, Trp-2, MARTI and/or tyrosinase, or tumor cells transfected to express the cytokine GM-CSF.

The compounds of the present disclosure can be used in combination with bone marrow transplant for the treatment of a variety of tumors of hematopoietic origin (see e.g., U.S. Pat. Nos. 9,233,985, 10,065,974, 10,287,303, 8,524,867, the disclosures of which are incorporated by reference herein in their entireties).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

As provided throughout, the additional compounds, inhibitors, agents, etc. can be combined with the present compound in a single or continuous dosage form, or they can be administered simultaneously or sequentially as separate dosage forms.

### Pharmaceutical Formulations and Dosage Forms

When employed as pharmaceuticals, the compounds of the disclosure can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer: intratracheal or intranasal), oral, or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, the compound of the disclosure or a pharmaceutically acceptable salt thereof, in combination with one or more pharmaceutically acceptable carriers (excipients). In some embodiments, the composition is suitable for topical administration. In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form, each dosage containing from about 5 to about 1000 mg (1 g), more usually about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions of the disclosure contain from about 5 to about 50 mg of the active ingredient. One having ordinary skill in the art will appreciate that this embodies compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compositions of the disclosure contain from about 50 to about 500 mg of the active ingredient. One having ordinary skill in the art will appreciate that this embodies compositions containing about 50 to about 100, about 100 to about 150, about 150 to about 200, about 200 to about 250, about 250 to about 300, about 350 to about 400, or about 450 to about 500 mg of the active ingredient.

In some embodiments, the compositions of the disclosure contain from about 500 to about 1000 mg of the active ingredient. One having ordinary skill in the art will appreciate that this embodies compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

Similar dosages may be used of the compounds described herein in the methods and uses of the disclosure.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the disclosure. The topical formulations can be suitably packaged in tubes of, for example, 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the disclosure can further include one or more additional pharmaceutical agents such as a chemotherapeutic, steroid, anti-inflammatory compound, or immunosuppressant, examples of which are listed herein.

### Labeled Compounds and Assay Methods

Another aspect of the present disclosure relates to labeled compounds of the disclosure (radio-labeled, fluorescent-labeled, etc.) that would be useful not only in imaging techniques but also in assays, both in vitro and in vivo, for localizing and quantitating V617F in tissue samples, including human, and for identifying V617F inhibitors by binding of a labeled compound. Substitution of one or more of the atoms of the compounds of the present disclosure can also be useful in generating differentiated ADME (Adsorption, Distribution, Metabolism and Excretion.) Accordingly, the present disclosure includes V617F assays that contain such labeled or substituted compounds.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formula I can be optionally substituted with deuterium atoms, such as —CD₃ (i.e., trideuteromethyl) being substituted for —CH₃). In some embodiments, alkyl groups of the disclosed Formulas (e.g., Formula I) can be perdeuterated.

One or more constituent atoms of the compounds presented herein can be replaced or substituted with isotopes of the atoms in natural or non-natural abundance. In some embodiments, the compound includes at least one deuterium atom. For example, one or more hydrogen atoms in a compound presented herein can be replaced or substituted by deuterium (e.g., one or more hydrogen atoms of a C₁₋₆ alkyl group can be replaced by deuterium atoms, such as —CD₃ being substituted for —CH₃). In some embodiments, the compound includes two or more deuterium atoms. In some embodiments, the compound includes 1-2, 1-3, 1-4, 1-5, 1-6, 1-8, 1-10, 1-12, 1-14, 1-16, 1-18, or 1-20 deuterium atoms. In some embodiments, all of the hydrogen atoms in a compound can be replaced or substituted by deuterium atoms.

In some embodiments, each hydrogen atom of the compounds provided herein, such as hydrogen atoms attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, is optionally replaced by deuterium atoms.

In some embodiments, each hydrogen atom of the compounds provided herein, such as hydrogen atoms to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, is replaced by deuterium atoms (i.e., the alkyl, alkenyl, alkynyl, aryl, phenyl, cycloalkyl, heterocycloalkyl, or heteroaryl substituents, or —C₁₋₄ alkyl-, alkylene, alkenylene, and alkynylene linking groups are perdeuterated).

In some embodiments, 1, 2, 3, 4, 5, 6, 7, 8, 9, 10, 11, or 12 hydrogen atoms, attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, are optionally replaced by deuterium atoms.

In some embodiments, 1, 2, 3, 4, 5, 6, 7, or 8 hydrogen atoms, attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene and alkynylene linking groups, as described herein, are optionally replaced by deuterium atoms.

In some embodiments, the compound provided herein (e.g., the compound of any of Formulas I-IVo), or a pharmaceutically acceptable salt thereof, comprises at least one deuterium atom.

In some embodiments, the compound provided herein (e.g., the compound of any of Formulas I-IVo), or a pharmaceutically acceptable salt thereof, comprises two or more deuterium atoms.

In some embodiments, the compound provided herein (e.g., the compound of any of Formulas I-IVo), or a pharmaceutically acceptable salt thereof, comprises three or more deuterium atoms.

In some embodiments, for a compound provided herein (e.g., the compound of any of Formulas I-IVo), or a pharmaceutically acceptable salt thereof, all of the hydrogen atoms are replaced by deuterium atoms (i.e., the compound is “perdeuterated”).

Synthetic methods for including isotopes into organic compounds are known in the art (Deuterium Labeling in Organic Chemistry by Alan F. Thomas (New York, N.Y., Appleton-Century-Crofts, 1971: The Renaissance of H/D Exchange by Jens Atzrodt, Volker Derdau, Thorsten Fey and Jochen Zimmermann, Angew. Chem. Int. Ed. 2007, 7744-7765: The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro V617F labeling and competition assays, compounds that incorporate ³H, ¹⁴C, ⁸²Br, ¹²⁵I, ¹³¹I or ³⁵S can be useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

A labeled compound of the disclosure can be used in a screening assay to identify/evaluate compounds. For example, a newly synthesized or identified compound (i.e., test compound) which is labeled can be evaluated for its ability to bind V617F by monitoring its concentration variation when contacting with V617F, through tracking of the labeling. For example, a test compound (labeled) can be evaluated for its ability to reduce binding of another compound which is known to bind to V617F (i.e., standard compound). Accordingly, the ability of a test compound to compete with the standard compound for binding to V617F directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Kits

The present disclosure also includes pharmaceutical kits useful, for example, in the treatment or prevention of V617F-associated diseases or disorders as described herein, which include one or more containers containing a pharmaceutical composition comprising a therapeutically effective amount of a compound of the disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### EXAMPLES

Preparatory LC-MS purifications of some of the compounds prepared were performed on Waters mass directed fractionation systems. The basic equipment setup, protocols, and control software for the operation of these systems have been described in detail in the literature (see e.g. “Two-Pump At Column Dilution Configuration for Preparative LC-MS”, K. Blom, J. Combi. Chem., 4, 295 (2002); “Optimizing Preparative LC-MS Configurations and Methods for Parallel Synthesis Purification”, K. Blom, R. Sparks, J. Doughty, G. Everlof, T. Haque, A. Combs, J. Combi. Chem., 5, 670 (2003); and “Preparative LC-MS Purification: Improved Compound Specific Method Optimization”, K. Blom, B. Glass, R. Sparks, A. Combs, J. Combi. Chem., 6, 874-883 (2004)).

The compounds separated were typically subjected to analytical liquid chromatography mass spectrometry (LCMS) for purity analysis under the following conditions: Instrument=Agilent 1100 series, LC/MSD; Column: Waters Sunfire™ C₁₈ 5 μm, 2.1×50 mm, Buffers: mobile phase A: 0.025% TFA in water and mobile phase B: acetonitrile; gradient 2% to 80% B in 3 minutes with flow rate 2.0 mL/minute.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:

pH=2 purifications: Waters Sunfire™ C₁₈ 5 μm, 30×100 mm or Waters XBridge™ C₁₈ 5 μm, 30×100 mm column, eluting with mobile phase A: 0.1% TFA (trifluoroacetic acid) in water and mobile phase B: acetonitrile; the flow rate was 60 mL/minute, the separating gradient was optimized for each compound using the Compound Specific Method Optimization protocol as described in the literature (see e.g., “Preparative LCMS Purification: Improved Compound Specific Method Optimization”, K. Blom, B. Glass, R. Sparks, A. Combs, J. Comb. Chem., 6, 874-883 (2004)).

pH=10 purifications: Waters XBridge™ C₁₈ 5 μm, 30×100 mm column, eluting with mobile phase A: 0.1% NH₄OH in water and mobile phase B: acetonitrile; the flow rate was 60 mL/minute, the separating gradient was optimized for each compound using the Compound Specific Method Optimization protocol as described in the literature (see e.g., “Preparative LCMS Purification: Improved Compound Specific Method Optimization”, K. Blom, B. Glass, R. Sparks, A. Combs, J. Comb. Chem., 6, 874-883 (2004)).

### Intermediate A. Methyl ((1R,3R)-3-(6-chloro-3-methyl-2-oxo-2,3-dihydro-1H-imidazo[4,5-c]pyridin-1-yl)cyclopentyl)carbamate

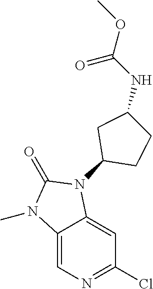 [SMILES: COC(=O)N[C@@H]1CC[C@@H](n2c(=O)n(C)c3cnc(Cl)cc32)C1]

### Step 1. tert-Butyl ((1R,3R)-3-((2-chloro-5-nitropyridin-4-yl)amino)cyclopentyl)carbamate

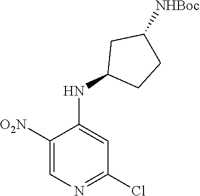 [SMILES: CC(C)(C)OC(=O)N[C@@H]1CC[C@@H](Nc2cc(Cl)ncc2[N+](=O)[O-])C1]

To a vial containing 2,4-dichloro-5-nitropyridine (2.02 g, 10.49 mmol) and DIPEA (2.09 ml, 11.98 mmol) in acetonitrile (6.7 mL) was added tert-butyl ((1R,3R)-3-aminocyclopentyl)carbamate (2 g, 9.99 mmol). The reaction mixture was heated to 60° C. for 16 h and then cooled to r.t. The reaction mixture was diluted with water (100 mL) and filtered. The solid was washed with water (30 mL) for 3 times and dried in vacuo. The obtained crude product was used in the next step without further purification. LCMS calculated for C₁₅H₂₂ClN₄O₄ (M+H)⁺: m/z=357.1; found 357.2.

### Step 2. tert-Butyl ((1R,3R)-3-((5-amino-2-chloropyridin-4-yl)amino)cyclopentyl)carbamate

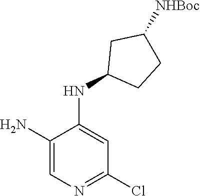 [SMILES: CC(C)(C)OC(=O)N[C@@H]1CC[C@@H](Nc2cc(Cl)ncc2N)C1]

A round-bottom flask containing tert-butyl ((1R,3R)-3-((2-chloro-5-nitropyridin-4-yl)amino)cyclopentyl)carbamate (3.50 g, 9.80 mmol) and ammonium chloride (5.30 g, 99.0 mmol) in MeOH (30 mL) and water (30 mL) was heated to 45° C. Zinc powder (6.50 g, 99.0 mmol) was added in portions (over 10 times) to the reaction mixture with vigorous stirring. After stirring at 45° C. for 1 h, the reaction mixture was filtered and washed with CH₂Cl₂ (30 mL) for three times. The filtrate was collected and extracted with CH₂Cl₂. The organic phase was washed with brine, dried over MgSO₄ and concentrated in vacuo. The obtained crude product was used in the next step without further purification. LCMS calculated for C₁₅H₂₄ClN₄O₂ (M+H)⁺: m/z=327.2; found 327.2.

### Step 3. tert-Butyl ((1R,3R)-3-(6-chloro-2-oxo-2,3-dihydro-1H-imidazo[4,5-c]pyridin-1-yl)cyclopentyl)carbamate

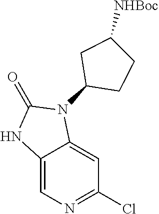 [SMILES: CC(C)(C)OC(=O)N[C@@H]1CC[C@@H](n2c(=O)[nH]c3cnc(Cl)cc32)C1]

To a vial containing tert-butyl ((1R,3R)-3-((5-amino-2-chloropyridin-4-yl)amino)cyclopentyl)carbamate (2.90 g, 8.87 mmol) in acetonitrile (44.4 mL) was added bis(2,5-dioxopyrrolidin-1-yl) carbonate (2.50 g, 9.76 mmol) in portions (over 5 times). After stirring at r.t. for 16 h, the reaction mixture was filtered to obtain the product as off-white solid. The filtrate was concentrated in vacuo and was washed with aqueous saturated sodium bicarbonate solution and extracted with CH₂Cl₂. The organic phase was washed with brine, dried over MgSO₄ and concentrated in vacuo. The obtained mixture was washed with acetonitrile (10.0 ml) and filtered to obtain the second portion of the product as off-white solid. The obtained crude product was used in the next step without further purification. LCMS calculated for C₁₆H₂₂ClN₄O₃ (M+H)⁺: m/z=353.1; found: 353.1.

### Step 4. 1-((1R,3R)-3-Aminocyclopentyl)-6-chloro-3-methyl-1,3-dihydro-2H-imidazo[4,5-c]pyridin-2-one

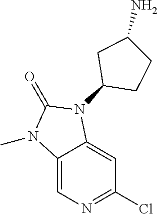 [SMILES: Cn1c(=O)n([C@@H]2CC[C@@H](N)C2)c2cc(Cl)ncc21]

To a vial containing tert-butyl ((1R,3R)-3-(6-chloro-2-oxo-2,3-dihydro-1H-imidazo[4,5-c]pyridin-1-yl)cyclopentyl)carbamate (2.60 g, 7.37 mmol) and cesium carbonate (4.80 g, 14.74 mmol) in THF (24.6 mL) was added iodomethane (2.61 g, 1.15 mL, 18.42 mmol). After stirring at r.t. for 6 h, the reaction mixture was diluted with CH₂Cl₂ (50 mL), filtered through a celite pad and washed with CH₂Cl₂ (15 mL) for 3 times. The filtrate was concentrated in vacuo and was washed with aqueous saturated sodium bicarbonate solution. The organic phase was washed with brine, dried over MgSO₄ and concentrated in vacuo. The crude material was redissolved in CH₂Cl₂ (1.0 ml) and TFA (5.0 ml). After stirring at 30° C. for 1 h, the reaction mixture was concentrated in vacuo. The crude material was redissolved in CH₂Cl₂ (20 mL) and the pH of the mixture was adjusted to ˜10 with ammonia aqueous solution and then extracted into CH₂Cl₂. The organic phase was washed with brine, dried over MgSO₄ and concentrated in vacuo. The obtained crude product was used in the next step without further purification. LCMS calculated for C₁₂H₁₆ClN₄O (M+H)⁺: m/z=267.1; found: 267.1.

### Step 5. Methyl ((1R,3R)-3-(6-chloro-3-methyl-2-oxo-2,3-dihydro-1H-imidazo[4,5-c]pyridin-1-yl)cyclopentyl)carbamate

To a flask containing 1-((1R,3R)-3-aminocyclopentyl)-6-chloro-3-methyl-1,3-dihydro-2H-imidazo[4,5-c]pyridin-2-one (1.80 g, 6.75 mmol) and DIPEA (1.77 ml, 10.12 mmol) in CH₂Cl₂ (45 mL) and EtOH (0.5 mL) at 0° C. was added methyl carbonochloridate (700 mg, 0.58 mL, 7.42 mmol). After stirring at 0° C. for 5 min, the reaction mixture was quenched with MeOH and concentrated in vacuo. The obtained crude product was purified by Biotage Isolera to give the desired product as white solid. LCMS calculated for C₁₄H₁₈ClN₄O₃ (M+H)⁺: m/z=325.1; found: 325.1.

### Example 1. Ethyl 2-((3′-cyano-5-((1-((1R,3R)-3-((methoxycarbonyl)amino)cyclopentyl)-3-methyl-2-oxo-2,3-dihydro-1H-imidazo[4,5-c]pyridin-6-yl)amino)-[1,1′-biphenyl]-3-yl)amino)oxazole-5-carboxylate

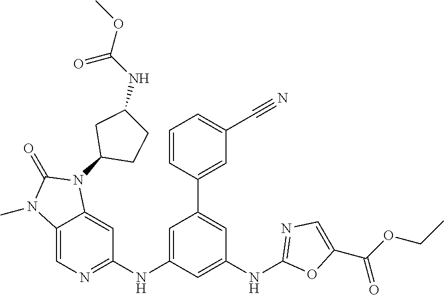 [SMILES: CCOC(=O)c1cnc(Nc2cc(Nc3cc4c(cn3)n(C)c(=O)n4[C@@H]3CC[C@@H](NC(=O)OC)C3)cc(-c3cccc(C#N)c3)c2)o1]

### Step 1. 3′-Amino-5′-nitro-[1.l′-biphenyl]-3-carbonitrile

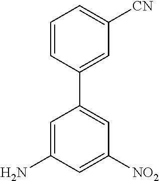 [SMILES: N#Cc1cccc(-c2cc(N)cc([N+](=O)[O-])c2)c1]

To a reaction vial containing 3-bromo-5-nitroaniline (216 mg, 1.0 mmol), (4-cyanophenyl)boronic acid (176 mg, 1.2 mmol), [1,1′-bis(diphenylphosphino)ferrocene]dichloropalladium(II)-dichloromethane complex (82 mg, 0.1 mmol) and potassium carbonate (276 mg, 2 mmol) was added acetonitrile (2 mL) and water (0.4 mL). The reaction mixture was stirred at 85° C. for 1 h. The reaction mixture was allowed to cool to r.t and extracted with EtOAc. The organic layer was combined, dried over sodium sulfate, filtered, and the solvents were evaporated in vacuo. The crude material was purified by Biotage Isolera to give the product as a brown solid. LCMS calculated for C₁₃H₁₀N₃O₂(M+H)⁺: m/z=240.1; found 240.1.

### Step 2. Methyl ((1R,3R)-3-(6-((3′-cyano-5-nitro-[1.l′-biphenyl]-3-yl)amino)-3-methyl-2-oxo-2,3-dihydro-1H-imidazo[4,5-c]pyridin-1-yl)cyclopentyl)carbamate

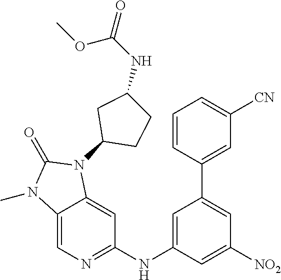 [SMILES: COC(=O)N[C@@H]1CC[C@@H](n2c(=O)n(C)c3cnc(Nc4cc(-c5cccc(C#N)c5)cc([N+](=O)[O-])c4)cc32)C1]

A reaction vial containing methyl ((1R,3R)-3-(6-chloro-3-methyl-2-oxo-2,3-dihydro-1H-imidazo[4,5-c]pyridin-1-yl)cyclopentyl)carbamate (325 mg, 1 mmol), 3′-amino-5′-nitro-[1,1′-biphenyl]-3-carbonitrile (360 mg, 1.5 mmol), tris(dibenzylideneacetone)dipalladium(0) (91 mg, 0.1 mmol), Xantphos (116 mg, 0.2 mmol) and cesium carbonate (652 mg, 2 mmol) was evacuated and backfilled with nitrogen. 1,4-Dioxane (5 mL) was added to the reaction mixture, which was then stirred at 110° C. for 5 h. The reaction mixture was cooled to r.t and diluted with EtOAc. The organic layer was subsequently washed with water and brine, dried over sodium sulfate, filtered, and the solvents were evaporated in vacuo. The crude material was purified by Biotage Isolera. LCMS calculated for C₂₇H₂₆N₇O₅ (M+H)⁺: m/z=528.2; found 528.2.

### Step 3. Methyl ((1R,3R)-3-(6-((5-amino-3′-cyano-[1.l′-biphenyl]-3-yl)amino)-3-methyl-2-oxo-2,3-dihydro-1H-imidazo[4,5-c]pyridin-1-yl)cyclopentyl) carbamate

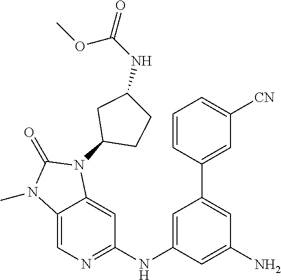 [SMILES: COC(=O)N[C@@H]1CC[C@@H](n2c(=O)n(C)c3cnc(Nc4cc(N)cc(-c5cccc(C#N)c5)c4)cc32)C1]

To a reaction vial containing methyl ((1R,3R)-3-(6-((3′-cyano-5-nitro-[1,1′-bipheny]]-3-yl)amino)-3-methyl-2-oxo-2,3-dihydro-1H-imidazo[4,5-c]pyridin-1-yl)cyclopentyl)carbamate (50 mg, 0.1 mmol) was added MeOH (1 mL) and saturated NH₄Cl (1 mL), followed by Zn (6.5 mg, 1 mmol). The reaction mixture was stirred at 55° C. for 1 h. The reaction mixture was allowed to cool to r.t, basified with saturated NaHCO₃ and extracted with EtOAc. The organic layer was combined, dried over sodium sulfate, filtered, and the solvents were evaporated in vacuo. The crude material was used in the next step without further purification. LCMS calculated for C₂₇H₂₈N₇O₃ (M+H)⁺: m/z=498.2; found 498.2.

### Step 4. Ethyl 2-((3′-cyano-5-((1-((1R,3R)-3-((methoxycarbonyl)amino)cyclopentyl)-3-methyl-2-oxo-2,3-dihydro-1H-imidazo[4,5-c]pyridin-6-yl)amino)-[1,1′-biphenyl]-3-yl)amino)oxazole-5-carboxylate

To a microwave vial containing methyl ((1R,3R)-3-(6-((5-amino-3′-cyano-[1,1′-biphenyl]-3-yl)amino)-3-methyl-2-oxo-2,3-dihydro-1H-imidazo[4,5-c]pyridin-1-yl)cyclopentyl)carbamate (20 mg, 0.04 mmol) and ethyl 2-chlorooxazole-5-carboxylate (14 mg, 0.08 mmol) was added isopropanol (0.4 mL), which was then microwaved at 130° C. for 30 min. The reaction mixture was cooled to r.t, diluted with MeCN and purified by prep-LCMS (XBridge C18 column, eluting with a gradient of acetonitrile/water containing 0.1% TFA, at flow rate of 60 mL/min. LCMS calculated for C₃₃H₃₃N₈O₆ (M+H)⁺: m/z=637.2; found: 637.2. ¹H NMR (400 MHZ, DMSO-d₆) δ 11.02 (s, 1H), 9.49 (s, 1H), 8.01 (s, 2H), 7.93 (d, J=8.0 Hz, 1H), 7.88 (d, J=7.6 Hz, 1H), 7.87 (s, 1H), 7.80 (s, 1H), 7.73 (t, J=7.8 Hz, 1H), 7.65 (s, 1H), 7.52 (s, 1H), 7.34 (d, J=7.0 Hz, 1H), 6.87 (s, 1H), 4.93 (p, J=8.7 Hz, 1H), 4.29 (q, J=7.1 Hz, 2H), 4.17 (s, 1H), 3.53 (s, 3H), 3.34 (s, 3H), 2.27 (dd, J=14.2, 7.4 Hz, 1H), 2.19-1.96 (m, 1H), 1.89 (td, J=11.6, 5.8 Hz, 1H), 1.69-1.50 (m, 1H), 1.30 (t, J=7.1 Hz, 3H).

### Example 2. Methyl ((1R,3R)-3-(6-((3′-cyano-5-(4-fluorotetrahydro-2H-pyran-4-carboxamido)-[1,1′-biphenyl]-3-yl)amino)-3-methyl-2-oxo-2,3-dihydro-1H-imidazo[4,5-c]pyridin-1-yl)cyclopentyl)carbamate

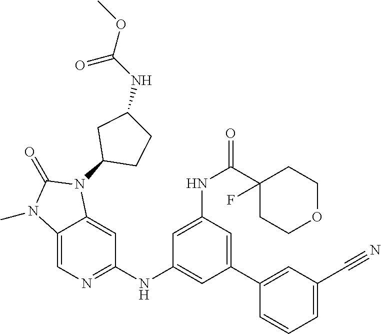 [SMILES: COC(=O)N[C@@H]1CC[C@@H](n2c(=O)n(C)c3cnc(Nc4cc(NC(=O)C5(F)CCOCC5)cc(-c5cccc(C#N)c5)c4)cc32)C1]

To a reaction vial containing methyl ((1R,3R)-3-(6-((5-amino-3′-cyano-[1,1′-biphenyl]-3-yl)amino)-3-methyl-2-oxo-2,3-dihydro-1H-imidazo[4,5-c]pyridin-1-yl)cyclopentyl)carbamate (10 mg, 0.02 mmol), 4-fluorotetrahydro-2H-pyran-4-carboxylic acid (6 mg, 0.04 mmol), HATU (9 mg, 0.024 mmol) and DIPEA (7 μL, 0.04 mmol) was added DMF (0.4 mL). The reaction mixture was stirred at r.t for 1 h. The reaction mixture was diluted with MeCN and purified by prep-LCMS (XBridge C18 column, eluting with a gradient of acetonitrile/water containing 0.1% TFA, at flow rate of 60 mL/min. LCMS calculated for C₃₃H₃₅FN₇O₅ (M+H)⁺: m/z=628.3; found: 628.3. ¹H NMR (400 MHZ, DMSO-d₆) δ 10.13 (s, 1H), 9.47 (s, 1H), 8.03 (d, J=6.8 Hz, 2H), 8.01 (s, 1H), 7.96 (d, J=8.0 Hz, 1H), 7.87 (d, J=7.7 Hz, 1H), 7.77-7.49 (m, 3H), 7.34 (d, J=7.0 Hz, 1H), 6.87 (s, 1H), 4.93 (p, J=8.7 Hz, 1H), 4.38-4.04 (m, 1H), 3.89 (dd, J=11.3, 5.0 Hz, 2H), 3.64 (dd, J=12.8, 10.4 Hz, 2H), 3.54 (s, 3H), 3.34 (s, 3H), 2.36-1.79 (m, 9H), 1.61 (dt, J=12.9, 7.3 Hz, 1H).

### Example 3. Methyl ((1R,3R)-3-(3-(methyl-d₃)-6-((4-(1-methyl-1,6-diazaspiro[3.3]heptane-6-carbonyl)-8-(trifluoromethyl)quinolin-2-yl)amino)-2-oxo-2,3-dihydro-1H-imidazo[4,5-c]pyridin-1-yl)cyclopentyl)carbamate

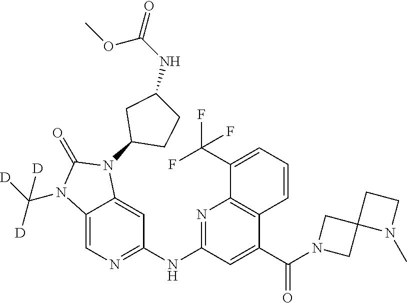 [SMILES: [2H]C([2H])([2H])n1c(=O)n([C@@H]2CC[C@@H](NC(=O)OC)C2)c2cc(Nc3cc(C(=O)N4CC5(CCN5C)C4)c4cccc(C(F)(F)F)c4n3)ncc21]

### Step 1. 2-Oxo-8-(trifluoromethyl)-1,2-dihydroquinoline-4-carboxylic acid

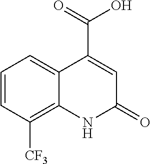 [SMILES: O=C(O)c1cc(=O)[nH]c2c(C(F)(F)F)cccc12]

To a reaction vial containing 7-(trifluoromethyl)indoline-2,3-dione (1.0 g, 4.6 mmol), malonic acid (1.5 g, 14.0 mmol) was added acetic acid (10 mL). The reaction mixture was stirred at 110° C. for 40 h. The reaction mixture was allowed to cool to r.t and water was added. The solid was collected by filtration and was washed with water. The crude material was used in the next step without further purification. LCMS calculated for C₁₁H₇F₃NO₃ (M+H)⁺: m/z=258.0; found 258.0.

### Step 2. Ethyl 2-oxo-8-(trifluoromethyl)-1,2-dihydroquinoline-4-carboxylate

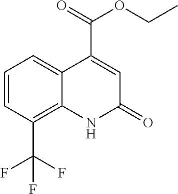 [SMILES: CCOC(=O)c1cc(=O)[nH]c2c(C(F)(F)F)cccc12]

To a suspension of 2-oxo-8-(trifluoromethyl)-1,2-dihydroquinoline-4-carboxylic acid (0.5 g, 2 mmol) in EtOH (10 mL), in an ice bath, was added thionyl chloride (3 mL) dropwise. The mixture was stirred at r.t for 15 min then refluxed for 2 h. The reaction mixture was cooled to r.t and the solvent was evaporated in vacuo. The crude material was used in the next step without further purification. LCMS calculated for C₁₃H₁₁F₃NO₃ (M+H)⁺: m/z=286.1; found 286.1.

### Step 3. Ethyl 2-chloro-8-(trifluoromethyl)quinoline-4-carboxylate

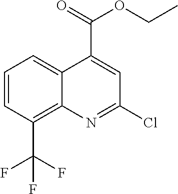 [SMILES: CCOC(=O)c1cc(Cl)nc2c(C(F)(F)F)cccc12]

A mixture of ethyl 2-oxo-8-(trifluoromethyl)-1,2-dihydroquinoline-4-carboxylate (570 mg, 2 mmol) and phosphoryl trichloride (6 mL) was stirred at 100° C. for 30 min. The reaction mixture was allowed to cool to r.t and concentrated in vacuo. The residue was quenched with ice water. The solid was then filtered and washed with water. The crude material was used in the next step without further purification. LCMS calculated for C₁₃H₁₀ClF₃NO₂ (M+H)⁺: m/z=304.0; found 304.0.

### Step 4. Ethyl 2-amino-8-(trifluoromethyl) quinoline-4-carboxylate

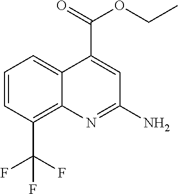 [SMILES: CCOC(=O)c1cc(N)nc2c(C(F)(F)F)cccc12]

To a vial containing ethyl 2-chloro-8-(trifluoromethyl)quinoline-4-carboxylate (607 mg, 2 mmol) (2,4-dimethoxyphenyl)methanamine (669 mg, 4 mmol) in DMSO (5 mL) was added triethylamine (0.7 mL, 5 mmol), which was then stirred at 110° C. for 1 h. The reaction mixture was cooled to r.t. and water was added. The solid was filtered, which was then dissolved in TFA (5 mL) and stirred at 100° C. for 10 min. The mixture was cooled to r.t and concentrated in vacuo and azeotrope with MeCN. The crude material was used in the next step without further purification. LCMS calculated for C₁₃H₁₂F₃N₂O₂ (M+H)⁺: m/z=285.1; found 285.1.

### Step 5. Ethyl 2-((1-((1R,3R)-3-((methoxycarbonyl)amino)cyclopentyl)-3-(methyl-d₃)-2-oxo-2,3-dihydro-1H-imidazo[4,5-c]pyridin-6-yl)amino)-8-(trifluoromethyl)quinoline-4-carboxylate

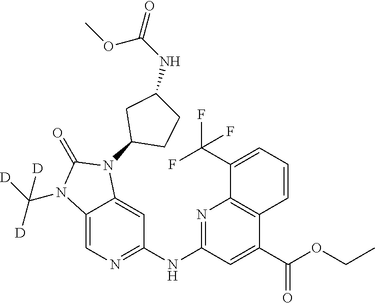 [SMILES: [2H]C([2H])([2H])n1c(=O)n([C@@H]2CC[C@@H](NC(=O)OC)C2)c2cc(Nc3cc(C(=O)OCC)c4cccc(C(F)(F)F)c4n3)ncc21]

A reaction vial containing methyl ((1R,3R)-3-(6-chloro-3-(methyl-d3)-2-oxo-2,3-dihydro-1H-imidazo[4,5-c]pyridin-1-yl)cyclopentyl)carbamate (656 mg, 2 mmol), ethyl 2-amino-8-(trifluoromethyl)quinoline-4-carboxylate (625 mg, 2.2 mmol), xantphos-Pd-G3 (189 mg, 0.2 mmol) and cesium carbonate (1.9 g, 6 mmol) was evacuated and backfilled with nitrogen. 1,4-Dioxane (15 mL) was added to the reaction mixture, which was then stirred at 110° C. for 3 h. The reaction mixture was cooled to r.t and diluted with EtOAc. The organic layer was subsequently washed with water and brine, dried over sodium sulfate, filtered, and the solvents were evaporated in vacuo. The crude material was purified by Biotage Isolera. LCMS calculated for C₂₇H₂₅D₃F₃N₆O₅ (M+H)⁺: m/z=576.2; found 576.2.

### Step 6. 2-((1-((1R,3R)-3-((Methoxycarbonyl)amino)cyclopentyl)-3-(methyl-d₃)-2-oxo-2,3-dihydro-1H-imidazo[4,5-c]pyridin-6-yl)amino)-8-(trifluoromethyl)quinoline-4-carboxylic acid

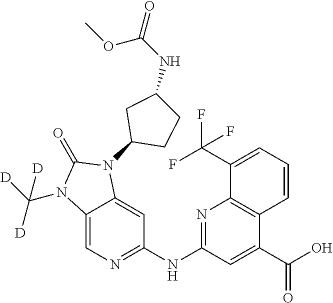 [SMILES: [2H]C([2H])([2H])n1c(=O)n([C@@H]2CC[C@@H](NC(=O)OC)C2)c2cc(Nc3cc(C(=O)O)c4cccc(C(F)(F)F)c4n3)ncc21]

To a reaction vial containing ethyl 2-((1-((1R,3R)-3-((methoxycarbonyl)amino)cyclopentyl)-3-(methyl-d3)-2-oxo-2,3-dihydro-1H-imidazo[4,5-c]pyridin-6-yl)amino)-8-(trifluoromethyl)quinoline-4-carboxylate (800 mg, 1.4 mmol) in THF (10 mL) was added aqueous solution of LiOH (133 mg, 5.6 mmol) and stirred at r.t for 1 h. The mixture was concentrated in vacuo, then the solid was collected by filtration and washed with water. The crude material was used in the next step without further purification. LCMS calculated for C₂₅H₂₁D₃F₃N₆O₅ (M+H)⁺: m/z=548.2; found 548.2.

### Step 7. Methyl ((1R,3R)-3-(3-(methyl-d₃)-6-((4-(1-methyl-1,6-diazaspiro[3.3]heptane-6-carbonyl)-8-(trifluoromethyl)quinolin-2-yl)amino)-2-oxo-2,3-dihydro-1H-imidazo[4,5-c]pyridin-1-yl)cyclopentyl)carbamate

To a reaction vial containing 2-((1-((1R,3R)-3-((methoxycarbonyl)amino)cyclopentyl)-3-(methyl-d3)-2-oxo-2,3-dihydro-1H-imidazo[4,5-c]pyridin-6-yl)amino)-8-(trifluoromethyl)quinoline-4-carboxylic acid (8 mg, 0.015 mmol), HATU (11 mg, 0.029 mg) in DMF (0.5 mL) was added DIPEA (6 mg, 0.044 mmol), followed by 1-methyl-1,6-diazaspiro[3.3]heptane (5 mg, 0.044 mmol). The mixture was stirred at 60° C. for 15 min. The reaction mixture was allowed to cool to r.t, diluted with MeCN and purified by prep-LCMS (XBridge C18 column, eluting with a gradient of acetonitrile/water containing 0.1% TFA, at flow rate of 60 mL/min. LCMS calculated for C₃₁H₃₁D₃F₃N₈O₄ (M+H)⁺: m/z=642.3; found 642.3.

### Example 4. Methyl ((1R,3R)-3-(6-((6-(4-(hydroxymethyl)-3-methylphenyl)pyridin-2-yl)amino)-3-methyl-2-oxo-2,3-dihydro-1H-imidazo[4,5-c]pyridin-1-yl)cyclopentyl)carbamate

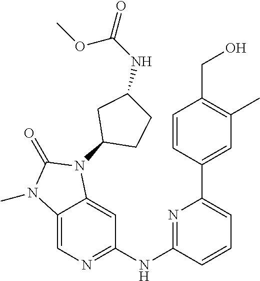

### Step 1. Methyl ((1R,3R)-3-(6-((6-chloropyridin-2-yl)amino)-3-methyl-2-oxo-2,3-dihydro-1H-imidazo[4,5-c]pyridin-1-yl)cyclopentyl) carbamate

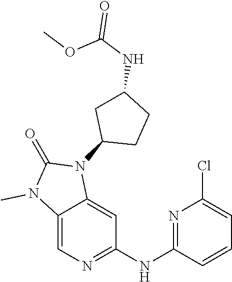 [SMILES: COC(=O)N[C@@H]1CC[C@@H](n2c(=O)n(C)c3cnc(Nc4cccc(Cl)n4)cc32)C1]

The title compound was prepared according to the procedures described in Example 1, with 6-chloropyridin-2-amine replacing 3′-amino-5′-nitro-[1, l′-biphenyl]-3-carbonitrile in Step 2. LCMS calculated for C₁₉H₂₂ClN₆O₃ (M+H)⁺: m/z=417.1; found 417.1.

### Step 2. Methyl ((1R,3R)-3-(6-((6-(4-(hydroxymethyl)-3-methylphenyl)pyridin-2-yl)amino)-3-methyl-2-oxo-2,3-dihydro-1H-imidazo[4,5-c]pyridin-1-yl)cyclopentyl)carbamate

A reaction vial containing methyl ((1R,3R)-3-(6-((6-chloropyridin-2-yl)amino)-3-methyl-2-oxo-2,3-dihydro-1H-imidazo[4,5-c]pyridin-1-yl)cyclopentyl)carbamate (10 mg, 0.024 mmol), (4-(hydroxymethyl)-3-methylphenyl)boronic acid (6 mg, 0.036 mmol), XPhos-Pd-G2 (2 mg, 0.002 mmol) and potassium phosphate tribasic (13 mg, 0.06 mmol) was evacuated and backfilled with nitrogen. 1,4-Dioxane (0.4 mL) and water (0.1 mL) was added to the reaction mixture, which was then stirred at 110° C. for 30 min. The reaction mixture was allowed to cool to r.t, diluted with MeCN and purified by prep-LCMS (XBridge C18 column, eluting with a gradient of acetonitrile/water containing 0.1% TFA, at flow rate of 60 mL/min. LCMS calculated for C₂₇H₃₁N₆O₄ (M+H)⁺: m/z=503.2; found 503.2. ¹H NMR (400 MHz, DMSO-d₆) δ 10.94 (s, 1H), 8.32 (s, 1H), 7.95 (s, 1H), 7.85 (d, J=8.0 Hz, 1H), 7.81 (s, 1H), 7.60 (t, J=8.3 Hz, 2H), 7.44-7.16 (m, 3H), 4.94 (p, J=8.6 Hz, 1H), 4.59 (s, 2H), 4.17 (s, 1H), 3.55 (s, 3H), 3.38 (s, 3H), 2.37 (s, 3H), 2.33-2.26 (m, 1H), 2.19-1.85 (m, 4H), 1.60 (s, 1H).

### Example 5. Methyl ((1R,3R)-3-(6-((6′-methoxy-[2,3′-bipyridin]-6-yl)amino)-3-methyl-2-oxo-2,3-dihydro-1H-imidazo[4,5-c]pyridin-1-yl)cyclopentyl)carbamate

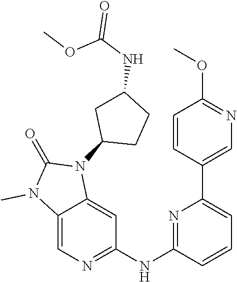 [SMILES: COC(=O)N[C@@H]1CC[C@@H](n2c(=O)n(C)c3cnc(Nc4cccc(-c5ccc(OC)nc5)n4)cc32)C1]

The title compound was prepared according to the procedures described in Example 4, with (6-methoxypyridin-3-yl)boronic acid replacing (4-(hydroxymethyl)-3-methylphenyl)boronic acid in Step 2. LCMS calculated for C₂₅H₂₈N₇O₄ (M+H)⁺: m/z=490.2; found 490.2. ¹H NMR (400 MHZ, DMSO-d₆) δ 10.89 (s, 1H), 8.88 (d, J=2.5 Hz, 1H), 8.34 (dd, J=8.7, 2.6 Hz, 1H), 8.28 (s, 1H), 7.90 (s, 1H), 7.57 (d, J=7.8 Hz, 1H), 7.36 (s, 1H), 7.23 (s, 1H), 6.97 (d, J=8.7 Hz, 1H), 4.94 (p, J=8.8 Hz, 1H), 4.18 (s, 1H), 3.95 (s, 3H), 3.54 (s, 3H), 3.38 (s, 3H), 2.42-2.24 (m, 1H), 2.19-1.82 (m, 4H), 1.66-1.59 (m, 1H).

### Example 6. Methyl ((1R,3R)-3-(6-((4-(4-cyanopiperidine-1-carbonyl)-8-(trifluoromethyl)quinolin-2-yl)amino)-3-(methyl-d3)-2-oxo-2,3-dihydro-1H-imidazo[4,5-c]pyridin-1-yl)cyclopentyl)carbamate

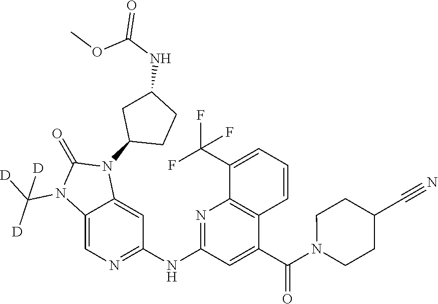 [SMILES: [2H]C([2H])([2H])n1c(=O)n([C@@H]2CC[C@@H](NC(=O)OC)C2)c2cc(Nc3cc(C(=O)N4CCC(C#N)CC4)c4cccc(C(F)(F)F)c4n3)ncc21]

The title compound was prepared according to the procedures described in Example 3, with piperidine-4-carbonitrile replacing 1-methyl-1,6-diazaspiro[3.3]heptane in Step 7. LCMS calculated for C₃₁H₂₉D₃F₃N₈O₄ (M+H)⁺: m/z=640.3; found 640.3.

### Example 7. Methyl ((1R,3R)-3-(3-methyl-6-((6-(3-methyl-1H-indazol-5-yl)pyridin-2-yl)amino)-2-oxo-2,3-dihydro-1H-imidazo[4,5-c]pyridin-1-yl)cyclopentyl)carbamate

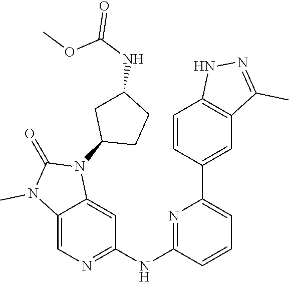

The title compound was prepared according to the procedures described in Example 4, with (3-methyl-1H-indazol-5-yl)boronic acid replacing (4-(hydroxymethyl)-3-methylphenyl)boronic acid in Step 2. LCMS calculated for C₂₇H₂₉N₈O₃ (M+H)⁺: m/z=513.2; found 513.2. ¹H NMR (400 MHZ, DMSO-d₆) δ 12.86 (s, 1H), 11.05 (s, 1H), 8.40 (s, 1H), 8.34 (s, 1H), 8.03 (d, J=8.9 Hz, 1H), 7.97 (t, J=8.3 Hz, 1H), 7.70 (t, J=7.3 Hz, 1H), 7.64 (d, J=8.7 Hz, 1H), 7.40-7.33 (m, 1H), 7.24 (s, 2H), 4.95 (p, J=8.6 Hz, 1H), 4.17 (s, 1H), 3.54 (s, 3H), 3.38 (s, 3H), 2.60 (s, 3H), 2.36-2.22 (m, 1H), 2.19-1.84 (m, 4H), 1.59 (s, 1H).

### Example 8. Methyl ((1R,3R)-3-(6-((6-(2,3-dihydrobenzo[b][1,4]dioxin-6-yl)pyridin-2-yl)amino)-3-methyl-2-oxo-2,3-dihydro-1H-imidazo[4,5-c]pyridin-1-yl)cyclopentyl)carbamate

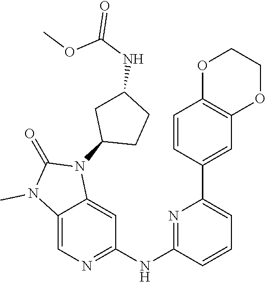 [SMILES: COC(=O)N[C@@H]1CC[C@@H](n2c(=O)n(C)c3cnc(Nc4cccc(-c5ccc6c(c5)OCCO6)n4)cc32)C1]

The title compound was prepared according to the procedures described in Example 4, with (2,3-dihydrobenzo[b][1,4]dioxin-6-yl)boronic acid replacing (4-(hydroxymethyl)-3-methylphenyl)boronic acid in Step 2. LCMS calculated for C₂₇H₂₉N₆O₅ (M+H)⁺: m/z=517.2; found 517.2. ¹H NMR (400 MHZ, DMSO-d₆) δ 10.89 (s, 1H), 8.28 (s, 1H), 7.89 (t, J=7.9 Hz, 1H), 7.56 (s, 1H), 7.53 (d, J=8.1 Hz, 2H), 7.43-7.30 (m, 2H), 7.16 (d, J=8.2 Hz, 1H), 7.03 (d, J=8.4 Hz, 1H), 4.93 (p, J=8.5 Hz, 1H), 4.32 (s, 4H), 4.26-4.03 (m, 1H), 3.54 (s, 3H), 3.38 (s, 3H), 2.29 (dt, J=14.8, 7.8 Hz, 1H), 2.19-1.77 (m, 4H), 1.74-1.52 (m, 1H).

### Example 9. Methyl ((1R,3R)-3-(3-methyl-2-oxo-6-((6-(quinolin-6-yl)pyridin-2-yl)amino)-2,3-dihydro-1H-imidazo[4,5-c]pyridin-1-yl)cyclopentyl)carbamate

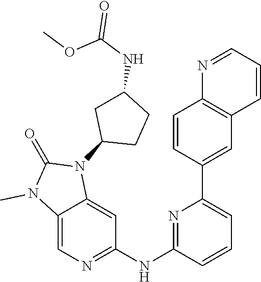 [SMILES: COC(=O)N[C@@H]1CC[C@@H](n2c(=O)n(C)c3cnc(Nc4cccc(-c5ccc6ncccc6c5)n4)cc32)C1]

The title compound was prepared according to the procedures described in Example 4, with quinolin-6-ylboronic acid replacing (4-(hydroxymethyl)-3-methylphenyl)boronic acid in Step 2. LCMS calculated for C₂₈H₂₈N₇O₃ (M+H)⁺: m/z=510.2; found 510.2. ¹H NMR (400 MHz, DMSO-d₆) δ 11.18 (s, 1H), 9.03 (d, J=3.7 Hz, 1H), 8.72 (d, J=2.1 Hz, 1H), 8.64 (d, J=8.3 Hz, 1H), 8.48 (dd, J=8.8, 2.1 Hz, 1H), 8.39 (s, 1H), 8.21 (d, J=8.8 Hz, 1H), 8.04 (t, J=7.9 Hz, 1H), 7.84 (d, J=7.6 Hz, 1H), 7.70 (dd, J=8.3, 4.3 Hz, 1H), 7.36 (s, 2H), 7.27 (d, J=8.3 Hz, 1H), 4.95 (p, J=8.5 Hz, 1H), 4.15 (s, 1H), 3.53 (s, 3H), 3.40 (s, 3H), 2.30 (dt, J=14.7, 7.9 Hz, 1H), 2.20-1.83 (m, 4H), 1.68-1.41 (m, 1H).

### Example 10. Methyl ((1R,3R)-3-(6-((4-(1-hydroxy-3-azabicyclo[3.1.0]hexane-3-carbonyl)-8-(trifluoromethyl)quinolin-2-yl)amino)-3-(methyl-d3)-2-oxo-2,3-dihydro-1H-imidazo[4,5-c]pyridin-1-yl)cyclopentyl)carbamate

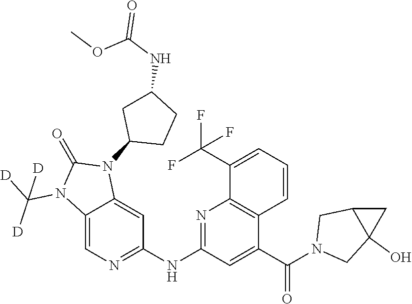 [SMILES: [2H]C([2H])([2H])n1c(=O)n([C@@H]2CC[C@@H](NC(=O)OC)C2)c2cc(Nc3cc(C(=O)N4CC5CC5(O)C4)c4cccc(C(F)(F)F)c4n3)ncc21]

The title compound was prepared according to the procedures described in Example 3, with 3-azabicyclo[3.1.0]hexan-1-ol replacing 1-methyl-1,6-diazaspiro[3.3]heptane in Step 7. LCMS calculated for C₃₀H₂₈D₃F₃N₇O₅ (M+H)⁺: m/z=629.3; found 629.3. ¹H NMR (400 MHz, DMSO) δ 10.88 (s, 1H), 8.29 (d, J=4.3 Hz, 1H), 8.15 (t, J=6.7 Hz, 1H), 8.1 (s, 1H) 7.91 (t, J=6.9 Hz, 1H), 7.57 (q, J=7.2 Hz, 1H), 7.36 (d, J=11.5 Hz, 2H), 7.32 (s, 1H), 4.87 (s, 1H), 4.26 (q, J=7.0 Hz, 1H), 3.80 (d, J=11.9 Hz, 1H), 3.59 (s, 1H), 3.56 (s, 3H), 3.30 (d, J=10.2 Hz, 1H), 2.45-2.29 (m, 1H), 2.27-2.05 (m, 3H), 1.94 (s, 1H), 1.69-1.56 (m, 2H), 1.48 (m, 1H), 1.16-1.00 (m, 1H), 0.56 (q, J=4.3 Hz, 1H). 19F NMR (376 MHz, DMSO) δ −60.2.

### Example 11. Methyl ((1R,3R)-3-(3-methyl-6-((6-(4-methyl-3,4-dihydro-2H-benzo[b][1,4]oxazin-7-yl)pyridin-2-yl)amino)-2-oxo-2,3-dihydro-1H-imidazo[4,5-c]pyridin-1-yl)cyclopentyl)carbamate

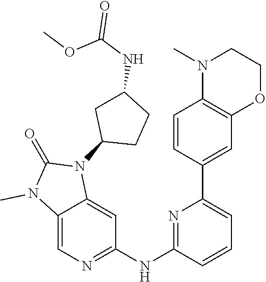 [SMILES: COC(=O)N[C@@H]1CC[C@@H](n2c(=O)n(C)c3cnc(Nc4cccc(-c5ccc6c(c5)OCCN6C)n4)cc32)C1]

The title compound was prepared according to the procedures described in Example 4, with 4-methyl-7-(4,4,5,5-tetramethyl-1,3,2-dioxaborolan-2-yl)-3,4-dihydro-2H-benzo[b][1,4]oxazine replacing (4-(hydroxymethyl)-3-methylphenyl)boronic acid in Step 2. LCMS calculated for C₂₈H₃₂N₇O₄ (M+H)⁺: m/z=530.2; found 530.2. ¹H NMR (400 MHZ, DMSO-d₆) δ 11.13 (s, 1H), 8.31 (s, 1H), 7.92 (s, 1H), 7.51 (dd, J=8.5, 2.2 Hz, 2H), 7.38 (d, J=2.2 Hz, 2H), 7.25 (s, 1H), 7.08 (d, J=8.3 Hz, 1H), 6.87 (d, J=8.5 Hz, 1H), 4.94 (p, J=8.5 Hz, 1H), 4.29 (t, J=4.4 Hz, 2H), 4.21 (d, J=6.7 Hz, 1H), 3.55 (s, 3H), 3.40 (s, 3H), 3.37 (t, J=4.4 Hz, 2H), 2.96 (s, 3H), 2.29 (dt, J=14.8, 7.9 Hz, 1H), 2.20-1.86 (m, 4H), 1.69-1.53 (m, 1H).

### Example 12. Methyl ((1R,3R)-3-(6-((4-(2-oxa-5-azabicyclo[2.2.1]heptane-5-carbonyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

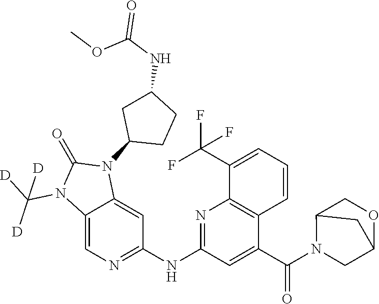 [SMILES: [2H]C([2H])([2H])n1c(=O)n([C@@H]2CC[C@@H](NC(=O)OC)C2)c2cc(Nc3cc(C(=O)N4CC5CC4CO5)c4cccc(C(F)(F)F)c4n3)ncc21]

The title compound was prepared according to the procedures described in Example 3, with 2-oxa-5-azabicyclo[2.2.1]heptane replacing 1-methyl-1,6-diazaspiro[3.3]heptane in Step 7. LCMS calculated for C₃₀H₂₈D₃F₃N₇O₅ (M+H)⁺: m/z=629.3; found 629.3. ¹H NMR (400 MHz, DMSO) δ 11.32 (s, 1H), 8.41 (s, 1H), 8.21 (d, J=7.3 Hz, 1H), 8.07 (dd, J=14.2, 8.1 Hz, 1H), 7.80 (s, 1H), 7.64 (s, 1H), 7.42 (d, J=9.3 Hz, 1H), 7.36 (s, 1H), 4.92 (p, J=8.7 Hz, 1H), 4.76 (d, J=2.5 Hz, 1H), 4.59 (d, J=2.3 Hz, 1H), 4.34-4.18 (m, 1H), 4.00 (d, J=7.6 Hz, 1H), 3.87 (d, J=7.4 Hz, 1H), 3.60 (d, J=7.6 Hz, 1H), 3.55 (s, 3H), 3.21 (d, J=10.0 Hz, 1H), 2.37 (dt, J=14.1, 7.4 Hz, 1H), 2.27-1.88 (m, 5H), 1.76 (d, J=9.8 Hz, 1H), 1.64 (q, J=9.0 Hz, 1H). 19F NMR (376 MHz, DMSO) δ −60.2.

### Example 13. Methyl ((1R,3R)-3-(6-((4-(2-oxa-5-azabicyclo[4.1.0]heptane-5-carbonyl)-8-(trifluoromethyl)quinolin-2-yl)amino)-3-(methyl-d3)-2-oxo-2,3-dihydro-1H-imidazo[4,5-c]pyridin-1-yl)cyclopentyl)carbamate

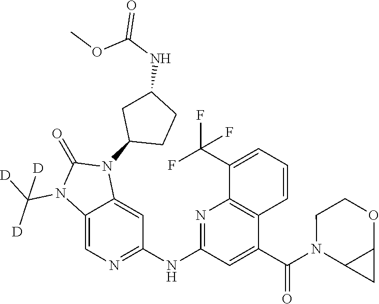 [SMILES: [2H]C([2H])([2H])n1c(=O)n([C@@H]2CC[C@@H](NC(=O)OC)C2)c2cc(Nc3cc(C(=O)N4CCOC5CC54)c4cccc(C(F)(F)F)c4n3)ncc21]

The title compound was prepared according to the procedures described in Example 3, with 2-oxa-5-azabicyclo[4.1.0]heptane replacing 1-methyl-1,6-diazaspiro[3.3]heptane in Step 7. LCMS calculated for C₃₀H₂₈D₃F₃N₇O₅ (M+H)⁺: m/z=629.3; found 629.3. ¹H NMR (400 MHZ, DMSO) δ 11.16 (s, 1H), 8.36 (s, 1H), 8.19 (s, 1H), 8.04 (d, J=8.2 Hz, 1H), 7.93 (s, 1H), 7.62 (s, 1H), 7.49 (s, 1H), 7.37 (d, J=13.8 Hz, 1H), 4.90 (s, 1H), 4.26 (q, J=6.9 Hz, 1H), 4.00-3.81 (m, 1H), 3.79-3.57 (m, 3H), 3.55 (s, 3H), 2.48-2.31 (m, 2H), 2.28-1.83 (m, 4H), 1.73-1.52 (m, 1H), 1.12-0.96 (m, 1H), 0.77 (s, 1H), 0.53 (t, J=6.7 Hz, 1H). 19F NMR (376 MHz, DMSO) δ −60.2.

### Example 14. Methyl (3-((1-((1R,3R)-3-((methoxycarbonyl)amino)cyclopentyl)-3-methyl-2-oxo-2,3-dihydro-1H-imidazo[4,5-c]pyridin-6-yl)amino)-5-(1-methyl-1H-1,2,3-triazol-4-yl)phenyl)carbamate

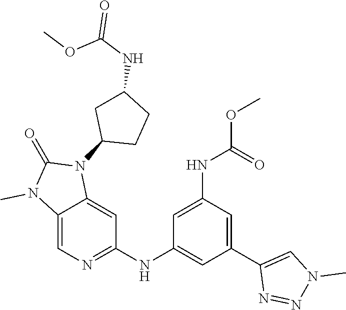

### Step 1. Methyl (3-bromo-5-nitrophenyl) carbamate

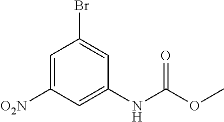

To a mixture of 3-bromo-5-nitroaniline (3.00 g, 13.82 mmol) and N-ethyl-N-isopropylpropan-2-amine (2.95 ml, 16.59 mmol) in THF (8 ml) at r.t. was added methyl carbonochloridate (1.12 ml, 14.51 mmol) dropwise. The mixture was stirred at r.t. for 1 h, then quenched with water. The organic layer was extracted with EtOAc, washed with brine, dried and concentrated. The residue was taken up in DCM (3 ml) and hexane (20 ml) and the resulting mixture was stirred for 0.5 h. The precipitated solid was filtered, washed with hexane and air-dried to give the desired product (2.82 g, 10.25 mmol, 74% yield).

### Step 2. Methyl (3-amino-5-bromophenyl)carbamate

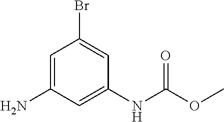

To a solution of methyl (3-bromo-5-nitrophenyl)carbamate (2.82 g, 10.25 mmol) in MeOH (10 ml), THF (10 ml) and water (10 ml) was added ammonium chloride (4.39 g, 82 mmol) and zinc (4.02 g, 61.5 mmol) and stirred at 60° C. for 1 h. The reaction mixture was then cooled to r.t., filtered and rinsed with EtOAc. The obtained filtrate was washed with brine, dried and concentrated. The crude product was taken up in DCM (20 ml) and hexane (20 ml) and stirred for 1 h. The precipitated solid was filtered, washed with hexane and dried to give the desired product (2.31 g, 9.43 mmol, 92% yield). LCMS calculated for C₈H₁₀BrN₂O₂ (M+H)⁺: m/z=245.0; found 245.0.

### Step 3. Methyl (3-amino-5-(1-methyl-1H-1,2,3-triazol-4-yl)phenyl)carbamate

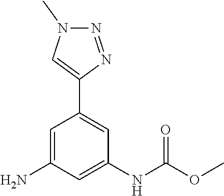 [SMILES: COC(=O)Nc1cc(N)cc(-c2cn(C)nn2)c1]

In a sealed vial, a mixture of methyl (3-amino-5-bromophenyl)carbamate (12 mg, 0.049 mmol), 1-methyl-4-(4,4,5,5-tetramethyl-1,3,2-dioxaborolan-2-yl)-1H-1,2,3-triazole (15 mg, 0.073 mmol), chloro(2-dicyclohexylphosphino-2′,4′,6′-tri-i-propyl-1, l′-biphenyl)(2′-amino-1,1′-biphenyl-2-yl) palladium(II) (4 mg, 4.90 μmol) and potassium phosphate tribasic (21 mg, 0.098 mmol) in 1,4-dioxane (1 ml) and water (0.2 ml) was degassed and then stirred at 80° C. for 1 h. The mixture was then cooled to r.t., and concentrated. The obtained residue was purified by Biotage Isolera to obtain desired product. LCMS calculated for C₁₁H₁₄N₅O₂ (M+H)⁺: m/z=248.1; found 248.1.

### Step 4. Methyl (3-((1-((1R,3R)-3-((methoxycarbonyl)amino)cyclopentyl)-3-methyl-2-oxo-2,3-dihydro-1H-imidazo[4,5-c]pyridin-6-yl)amino)-5-(1-methyl-1H-1,2,3-triazol-4-yl)phenyl)carbamate

A mixture of methyl ((1R,3R)-3-(6-chloro-3-methyl-2-oxo-2,3-dihydro-1H-imidazo[4,5-c]pyridin-1-yl)cyclopentyl)carbamate (8 mg, 0.025 mmol), methyl (3-amino-5-(1-methyl-1H-1,2,3-triazol-4-yl)phenyl)carbamate (6 mg, 0.025 mmol), Pd₂(dba)₃ (3.4 mg, 3.69 μmol), xantphos (4.3 mg, 7.39 μmol) and cesium carbonate (16 mg, 0.049 mmol) in dioxane (0.3 ml) was degassed and stirred at 100° C. for 4 h. The mixture was then cooled to r.t., diluted with CH₃CN and water and purified with prep-LCMS (XBridge C18 column, eluting with a gradient of acetonitrile/water containing 0.1% TFA, at flow rate of 60 mL/min). LCMS calculated for C₂₅H₃₀N₉O₅(M+H)⁺: m/z=536.2; found 536.3.

### Example 15. Methyl (3-((1-((1R,3R)-3-((methoxycarbonyl)amino)cyclopentyl)-3-methyl-2-oxo-2,3-dihydro-1H-imidazo[4,5-c]pyridin-6-yl)amino)-5-(1-methylpiperidin-4-yl)phenyl)carbamate

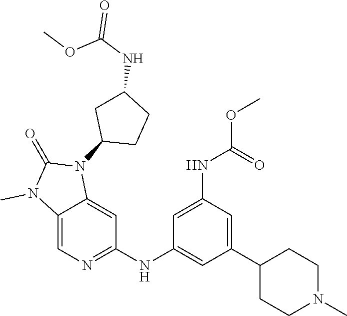

### Step 1. Methyl (3-amino-5-(1-methyl-1,2,3,6-tetrahydropyridin-4-yl)phenyl)carbamate

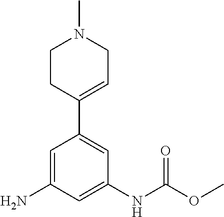 [SMILES: COC(=O)Nc1cc(N)cc(C2=CCN(C)CC2)c1]

The title compound was prepared according to the procedure described in Example 14 (Step 3), using 1-methyl-4-(4,4,5,5-tetramethyl-1,3,2-dioxaborolan-2-yl)-1,2,3,6-tetrahydropyridine instead of 1-methyl-4-(4,4,5,5-tetramethyl-1,3,2-dioxaborolan-2-yl)-1H-1,2,3-triazole. LCMS calculated for C₁₄H₂₀N₃O₂ (M+H)⁺: m/z=262.2; found 262.2.

### Step 2. Methyl (3-amino-5-(1-methylpiperidin-4-yl)phenyl)carbamate

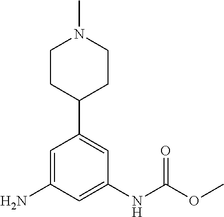 [SMILES: COC(=O)Nc1cc(N)cc(C2CCN(C)CC2)c1]

A mixture of methyl (3-amino-5-(1-methyl-1,2,3,6-tetrahydropyridin-4-yl)phenyl)carbamate (52 mg, 0.199 mmol) and Pd on carbon (10%, 21 mg, 0.020 mmol) in MeOH (5 ml) was stirred at r.t. under a balloon of H₂ for 1 h. The reaction mixture was then filtered and concentrated to give the desired compound. LCMS calculated for C₁₄H₂₂N₃O₂ (M+H)⁺: m/z=264.2; found 264.2.

### Step 3. Methyl (3-((1-((1R,3R)-3-((methoxycarbonyl)amino)cyclopentyl)-3-methyl-2-oxo-2,3-dihydro-1H-imidazo[4,5-c]pyridin-6-yl)amino)-5-(1-methylpiperidin-4-yl)phenyl)carbamate

The title compound was prepared according to the procedure described in Example 14 (Step 4), using methyl (3-amino-5-(1-methylpiperidin-4-yl)phenyl)carbamate instead of methyl (3-amino-5-(1-methyl-1H-1,2,3-triazol-4-yl)phenyl)carbamate. LCMS calculated for C₂₈H₃₈N₇O₅ (M+H)⁺: m/z=552.3; found 552.3.

### Example 16. Methyl ((1R,3R)-3-(6-((4-(3,3-difluoropiperidine-1-carbonyl)-8-(trifluoromethyl)quinolin-2-yl)amino)-3-(methyl-d3)-2-oxo-2,3-dihydro-1H-imidazo[4,5-c]pyridin-1-yl)cyclopentyl)carbamate

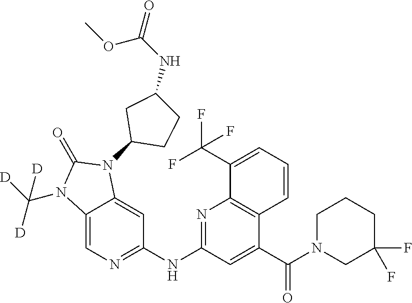 [SMILES: [2H]C([2H])([2H])n1c(=O)n([C@@H]2CC[C@@H](NC(=O)OC)C2)c2cc(Nc3cc(C(=O)N4CCCC(F)(F)C4)c4cccc(C(F)(F)F)c4n3)ncc21]

The title compound was prepared according to the procedures described in Example 3, with 3,3-difluoropiperidine replacing 1-methyl-1,6-diazaspiro[3.3]heptane in Step 7. LCMS calculated for C₃₀H₂₈D₃F₅N₇O₄ (M+H)⁺: m/z=651.3; found 651.3. ¹H NMR (400 MHz, DMSO) δ 11.29 (s, 1H), 8.58-8.37 (m, 1H), 8.28-8.17 (m, 1H), 7.93 (d, J=8.3 Hz, 1H), 7.89 (d, J=8.2 Hz, 1H), 7.84 (s, 1H), 7.70-7.60 (m, 1H), 7.36 (s, 1H), 7.31 (s, 1H), 4.93 (q, J=8.7 Hz, 1H), 4.26 (q, J=6.9 Hz, 1H), 4.14 (q, J=13.5 Hz, 1H), 3.95-3.78 (m, 1H), 3.74-3.61 (m, 1H), 3.55 (s, 3H), 2.43-2.31 (m, 1H), 2.28-2.05 (m, 5H), 2.01-1.84 (m, 2H), 1.68-1.49 (m, 2H).

### Example 17. Methyl ((1R,3R)-3-(6-((3-((R)-2,2-difluorocyclopropane-1-carboxamido)-5-(tetrahydro-2H-pyran-4-yl)phenyl)amino)-3-methyl-2-oxo-2,3-dihydro-1H-imidazo[4,5-c]pyridin-1-yl)cyclopentyl)carbamate

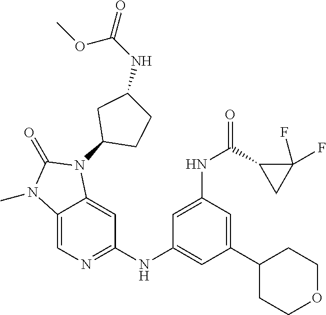 [SMILES: COC(=O)N[C@@H]1CC[C@@H](n2c(=O)n(C)c3cnc(Nc4cc(NC(=O)[C@H]5CC5(F)F)cc(C5CCOCC5)c4)cc32)C1]

### Step 1. 3-(3,6-Dihydro-2H-pyran-4-yl)-5-nitroaniline

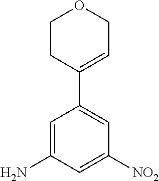 [SMILES: Nc1cc(C2=CCOCC2)cc([N+](=O)[O-])c1]

The title compound was prepared according to the procedure described in Example 14 (Step 3) using 3-bromo-5-nitroaniline and 2-(3,6-dihydro-2H-pyran-4-yl)-4,4,5,5-tetramethyl-1,3,2-dioxaborolane instead of methyl (3-amino-5-bromophenyl)carbamate and 1-methyl-4-(4,4,5,5-tetramethyl-1,3,2-dioxaborolan-2-yl)-1H-1,2,3-triazole. LCMS calculated for C₁₁H₁₃N₂O₃ (M+H)⁺: m/z=221.1; found 221.1.

### Step 2. Methyl ((1R,3R)-3-(6-((3-(3,6-dihydro-2H-pyran-4-yl)-5-nitrophenyl)amino)-3-methyl-2-oxo-2,3-dihydro-1H-imidazo[4,5-c]pyridin-1-yl)cyclopentyl)carbamate

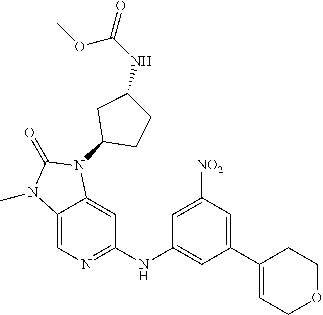 [SMILES: COC(=O)N[C@@H]1CC[C@@H](n2c(=O)n(C)c3cnc(Nc4cc(C5=CCOCC5)cc([N+](=O)[O-])c4)cc32)C1]

The title compound was prepared according to the procedure described in Example 14 (Step 4) using 3-(3,6-dihydro-2H-pyran-4-yl)-5-nitroaniline instead of methyl (3-amino-5-(1-methyl-1H-1,2,3-triazol-4-yl)phenyl)carbamate. LCMS calculated for C₂₅H₂₉N₆O₆ (M+H)⁺: m/z=509.2; found 509.2.

### Step 3. Methyl (1R,3R)-3-(6-((3-amino-5-(tetrahydro-2H-pyran-4-yl)phenyl)amino)-3-methyl-2-oxo-2,3-dihydro-1H-imidazo[4,5-c]pyridin-1-yl)cyclopentyl)carbamate

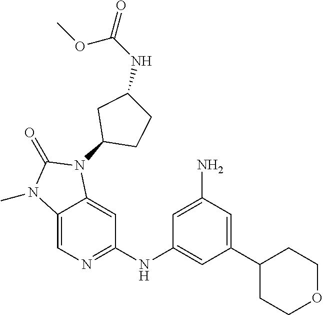 [SMILES: COC(=O)N[C@@H]1CC[C@@H](n2c(=O)n(C)c3cnc(Nc4cc(N)cc(C5CCOCC5)c4)cc32)C1]

The title compound was prepared according to the procedure described in Example 15 (Step 2) using methyl ((1R,3R)-3-(6-((3-(3,6-dihydro-2H-pyran-4-yl)-5-nitrophenyl)amino)-3-methyl-2-oxo-2,3-dihydro-1H-imidazo[4,5-c]pyridin-1-yl)cyclopentyl)carbamate instead of methyl (3-amino-5-(1-methyl-1,2,3,6-tetrahydropyridin-4-yl)phenyl)carbamate. LCMS calculated for C₂₅H₃₃N₆O₄ (M+H)⁺: m/z=481.3; found 481.2.

### Step 4. Methyl (IR. 3R)-3-(6-((3-((R)-2,2-difluorocyclopropane-1-carboxamido)-5-(tetrahydro-2H-pyran-4-yl)phenyl)amino)-3-methyl-2-oxo-2,3-dihydro-1H-imidazo[4,5-c]pyridin-1-yl)cyclopentyl)carbamate

A mixture of methyl ((1R,3R)-3-(6-((3-amino-5-(tetrahydro-2H-pyran-4-yl)phenyl)amino)-3-methyl-2-oxo-2,3-dihydro-1H-imidazo[4,5-c]pyridin-1-yl)cyclopentyl)carbamate (10 mg, 0.021 mmol), (R)-2,2-difluorocyclopropane-1-carboxylic acid (3 mg, 0.023 mmol), HATU (10 mg, 0.025 mmol) and DIPEA (11 μl, 0.062 mmol) in DMF (0.3 ml) was stirred at r.t. for 1 h. The mixture was diluted with CH₃CN and water and purified with prep-LCMS (XBridge C18 column, eluting with a gradient of acetonitrile/water containing 0.1% TFA, at flow rate of 60 mL/min). LCMS calculated for C₂₉H₃₅F₂N₆O₅ (M+H)⁺: m/z=585.3; found 585.3. ¹H NMR (500 MHz, DMSO-d₆) δ 10.44 (s, 1H), 9.50 (s, 1H), 8.00 (s, 1H), 7.67 (s, 1H), 7.33 (d, J=7.1 Hz, 1H), 7.11 (s, 1H), 7.05 (s, 1H), 6.83 (s, 1H), 4.90 (p, J=8.7 Hz, 1H), 4.19-4.12 (m, 1H), 3.99-3.92 (m, 2H), 3.54 (s, 3H), 3.48-3.39 (m, 2H), 3.33 (s, 3H), 2.88-2.77 (m, 1H), 2.77-2.69 (m, 1H), 2.29-2.21 (m, 1H), 2.18-1.83 (m, 6H), 1.76-1.52 (m, 5H).

### Example 18. Methyl ((1R,3R)-3-(6-((6-((3,3-difluorocyclopentyl)amino)-8-(trifluoromethyl)quinolin-2-yl)amino)-3-(methyl-d3)-2-oxo-2,3-dihydro-1H-imidazo[4,5-c]pyridin-1-yl)cyclopentyl)carbamate

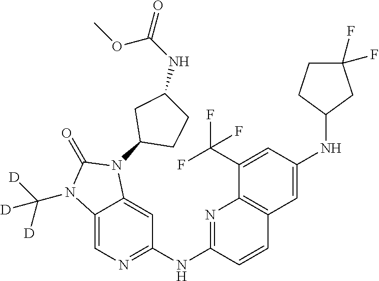 [SMILES: [2H]C([2H])([2H])n1c(=O)n([C@@H]2CC[C@@H](NC(=O)OC)C2)c2cc(Nc3ccc4cc(NC5CCC(F)(F)C5)cc(C(F)(F)F)c4n3)ncc21]

### Step 1. Ethyl 3-(2-amino-5-chloro-3-(trifluoromethyl)phenyl)acrylate

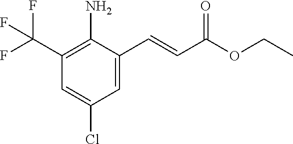 [SMILES: CCOC(=O)/C=C/c1cc(Cl)cc(C(F)(F)F)c1N]

A vial containing 2-bromo-4-chloro-6-(trifluoromethyl)aniline (933 mg, 3.4 mmol), ethyl acrylate (0.724 mL, 6.8 mmol), DIPEA (1.8 mL, 10.2 mmol), tri-o-tolylphosphane (207 mg, 0.68 mmol) and Pd(OAc)₂ was evacuated and backfilled with nitrogen. DMF (7 mL) was added to the reaction mixture, which was then stirred at 100° C. overnight. The reaction was quenched by the addition of water and extracted with EtOAc. The organic layer was dried over sodium sulfate, filtered, and the solvents were evaporated in vacuo. The crude material was purified by Biotage Isolera. LCMS calculated for C₁₂H₁₂ClF₃NO₂ (M+H)⁺: m/z=294.0; found 294.0.

### Step 2. 6-Chloro-8-(trifluoromethyl)quinolin-2(1H)-one

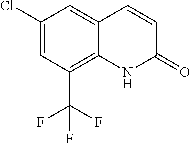 [SMILES: O=c1ccc2cc(Cl)cc(C(F)(F)F)c2[nH]1]

To a suspension of ethyl 3-(2-amino-5-chloro-3-(trifluoromethyl)phenyl)acrylate (587 mg, 2 mmol) in 1,4-dioxane (4 mL) was added HCl (12 M, 3 mL). The mixture was stirred at 100° C. for 5 h. The reaction mixture was cooled to r.t and quenched by the addition of water. The solid was collected by filtration and used in the next step without further purification. LCMS calculated for C₁₀H₆ClF₃NO (M+H)⁺: m/z=248.0; found 248.0.

### Step 3. 2,6-Dichloro-8-(trifluoromethyl)quinoline

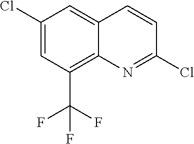 [SMILES: FC(F)(F)c1cc(Cl)cc2ccc(Cl)nc12]

A mixture of 6-chloro-8-(trifluoromethyl)quinolin-2(1H)-one (495 mg, 2 mmol) and phosphoryl trichloride (5 mL) was stirred at 110° C. for 1 h. The reaction mixture was allowed to cool to r.t and concentrated in vacuo. The residue was quenched with ice water. The solid was collected by filtration and used in the next step without further purification. LCMS calculated for C₁₀H₅Cl₂F₃N (M+H)⁺: m/z=266.0; found 266.0.

### Step 4. 6-Chloro-8-(trifluoromethyl)quinolin-2-amine

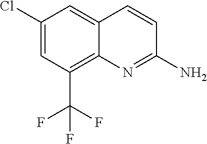 [SMILES: Nc1ccc2cc(Cl)cc(C(F)(F)F)c2n1]

To a microwave vial containing 2,6-dichloro-8-(trifluoromethyl)quinoline (400 mg, 1.5 mmol) and 1,4-dioxane (4 mL) was added ammonia hydroxide (4 mL), which was then microwaved at 130° C. for 2 h. The reaction mixture was cooled to r.t and extracted with EtOAc. The organic layer was dried over sodium sulfate, filtered, and the solvents were evaporated in vacuo. The crude material was used in the next step without further purification. LCMS calculated for C₁₀H₇ClF₃N₂ (M+H)⁺: m/z=247.0; found 247.0.

### Step 5. Methyl ((1R,3R)-3-(6-((6-chloro-8-(trifluoromethyl) quinolin-2-yl)amino)-3-(methyl-d3)-2-oxo-2,3-dihydro-1H-imidazo[4,5-c]pyridin-1-yl)cyclopentyl)carbamate

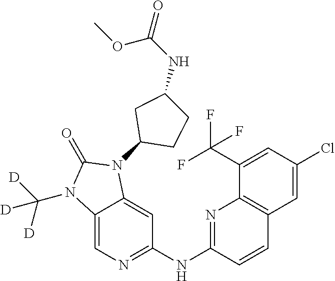 [SMILES: [2H]C([2H])([2H])n1c(=O)n([C@@H]2CC[C@@H](NC(=O)OC)C2)c2cc(Nc3ccc4cc(Cl)cc(C(F)(F)F)c4n3)ncc21]

A reaction vial containing methyl ((1R,3R)-3-(6-chloro-3-(methyl-d3)-2-oxo-2,3-dihydro-1H-imidazo[4,5-c]pyridin-1-yl)cyclopentyl)carbamate (492 mg, 1.5 mmol), 6-chloro-8-(trifluoromethyl)quinolin-2-amine (407 mg, 1.7 mmol), tris(dibenzylideneacetone)dipalladium(0) (138 mg, 0.15 mmol), Xantphos (167 mg, 0.3 mmol) and cesium carbonate (1.5 g, 4.5 mmol) was evacuated and backfilled with nitrogen. 1,4-Dioxane (10 mL) was added to the reaction mixture, which was then stirred at 110° C. overnight. The reaction mixture was cooled to r.t and diluted with EtOAc. The organic layer was subsequently washed with water and brine, dried over sodium sulfate, filtered, and the solvents were evaporated in vacuo. The crude material was purified by Biotage Isolera. LCMS calculated for C₂₄H₂₀D₃ClF₃N₆O₃ (M+H)⁺: m/z=538.2; found 538.2.

### Step 6. Methyl (1R,3R)-3-(6-((6-((3,3-difluorocyclopentyl)amino)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

A reaction vial containing methyl ((1R,3R)-3-(6-((6-chloro-8-(trifluoromethyl)quinolin-2-yl)amino)-3-(methyl-d3)-2-oxo-2,3-dihydro-1H-imidazo[4,5-c]pyridin-1-yl)cyclopentyl)carbamate (10 mg, 0.019 mmol), 3,3-difluorocyclopentan-1-amine hydrochloride (9 mg, 0.056 mmol), xantphos-Pd-G3 (2 mg, 0.002 mmol) and cesium carbonate (30 mg, 0.093 mmol) was evacuated and backfilled with nitrogen. 1,4-Dioxane (0.5 mL) was added to the reaction mixture, which was then stirred at 110° C. overnight. The reaction mixture was allowed to cool to r.t., diluted with MeCN and purified by prep-LCMS (XBridge C18 column, eluting with a gradient of acetonitrile/water containing 0.1% TFA, at flow rate of 60 mL/min. LCMS calculated for C₂₉H₂₈D₃F₅N₇O₃ (M+H)⁺: m/z=623.3; found 623.3. ¹H NMR (400 MHZ, DMSO) δ 11.50 (s, 1H), 8.37 (s, 1H), 8.27 (d, J=8.8 Hz, 1H), 7.64 (d, J=2.3 Hz, 1H), 7.37 (s, 1H), 7.31 (d, J=9.0 Hz, 1H), 7.13 (s, 1H), 6.68 (s, 1H), 4.93 (p, J=8.5 Hz, 1H), 4.25 (q, J=6.8 Hz, 1H), 4.07 (t, J=7.0 Hz, 1H), 3.56 (s, 3H), 2.91-2.65 (m, 1H), 2.42-1.55 (m, 12H). 19F NMR (376 MHz, DMSO) δ−60.6, −87.9.

### Example 19. Methyl ((1R,3R)-3-(6-((3-(cyclobutanecarboxamido)-5-(tetrahydro-2H-pyran-4-yl)phenyl)amino)-3-methyl-2-oxo-2,3-dihydro-1H-imidazo[4,5-c]pyridin-1-yl)cyclopentyl)carbamate

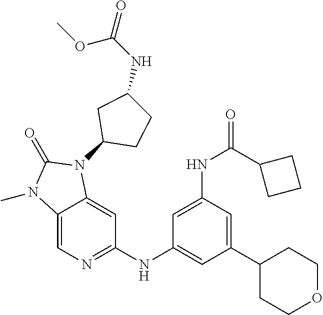 [SMILES: COC(=O)N[C@@H]1CC[C@@H](n2c(=O)n(C)c3cnc(Nc4cc(NC(=O)C5CCC5)cc(C5CCOCC5)c4)cc32)C1]

The title compound was prepared according to the procedure described in Example 17, using cyclobutanecarboxylic acid instead of (R)-2,2-difluorocyclopropane-1-carboxylic acid. LCMS calculated for C₃₀H₃₉N₆O₅ (M+H)⁺: m/z=563.3; found 563.4. ¹H NMR (500 MHz, DMSO-d₆) δ 9.75 (s, 1H), 9.59 (s, 1H), 8.00 (s, 1H), 7.71 (s, 1H), 7.32 (s, 1H), 7.14 (s, 1H), 6.97 (s, 1H), 6.85 (s, 1H), 4.91 (p, J=8.6 Hz, 1H), 4.19-4.11 (m, 1H), 3.98-3.92 (m, 2H), 3.53 (s, 3H), 3.48-3.40 (m, 2H), 3.33 (s, 3H), 3.28-3.17 (m, 1H), 2.75-2.67 (m, 1H), 2.30-2.17 (m, 3H), 2.16-2.05 (m, 4H), 2.02-1.77 (m, 4H), 1.75-1.55 (m, 5H).

### Example 20. Methyl ((1R,3R)-3-(3-(methyl-d3)-6-((6-(1-methyl-1H-pyrazol-4-yl)-8-(trifluoromethyl)quinolin-2-yl)amino)-2-oxo-2,3-dihydro-1H-imidazo[4,5-c]pyridin-1-yl)cyclopentyl)carbamate

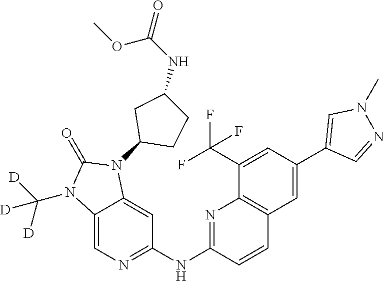 [SMILES: [2H]C([2H])([2H])n1c(=O)n([C@@H]2CC[C@@H](NC(=O)OC)C2)c2cc(Nc3ccc4cc(-c5cnn(C)c5)cc(C(F)(F)F)c4n3)ncc21]

A reaction vial containing methyl ((1R,3R)-3-(6-((6-chloro-8-(trifluoromethyl)quinolin-2-yl)amino)-3-(methyl-d3)-2-oxo-2,3-dihydro-1H-imidazo[4,5-c]pyridin-1-yl)cyclopentyl)carbamate (10 mg, 0.019 mmol), (1-methyl-1H-pyrazol-4-yl)boronic acid (5 mg, 0.037 mmol), XPhos-Pd-G2 (2 mg, 0.002 mmol) and potassium phosphate tribasic (13 mg, 0.06 mmol) was evacuated and backfilled with nitrogen. 1,4-Dioxane (0.4 mL) and water (0.1 mL) were added to the reaction mixture, which was then stirred at 90° C. for 30 min. The reaction mixture was allowed to cool to r.t, diluted with MeCN and purified by prep-LCMS (XBridge C18 column, eluting with a gradient of acetonitrile/water containing 0.1% TFA, at flow rate of 60 mL/min. LCMS calculated for C₂₈H₂₅D₃F₃N₈O₃ (M+H)⁺: m/z=584.2; found 584.2. ¹H NMR (400 MHZ, DMSO) δ 11.30 (s, 1H), 8.49-8.26 (m, 5H), 8.08 (s, 1H), 7.84 (s, 1H), 7.44 (d, J=9.0 Hz, 1H), 7.36 (d, J=7.0 Hz, 1H), 4.91 (p, J=8.3 Hz, 1H), 4.26 (q, J=6.9 Hz, 1H), 3.92 (s, 3H), 3.56 (s, 3H), 2.43-2.31 (m, 1H), 2.29-1.84 (m, 4H), 1.74-1.55 (m, 1H). 19F NMR (376 MHz, DMSO) δ-60.1.

### Example 21. Methyl (3-((1-((1R,3R)-3-((methoxycarbonyl)amino)cyclopentyl)-3-methyl-2-oxo-2,3-dihydro-1H-imidazo[4,5-c]pyridin-6-yl)amino)-5-(tetrahydro-2H-pyran-3-yl)phenyl)carbamate

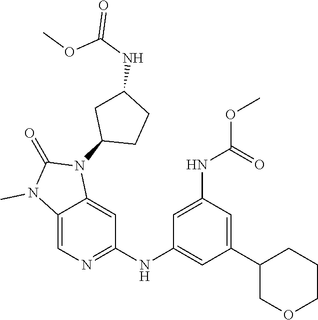 [SMILES: COC(=O)Nc1cc(Nc2cc3c(cn2)n(C)c(=O)n3[C@@H]2CC[C@@H](NC(=O)OC)C2)cc(C2CCCOC2)c1]

### Step 1. Methyl (3-amino-5-(5,6-dihydro-2H-pyran-3-yl)phenyl)carbamate

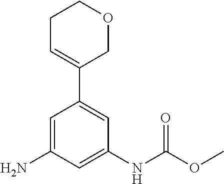 [SMILES: COC(=O)Nc1cc(N)cc(C2=CCCOC2)c1]

The title compound was prepared according to the procedure described in Example 14 (Step 3), using 2-(5,6-dihydro-2H-pyran-3-yl)-4,4,5,5-tetramethyl-1,3,2-dioxaborolane instead of 1-methyl-4-(4,4,5,5-tetramethyl-1,3,2-dioxaborolan-2-yl)-1H-1,2,3-triazole. LCMS calculated for C₁₃H₁₇N₂O₃ (M+H)⁺: m/z=249.1; found 249.2.

### Step 2. Methyl (3-amino-5-(tetrahydro-2H-pyran-3-yl)phenyl)carbamate

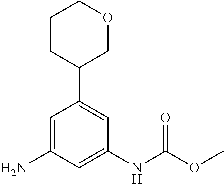 [SMILES: COC(=O)Nc1cc(N)cc(C2CCCOC2)c1]

The title compound was prepared according to the procedure described in Example 15 (Step 2), using methyl (3-amino-5-(5,6-dihydro-2H-pyran-3-yl)phenyl)carbamate instead of methyl (3-amino-5-(1-methyl-1,2,3,6-tetrahydropyridin-4-yl)phenyl)carbamate. LCMS calculated for C₁₃H₁₉N₂O₃ (M+H)⁺: m/z=251.1; found 251.2.

### Step 3. Methyl (3-((1-((1R,3R)-3-((methoxycarbonyl)amino)cyclopentyl)-3-methyl-2-oxo-2,3-dihydro-1H-imidazo[4,5-c]pyridin-6-yl)amino)-5-(tetrahydro-2H-pyran-3-yl)phenyl)carbamate

The title compound was prepared according to the procedure described in Example 14 (Step 4), using methyl (3-amino-5-(5,6-dihydro-2H-pyran-3-yl)phenyl)carbamate instead of methyl (3-amino-5-(1-methyl-1H-1,2,3-triazol-4-yl)phenyl)carbamate. LCMS calculated for C₂₇H₃₅N₆O₆ (M+H)⁺: m/z=539.3; found 539.2.

### Example 22. Methyl ((1R,3R)-3-(6-((6-((4-cyanocyclohexyl)oxy)pyridin-2-yl)amino)-3-methyl-2-oxo-2,3-dihydro-1H-imidazo[4,5-c]pyridin-1-yl)cyclopentyl)carbamate

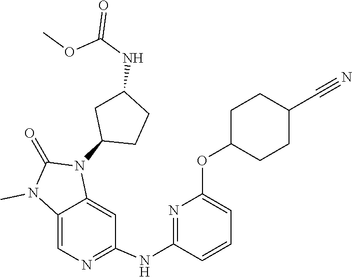 [SMILES: COC(=O)N[C@@H]1CC[C@@H](n2c(=O)n(C)c3cnc(Nc4cccc(OC5CCC(C#N)CC5)n4)cc32)C1]

### Step 1. 4-((6-Aminopyridin-2-yl)oxy)cyclohexane-1-carbonitrile

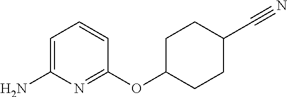 [SMILES: N#CC1CCC(Oc2cccc(N)n2)CC1]

The title compound was prepared according to the procedures described in Example 23, with 4-hydroxycyclohexane-1-carbonitrile replacing quinolin-6-ol in Step 1. LCMS calculated for C₁₂H₁₆N₃O (M+H)⁺: m/z=218.1; found 218.1.

### Step 2. Methyl ((1R,3R)-3-(6-((6-((4-cyanocyclohexyl)oxy)pyridin-2-yl)amino)-3-methyl-2-oxo-2,3-dihydro-1H-imidazo[4,5-c]pyridin-1-yl)cyclopentyl)carbamate

The title compound was prepared according to the procedures described in Example 23, with 4-((6-aminopyridin-2-yl)oxy)cyclohexane-1-carbonitrile replacing 6-(quinolin-6-yloxy)pyridin-2-amine in Step 2. LCMS calculated for C₂₋₆H₃₂N₇O₄ (M+H)⁺: m/z=506.2; found 506.2.

### Example 23. Methyl ((1R,3R)-3-(3-methyl-2-oxo-6-((6-(quinolin-6-yloxy)pyridin-2-yl)amino)-2,3-dihydro-1H-imidazo[4,5-c]pyridin-1-yl)cyclopentyl)carbamate

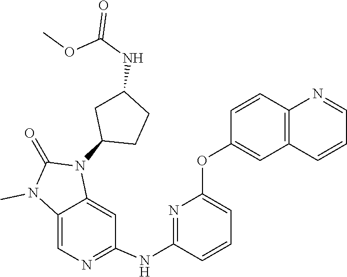 [SMILES: COC(=O)N[C@@H]1CC[C@@H](n2c(=O)n(C)c3cnc(Nc4cccc(Oc5ccc6ncccc6c5)n4)cc32)C1]

### Step 1. 6-(Quinolin-6-yloxy)pyridin-2-amine

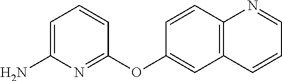 [SMILES: Nc1cccc(Oc2ccc3ncccc3c2)n1]

To a microwave vial containing 6-nitropyridin-2-amine (30 mg, 0.216 mmol), quinolin-6-ol (80 mg, 0.647 mmol) and potassium carbonate (89 mg, 0.647 mmol) was added DMF (0.7 mL), which was then microwaved at 180° C. for 1 h. The reaction mixture was cooled to r.t and extracted with EtOAc. The organic layer was dried over sodium sulfate, filtered, and the solvents were evaporated in vacuo. The crude material was purified by Biotage Isolera. LCMS calculated for C₁₄H₁₂N₃O (M+H)⁺: m/z=238.1; found 238.1.

### Step 2. Methyl (1R,3R)-3-(3-methyl-2-oxo-6-(6-(quinolin-6-yloxy)pyridin-2-yl)amino)-2,3-dihydro-1H-imidazo[4,5-c]pyridin-1-yl)cyclopentyl)carbamate

A reaction vial containing methyl ((1R,3R)-3-(6-chloro-3-methyl-2-oxo-2,3-dihydro-1H-imidazo[4,5-c]pyridin-1-yl)cyclopentyl)carbamate (10 mg, 0.031 mmol), 6-(quinolin-6-yloxy)pyridin-2-amine (13 mg, 0.062 mmol), tris(dibenzylideneacetone)dipalladium(0) (3 mg, 0.003 mmol), Xantphos (4 mg, 0.006 mmol) and cesium carbonate (20 mg, 0.062 mmol) was evacuated and backfilled with nitrogen. 1,4-Dioxane (0.5 mL) was added to the reaction mixture, which was then stirred at 110° C. overnight. The reaction mixture was allowed to cool to r.t, diluted with MeCN and purified by prep-LCMS (XBridge C18 column, eluting with a gradient of acetonitrile/water containing 0.1% TFA, at flow rate of 60 mL/min. LCMS calculated for C₂₈H₂₈N₇O₄ (M+H)⁺: m/z=526.2; found 526.2. ¹H NMR (400 MHZ, DMSO) δ 10.64 (s, 1H), 8.97 (d, J=4.4 Hz, 1H), 8.46 (d, J=8.4 Hz, 1H), 8.18 (d, J=9.0 Hz, 1H), 7.89 (t, J=7.9 Hz, 1H), 7.83 (d, J=2.6 Hz, 1H), 7.75 (d, J=9.1 Hz, 1H), 7.69-7.57 (m, 2H), 7.31 (d, J=6.8 Hz, 1H), 7.21 (s, 1H), 7.18 (d, J=8.2 Hz, 1H), 6.68 (d, J=7.7 Hz, 1H), 4.77 (p, J=8.7 Hz, 1H), 4.14 (q, J=6.8 Hz, 1H), 3.53 (s, 3H), 3.26 (s, 3H), 2.27-2.06 (m, 2H), 1.98-1.84 (m, 2H), 1.86-1.72 (m, 1H), 1.62-1.43 (m, 1H).

### Example 24. Methyl (3-((1-((1r,3r)-3-(cyclopropanecarboxamido)cyclobutyl)-3-methyl-2-oxo-2,3-dihydro-1H-imidazo[4,5-c]pyridin-6-yl)amino)-5-(1-methyl-1H-pyrazol-4-yl)phenyl)carbamate

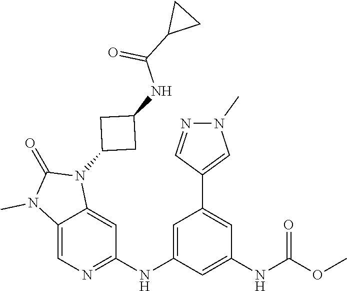

### Step 1. Methyl (3-amino-5-bromophenyl)carbamate

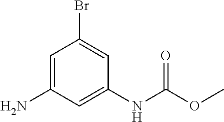

To a reaction flask containing 3-bromo-5-nitroaniline (1.44 g, 7.16 mmol) in DCM (60 mL) was added N-ethyl-N-isopropylpropan-2-amine (1.38 mL, 7.88 mmol) and methyl carbonochloridate (0.61 mL, 7.88 mmol). The reaction mixture was allowed to stir at r.t. for 1 h, after which the solvent was evaporated in vacuo. The crude material was dissolved in THF (30 mL) and water (7.5 mL). Ammonium chloride (3.83 g, 71.6 mmol) and zinc powder (4.68 g, 71.6 mmol) were added to the reaction mixture which was then heated to 40° C. for 2 h and then cooled to r.t. The reaction mixture was diluted with EtOAc and filtered through celite pad. The organic layer was subsequently washed with water and brine, dried over sodium sulfate, filtered, and the solvents were evaporated in vacuo. The crude material was purified by Biotage Isolera to give the product. LCMS calculated for C₈H₁₀BrN₂O₂ (M+H)⁺: m/z=245.0; found 245.0.

### Step 2. Methyl (3-amino-5-(1-methyl-1H-pyrazol-4-yl)phenyl) carbamate

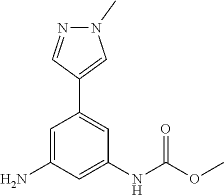 [SMILES: COC(=O)Nc1cc(N)cc(-c2cnn(C)c2)c1]

A reaction flask containing methyl (3-amino-5-bromophenyl)carbamate (1.62 g, 6.59 mmol), (1-methyl-1H-pyrazol-4-yl)boronic acid (1.0 g, 7.91 mmol), potassium phosphate (2.8 g, 13.18 mmol) and [1, l′-bis(diphenylphosphino)ferrocene]-dichloropalladium(II) (0.54 g, 659.0 μmol) was evacuated and backfilled with nitrogen. Acetonitrile (55 mL) and water (11 mL) were added to the reaction mixture, which was then stirred at 80° C. for 2 h. The reaction mixture was cooled to r.t and was diluted with EtOAc. The organic layer was subsequently washed with water and brine, dried over sodium sulfate, filtered, and the solvents were evaporated in vacuo. The crude material was purified by Biotage Isolera to give the product. LCMS calculated for C₁₂H₁₅N₄O₂ (M+H)⁺: m/z=247.1; found 247.1.

### Step 3. tert-Butyl ((1r,3r)-3-(6-chloro-2-oxo-2,3-dihydro-1H-imidazo[4,5-c]pyridin-1-yl)cyclobutyl)carbamate

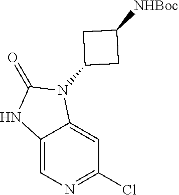 [SMILES: CC(C)(C)OC(=O)N[C@H]1C[C@H](n2c(=O)[nH]c3cnc(Cl)cc32)C1]

To a mixture of 2,4-dichloro-5-nitropyridine (272 mg, 1.41 mmol) and tert-butyl ((1r,3r)-3-aminocyclobutyl)carbamate (250 mg, 1.34 mmol) in THF (9 mL) was added N-ethyl-N-isopropylpropan-2-amine (0.28 mL, 1.61 mmol). The reaction mixture was heated to 55° C. for 1 h and then cooled to r.t. Then water (2.0 mL), ammonium chloride (718 mg, 13.42 mmol), and zinc powder (878 mg, 13.42 mmol) were added to the reaction mixture which was heated to 40° C. for 2 h and then cooled to r.t. The reaction mixture was diluted with EtOAc and filtered through celite pad. The organic layer was subsequently washed with water and brine, dried over sodium sulfate, filtered, and the solvents were evaporated in vacuo. In a separate reaction vial, the crude material was dissolved in acetonitrile (13 mL) then N-ethyl-N-isopropylpropan-2-amine (0.24 mL, 1.34 mmol) and bis(2,5-dioxopyrrolidin-1-yl) carbonate (378 mg, 1.48 mmol) were added to the reaction mixture. The reaction mixture was allowed to stir at r.t. for 16 h, after which it was diluted with EtOAc. The organic layer was subsequently washed with water and brine, dried over sodium sulfate, filtered, and the solvents were evaporated in vacuo. The crude material was purified by Biotage Isolera to give the product. LCMS calculated for C₁₅H₂₀ClN₄O₃ (M+H)⁺: m/z=339.1; found 339.1.

### Step 4. tert-Butyl ((1r,3r)-3-(6-chloro-3-methyl-2-oxo-2,3-dihydro-1H-imidazo[4,5-c]pyridin-1-yl)cyclobutyl)carbamate

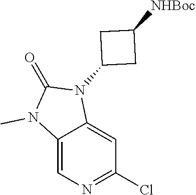 [SMILES: Cn1c(=O)n([C@H]2C[C@H](NC(=O)OC(C)(C)C)C2)c2cc(Cl)ncc21]

To a reaction vial containing tert-butyl ((1r,3r)-3-(6-chloro-2-oxo-2,3-dihydro-1H-imidazo[4,5-c]pyridin-1-yl)cyclobutyl)carbamate (440 mg, 1.3 mmol) and THF (13 mL) was added cesium carbonate (1.27 g, 3.9 mmol) and iodomethane (0.2 mL, 3.25 mmol) and allowed to stir at r.t. for 16 h. The reaction mixture was diluted with DCM and filtered through a celite pad. The solvents were evaporated in vacuo and then the crude material was purified by Biotage Isolera to give the product. LCMS calculated for C₁₆H₂₂ClN₄O₃ (M+H)⁺: m/z=353.1; found 353.1.

### Step 5. Methyl (3-((1-((1r,3r)-3-((tert-butoxycarbonyl)amino)cyclobutyl)-3-methyl-2-oxo-2,3-dihydro-1H-imidazo[4,5-c]pyridin-6-yl)amino)-5-(1-methyl-1H-pyrazol-4-yl)phenyl)carbamate

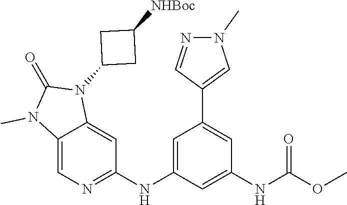

To a reaction vial were added tert-butyl ((1r,3r)-3-(6-chloro-3-methyl-2-oxo-2,3-dihydro-1H-imidazo[4,5-c]pyridin-1-yl)cyclobutyl)carbamate (400 mg, 1.13 mmol), methyl (3-amino-5-(1-methyl-1H-pyrazol-4-yl)phenyl)carbamate (279 mg, 1.13 mmol, Example 24, Step 2), XantPhos Pd G3 (54 mg, 0.06 mmol), and cesium carbonate (739 mg, 2.27 mmol). This was evacuated and backfilled with nitrogen 3 times. Subsequently 1,4-dioxane (14 mL) was added. The vial was sealed and heated to 100° C. for 2 h. Upon completion, the reaction was diluted with DCM, filtered through Celite and concentrated in vacuo. The crude material was purified by Biotage Isolera to give the desired product. LCMS calculated for C₂₈H₃₅N₈O₅ (M+H)⁺: m/z=563.3; found 563.3.

### Step 6. Methyl (3-(1-(1r,3r)-3-(cyclopropanecarboxamido)cyclobutyl)-3-methyl-2-oxo-2,3-dihydro-1H-imidazo[4,5-c]pyridin-6-yl)amino)-5-(1-methyl-1H-pyrazol-4-yl)phenyl)carbamate

A reaction vial containing methyl (3-((1-((1r,3r)-3-((tert-butoxycarbonyl)amino)cyclobutyl)-3-methyl-2-oxo-2,3-dihydro-1H-imidazo[4,5-c]pyridin-6-yl)amino)-5-(1-methyl-1H-pyrazol-4-yl)phenyl)carbamate (15 mg, 0.03 mmol) was treated with a solution of 4:1 DCM/TFA (2 mL) and stirred at r.t. for 4 h. The solvents were evaporated in vacuo and then acetonitrile (0.65 mL), N-ethyl-N-isopropylpropan-2-amine (28 μL, 0.16 mmol), cyclopropanecarboxylic acid (7.8 μL, 0.1 mmol), 2,4,6-tripropyl-1,3,5,2,4,6-trioxatriphosphinane 2,4,6-trioxide (77 μL, 0.13 mmol, 50 wt. % in ethyl acetate) were added to the reaction vial. The reaction mixture was allowed to stir at r.t. for 1 h, after which the solvent was evaporated in vacuo and the crude material was diluted with MeCN and purified by prep-LCMS (XBridge C18 column, eluting with a gradient of acetonitrile/water containing 0.1% TFA, at flow rate of 60 mL/min. LCMS calculated for C₂₇H₃₁N₈O₄(M+H)⁺: m/z=531.3; found: 531.3

### Example 25. Methyl (3-((1-(6-((ethoxycarbonyl)amino)spiro[3.3]heptan-2-yl)-3-methyl-2-oxo-2,3-dihydro-1H-imidazo[4,5-c]pyridin-6-yl)amino)-5-(1-methyl-1H-pyrazol-4-yl)phenyl)carbamate

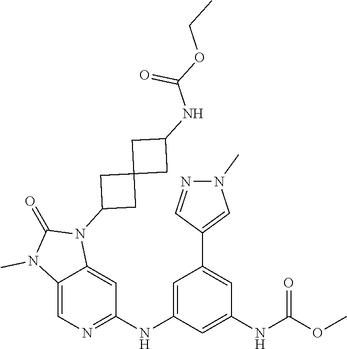

### Step 1. Methyl (3-((1-(6-((tert-butoxycarbonyl)amino)spiro[3.3]heptan-2-yl)-3-methyl-2-oxo-2,3-dihydro-1H-imidazo[4,5-c]pyridin-6-yl)amino)-5-(1-methyl-1H-pyrazol-4-yl)phenyl)carbamate

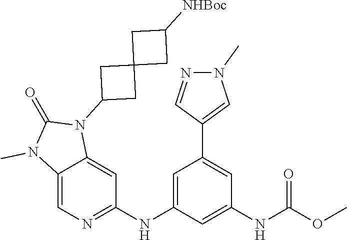

The title compound was prepared according to the procedures described in Example 24, Steps 3-5 with tert-butyl (6-aminospiro[3.3]heptan-2-yl)carbamate replacing tert-butyl ((1r,3r)-3-aminocyclobutyl)carbamate in Step 3. LCMS calculated for C₃₁H₃₉N₈O₅ (M+H)⁺: m/z=603.3; found: 603.3.

### Step 2. Methyl (3-((1-(6-((ethoxycarbonyl)amino)spiro[3.3]heptan-2-yl)-3-methyl-2-oxo-2,3-dihydro-1H-imidazo[4,5-c]pyridin-6-yl)amino)-5-(1-methyl-1H-pyrazol-4-yl)phenyl)carbamate

A reaction vial containing methyl (3-((1-(6-((tert-butoxycarbonyl)amino)spiro[3.3]heptan-2-yl)-3-methyl-2-oxo-2,3-dihydro-1H-imidazo[4,5-c]pyridin-6-yl)amino)-5-(1-methyl-1H-pyrazol-4-yl)phenyl)carbamate (15 mg, 0.03 mmol) was treated with a solution of 4:1 DCM/TFA (2 mL) and stirred at r.t. for 4 h. The solvents were evaporated in vacuo and then DCM (0.6 mL) and N-ethyl-N-isopropylpropan-2-amine (26 μL, 0.15 mmol) were added to the reaction vial and cooled to 0° C. and ethyl chloroformate (3 μL, 0.033 mmol) was added. The reaction was allowed to stir at 0° C. for 10 min, then quenched with MeOH (2 ml) and solvents were evaporated in vacuo. The crude material was diluted with MeCN and purified by prep-LCMS (XBridge C18 column, eluting with a gradient of acetonitrile/water containing 0.1% TFA, at flow rate of 60 mL/min. LCMS calculated for C₂₉H₃₅N₈O₅ (M+H)⁺: m/z=575.3; found: 575.3.

### Example 26. Methyl (3-((1-(6-(cyclopropanesulfonamido)spiro[3.3]heptan-2-yl)-3-methyl-2-oxo-2,3-dihydro-1H-imidazo[4,5-c]pyridin-6-yl)amino)-5-(1-methyl-1H-pyrazol-4-yl)phenyl)carbamate

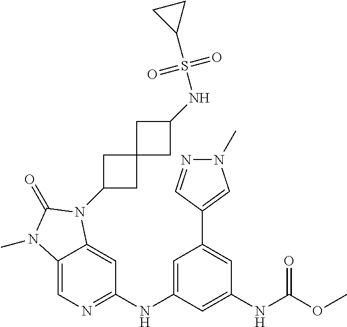

The title compound was prepared according to the procedures described in Example 25, Steps 1-2 with cyclopropanesulfonyl chloride replacing ethyl chloroformate in Step 2. LCMS calculated for C₂₉H₃₆N₈O₅S (M+H)⁺: m/z=607.2; found: 607.2.

### Example 27. Methyl 6-(6-((3-((methoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)-2-azaspiro[3.3]heptane-2-carboxylate

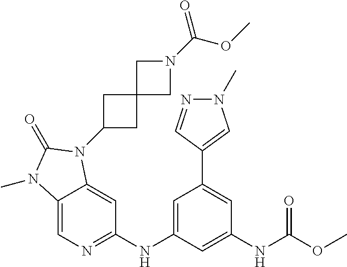

### Step 1. tert-Butyl 6-(6-((3-((methoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)-2-azaspiro[3.3]heptane-2-carboxylate

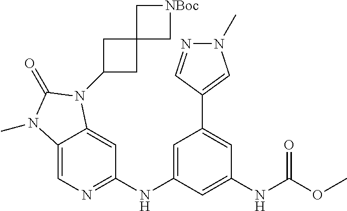

The title compound was prepared according to the procedures described in Example 24, Steps 3-5 with tert-butyl 6-amino-2-azaspiro[3.3]heptane-2-carboxylate replacing tert-butyl ((1r,3r)-3-aminocyclobutyl)carbamate in Step 3. LCMS calculated for C₃₀H₃₇N₈O₅ (M+H)⁺: m/z=588.3; found: 588.3.

### Step 2. Methyl 6-(6-((3-((methoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)-2-azaspiro[3.3]heptane-2-carboxylate

A reaction vial containing tert-butyl 6-(6-((3-((methoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)-2-azaspiro[3.3]heptane-2-carboxylate (15 mg, 0.03 mmol) was treated with a solution of 4:1 DCM/TFA (2 mL) and stirred at r.t. for 4 h. The solvents were evaporated in vacuo and then DCM (0.6 mL) and N-ethyl-N-isopropylpropan-2-amine (27 μL, 0.15 mmol) were added to the reaction vial and cooled to 0° C. and methyl chloroformate (2.6 μL, 0.034 mmol) was added. The reaction was allowed to stir at 0° C. for 10 min, then quenched with MeOH (2 ml) and solvents were evaporated in vacuo. The crude material was diluted with MeCN and purified by prep-LCMS (XBridge C18 column, eluting with a gradient of acetonitrile/water containing 0.1% TFA, at flow rate of 60 mL/min. LCMS calculated for C₂₇H₃₁N₈O₅ (M+H)⁺: m/z=547.2; found: 547.2.

### Example 28. Methyl (3-((1-((1r,3r)-3-cyanocyclobutyl)-3-methyl-2-oxo-2,3-dihydro-1H-imidazo[4,5-c]pyridin-6-yl)amino)-5-(1-methyl-1H-pyrazol-4-yl)phenyl)carbamate

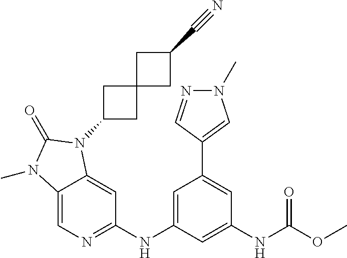

The title compound was prepared according to the procedures described in Example 24, Steps 3-5 with (1r,3r)-3-aminocyclobutane-1-carbonitrile hydrochloride replacing tert-butyl ((1r,3r)-3-aminocyclobutyl)carbamate in Step 3, and with purification by prep-LCMS (XBridge C18 column, eluting with a gradient of acetonitrile/water containing 0.1% TFA, at flow rate of 60 mL/min replacing purification by Biotage Isolera to give the desired product. LCMS calculated for C₂₄H₂₅N₈O₃ (M+H)⁺: m/z=473.2; found: 473.2. ¹H NMR (400 MHZ, DMSO) δ 9.69 (s, 1H), 9.39 (s, 1H), 8.00 (d, J=3.0 Hz, 2H), 7.70 (s, 1H), 7.44 (s, 1H), 7.29 (d, J=10.4 Hz, 2H), 6.98 (s, 1H), 5.12 (p, J=8.7 Hz, 1H), 3.88 (s, 3H), 3.69 (s, 3H), 3.56-3.45 (m, 1H), 3.33 (s, 3H), 3.26-3.14 (m, 2H), 2.78-2.67 (m, 2H).

### Example 29. Methyl (3-((1-((1r,3r)-3-((methoxycarbonyl)amino)-3-methylcyclobutyl)-3-methyl-2-oxo-2,3-dihydro-1H-imidazo[4,5-c]pyridin-6-yl)amino)-5-(1-methyl-1H-pyrazol-4-yl)phenyl)carbamate

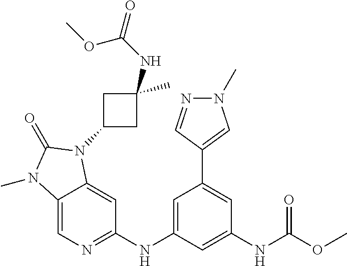

### Step 1. Methyl (3-((1-((1r,3r)-3-((tert-butoxycarbonyl)amino)-3-methylcyclobutyl)-3-methyl-2-oxo-2,3-dihydro-1H-imidazo[4,5-c]pyridin-6-yl)amino)-5-(1-methyl-1H-pyrazol-4-yl)phenyl)carbamate

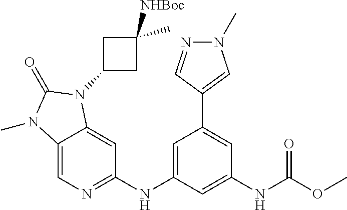

The title compound was prepared according to the procedures described in Example 24, Steps 3-5 with tert-butyl ((1r,3r)-3-amino-1-methylcyclobutyl)carbamate replacing tert-butyl ((1r,3r)-3-aminocyclobutyl)carbamate in Step 3. LCMS calculated for C₂₉H₃₇N₈O₅ (M+H)⁺: m/z=577.3; found: 577.3.

### Step 2. Methyl (3-(1-(1r,3r)-3-(methoxycarbonyl)amino)-3-methylcyclobutyl)-3-methyl-2-oxo-2,3-dihydro-1H-imidazo[4,5-c]pyridin-6-yl)amino)-5-(1-methyl-1H-pyrazol-4-yl)phenyl)carbamate

A reaction vial containing methyl (3-((1-((1r,3r)-3-((tert-butoxycarbonyl)amino)-3-methylcyclobutyl)-3-methyl-2-oxo-2,3-dihydro-1H-imidazo[4,5-c]pyridin-6-yl)amino)-5-(1-methyl-1H-pyrazol-4-yl)phenyl)carbamate (15 mg, 0.03 mmol) was treated with a solution of 4:1 DCM/TFA (2 mL) and stirred at r.t. for 4 h. The solvents were evaporated in vacuo and then DCM (0.6 mL) and N-ethyl-N-isopropylpropan-2-amine (27 μL, 0.16 mmol) were added to the reaction vial and cooled to 0° C. and methyl chloroformate (3 μL, 0.04 mmol) was added. The reaction was allowed to stir at 0° C. for 10 min, then quenched with MeOH (2 ml) and solvents were evaporated in vacuo. The crude material was diluted with MeCN and purified by prep-LCMS (XBridge C18 column, eluting with a gradient of acetonitrile/water containing 0.1% TFA, at flow rate of 60 mL/min. LCMS calculated for C₂₋₆H₃₁N₈O₅ (M+H)⁺: m/z=535.2; found: 535.2.

### Example 30. Methyl (R)-(3-((1-(3,3-difluorocyclopentyl)-3-methyl-2-oxo-2,3-dihydro-1H-imidazo[4,5-c]pyridin-6-yl)amino)-5-(1-methyl-1H-pyrazol-4-yl)phenyl)carbamate

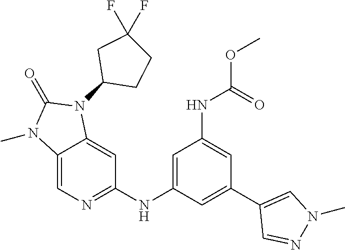

### Step 1. 6-Chloro-1-((1R,3R)-3-hydroxycyclopentyl)-3-methyl-1,3-dihydro-2H-imidazo[4,5-c]pyridin-2-one

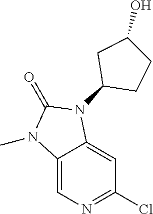 [SMILES: Cn1c(=O)n([C@@H]2CC[C@@H](O)C2)c2cc(Cl)ncc21]

The title compound was prepared according to the procedures described in Example 24, Steps 3-4 with (1R,3R)-3-aminocyclopentan-1-ol hydrochloride replacing tert-butyl ((1r,3r)-3-aminocyclobutyl)carbamate in Step 3. LCMS calculated for C₁₂H₁₅ClN₃O₂ (M+H)⁺: m/z=268.1; found: 268.1.

### Step 2. (R)-6-Chloro-3-methyl-1-(3-oxocyclopentyl)-1,3-dihydro-2H-imidazo[4,5-c]pyridin-2-one

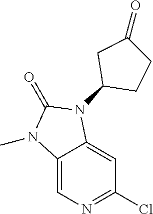 [SMILES: Cn1c(=O)n([C@@H]2CCC(=O)C2)c2cc(Cl)ncc21]

To a reaction vial containing 6-chloro-1-((1R,3R)-3-hydroxycyclopentyl)-3-methyl-1,3-dihydro-2H-imidazo[4,5-c]pyridin-2-one (10 mg, 0.4 mmol) in DCM (0.4 mL) was added Dess-Martin periodinane (24 mg, 0.06 mmol). The reaction was allowed to stir for 16 h at r.t. and upon completion was quenched with a saturated aqueous solution of sodium thiosulfate (1 mL). The reaction mixture was diluted with DCM and the organic layer was subsequently washed with brine, dried over sodium sulfate, filtered, and the solvents were evaporated in vacuo. The crude material was carried into the next reaction without further purification. LCMS calculated for C₁₂H₁₃ClN₃O₂ (M+H)⁺: m/z=266.1; found 266.1

### Step 3. (R)-6-Chloro-1-(3,3-difluorocyclopentyl)-3-methyl-1,3-dihydro-2H-imidazo[4,5-c]pyridin-2-one

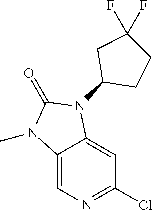 [SMILES: Cn1c(=O)n([C@@H]2CCC(F)(F)C2)c2cc(Cl)ncc21]

To a reaction vial containing (R)-6-chloro-3-methyl-1-(3-oxocyclopentyl)-1,3-dihydro-2H-imidazo[4,5-c]pyridin-2-one (10 mg, 0.04 mmol) was added DCM (0.4 mL) and the reaction mixture was cooled to −78° C. Bis(2-methoxyethyl)aminosulfur trifluoride (151 μL, 0.15 mmol) was added to the reaction mixture which was then allowed to warm to r.t. and stirred at r.t. for 1 h. The reaction mixture was diluted with DCM and the organic layer was subsequently washed with brine, dried over sodium sulfate, filtered, and the solvents were evaporated in vacuo. The crude material was purified by Biotage Isolera. LCMS calculated for C₁₂H₁₃ClF₂N₃O (M+H)⁺: m/z=288.1; found 288.1.

### Step 4. Methyl (R)-(3-((1-(3,3-difluorocyclopentyl)-3-methyl-2-oxo-2,3-dihydro-1H-imidazo[4,5-c]pyridin-6-yl)amino)-5-(1-methyl-1H-pyrazol-4-yl)phenyl)carbamate

To a reaction vial was added (R)-6-chloro-1-(3,3-difluorocyclopentyl)-3-methyl-1,3-dihydro-2H-imidazo[4,5-c]pyridin-2-one (10 mg, 0.035 mmol), methyl (3-amino-5-(1-methyl-1H-pyrazol-4-yl)phenyl)carbamate (9 mg, 0.035 mmol, Example 24, Step 2), XantPhos Pd G3 (3 mg, 3.5 μmol), and cesium carbonate (23 mg, 0.07 mmol). This was evacuated and backfilled with nitrogen 3 times. Subsequently 1,4-dioxane (0.4 mL) was added. The vial was sealed and heated to 100° C. for 16 h. Upon completion, the reaction was diluted with DCM, filtered through Celite and concentrated in vacuo. The crude material was diluted with MeCN and purified by prep-LCMS (XBridge C18 column, eluting with a gradient of acetonitrile/water containing 0.1% TFA, at flow rate of 60 mL/min. LCMS calculated for C₂₄H₂₆F₂N₇O₃ (M+H)⁺: m/z=498.2; found: 498.2. ¹H NMR (400 MHZ, DMSO) δ 9.73 (d, 1H), 9.64 (s, 1H), 8.05-7.98 (m, 2H), 7.74-7.69 (m, 1H), 7.43 (dt, J=9.9, 2.1 Hz, 1H), 7.32 (d, J=5.0 Hz, 1H), 7.25 (s, 1H), 6.98-6.88 (m, 1H), 5.06-4.89 (m, 1H), 3.88 (s, 3H), 3.68 (s, 3H), 3.34 (s, 3H), 2.72-1.99 (m, 6H).

### Example 31. Methyl (3-((1-((1R,3S)-3-fluorocyclopentyl)-3-methyl-2-oxo-2,3-dihydro-1H-imidazo[4,5-c]pyridin-6-yl)amino)-5-(1-methyl-1H-pyrazol-4-yl)phenyl)carbamate

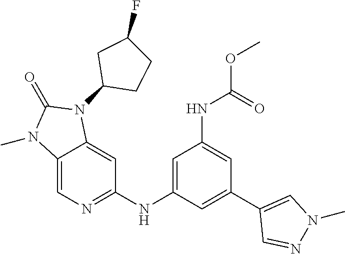

The title compound was prepared according to the procedures described in Example 30, Steps 3-4 with 6-chloro-1-((1R,3R)-3-hydroxycyclopentyl)-3-methyl-1,3-dihydro-2H-imidazo[4,5-c]pyridin-2-one replacing (R)-6-chloro-3-methyl-1-(3-oxocyclopentyl)-1,3-dihydro-2H-imidazo[4,5-c]pyridin-2-one in Step 3. LCMS calculated for C₂₄H₂₇FN₇O₃ (M+H)⁺: m/z=480.2; found: 480.2. ¹H NMR (400 MHZ, DMSO) δ 9.73 (s, 1H), 9.57 (s, 1H), 8.01 (d, J=4.9 Hz, 2H), 7.71 (s, 1H), 7.41 (t, J=2.0 Hz, 1H), 7.32 (s, 1H), 7.26 (s, 1H), 6.90 (s, 1H), 4.96 (h, J=7.9 Hz, 1H), 3.88 (s, 3H), 3.68 (s, 3H), 3.34 (s, 3H), 2.70-2.62 (m, 1H), 2.46-1.87 (m, 6H).

### Example 32. Methyl ((1R,3R)-3-(3-methyl-6-((6-(1-methyl-1H-pyrazol-4-yl)quinolin-2-yl)amino)-2-oxo-2,3-dihydro-1H-imidazo[4,5-c]pyridin-1-yl)cyclopentyl)carbamate

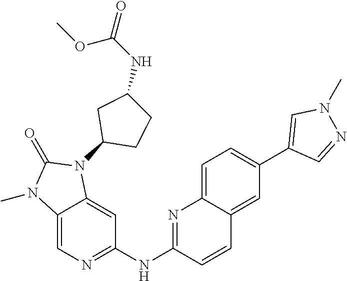

### Step 1. 6-(1-Methyl-1H-pyrazol-4-yl)quinolin-2-amine

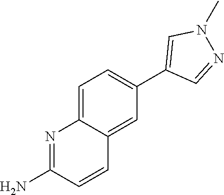 [SMILES: Cn1cc(-c2ccc3nc(N)ccc3c2)cn1]

A reaction vial containing 6-bromoquinolin-2-amine (12 mg, 0.09 mmol), (1-methyl-1H-pyrazol-4-yl)boronic acid (12 mg, 0.094 mmol), potassium phosphate (38 mg, 0.18 mmol) and [1,1′-bis(diphenylphosphino)ferrocene]-dichloropalladium(II) (7 mg, 8.97 μmol) was evacuated and backfilled with nitrogen. Acetonitrile (1.0 mL) and water (0.1 mL) were added to the reaction mixture, which was then stirred at 100° C. for 2 h. The reaction mixture was cooled to r.t and was diluted with EtOAc. The organic layer was subsequently washed with water and brine, dried over sodium sulfate, filtered, and the solvents were evaporated in vacuo. The crude material was purified by Biotage Isolera. LCMS calculated for C₁₃H₁₃N₄ (M+H)⁺: m/z=225.1; found 225.1.

### Step 2. Methyl (1R,3R)-3-(3-methyl-6-((6-(1-methyl-1H-pyrazol-4-yl)quinolin-2-yl)amino)-2-oxo-2,3-dihydro-1H-imidazo[4,5-c]pyridin-1-yl)cyclopentyl)carbamate

To a reaction vial was added 6-(1-methyl-1H-pyrazol-4-yl)quinolin-2-amine (10 mg, 0.03 mmol), methyl ((1R,3R)-3-(6-chloro-3-methyl-2-oxo-2,3-dihydro-1H-imidazo[4,5-c]pyridin-1-yl)cyclopentyl)carbamate (10 mg, 0.05 mmol, common intermediate A), XantPhos Pd G3 (3 mg, 3.1 μmol), and cesium carbonate (30 mg, 0.09 mmol). This was evacuated and backfilled with nitrogen 3 times. Subsequently 1,4-dioxane (0.4 mL) was added. The vial was sealed and heated to 100° C. for 16 h. Upon completion, the reaction was diluted with DCM, filtered through Celite and concentrated in vacuo. The crude material was diluted with MeCN and purified by prep-LCMS (XBridge C18 column, eluting with a gradient of acetonitrile/water containing 0.1% TFA, at flow rate of 60 mL/min. LCMS calculated for C₂₇H₂₉N₈O₃ (M+H)⁺: m/z=513.2; found: 513.2. ¹H NMR (400 MHZ, DMSO) δ 11.85 (s, 1H), 8.29 (s, 1H), 8.14 (s, 2H), 8.07 (s, 2H), 8.01 (s, 2H), 7.82-7.70 (m, 1H), 7.43 (s, 2H), 7.36 (d, J=9.6 Hz, 1H), 7.14 (q, J=7.9 Hz, 1H), 5.00 (p, J=8.6 Hz, 2H), 4.30-4.24 (m, 2H), 3.91 (s, 3H), 3.56 (s, 3H), 2.32-2.23 (m, 1H), 2.19-2.13 (m, 1H), 2.11-2.05 (m, 1H), 2.05-1.92 (m, 1H), 1.71-1.67 (m, 1H).

### Example 33. Methyl ((1R,3R)-3-(3-methyl-6-((8-(1-methyl-1H-pyrazol-4-yl)quinolin-2-yl)amino)-2-oxo-2,3-dihydro-1H-imidazo[4,5-c]pyridin-1-yl)cyclopentyl)carbamate

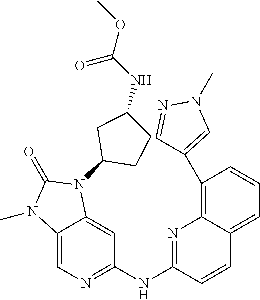 [SMILES: COC(=O)N[C@@H]1CC[C@@H](n2c(=O)n(C)c3cnc(Nc4ccc5cccc(-c6cnn(C)c6)c5n4)cc32)C1]

### Step 1. 8-(1-Methyl-1H-pyrazol-4-yl)quinolin-2-amine

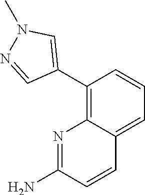 [SMILES: Cn1cc(-c2cccc3ccc(N)nc23)cn1]

The title compound was prepared according to the procedures described in Example 32, Step 1 with 8-bromoquinolin-2-amine replacing 6-bromoquinolin-2-amine. LCMS calculated for C₁₃H₁₃N₄ (M+H)⁺: m/z=225.1; found: 225.1.

### Step 2. Methyl ((1R,3R)-3-(3-methyl-6-((8-(1-methyl-1H-pyrazol-4-yl)quinolin-2-yl)amino)-2-oxo-2,3-dihydro-1H-imidazo[4,5-c]pyridin-1-yl)cyclopentyl)carbamate

The title compound was prepared according to the procedures described in Example 32, Step 2 with 8-(1-methyl-1H-pyrazol-4-yl)quinolin-2-amine replacing 6-(1-methyl-1H-pyrazol-4-yl)quinolin-2-amine. LCMS calculated for C₂₇H₂₉N₈O₃(M+H)⁺: m/z=513.2; found: 513.2. ¹H NMR (400 MHZ, DMSO) δ 12.11 (s, 1H), 8.58 (d, J=9.3 Hz, 1H), 8.30 (s, 1H), 8.07 (s, 1H), 7.96 (d, J=8.0 Hz, 1H), 7.86 (d, J=1.4 Hz, 1H), 7.79-7.58 (m, 1H), 7.57-7.43 (m, 2H), 7.39 (s, 1H), 7.23 (s, 1H), 5.00-4.89 (m, 1H), 4.27-4.17 (m, 1H), 4.07 (s, 3H), 3.55 (s, 3H), 3.41 (s, 3H), 2.32-2.07 (m, 3H), 2.07-1.87 (m, 2H), 1.66-1.62 (m, 1H).

### Example 34. Methyl ((1R,3R)-3-(3-methyl-6-((3-(1-methyl-1H-pyrazol-4-yl)imidazo[1,2-b]pyridazin-6-yl)amino)-2-oxo-2,3-dihydro-1H-imidazo[4,5-c]pyridin-1-yl)cyclopentyl)carbamate

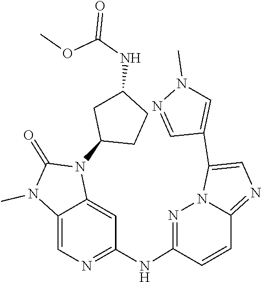 [SMILES: COC(=O)N[C@@H]1CC[C@@H](n2c(=O)n(C)c3cnc(Nc4ccc5ncc(-c6cnn(C)c6)n5n4)cc32)C1]

### Step 1. 3-(1-Methyl-1H-pyrazol-4-yl)imidazo[1,2-b]pyridazin-6-amine

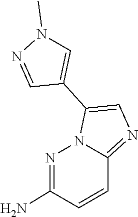 [SMILES: Cn1cc(-c2cnc3ccc(N)nn23)cn1]

The title compound was prepared according to the procedures described in Example 32, Step 1 with 3-bromoimidazo[1,2-b]pyridazin-6-amine replacing 6-bromoquinolin-2-amine. LCMS calculated for C₁₀H₁₁N₆ (M+H)⁺: m/z=215.1; found: 215.1.

### Step 2. Methyl ((1R,3R)-3-(3-methyl-6-((3-(1-methyl-1H-pyrazol-4-yl)imidazo[1,2-b]pyridazin-6-yl)amino)-2-oxo-2,3-dihydro-1H-imidazo[4,5-c]pyridin-1-yl)cyclopentyl)carbamate

The title compound was prepared according to the procedures described in Example 32, Step 2 with 3-(1-methyl-1H-pyrazol-4-yl)imidazo[1,2-b]pyridazin-6-amine replacing 6-(1-methyl-1H-pyrazol-4-yl)quinolin-2-amine. LCMS calculated for C₂₄H₂₇N₁₀O₃ (M+H)⁺: m/z=503.2; found: 503.2.

### Example 35. Methyl ((1R,3R)-3-(3-methyl-6-((2-(1-methyl-1H-pyrazol-4-yl)pyrazolo[1,5-a]pyrimidin-5-yl)amino)-2-oxo-2,3-dihydro-1H-imidazo[4,5-c]pyridin-1-yl)cyclopentyl)carbamate

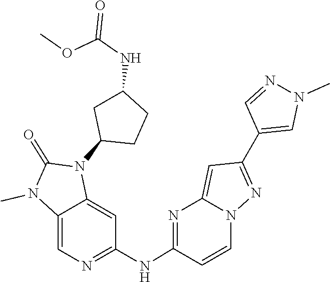

### Step 1. 2-Bromopyrazolo[1,5-a]pyrimidin-5-amine

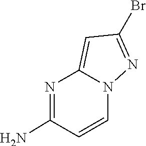 [SMILES: Nc1ccn2nc(Br)cc2n1]

A microwave vessel containing a mixture of 2-bromo-5-chloropyrazolo[1,5-a]pyrimidine (233 mg, 1.0 mmol), ammonium hydroxide solution (3.9 mL, 30.1 mmol), and ethanol (6.7 mL) was irradiated at 100° C. for 1 h using a Biotage Initator+Microwave Synthesizer. After cooling to room temperature, the reaction mixture was extracted into CH₂C₁₂. The organic phase was then washed with water (2×), brine, then dried over MgSO₄, filtered, and concentrated in vacuo. The resulting crude material was taken to the next step reaction without further purification. LCMS calculated for C₆H₆BrN₄ (M+H)⁺: m/z=213.0; found 213.0.

### Step 2. Methyl ((1R,3R)-3-(3-methyl-6-((2-(1-methyl-1H-pyrazol-4-yl)pyrazolo[1,5-a]pyrimidin-5-yl)amino)-2-oxo-2,3-dihydro-1H-imidazo[4,5-c]pyridin-1-yl)cyclopentyl)carbamate

The title compound was prepared according to the procedures described in Example 32, Steps 1-2 with 2-bromopyrazolo[1,5-a]pyrimidin-5-amine replacing 6-bromoquinolin-2-amine in Step 1. LCMS calculated for C₂₄H₂₇N1003 (M+H)⁺: m/z=503.2; found: 503.2. ¹H NMR (400 MHz, DMSO) δ 10.60 (s, 1H), 8.74 (d, J=7.5 Hz, 1H), 8.21 (s, 1H), 8.18 (d, J=6.9 Hz, 2H), 7.90 (s, 1H), 7.45 (d, J=7.2 Hz, 1H), 6.75 (d, J=7.5 Hz, 1H), 6.61 (s, 1H), 5.01 (p, J=8.8 Hz, 1H), 4.41-4.35 (m, 1H), 3.90 (s, 3H), 3.59 (s, 3H), 3.38 (s, 3H), 2.35 (dd, J=13.7, 7.1 Hz, 2H), 2.16-1.94 (m, 3H), 1.69 (m, 1H).

### Example 36. Methyl ((1R,3R)-3-(3-methyl-6-((3-(1-methyl-1H-pyrazol-4-yl)pyrazolo[1,5-a]pyrimidin-5-yl)amino)-2-oxo-2,3-dihydro-1H-imidazo[4,5-c]pyridin-1-yl)cyclopentyl)carbamate

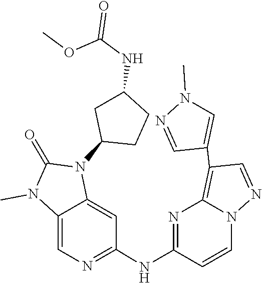 [SMILES: COC(=O)N[C@@H]1CC[C@@H](n2c(=O)n(C)c3cnc(Nc4ccn5ncc(-c6cnn(C)c6)c5n4)cc32)C1]

### Step 1. 3-Bromopyrazolo[1,5-a]pyrimidin-5-amine

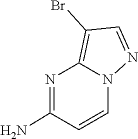 [SMILES: Nc1ccn2ncc(Br)c2n1]

The title compound was prepared according to the procedures described in Example 35, Step 1 with 3-bromo-5-chloropyrazolo[1,5-a]pyrimidine replacing 2-bromo-5-chloropyrazolo[1,5-a]pyrimidine. LCMS calculated for C₆H₆BrN₄ (M+H)⁺: m/z=213.0; found 213.0.

### Step 2. Methyl ((1R,3R)-3-(3-methyl-6-((3-(1-methyl-1H-pyrazol-4-yl)pyrazolo[1,5-a]pyrimidin-5-yl)amino)-2-oxo-2,3-dihydro-1H-imidazo[4,5-c]pyridin-1-yl)cyclopentyl)carbamate

The title compound was prepared according to the procedures described in Example 32, Steps 1-2 with 3-bromopyrazolo[1,5-a]pyrimidin-5-amine replacing 6-bromoquinolin-2-amine in Step 1. LCMS calculated for C₂₄H₂₇N₁₀O₃ (M+H)⁺: m/z=503.2; found: 503.2.

### Example 37. Methyl ((1R,3R)-3-(3-methyl-2-oxo-6-((6-((tetrahydro-2H-pyran-4-yl)oxy)pyridin-2-yl)amino)-2,3-dihydro-1H-imidazo[4,5-c]pyridin-1-yl)cyclopentyl)carbamate

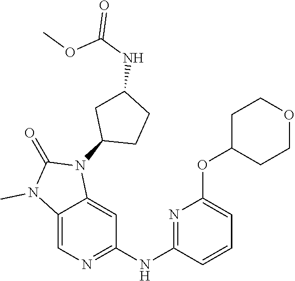 [SMILES: COC(=O)N[C@@H]1CC[C@@H](n2c(=O)n(C)c3cnc(Nc4cccc(OC5CCOCC5)n4)cc32)C1]

### Step 1. 6-((Tetrahydro-2H-pyran-4-yl)oxy)pyridin-2-amine

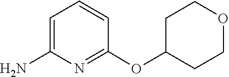 [SMILES: Nc1cccc(OC2CCOCC2)n1]

The title compound was prepared according to the procedures described in Example 23, with tetrahydro-2H-pyran-4-ol replacing quinolin-6-ol in Step 1. LCMS calculated for C₁₀H₁₅N₂O₂ (M+H)⁺: m/z=195.1; found 195.1.

### Step 2. Methyl ((1R,3R)-3-(3-methyl-2-oxo-6-((6-((tetrahydro-2H-pyran-4-yl)oxy)pyridin-2-yl)amino)-2,3-dihydro-1H-imidazo[4,5-c]pyridin-1-yl)cyclopentyl)carbamate

The title compound was prepared according to the procedures described in Example 23, with 6-((tetrahydro-2H-pyran-4-yl)oxy)pyridin-2-amine replacing 6-(quinolin-6-yloxy)pyridin-2-amine in Step 2. LCMS calculated for C₂₄H₃₁N₆O₅ (M+H)⁺: m/z=483.2; found 483.2.

### Example 38. Methyl ((1R,3R)-3-(3-methyl-6-((3-(1-methyl-1H-imidazol-5-yl)imidazo[1,2-b]pyridazin-6-yl)amino)-2-oxo-2,3-dihydro-1H-imidazo[4,5-c]pyridin-1-yl)cyclopentyl)carbamate

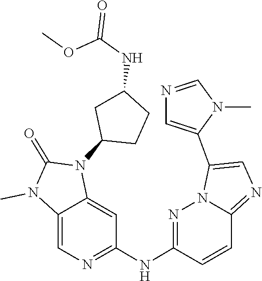 [SMILES: COC(=O)N[C@@H]1CC[C@@H](n2c(=O)n(C)c3cnc(Nc4ccc5ncc(-c6cncn6C)n5n4)cc32)C1]

### Step 1. 3-(1-Methyl-1H-imidazol-5-yl)imidazo[1,2-b]pyridazin-6-amine

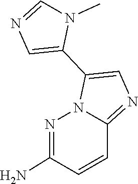 [SMILES: Cn1cncc1-c1cnc2ccc(N)nn12]

The title compound was prepared according to the procedures described in Example 32, Step 1 with 3-bromoimidazo[1,2-b]pyridazin-6-amine replacing 6-bromoquinolin-2-amine and with (1-methyl-1H-imidazol-5-yl)boronic acid replacing (1-methyl-1H-pyrazol-4-yl)boronic acid. LCMS calculated for C₁₀H₁₁N₆ (M+H)⁺: m/z=215.1; found: 215.1.

### Step 2. Methyl ((1R,3R)-3-(3-methyl-6-((3-(1-methyl-1H-imidazol-5-yl)imidazo[1,2-b]pyridazin-6-yl)amino)-2-oxo-2,3-dihydro-1H-imidazo[4,5-c]pyridin-1-yl)cyclopentyl)carbamate

The title compound was prepared according to the procedures described in Example 32, Step 2 with 3-(1-methyl-1H-imidazol-5-yl)imidazo[1,2-b]pyridazin-6-amine replacing 6-(1-methyl-1H-pyrazol-4-yl)quinolin-2-amine. LCMS calculated for C₂₄H₂₇N₁₀O₃ (M+H)⁺: m/z=503.2; found: 503.2.

### Example 39. Methyl (1R,5S)-6-(6-((3-((methoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)-3-azabicyclo[3.1.0]hexane-3-carboxylate

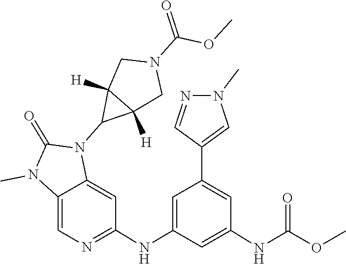

### Step 1. tert-Butyl (1R,5S,6s)-6-((2-chloro-5-nitropyridin-4-yl)amino)-3-azabicyclo[3.1.0]hexane-3-carboxylate

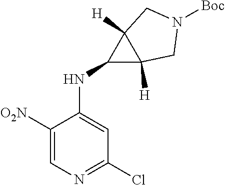 [SMILES: CC(C)(C)OC(=O)N1C[C@@H]2[C@H](C1)[C@H]2Nc1cc(Cl)ncc1[N+](=O)[O-]]

To a reaction vial containing 2,4-dichloro-5-nitropyridine (511 mg, 2.65 mmol) in THF (10 mL) was added N,N-diisopropylethylamine (529 μL, 3.03 mmol) and tert-butyl (1R,5S,6s)-6-amino-3-azabicyclo[3.1.0]hexane-3-carboxylate (500 mg, 2.52 mmol). The reaction mixture was stirred at 55° C. for 1 h. The reaction mixture was allowed to cool to r.t. before washed with water. The mixture was extracted with EtOAc and the organic layer was subsequently washed with water and brine, dried over sodium sulfate, filtered, and the solvents were evaporated in vacuo. The crude material was used directly for next step. LCMS calculated for C₁₅H₂₀ClN₄O₄ (M+H)⁺: m/z=355.1; found 355.2.

### Step 2. tert-Butyl (1R,5S,6s)-6-((5-amino-2-chloropyridin-4-yl)amino)-3-azabicyclo[3.1.0]hexane-3-carboxylate

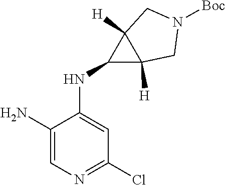 [SMILES: CC(C)(C)OC(=O)N1C[C@@H]2[C@H](C1)[C@H]2Nc1cc(Cl)ncc1N]

To a reaction vial containing tert-butyl (1R,5S,6s)-6-((2-chloro-5-nitropyridin-4-yl)amino)-3-azabicyclo[3.1.0]hexane-3-carboxylate (895 mg, 2.52 mmol) in THF (10 mL) and water (2 mL) was added zinc dust (820 mg, 12.61 mmol) and ammonium chloride (668 mg, 12.61 mmol). The reaction mixture was stirred at 40° C. for 30 min. The reaction mixture was allowed to cool to r.t. before filtered through celite. The mixture was extracted with EtOAc and the organic layer was subsequently washed with water and brine, dried over sodium sulfate, filtered, and the solvents were evaporated in vacuo. The crude material was used directly for next step. LCMS calculated for C₁₅H₂₂ClN₄O₂ (M+H)⁺: m/z=325.1; found 325.2.

### Step 3. tert-Butyl (1R,5S,6r)-6-(6-chloro-2-oxo-2,3-dihydro-1H-imidazo[4,5-c]pyridin-1-yl)-3-azabicyclo[3.1.0]hexane-3-carboxylate

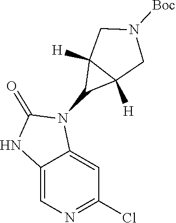

To a reaction vial containing tert-butyl (1R,5S,6s)-6-((5-amino-2-chloropyridin-4-yl)amino)-3-azabicyclo[3.1.0]hexane-3-carboxylate (819 mg, 2.52 mmol) and N,N-diisopropylethylamine (528 μL, 3.03 mmol) in MeCN (20 mL) was added bis(2,5-dioxopyrrolidin-1-yl) carbonate (711 mg, 2.77 mmol) portionwise. The reaction mixture was stirred at r.t. for 2 h. The reaction mixture was diluted with water and extracted with EtOAc. The organic layer was subsequently washed with water and brine, dried over sodium sulfate, filtered, and the solvents were evaporated in vacuo. The crude material was purified by Biotage Isolera to give the product. LCMS calculated for C₁₆H₂₀ClN₄O₃ (M+H)⁺: m/z=351.1; found 351.2.

### Step 4. tert-Butyl (1R,5S,6r)-6-(6-chloro-3-methyl-2-oxo-2,3-dihydro-1H-imidazo[4,5-c]pyridin-1-yl)-3-azabicyclo[3.1.0]hexane-3-carboxylate

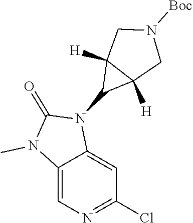

To a reaction vial containing tert-butyl (1R,5S,6r)-6-(6-chloro-2-oxo-2,3-dihydro-1H-imidazo[4,5-c]pyridin-1-yl)-3-azabicyclo[3.1.0]hexane-3-carboxylate (610 mg, 1.74 mmol) and sodium hydride (238 mg, 6.96 mmol, 70% dispersion in mineral oil) in THF (8 mL) was added iodomethane (435 μL, 6.96 mmol) dropwise. The reaction mixture was stirred at r.t. for 3 h. The reaction mixture was quenched with water and extracted with EtOAc and the organic layer was subsequently washed with water and brine, dried over sodium sulfate, filtered, and the solvents were evaporated in vacuo. The crude material was purified by Biotage Isolera to give the product. LCMS calculated for C₁₇H₂₂ClN₄O₃ (M+H)⁺: m/z=365.1; found 365.2.

### Step 5. tert-Butyl (1R,5S)-6-(2-((3-((methoxycarbonyl)amino)-5-(1-methyl-1H-pyrazol-4-yl)phenyl)amino)-7-methyl-8-oxo-7,8-dihydro-9H-purin-9-yl)-3-azabicyclo[3.1.0]hexane-3-carboxylate

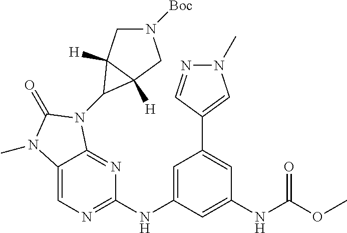

A reaction vial containing tert-butyl (1R,5S,6r)-6-(6-chloro-3-methyl-2-oxo-2,3-dihydro-1H-imidazo[4,5-c]pyridin-1-yl)-3-azabicyclo[3.1.0]hexane-3-carboxylate (158 mg, 0.433 mmol), methyl (3-amino-5-(1-methyl-1H-pyrazol-4-yl)phenyl)carbamate (139 mg, 0.563 mmol, Example 53, Step 3), cesium carbonate (282 mg, 0.866 mmol), tris(dibenzylideneacetone)dipalladium(0) (60 mg, 0.065 mmol) and 4,5-bis(diphenylphosphino)-9,9-dimethylxanthene (75 mg, 0.13 mmol) was evacuated and backfilled with nitrogen. 1,4-Dioxane (4.3 mL) was added to the reaction mixture, which was then stirred at 100° C. for 2 h. The reaction mixture was cooled to r.t and was diluted with EtOAc. The organic layer was subsequently washed with water and brine, dried over sodium sulfate, filtered, and the solvents were evaporated in vacuo. The crude material was purified by Biotage Isolera. LCMS calculated for C₂₈H₃₄N₉O₅ (M+H)⁺: m/z=576.3; found 576.3.

### Step 6. Methyl (3-((9-((1R,5S)-3-azabicyclo[3.1.0]hexan-6-yl)-7-methyl-8-oxo-8,9-dihydro-7H-purin-2-yl)amino)-5-(1-methyl-1H-pyrazol-4-yl)phenyl)carbamate

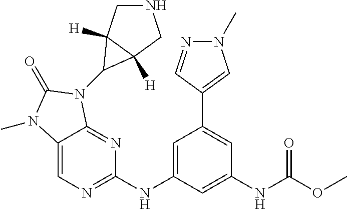

To a reaction vial containing tert-butyl (1R,5S)-6-(2-((3-((methoxycarbonyl)amino)-5-(1-methyl-1H-pyrazol-4-yl)phenyl)amino)-7-methyl-8-oxo-7,8-dihydro-9H-purin-9-yl)-3-azabicyclo[3.1.0]hexane-3-carboxylate (130 mg, 0.226 mmol) was added DCM (1 mL) and trifluoroacetic acid (0.5 mL). The reaction mixture was then stirred at r.t. for 1 h. The reaction mixture was concentrated in vacuo and the crude material was used directly for next step. LCMS calculated for C₂₃H₂₆N₉O₃ (M+H)⁺: m/z=476.2; found 476.2.

### Step 7. Methyl (1R,5S)-6-(6-(3-((methoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)-3-azabicyclo[3.1.0]hexane-3-carboxylate

To a reaction vial containing methyl (3-((9-((1R,5S)-3-azabicyclo[3.1.0]hexan-6-yl)-7-methyl-8-oxo-8,9-dihydro-7H-purin-2-yl)amino)-5-(1-methyl-1H-pyrazol-4-yl)phenyl)carbamate (10 mg, 0.021 mmol) and triethylamine (7.3 μL, 0.053 mmol) in dioxane (0.5 mL) and water (0.1 mL) was added methyl chloroformate (2 μL, 0.025 mmol). The reaction mixture was stirred at r.t. for 20 min, then quenched with MeOH (2 ml) and solvents were evaporated in vacuo. The crude material was diluted with MeCN and purified by prep-LCMS (XBridge C18 column, eluting with a gradient of acetonitrile/water containing 0.1% TFA, at flow rate of 60 mL/min. LCMS calculated for C₂₋₆H₂₉N₈O₅ (M+H)⁺: m/z=533.2; found: 533.2. ¹H NMR (400 MHZ, DMSO-d₆) δ 9.73 (s, 1H), 9.53 (s, 1H), 8.02 (s, 1H), 7.95 (s, 1H), 7.72 (s, 1H), 7.47 (d, J=2.0 Hz, 1H), 7.30 (d, J=13.9 Hz, 2H), 6.91 (s, 1H), 3.88 (s, 3H), 3.72-3.66 (m, 5H), 3.58 (s, 3H), 3.50 (m, 2H), 3.32 (s, 3H), 2.74 (t, J=2.2 Hz, 1H), 2.23 (s, 2H).

### Example 40. Methyl (3-((1-(2-acetyl-2-azabicyclo[2.2.1]heptan-5-yl)-3-methyl-2-oxo-2,3-dihydro-1H-imidazo[4,5-c]pyridin-6-yl)amino)-5-(1-methyl-1H-pyrazol-4-yl)phenyl)carbamate

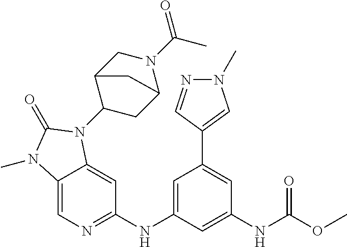

The title compound was prepared according to the procedures described in Example 39, with tert-butyl 5-amino-2-azabicyclo[2.2.1]heptane-2-carboxylate replacing tert-butyl (1R,5S,6s)-6-amino-3-azabicyclo[3.1.0]hexane-3-carboxylate (Step 1) and acetyl chloride replacing methyl chloroformate (Step 7). LCMS calculated for C₂₇H₃₁N₈O₄ (M+H)⁺: m/z=531.2; found: 531.2.

### Example 41. Methyl ((1R,3R)-3-(6-((3-((3,3-difluorocyclobutane)-1-sulfonamido)-5-(tetrahydro-2H-pyran-4-yl)phenyl)amino)-3-methyl-2-oxo-2,3-dihydro-1H-imidazo[4,5-c]pyridin-1-yl)cyclopentyl)carbamate

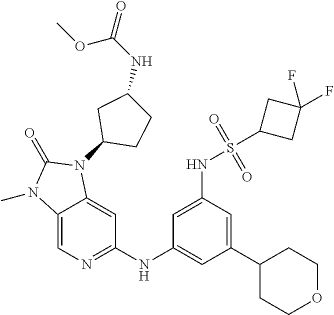 [SMILES: COC(=O)N[C@@H]1CC[C@@H](n2c(=O)n(C)c3cnc(Nc4cc(NS(=O)(=O)C5CC(F)(F)C5)cc(C5CCOCC5)c4)cc32)C1]

To a solution of methyl ((1R,3R)-3-(6-((3-amino-5-(tetrahydro-2H-pyran-4-yl)phenyl)amino)-3-methyl-2-oxo-2,3-dihydro-1H-imidazo[4,5-c]pyridin-1-yl)cyclopentyl)carbamate (10 mg, 0.021 mmol) and DIPEA (7 μl, 0.042 mmol) in dioxane (0.5 ml) was added 3,3-difluorocyclobutane-1-sulfonyl chloride (5 mg, 0.025 mmol). The mixture was stirred at r.t. for 1 h. and concentrated. The residue was purified with prep-LCMS (XBridge C18 column, eluting with a gradient of acetonitrile/water containing 0.1% TFA, at flow rate of 60 mL/min). LCMS calculated for C₂₉H₃₇F₂N₆O₆S (M+H)⁺: m/z=635.2; found 635.2.

### Example 42. Methyl (3-((1-(4-((methoxycarbonyl)amino)-4-methylcyclohexyl)-3-methyl-2-oxo-2,3-dihydro-1H-imidazo[4,5-c]pyridin-6-yl)amino)-5-(1-methyl-1H-pyrazol-4-yl)phenyl)carbamate

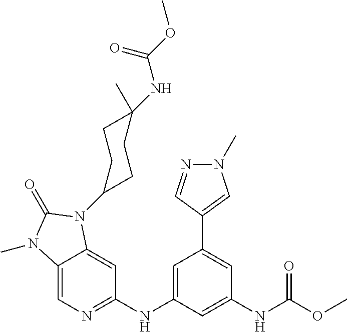

The title compound was prepared according to the procedures described in Example 39, with tert-butyl ((1s,4s)-4-amino-1-methylcyclohexyl)carbamate replacing tert-butyl (1R,5S,6s)-6-amino-3-azabicyclo[3.1.0]hexane-3-carboxylate (Step 1). LCMS calculated for C₂₈H₃₅N₈O₅ (M+H)⁺: m/z=563.3; found: 563.3. ¹H NMR (400 MHZ, DMSO-d₆) δ 9.72 (s, 1H), 9.46 (s, 1H), 8.00 (s, 1H), 7.98 (s, 1H), 7.70 (s, 1H), 7.47 (s, 1H), 7.28 (s, 1H), 6.99 (s, 1H), 6.94 (s, 1H), 4.24-4.14 (m, 2H), 3.88 (s, 3H), 3.69 (s, 3H), 3.50 (s, 3H), 3.34 (s, 3H), 2.14 (dd, J=15.1, 11.5 Hz, 2H), 1.95 (d, J=12.8 Hz, 2H), 1.77-1.62 (m, 4H), 1.33 (s, 3H).

### Example 43. 2-Methoxyethyl ((1R,3R)-3-(6-((3-((cyclobutoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate

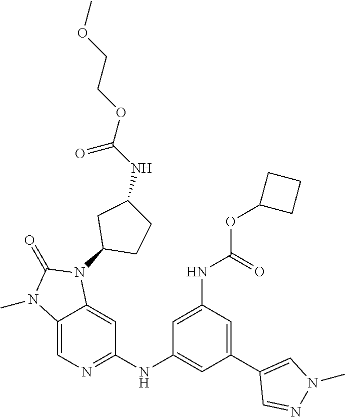

### Step 1. tert-Butyl ((1R,3R)-3-(6-chloro-3-methyl-2-oxo-2,3-dihydro-1H-imidazo[4,5-c]pyridin-1-yl)cyclopentyl)carbamate

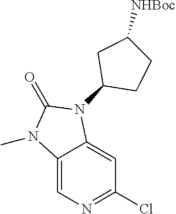 [SMILES: Cn1c(=O)n([C@@H]2CC[C@@H](NC(=O)OC(C)(C)C)C2)c2cc(Cl)ncc21]

To a vial containing tert-butyl ((1R,3R)-3-(6-chloro-2-oxo-2,3-dihydro-1H-imidazo[4,5-c]pyridin-1-yl)cyclopentyl)carbamate (2.60 g, 7.37 mmol, Intermediate A, Step 3) and cesium carbonate (4.80 g, 14.74 mmol) in THF (24.6 mL) was added iodomethane (2.61 g, 1.15 mL, 18.42 mmol). After stirring at r.t. for 6 h, the reaction mixture was diluted with DCM (50 mL), filtered through a celite pad and washed with DCM (15 mL) for 3 times. The filtrate was concentrated in vacuo and was washed with aqueous saturated sodium bicarbonate solution. The organic phase was washed with brine, dried over MgSO₄ and concentrated in vacuo. The crude material was used directly for next step. LCMS calculated for C₁₇H₂₄ClN₄O₃ (M+H)⁺: m/z=367.1; found 367.1.

### Step 2. Cyclobutyl (3-((1-((1R,3R)-3-((tert-butoxycarbonyl)amino)cyclopentyl)-3-methyl-2-oxo-2,3-dihydro-1H-imidazo[4,5-c]pyridin-6-yl)amino)-5-(1-methyl-1H-pyrazol-4-yl)phenyl)carbamate

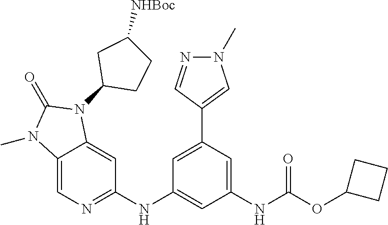 [SMILES: Cn1cc(-c2cc(NC(=O)OC3CCC3)cc(Nc3cc4c(cn3)n(C)c(=O)n4[C@@H]3CC[C@@H](NC(=O)OC(C)(C)C)C3)c2)cn1]

A reaction vial containing tert-butyl ((1R,3R)-3-(6-chloro-3-methyl-2-oxo-2,3-dihydro-1H-imidazo[4,5-c]pyridin-1-yl)cyclopentyl)carbamate (600 mg, 1.64 mmol), cyclobutyl (3-amino-5-(1-methyl-1H-pyrazol-4-yl)phenyl)carbamate (562 mg, 1.96 mmol, Example 56, Step 3), cesium carbonate (1.07 g, 3.27 mmol), tris(dibenzylideneacetone)dipalladium(0) (225 mg, 0.245 mmol) and 4,5-bis(diphenylphosphino)-9,9-dimethylxanthene (284 mg, 0.491 mmol) was evacuated and backfilled with nitrogen. 1,4-Dioxane (8 mL) was added to the reaction mixture, which was then stirred at 100° C. for 2 h. The reaction mixture was cooled to r.t and was diluted with EtOAc. The organic layer was subsequently washed with water and brine, dried over sodium sulfate, filtered, and the solvents were evaporated in vacuo. The crude material was purified by Biotage Isolera. LCMS calculated for C₃₂H₄₁N₈O₅ (M+H)⁺: m/z=617.3; found 617.3.

### Step 3. Cyclobutyl (3-((1-((1R,3R)-3-aminocyclopentyl)-3-methyl-2-oxo-2,3-dihydro-1H-imidazo[4,5-c]pyridin-6-yl)amino)-5-(1-methyl-1H-pyrazol-4-yl)phenyl) carbamate

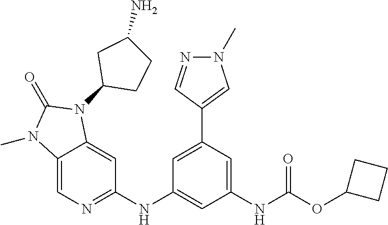 [SMILES: Cn1cc(-c2cc(NC(=O)OC3CCC3)cc(Nc3cc4c(cn3)n(C)c(=O)n4[C@@H]3CC[C@@H](N)C3)c2)cn1]

To a reaction vial containing cyclobutyl (3-((1-((1R,3R)-3-((tert-butoxycarbonyl)amino)-cyclopentyl)-3-methyl-2-oxo-2,3-dihydro-1H-imidazo[4,5-c]pyridin-6-yl)amino)-5-(1-methyl-1H-pyrazol-4-yl)phenyl)carbamate (960 mg, 1.56 mmol) was added HCl in dioxane (4 mL, 16 mmol, 4M). The reaction mixture was then stirred at r.t. for 1 h. The reaction mixture was concentrated in vacuo and the crude material was used directly for next step. LCMS calculated for C₂₇H₃₃N₈O₃(M+H)⁺: m/z=517.3; found 517.3.

### Step 4. 2-Methoxyethyl ((1R,3R)-3-(6-((3-((cyclobutoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate

To a reaction vial containing cyclobutyl (3-((1-((1R,3R)-3-aminocyclopentyl)-3-methyl-2-oxo-2,3-dihydro-1H-imidazo[4,5-c]pyridin-6-yl)amino)-5-(1-methyl-1H-pyrazol-4-yl)phenyl)carbamate (20 mg, 0.036 mmol) and triethylamine (20 μL, 0.145 mmol) in dioxane (0.5 mL) was added 2-methoxyethyl carbonochloridate (2 μL, 0.018 mmol). The reaction mixture was stirred at r.t. for 10 min, then quenched with MeOH (2 ml) and solvents were evaporated in vacuo. The crude material was diluted with MeCN and purified by prep-LCMS (XBridge C18 column, eluting with a gradient of acetonitrile/water containing 0.1% TFA, at flow rate of 60 mL/min. LCMS calculated for C₃₁H₃₉N₈O₆ (M+H)⁺: m/z=619.3; found: 619.3. ¹H NMR (400 MHZ, DMSO-d₆) δ 9.70 (s, 1H), 9.48 (s, 1H), 7.99 (s, 1H), 7.96 (s, 1H), 7.69 (s, 1H), 7.44 (d, J=7.0 Hz, 1H), 7.39 (s, 1H), 7.30 (s, 2H), 6.83 (s, 1H), 5.00-4.85 (m, 2H), 4.19-4.13 (m, 1H), 4.06 (t, J=4.7 Hz, 2H), 3.88 (s, 3H), 3.53-3.46 (m, 2H), 3.33 (s, 3H), 3.26 (s, 3H), 2.38-2.19 (m, 3H), 2.16-1.97 (m, 5H), 1.89 (ddd, J=13.8, 9.3, 4.9 Hz, 1H), 1.77 (q, J=10.2 Hz, 1H), 1.70-1.56 (m, 2H).

### Example 44. Methyl ((1R,3R)-3-(6-((3-((cyclobutoxycarbonyl)amino)-5-(tetrahydro-2H-pyran-4-yl)phenyl)amino)-3-methyl-2-oxo-2,3-dihydro-1H-imidazo[4,5-c]pyridin-1-yl)cyclopentyl)carbamate

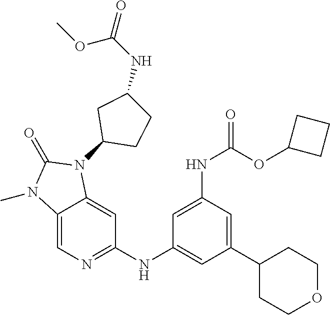 [SMILES: COC(=O)N[C@@H]1CC[C@@H](n2c(=O)n(C)c3cnc(Nc4cc(NC(=O)OC5CCC5)cc(C5CCOCC5)c4)cc32)C1]

The title compound was prepared according to the procedure described in Example 41, using cyclobutyl carbonochloridate instead of 3,3-difluorocyclobutane-1-sulfonyl chloride. LCMS calculated for C₃₀H₃₉N₆O₆ (M+H)⁺: m/z=579.3; found 579.3

### Example 45. Cyclobutyl ((1R,3R)-3-(6-((3-((cyclobutoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate

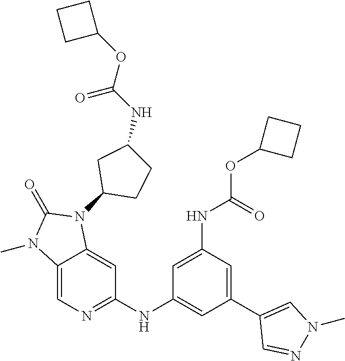

The title compound was prepared according to the procedures described in Example 43, with cyclobutyl carbonochloridate replacing 2-methoxyethyl carbonochloridate (Step 4). LCMS calculated for C₃₂H₃₉N₈O₅ (M+H)⁺: m/z=615.3; found: 615.3. ¹H NMR (400 MHZ, DMSO-d₆) δ 9.74 (s, 1H), 9.65 (s, 1H), 8.01 (s, 1H), 7.97 (s, 1H), 7.71 (s, 1H), 7.40-7.29 (m, 3H), 7.24 (s, 1H), 6.86 (s, 1H), 4.92 (m, 2H), 4.81 (p, J=7.5 Hz, 1H), 4.13 (d, J=6.5 Hz, 1H), 3.88 (s, 3H), 3.33 (s, 3H), 2.36-2.27 (m, 2H), 2.22 (m, 3H), 2.08 (m, 4H), 2.03-1.83 (m, 4H), 1.82-1.64 (m, 2H), 1.64-1.50 (m, 3H).

### Example 46. Ethyl ((1R,3R)-3-(6-((3-((cyclobutoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate

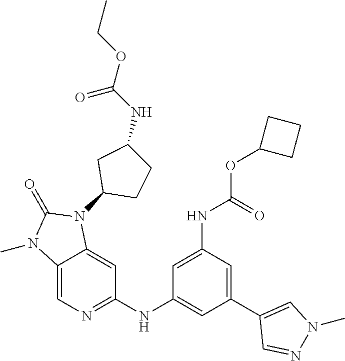

The title compound was prepared according to the procedures described in Example 43, with ethyl carbonochloridate replacing 2-methoxyethyl carbonochloridate (Step 4). LCMS calculated for C₃₀H₃₇N₈O₅ (M+H)⁺: m/z=589.3; found: 589.3. ¹H NMR (400 MHZ, DMSO-d₆) δ 9.71 (s, 1H), 9.53 (s, 1H), 8.00 (s, 1H), 7.97 (s, 1H), 7.70 (s, 1H), 7.39 (d, J=2.1 Hz, 1H), 7.30 (d, J=13.3 Hz, 3H), 6.84 (s, 1H), 4.91 (m, 2H), 4.16 (d, J=7.4 Hz, 1H), 3.99 (q, J=7.1 Hz, 2H), 3.88 (s, 3H), 3.33 (s, 3H), 2.38-2.19 (m, 3H), 2.15-1.94 (m, 5H), 1.89 (m, 1H), 1.77 (q, J=10.4 Hz, 1H), 1.61 (m, 2H), 1.17 (t, J=7.1 Hz, 3H).

### Example 47. Prop-2-yn-1-yl ((1R,3R)-3-(6-((3-((cyclobutoxycarbonyl)amino)-5-(1-methyl-1H-pyrazol-4-yl)phenyl)amino)-3-methyl-2-oxo-2,3-dihydro-1H-imidazo[4,5-c)pyridin-1-yl)cyclopentyl)carbamate

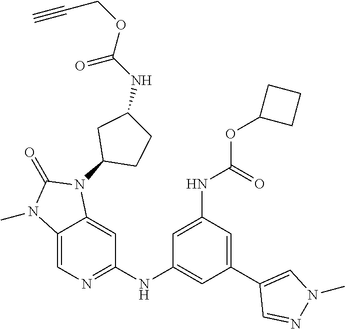

The title compound was prepared according to the procedures described in Example 43, with prop-2-yn-1-yl carbonochloridate replacing 2-methoxyethyl carbonochloridate (Step 4). LCMS calculated for C₃₁H₃₅N₈O₅ (M+H)⁺: m/z=599.3; found: 599.3. ¹H NMR (400 MHz, DMSO-d₆) δ 9.72 (s, 1H), 9.58 (s, 1H), 8.00 (s, 1H), 7.97 (s, 1H), 7.70 (s, 1H), 7.58 (d, J=7.1 Hz, 1H), 7.38 (t, J=2.0 Hz, 1H), 7.34 (s, 1H), 7.26 (s, 1H), 6.86 (s, 1H), 4.94 (p, J=8.4, 7.9 Hz, 2H), 4.62 (d, J=2.4 Hz, 2H), 4.17 (d, J=6.9 Hz, 1H), 3.88 (s, 3H), 3.48 (t, J=2.4 Hz, 1H), 3.34 (s, 3H), 2.36-2.21 (m, 3H), 2.17-1.95 (m, 5H), 1.90 (ddd, J=13.9, 9.5, 5.0 Hz, 1H), 1.77 (q, J=10.2 Hz, 1H), 1.70-1.53 (m, 2H).

### Example 48. Tetrahydro-2H-pyran-4-yl ((1R,3R)-3-(6-((3-((cyclobutoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate

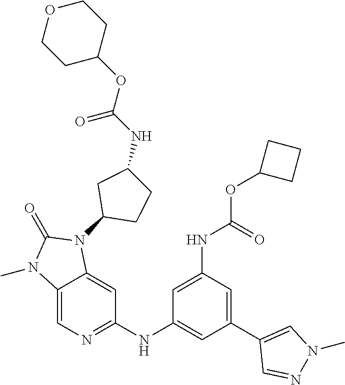

The title compound was prepared according to the procedures described in Example 43, with tetrahydro-2H-pyran-4-yl carbonochloridate replacing 2-methoxyethyl carbonochloridate (Step 4). LCMS calculated for C₃₃H₄₁N₈O₆ (M+H)⁺: m/z=645.3; found: 645.3. ¹H NMR (400 MHZ, DMSO-d₆) δ 9.72 (s, 1H), 9.59 (s, 1H), 8.00 (s, 1H), 7.97 (s, 1H), 7.70 (s, 1H), 7.41-7.31 (m, 3H), 7.26 (s, 1H), 6.86 (s, 1H), 5.00-4.85 (m, 2H), 4.69 (dd, J=9.4, 4.9 Hz, 1H), 4.17 (d, J=6.7 Hz, 1H), 3.88 (s, 3H), 3.81 (d, J=11.5 Hz, 2H), 3.42 (t, J=10.1 Hz, 2H), 3.33 (s, 3H), 2.38-2.20 (m, 3H), 2.14-1.94 (m, 5H), 1.86 (dd, J=14.4, 10.0 Hz, 3H), 1.81-1.70 (m, 1H), 1.68-1.43 (m, 4H).

### Example 49. 4-Fluorophenyl ((1R,3R)-3-(6-((3-((cyclobutoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate

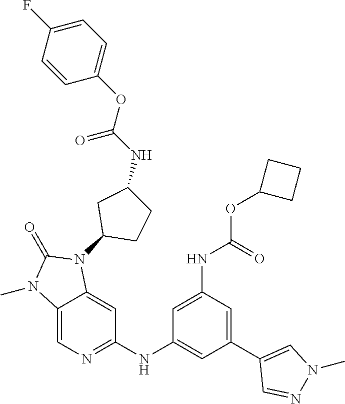

The title compound was prepared according to the procedures described in Example 43, with 4-fluorophenyl carbonochloridate replacing 2-methoxyethyl carbonochloridate (Step 4). LCMS calculated for C₃₄H₃₆FN₈O₅ (M+H)⁺: m/z=655.3; found: 655.3. ¹H NMR (400 MHz, DMSO-d₆) δ 9.73 (s, 1H), 9.61 (s, 1H), 8.00 (d, J=8.9 Hz, 3H), 7.71 (s, 1H), 7.38 (d, J=2.0 Hz, 1H), 7.34 (s, 1H), 7.26 (s, 1H), 7.24-7.10 (m, 4H), 6.89 (s, 1H), 4.96 (m, 2H), 4.26-4.19 (m, 1H), 3.87 (s, 3H), 3.34 (s, 3H), 2.38-2.27 (m, 3H), 2.17 (m, 2H), 2.10-1.94 (m, 4H), 1.83-1.53 (m, 3H).

### Example 50. Methyl ((1R,3R)-3-(6-((6-cyclopropylpyridin-2-yl)amino)-3-(methyl-d₃)-2-oxo-2,3-dihydro-1H-imidazo[4,5-c]pyridin-1-yl)cyclopentyl)carbamate

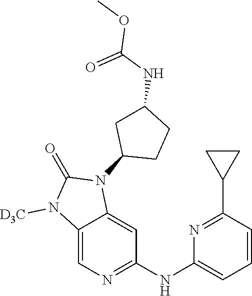 [SMILES: [2H]C([2H])([2H])n1c(=O)n([C@@H]2CC[C@@H](NC(=O)OC)C2)c2cc(Nc3cccc(C4CC4)n3)ncc21]

The title compound was prepared according to the procedures described in Example 53, with methyl ((1R,3R)-3-(6-chloro-3-(methyl-d₃)-2-oxo-2,3-dihydro-1H-imidazo[4,5-c]pyridin-1-yl)cyclopentyl)carbamate (Example 75, Step 1) replacing methyl ((1R,3R)-3-(6-chloro-3-methyl-2-oxo-2,3-dihydro-1H-imidazo[4,5-c]pyridin-1-yl)cyclopentyl)carbamate and 6-cyclopropylpyridin-2-amine replacing methyl (3-amino-5-(1-methyl-1H-pyrazol-4-yl)phenyl)carbamate in Step 4. LCMS calculated for C₂₂H₂₄D₃N₆O₃ (M+H)⁺: m/z=426.2; found: 426.2. ¹H NMR (500 MHz, DMSO) δ 11.23 (bs, 1H), 8.33 (s, 1H), 7.79 (t, J=7.9 Hz, 1H), 7.38 (d, J=7.1 Hz, 1H), 7.18 (s, 1H), 7.03-6.94 (m, 2H), 4.94 (p, J=8.7 Hz, 1H), 4.28-4.16 (m, 1H), 3.55 (s, 3H), 2.35-2.17 (m, 3H), 2.16-2.07 (m, 1H), 2.07-1.99 (m, 1H), 1.97-1.89 (m, 1H), 1.68-1.58 (m, 1H), 1.18-1.10 (m, 2H), 1.07-1.01 (m, 2H).

### Example 51. Methyl ((1R,3R)-3-(6-((6-(3-cyanophenyl)-4-(1-methyl-1H-pyrazol-4-yl)pyridin-2-yl)amino)-3-methyl-2-oxo-2,3-dihydro-1H-imidazo[4,5-c]pyridin-1-yl)cyclopentyl)carbamate

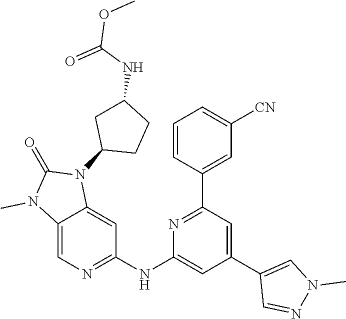

### Step 1. 3-(6-Amino-4-chloropyridin-2-yl)benzonitrile

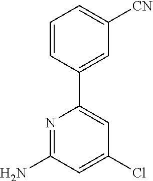 [SMILES: N#Cc1cccc(-c2cc(Cl)cc(N)n2)c1]

A reaction vial containing 4,6-dichloropyridin-2-amine (1.44 g, 8.85 mmol), (3-cyanophenyl)boronic acid (1 g, 6.81 mmol), sodium carbonate (2.16 g, 20.42 mmol) and [1,1′-bis(diphenylphosphino)ferrocene]dichloropalladium(II) (556 mg, 0.68 mmol) was evacuated and backfilled with nitrogen. 1,4-Dioxane (15 mL) and water (3 mL) were added to the reaction mixture, which was then stirred at 80° C. for 4 h. The reaction mixture was cooled to r.t and was diluted with EtOAc. The organic layer was subsequently washed with water and brine, dried over sodium sulfate, filtered, and the solvents were evaporated in vacuo. The crude material was purified by Biotage Isolera to give the product. LCMS calculated for C₁₂H₉ClN₃ (M+H)⁺: m/z=230.0; found 230.1.

### Step 2. 3-(6-Amino-4-(1-methyl-1H-pyrazol-4-yl)pyridin-2-yl)benzonitrile

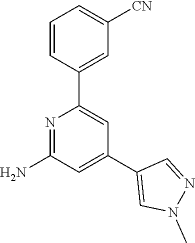 [SMILES: Cn1cc(-c2cc(N)nc(-c3cccc(C#N)c3)c2)cn1]

A reaction vial containing 3-(6-amino-4-chloropyridin-2-yl)benzonitrile (30 mg, 0.131 mmol), 1-methyl-4-(4,4,5,5-tetramethyl-1,3,2-dioxaborolan-2-yl)-1H-pyrazole (27 mg, 0.131 mmol), cesium carbonate (85 mg, 0.261 mmol) and chloro(2-dicyclohexylphosphino-2′,4′,6′-triisopropyl-1, l′-biphenyl)[2-(2′-amino-1, l′-biphenyl)]palladium(II) (10 mg, 0.013 mmol) was evacuated and backfilled with nitrogen. 1,4-Dioxane (1 mL) and water (0.2 mL) were added to the reaction mixture, which was then stirred at 80° C. for 30 min. The reaction mixture was cooled to r.t and was diluted with EtOAc. The organic layer was subsequently washed with water and brine, dried over sodium sulfate, filtered, and the solvents were evaporated in vacuo. The crude material was purified by Biotage Isolera to give the product. LCMS calculated for C₁₆H₁₄N₅ (M+H)⁺: m/z=276.1; found 276.1.

### Step 3. Methyl (1R,3R)-3-(6-((6-(3-cyanophenyl)-4-(1-methyl-1H-pyrazol-4-yl)pyridin-2-yl)amino)-3-methyl-2-oxo-2,3-dihydro-1H-imidazo[4,5-c]pyridin-1-yl)cyclopentyl)carbamate

The title compound was prepared according to the procedures described in Example 53, with 3-(6-amino-4-(1-methyl-1H-pyrazol-4-yl)pyridin-2-yl)benzonitrile replacing methyl (3-amino-5-(1-methyl-1H-pyrazol-4-yl)phenyl)carbamate (Step 4). LCMS calculated for C₃₀H₃₀N₉O₃ (M+H)⁺: m/z=564.2; found: 564.2. ¹H NMR (400 MHZ, DMSO-d₆) δ 10.50 (s, 1H), 8.60 (d, J=1.8 Hz, 1H), 8.48-8.42 (m, 2H), 8.24 (s, 1H), 8.13 (s, 1H), 7.96 (d, J=7.7 Hz, 1H), 7.92 (s, 1H), 7.76 (t, J=7.8 Hz, 1H), 7.47 (s, 1H), 7.39 (s, 1H), 7.33 (d, J=7.0 Hz, 1H), 4.93 (q, J=8.6 Hz, 1H), 4.13 (s, 1H), 3.95 (s, 3H), 3.54 (s, 3H), 3.37 (s, 3H), 2.28 (m, 1H), 2.16-1.86 (m, 4H), 1.58 (s, 1H).

### Example 52. Methyl ((1R,3R)-3-(3-(methyl-d₃)-6-((4-(morpholine-4-carbonyl)-8-(trifluoromethyl)quinolin-2-yl)amino)-2-oxo-2,3-dihydro-1H-imidazo[4,5-c]pyridin-1-yl)cyclopentyl)carbamate

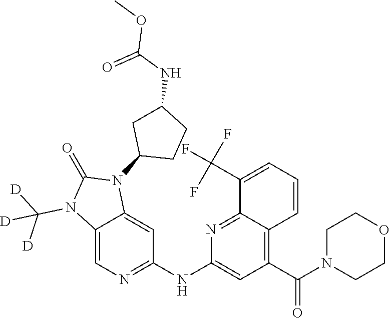 [SMILES: [2H]C([2H])([2H])n1c(=O)n([C@@H]2CC[C@@H](NC(=O)OC)C2)c2cc(Nc3cc(C(=O)N4CCOCC4)c4cccc(C(F)(F)F)c4n3)ncc21]

### Step 1. 2-Oxo-8-(trifluoromethyl)-1,2-dihydroquinoline-4-carboxylic acid

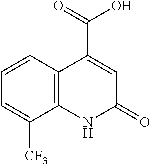 [SMILES: O=C(O)c1cc(=O)[nH]c2c(C(F)(F)F)cccc12]

To a vial containing a stir bar was added 7-(trifluoromethyl)indoline-2,3-dione (1.0 g, 4.65 mmol) and a solution of malonic acid (1.5 g, 14.0 mmol) in acetic acid (15.5 mL). The vial was sealed and the reaction mixture was heated to 110° C. and allowed to stir for 40 h. The reaction mixture was allowed to cool to r.t. and the solution was concentrated in vacuo. Water (50 mL) was added to the crude mixture and the resulting solid was filtered and used in the next reaction without further purification. LCMS calculated for C₁₁H₇F₃NO₃ (M+H)⁺: m/z=258.0; found: 258.0.

### Step 2. 2-Chloro-8-(trifluoromethyl)quinoline-4-carboxylic acid

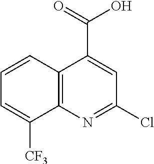 [SMILES: O=C(O)c1cc(Cl)nc2c(C(F)(F)F)cccc12]

To a vial containing 2-oxo-8-(trifluoromethyl)-1,2-dihydroquinoline-4-carboxylic acid (171 mg, 0.66 mmol) was added phosphorus oxychloride (1.24 mL, 13.3 mmol) and the reaction mixture was heated to 40° C. and allowed to stir for 4 h. Upon cooling to r.t. the solvent was evaporated in vacuo. The crude material was purified by Biotage Isolera to give the product as a brown solid. LCMS calculated for C₁₁H₆ClF₃NO₂ (M+H)⁺: m/z=276.0; found 276.0.

### Step 3. (2-Chloro-8-(trifluoromethyl)quinolin-4-yl)(morpholino)methanone

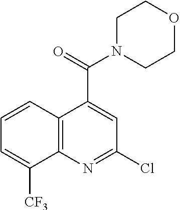 [SMILES: O=C(c1cc(Cl)nc2c(C(F)(F)F)cccc12)N1CCOCC1]

To a reaction vial containing 2-chloro-8-(trifluoromethyl)quinoline-4-carboxylic acid (100 mg, 0.36 mmol), morpholine (63 mg, 0.73 mmol), BOP (241 mg, 0.54 mmol) and DIPEA (95 μl, 0.54 mmol) was added DMF (4.0 mL). The reaction mixture was stirred at r.t for 1 h and subsequently diluted with water and extracted with ethyl acetate. The solvent was evaporated in vacuo. The crude material was purified by Biotage Isolera to give the product as a yellow solid. LCMS calculated for C₁₅H₁₃ClF₃N₂O₂ (M+H)⁺: m/z=345.1; found 345.1.

### Step 4. Methyl ((1R,3R)-3-(6-amino-3-(methyl-d₃)-2-oxo-2,3-dihydro-1H-imidazo[4,5-c]pyridin-1-yl)cyclopentyl)carbamate

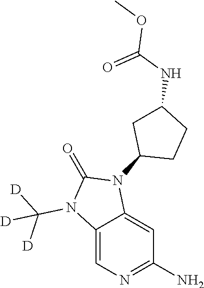 [SMILES: [2H]C([2H])([2H])n1c(=O)n([C@@H]2CC[C@@H](NC(=O)OC)C2)c2cc(N)ncc21]

A vial containing methyl ((1R,3R)-3-(6-chloro-3-(methyl-d3)-2-oxo-2,3-dihydro-1H-imidazo[4,5-c]pyridin-1-yl)cyclopentyl)carbamate (31 mg, 0.1 mmol, Example 75, Step 1), tert-butyl carbamate (33 mg, 0.28 mmol), tBuXPhos Pd G3 (8 mg, 9.5 μmol), and cesium carbonate (92 mg, 0.28 mmol) was evacuated and backfilled with nitrogen. 1,4-Dioxane (1 mL) was added to the reaction mixture, which was then stirred at 100° C. for 18 h. The reaction mixture was cooled to r.t., diluted with DCM, and filtered through celite. The solvents were evaporated in vacuo and the crude material was treated with a solution of 4:1 DCM/TFA (5.0 mL), which was stirred at r.t. for 2 h. The solvents were evaporated in vacuo and the crude material was purified by Biotage Isolera to give the desired product as a white solid. LCMS calculated for C₁₄H₁₇D₃N₅O₃ (M+H)⁺: m/z=309.2; found 309.2.

### Step 5. Methyl ((1R,3R)-3-(3-(methyl-d₃)-6-((4-(morpholine-4-carbonyl)-8-(trifluoromethyl)quinolin-2-yl)amino)-2-oxo-2,3-dihydro-1H-imidazo[4,5-c]pyridin-1-yl)cyclopentyl)carbamate

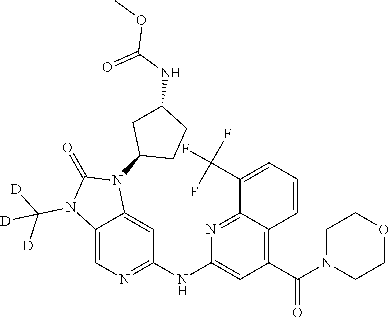 [SMILES: [2H]C([2H])([2H])n1c(=O)n([C@@H]2CC[C@@H](NC(=O)OC)C2)c2cc(Nc3cc(C(=O)N4CCOCC4)c4cccc(C(F)(F)F)c4n3)ncc21]

To a reaction vial was added methyl ((1R,3R)-3-(6-amino-3-(methyl-d3)-2-oxo-2,3-dihydro-1H-imidazo[4,5-c]pyridin-1-yl)cyclopentyl)carbamate (30 mg, 0.1 mmol), (2-chloro-8-(trifluoromethyl)quinolin-4-yl)(morpholino)methanone (34 mg, 0.1 mmol), XantPhos Pd G3 (9 mg, 9.7 μmol), and cesium carbonate (95 mg, 0.3 mmol). This was evacuated and backfilled with nitrogen 3 times. Subsequently 1,4-dioxane (1.0 mL) was added. The vial was sealed and heated to 100° C. for 16 h. Upon completion, the reaction was diluted with DCM, filtered through Celite and concentrated in vacuo. The crude material was diluted with MeCN and purified by prep-LCMS (XBridge C18 column, eluting with a gradient of acetonitrile/water containing 0.1% TFA, at flow rate of 60 mL/min. LCMS calculated for C₂₉H₂₈D₃F₃N₇O₅ (M+H)⁺: m/z=617.3; found: 617.3. ¹H NMR (500 MHZ, DMSO-d₆) δ 10.98 (s, 1H), 8.28 (s, 1H), 8.16 (s, 1H), 8.15 (s, 1H), 7.98 (d, J=8.9 Hz, 1H), 7.59 (dd, J=8.4, 7.9 Hz, 1H), 7.38 (s, 1H), 7.33 (d, J=7.0 Hz, 1H), 4.92-4.82 (m, 1H), 4.30-4.22 (m, 1H), 3.90-3.70 (m, 4H), 3.55 (s, 3H), 3.53-3.41 (m, 2H), 3.32-3.14 (m, 2H), 2.44-2.36 (m, 1H), 2.27-2.17 (m, 1H), 2.15-2.08 (m, 2H), 2.05-1.89 (m, 1H), 1.67-1.56 (m, 1H).

### Example 53. Methyl ((1R,3R)-3-(6-((3-((methoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate

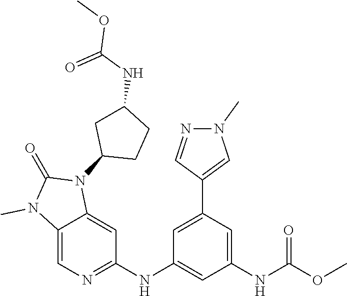

### Step 1. Methyl (3-bromo-5-nitrophenyl)carbamate

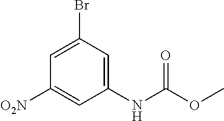

To a flask containing 3-bromo-5-nitroaniline (1.50 g, 6.91 mmol) and DIPEA (0.98 g, 1.328 mL, 7.60 mmol) in acetonitrile (3.5 ml) was added methyl carbonochloridate (0.72 g, 0.589 mL, 7.60 mmol). The reaction mixture was stirred at r.t. for 3 h, then diluted with water (30 mL) and filtered. The solid was washed with water (30 mL) for 3 times and dried in vacuo. The obtained crude product was used in the next step without further purification.

### Step 2. Methyl (3-amino-5-bromophenyl)carbamate

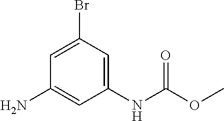

The title compound was prepared according to the procedures described in Intermediate A, with methyl (3-bromo-5-nitrophenyl)carbamate replacing tert-butyl ((1R,3R)-3-((2-chloro-5-nitropyridin-4-yl)amino)cyclopentyl)carbamate in Step 2. LCMS calculated for C₈H₁₀BrN₂O₂ (M+H)⁺: m/z=245.0/247.0; found 245.1/247.0.

### Step 3. Methyl (3-amino-5-(1-methyl-1H-pyrazol-4-yl)phenyl)carbamate

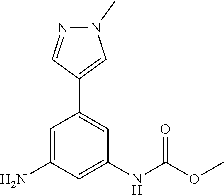 [SMILES: COC(=O)Nc1cc(N)cc(-c2cnn(C)c2)c1]

A vial containing methyl (3-amino-5-bromophenyl)carbamate (25 mg, 0.102 mmol), 1-methyl-4-(4,4,5,5-tetramethyl-1,3,2-dioxaborolan-2-yl)-1H-pyrazole (26 mg, 0.122 mmol), potassium carbonate (28 mg, 0.204 mmol) and (1, l′-bis(diphenylphosphino)ferrocene)-dichloropalladium(II) dichloromethane adduct (8 mg, 10.2 μmol) was evacuated and backfilled with nitrogen three times, followed by the addition of 1,4-dioxane (0.5 mL) and water (50 μL). The vial was sealed and heated to 90° C. for 4 h. After cooling to room temperature, the mixture was filtered through a SiliaPrep SPE thiol cartridge (SPE-R51030B-06P) and washed with acetonitrile. The mixture was then diluted with acetonitrile and purified with prep-LCMS (XBridge C18 column, eluting with a gradient of acetonitrile/water containing 0.1% TFA, at flow rate of 60 mL/min). LCMS calculated for C₁₂H₁₅N₄O₂ (M+H)⁺: m/z=247.1; found 247.1.

### Step 4. Methyl ((1R,3R)-3-(6-((3-((methoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate

A vial containing methyl ((1R,3R)-3-(6-chloro-3-methyl-2-oxo-2,3-dihydro-1H-imidazo[4,5-c]pyridin-1-yl)cyclopentyl)carbamate (15 mg. 46 μmol, Intermediate A), methyl (3-amino-5-(1-methyl-1H-pyrazol-4-yl)phenyl)carbamate (23 mg, 92 μmol), cesium carbonate (60 mg, 0.185 mmol), 4,5-bis(diphenylphosphino)-9,9-dimethylxanthene (8 mg, 14 μmol) and tris(dibenzylideneacetone)dipalladium(0) (6 mg, 6.9 μmol) was evacuated and backfilled with nitrogen three times, followed by the addition of dioxane (0.6 ml). The vial was sealed and heated to 100° C. for 16 h. After cooling to r.t., the mixture was filtered through a SiliaPrep SPE thiol cartridge (SPE-R51030B-06P) and washed with acetonitrile. The mixture was then diluted with acetonitrile and purified with prep-LCMS (XBridge C18 column, eluting with a gradient of acetonitrile/water containing 0.1% TFA, at flow rate of 60 mL/min). LCMS calculated for C₂₆H₃₁N₈O₅ (M+H)⁺: m/z=535.2; found: 535.2. ¹H NMR (600 MHz, DMSO) δ 9.75 (s, 1H), 9.62 (bs, 1H), 8.01 (s, 1H), 7.98 (s, 1H), 7.71 (s, 1H), 7.41 (s, 1H), 7.36-7.30 (m, 2H), 7.27 (s, 1H), 6.86 (s, 1H), 4.91 (p, J=8.6 Hz, 1H), 4.19-4.13 (m, 1H), 3.88 (s, 3H), 3.68 (s, 3H), 3.53 (s, 3H), 3.34 (s, 3H), 2.25 (dt, J=13.5, 7.8 Hz, 1H), 2.14-2.05 (m, 2H), 2.03-1.95 (m, 1H), 1.89 (ddd, J=14.1, 9.5, 5.0 Hz, 1H), 1.62-1.54 (m, 1H).

### Example 54. Methyl ((1R,3R)-3-(6-((3-cyclopropylimidazo[1,2-b]pyridazin-6-yl)amino)-3-(methyl-d3)-2-oxo-2,3-dihydro-1H-imidazo[4,5-c]pyridin-1-yl)cyclopentyl)carbamate

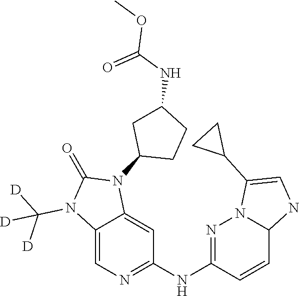 [SMILES: [2H]C([2H])([2H])n1c(=O)n([C@@H]2CC[C@@H](NC(=O)OC)C2)c2cc(NC3=NN4C(C5CC5)=CNC4C=C3)ncc21]

### Step 1. 3-Cyclopropylimidazo[1,2-b]pyridazin-6-amine

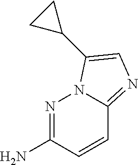 [SMILES: Nc1ccc2ncc(C3CC3)n2n1]

A reaction vial containing 3-bromoimidazo[1,2-b]pyridazin-6-amine (50 mg, 0.24 mmol), cyclopropylboronic acid (30 mg, 0.35 mmol), potassium phosphate (100 mg, 0.47 mmol) and XPhos Pd G3 (20 mg, 0.02 mmol) was evacuated and backfilled with nitrogen. Toluene (2.0 mL) and water (0.2 mL) were added to the reaction mixture, which was then stirred at 100° C. for 18 h. The reaction mixture was cooled to r.t and was diluted with EtOAc. The organic layer was subsequently washed with water and brine, dried over sodium sulfate, filtered, and the solvents were evaporated in vacuo. The crude material was diluted with MeCN and purified by prep-LCMS (XBridge C18 column, eluting with a gradient of acetonitrile/water containing 0.1% TFA, at flow rate of 60 mL/min. LCMS calculated for C₉H₁₁N₄ (M+H)⁺: m/z=175.1; found 175.1.

### Step 2. Methyl ((1R,3R)-3-(6-((3-cyclopropylimidazo[1,2-b]pyridazin-6-yl)amino)-3-(methyl-d3)-2-oxo-2,3-dihydro-1H-imidazo[4,5-c]pyridin-1-yl)cyclopentyl)carbamate

The title compound was prepared according to the procedures described in Example 32, Step 2 with 3-cyclopropylimidazo[1,2-b]pyridazin-6-amine replacing 6-(1-methyl-1H-pyrazol-4-yl)quinolin-2-amine and with methyl ((1R,3R)-3-(6-chloro-3-(methyl-d3)-2-oxo-2,3-dihydro-1H-imidazo[4,5-c]pyridin-1-yl)cyclopentyl)carbamate (Example 75, Step 1) replacing methyl ((1R,3R)-3-(6-chloro-3-methyl-2-oxo-2,3-dihydro-1H-imidazo[4,5-c]pyridin-1-yl)cyclopentyl)carbamate. LCMS calculated for C₂₃H₂₄D₃N₈O₃ (M+H)⁺: m/z=466.2; found: 466.2.

### Example 55. Methyl ((1R,3R)-3-(6-((3-((cyclopropoxycarbonyl)amino)-5-methoxyphenyl)amino)-3-methyl-2-oxo-2,3-dihydro-1H-imidazo[4,5-c]pyridin-1-yl)cyclopentyl)carbamate

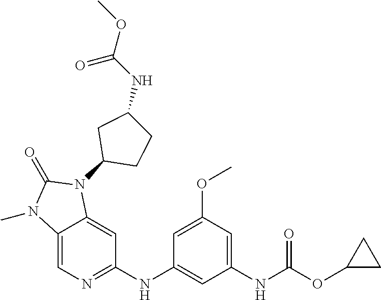 [SMILES: COC(=O)N[C@@H]1CC[C@@H](n2c(=O)n(C)c3cnc(Nc4cc(NC(=O)OC5CC5)cc(OC)c4)cc32)C1]

### Step 1. Cyclopropyl (3-methoxy-5-nitrophenyl)carbamate

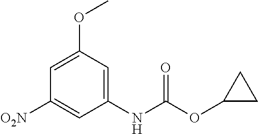 [SMILES: COc1cc(NC(=O)OC2CC2)cc([N+](=O)[O-])c1]

To a flask containing triphosgene (176 mg, 0.595 mmol) in CH₂Cl₂ (4.0 mL) at 0° C. was added 1 drop of pyridine. Then a solution of cyclopropanol (104 mg, 1.784 mmol) and pyridine (144 μl, 1.784 mmol) in CH₂Cl₂ (4.0 mL) was added to the flask via dropping addition funnel at the rate around 2 drop/second. The reaction mixture was stirred at 0° C. for another 30 min. The reaction mixture was concentrated in vacuo, diluted with hexanes and filtered. The filtrate was concentrated to get chloroformate. To another vial containing 3-methoxy-5-nitroaniline (200 mg, 1.19 mmol) and DIPEA (208 μl, 1.19 mmol) in THF (4 mL) was added the crude chloroformate from above. The reaction was stirred at 40° C. for 2h. The reaction mixture was diluted with water (20 mL) and filtered. The solid was washed with water (5 mL) for 3 times and dried in vacuo. The obtained crude product was used in the next step without further purification.

### Step 2. Cyclopropyl (3-amino-5-methoxyphenyl)carbamate

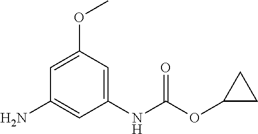 [SMILES: COc1cc(N)cc(NC(=O)OC2CC2)c1]

The title compound was prepared according to the procedures described in Intermediate A, with cyclopropyl (3-methoxy-5-nitrophenyl)carbamate replacing tert-butyl ((1R,3R)-3-((2-chloro-5-nitropyridin-4-yl)amino)cyclopentyl)carbamate in Step 2. LCMS calculated for C₁₁H₁₅N₂O₃ (M+H)⁺: m/z=223.1; found 223.1.

### Step 3. Methyl ((1R,3R)-3-(6-((3-((cyclopropoxycarbonyl)amino)-5-methoxyphenyl)amino)-3-methyl-2-oxo-2,3-dihydro-1H-imidazo[4,5-c]pyridin-1-yl)cyclopentyl)carbamate

The title compound was prepared according to the procedures described in Example 53, with cyclopropyl (3-amino-5-methoxyphenyl)carbamate replacing methyl (3-amino-5-(1-methyl-1H-pyrazol-4-yl)phenyl)carbamate in Step 4. LCMS calculated for C₂₅H₃₁N₆O₆ (M+H)⁺: m/z=511.2; found: 511.2. ¹H NMR (600 MHz, DMSO) δ 9.67 (s, 1H), 9.42 (bs, 1H), 7.97 (s, 1H), 7.35 (d, J=7.0 Hz, 1H), 7.15 (s, 1H), 6.87-6.79 (m, 2H), 6.76 (s, 1H), 4.90 (p, J=8.7 Hz, 1H), 4.20-4.14 (m, 1H), 4.09-4.03 (m, 1H), 3.71 (s, 3H), 3.54 (s, 3H), 3.32 (s, 3H), 2.24 (dt, J=13.5, 7.9 Hz, 1H), 2.19-2.03 (m, 2H), 2.03-1.95 (m 1H), 1.88 (ddd, J=14.0, 9.5, 5.0 Hz, 1H), 1.63-1.57 (m, 1H), 0.74-0.65 (m, 4H).

### Example 56. Methyl ((1R,3R)-3-(6-((3-((cyclobutoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate

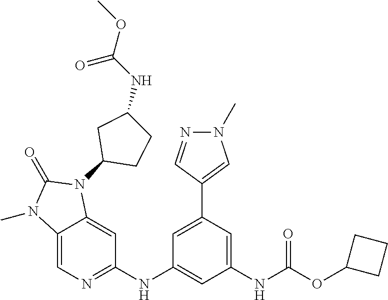 [SMILES: COC(=O)N[C@@H]1CC[C@@H](n2c(=O)n(C)c3cnc(Nc4cc(NC(=O)OC5CCC5)cc(-c5cnn(C)c5)c4)cc32)C1]

### Step 1. 3-(1-Methyl-1H-pyrazol-4-yl)-5-nitroaniline

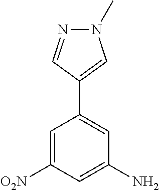 [SMILES: Cn1cc(-c2cc(N)cc([N+](=O)[O-])c2)cn1]

A vial containing 3-bromo-5-nitroaniline (1.00 g, 4.61 mmol), 1-methyl-4-(4,4,5,5-tetramethyl-1,3,2-dioxaborolan-2-yl)-1H-pyrazole (1.06 g, 5.07 mmol), potassium phosphate (2 g, 9.22 mmol) and (1,1′-bis(diphenylphosphino)ferrocene)-dichloropalladium(II) dichloromethane adduct (151 mg, 0.184 mmol) was evacuated and backfilled with nitrogen three times, followed by the addition of 1,4-dioxane (10 mL) and water (1.0 mL). The vial was sealed and heated to 90° C. for 4 h. The reaction mixture was cooled to r.t., then diluted with water (50 mL) and filtered. The solid was washed with water (30 mL) for 3 times and dried in vacuo. The obtained crude product was used in the next step without further purification. LCMS calculated for C₁₀H₁₁N₄O₂ (M+H)⁺: m/z=219.1; found: 219.1.

### Step 2. Cyclobutyl (3-(1-methyl-1H-pyrazol-4-yl)-5-nitrophenyl)carbamate

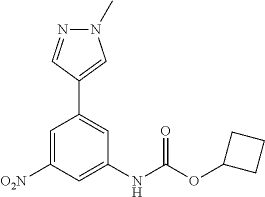 [SMILES: Cn1cc(-c2cc(NC(=O)OC3CCC3)cc([N+](=O)[O-])c2)cn1]

The title compound was prepared according to the procedures described in Example 55, with 3-(1-methyl-1H-pyrazol-4-yl)-5-nitroaniline replacing 3-methoxy-5-nitroaniline in Step 1. LCMS calculated for C₁₅H₁₇N₄O₄ (M+H)⁺: m/z=317.1; found: 317.1.

### Step 3. Cyclobutyl (3-amino-5-(1-methyl-1H-pyrazol-4-yl)phenyl) carbamate

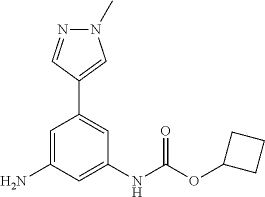 [SMILES: Cn1cc(-c2cc(N)cc(NC(=O)OC3CCC3)c2)cn1]

The title compound was prepared according to the procedures described in Intermediate A, with cyclobutyl (3-(1-methyl-1H-pyrazol-4-yl)-5-nitrophenyl)carbamate replacing tert-butyl ((1R,3R)-3-((2-chloro-5-nitropyridin-4-yl)amino)cyclopentyl)carbamate in Step 2. The obtained crude product was purified by Biotage Isolera to give the desired product as colorless oil. LCMS calculated for C₁₅H₁₉N₄O₂ (M+H)⁺: m/z=287.1; found: 287.1.

### Step 4. Methyl ((1R,3R)-3-(6-((3-((cyclobutoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate

The title compound was prepared according to the procedures described in Example 53, with cyclobutyl (3-amino-5-(1-methyl-1H-pyrazol-4-yl)phenyl)carbamate replacing methyl (3-amino-5-(1-methyl-1H-pyrazol-4-yl)phenyl)carbamate in Step 4. LCMS calculated for C₂₉H₃₅N₈O₅ (M+H)⁺: m/z=575.3; found: 575.3. ¹H NMR (600 MHz, DMSO) δ 9.71 (s, 1H), 9.53 (bs, 1H), 8.00 (s, 1H), 7.98 (s, 1H), 7.70 (s, 1H), 7.40 (s, 1H), 7.36-7.28 (m, 3H), 6.84 (s, 1H), 4.98-4.86 (m, 2H), 4.19-4.13 (m, 1H), 3.88 (s, 3H), 3.53 (s, 3H), 3.33 (s, 3H), 2.36-2.28 (m, 2H), 2.25 (dt, J=13.6, 7.9 Hz, 1H), 2.16-1.94 (m, 4H), 1.89 (ddd, J=13.9, 9.5, 5.0 Hz, 1H), 1.81-1.72 (m, 1H), 1.66-1.56 (m, 2H).

### Example 57. Methyl ((1R,3R)-3-(6-((3-(cyclopropanecarboxamido)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate

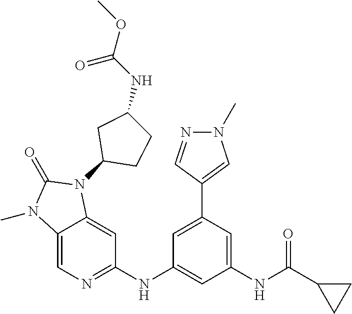 [SMILES: COC(=O)N[C@@H]1CC[C@@H](n2c(=O)n(C)c3cnc(Nc4cc(NC(=O)C5CC5)cc(-c5cnn(C)c5)c4)cc32)C1]

### Step 1. N-(3-(1-Methyl-1H-pyrazol-4-yl)-5-nitrophenyl)cyclopropanecarboxamide

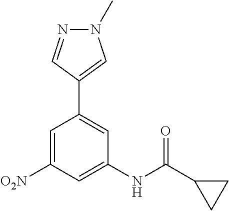 [SMILES: Cn1cc(-c2cc(NC(=O)C3CC3)cc([N+](=O)[O-])c2)cn1]

To a vial containing 3-(1-methyl-1H-pyrazol-4-yl)-5-nitroaniline (50 mg, 0.229 mmol), DIPEA (88 μl, 0.504 mmol) in CH₂Cl₂ (1.0 mL) at 0° C. was added cyclopropanecarbonyl chloride (36 mg, 0.344 mmol) After stirring at r.t. for 4 h, the reaction mixture was quenched with saturated aqueous NaHCO₃ and the mixture was extracted with CH₂Cl₂. The organic phase was washed with brine, dried over MgSO₄, filtered and the solvent was evaporated in vacuo. The obtained crude product was used in the next step without further purification. LCMS calculated for C₁₄H₁₅N₄O₃ (M+H)⁺: m/z=287.1; found: 287.1.

### Step 2. N-(3-Amino-5-(1-methyl-1H-pyrazol-4-yl)phenyl)cyclopropanecarboxamide

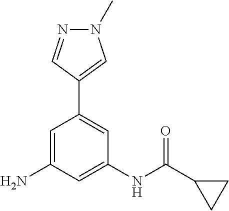 [SMILES: Cn1cc(-c2cc(N)cc(NC(=O)C3CC3)c2)cn1]

The title compound was prepared according to the procedures described in Intermediate A, with N-(3-(1-methyl-1H-pyrazol-4-yl)-5-nitrophenyl)cyclopropanecarboxamide replacing tert-butyl ((1R,3R)-3-((2-chloro-5-nitropyridin-4-yl)amino)cyclopentyl)carbamate in Step 2. The obtained crude product was purified by Biotage Isolera to give the desired product as colorless oil. LCMS calculated for C₁₄H₁₇N₄O (M+H)⁺: m/z=257.1; found: 257.1.

### Step 3. Methyl ((1R,3R)-3-(6-((3-(cyclopropanecarboxamido)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate

The title compound was prepared according to the procedures described in Example 53, with N-(3-amino-5-(1-methyl-1H-pyrazol-4-yl)phenyl)cyclopropanecarboxamide replacing methyl (3-amino-5-(1-methyl-1H-pyrazol-4-yl)phenyl)carbamate in Step 4. LCMS calculated for C₂₈H₃₃N₈O₄ (M+H)⁺: m/z=545.3; found: 545.3. ¹H NMR (600 MHZ, DMSO) δ 10.30 (s, 1H), 9.67 (bs, 1H), 8.03 (s, 1H), 7.99 (s, 1H), 7.73 (s, 1H), 7.62 (s, 1H), 7.43 (s, 1H), 7.33 (d, J=7.0 Hz, 1H), 7.26 (s, 1H), 6.88 (s, 1H), 4.91 (p, J=8.5 Hz, 1H), 4.19-4.12 (m, 1H), 3.88 (s, 3H), 3.53 (s, 3H), 3.34 (s, 3H), 2.25 (dt, J=13.5, 7.8 Hz, 1H), 2.15-2.05 (m, 2H), 2.04-1.95 (m, 1H), 1.89 (ddd, J=13.9, 9.4, 4.9 Hz, 1H), 1.84-1.79 (m, 1H), 1.62-1.54 (m, 1H), 0.86-0.77 (m, 4H).

### Example 58. Methyl ((1R,3R)-3-(3-(methyl-d3)-6-((8-(1-methylcyclopropyl)quinolin-2-yl)amino)-2-oxo-2,3-dihydro-1H-imidazo[4,5-c]pyridin-1-yl)cyclopentyl)carbamate

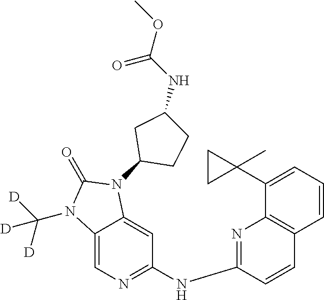 [SMILES: [2H]C([2H])([2H])n1c(=O)n([C@@H]2CC[C@@H](NC(=O)OC)C2)c2cc(Nc3ccc4cccc(C5(C)CC5)c4n3)ncc21]

### Step 1. 8-(1-Methylcyclopropyl)quinolin-2-amine

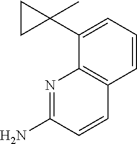

A reaction vial containing 8-bromoquinolin-2-amine (50 mg, 0.22 mmol), potassium cyclopropylmethyltrifluoroborate (55 mg, 0.34 mmol), potassium phosphate (95 mg, 0.45 mmol) and RuPhos Pd G4 (19 mg, 0.02 mmol) was evacuated and backfilled with nitrogen. Dioxane (2.0 mL) and water (0.2 mL) were added to the reaction mixture, which was then stirred at 100° C. for 18 h. The reaction mixture was cooled to r.t and was diluted with EtOAc. The organic layer was subsequently washed with water and brine, dried over sodium sulfate, filtered, and the solvents were evaporated in vacuo. The crude material was diluted with MeCN and purified by prep-LCMS (XBridge C18 column, eluting with a gradient of acetonitrile/water containing 0.1% TFA, at flow rate of 60 mL/min. LCMS calculated for C₁₃H₁₅N₂ (M+H)⁺: m/z=199.1; found 199.1.

### Step 2. Methyl ((1R,3R)-3-(3-(methyl-d3)-6-((8-(1-methylcyclopropyl)quinolin-2-yl)amino)-2-oxo-2,3-dihydro-1H-imidazo[4,5-c]pyridin-1-yl)cyclopentyl)carbamate

The title compound was prepared according to the procedures described in Example 32, Step 2 with 8-(1-methylcyclopropyl)quinolin-2-amine replacing 6-(1-methyl-1H-pyrazol-4-yl)quinolin-2-amine and with methyl ((1R,3R)-3-(6-chloro-3-(methyl-d3)-2-oxo-2,3-dihydro-1H-imidazo[4,5-c]pyridin-1-yl)cyclopentyl)carbamate (Example 75, Step 1) replacing methyl ((1R,3R)-3-(6-chloro-3-methyl-2-oxo-2,3-dihydro-1H-imidazo[4,5-c]pyridin-1-yl)cyclopentyl)carbamate. LCMS calculated for C₂₇H₂₈D₃N₆O₃ (M+H)⁺: m/z=490.3; found: 490.3.

### Example 59. Methyl ((1R,3R)-3-(6-((3-(3-fluoro-1-(methyl-d3)-1H-pyrazol-4-yl)imidazo[1,2-b]pyridazin-6-yl)amino)-3-methyl-2-oxo-2,3-dihydro-1H-imidazo[4,5-c]pyridin-1-yl)cyclopentyl)carbamate

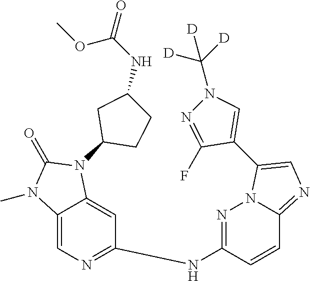 [SMILES: [2H]C([2H])([2H])n1cc(-c2cnc3ccc(Nc4cc5c(cn4)n(C)c(=O)n5[C@@H]4CC[C@@H](NC(=O)OC)C4)nn23)c(F)n1]

### Step 1. 3-(3-Fluoro-1-(methyl-d3)-1H-pyrazol-4-yl)imidazo[1,2-b]pyridazin-6-amine

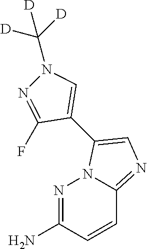 [SMILES: [2H]C([2H])([2H])n1cc(-c2cnc3ccc(N)nn23)c(F)n1]

The title compound was prepared according to the procedures described in Example 32, Step 1 with 3-bromoimidazo[1,2-b]pyridazin-6-amine replacing 6-bromoquinolin-2-amine and with (3-fluoro-1-(methyl-d3)-1H-pyrazol-4-yl)boronic acid replacing (1-methyl-1H-pyrazol-4-yl)boronic acid. LCMS calculated for C₁₀H₇D₃FN₆ (M+H)⁺: m/z=236.1; found: 236.1.

### Step 2. Methyl ((1R,3R)-3-(6-((3-(3-fluoro-1-(methyl-d3)-1H-pyrazol-4-yl)imidazo[1,2-b]pyridazin-6-yl)amino)-3-methyl-2-oxo-2,3-dihydro-1H-imidazo[4,5-c]pyridin-1-yl)cyclopentyl)carbamate

The title compound was prepared according to the procedures described in Example 32, Step 2 with 3-(3-fluoro-1-(methyl-d3)-1H-pyrazol-4-yl)imidazo[1,2-b]pyridazin-6-amine replacing 6-(1-methyl-1H-pyrazol-4-yl)quinolin-2-amine. LCMS calculated for C₂₄H₂₃D₃FN₁₀O₃ (M+H)⁺: m/z=524.2; found 524.2.

### Example 60. Cyclobutyl (3-((1-cyclopentyl-3-methyl-2-oxo-2,3-dihydro-1H-imidazo[4,5-c]pyridin-6-yl)amino)-5-(1-methyl-1H-pyrazol-4-yl)phenyl)carbamate

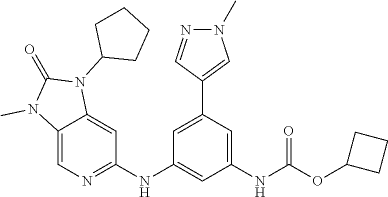 [SMILES: Cn1cc(-c2cc(NC(=O)OC3CCC3)cc(Nc3cc4c(cn3)n(C)c(=O)n4C3CCCC3)c2)cn1]

### Step 1. 6-Chloro-1-cyclopentyl-3-methyl-1,3-dihydro-2H-imidazo[4,5-c]pyridin-2-one

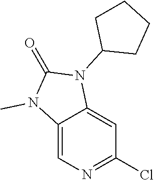 [SMILES: Cn1c(=O)n(C2CCCC2)c2cc(Cl)ncc21]

The title compound was prepared according to the procedures described in Intermediate A, with cyclopentanamine replacing tert-butyl ((1R,3R)-3-aminocyclopentyl)carbamate in Step 1. LCMS calculated for C₁₂H₁₅ClN₃O (M+H)⁺: m/z=252.1; found: 252.1.

### Step 2. Cyclobutyl (3-((1-cyclopentyl-3-methyl-2-oxo-2,3-dihydro-1H-imidazo[4,5-c]pyridin-6-yl)amino)-5-(1-methyl-1H-pyrazol-4-yl)phenyl)carbamate

A vial containing 6-chloro-1-cyclopentyl-3-methyl-1,3-dihydro-2H-imidazo[4,5-c]pyridin-2-one (15 mg, 60 μmol), cyclobutyl (3-amino-5-(1-methyl-1H-pyrazol-4-yl)phenyl)carbamate (34 mg, 0.119 mmol, Example 56, Step 3), cesium carbonate (39 mg, 0.119 mmol), 4,5-bis(diphenylphosphino)-9,9-dimethylxanthene (10 mg, 18.0 μmol) and tris(dibenzylideneacetone)dipalladium(0) (8 mg, 8.9 μmol) was evacuated and backfilled with nitrogen three times, followed by the addition of 1,4-dioxane (0.6 mL). The vial was sealed and heated to 100° C. for 16 h. After cooling to r.t., the mixture was filtered through a SiliaPrep SPE thiol cartridge (SPE-R51030B-06P) and washed with acetonitrile. The mixture was then diluted with acetonitrile and purified with prep-LCMS (XBridge C18 column, eluting with a gradient of acetonitrile/water containing 0.1% TFA, at flow rate of 60 mL/min). LCMS calculated for C₂₇H₃₂N₇O₃ (M+H)⁺: m/z=502.3; found: 502.3. ¹H NMR (400 MHZ, DMSO) δ 9.72 (s, 1H), 9.51 (bs, 1H), 8.00 (s, 1H), 7.97 (s, 1H), 7.70 (s, 1H), 7.39 (s, 1H), 7.30 (m, 2H), 6.83 (s, 1H), 4.94 (p, J=7.5 Hz, 1H), 4.76 (p, J=8.5 Hz, 1H), 3.88 (s, 3H), 3.34 (s, 3H), 2.31 (m, 2H), 2.07 (m, 2H), 1.96 (m, 4H), 1.86-1.71 (m, 3H), 1.70-1.61 (m, 3H).

### Example 61. Cyclobutyl (3-((1-cyclopentyl-3-methyl-2-oxo-2,3-dihydro-1H-imidazo[4,5-c]pyridin-6-yl)amino)-5-(1-cyclopropyl-1H-pyrazol-4-yl)phenyl)carbamate

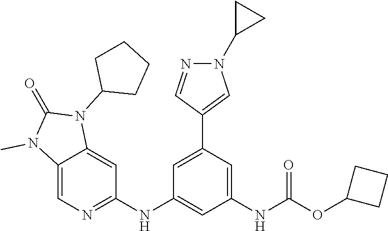 [SMILES: Cn1c(=O)n(C2CCCC2)c2cc(Nc3cc(NC(=O)OC4CCC4)cc(-c4cnn(C5CC5)c4)c3)ncc21]

### Step 1. Cyclobutyl (3-bromo-5-nitrophenyl)carbamate

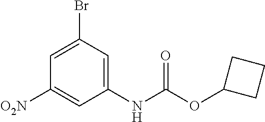 [SMILES: O=C(Nc1cc(Br)cc([N+](=O)[O-])c1)OC1CCC1]

The title compound was prepared according to the procedures described in Example 55, with cyclobutanol replacing cyclopropanol in Step 1.

### Step 2. Cyclobutyl (3-amino-5-bromophenyl)carbamate

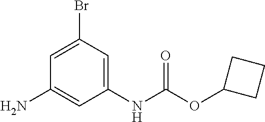 [SMILES: Nc1cc(Br)cc(NC(=O)OC2CCC2)c1]

The title compound was prepared according to the procedures described in Intermediate A, with cyclobutyl (3-bromo-5-nitrophenyl)carbamate replacing tert-butyl ((1R,3R)-3-((2-chloro-5-nitropyridin-4-yl)amino)cyclopentyl)carbamate in Step 2. LCMS calculated for C₁₁H₁₄BrN₂O₂ (M+H)⁺: m/z=285.0/287.0; found 285.0/287.1.

### Step 3. Cyclobutyl (3-amino-5-(1-cyclopropyl-1H-pyrazol-4-yl)phenyl)carbamate

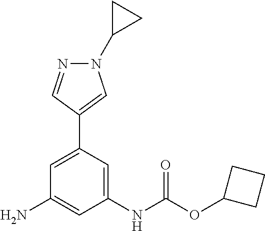 [SMILES: Nc1cc(NC(=O)OC2CCC2)cc(-c2cnn(C3CC3)c2)c1]

A vial containing cyclobutyl (3-amino-5-bromophenyl)carbamate (200 mg, 0.701 mmol), 1-cyclopropyl-4-(4,4,5,5-tetramethyl-1,3,2-dioxaborolan-2-yl)-1H-pyrazole (197 mg, 0.842 mmol), potassium carbonate (194 mg, 1.403 mmol) and (1,1′-bis(diphenylphosphino)ferrocene)-dichloropalladium(II) dichloromethane adduct (57 mg, 0.070 mmol) was evacuated and backfilled with nitrogen three times, followed by the addition of 1,4-dioxane (4.0 mL) and water (0.4 mL). The vial was sealed and heated to 90° C. for 4 h. After cooling to room temperature, the mixture was filtered through a SiliaPrep SPE thiol cartridge (SPE-R51030B-06P) and washed with CH₂Cl₂. The filtrate was concentrated in vacuo and was washed with aqueous saturated sodium bicarbonate solution. The organic phase was washed with brine, dried over MgSO₄ and concentrated in vacuo. The obtained crude product was purified by Biotage Isolera to give the desired product as white foam. LCMS calculated for C₁₇H₂₁N₄O₂ (M+H)⁺: m/z=313.2; found 313.2.

### Step 4. Cyclobutyl (3-((1-cyclopentyl-3-methyl-2-oxo-2,3-dihydro-1H-imidazo[4,5-c]pyridin-6-yl)amino)-5-(1-cyclopropyl-1H-pyrazol-4-yl)phenyl)carbamate

The title compound was prepared according to the procedures described in Example 60, with cyclobutyl (3-amino-5-(1-cyclopropyl-1H-pyrazol-4-yl)phenyl)carbamate replacing cyclobutyl (3-amino-5-(1-methyl-1H-pyrazol-4-yl)phenyl)carbamate in Step 5. LCMS calculated for C₂₉H₃₄N₇O₃ (M+H)⁺: m/z=528.3; found: 528.2. ¹H NMR (400 MHZ, DMSO) δ 9.69 (s, 1H), 9.46 (bs, 1H), 8.08 (s, 1H), 7.97 (s, 1H), 7.69 (s, 1H), 7.39 (s, 1H), 7.31 (m, 2H), 6.82 (s, 1H), 4.94 (p, J=7.5 Hz, 1H), 4.76 (p, J=8.6 Hz, 1H), 3.77 (m, 1H), 3.34 (s, 3H), 2.31 (m, 2H), 2.14-2.01 (m, 2H), 1.96 (m, 4H), 1.89-1.72 (m, 3H), 1.63 (m, 3H), 1.12-0.94 (m, 4H).

### Example 62. Cyclobutyl (3-(1-cyclopropyl-1H-pyrazol-4-yl)-5-((3-methyl-2-oxo-1-(tetrahydrofuran-3-yl)-2,3-dihydro-1H-imidazo[4,5-c]pyridin-6-yl)amino)phenyl)carbamate

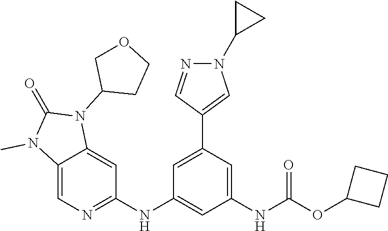 [SMILES: Cn1c(=O)n(C2CCOC2)c2cc(Nc3cc(NC(=O)OC4CCC4)cc(-c4cnn(C5CC5)c4)c3)ncc21]

### Step 1. 6-Chloro-3-methyl-1-(tetrahydrofuran-3-yl)-1,3-dihydro-2H-imidazo[4,5-c]pyridin-2-one

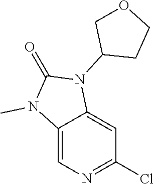 [SMILES: Cn1c(=O)n(C2CCOC2)c2cc(Cl)ncc21]

The title compound was prepared according to the procedures described in Intermediate A, with tetrahydrofuran-3-amine replacing tert-butyl ((1R,3R)-3-aminocyclopentyl)carbamate in Step 1. LCMS calculated for C₁₁H₁₃ClN₃O₂ (M+H)⁺: m/z=254.1; found: 254.1.

### Step 2. Cyclobutyl (3-(1-cyclopropyl-1H-pyrazol-4-yl)-5-((3-methyl-2-oxo-1-(tetrahydrofuran-3-yl)-2,3-dihydro-1H-imidazo[4,5-c]pyridin-6-yl)amino)phenyl)carbamate

The title compound was prepared according to the procedures described in Example 60, with 6-chloro-3-methyl-1-(tetrahydrofuran-3-yl)-1,3-dihydro-2H-imidazo[4,5-c]pyridin-2-one replacing 6-chloro-1-cyclopentyl-3-methyl-1,3-dihydro-2H-imidazo[4,5-c]pyridin-2-one and cyclobutyl (3-amino-5-(1-cyclopropyl-1H-pyrazol-4-yl)phenyl)carbamate replacing cyclobutyl (3-amino-5-(1-methyl-1H-pyrazol-4-yl)phenyl)carbamate in Step 5. LCMS calculated for C₂₈H₃₂N₇O₄ (M+H)⁺: m/z=530.2; found: 530.2.

### Example 63. Methyl ((1R,3R)-3-(6-((4-(cyclobutoxymethyl)-6-(2,3-dihydrobenzo[b][1,4]dioxin-6-yl)pyridin-2-yl)amino)-3-(methyl-d3)-2-oxo-2,3-dihydro-1H-imidazo[4,5-c]pyridin-1-yl)cyclopentyl)carbamate

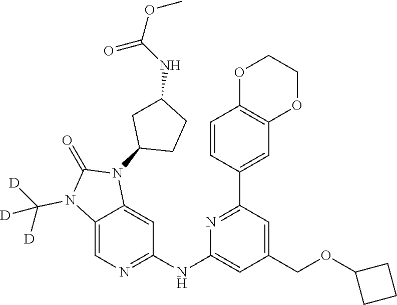 [SMILES: [2H]C([2H])([2H])n1c(=O)n([C@@H]2CC[C@@H](NC(=O)OC)C2)c2cc(Nc3cc(COC4CCC4)cc(-c4ccc5c(c4)OCCO5)n3)ncc21]

### Step 1. Methyl 2-amino-6-(2,3-dihydrobenzo[b][1,4]dioxin-6-yl)isonicotinate

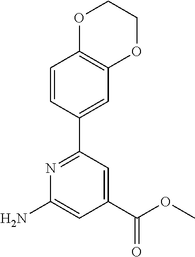 [SMILES: COC(=O)c1cc(N)nc(-c2ccc3c(c2)OCCO3)c1]

To a reaction vial containing methyl 2-amino-6-chloroisonicotinate (Ambeed, 1.09 g, 5.83 mmol), (2,3-dihydrobenzo[b][1,4]dioxin-6-yl)boronic acid (1.5 g, 8.33 mmol), [1,1′-bis(diphenylphosphino)ferrocene]dichloropalladium(II)-dichloromethane complex (681 mg, 0.83 mmol) and sodium carbonate (2.65 g, 25 mmol) was added dioxane (20 mL) and water (4 mL). The reaction mixture was stirred at 80° C. for 2 h. The reaction mixture was allowed to cool to r.t and extracted with EtOAc. The organic layers were combined, dried over sodium sulfate, filtered, and the solvents were evaporated in vacuo. The crude material was purified by Biotage Isolera to give the product. LCMS calculated for C₁₅H₁₅N₂O₄(M+H)⁺: m/z=287.1; found 287.1.

### Step 2. 2-(2,3-Dihydrobenzo[b][1.4]dioxin-6-yl)-6-((1-((1R,3R)-3-((methoxycarbonyl)amino)cyclopentyl)-3-(methyl-d3)-2-oxo-2,3-dihydro-1H-imidazo[4,5-c]pyridin-6-yl)amino)isonicotinic acid

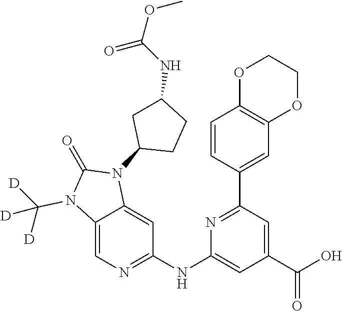 [SMILES: [2H]C([2H])([2H])n1c(=O)n([C@@H]2CC[C@@H](NC(=O)OC)C2)c2cc(Nc3cc(C(=O)O)cc(-c4ccc5c(c4)OCCO5)n3)ncc21]

A reaction vial containing methyl ((1R,3R)-3-(6-chloro-3-(methyl-d₃)-2-oxo-2,3-dihydro-1H-imidazo[4,5-c]pyridin-1-yl)cyclopentyl)carbamate (Example 75, Step 1, 1.33 g, 4.09 mmol), methyl 2-amino-6-(2,3-dihydrobenzo[b][1,4]dioxin-6-yl)isonicotinate (1.17 g, 4.09 mmol), [(2-di-tert-butylphosphino-3,6-dimethoxy-2′,4′,6′-triisopropyl-1,1′-biphenyl)-2-(2′-amino-1,1′-biphenyl)]palladium(II) methanesulfonate (698 mg, 0.82 mmol) and sodium tert-butoxide (1.18 g, 12.26 mmol) was evacuated and backfilled with nitrogen. THF (20 mL) was added to the reaction mixture, which was then stirred at 50° C. for 2 h. The reaction mixture was cooled to r.t and diluted with EtOAc. The organic layer was subsequently washed with water and brine, dried over sodium sulfate, filtered, and the solvents were evaporated in vacuo. The crude material was purified by Biotage Isolera. LCMS calculated for C₂₈H₂₆D₃N₆O₇ (M+H)⁺: m/z=564.2; found 564.2.

### Step 3. Methyl ((1R,3R)-3-(6-((6-(2,3-dihydrobenzo[b][1.4]dioxin-6-yl)-4-(hydroxymethyl)pyridin-2-yl)amino)-3-(methyl-d3)-2-oxo-2,3-dihydro-1H-imidazo[4,5-c]pyridin-1-yl)cyclopentyl)carbamate

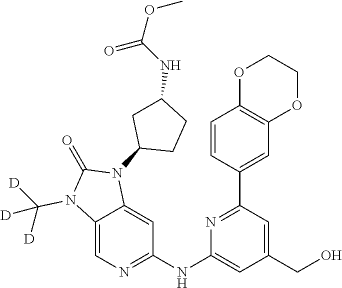 [SMILES: [2H]C([2H])([2H])n1c(=O)n([C@@H]2CC[C@@H](NC(=O)OC)C2)c2cc(Nc3cc(CO)cc(-c4ccc5c(c4)OCCO5)n3)ncc21]

To a cooled reaction vial containing 2-(2,3-dihydrobenzo[b][1,4]dioxin-6-yl)-6-((1-((1R,3R)-3-((methoxycarbonyl)amino)cyclopentyl)-3-(methyl-d₃)-2-oxo-2,3-dihydro-1H-imidazo[4,5-c]pyridin-6-yl)amino)isonicotinic acid (300 mg, 0.53 mmol), triethylamine (161 mg, 1.60 mmol) and THF (3 mL) was added isobutyl chloroformate (145 mg, 1.06 mmol) dropwise, which was then stirred at 0° C. for 20 min. The reaction mixture was filtered and the resulting solution was collected. To the cooled solution was added NaBH₄ (202 mg, 5.32 mmol) in water (1 mL) dropwise and the mixture was stirred at 0° C. for 10 min before warmed to r.t and diluted with EtOAc. The organic layer was subsequently washed with water and brine, dried over sodium sulfate, filtered, and the solvents were evaporated in vacuo. The crude material was used directly for next step without further purification. LCMS calculated for C₂₈H₂₈D₃N₆O₆ (M+H)⁺: m/z=550.2; found 550.2.

### Step 4. (2-(2,3-Dihydrobenzo[b][1.4]dioxin-6-yl)-6-((1-((1R,3R)-3-((methoxycarbonyl)amino)cyclopentyl)-3-(methyl-d3)-2-oxo-2,3-dihydro-1H-imidazo[4,5-c]pyridin-6-yl)amino)pyridin-4-yl)methyl methanesulfonate

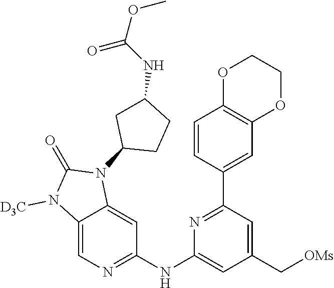 [SMILES: [2H]C([2H])([2H])n1c(=O)n([C@@H]2CC[C@@H](NC(=O)OC)C2)c2cc(Nc3cc(COS(C)(=O)=O)cc(-c4ccc5c(c4)OCCO5)n3)ncc21]

To a cooled reaction vial containing methyl ((1R,3R)-3-(6-((6-(2,3-dihydrobenzo[b][1,4]dioxin-6-yl)-4-(hydroxymethyl)pyridin-2-yl)amino)-3-(methyl-d₃)-2-oxo-2,3-dihydro-1H-imidazo[4,5-c]pyridin-1-yl)cyclopentyl)carbamate (350 mg, 0.64 mmol), triethylamine (193 mg, 1.91 mmol) and DCM (7 mL) was added methanesulfonyl chloride (146 mg, 1.27 mmol) dropwise, which was then stirred at 0° C. for 30 min. The reaction mixture was warmed to r.t and diluted with DCM. The organic layer was subsequently washed with water and brine, dried over sodium sulfate, filtered, and the solvents were evaporated in vacuo. The crude material was used directly for next step. LCMS calculated for C₂₉H₃₀D₃N₆O₈S (M+H)⁺: m/z=628.2; found 628.2.

### Step 5. Methyl ((1R,3R)-3-(6-((4-(cyclobutoxymethyl)-6-(2,3-dihydrobenzo[b][1.4]dioxin-6-yl)pyridin-2-yl)amino)-3-(methyl-d3)-2-oxo-2,3-dihydro-1H-imidazo[4,5-c]pyridin-1-yl)cyclopentyl)carbamate

To a reaction vial containing (2-(2,3-dihydrobenzo[b][1,4]dioxin-6-yl)-6-((1-((1R,3R)-3-((methoxycarbonyl)amino)cyclopentyl)-3-(methyl-d₃)-2-oxo-2,3-dihydro-1H-imidazo[4,5-c]pyridin-6-yl)amino)pyridin-4-yl)methyl methanesulfonate (15 mg, 0.024 mmol), cesium carbonate (23 mg, 0.072 mmol) and MeCN (0.5 mL) was added cyclobutanol (17 mg, 0.24 mmol), which was then stirred at 70° C. for 2 h. The reaction mixture was cooled to r.t, diluted with MeCN and purified by prep-LCMS (XBridge C18 column, eluting with a gradient of acetonitrile/water containing 0.1% TFA, at flow rate of 60 mL/min. LCMS calculated for C₃₂H₃₄D₃N₆O₆ (M+H)⁺: m/z=604.3; found: 604.3.

### Example 64. Methyl ((1R,3R)-3-(6-((6-(3-aminopyrrolidin-1-yl)pyridin-2-yl)amino)-3-methyl-2-oxo-2,3-dihydro-1H-imidazo[4,5-c]pyridin-1-yl)cyclopentyl)carbamate

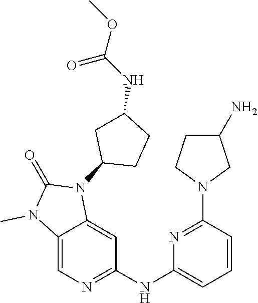 [SMILES: COC(=O)N[C@@H]1CC[C@@H](n2c(=O)n(C)c3cnc(Nc4cccc(N5CCC(N)C5)n4)cc32)C1]

### Step 1. 6-(3-Aminopyrrolidin-1-yl)pyridin-2-amine

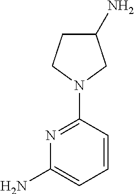 [SMILES: Nc1cccc(N2CCC(N)C2)n1]

To a vial containing 6-fluoropyridin-2-amine (100 mg, 0.892 mmol) and potassium carbonate (247 mg, 1.784 mmol) in DMSO (0.2 mL) was added tert-butyl pyrrolidin-3-ylcarbamate (332 mg, 1.784 mmol). The reaction mixture was heated at 100° C. for 16 h. After cooling to room temperature, the reaction mixture was diluted with CH₂Cl₂ (5 mL), filtered through a celite pad and washed with CH₂Cl₂ (15 mL) for 3 times. The filtrate was concentrated in vacuo and was washed with aqueous saturated sodium bicarbonate solution. The organic phase was washed with brine, dried over MgSO₄ and concentrated in vacuo. The crude material was redissolved in CH₂Cl₂ (0.2 ml) and TFA (1.0 ml). After stirring at 30° C. for 1 h, the reaction mixture was concentrated in vacuo. The crude material was redissolved in CH₂Cl₂ (5 mL) and the pH of the mixture was adjusted to ˜10 with ammonia aqueous solution and then extracted into CH₂Cl₂. The organic phase was washed with brine, dried over MgSO₄ and concentrated in vacuo. The obtained crude product was then diluted with acetonitrile and purified with prep-LCMS (XBridge C18 column, eluting with a gradient of acetonitrile/water containing 0.1% TFA, at flow rate of 60 mL/min). LCMS calculated for C₉H₁₅N4 (M+H)⁺: m/z=179.1; found 179.1.

### Step 2. Methyl (1R,3R)-3-(6-(6-(3-aminopyrrolidin-1-yl)pyridin-2-yl)amino)-3-methyl-2-oxo-2,3-dihydro-1H-imidazo[4,5-c]pyridin-1-yl)cyclopentyl)carbamate

The title compound was prepared according to the procedures described in Example 53, with 6-(3-aminopyrrolidin-1-yl)pyridin-2-amine replacing methyl (3-amino-5-(1-methyl-1H-pyrazol-4-yl)phenyl)carbamate in Step 4. LCMS calculated for C₂₃H₃₁N₈O₃ (M+H)⁺: m/z=467.2; found: 467.2. ¹H NMR (400 MHZ, DMSO) δ 11.02 (bs, 1H), 8.29 (s, 1H), 8.20-8.15 (m, 2H), 7.66 (t, J=8.1 Hz, 1H), 7.39 (d, J=6.8 Hz, 1H), 7.09 (s, 1H), 6.40 (d, J=7.8 Hz, 1H), 6.26 (d, J=8.3 Hz, 1H), 4.93 (p, J=8.7 Hz, 1H), 4.21 (m, 1H), 4.04 (m, 1H), 3.85-3.68 (m, 3H), 3.68-3.63 (m, 1H), 3.55 (s, 3H), 3.36 (s, 3H), 2.41 (m, 1H), 2.35-2.23 (m, 1H), 2.23-2.07 (m, 3H), 2.09-1.98 (m, 1H), 1.93 (m, 1H), 1.65 (m, 1H).

### Example 65. Methyl ((1R,3R)-3-(6-((4-((cyclopentylamino)methyl)-6-(2,3-dihydrobenzo[b][1,4]dioxin-6-yl)pyridin-2-yl)amino)-3-(methyl-d₃)-2-oxo-2,3-dihydro-1H-imidazo[4,5-c]pyridin-1-yl)cyclopentyl)carbamate

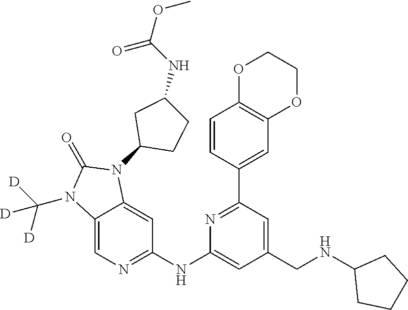 [SMILES: [2H]C([2H])([2H])n1c(=O)n([C@@H]2CC[C@@H](NC(=O)OC)C2)c2cc(Nc3cc(CNC4CCCC4)cc(-c4ccc5c(c4)OCCO5)n3)ncc21]

The title compound was prepared according to the procedures described in Example 63, with cyclopentanamine replacing cyclobutanol in Step 5. LCMS calculated for C₃₃H₃₇D₃N₇O₅ (M+H)⁺: m/z=617.3; found 617.3. ¹H NMR (400 MHZ, DMSO-d₆) δ 10.15 (s, 1H), 8.97 (s, 1H), 8.14 (s, 1H), 7.63 (s, 1H), 7.59 (d, J=2.1 Hz, 1H), 7.56 (d, J=2.2 Hz, 1H), 7.54 (d, J=2.2 Hz, 1H), 7.40 (s, 1H), 7.33 (s, 1H), 7.02 (d, J=8.4 Hz, 1H), 4.89 (q, J=8.5 Hz, 1H), 4.32 (s, 4H), 4.21 (t, J=6.1 Hz, 2H), 4.13 (d, J=7.3 Hz, 1H), 3.60 (d, J=9.0 Hz, 1H), 3.53 (s, 3H), 2.27 (m, 1H), 2.15-1.95 (m, 5H), 1.96-1.88 (m, 1H), 1.72 (m, 4H), 1.58 (d, J=8.3 Hz, 2H), 1.25 (s, 1H).

### Example 66. Methyl ((1R,3R)-3-(3-methyl-2-oxo-6-((2-(piperidin-1-yl)pyrimidin-4-yl)amino)-2,3-dihydro-1H-imidazo[4,5-c]pyridin-1-yl)cyclopentyl)carbamate

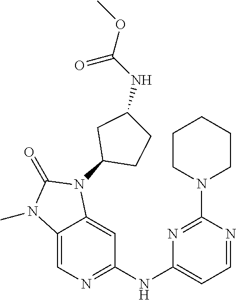 [SMILES: COC(=O)N[C@@H]1CC[C@@H](n2c(=O)n(C)c3cnc(Nc4ccnc(N5CCCCC5)n4)cc32)C1]

### Step 1. 2-(Piperidin-1-yl)pyrimidin-4-amine

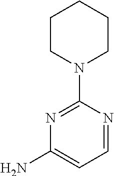 [SMILES: Nc1ccnc(N2CCCCC2)n1]

To a vial containing 2-chloropyrimidin-4-amine (100 mg, 0.772 mmol) and DIPEA (270 μL, 1.544 mmol) in DMSO (0.1 mL) was added piperidine (131 mg, 1.544 mmol). The reaction mixture was heated at 90° C. for 16 h. After cooling to room temperature, the reaction mixture was diluted with CH₂Cl₂ (5 mL), washed with aqueous saturated sodium bicarbonate solution and then extracted into CH₂Cl₂. The organic phase was washed with brine, dried over MgSO₄ and concentrated in vacuo. The obtained crude product was then diluted with acetonitrile and purified with prep-LCMS (XBridge C18 column, eluting with a gradient of acetonitrile/water containing 0.1% TFA, at flow rate of 60 mL/min). LCMS calculated for C₉H₁₅N₄ (M+H)⁺: m/z=179.1; found 179.1.

### Step 2. Methyl ((1R,3R)-3-(3-methyl-2-oxo-6-((2-(piperidin-1-yl)pyrimidin-4-yl)amino)-2,3-dihydro-1H-imidazo[4,5-c]pyridin-1-yl)cyclopentyl)carbamate

The title compound was prepared according to the procedures described in Example 53, with 2-(piperidin-1-yl)pyrimidin-4-amine replacing methyl (3-amino-5-(1-methyl-1H-pyrazol-4-yl)phenyl)carbamate in Step 4. LCMS calculated for C₂₃H₃₁N₈O₃ (M+H)⁺: m/z=467.2; found: 467.2. ¹H NMR (400 MHZ, DMSO) δ 10.98 (s, 1H), 8.22 (s, 1H), 7.96 (d, J=7.0 Hz, 1H), 7.72 (bs, 1H), 7.35 (d, J=7.0 Hz, 1H), 4.88-4.79 (m, 1H), 4.22-4.14 (m, 1H), 3.72 (m, 4H), 3.55 (s, 3H), 3.37 (s, 3H), 2.30 (m, 1H), 2.16 (m, 1H), 2.04 (m, 2H), 1.88 (ddd, J=13.7, 9.2, 4.8 Hz, 1H), 1.74-1.51 (m, 7H).

### Example 67. Methyl ((1R,3R)-3-(6-((2′-methoxy-4-methyl-[2,4′-bipyridin]-6-yl)amino)-3-methyl-2-oxo-2,3-dihydro-1H-imidazo[4,5-c]pyridin-1-yl)cyclopentyl)carbamate

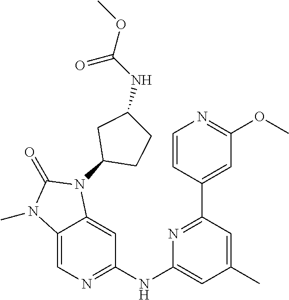

### Step 1. 2′-Methoxy-4-methyl-[2,4′-bipyridin]-6-amine

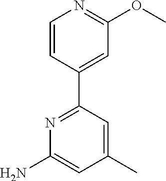 [SMILES: COc1cc(-c2cc(C)cc(N)n2)ccn1]

A vial containing (2-methoxypyridin-4-yl)boronic acid (32 mg, 0.210 mmol), 6-chloro-4-methylpyridin-2-amine (25 mg, 0.175 mmol), potassium carbonate (49 mg, 0.351 mmol) and (1,1′-bis(diphenylphosphino)ferrocene)-dichloropalladium(II) dichloromethane adduct (14 mg, 0.018 mmol) was evacuated and backfilled with nitrogen three times, followed by the addition of 1,4-dioxane (0.46 mL) and water (46 μL). The vial was sealed and heated to 100° C. for 4 h. After cooling to room temperature, the mixture was filtered through a SiliaPrep SPE thiol cartridge (SPE-R51030B-06P) and washed with acetonitrile. The mixture was then diluted with acetonitrile and purified with prep-LCMS (XBridge C18 column, eluting with a gradient of acetonitrile/water containing 0.1% TFA, at flow rate of 60 mL/min). LCMS calculated for C₁₂H₁₄N₃O (M+H)⁺: m/z=216.1; found 216.1.

### Step 2. Methyl ((1R,3R)-3-(6-((2′-methoxy-4-methyl-[2,4′-bipyridin]-6-yl)amino)-3-methyl-2-oxo-2,3-dihydro-1H-imidazo[4,5-c]pyridin-1-yl)cyclopentyl)carbamate

The title compound was prepared according to the procedures described in Example 53, with 2′-methoxy-4-methyl-[2,4′-bipyridin]-6-amine replacing methyl (3-amino-5-(1-methyl-1H-pyrazol-4-yl)phenyl)carbamate in Step 4. LCMS calculated for C₂₆H₃₀N₇O₄ (M+H)⁺: m/z=504.2; found: 504.2. ¹H NMR (400 MHz, DMSO) δ 10.81 (bs, 1H), 8.32 (d, J=5.4 Hz, 1H), 8.29 (s, 1H), 7.66-7.57 (m, 2H), 7.44-7.32 (m, 3H), 7.11 (s, 1H), 4.94 (p, J=8.5 Hz, 1H), 4.17 (m, 1H), 3.93 (s, 3H), 3.54 (s, 3H), 3.38 (s, 3H), 2.44 (s, 3H), 2.29 (dt, J=13.6, 7.9 Hz, 1H), 2.19-1.87 (m, 4H), 1.58 (m, 1H).

### Example 68. Methyl ((1R,3R)-3-(3-methyl-2-oxo-6-((4-(pyridin-4-yl)thiazol-2-yl)amino)-2,3-dihydro-1H-imidazo[4,5-c]pyridin-1-yl)cyclopentyl)carbamate

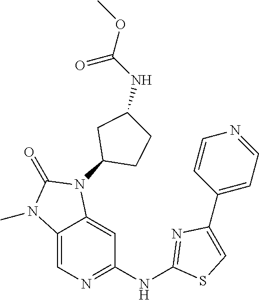 [SMILES: COC(=O)N[C@@H]1CC[C@@H](n2c(=O)n(C)c3cnc(Nc4nc(-c5ccncc5)cs4)cc32)C1]

The title compound was prepared according to the procedures described in Example 53, with 4-(pyridin-4-yl)thiazol-2-amine replacing methyl (3-amino-5-(1-methyl-1H-pyrazol-4-yl)phenyl)carbamate in Step 4. LCMS calculated for C₂₂H₂₄N₇O₃S (M+H)⁺: m/z=466.2; found: 466.2. ¹H NMR (600 MHz, DMSO) δ 11.40 (s, 1H), 8.86 (d, J=6.5 Hz, 2H), 8.30 (d, J=6.5 Hz, 2H), 8.21 (s, 1H), 8.13 (s, 1H), 7.40 (d, J=7.1 Hz, 1H), 6.96 (s, 1H), 4.92 (p, J=8.7 Hz, 1H), 4.23 (m, 1H), 3.55 (s, 3H), 3.35 (s, 3H), 2.30-2.18 (m, 2H), 2.10 (m, 1H), 2.03-1.87 (m, 2H), 1.64 (m, 1H).

### Example 69. Methyl ((1R,3R)-3-(6-((3-fluoro-6′-(trifluoromethyl)-[2,3′-bipyridin]-6-yl)amino)-3-methyl-2-oxo-2,3-dihydro-1H-imidazo[4,5-c]pyridin-1-yl)cyclopentyl)carbamate

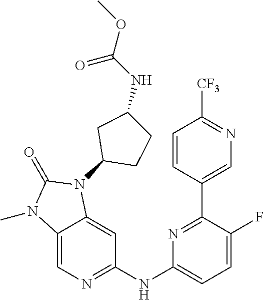 [SMILES: COC(=O)N[C@@H]1CC[C@@H](n2c(=O)n(C)c3cnc(Nc4ccc(F)c(-c5ccc(C(F)(F)F)nc5)n4)cc32)C1]

### Step 1. 3-Fluoro-6′-(trifluoromethyl)-[2,3′-bipyridin]-6-amine

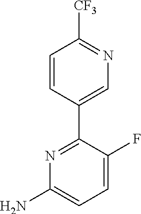 [SMILES: Nc1ccc(F)c(-c2ccc(C(F)(F)F)nc2)n1]

A vial containing (6-(trifluoromethyl)pyridin-3-yl)boronic acid (24 mg, 0.126 mmol), 6-bromo-5-fluoropyridin-2-amine (20 mg, 0.105 mmol), potassium carbonate (29 mg, 0.209 mmol) and (1,1′-bis(diphenylphosphino)ferrocene)-dichloropalladium(II) dichloromethane adduct (9 mg, 10.47 μmol) was evacuated and backfilled with nitrogen three times, followed by the addition of 1,4-dioxane (0.46 mL) and water (46 μL). The vial was sealed and heated to 100° C. for 4 h. After cooling to room temperature, the mixture was filtered through a SiliaPrep SPE thiol cartridge (SPE-R51030B-06P) and washed with acetonitrile. The mixture was then diluted with acetonitrile and purified with prep-LCMS (XBridge C18 column, eluting with a gradient of acetonitrile/water containing 0.1% TFA, at flow rate of 60 mL/min). LCMS calculated for C₁₁H₈F₄N₃ (M+H)⁺: m/z=258.1; found 258.1.

### Step 2. Methyl ((1R,3R)-3-(6-((3-fluoro-6′-(trifluoromethyl)-[2,3′-bipyridin]-6-yl)amino)-3-methyl-2-oxo-2,3-dihydro-1H-imidazo[4,5-c]pyridin-1-yl)cyclopentyl)carbamate

The title compound was prepared according to the procedures described in Example 53, with 3-fluoro-6′-(trifluoromethyl)-[2,3′-bipyridin]-6-amine replacing methyl (3-amino-5-(1-methyl-1H-pyrazol-4-yl)phenyl)carbamate in Step 4. LCMS calculated for C₂₅H₂₄F₄N₇O₃ (M+H)⁺: m/z=546.2; found: 546.2. ¹H NMR (400 MHZ, DMSO) δ 10.61 (bs, 1H), 9.27 (s, 1H), 8.60-8.53 (m, 1H), 8.18 (s, 1H), 8.08 (d, J=8.2 Hz, 1H), 7.95 (t, J=9.7 Hz, 1H), 7.52 (d, J=9.0 Hz, 1H), 7.43 (s, 1H), 7.30 (d, J=7.2 Hz, 1H), 4.92 (p, J=8.5 Hz, 1H), 4.07 (m, 1H), 3.53 (s, 3H), 3.36 (s, 3H), 2.24 (dt, J=13.6, 8.0 Hz, 1H), 2.14-1.82 (m, 4H), 1.57-1.49 (m, 1H).

### Example 70. Methyl ((1R,3R)-3-(6-((6-(2,3-dihydrobenzo[b][1,4]dioxin-6-yl)-4-(((S)-3-fluoropyrrolidin-1-yl)methyl)pyridin-2-yl)amino)-3-(methyl-d₃)-2-oxo-2,3-dihydro-1H-imidazo[4,5-c]pyridin-1-yl)cyclopentyl)carbamate

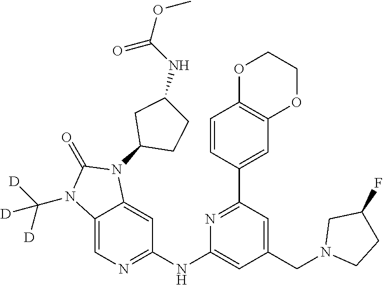 [SMILES: [2H]C([2H])([2H])n1c(=O)n([C@@H]2CC[C@@H](NC(=O)OC)C2)c2cc(Nc3cc(CN4CC[C@H](F)C4)cc(-c4ccc5c(c4)OCCO5)n3)ncc21]

The title compound was prepared according to the procedures described in Example 63, with (S)-3-fluoropyrrolidine hydrochloride replacing cyclobutanol in Step 5. LCMS calculated for C₃₂H₃₄D₃FN₇O₅ (M+H)⁺: m/z=621.3; found 621.3. ¹H NMR (400 MHZ, DMSO-d₆) δ 10.19 (s, 1H), 8.15 (s, 1H), 7.63 (s, 1H), 7.59 (d, J=2.1 Hz, 1H), 7.57-7.51 (m, 2H), 7.39 (s, 1H), 7.33 (d, J=6.5 Hz, 1H), 7.02 (d, J=8.4 Hz, 1H), 5.49 (d, J=52.9 Hz, 1H), 4.91 (t, J=8.4 Hz, 1H), 4.45 (s, 1H), 4.32 (s, 5H), 4.13 (s, 1H), 3.91-3.73 (m, 1H), 3.71-3.56 (m, 1H), 3.53 (s, 3H), 3.45-3.23 (m, 2H), 2.27 (m, 2H), 2.15-1.97 (m, 3H), 1.90 (m, 1H), 1.56 (s, 1H), 1.25 (s, 1H).

### Example 71. Methyl ((1R,3R)-3-(6-((6-(4-(cyanomethyl)phenyl)-5-fluoropyridin-2-yl)amino)-3-methyl-2-oxo-2,3-dihydro-1H-imidazo[4,5-c]pyridin-1-yl)cyclopentyl)carbamate

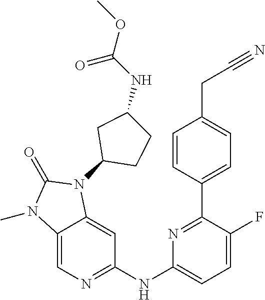 [SMILES: COC(=O)N[C@@H]1CC[C@@H](n2c(=O)n(C)c3cnc(Nc4ccc(F)c(-c5ccc(CC#N)cc5)n4)cc32)C1]

The title compound was prepared according to the procedures described in Example 69, with (4-(cyanomethyl)phenyl)boronic acid replacing (6-(trifluoromethyl)pyridin-3-yl)boronic acid in Step 1. LCMS calculated for C₂₇H₂₇FN₇O₃ (M+H)⁺: m/z=516.2; found: 516.2. ¹H NMR (400 MHZ, DMSO) δ 10.63 (s, 1H), 8.22 (s, 1H), 7.95 (dd, J=8.3, 1.5 Hz, 2H), 7.88 (t, J=9.7 Hz, 1H), 7.54 (d, J=8.2 Hz, 2H), 7.44-7.28 (m, 3H), 4.92 (p, J=8.4 Hz, 1H), 4.16 (s, 2H), 4.12-4.03 (m, 1H), 3.54 (s, 3H), 3.36 (s, 3H), 2.25 (dt, J=13.5, 8.0 Hz, 1H), 2.14-1.83 (m, 4H), 1.54 (m, 1H).

### Example 72. Methyl ((1R,3R)-3-(6-((6-(4-cyclopropylphenyl)-5-fluoropyridin-2-yl)amino)-3-methyl-2-oxo-2,3-dihydro-1H-imidazo[4,5-c]pyridin-1-yl)cyclopentyl)carbamate

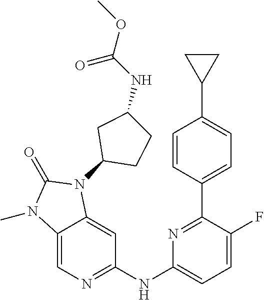 [SMILES: COC(=O)N[C@@H]1CC[C@@H](n2c(=O)n(C)c3cnc(Nc4ccc(F)c(-c5ccc(C6CC6)cc5)n4)cc32)C1]

The title compound was prepared according to the procedures described in Example 69, with (4-cyclopropylphenyl)boronic acid replacing (6-(trifluoromethyl)pyridin-3-yl)boronic acid in Step 1. LCMS calculated for C₂₈H₃₀FN₆O₃ (M+H)⁺: m/z=517.2; found: 517.2.

### Example 73. Methyl ((1R,3R)-3-(6-((5-fluoro-6-(4-(trifluoromethoxy)phenyl)pyridin-2-yl)amino)-3-methyl-2-oxo-2,3-dihydro-1H-imidazo[4,5-c]pyridin-1-yl)cyclopentyl)carbamate

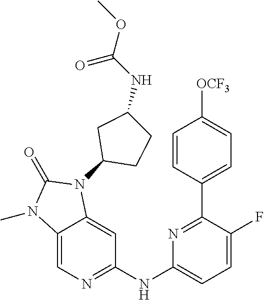 [SMILES: COC(=O)N[C@@H]1CC[C@@H](n2c(=O)n(C)c3cnc(Nc4ccc(F)c(-c5ccc(OC(F)(F)F)cc5)n4)cc32)C1]

The title compound was prepared according to the procedures described in Example 69, with (4-(trifluoromethoxy)phenyl)boronic acid replacing (6-(trifluoromethyl)pyridin-3-yl)boronic acid in Step 1. LCMS calculated for C₂₆H₂₅F₄N₆O₄ (M+H)⁺: m/z=561.2; found: 561.2. ¹H NMR (400 MHZ, DMSO) δ 10.55 (bs, 1H), 8.19 (s, 1H), 8.05 (d, J=7.4 Hz, 2H), 7.89 (m, 1H), 7.54 (d, J=8.4 Hz, 2H), 7.46-7.29 (m, 3H), 4.92 (p, J=8.4 Hz, 1H), 4.10 (m, 1H), 3.53 (s, 3H), 3.36 (s, 3H), 2.25 (dt, J=13.6, 8.0 Hz, 1H), 2.13-1.83 (m, 4H), 1.54 (s, 1H).

### Example 74. Methyl ((1R,3R)-3-(6-((6-(2,3-dihydrobenzo[b][1,4]dioxin-6-yl)-4-(morpholinomethyl)pyridin-2-yl)amino)-3-(methyl-d₃)-2-oxo-2,3-dihydro-1H-imidazo[4,5-c]pyridin-1-yl)cyclopentyl)carbamate

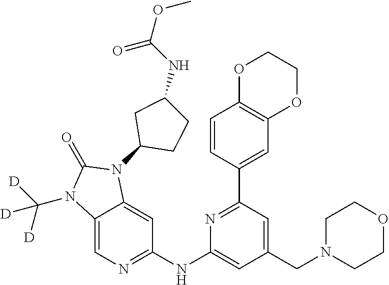 [SMILES: [2H]C([2H])([2H])n1c(=O)n([C@@H]2CC[C@@H](NC(=O)OC)C2)c2cc(Nc3cc(CN4CCOCC4)cc(-c4ccc5c(c4)OCCO5)n3)ncc21]

The title compound was prepared according to the procedures described in Example 63, with morpholine replacing cyclobutanol in Step 5. LCMS calculated for C₃₂H₃₅D₃N₇O₆ (M+H)⁺: m/z=619.3; found 619.3. ¹H NMR (400 MHZ, DMSO-d₆) δ 10.19 (s, 1H), 8.17 (s, 1H), 7.57 (d, J=2.1 Hz, 1H), 7.53 (dd, J=8.4, 2.2 Hz, 1H), 7.50 (s, 2H), 7.33 (d, J=7.7 Hz, 2H), 7.02 (d, J=8.4 Hz, 1H), 4.96-4.87 (m, 1H), 4.32 (s, 6H), 4.19-4.10 (m, 1H), 3.96 (s, 2H), 3.70 (s, 2H), 3.53 (s, 3H), 3.39-3.03 (m, 4H), 2.27 (m, 1H), 2.17-1.97 (m, 3H), 1.90 (m, 1H), 1.57 (s, 1H).

### Example 75. Methyl ((1R,3R)-3-(6-((2-(2-methoxypyridin-4-yl)pyrimidin-4-yl)amino)-3-(methyl-d₃)-2-oxo-2,3-dihydro-1H-imidazo[4,5-c]pyridin-1-yl)cyclopentyl)carbamate

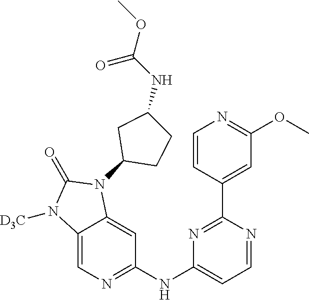 [SMILES: [2H]C([2H])([2H])n1c(=O)n([C@@H]2CC[C@@H](NC(=O)OC)C2)c2cc(Nc3ccnc(-c4ccnc(OC)c4)n3)ncc21]

### Step 1. Methyl ((1R,3R)-3-(6-chloro-3-(methyl-d3)-2-oxo-2,3-dihydro-1H-imidazo[4,5-c]pyridin-1-yl)cyclopentyl)carbamate

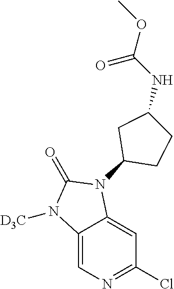 [SMILES: [2H]C([2H])([2H])n1c(=O)n([C@@H]2CC[C@@H](NC(=O)OC)C2)c2cc(Cl)ncc21]

The title compound was prepared according to the procedures described in Intermediate A, with iodomethane-d₃ replacing iodomethane in Step 4. LCMS calculated for C₁₄H₁₅D₃ClN₄O₃ (M+H)⁺: m/z=328.1; found: 328.1.

### Step 2. 2-(2-Methoxypyridin-4-yl)pyrimidin-4-amine

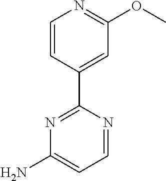

The title compound was prepared according to the procedures described in Example 67, with 2-chloropyrimidin-4-amine replacing 6-chloro-4-methylpyridin-2-amine in Step 1. LCMS calculated for C₁₀H₁₁N₄O (M+H)⁺: m/z=203.1; found 203.1.

### Step 3. Methyl ((1R,3R)-3-(6-((2-(2-methoxypyridin-4-yl)pyrimidin-4-yl)amino)-3-(methyl-d₃)-2-oxo-2,3-dihydro-1H-imidazo[4,5-c]pyridin-1-yl)cyclopentyl)carbamate

The title compound was prepared according to the procedures described in Example 53, with methyl ((1R,3R)-3-(6-chloro-3-(methyl-d₃)-2-oxo-2,3-dihydro-1H-imidazo[4,5-c]pyridin-1-yl)cyclopentyl)carbamate replacing methyl ((1R,3R)-3-(6-chloro-3-methyl-2-oxo-2,3-dihydro-1H-imidazo[4,5-c]pyridin-1-yl)cyclopentyl)carbamate and 2-(2-methoxypyridin-4-yl)pyrimidin-4-amine replacing methyl (3-amino-5-(1-methyl-1H-pyrazol-4-yl)phenyl)carbamate in Step 4. LCMS calculated for C₂₄H₂₄D₃N₈O₄ (M+H)⁺: m/z=494.2; found: 494.2. ¹H NMR (400 MHZ, DMSO) δ 10.34 (s, 1H), 8.56 (d, J=5.9 Hz, 1H), 8.33 (d, J=5.3 Hz, 1H), 8.17 (s, 1H), 7.81 (dd, J=5.4, 1.4 Hz, 1H), 7.69 (m, 1H), 7.61 (s, 1H), 7.54 (m, 1H), 7.33 (m, 1H), 4.92 (p, J=8.5 Hz, 1H), 4.17 (m, 1H), 3.92 (s, 3H), 3.52 (s, 3H), 2.38-2.26 (m, 1H), 2.21-2.00 (m, 3H), 1.92 (m, 1H), 1.58 (m, 1H).

### Example 76. Methyl ((1R,3R)-3-(6-((6-(2,3-dihydrobenzo[b][1,4]dioxin-6-yl)-4-(octahydro-2H-pyrido[1,2-a]pyrazine-2-carbonyl)pyridin-2-yl)amino)-3-(methyl-d₃)-2-oxo-2,3-dihydro-1H-imidazo[4,5-c]pyridin-1-yl)cyclopentyl)carbamate

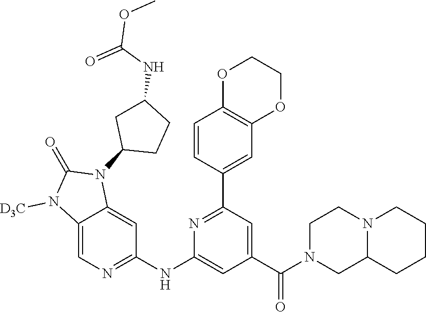 [SMILES: [2H]C([2H])([2H])n1c(=O)n([C@@H]2CC[C@@H](NC(=O)OC)C2)c2cc(Nc3cc(C(=O)N4CCN5CCCCC5C4)cc(-c4ccc5c(c4)OCCO5)n3)ncc21]

A mixture of 2-(2,3-dihydrobenzo[b][1,4]dioxin-6-yl)-6-((1-((1R,3R)-3-((methoxycarbonyl)amino)cyclopentyl)-3-(methyl-d₃)-2-oxo-2,3-dihydro-1H-imidazo[4,5-c]pyridin-6-yl)amino)isonicotinic acid (Example 63, Step 2, 10 mg, 0.018 mmol), octahydro-2H-pyrido[1,2-a]pyrazine (5 mg, 0.035 mmol), benzotriazol-1-yloxytris(dimethylamino)-phosphonium hexafluorophosphate (12 mg, 0.027 mmol) and DIPEA (5 μl, 0.027 mmol) in DMF (0.5 ml) was stirred at r.t. for 10 min. The mixture was diluted with CH₃CN and water and purified with prep-LCMS (XBridge C18 column, eluting with a gradient of acetonitrile/water containing 0.1% TFA, at flow rate of 60 mL/min). LCMS calculated for C₃₆H₄₀D₃N₈O₆ (M+H)⁺: m/z=686.3; found 686.3. ¹H NMR (400 MHZ, DMSO-d₆) δ 10.22 (s, 1H), 8.15 (s, 1H), 7.66 (s, 1H), 7.60 (d, J=2.1 Hz, 1H), 7.57 (dd, J=8.3, 2.2 Hz, 1H), 7.35 (dd, J=17.8, 8.3 Hz, 3H), 7.00 (d, J=8.4 Hz, 1H), 4.91 (p, J=8.3 Hz, 1H), 4.74-4.58 (m, 1H), 4.32 (d, J=1.9 Hz, 4H), 4.13 (s, 1H), 3.88-3.71 (m, 1H), 3.53 (s, 3H), 3.49 (s, 2H), 3.31 (s, 2H), 3.18 (s, 1H), 2.94 (d, J=14.8 Hz, 1H), 2.28 (m, 1H), 2.20-1.93 (m, 4H), 1.96-1.79 (m, 3H), 1.78-1.61 (m, 2H), 1.58-1.20 (m, 3H).

### Example 77. Methyl ((1R,3R)-3-(6-((2-(5-azaspiro[2.4]heptan-5-yl)pyrimidin-4-yl)amino)-3-methyl-2-oxo-2,3-dihydro-1H-imidazo[4,5-c]pyridin-1-yl)cyclopentyl)carbamate

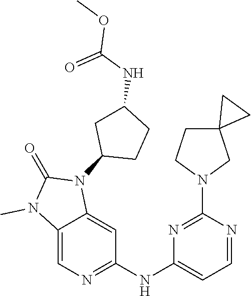 [SMILES: COC(=O)N[C@@H]1CC[C@@H](n2c(=O)n(C)c3cnc(Nc4ccnc(N5CCC6(CC6)C5)n4)cc32)C1]

The title compound was prepared according to the procedures described in Example 66, with 5-azaspiro[2.4]heptane replacing piperidine in Step 1. LCMS calculated for C₂₄H₃₁N₈O₃ (M+H)⁺: m/z=479.2; found: 479.2.

### Example 78. Methyl ((1R,3R)-3-(6-((2-(3,3-difluoropyrrolidin-1-yl)pyrimidin-4-yl)amino)-3-methyl-2-oxo-2,3-dihydro-1H-imidazo[4,5-c]pyridin-1-yl)cyclopentyl)carbamate

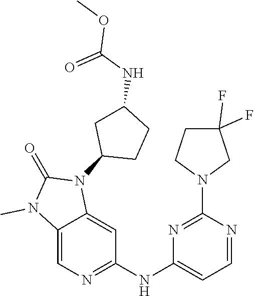 [SMILES: COC(=O)N[C@@H]1CC[C@@H](n2c(=O)n(C)c3cnc(Nc4ccnc(N5CCC(F)(F)C5)n4)cc32)C1]

The title compound was prepared according to the procedures described in Example 66, with 3,3-difluoropyrrolidine replacing piperidine in Step 1. LCMS calculated for C₂₂H₂₇F₂N₈O₃ (M+H)⁺: m/z=489.2; found: 489.2. ¹H NMR (600 MHZ, DMSO) δ 10.98 (s, 1H), 8.24 (s, 1H), 8.07 (d, J=6.7 Hz, 1H), 7.72 (bs, 1H), 7.36 (d, J=7.2 Hz, 1H), 6.72 (bs, 1H), 4.90 (p, J=8.8 Hz, 1H), 4.18 (m, 1H), 4.09-3.78 (m, 4H), 3.54 (s, 3H), 3.37 (s, 3H), 2.62 (m, 2H), 2.33 (m, 8.2 Hz, 1H), 2.18 (m, 1H), 2.10-1.97 (m, 2H), 1.87 (ddd, J=13.7, 9.1, 4.8 Hz, 1H), 1.60 (m, 1H).

### Example 79. Methyl ((1R,3R)-3-(3-methyl-2-oxo-6-((2-(4-(trifluoromethyl)piperidin-1-yl)pyrimidin-4-yl)amino)-2,3-dihydro-1H-imidazo[4,5-c]pyridin-1-yl)cyclopentyl)carbamate

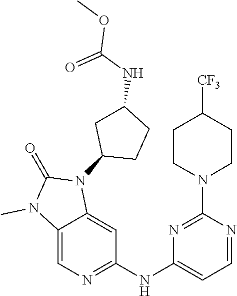 [SMILES: COC(=O)N[C@@H]1CC[C@@H](n2c(=O)n(C)c3cnc(Nc4ccnc(N5CCC(C(F)(F)F)CC5)n4)cc32)C1]

The title compound was prepared according to the procedures described in Example 66, with 4-(trifluoromethyl)piperidine replacing piperidine in Step 1. LCMS calculated for C₂₄H₃₀F₃N₈O₃ (M+H)⁺: m/z=535.2; found: 535.2. ¹H NMR (600 MHZ, DMSO) δ 10.98 (s, 1H), 8.23 (s, 1H), 8.01 (d, J=6.9 Hz, 1H), 7.59 (bs, 1H), 7.36 (d, J=7.1 Hz, 1H), 6.73 (bs, 1H), 4.88 (p, J=8.7 Hz, 1H), 4.48 (d, J=13.3 Hz, 2H), 4.18 (m, 1H), 3.54 (s, 3H), 3.37 (s, 3H), 3.17 (t, J=13.2 Hz, 2H), 2.77-2.69 (m, 1H), 2.30 (dt, J=13.4, 8.2 Hz, 1H), 2.18 (m, 1H), 2.10-1.82 (m, 5H), 1.64-1.50 (m, 3H).

### Example 80. Methyl ((1R,3R)-3-(6-((6-(2,3-dihydrobenzo[b][1,4]dioxin-6-yl)-4-((R)-3-fluoropyrrolidine-1-carbonyl)pyridin-2-yl)amino)-3-(methyl-d₃)-2-oxo-2,3-dihydro-1H-imidazo[4,5-c]pyridin-1-yl)cyclopentyl)carbamate

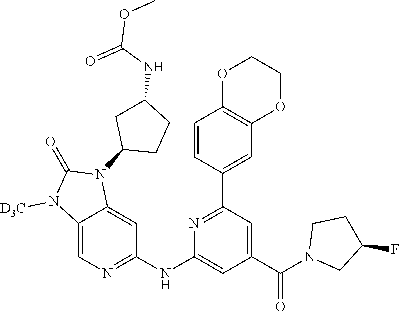 [SMILES: [2H]C([2H])([2H])n1c(=O)n([C@@H]2CC[C@@H](NC(=O)OC)C2)c2cc(Nc3cc(C(=O)N4CC[C@@H](F)C4)cc(-c4ccc5c(c4)OCCO5)n3)ncc21]

The title compound was prepared according to the procedures described in Example 76, with (R)-3-fluoropyrrolidine replacing octahydro-2H-pyrido[1,2-a]pyrazine. LCMS calculated for C₃₂H₃₂D₃FN₇O₆ (M+H)⁺: m/z=635.3; found 635.3. ¹H NMR (400 MHZ, DMSO-d₆) δ 10.50 (s, 1H), 8.23 (s, 1H), 7.66-7.48 (m, 4H), 7.31 (dd, J=16.0, 9.3 Hz, 2H), 6.99 (d, J=8.4 Hz, 1H), 5.38 (dd, J=52.9, 35.4 Hz, 1H), 4.92 (p, J=8.3 Hz, 1H), 4.31 (s, 4H), 4.16 (d, J=7.6 Hz, 1H), 3.84-3.69 (m, 2H), 3.67-3.56 (m, 2H), 3.54 (s, 3H), 2.56 (t, J=5.5 Hz, 1H), 2.35-2.20 (m, 2H), 2.20-1.98 (m, 4H), 1.92 (m, 1H), 1.60-1.53 (m, 1H),

### Example 81. Methyl ((1R,3R)-3-(3-methyl-2-oxo-6-((1-(pyridin-4-yl)-1H-pyrazol-3-yl)amino)-2,3-dihydro-1H-imidazo[4,5-c]pyridin-1-yl)cyclopentyl)carbamate

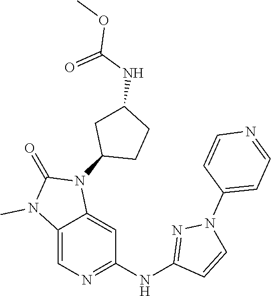 [SMILES: COC(=O)N[C@@H]1CC[C@@H](n2c(=O)n(C)c3cnc(Nc4ccn(-c5ccncc5)n4)cc32)C1]

The title compound was prepared according to the procedures described in Example 53, with 1-(pyridin-4-yl)-1H-pyrazol-3-amine replacing methyl (3-amino-5-(1-methyl-1H-pyrazol-4-yl)phenyl)carbamate in Step 4. LCMS calculated for C₂₂H₂₅N₈O₃ (M+H)⁺: m/z=449.2; found: 449.2. ¹H NMR (600 MHz, DMSO) δ 10.37 (bs, 1H), 8.90-8.79 (m, 3H), 8.16 (d, J=6.4 Hz, 2H), 8.13 (s, 1H), 7.48 (s, 1H), 7.44 (d, J=7.1 Hz, 1H), 6.75 (d, J=2.9 Hz, 1H), 4.99 (p, J=8.9 Hz, 1H), 4.24 (m, 1H), 3.53 (s, 3H), 3.36 (s, 3H), 2.34 (dt, J=13.5, 8.3 Hz, 1H), 2.29-2.22 (m, 1H), 2.16-2.01 (m, 2H), 1.94 (ddd, J=13.9, 9.4, 4.7 Hz, 1H), 1.70-1.64 (m, 1H).

### Example 82. Methyl ((1R,3R)-3-(6-((6-(2,3-dihydrobenzo[b][1,4]dioxin-6-yl)-4-(morpholine-4-carbonyl)pyridin-2-yl)amino)-3-(methyl-d₃)-2-oxo-2,3-dihydro-1H-imidazo[4,5-c]pyridin-1-yl)cyclopentyl)carbamate

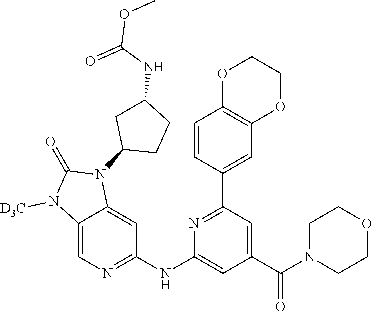 [SMILES: [2H]C([2H])([2H])n1c(=O)n([C@@H]2CC[C@@H](NC(=O)OC)C2)c2cc(Nc3cc(C(=O)N4CCOCC4)cc(-c4ccc5c(c4)OCCO5)n3)ncc21]

The title compound was prepared according to the procedures described in Example 76, with morpholine replacing octahydro-2H-pyrido[1,2-a]pyrazine. LCMS calculated for C₃₂H₃₃D₃N₇O₇ (M+H)⁺: m/z=633.3; found 633.3. ¹H NMR (400 MHZ, DMSO-d₆) δ 10.56 (s, 1H), 8.25 (s, 1H), 7.61 (d, J=2.2 Hz, 1H), 7.58 (dd, J=8.4, 2.2 Hz, 1H), 7.53 (s, 1H), 7.45 (s, 1H), 7.33 (d, J=6.8 Hz, 1H), 7.19 (s, 1H), 6.99 (d, J=8.4 Hz, 1H), 4.92 (p, J=8.4 Hz, 1H), 4.31 (s, 4H), 4.16 (d, J=6.9 Hz, 1H), 3.68 (d, J=12.4 Hz, 4H), 3.59 (s, 2H), 3.54 (s, 3H), 3.38 (s, 2H), 2.29 (m, 1H), 2.15-1.98 (m, 3H), 1.92 (m, 1H), 1.57 (d, J=12.9 Hz, 1H).

### Example 83. Methyl ((1R,3R)-3-(6-((2-((3,3-difluorocyclopentyl)amino)pyrimidin-4-yl)amino)-3-methyl-2-oxo-2,3-dihydro-1H-imidazo[4,5-c]pyridin-1-yl)cyclopentyl)carbamate

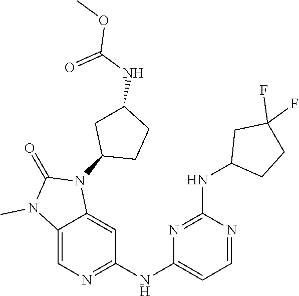 [SMILES: COC(=O)N[C@@H]1CC[C@@H](n2c(=O)n(C)c3cnc(Nc4ccnc(NC5CCC(F)(F)C5)n4)cc32)C1]

The title compound was prepared according to the procedures described in Example 66, with 3,3-difluorocyclopentan-1-amine replacing piperidine in Step 1. LCMS calculated for C₂₃H₂₉F₂N₈O₃ (M+H)⁺: m/z=503.2; found: 503.2.

### Example 84. Methyl ((1R,3R)-3-(6-((2-(3,3-difluoropyrrolidin-1-yl)-6-methylpyrimidin-4-yl)amino)-3-methyl-2-oxo-2,3-dihydro-1H-imidazo[4,5-c]pyridin-1-yl)cyclopentyl)carbamate

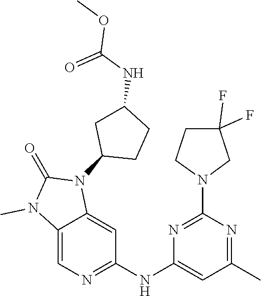

### Step 1. 2-(3,3-Difluoropyrrolidin-1-yl)-6-methylpyrimidin-4-amine

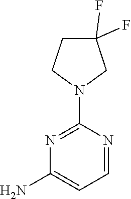 [SMILES: Nc1ccnc(N2CCC(F)(F)C2)n1]

The title compound was prepared according to the procedures described in Example 66, with 3,3-difluoropyrrolidine replacing piperidine and 2-chloro-6-methylpyrimidin-4-amine replacing 2-chloropyrimidin-4-amine in Step 1. LCMS calculated for C₉H₁₃F₂N₄ (M+H)⁺: m/z=215.1; found 215.1.

### Step 2. Methyl ((1R,3R)-3-(6-((2-(3,3-difluoropyrrolidin-1-yl)-6-methylpyrimidin-4-yl)amino)-3-methyl-2-oxo-2,3-dihydro-1H-imidazo[4,5-c]pyridin-1-yl)cyclopentyl)carbamate

The title compound was prepared according to the procedures described in Example 53, with 2-(3,3-difluoropyrrolidin-1-yl)-6-methylpyrimidin-4-amine replacing methyl (3-amino-5-(1-methyl-1H-pyrazol-4-yl)phenyl)carbamate in Step 4. LCMS calculated for C₂₃H₂₉F₂N₈O₃ (M+H)⁺: m/z=503.2; found: 503.2.

### Example 85. Methyl ((1R,3R)-3-(3-methyl-2-oxo-6-((2-((tetrahydro-2H-pyran-4-yl)amino)pyrimidin-4-yl)amino)-2,3-dihydro-1H-imidazo[4,5-c]pyridin-1-yl)cyclopentyl)carbamate

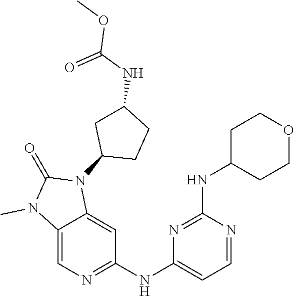 [SMILES: COC(=O)N[C@@H]1CC[C@@H](n2c(=O)n(C)c3cnc(Nc4ccnc(NC5CCOCC5)n4)cc32)C1]

The title compound was prepared according to the procedures described in Example 66, with tetrahydro-2H-pyran-4-amine replacing piperidine in Step 1. LCMS calculated for C₂₃H₃₁N₈O₄ (M+H)⁺: m/z=483.2; found: 483.2.

### Example 86. Methyl ((1R,3R)-3-(3-methyl-2-oxo-6-((6-(piperidine-1-carbonyl)pyridin-2-yl)amino)-2,3-dihydro-1H-imidazo[4,5-c]pyridin-1-yl)cyclopentyl)carbamate

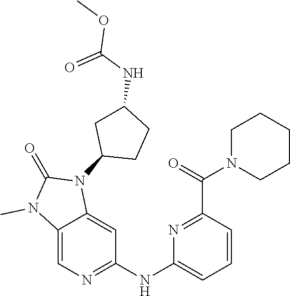 [SMILES: COC(=O)N[C@@H]1CC[C@@H](n2c(=O)n(C)c3cnc(Nc4cccc(C(=O)N5CCCCC5)n4)cc32)C1]

### Step 1. Methyl 6-((1-((1R,3R)-3-((methoxycarbonyl)amino)cyclopentyl)-3-methyl-2-oxo-2,3-dihydro-1H-imidazo[4,5-c]pyridin-6-yl)amino)picolinate

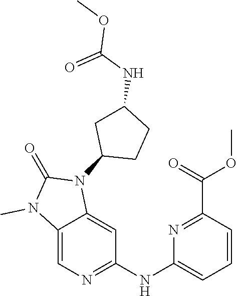

A vial containing methyl ((1R,3R)-3-(6-chloro-3-methyl-2-oxo-2,3-dihydro-1H-imidazo[4,5-c]pyridin-1-yl)cyclopentyl)carbamate (150 mg, 0.462 mmol, Intermediate A), methyl 6-aminopicolinate (141 mg, 0.924 mmol), cesium carbonate (301 mg, 0.924 mmol), 4,5-bis(diphenylphosphino)-9,9-dimethylxanthene (80 mg, 0.139 mmol) and tris(dibenzylideneacetone)dipalladium(0) (63 mg, 0.069 mmol) was evacuated and backfilled with nitrogen three times, followed by the addition of dioxane (4.5 ml). The vial was sealed and heated to 100° C. for 16 h. After cooling to room temperature, the mixture was filtered through a SiliaPrep SPE thiol cartridge (SPE-R51030B-06P) and washed with CH₂Cl₂. The filtrate was concentrated in vacuo and was washed with aqueous saturated sodium bicarbonate solution. The organic phase was washed with brine, dried over MgSO₄ and concentrated in vacuo. The obtained crude product was purified by Biotage Isolera to give the desired product as white solid. LCMS calculated for C₂₁H₂₅N₆O₅ (M+H)⁺: m/z=441.2; found 441.2.

### Step 2. Methyl ((1R,3R)-3-(3-methyl-2-oxo-6-((6-(piperidine-1-carbonyl)pyridin-2-yl)amino)-2,3-dihydro-1H-imidazo[4,5-c]pyridin-1-yl)cyclopentyl) carbamate

To a vial containing methyl 6-((1-((1R,3R)-3-((methoxycarbonyl)amino)cyclopentyl)-3-methyl-2-oxo-2,3-dihydro-1H-imidazo[4,5-c]pyridin-6-yl)amino)picolinate (10 mg, 23 μmol) and piperidine (4 mg, 45 μmol) in acetonitrile (0.3 mL) was added magnesium bromide (6 mg, 34 μmol). The reaction mixture was heated to 50° C. for 2 h. After cooling to r.t., the mixture was diluted with acetonitrile, filtered and purified with prep-LCMS (XBridge C18 column, eluting with a gradient of acetonitrile/water containing 0.1% TFA, at flow rate of 60 mL/min). LCMS calculated for C₂₅H₃₂N₇O₄ (M+H)⁺: m/z=494.2; found: 494.2. ¹H NMR (600 MHz, DMSO) δ 10.72 (bs, 1H), 8.22 (s, 1H), 7.89 (s, 1H), 7.45-7.28 (m, 3H), 7.09 (s, 1H), 4.94 (p, J=8.7 Hz, 1H), 4.21 (m, 1H), 3.64 (m, 2H), 3.55 (s, 3H), 3.36 (s, 3H), 3.30 (t, J=5.5 Hz, 2H), 2.29-2.17 (m, 2H), 2.14-1.98 (m, 2H), 1.91 (ddd, J=13.9, 9.4, 4.7 Hz, 1H), 1.69-1.56 (m, 5H), 1.44 (m, 2H).

### Example 87. Methyl ((1R,3R)-3-(6-((6-(cyclohexylcarbamoyl)pyridin-2-yl)amino)-3-methyl-2-oxo-2,3-dihydro-1H-imidazo[4,5-c]pyridin-1-yl)cyclopentyl)carbamate

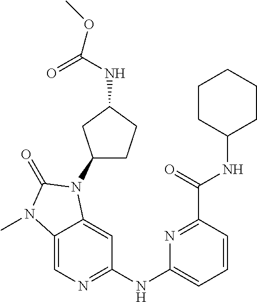 [SMILES: COC(=O)N[C@@H]1CC[C@@H](n2c(=O)n(C)c3cnc(Nc4cccc(C(=O)NC5CCCCC5)n4)cc32)C1]

The title compound was prepared according to the procedures described in Example 86, with cyclohexanamine replacing piperidine in Step 2. LCMS calculated for C₂₆H₃₄N₇O₄ (M+H)⁺: m/z=508.3; found: 508.2.

### Example 88. Methyl ((1R,3R)-3-(6-((1-(2-methoxyethyl)-3-(1-(trifluoromethyl)-1H-pyrazol-4-yl)-1H-pyrrolo[3,2-b]pyridin-5-yl)amino)-3-(methyl-d₃)-2-oxo-2,3-dihydro-1H-imidazo[4,5-c]pyridin-1-yl)cyclopentyl)carbamate

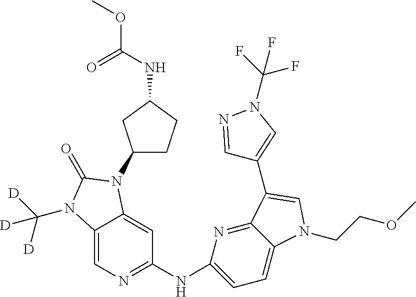 [SMILES: [2H]C([2H])([2H])n1c(=O)n([C@@H]2CC[C@@H](NC(=O)OC)C2)c2cc(Nc3ccc4c(n3)c(-c3cnn(C(F)(F)F)c3)cn4CCOC)ncc21]

### Step 1. (E)-N,N-Dimethyl-N′-(1H-pyrrolo[3,2-b]pyridin-5-yl)formimidamide

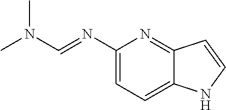

To a flask containing 1H-pyrrolo[3,2-b]pyridin-5-amine (1.78 g, 13.37 mmol) in methanol (10 mL) was added 1,1-dimethoxy-N,N-dimethylmethanamine (2.31 mL, 17.38 mmol). The reaction was heated to 80° C. for 2 h and cooled down and concentrated to dryness and it was used for next step without further purification. LCMS calculated for C₁₀H₁₃N₄ (M+H)⁺: m/z=189.1; found 189.1.

### Step 2. (E)-N′-(3-Iodo-1H-pyrrolo[3,2-b]pyridin-5-yl)-N,N-dimethylformimidamide

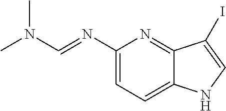

To a flask containing (E)-N,N-dimethyl-N′-(1H-pyrrolo[3,2-b]pyridin-5-yl)formimidamide (2.50 g, 13.28 mmol) in DMF (15 mL) was added NIS (3.88 g, 17.27 mmol) at 0° C. The reaction was stirred at r.t. for 2 h and then quenched with Na₂S₂O₃ aqueous solution. The mixture was extracted with CH₂Cl₂ and the organic layer was washed with water and brine, dried over Na₂SO₄, concentrated and purified by Biotage Isolera to give the product as a brown solid. LCMS calculated for C₁₀H₁₂IN₄ (M+H)⁺: m/z=315.0; found: 315.1.

### Step 3. (E)-N′-(3-Iodo-1-(2-methoxyethyl)-1H-pyrrolo[3,2-b]pyridin-5-yl)-N,N-dimethylformimidamide

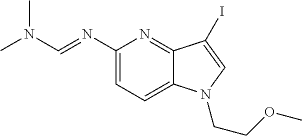

(E)-N′-(3-Iodo-1H-pyrrolo[3,2-b]pyridin-5-yl)-N,N-dimethylformimidamide (0.30 g, 0.955 mmol) was dissolved in DMF (3 mL) and cooled to 0° C., then NaH (0.046 g, 1.146 mmol, 60% w/w) was added, and the reaction was stirred at 0° C. for 30 min. Then 1-bromo-2-methoxyethane (0.159 g, 1.146 mmol) in THF (2 mL) was added. The reaction was stirred at r.t. for 2 h and then quenched with water. The mixture was extracted with CH₂Cl₂ and the organic layer was washed with water and brine, then dried over Na₂SO₄. It was concentrated in vacuo and purified by Biotage Isolera to give the product as a brown solid. LCMS calculated for C₁₃H₁₈IN₄O (M+H)⁺: m/z=373.0; found: 373.2.

### Step 4. (E)-N′-(1-(2-Methoxyethyl)-3-(1-(trifluoromethyl)-1H-pyrazol-4-yl)-1H-pyrrolo[3,2-b]pyridin-5-yl)-N,N-dimethylformimidamide

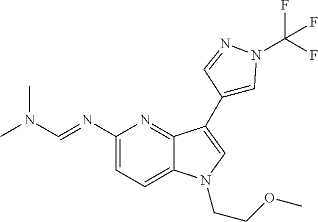 [SMILES: COCCn1cc(-c2cnn(C(F)(F)F)c2)c2nc(/N=C/N(C)C)ccc21]

(E)-N-(3-Iodo-1-(2-methoxyethyl)-1H-pyrrolo[3,2-b]pyridin-5-yl)-N,N-dimethylformimidamide (0.150 g, 0.403 mmol), 4-(4,4,5,5-tetramethyl-1,3,2-dioxaborolan-2-yl)-1-(trifluoromethyl)-1H-pyrazole (0.116 g, 0.443 mmol), dppf palladium(II) (0.066 g, 0.081 mmol) and potassium phosphate, dibasic (0.211 g, 1.209 mmol) were mixed in vial and the vial was purged with N2. Dioxane (5 mL) and water (1.2 mL) were added and the reaction mixture was stirred at 70° C. for 1 h. The mixture was quenched with water and diluted with ethyl acetate. The organic phase was separated, washed with water and brine, dried over sodium sulfate and concentrated. The residue was purified by Biotage Isolera to give the product as a brown solid. LCMS calculated for C₁₇H₂₀F₃N₆O (M+H)⁺: m/z=381.2; found: 381.2.

### Step 5. 1-(2-Methoxyethyl)-3-(1-(trifluoromethyl)-1H-pyrazol-4-yl)-1H-pyrrolo[3,2-b]pyridin-5-amine

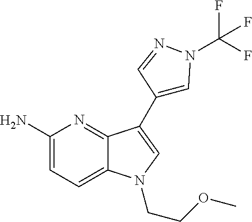 [SMILES: COCCn1cc(-c2cnn(C(F)(F)F)c2)c2nc(N)ccc21]

(E)-N-(1-(2-methoxyethyl)-3-(1-(trifluoromethyl)-1H-pyrazol-4-yl)-1H-pyrrolo[3,2-b]pyridin-5-yl)-N,N-dimethylformimidamide (0.12 g, 0.315 mmol) was mixed with MeOH (5 mL) and water (1.2 mL) in a vial and KOH (0.077 g, 1.370 mmol) was added. The reaction mixture was stirred at 75° C. for 3 h. The mixture was then diluted with ethyl acetate and washed with water and brine, dried over sodium sulfate and concentrated. The residue was purified by Biotage Isolera to give the product as a brown solid. LCMS calculated for C₁₄H₁₅F₃N₅O (M+H)⁺: m/z=326.1; found: 326.2.

### Step 6. Methyl ((1R,3R)-3-(6-((1-(2-methoxyethyl)-3-(1-(trifluoromethyl)-1H-pyrazol-4-yl)-1H-pyrrolo[3,2-b]pyridin-5-yl)amino)-3-(methyl-d3)-2-oxo-2,3-dihydro-1H-imidazo[4,5-c]pyridin-1-yl)cyclopentyl)carbamate

The vial containing methyl ((1R,3R)-3-(6-chloro-3-(methyl-d₃)-2-oxo-2,3-dihydro-1H-imidazo[4,5-c]pyridin-1-yl)cyclopentyl)carbamate (50 mg, 0.153 mmol), 1-(2-methoxyethyl)-3-(1-(trifluoromethyl)-1H-pyrazol-4-yl)-1H-pyrrolo[3,2-b]pyridin-5-amine (50 mg, 0.153 mmol), tris(dibenzylideneacetone)dipalladium(0) (14 mg, 0.015 mmol), 4,5-bis(diphenylphosphino)-9,9-dimethylxanthene (18 mg, 0.031 mmol) and Cs₂CO₃ (149 mg, 0.458 mmol) was evacuated and backfilled with nitrogen three times. Then 1,4-dioxane (4.0 mL) was added. The reaction mixture was heated to 110° C. for 2h. The mixture was then cooled to r.t., diluted with MeCN and purified by prep-LCMS (XBridge C18 column, eluting with a gradient of acetonitrile/water containing 0.1% TFA, at flow rate of 60 mL/min). LCMS calculated for C₂₈H₂₈D₃F₃N₉O₄ (M+H)⁺: m/z=617.3; found: 617.3. ¹H NMR (400 MHZ, DMSO-d₆) δ 11.02 (s, 1H), 8.84 (s, 1H), 8.52 (s, 1H), 8.20 (d, J=8.9 Hz, 1H), 8.12-8.06 (m, 2H), 7.35 (d, J=7.2 Hz, 1H), 7.17 (s, 1H), 7.11 (d, J=8.7 Hz, 1H), 4.94 (p, J=8.4 Hz, 1H), 4.44 (t, J=5.0 Hz, 2H), 4.24-4.16 (m, 1H), 3.71 (t, J=5.0 Hz, 2H), 3.55 (s, 3H), 3.24 (s, 3H), 2.28 (m, 1H), 2.21-2.09 (m, 2H), 2.08-1.87 (m, 1H), 1.67-1.57 (m, 1H).

### Example 89. Methyl ((1R,3R)-3-(6-((3-(1-(difluoromethyl)-1H-pyrazol-4-yl)-1-methyl-2-oxo-7-(4-(trifluoromethyl)pyridin-3-yl)-2,3-dihydro-1H-imidazo[4,5-b]pyridin-5-yl)amino)-3-(methyl-d₃)-2-oxo-2,3-dihydro-1H-imidazo[4,5-c]pyridin-1-yl)cyclopentyl)carbamate

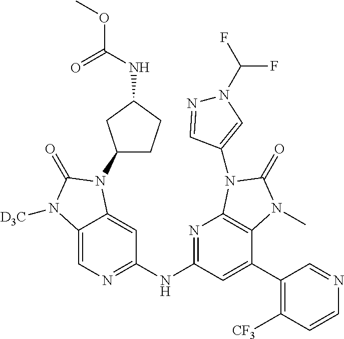 [SMILES: [2H]C([2H])([2H])n1c(=O)n([C@@H]2CC[C@@H](NC(=O)OC)C2)c2cc(Nc3cc(-c4cnccc4C(F)(F)F)c4c(n3)n(-c3cnn(C(F)F)c3)c(=O)n4C)ncc21]

### Step 1. tert-Butyl (2,6-dichloro-3-nitropyridin-4-yl)carbamate

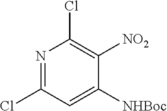 [SMILES: CC(C)(C)OC(=O)Nc1cc(Cl)nc(Cl)c1[N+](=O)[O-]]

This compound was prepared according to the procedures described in WO 2023/011359. LCMS calculated for C₁₀H₁₂C₁₂N304 (M+H)⁺: m/z=308.0; found: 308.1.

### Step 2. tert-Butyl (6-chloro-2-((1-(difluoromethyl)-1H-pyrazol-4-yl)amino)-3-nitropyridin-4-yl)carbamate

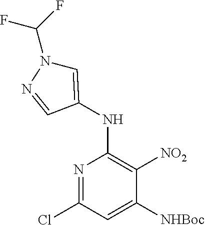 [SMILES: CC(C)(C)OC(=O)Nc1cc(Cl)nc(Nc2cnn(C(F)F)c2)c1[N+](=O)[O-]]

To a solution of tert-butyl (2,6-dichloro-3-nitropyridin-4-yl)carbamate (1.50 g, 4.87 mmol) and 1-(difluoromethyl)-1H-pyrazol-4-amine (0.972 g, 7.30 mmol) in ethanol (20 mL) was added sodium bicarbonate (0.695 g, 8.28 mmol). After stirring at r.t. for 62 h, the reaction mixture was diluted with water (40 mL) and extracted into 150 mL of ethyl acetate. The ethyl acetate layer was washed with brine, dried over anhydrous sodium sulfate, filtered, and concentrated in vacuo to afford orange solid. The crude material was purified by Biotage Isolera to give the product. LCMS calculated for C₁₄H₁₆ClF₂N₆O₄ (M+H)⁺: m/z=405.1; found: 405.1.

### Step 3. tert-Butyl (3-amino-6-chloro-2-((1-(difluoromethyl)-1H-pyrazol-4-yl)amino)pyridin-4-yl)carbamate

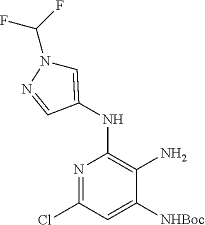 [SMILES: CC(C)(C)OC(=O)Nc1cc(Cl)nc(Nc2cnn(C(F)F)c2)c1N]

To a stirred suspension of tert-butyl (6-chloro-2-((1-(difluoromethyl)-1H-pyrazol-4-yl)amino)-3-nitropyridin-4-yl)carbamate (0.4 g, 0.988 mmol) in solvents mixture (9.8 mL, THF/MeOH/water, 3/2/1 v/v/v) were added ammonium chloride (0.16 g, 2.96 mmol) and iron powder (0.5 g, 9.88 mmol). After stirring at 70° C. for 1 h, a new solvents mixture (4.9 mL, THF/MeOH/water, 3/2/1 v/v/v) was added to the reaction mixture followed by ammonium chloride (0.079 g, 1.48 mmol) and iron powder (0.28 g, 4.94 mmol). After stirring at 70° C. for another 1 h, the reaction mixture was diluted with ethyl acetate and was filtered through a 0.45 micron filter cartridge. The filtrate was washed with saturated sodium bicarbonate solution, brine, dried over anhydrous sodium sulfate, filtered, and concentrated in vacuo give the desired product as a purple foam. The resulting crude material was used in next step without further purification. LCMS calculated for C₁₄H₁₈ClF₂N₆O₂ (M+H)⁺: m/z=375.1; found: 375.1.

### Step 4. tert-Butyl (5-chloro-3-(1-(difluoromethyl)-1H-pyrazol-4-yl)-2-oxo-2,3-dihydro-1H-imidazo[4,5-b]pyridin-7-yl)carbamate

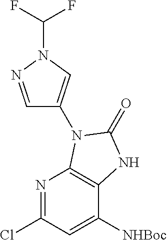 [SMILES: CC(C)(C)OC(=O)Nc1cc(Cl)nc2c1[nH]c(=O)n2-c1cnn(C(F)F)c1]

To a solution of tert-butyl (3-amino-6-chloro-2-((1-(difluoromethyl)-1H-pyrazol-4-yl)amino)pyridin-4-yl)carbamate (0.6 g, 1.60 mmol) in acetonitrile (13 mL) was added carbonyldiimidazole (1 g, 6.40 mmol). After stirring at 70° C. for 1 h, water (10 mL) was added dropwise to the reaction mixture with gas evolution and stirred for 30 min. The reaction mixture was diluted with water (40 mL) and extracted with ethyl acetate (50 mL). The organic layer was washed with 20 mL of brine, dried over anhydrous sodium sulfate, filtered, and concentrated to dryness. The crude solid was suspended in dichloromethane and filtered to yield desired product. The filtrate was concentrated and further purified by flash column chromatography to give the second portion of desired product. This product was combined with the previously isolated solid to give the final product as a pink solid. LCMS calculated for C₁₅H₁₆ClF₂N₆O₃ (M+H)⁺: m/z=401.1; found: 401.1.

### Step 5. 7-Amino-5-chloro-3-(1-(difluoromethyl)-1H-pyrazol-4-yl)-1,3-dihydro-2H-imidazo[4,5-b]pyridin-2-one

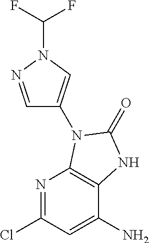 [SMILES: Nc1cc(Cl)nc2c1[nH]c(=O)n2-c1cnn(C(F)F)c1]

To a solution of tert-butyl (5-chloro-3-(1-(difluoromethyl)-1H-pyrazol-4-yl)-2-oxo-2,3-dihydro-1H-imidazo[4,5-b]pyridin-7-yl)carbamate (0.06 g, 0.150 mmol) in dichloromethane (0.5 mL) was added TFA dropwise (0.5 mL). After stirring at r.t. for 2 h, the mixture was concentrated in vacuo. The crude product was diluted with ethyl acetate (30 mL) and neutralized with saturated sodium bicarbonate solution (20 mL). The ethyl acetate extract was washed with brine, dried over anhydrous sodium sulfate, filtered, and concentrated in vacuo to desired product as a pink solid. The resulting crude material was used in next step without further purification. LCMS calculated for C₁₀H₈ClF₂N₆O (M+H)⁺: m/z=301.0; found: 301.1.

### Step 6. 7-Amino-5-chloro-3-(1-(difluoromethyl)-1H-pyrazol-4-yl)-1-methyl-1,3-dihydro-2H-imidazo[4,5-b]pyridin-2-one

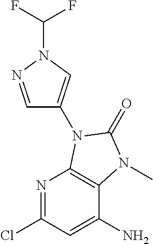 [SMILES: Cn1c(=O)n(-c2cnn(C(F)F)c2)c2nc(Cl)cc(N)c21]

To an ice cooled solution of 7-amino-5-chloro-3-(1-(difluoromethyl)-1H-pyrazol-4-yl)-1,3-dihydro-2H-imidazo[4,5-b]pyridin-2-one (0.24 g, 0.788 mmol) in DMF (4 mL) was added cesium carbonate (0.64 g, 1.97 mmol) followed by dropwise addition of methyl iodide (1.2 mL, 1.18 mmol, 1.0 M in DMF). The cooling bath was removed and the mixture was stirred for 30 min at r.t. The reaction mixture was diluted with water (25 mL) and was extracted into ethyl acetate (50 mL). The EtOAc extract was washed with brine, dried over anhydrous sodium sulfate, filtered, and concentrated in-vacuo to tan solid. The crude solid was suspended in dichloromethane and filtered to yield desired product. The filtrate was further purified by flash column chromatography to give the second portion of desired product. This product was combined with the previously isolated solid to give the final product as a tan solid. LCMS calculated for C₁₁H₁₀ClF₂N₆O (M+H)⁺: m/z=315.1; found: 315.1.

### Step 7. 7-Bromo-5-chloro-3-(1-(difluoromethyl)-1H-pyrazol-4-yl)-1-methyl-1,3-dihydro-2H-imidazo[4,5-b]pyridin-2-one

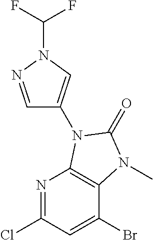 [SMILES: Cn1c(=O)n(-c2cnn(C(F)F)c2)c2nc(Cl)cc(Br)c21]

To a mixture of copper(II) bromide (0.2 g, 0.872 mmol) and 7-amino-5-chloro-3-(1-(difluoromethyl)-1H-pyrazol-4-yl)-1-methyl-1,3-dihydro-2H-imidazo[4,5-b]pyridin-2-one (0.21 g, 0.671 mmol) in acetonitrile (3.4 mL) and THF (3.4 mL) at 0° C. was added tert-butyl nitrite (0.133 mL, 1.01 mmol). After the addition, the reaction mixture was stirred for 1 h at r.t. followed by another 1 h at 50° C. The reaction mixture was cooled to r.t. and diluted with sat. sodium bicarbonate solution (20 mL) and ethyl acetate (50 mL). The mixture was stirred for 5 min and the solids were removed by filtration through a 0.45 micron filter cartridge. The ethyl acetate layer was washed with brine, dried over anhydrous sodium sulfate, filtered, and concentrated to a tan residue. The material was purified by flash column chromatography to give approximately a 1:1 mixture of desired 7-bromo-5-chloro-3-(1-(difluoromethyl)-1H-pyrazol-4-yl)-1-methyl-1,3-dihydro-2H-imidazo[4,5-b]pyridin-2-one and side product 6,7-dibromo-5-chloro-3-(1-(difluoromethyl)-1H-pyrazol-4-yl)-1-methyl-1,3-dihydro-2H-imidazo[4,5-b]pyridin-2-one as a white solid. LCMS calculated for C₁₁H₈BrClF₂N₅O (M+H)⁺: m/z=378.0, 380.0; found: 378.0, 380.0.

### Step 8. 5-Chloro-3-(1-(difluoromethyl)-1H-pyrazol-4-yl)-1-methyl-7-(4-(trifluoromethyl)pyridin-3-yl)-1,3-dihydro-2H-imidazo[4,5-b]pyridin-2-one

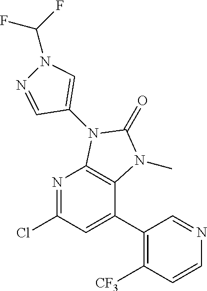 [SMILES: Cn1c(=O)n(-c2cnn(C(F)F)c2)c2nc(Cl)cc(-c3cnccc3C(F)(F)F)c21]

A reaction vial containing a mixture of 7-bromo-5-chloro-3-(1-(difluoromethyl)-1H-pyrazol-4-yl)-1-methyl-1,3-dihydro-2H-imidazo[4,5-b]pyridin-2-one and 6,7-dibromo-5-chloro-3-(1-(difluoromethyl)-1H-pyrazol-4-yl)-1-methyl-1,3-dihydro-2H-imidazo[4,5-b]pyridin-2-one (40.2 mg), 3-(4,4,5,5-tetramethyl-1,3,2-dioxaborolan-2-yl)-4-(trifluoromethyl)pyridine (145 mg, 0.531 mmol), cesium carbonate (104 mg, 0.318 mmol), and tetrakis-(triphenylphosphine)palladium(0) (12 mg, 10.6 μmol) was evacuated and backfilled with nitrogen, followed by the addition of dioxane (3 mL) and water (0.2 mL). After stirring at 100° C. for 2.5 h, the reaction mixture was cooled to r.t and was diluted with ethyl acetate. The organic layer was subsequently washed with water and brine, dried over sodium sulfate, filtered, and concentrated in vacuo. The crude material was purified by Biotage Isolera using ethyl acetate (containing 10% methanol) in hexanes to give the product as a white residue. LCMS calculated for C₁₂H₁₁ClF₅N₆O (M+H)⁺: m/z=445.1; found: 445.1.

### Step 9. Methyl ((1R,3R)-3-(6-((3-(1-(difluoromethyl)-1H-pyrazol-4-yl)-1-methyl-2-oxo-7-(4-(trifluoromethyl)pyridin-3-yl)-2,3-dihydro-1H-imidazo[4,5-b]pyridin-5-yl)amino)-3-(methyl-d₃)-2-oxo-2,3-dihydro-1H-imidazo[4,5-c]pyridin-1-yl)cyclopentyl)carbamate

A reaction vial containing methyl ((1R,3R)-3-(6-amino-3-(methyl-d₃)-2-oxo-2,3-dihydro-1H-imidazo[4,5-c]pyridin-1-yl)cyclopentyl)carbamate (11 mg, 0.035 mmol, Example 52, Step 4), 5-chloro-3-(1-(difluoromethyl)-1H-pyrazol-4-yl)-1-methyl-7-(4-(trifluoromethyl)pyridin-3-yl)-1,3-dihydro-2H-imidazo[4,5-b]pyridin-2-one (12 mg, 27 μmol), Xantphos (6 mg, 10.8 μmol), tris(dibenzylideneacetone)dipalladium (0) (5 mg, 5.4 μmol), and cesium carbonate (26 mg, 0.081 mmol) was evacuated and backfilled with nitrogen 3 times, followed by the addition of dioxane (0.45 mL). The vial was sealed and heated to 100° C. for 22 h. The reaction mixture was diluted with ethyl acetate, filtered through a 0.45 micron filter cartridge, and concentrated in vacuo. The mixture was then diluted with acetonitrile and purified with prep-LCMS (XBridge C18 column, eluting with a gradient of acetonitrile/water containing 0.1% TFA, at flow rate of 60 mL/min). LCMS calculated for C₃₁H₂₆D₃F₅N₁₁O₄(M+H)⁺: m/z=717.2; found: 717.3. ¹H NMR (500 MHZ, DMSO-d₆) δ: 10.23 (br s, 1H), 9.07 (d, J=5.2 Hz, 1H), 8.95 (s, 1H), 8.83 (s, 1H), 8.54 (s, 1H), 8.07 (s, 1H), 8.02 (d, J=5.3 Hz, 1H), 7.93 (t, J=58.2 Hz, 1H), 7.32-7.23 (m, 3H), 4.93-4.82 (m, 1H), 4.18 (q, J=6.9 Hz, 1H), 3.53 (s, 3H), 2.80 (s, 3H), 2.29-2.20 (m, 1H), 2.17-2.04 (m, 2H), 2.03-1.92 (m, 1H), 1.92-1.82 (m, 1H), 1.62-1.52 (m, 1H).

### Example 90. N-(6-(3-(Methyl-d₃)-6-((4-(morpholine-4-carbonyl)-8-(trifluoromethyl)quinolin-2-yl)amino)-2-oxo-2,3-dihydro-1H-imidazo[4,5-c]pyridin-1-yl)spiro[3,3]heptan-2-yl)cyclopropanecarboxamide

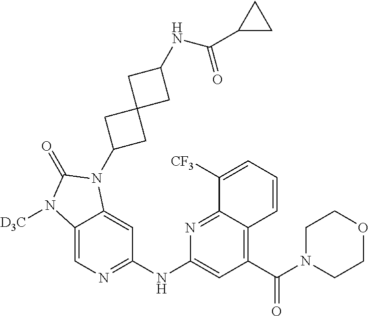 [SMILES: [2H]C([2H])([2H])n1c(=O)n(C2CC3(CC(NC(=O)C4CC4)C3)C2)c2cc(Nc3cc(C(=O)N4CCOCC4)c4cccc(C(F)(F)F)c4n3)ncc21]

The title compound was prepared according to the procedures described for Intermediate A and Example 52, using tert-butyl (6-aminospiro[3.3]heptan-2-yl)carbamate as starting material. LCMS calculated for C₃₃H₃₂D₃F₃N₇O₄ (M+H)⁺: m/z=653.3; found: 653.4

### Example 91. N-((1s,4s)-1-Methyl-4-(3-(methyl-d₃)-6-((2-methyl-3-(1-(trifluoromethyl)-1H-pyrazol-4-yl)-2H-pyrazolo[4,3-b]pyridin-5-yl)amino)-2-oxo-2,3-dihydro-1H-imidazo[4,5-c]pyridin-1-yl)cyclohexyl)cyclopropanecarboxamide

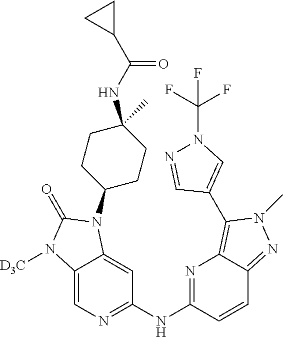 [SMILES: [2H]C([2H])([2H])n1c(=O)n([C@H]2CC[C@@](C)(NC(=O)C3CC3)CC2)c2cc(Nc3ccc4nn(C)c(-c5cnn(C(F)(F)F)c5)c4n3)ncc21]

### Step 1. tert-Butyl ((1s,4s)-4-(6-chloro-3-(methyl-d₃)-2-oxo-2,3-dihydro-1H-imidazo[4,5-c]pyridin-1-yl)-1-methylcyclohexyl)carbamate

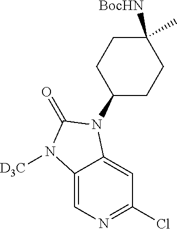 [SMILES: [2H]C([2H])([2H])n1c(=O)n([C@H]2CC[C@@](C)(NC(=O)OC(C)(C)C)CC2)c2cc(Cl)ncc21]

This compound was prepared according to the procedures described in Intermediate A, with tert-butyl ((1s,4s)-4-amino-1-methylcyclohexyl)carbamate replacing tert-butyl ((1R,3R)-3-aminocyclobutyl)carbamate in Step 1. LCMS calculated for C₁₉H₂₅D₃ClN₄O₃ (M+H)⁺: m/z=398.2; found: 398.3.

### Step 2. N-((1s,4s)-4-(6-Chloro-3-(methyl-d₃)-2-oxo-2,3-dihydro-1H-imidazo[4,5-c]pyridin-1-yl)-1-methylcyclohexyl)cyclopropanecarboxamide

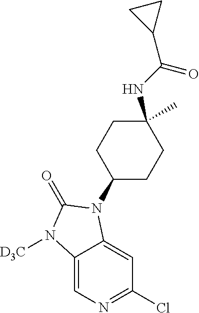 [SMILES: [2H]C([2H])([2H])n1c(=O)n([C@H]2CC[C@@](C)(NC(=O)C3CC3)CC2)c2cc(Cl)ncc21]

4 N HCl (10 mL) was added to a solution of tert-butyl ((1s,4s)-4-(6-chloro-3-(methyl-d₃)-2-oxo-2,3-dihydro-1H-imidazo[4,5-c]pyridin-1-yl)-1-methylcyclohexyl)carbamate (3.18 g, 8.0 mmol) in CH₂Cl₂ (5.0 mL)/MeOH (5.0 mL) and then stirred for 30 min. After the solvent was evaporated in vacuo the residue was dissolved in CH₂Cl₂ (30 mL). Then cyclopropanecarboxylic acid (2.1 g, 24.00 mmol) and triethylamine (5.58 mL, 40.0 mmol) were added at 0° C. Then propane phosphonic acid anhydride (50% in EtOAc) (7.64 g, 24.00 mmol) was added and the reaction was stirred at r.t. for 2 h. The mixture was diluted with CH₂Cl₂, washed with saturated NaHCO₃. The organic phase was concentrated and the product was purified on silica gel (40 g, 0-10% MeOH in CH₂Cl₂). LC-MS calculated for C₁₈H₂₁D₃ClN₄O₂ (M+H)⁺: m/z=366.2; found 366.2.

### Step 3. N-((1s,4s)-4-(6-Amino-3-(methyl-d₃)-2-oxo-2,3-dihydro-1H-imidazo[4,5-c]pyridin-1-yl)-1-methylcyclohexyl)cyclopropanecarboxamide

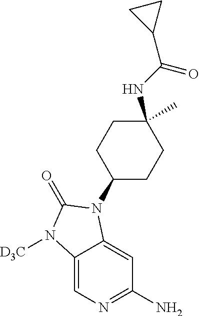 [SMILES: [2H]C([2H])([2H])n1c(=O)n([C@H]2CC[C@@](C)(NC(=O)C3CC3)CC2)c2cc(N)ncc21]

A mixture of N-((1s,4s)-4-(6-chloro-3-(methyl-d₃)-2-oxo-2,3-dihydro-1H-imidazo[4,5-c]pyridin-1-yl)-1-methylcyclohexyl)cyclopropanecarboxamide (0.366 g, 1.0 mmol), tert-butyl carbamate (0.586 g, 5.00 mmol), K₂CO₃ (0.415 g, 3.00 mmol) and BrettPhos Pd G3 (0.091 g, 0.10 mmol) in dioxane (6.0 mL) was evacuated and backfilled with nitrogen (this process was repeated a total of three times) and then the reaction was stirred at 100° C. for 2 h. The mixture was diluted with CH₂Cl₂ and filtered. The organic phase was concentrated and then the obtained intermediate was purified on silica gel (20 g, 0-10% MeOH in CH₂Cl₂). The Boc protecting group was removed in HCl (in dioxane). The reaction mixture was concentrated to remove most of the solvent and neutralized with saturated NaHCO₃, and the product was filtered, washed with water and dried. LC-MS calculated for C₁₈H₂₃D₃N₅O₂ (M+H)⁺: m/z=347.2; found 347.3.

### Step 4. 5-Chloro-3-iodo-2-methyl-2H-pyrazolo[4,3-b]pyridine

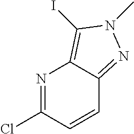

Sodium hydride (0.060 g, 1.5 mmol, 60% w/w) was added to a solution of 5-chloro-3-iodo-1H-pyrazolo[4,3-b]pyridine (0.279 g, 1.0 mmol) in DMF (3.0 mL) at 0° C. and stirred for 10 min, at this time iodomethane (0.426 g, 3.00 mmol) was added to the reaction mixture and then stirred at this temperature for 1 h. Water (3.0 mL) was slowly added to the reaction mixture and the formed precipitate was filtered, washed with water, dried to provide the desired product as a white solid as a mixture of the title compound and another regioisomer 5-chloro-3-iodo-1-methyl-1H-pyrazolo[4,3-b]pyridine. LC-MS calculated for C₇H₆ClIN₃ (M+H)⁺: m/z=293.9; found 293.9.

### Step 5. 5-Chloro-2-methyl-3-(1-(trifluoromethyl)-1H-pyrazol-4-yl)-2H-pyrazolo[4,3-b]pyridine

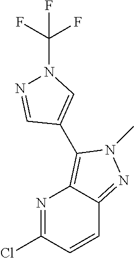 [SMILES: Cn1nc2ccc(Cl)nc2c1-c1cnn(C(F)(F)F)c1]

A mixture of 5-chloro-3-iodo-1-methyl-1H-pyrazolo[4,3-b]pyridine and 5-chloro-3-iodo-2-methyl-2H-pyrazolo[4,3-b]pyridine (0.219 g, 0.75 mmol), 4-(4,4,5,5-tetramethyl-1,3,2-dioxaborolan-2-yl)-1-(trifluoromethyl)-1H-pyrazole (0.131 g, 0.500 mmol), K₃PO₄ (0.212 g, 1.0 mmol) and dichlorobis(triphenylphosphine)-palladium(II) (0.018 g, 0.025 mmol) in dioxane (3.0 mL)/water (0.8 mL) was evacuated and backfilled with nitrogen (this process was repeated a total of three times), then the reaction was stirred at 60° C. for 1 h. The mixture was diluted with ethyl acetate and washed with water, the organic phase was dried and concentrated. The product was purified on silica gel (0-80% EtOAc in Hexane). LC-MS calculated for C₁₁H₈ClF₃N₅ (M+H)⁺: m/z=302.1; found 302.1.

### Step 6. N-((1s,4s)-1-Methyl-4-(3-(methyl-d₃)-6-((2-methyl-3-(1-(trifluoromethyl)-1H-pyrazol-4-yl)-2H-pyrazolo[4,3-b]pyridin-5-yl)amino)-2-oxo-2,3-dihydro-1H-imidazo[4,5-c]pyridin-1-yl)cyclohexyl)cyclopropanecarboxamide

A mixture of N-((1s,4s)-4-(6-amino-3-(methyl-d₃)-2-oxo-2,3-dihydro-1H-imidazo[4,5-c]pyridin-1-yl)-1-methylcyclohexyl)cyclopropanecarboxamide (5 mg, 0.015 mmol), 5-chloro-2-methyl-3-(1-(trifluoromethyl)-1H-pyrazol-4-yl)-2H-pyrazolo[4,3-b]pyridine (5 mg, 0.017 mmol), sodium tert-butoxide (6 mg, 0.060 mmol) and/BuBrettPhos Pd G3 (3.7 mg, 4.33 μmol) in THF (1.0 mL) was evacuated and backfilled with nitrogen (this process was repeated a total of three times), and then the reaction was stirred at r.t. for 1.5 h. The pH of the reaction mixture was adjusted to 1 by adding IN HCl, then the reaction was diluted with MeCN and purified by prep-LCMS as the first peak of two regioisomers (XBridge C18 column, eluting with a gradient of acetonitrile/water containing 0.1% TFA, at flow rate of 60 mL/min). LCMS calculated for C₂₉H₂₉D₃F₃N₁₀O₂ (M+H)⁺: m/z=612.3; found: 612.2. ¹H NMR (600 MHz, DMSO) δ 11.09 (s, 1H), 9.11 (s, 1H), 8.75 (s, 1H), 8.18-8.10 (m, 2H), 7.77 (s, 1H), 7.52 (s, 1H), 7.17 (d, J=9.2 Hz, 1H), 4.29 (s, 3H), 4.09 (m, 1H), 2.43 (m, 2H), 2.38-2.32 (m, 2H), 1.68-1.53 (m, 3H), 1.42 (dd, J=13.7, 3.7 Hz, 2H), 1.27 (s, 3H), 0.65-0.47 (m, 4H).

### Example 92. N-((1S,3R)-3-(6-((6-(5-Chlorothiazol-2-yl)-4-(2-hydroxypropan-2-yl)pyridin-2-yl)amino)-3-(methyl-d₃)-2-oxo-2,3-dihydro-1H-imidazo[4,5-c]pyridin-1-yl)-1-methylcyclopentyl)cyclopropanecarboxamide

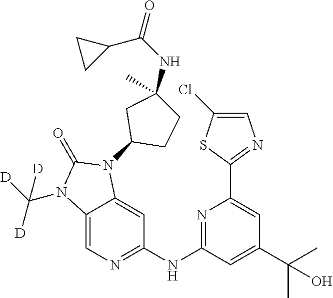 [SMILES: [2H]C([2H])([2H])n1c(=O)n([C@@H]2CC[C@](C)(NC(=O)C3CC3)C2)c2cc(Nc3cc(C(C)(C)O)cc(-c4ncc(Cl)s4)n3)ncc21]

### Step 1. N-((1S,3R)-3-(6-Chloro-3-(methyl-d3)-2-oxo-2,3-dihydro-1H-imidazo[4,5-c]pyridin-1-yl)-1-methylcyclopentyl)cyclopropanecarboxamide

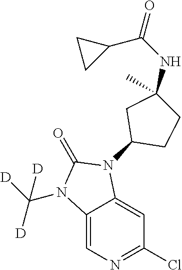 [SMILES: [2H]C([2H])([2H])n1c(=O)n([C@@H]2CC[C@](C)(NC(=O)C3CC3)C2)c2cc(Cl)ncc21]

This compound was prepared according to the procedures described in intermediate A, with tert-butyl ((1S,3R)-3-amino-1-methylcyclopentyl)carbamate replacing tert-butyl ((1R,3R)-3-aminocyclobutyl)carbamate in Step 1. LCMS calculated for C₁₇H₁₉D₃ClN₄O₂ (M+H)⁺: m/z=352.2; found: 352.2.

### Step 2. Ethyl 2-chloro-6-(4,4,5,5-tetramethyl-1,3,2-dioxaborolan-2-yl)isonicotinate

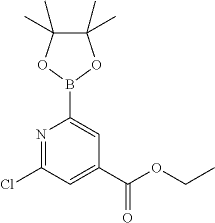 [SMILES: CCOC(=O)c1cc(Cl)nc(B2OC(C)(C)C(C)(C)O2)c1]

To a vial containing ethyl 2,6-dichloroisonicotinate (5.0 g, 22.72 mmol), 4,4,4′,4′,5,5,5′,5′-octamethyl-2,2′-bi(1,3,2-dioxaborolane) (8.66 g, 34.1 mmol), potassium acetate (4.46 g, 45.4 mmol) and Pd(dppf)₂Cl₂ dichloromethane adduct (0.928 g, 1.136 mmol) was added dioxane (33 mL) under N₂ atmosphere. The reaction was stirred at 100° C. for 5 h.

The mixture was cooled down to r.t. and filtered off the solid, and the solvent was evaporated in vacuo. To this mixture was added water (400 mL), the solid was collected and washed with MTBE. The crude material was used in the next step without further purification. LCMS calculated for C₈H₁₀BClNO₄ (M-C₆H₁₀+H)⁺: m/z=230.0; found 230.0.

### Step 3. Ethyl 2-chloro-6-(5-chlorothiazol-2-yl)isonicotinate

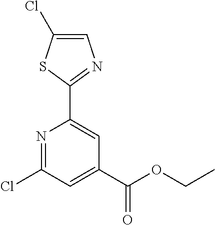 [SMILES: CCOC(=O)c1cc(Cl)nc(-c2ncc(Cl)s2)c1]

To a vial containing ethyl 2-chloro-6-(4,4,5,5-tetramethyl-1,3,2-dioxaborolan-2-yl)isonicotinate (2.355 g, 7.56 mmol), 2-bromo-5-chlorothiazole (1.0 g, 5.04 mmol), PdCl₂(dtbpf) (0.657 g, 1.008 mmol) was added THF (16.8 mL) and water (33.6 mL) followed by triethylamine (2.11 mL, 15.12 mmol) under N₂ atmosphere. The reaction was stirred at 50° C. for 3 h. The mixture was cooled down and extracted with EA. The organic layers were combined, dried over sodium sulfate, filtered, and the solvents were evaporated in vacuo. The crude material was purified by Biotage Isolera to give the product as a light yellow solid. LCMS calculated for C₁₁H₉C₁₂N₂O₂S (M+H)⁺: m/z=303.0; found 303.0.

### Step 4. 2-(2-Chloro-6-(5-chlorothiazol-2-yl)pyridin-4-yl)propan-2-ol

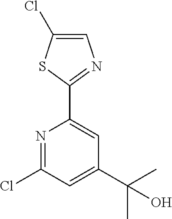 [SMILES: CC(C)(O)c1cc(Cl)nc(-c2ncc(Cl)s2)c1]

To a solution of ethyl 2-chloro-6-(5-chlorothiazol-2-yl)isonicotinate (1.0 g, 3.30 mmol) in THF (22.0 mL) was added methylmagnesium bromide (4.40 mL, 3.0 M in Et₂O) at 0° C. dropwise. The reaction was stirred at r.t. for 15 min. The mixture was quenched with sat. NH₄Cl and extracted with EA. The organic layers were combined, dried over sodium sulfate, filtered, and the solvents were evaporated in vacuo. The crude material was purified by Biotage Isolera to give the product as a light yellow solid. LCMS calculated for C₁₁H₁₁ClN₂OS (M+H)⁺: m/z=289.0; found 289.0.

### Step 5. 2-(2-Amino-6-(5-chlorothiazol-2-yl)pyridin-4-yl)propan-2-ol

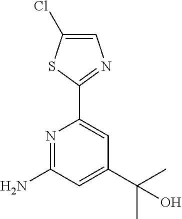 [SMILES: CC(C)(O)c1cc(N)nc(-c2ncc(Cl)s2)c1]

To a vial containing Xantphos (0.360 g, 0.622 mmol), Pd₂(dba)₃ (0.285 g, 0.311 mmol), 2-(2-chloro-6-(5-chlorothiazol-2-yl)pyridin-4-yl)propan-2-ol (0.90 g, 3.11 mmol), tert-butyl carbamate (1.1 g, 9.34 mmol), Cs₂CO₃ (3.04 g, 9.34 mmol) was added dioxane (31 mL) under N₂ atmosphere. The reaction was stirred at 100° C. overnight. The mixture was cooled down and the solid was filtered off. The solvents were evaporated in vacuo and the crude material was purified by Biotage Isolera to give tert-butyl (6-(5-chlorothiazol-2-yl)-4-(2-hydroxypropan-2-yl)pyridin-2-yl)carbamate. The material was redissolved in CH₂Cl₂ (1.0 mL) and TFA (5.0 mL). After stirring at r.t. for 1 h, the reaction mixture was concentrated in vacuo. The crude material was redissolved in CH₂Cl₂ (20 mL) and the pH of the mixture was adjusted to ˜10 with ammonia aqueous solution and then extracted into CH₂Cl₂. The organic phase was washed with brine, dried over MgSO₄ and concentrated in vacuo. The obtained crude product was used in the next step without further purification. LCMS calculated for C₁₁H₁₃ClN₃OS (M+H)⁺: m/z=270.0; found: 270.0.

### Step 6: N-((1S,3R)-3-(6-((6-(5-Chlorothiazol-2-yl)-4-(2-hydroxypropan-2-yl)pyridin-2-yl)amino)-3-(methyl-d₃)-2-oxo-2,3-dihydro-1H-imidazo[4,5-c]pyridin-1-yl)-1-methylcyclopentyl)cyclopropanecarboxamide

To a vial containing N-((1S,3R)-3-(6-amino-3-(methyl-d₃)-2-oxo-2,3-dihydro-1H-imidazo[4,5-c]pyridin-1-yl)-1-methylcyclopentyl)cyclopropanecarboxamide (15 mg, 0.045 mmol), 2-(2-amino-6-(5-chlorothiazol-2-yl)pyridin-4-yl)propan-2-ol (24 mg, 0.087 mmol), Cs₂CO₃ (85 mg, 0.262 mmol), Xantphos-Pd-G3 (16.23 mg, 0.017 mmol) was added dioxane (1.7 mL) under N₂ atmosphere. The mixture was stirred at 110° C. for 2h. The reaction mixture was cooled to r.t., diluted with MeCN and purified by prep-LCMS (XBridge C18 column, eluting with a gradient of acetonitrile/water containing 0.1% TFA, at flow rate of 60 mL/min. LCMS calculated for C₂₈H₃₀D₃ClN₇O₃S (M+H)⁺: m/z=585.2; found: 585.2. ¹H NMR (400 MHZ, DMSO-d₆) δ 10.39 (s, 1H), 8.26 (s, 1H), 8.15 (s, 1H), 8.02 (s, 1H), 7.81 (s, 1H), 7.74 (s, 1H), 7.42 (s, 1H), 4.83 (p, J=8.7 Hz, 1H), 4.17 (s, 1H), 2.41-2.21 (m, 2H), 2.20-2.05 (m, 2H), 1.91-1.72 (m, 1H), 1.65-1.50 (m, 2H), 1.47 (s, 6H), 1.38 (s, 3H), 0.62-0.55 (m, 4H).

### Example 93. Methyl ((1R,3R)-3-(6-((6-(5-chlorothiazol-2-yl)-4-(2-hydroxypropan-2-yl)pyridin-2-yl)amino)-3-(methyl-d₃)-2-oxo-2,3-dihydro-1H-imidazo[4,5-c]pyridin-1-yl)cyclopentyl)carbamate

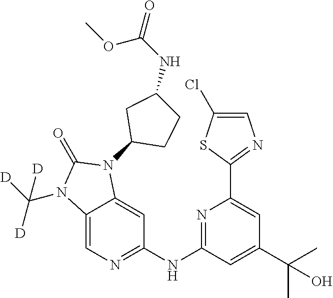 [SMILES: [2H]C([2H])([2H])n1c(=O)n([C@@H]2CC[C@@H](NC(=O)OC)C2)c2cc(Nc3cc(C(C)(C)O)cc(-c4ncc(Cl)s4)n3)ncc21]

The title compound was prepared according to the procedures described for Intermediate A and Example 92. LCMS calculated for C₂₅H₂₆D₃ClN₇O₄S (M+H)⁺: m/z=561.2; found 561.2. ¹H NMR (400 MHZ, DMSO-d₆) δ 10.52 (s, 1H), 8.28 (s, 1H), 8.03 (s, 1H), 7.76 (s, 1H), 7.62 (s, 1H), 7.45 (s, 1H), 7.33 (d, J=7.0 Hz, 1H), 4.96 (p, J=8.7 Hz, 1H), 4.20 (q, J=6.8 Hz, 1H), 3.74 (s, 1H), 3.53 (s, 3H), 2.37 (m, 1H), 2.24-2.02 (m, 3H), 1.91 (m, 1H), 1.63 (m, 1H), 1.48 (s, 6H).

### Example 94. Methyl ((1R,3R)-3-(6-((4-(2-hydroxypropan-2-yl)-6-(1-methyl-1H-pyrazol-4-yl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

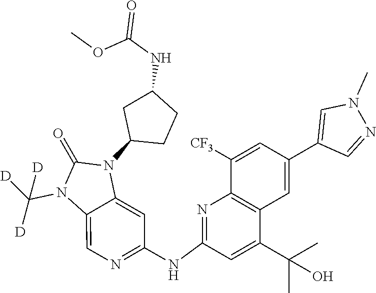 [SMILES: [2H]C([2H])([2H])n1c(=O)n([C@@H]2CC[C@@H](NC(=O)OC)C2)c2cc(Nc3cc(C(C)(C)O)c4cc(-c5cnn(C)c5)cc(C(F)(F)F)c4n3)ncc21]

### Step 1. Ethyl 2,6-dichloro-8-(trifluoromethyl)quinoline-4-carboxylate

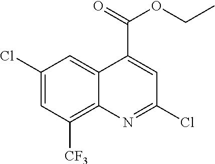 [SMILES: CCOC(=O)c1cc(Cl)nc2c(C(F)(F)F)cc(Cl)cc12]

This compound was prepared according to the procedures described in Example 3, with 5-chloro-7-(trifluoromethyl)indoline-2,3-dione replacing 7-(trifluoromethyl)indoline-2,3-dione in Step 1. LCMS calculated for C₁₃H₉C₁₂F₃NO₂ (M+H)⁺: m/z=338.0; found 338.0.

### Step 2. 2-(2,6-Dichloro-8-(trifluoromethyl)quinolin-4-yl)propan-2-ol

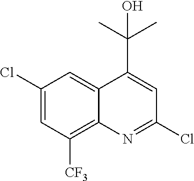 [SMILES: CC(C)(O)c1cc(Cl)nc2c(C(F)(F)F)cc(Cl)cc12]

To a solution of ethyl 2,6-dichloro-8-(trifluoromethyl)quinoline-4-carboxylate (0.34 g, 1.0 mmol) in THF (10 mL) was added methylmagnesium bromide (2.0 mL, 6.0 mmol, 3.0 M in Et₂O) at 0° C. dropwise. The reaction was stirred at r.t. for 15 min. The mixture was quenched with sat. NH₄Cl and extracted with ethyl acetate. The organic layers were combined, dried over sodium sulfate, filtered, and the solvents were evaporated in vacuo. The crude material was purified by Biotage Isolera to give the product as a light yellow solid. LCMS calculated for C₁₃H₁₁Cl₂F₃NO (M+H)⁺: m/z=324.0; found 324.0.

### Step 3. Methyl ((1R,3R)-3-(6-((6-chloro-4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

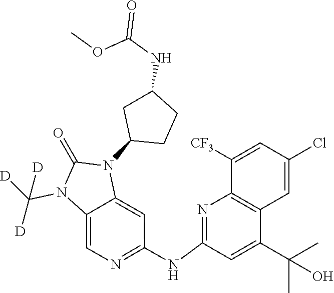 [SMILES: [2H]C([2H])([2H])n1c(=O)n([C@@H]2CC[C@@H](NC(=O)OC)C2)c2cc(Nc3cc(C(C)(C)O)c4cc(Cl)cc(C(F)(F)F)c4n3)ncc21]

This compound was prepared according to the procedures described in Example 52 with 2-(2,6-dichloro-8-(trifluoromethyl)quinolin-4-yl)propan-2-ol replacing (2-chloro-8-(trifluoromethyl)quinolin-4-yl)(morpholino)methanone in Step 5. LCMS calculated for C₂₇H₂₆D₃ClF₃N₆O₄ (M+H)⁺: m/z=596.2; found 596.2.

### Step 4. Methyl ((1R,3R)-3-(6-((4-(2-hydroxypropan-2-yl)-6-(1-methyl-1H-pyrazol-4-yl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate

To a vial containing methyl ((1R,3R)-3-(6-((6-chloro-4-(2-hydroxypropan-2-yl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate (12 mg, 0.02 mmol), (1-methyl-1H-pyrazol-4-yl)boronic acid (8 mg, 0.06 mmol), Xphos-Pd-G2 (4 mg, 0.005 mmol) and Cs₂CO₃ (20 mg, 0.06 mmol) was added dioxane (0.5 mL) and water (0.1 mL) under N₂ atmosphere. The mixture was stirred at 90° C. for 30 min. The reaction mixture was allowed to cool to r.t. diluted with MeCN and purified by prep-LCMS (XBridge C18 column, eluting with a gradient of acetonitrile/water containing 0.1% TFA, at flow rate of 60 mL/min). LCMS calculated for C₃₁H₃₁D₃F₃N₈O₄ (M+H)⁺: m/z=642.3; found 642.3. ¹H NMR (400 MHZ, DMSO-d₆) δ 11.45 (s, 1H), 9.05 (d, J=1.9 Hz, 1H), 8.39 (s, 1H), 8.37 (s, 1H), 8.30 (s, 1H), 8.02 (s, 1H), 7.52 (s, 1H), 7.37 (d, J=7.0 Hz, 1H), 5.81 (s, 1H), 4.95 (t, J=8.5 Hz, 1H), 4.26 (q, J=6.9 Hz, 1H), 4.07 (s, 1H), 3.93 (s, 3H), 3.56 (s, 3H), 2.42-2.30 (m, 1H), 2.28-1.89 (m, 4H), 1.76 (s, 6H), 1.72-1.57 (m, 1H).

### Example 95. Methyl ((1R,3R)-3-(6-((4-(2-hydroxypropan-2-yl)-6-(2-(trifluoromethyl)thiazol-5-yl)pyridin-2-yl)amino)-3-(methyl-d₃)-2-oxo-2,3-dihydro-1H-imidazo[4,5-c]pyridin-1-yl)cyclopentyl)carbamate

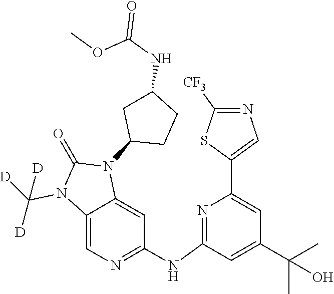 [SMILES: [2H]C([2H])([2H])n1c(=O)n([C@@H]2CC[C@@H](NC(=O)OC)C2)c2cc(Nc3cc(C(C)(C)O)cc(-c4cnc(C(F)(F)F)s4)n3)ncc21]

The title compound was prepared according to the procedures described for Intermediate A and Example 92, with 5-bromo-2-(trifluoromethyl)-thiazole as starting material. LCMS calculated for C₂₆H₂₆D₃F₃N₇O₄S (M+H)⁺: m/z=595.2; found 595.2. ¹H NMR (400 MHZ, DMSO-d₆) δ 10.39 (s, 1H), 8.86 (s, 1H), 8.24 (s, 1H), 7.78 (s, 1H), 7.51 (s, 1H), 7.44 (s, 1H), 7.30 (d, J=7.2 Hz, 1H), 4.94 (p, J=8.4 Hz, 1H), 4.18 (q, J=6.6 Hz, 1H), 3.85 (s, 1H), 3.52 (s, 3H), 2.33 (m, 1H), 2.19-2.01 (m, 3H), 1.99-1.83 (m, 1H), 1.68-1.53 (m, 1H), 1.50 (s, 6H).

### Example 96. Methyl ((1R,3R)-3-(6-((8-(2-cyanopropan-2-yl)-4-(morpholine-4-carbonyl)quinolin-2-yl)amino)-3-(methyl-d₃)-2-oxo-2,3-dihydro-1H-imidazo[4,5-c]pyridin-1-yl)cyclopentyl)carbamate

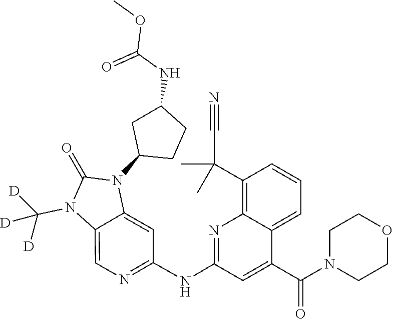 [SMILES: [2H]C([2H])([2H])n1c(=O)n([C@@H]2CC[C@@H](NC(=O)OC)C2)c2cc(Nc3cc(C(=O)N4CCOCC4)c4cccc(C(C)(C)C#N)c4n3)ncc21]

### Step 1. Methyl 8-bromo-2-chloroquinoline-4-carboxylate

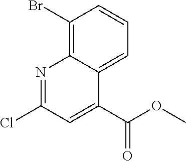 [SMILES: COC(=O)c1cc(Cl)nc2c(Br)cccc12]

This compound was prepared according to the procedure described in Example 3 (Steps 1-3), with 7-bromoindoline-2,3-dione replacing 7-(trifluoromethyl)indoline-2,3-dione in Step 1 and with methanol replacing ethanol in Step 2. LCMS calculated for C₁₁H₈BrClNO₂ (M+H)⁺: m/z=299.9; found 299.9.

### Step 2. Methyl 8-bromo-2-((2,4-dimethoxybenzyl)amino)quinoline-4-carboxylate

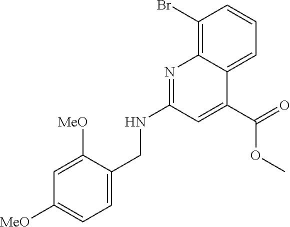 [SMILES: COC(=O)c1cc(NCc2ccc(OC)cc2OC)nc2c(Br)cccc12]

To a vial containing methyl 8-bromo-2-chloroquinoline-4-carboxylate (2.0 g, 6.7 mmol) (2,4-dimethoxyphenyl)methanamine (2.2 g, 13.3 mmol) in DMSO (19 mL) was added triethylamine (2.3 mL, 16.6 mmol), which was then stirred at 110° C. for 1 h. After cooling to room temperature, the reaction mixture was diluted with EtOAc (100 mL), washed with aqueous saturated ammonium chloride solution and then extracted into EtOAc. The organic phase was washed with brine, dried over MgSO₄ and concentrated in vacuo. The obtained crude product was purified by Biotage Isolera to give the desired product as yellow solid. LCMS calculated for C₂₀H₂₀BrN₂O₄ (M+H)⁺: m/z=431.1; found 431.1.

### Step 3. (8-Bromo-2-((2,4-dimethoxybenzyl)amino)quinolin-4-yl) (morpholino)methanone

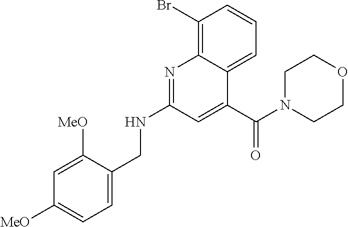 [SMILES: COc1ccc(CNc2cc(C(=O)N3CCOCC3)c3cccc(Br)c3n2)c(OC)c1]

This compound was prepared according to the procedure described in Example 3, with methyl 8-bromo-2-((2,4-dimethoxybenzyl)amino)quinoline-4-carboxylate replacing ethyl 2-((1-((1R,3R)-3-((methoxycarbonyl)amino)cyclopentyl)-3-(methyl-d₃)-2-oxo-2,3-dihydro-1H-imidazo[4,5-c]pyridin-6-yl)amino)-8-(trifluoromethyl)quinoline-4-carboxylate in Step 6 and with morpholine replacing 1-methyl-1,6-diazaspiro[3.3]heptane in Step 7. LCMS calculated for C₂₃H₂₅BrN₃O₄ (M+H)⁺: m/z=486.1; found 486.1.

### Step 4. tert-Butyl (8-bromo-4-(morpholine-4-carbonyl)quinolin-2-yl)(2,4-dimethoxybenzyl)carbamate

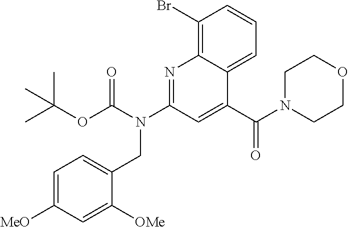 [SMILES: COc1ccc(CN(C(=O)OC(C)(C)C)c2cc(C(=O)N3CCOCC3)c3cccc(Br)c3n2)c(OC)c1]

To a reaction vial containing (8-bromo-2-((2,4-dimethoxybenzyl)amino)quinolin-4-yl)(morpholino)methanone (575 mg, 1.18 mmol), DMAP (43 mg, 0.35 mmol) were added CH₂Cl₂ (11.8 mL) and di-tert-butyl dicarbonate (0.4 mL, 1.77 mmol). The reaction mixture was allowed to stir at r.t. for 1 h, after which it was quenched with saturated ammonium chloride solution and extracted with EtOAc. The organic layers were combined, dried over sodium sulfate, filtered, and the solvents were evaporated in vacuo. The obtained crude product was purified by Biotage Isolera to give the desired product as yellow solid. LCMS calculated for C₂₈H₃₃BrN₃O₆ (M+H)⁺: m/z=586.2; found 586.2.

### Step 5. tert-Butyl (8-(cyanomethyl)-4-(morpholine-4-carbonyl) quinolin-2-yl)(2,4-dimethoxybenzyl)carbamate

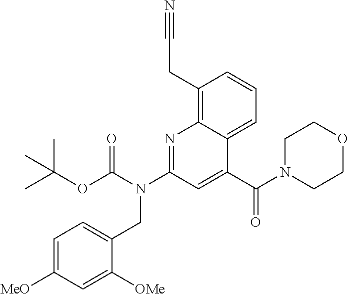 [SMILES: COc1ccc(CN(C(=O)OC(C)(C)C)c2cc(C(=O)N3CCOCC3)c3cccc(CC#N)c3n2)c(OC)c1]

A reaction vial containing sodium cyanoacetate (42 mg, 0.39 mmol), SPhos Pd G4 (15.6 mg, 0.02 mmol), and tert-butyl (8-bromo-4-(morpholine-4-carbonyl)quinolin-2-yl)(2,4-dimethoxybenzyl)carbamate (115 mg, 0.20 mmol) was evacuated and backfilled with nitrogen. Mesitylene (1.0 mL) was added to the reaction mixture, which was then stirred at 140° C. for 18 h. The reaction mixture was cooled to r.t and the solvents were evaporated in vacuo. The obtained crude product was purified by Biotage Isolera to give the desired product as brown solid. LCMS calculated for C₃₀H₃₅N₄O₆ (M+H)⁺: m/z=547.3; found 547.3.

### Step 6. tert-Butyl (8-(2-cyanopropan-2-yl)-4-(morpholine-4-carbonyl)quinolin-2-yl) (2,4-dimethoxybenzyl)carbamate

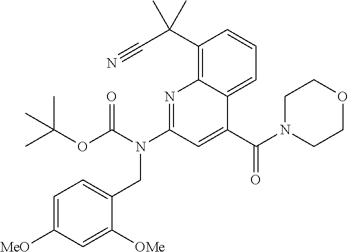 [SMILES: COc1ccc(CN(C(=O)OC(C)(C)C)c2cc(C(=O)N3CCOCC3)c3cccc(C(C)(C)C#N)c3n2)c(OC)c1]

To a reaction vial containing tert-butyl (8-(cyanomethyl)-4-(morpholine-4-carbonyl)quinolin-2-yl)(2,4-dimethoxybenzyl)carbamate (40 mg, 0.07 mmol) and sodium hydride (42 mg, 0.29 mmol, 60% dispersion in mineral oil) in THF (2.0 mL) was added iodomethane (18 μL, 0.29 mmol) dropwise. The reaction mixture was stirred at 50° C. for 18 h. The reaction mixture was quenched with water and extracted with EtOAc and the organic layer was subsequently washed with water and brine, dried over sodium sulfate, filtered, and the solvents were evaporated in vacuo. The crude material was purified by Biotage Isolera to give the product. LCMS calculated for C₃₂H₃₉N₄O₆ (M+H)⁺: m/z=575.3; found 575.3.

### Step 7. 2-(2-Amino-4-(morpholine-4-carbonyl)quinolin-8-yl)-2-methylpropanenitrile

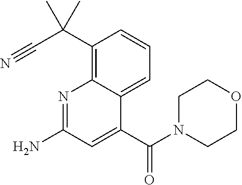 [SMILES: CC(C)(C#N)c1cccc2c(C(=O)N3CCOCC3)cc(N)nc12]

To a vial containing tert-butyl (8-(2-cyanopropan-2-yl)-4-(morpholine-4-carbonyl)quinolin-2-yl)(2,4-dimethoxybenzyl)carbamate (20 mg, 0.03 mmol) was added TFA (2.0 mL) and stirred at 100° C. for 10 min. The mixture was cooled to r.t., concentrated in vacuo and azeotroped with MeCN. The crude material was used in the next step without further purification. LCMS calculated for C₁₈H₂₁N₄O₂ (M+H)⁺: m/z=325.2; found 325.2.

### Step 8. Methyl ((1R,3R)-3-(6-((8-(2-cyanopropan-2-yl)-4-(morpholine-4-carbonyl)quinolin-2-yl)amino)-3-(methyl-d₃)-2-oxo-2,3-dihydro-1H-imidazo[4,5-c]pyridin-]-yl)cyclopentyl)carbamate

This compound was prepared according to the procedure described in Example 3, with 2-(2-amino-4-(morpholine-4-carbonyl)quinolin-8-yl)-2-methylpropanenitrile replacing ethyl 2-amino-8-(trifluoromethyl)quinoline-4-carboxylate in Step 5. LCMS calculated for C₃₂H₃₄D₃N₈O₅ (M+H)⁺: m/z=616.3; found 616.3.

### Example 97. Methyl ((1R,3R)-3-(6-((3-(1-(difluoromethyl)-1H-pyrazol-4-yl)-2-methyl-8-(4-(trifluoromethyl)pyridin-3-yl)imidazo[1,2-b]pyridazin-6-yl)amino)-3-(methyl-d₃)-2-oxo-2,3-dihydro-1H-imidazo[4,5-c]pyridin-1-yl)cyclopentyl-3-d)carbamate

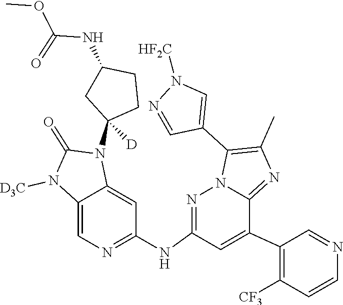

### Step 1. 6-Chloro-2-methyl-8-(4-(trifluoromethyl)pyridin-3-yl)imidazo[1,2-b]pyridazine

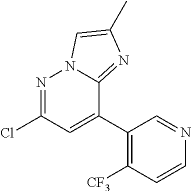 [SMILES: Cc1cn2nc(Cl)cc(-c3cnccc3C(F)(F)F)c2n1]

A vial containing 3-(4,4,5,5-tetramethyl-1,3,2-dioxaborolan-2-yl)-4-(trifluoromethyl)pyridine (1.33 g, 4.87 mmol), 8-bromo-6-chloro-2-methylimidazo[1,2-b]pyridazine (1.00 g, 4.06 mmol), (1,1′-bis(diphenylphosphino)ferrocene)-dichloropalladium(II) dichloromethane adduct (331 mg, 0.406 mmol) and potassium carbonate (1.12 g, 8.11 mmol) was evacuated and backfilled with nitrogen three times, followed by the addition of 1,4-dioxane (18.7 mL) and water (1.5 mL) and purging with nitrogen. The vial was sealed and heated to 80° C. for 2 h. After cooling to room temperature, the mixture was filtered through a SiliaPrep SPE thiol cartridge (SPE-R51030B-06P) and washed with CH₂Cl₂. The mixture was washed with brine, dried over MgSO₄ and concentrated in vacuo. The obtained crude product was purified by Biotage Isolera to give the desired product as yellow solid. LCMS calculated for C₁₃H₉ClF₃N₄ (M+H)⁺: m/z=313.0; found: 313.1.

### Step 2. tert-Butyl (2-methyl-8-(4-(trifluoromethyl)pyridin-3-yl)imidazo[1,2-b]pyridazin-6-yl)carbamate

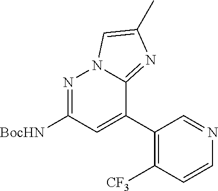 [SMILES: Cc1cn2nc(NC(=O)OC(C)(C)C)cc(-c3cnccc3C(F)(F)F)c2n1]

A vial containing 6-chloro-2-methyl-8-(4-(trifluoromethyl)pyridin-3-yl)imidazo[1,2-b]pyridazine (600 mg, 1.92 mmol), tert-butyl carbamate (674 mg, 5.76 mmol), cesium carbonate (1.25 g, 3.84 mmol), 4,5-bis(diphenylphosphino)-9,9-dimethylxanthene (222 mg, 0.384 mmol) and tris(dibenzylideneacetone)dipalladium(0) (176 mg, 0.192 mmol) was evacuated and backfilled with nitrogen three times, followed by the addition of dioxane (9.6 mL). The vial was sealed and heated to 100° C. for 6 h. After cooling to r.t., the mixture was filtered through a SiliaPrep SPE thiol cartridge (SPE-R51030B-06P) and washed with CH₂Cl₂. The mixture was washed with brine, dried over MgSO₄ and concentrated in vacuo. The obtained crude product was purified by Biotage Isolera to give the desired product as brown solid. LCMS calculated for C₁₈H₁₉F₃N₅O₂ (M+H)⁺: m/z=394.1; found 394.1.

### Step 3. tert-Butyl (3-iodo-2-methyl-8-(4-(trifluoromethyl)pyridin-3-yl)imidazo[1,2-b]pyridazin-6-yl)carbamate

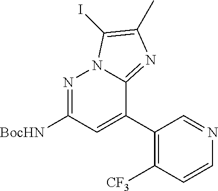 [SMILES: Cc1nc2c(-c3cnccc3C(F)(F)F)cc(NC(=O)OC(C)(C)C)nn2c1I]

To a vial containing tert-butyl (2-methyl-8-(4-(trifluoromethyl)pyridin-3-yl)imidazo[1,2-b]pyridazin-6-yl)carbamate (500 mg, 1.71 mmol) and N-iodosuccinimide (384 mg, 1.71 mmol) was added acetonitrile (8.5 mL). The reaction mixture was heated to 80° C. for 15 min. After cooling to r.t., the mixture was concentrated in vacuo. The obtained crude product was purified by Biotage Isolera to give the desired product as brown solid. LCMS calculated for C₁₈H₁₈F₃IN₅O₂ (M+H)⁺: m/z=520.0; found 520.1.

### Step 4. 3-(1-(Difluoromethyl)-1H-pyrazol-4-yl)-2-methyl-8-(4-(trifluoromethyl)pyridin-3-yl)imidazo[1,2-b]pyridazin-6-amine

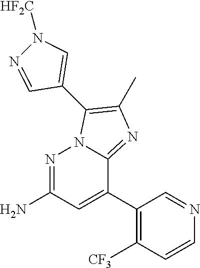

A vial containing 1-(difluoromethyl)-4-(4,4,5,5-tetramethyl-1,3,2-dioxaborolan-2-yl)-1H-pyrazole (611 mg, 2.51 mmol), tert-butyl (3-iodo-2-methyl-8-(4-(trifluoromethyl)pyridin-3-yl)imidazo[1,2-b]pyridazin-6-yl)carbamate (700 mg, 1.67 mmol), (1,1′-bis(diphenylphosphino)ferrocene)-dichloropalladium(II) dichloromethane adduct (136 mg, 0.167 mmol) and potassium carbonate (462 mg, 3.34 mmol) was evacuated and backfilled with nitrogen three times, followed by the addition of 1,4-dioxane (15.2 mL) and water (1.5 mL) and purging with nitrogen. The vial was sealed and heated to 80° C. for 2 h. After cooling to room temperature, the mixture was filtered through a SiliaPrep SPE thiol cartridge (SPE-R51030B-06P) and washed with CH₂Cl₂. The mixture was washed with brine, dried over MgSO₄ and concentrated in vacuo. The crude material was redissolved in CH₂Cl₂ (5.0 mL) and TFA (5.0 mL). After stirring at 40° C. for 1 h, the reaction mixture was concentrated in vacuo. The crude material was redissolved in CH₂Cl₂ (20 mL) and the pH of the mixture was adjusted to ˜10 with ammonia aqueous solution and then extracted into CH₂Cl₂. The organic phase was washed with brine, dried over MgSO₄ and concentrated in vacuo. The obtained crude product was purified by Biotage Isolera to give the desired product as light yellow solid. LCMS calculated for C₁₇H₁₃F₅N₇ (M+H)⁺: m/z=410.1; found 410.1.

### Step 5. tert-Butyl ((IR)-3-aminocyclopentyl-3-d)carbamate

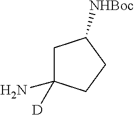 [SMILES: [2H]C1(N)CC[C@@H](NC(=O)OC(C)(C)C)C1]

To a vial containing tert-butyl (R)-(3-oxocyclopentyl)carbamate (0.50 g, 2.51 mmol) and ammonium formate (0.791 g, 12.55 mmol) was added MeOH (5.0 mL). The reaction mixture was stirred at 45° C. for 30 min. Then sodium cyanoborodeuteride (0.38 g, 5.77 mmol) was added to the reaction mixture. After stirring at 45° C. for 5 h, the reaction mixture was concentrated in vacuo. The obtained crude product was used in the next step without further purification as a mixture of two diastereomers. LCMS calculated for C₁₀H₂₀DN₂O₂ (M+H)⁺: m/z=202.2; found: 202.1.

### Step 6. Methyl ((1R)-3-(6-chloro-3-(methyl-d₃)-2-oxo-2,3-dihydro-1H-imidazo[4,5-c]pyridin-1-yl)cyclopentyl-3-d)carbamate

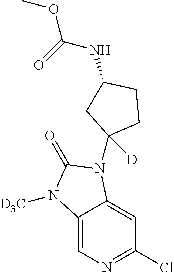 [SMILES: [2H]C([2H])([2H])n1c(=O)n(C2([2H])CC[C@@H](NC(=O)OC)C2)c2cc(Cl)ncc21]

This compound was prepared according to the procedures described in Intermediate A as a mixture of two diastereomers, with tert-butyl ((1R)-3-aminocyclopentyl-3-d)carbamate replacing tert-butyl ((1R,3R)-3-aminocyclopentyl)carbamate in Step 1 and iodomethane-d₃ replacing iodomethane in Step 4. LCMS calculated for C₁₄H₁₄D₄ClN₄O₃ (M+H)⁺: m/z=329.1; found: 329.1.

### Step 7. Methyl ((1R,3R)-3-(6-((3-(1-(difluoromethyl)-1H-pyrazol-4-yl)-2-methyl-8-(4-(trifluoromethyl)pyridin-3-yl)imidazo[1,2-b]pyridazin-6-yl)amino)-3-(methyl-d₃)-2-oxo-2,3-dihydro-1H-imidazo[4,5-c]pyridin-1-yl)cyclopentyl-3-d)carbamate

This compound was prepared according to the procedures described in Example 53, with methyl ((1R)-3-(6-chloro-3-(methyl-d₃)-2-oxo-2,3-dihydro-1H-imidazo[4,5-c]pyridin-1-yl)cyclopentyl-3-d)carbamate replacing methyl ((1R,3R)-3-(6-chloro-3-methyl-2-oxo-2,3-dihydro-1H-imidazo[4,5-c]pyridin-1-yl)cyclopentyl)carbamate and 3-(1-(difluoromethyl)-1H-pyrazol-4-yl)-2-methyl-8-(4-(trifluoromethyl)pyridin-3-yl)imidazo[1,2-b]pyridazin-6-amine replacing methyl (3-amino-5-(1-methyl-1H-pyrazol-4-yl)phenyl)carbamate in Step 4 as the first peak of two regioisomers. LCMS calculated for C₃₁H₂₅D₄F₅N₁₁O₃ (M+H)⁺: m/z=702.3; found: 702.3. ¹H NMR (500 MHz, DMSO) δ 10.25 (s, 1H), 9.06 (d, J=5.2 Hz, 1H), 8.95 (s, 1H), 8.86 (s, 1H), 8.55 (s, 1H), 8.16 (s, 1H), 8.03 (d, J=5.2 Hz, 1H), 7.94 (t, ²J_{H-F}=58.9 Hz, 1H), 7.53-7.45 (m, 2H), 7.25 (d, J=7.3 Hz, 1H), 4.23-4.15 (m 1H), 3.53 (s, 3H), 2.50 (s, 3H), 2.24 (dd, J=13.5, 7.9 Hz, 1H), 2.13-2.05 (m, 2H), 2.01-1.93 (m, 1H), 1.87 (dd, J=13.5, 5.7 Hz, 1H), 1.60-1.53 (m, 1H).

### Example 98. Methyl ((1R,3R)-3-(6-((5-methoxy-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl)carbamate

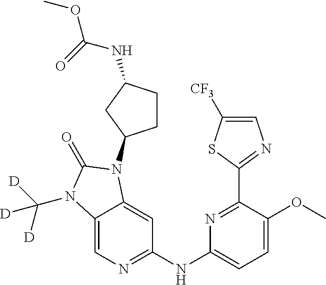 [SMILES: [2H]C([2H])([2H])n1c(=O)n([C@@H]2CC[C@@H](NC(=O)OC)C2)c2cc(Nc3ccc(OC)c(-c4ncc(C(F)(F)F)s4)n3)ncc21]

### Step 1. 5-Methoxy-6-(5-(trifluoromethyl)thiazol-2-yl)pyridin-2-amine

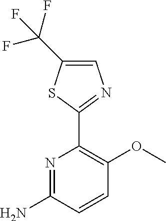 [SMILES: COc1ccc(N)nc1-c1ncc(C(F)(F)F)s1]

A vial containing 6-bromo-5-methoxypyridin-2-amine (15 mg, 0.07 mmol) and [1,1′-bis(diphenylphosphino)ferrocene]-dichloropalladium(II) (6 mg, 7.4 μmol) was evacuated and backfilled with nitrogen. Tetrahydrofuran (1.0 mL) and (5-(trifluoromethyl)thiazol-2-yl)zinc(II) bromide (443 μL, 0.09 mmol, 0.2 M) (Example 105, Step 1) were added to the reaction mixture, which was then stirred at 50° C. for 2 h. The reaction mixture was cooled to r.t. and was diluted with EtOAc. The organic layer was subsequently washed with water and brine, dried over sodium sulfate, filtered, and the solvents were evaporated in vacuo. The crude material was purified by Biotage Isolera to give the product. LCMS calculated for C₁₀H₉F₃N₃OS (M+H)⁺: m/z=276.0; found 276.0.

### Step 2. Methyl ((1R,3R)-3-(6-((5-methoxy-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl)carbamate

This compound was prepared according to the procedures described in Example 32, Step 2 with 5-methoxy-6-(5-(trifluoromethyl)thiazol-2-yl)pyridin-2-amine replacing 6-(1-methyl-1H-pyrazol-4-yl)quinolin-2-amine and with methyl ((1R,3R)-3-(6-chloro-3-(methyl-d₃)-2-oxo-2,3-dihydro-1H-imidazo[4,5-c]pyridin-1-yl)cyclopentyl)carbamate (Example 75, Step 1) replacing methyl ((1R,3R)-3-(6-chloro-3-methyl-2-oxo-2,3-dihydro-1H-imidazo[4,5-c]pyridin-1-yl)cyclopentyl)carbamate. LCMS calculated for C₂₄H₂₂D₃F₃N₂O₄S (M+H)⁺: m/z=567.2; found: 567.2.

### Example 99. Methyl ((1R,3R)-3-(6-((6-(5-(2-hydroxypropan-2-yl)thiazol-2-yl)-4-methylpyridin-2-yl)amino)-3-(methyl-d₃)-2-oxo-2,3-dihydro-1H-imidazo[4,5-c]pyridin-1-yl)cyclopentyl)carbamate

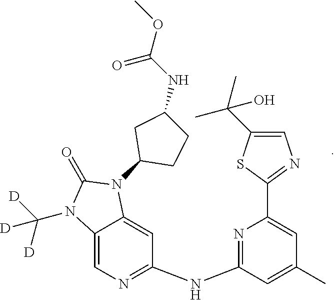 [SMILES: [2H]C([2H])([2H])n1c(=O)n([C@@H]2CC[C@@H](NC(=O)OC)C2)c2cc(Nc3cc(C)cc(-c4ncc(C(C)(C)O)s4)n3)ncc21]

The title compound was prepared according to the procedures described for Intermediate A, Example 91, Example 94, and Example 98, with ethyl 2-bromothiazole-5-carboxylate and 2-bromo-6-chloro-4-methylpyridine as starting materials. LCMS calculated for C₂₆H₂₉D₃N₇O₄S (M+H)⁺: m/z=541.2; found: 541.2. ¹H NMR (600 MHz, DMSO) δ 10.46 (s, 1H), 8.22 (s, 1H), 7.78 (s, 1H), 7.66 (s, 1H), 7.57 (s, 1H), 7.24 (d, J=7.1 Hz, 1H), 7.12 (s, 1H), 5.73 (s, 1H), 4.98-4.90 (m, 1H), 4.28-4.19 (m, 1H), 3.53 (s, 3H), 2.40 (s, 3H), 2.32 (ddd, J=14.7, 7.3, 7.3 Hz, 1H), 2.20-2.12 (m, 2H), 2.12-2.04 (m, 1H), 1.94 (ddd, J=14.5, 9.6, 5.8 Hz, 1H), 1.63-1.60 (m, 1H), 1.60 (s, 6H).

### Example 100. Methyl ((1R,3R)-3-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl)carbamate

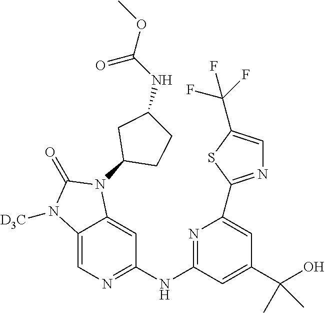 [SMILES: [2H]C([2H])([2H])n1c(=O)n([C@@H]2CC[C@@H](NC(=O)OC)C2)c2cc(Nc3cc(C(C)(C)O)cc(-c4ncc(C(F)(F)F)s4)n3)ncc21]

The title compound was prepared according to the procedures described for Intermediate A and Example 92, with 2-bromo-5-(trifluoromethyl)-thiazole and methyl 2-chloro-isonicotinate as starting materials. LCMS calculated for C₂₆H₂₆D₃F₃N₇O₄S (M+H)⁺: m/z=595.2; found 595.2. ¹H NMR (600 MHz, DMSO-d₆) δ 10.58 (s, 1H), 8.60 (s, 1H), 8.29 (s, 1H), 7.89 (s, 1H), 7.58 (s, 1H), 7.50 (s, 1H), 7.30 (d, J=7.1 Hz, 1H), 5.50 (s, 1H), 4.96 (p, J=8.5 Hz, 1H), 4.18 (q, J=6.9 Hz, 1H), 3.51 (s, 3H), 2.34 (m, 1H), 2.17-2.12 (m, 1H), 2.12-2.04 (m, 2H), 1.89 (m, 1H), 1.57 (s, 1H), 1.49 (s, 6H).

### Example 101. Methyl ((1R,3R)-3-(6-((4-((3-(difluoromethyl)azetidin-1-yl)methyl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl)carbamate

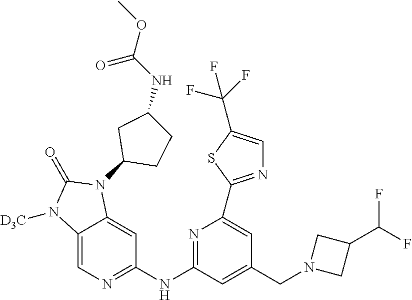 [SMILES: [2H]C([2H])([2H])n1c(=O)n([C@@H]2CC[C@@H](NC(=O)OC)C2)c2cc(Nc3cc(CN4CC(C(F)F)C4)cc(-c4ncc(C(F)(F)F)s4)n3)ncc21]

### Step 1. (2-Chloro-6-(5-(trifluoromethyl)thiazol-2-yl)pyridin-4-yl)methanol

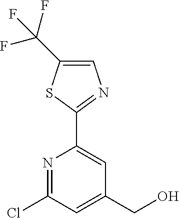 [SMILES: OCc1cc(Cl)nc(-c2ncc(C(F)(F)F)s2)c1]

To a reaction vial containing methyl 2-chloro-6-(5-(trifluoromethyl)thiazol-2-yl)isonicotinate (550 mg, 1.704 mmol; according to Example 92, Step 2) in THF (5.0 mL) was added lithium borohydride (187 mg, 8.52 mmol) portion wise, which was then stirred at r.t. for 1 h. The reaction mixture was quenched with saturated NaHCO₃ aq. solution and extracted with EtOAc. The organic layer was dried over sodium sulfate, filtered, and the solvents were evaporated in vacuo. The crude material was purified by Biotage Isolera. LCMS calculated for C₁₀H₇ClF₃N₂OS (M+H)⁺: m/z=295.0; found 294.9.

### Step 2. Methyl ((1R,3R)-3-(6-((4-(hydroxymethyl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl)carbamate

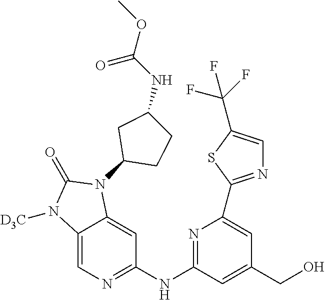 [SMILES: [2H]C([2H])([2H])n1c(=O)n([C@@H]2CC[C@@H](NC(=O)OC)C2)c2cc(Nc3cc(CO)cc(-c4ncc(C(F)(F)F)s4)n3)ncc21]

This compound was prepared according to the procedure described in Example 18 (Step 5) using (2-chloro-6-(5-(trifluoromethyl)thiazol-2-yl)pyridin-4-yl)methanol instead of 6-chloro-8-(trifluoromethyl)quinolin-2-amine. LCMS calculated for C₂₄H₂₂D₃F₃N₇O₄S (M+H)⁺: m/z=567.2; found 567.2.

### Step 3. Methyl ((1R,3R)-3-(6-((4-(chloromethyl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl)carbamate

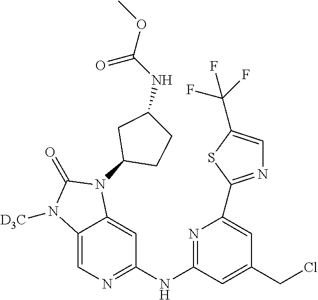 [SMILES: [2H]C([2H])([2H])n1c(=O)n([C@@H]2CC[C@@H](NC(=O)OC)C2)c2cc(Nc3cc(CCl)cc(-c4ncc(C(F)(F)F)s4)n3)ncc21]

To a reaction vial containing methyl ((1R,3R)-3-(6-((4-(hydroxymethyl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl)carbamate (28 mg, 0.049 mmol) was added thionyl chloride (0.4 mL, 5.48 mmol), which was then stirred at r.t. for 12 h. The reaction mixture was concentrated, quenched with saturated NaHCO₃ aq. solution and extracted with DCM. The organic layer was dried over sodium sulfate, filtered, and the solvents were evaporated in vacuo. The crude material was used directly for next step. LCMS calculated for C₂₄H₂₁D₃ClF₃N₇O₃S (M+H)⁺: m/z=585.1; found 585.2.

### Step 4. Methyl ((1R,3R)-3-(6-((4-((3-(difluoromethyl)azetidin-1-yl)methyl)-6-(5-(trifluoromethyl) thiazol-2-yl)pyridin-2-yl)amino)-3-(methyl-d₃)-2-oxo-2,3-dihydro-1H-imidazo[4,5-c]pyridin-1-yl)cyclopentyl)carbamate

To a reaction vial containing methyl ((1R,3R)-3-(6-((4-(chloromethyl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl)carbamate (15 mg, 0.026 mmol) and cesium carbonate (33 mg, 0.103 mmol) in DMF (0.5 mL) was added 3-(difluoromethyl)azetidine hydrochloride (5 mg, 0.051 mmol), which was then stirred at 80° C. for 1 h. The reaction mixture was allowed to cool to r.t., diluted with MeCN and purified by prep-LCMS (XBridge C18 column, eluting with a gradient of acetonitrile/water containing 0.1% TFA, at flow rate of 60 mL/min). LCMS calculated for C₂₈H₂₇D₃F₅N₈O₃S (M+H)⁺: m/z=656.2; found 656.2.

### Example 102. Methyl ((1R,3R)-3-(6-((6-cyclobutoxy-5-(1-methyl-1H-pyrazol-4-yl)-4-(morpholine-4-carbonyl)pyridin-2-yl)amino)-3-(methyl-d₃)-2-oxo-2,3-dihydro-1H-imidazo[4,5-c]pyridin-1-yl)cyclopentyl)carbamate

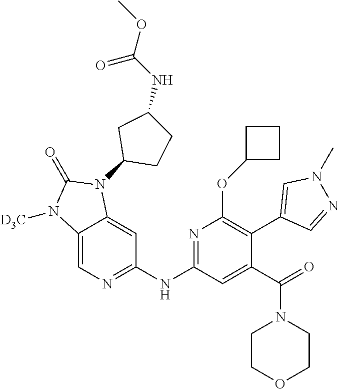 [SMILES: [2H]C([2H])([2H])n1c(=O)n([C@@H]2CC[C@@H](NC(=O)OC)C2)c2cc(Nc3cc(C(=O)N4CCOCC4)c(-c4cnn(C)c4)c(OC4CCC4)n3)ncc21]

### Step 1. (2,6-Dichloropyridin-4-yl)(morpholino)methanone

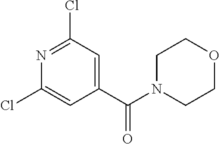 [SMILES: O=C(c1cc(Cl)nc(Cl)c1)N1CCOCC1]

This compound was prepared according to the procedure described in Example 52 using 2,6-dichloroisonicotinic acid instead of 2-chloro-8-(trifluoromethyl)quinoline-4-carboxylic acid in Step 3. LCMS calculated for C₁₀H₁₁Cl₂N₂O₂ (M+H)⁺: m/z=261.0; found 261.1.

### Step 2. (2-Chloro-6-cyclobutoxypyridin-4-yl)(morpholino)methanone

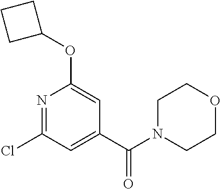 [SMILES: O=C(c1cc(Cl)nc(OC2CCC2)c1)N1CCOCC1]

To a reaction vial containing (2,6-dichloropyridin-4-yl)(morpholino)methanone (600 mg, 2.3 mmol) and cyclobutanol (166 mg, 2.3 mmol) in DMF (3.0 mL) was added sodium hydride (176 mg, 4.6 mmol, 60% dispersion in mineral oil) portion wise. The reaction mixture was stirred at r.t. for 1 h. The reaction mixture was quenched with water and extracted with EtOAc and the organic layer was subsequently washed with water and brine, dried over sodium sulfate, filtered, and the solvents were evaporated in vacuo. The crude material was purified by Biotage Isolera to give the product. LCMS calculated for C₁₄H₁₈ClN₂O₃ (M+H)⁺: m/z=297.1; found 297.1.

### Step 3. (2-Amino-6-cyclobutoxypyridin-4-yl) (morpholino)methanone

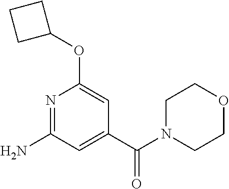 [SMILES: Nc1cc(C(=O)N2CCOCC2)cc(OC2CCC2)n1]

A reaction vial containing (2-chloro-6-cyclobutoxypyridin-4-yl)(morpholino)methanone (307 mg, 1.04 mmol), benzophenone imine (375 mg, 2.07 mmol), tris(dibenzylideneacetone)dipalladium(0) (189 mg, 0.21 mmol), Xantphos (239 mg, 0.41 mmol) and cesium carbonate (674 mg, 2.07 mmol) was evacuated and backfilled with nitrogen. 1,4-Dioxane (3 mL) was added to the reaction mixture, which was then stirred at 80° C. overnight. The reaction mixture was cooled to r.t and diluted with EtOAc. The organic layer was subsequently washed with water and brine, dried over sodium sulfate, filtered, and the solvents were evaporated in vacuo. The residue was dissolved in MeOH (5 mL) and treated with hydroxylammonium chloride (288 mg, 4.14 mmol) and potassium acetate (508 mg, 5.17 mmol). The mixture was stirred at r.t. for 1h before it was filtered and concentrated. The crude material was purified by Biotage Isolera. LCMS calculated for C₁₄H₂₀N₃O₃ (M+H)⁺: m/z=278.1; found 278.1.

### Step 4. (6-Amino-3-bromo-2-cyclobutoxypyridin-4-yl)(morpholino)methanone

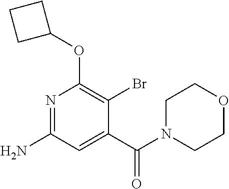 [SMILES: Nc1cc(C(=O)N2CCOCC2)c(Br)c(OC2CCC2)n1]

To a cooled reaction vial containing (2-amino-6-cyclobutoxypyridin-4-yl)(morpholino)methanone (440 mg, 1.59 mmol) in MeOH (5 mL) was added N-bromosuccinimide (85 mg, 0.48 mmol) portion wise. The reaction mixture was stirred at 0° C. for 1 h. The reaction mixture was quenched with water and extracted with EtOAc and the organic layer was subsequently washed with water and brine, dried over sodium sulfate, filtered, and the solvents were evaporated in vacuo. The crude material was purified by Biotage Isolera to give the product. LCMS calculated for C₁₄H₁₉BrN₃O₃ (M+H)⁺: m/z=356.1; found 356.1.

### Step 5. (6-Amino-2-cyclobutoxy-3-(1-methyl-1H-pyrazol-4-yl)pyridin-4-yl) (morpholino)methanone

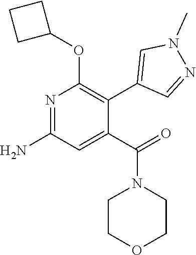 [SMILES: Cn1cc(-c2c(C(=O)N3CCOCC3)cc(N)nc2OC2CCC2)cn1]

A reaction vial containing (6-amino-3-bromo-2-cyclobutoxypyridin-4-yl)(morpholino)-methanone (46 mg, 0.129 mmol), 1-methyl-4-(4,4,5,5-tetramethyl-1,3,2-dioxaborolan-2-yl)-1H-pyrazole (40 mg, 0.194 mmol), XPhos-Pd-G2 (20 mg, 0.026 mmol) and cesium carbonate (84 mg, 0.258 mmol) was evacuated and backfilled with nitrogen. MeCN (1 mL) and water (0.2 mL) was added to the reaction mixture, which was then stirred at 70° C. for 1 h. The reaction mixture was allowed to cool to r.t. and the solvents were evaporated in vacuo. The crude material was purified by Biotage Isolera to give the product. LCMS calculated for C₁₈H₂₄N₅O₃ (M+H)⁺: m/z=358.2; found 358.2.

### Step 6. Methyl (1R,3R)-3-(6-(6-cyclobutoxy-5-(1-methyl-1H-pyrazol-4-yl)-4-(morpholine-4-carbonyl)pyridin-2-yl)amino)-3-(methyl-d₃)-2-oxo-2,3-dihydro-1H-imidazo[4,5-c]pyridin-1-yl)cyclopentyl)carbamate

A reaction vial containing methyl ((1R,3R)-3-(6-chloro-3-(methyl-d3)-2-oxo-2,3-dihydro-1H-imidazo[4,5-c]pyridin-1-yl)cyclopentyl)carbamate (15 mg, 0.046 mmol), (6-amino-2-cyclobutoxy-3-(1-methyl-1H-pyrazol-4-yl)pyridin-4-yl)(morpholino)methanone (16 mg, 0.046 mmol), tBuBrettPhos-Pd-G3 (8 mg, 0.009 mmol) and sodium tert-butoxide (18 mg, 0.183 mmol) was evacuated and backfilled with nitrogen. THF (0.5 mL) was added to the reaction mixture, which was then stirred at r.t. for 1 h. The reaction mixture was diluted with MeCN and purified by prep-LCMS (XBridge C18 column, eluting with a gradient of acetonitrile/water containing 0.1% TFA, at flow rate of 60 mL/min). LCMS calculated for C₃₂H₃₇D₃N₉O₆ (M+H)⁺: m/z=649.3; found 649.4.

### Example 103. Methyl ((1R,3R)-3-(6-((2-(5-(difluoromethyl)thiophen-2-yl)-6-(hydroxymethyl)pyrimidin-4-yl)amino)-3-(methyl-d₃)-2-oxo-2,3-dihydro-1H-imidazo[4,5-c]pyridin-1-yl)cyclopentyl)carbamate

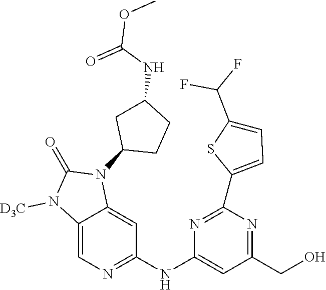 [SMILES: [2H]C([2H])([2H])n1c(=O)n([C@@H]2CC[C@@H](NC(=O)OC)C2)c2cc(Nc3cc(CO)nc(-c4ccc(C(F)F)s4)n3)ncc21]

The title compound was prepared according to the procedures described for Intermediate A, Example 63, Example 67, and Example 102, with 2-(5-(difluoromethyl)-thiophen-2-yl)-4,4,5,5-tetramethyl-1,3,2-dioxaborolane and 6-amino-2-chloropyrimidine-4-carboxylate as starting materials. LCMS calculated for C₂₄H₂₃D₃F₂N₇O₄S (M+H)⁺: m/z=549.2; found 549.2.

### Example 104. Methyl ((1R,3R)-3-(6-((6-(5-(difluoromethyl)thiophen-2-yl)-4-(morpholine-4-carbonyl)pyridin-2-yl)amino)-3-(methyl-d₃)-2-oxo-2,3-dihydro-1H-imidazo[4,5-c]pyridin-1-yl)cyclopentyl)carbamate

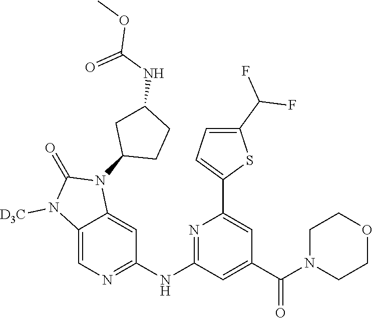 [SMILES: [2H]C([2H])([2H])n1c(=O)n([C@@H]2CC[C@@H](NC(=O)OC)C2)c2cc(Nc3cc(C(=O)N4CCOCC4)cc(-c4ccc(C(F)F)s4)n3)ncc21]

The title compound was prepared according to the procedures described for Intermediate A, Example 63 and Example 102. LCMS calculated for C₂₉H₂₉D₃F₂N₇O₅S (M+H)⁺: m/z=631.2; found 631.4.

### Example 105. Methyl ((1R,3R)-3-(6-((4-(1-acetylpiperidin-4-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl)carbamate

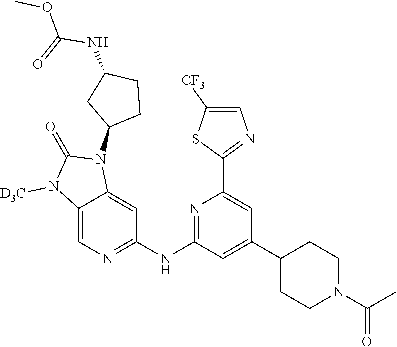 [SMILES: [2H]C([2H])([2H])n1c(=O)n([C@@H]2CC[C@@H](NC(=O)OC)C2)c2cc(Nc3cc(C4CCN(C(C)=O)CC4)cc(-c4ncc(C(F)(F)F)s4)n3)ncc21]

### Step 1. (5-(Trifluoromethyl)thiazol-2-yl)zinc(II) bromide

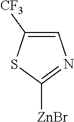

To a vial containing acid-treated (0.1 M HCl washed and dried) zinc dust (0.981 g, 15.00 mmol) in THF (4.2 mL) was added 1,2-dibromoethane (22 μL, 0.250 mmol). The mixture was heated at 65° C. for 5 min under nitrogen, then cool down to room temperature. This 1,2-dibromoethane (0.022 mL, 0.250 mmol) addition-reflux-cool down cycle was repeated two times. Then trimethylchlorosilane (0.064 mL, 0.500 mmol) was added at r.t. and stirred for 15 min. After the activation of zinc, this vial was evacuated and backfilled with nitrogen three times. Then 2-bromo-5-(trifluoromethyl)thiazole (1.16 g, 5.00 mmol) was dissolved in 1.0 mL of THF and added to the zinc suspension dropwise. The reaction was heated at reflux until the full consumption of 2-bromo-5-(trifluoromethyl)thiazole confirmed by LC-MS. The concentration of (5-(trifluoromethyl)thiazol-2-yl)zinc(II) bromide was determined by the titration with iodine solution in THF.

### Step 2. 4-Chloro-6-(5-(trifluoromethyl) thiazol-2-yl)pyridin-2-amine

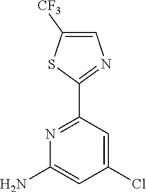 [SMILES: Nc1cc(Cl)cc(-c2ncc(C(F)(F)F)s2)n1]

To a vial containing 6-bromo-4-chloropyridin-2-amine (500 mg, 2.410 mmol) and (1,1′-bis(diphenylphosphino)ferrocene)-dichloropalladium(II) dichloromethane adduct (197 mg, 0.241 mmol) in THF (10.0 mL) was added (5-(trifluoromethyl)thiazol-2-yl)zinc(II) bromide solution (6.0 mL, 0.4 M in THF) via a syringe. The reaction mixture was heated at 75° C. for 2 h. After cooling to room temperature, the mixture was filtered through a SiliaPrep SPE thiol cartridge (SPE-R51030B-06P) and washed with CH₂Cl₂. The mixture was washed with brine, dried over MgSO₄ and concentrated in vacuo. The obtained crude product was purified by Biotage Isolera to give the desired product as yellow solid. LCMS calculated for C₉H₆ClF₃N₃S (M+H)⁺: m/z=280.0; found 280.0.

### Step 3. tert-Butyl 4-(2-amino-6-(5-(trifluoromethyl)thiazol-2-yl)pyridin-4-yl)piperidine-1-carboxylate

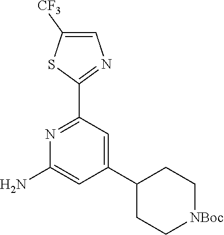 [SMILES: CC(C)(C)OC(=O)N1CCC(c2cc(N)nc(-c3ncc(C(F)(F)F)s3)c2)CC1]

A vial containing 4-chloro-6-(5-(trifluoromethyl)thiazol-2-yl)pyridin-2-amine (75 mg. 0.268 mmol), tert-butyl 4-(4,4,5,5-tetramethyl-1,3,2-dioxaborolan-2-yl)-3,6-dihydropyridine-1(2H)-carboxylate (166 mg, 0.536 mmol), potassium phosphate tribasic (114 mg, 0.536 mmol) and chloro(2-dicyclohexylphosphino-2′,4′,6′-triisopropyl-1, l′-biphenyl)[2-(2′-amino-1,1′-biphenyl)]palladium(II) (21 mg, 27 μmol) was evacuated and backfilled with nitrogen three times, followed by the addition of 1,4-dioxane (3.0 mL) and water (0.3 mL) and purging with nitrogen. The vial was sealed and heated to 100° C. for 2 h. After cooling to room temperature, the mixture was filtered through a SiliaPrep SPE thiol cartridge (SPE-R51030B-06P) and washed with CH₂Cl₂. The mixture was washed with brine, dried over MgSO₄ and concentrated in vacuo. The crude product was dissolved in MeOH (5.0 mL), followed by the addition of Pd/C (10 wt %, 30 mg). The vial was purged with hydrogen gas for 5 min. and then stirred for 2 h under an atmosphere of hydrogen at 50° C. After cooling to room temperature, the reaction mixture was filtered through Celite and washed with CH₂Cl₂, followed by concentration of the filtrate in vacuo. The resulting residue was purified by Biotage Isolera to give the desired product as an off-white solid. LCMS calculated for C₁₉H₂₄F₃N₄O₂S (M+H)⁺: m/z=429.2; found 429.2.

### Step 4. Methyl ((1R,3R)-3-(3-(methyl-d₃)-2-oxo-6-((4-(piperidin-4-yl)-6-(5-(trifluoromethyl)thiazol-2-yl)pyridin-2-yl)amino)-2,3-dihydro-1H-imidazo[4,5-c]pyridin-1-yl)cyclopentyl)carbamate

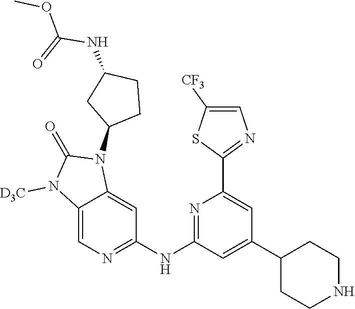 [SMILES: [2H]C([2H])([2H])n1c(=O)n([C@@H]2CC[C@@H](NC(=O)OC)C2)c2cc(Nc3cc(C4CCNCC4)cc(-c4ncc(C(F)(F)F)s4)n3)ncc21]

A vial containing methyl ((1R,3R)-3-(6-chloro-3-(methyl-d₃)-2-oxo-2,3-dihydro-1H-imidazo[4,5-c]pyridin-1-yl)cyclopentyl)carbamate (30 mg, 92 μmol), tert-butyl 4-(2-amino-6-(5-(trifluoromethyl)thiazol-2-yl)pyridin-4-yl)piperidine-1-carboxylate (47 mg, 0.110 mmol), tris(dibenzylideneacetone)dipalladium(0) (17 mg, 18 μmol), 4,5-bis(diphenylphosphino)-9,9-dimethylxanthene (21 mg, 37 μmol) and cesium carbonate (60 mg, 0.183 mmol) was evacuated and backfilled with nitrogen three times, followed by the addition of 1,4-dioxane (1 mL). The vial was sealed and heated to 100° C. for 3 h. After cooling to room temperature, the mixture was filtered through a SiliaPrep SPE thiol cartridge (SPE-R51030B-06P) and washed with CH₂Cl₂. The mixture was washed with brine, dried over MgSO₄ and concentrated in vacuo. The crude material was redissolved in CH₂Cl₂ (2.0 mL) and TFA (2.0 mL). After stirring at 40° C. for 1 h, the reaction mixture was concentrated in vacuo. The crude material was redissolved in CH₂Cl₂ (20 mL) and the pH of the mixture was adjusted to ˜10 with ammonia aqueous solution and then extracted into CH₂Cl₂. The organic phase was washed with brine, dried over MgSO₄ and concentrated in vacuo. The obtained crude product was used in the next step without further purification. LCMS calculated for C₂₈H₂₉D₃F₃N₈O₃S (M+H)⁺: m/z=620.2; found 620.2.

### Step 5. Methyl ((1R,3R)-3-(6-((4-(1-acetylpiperidin-4-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl)carbamate

To a vial containing methyl ((1R,3R)-3-(3-(methyl-d₃)-2-oxo-6-((4-(piperidin-4-yl)-6-(5-(trifluoromethyl)thiazol-2-yl)pyridin-2-yl)amino)-2,3-dihydro-1H-imidazo[4,5-c]pyridin-1-yl)cyclopentyl)carbamate (10 mg, 16 μmol), DIPEA (14 μl, 81 μmol), DMAP (1 mg, 8.1 μmol) in CH₂Cl₂ (0.5 mL) was added acetic anhydride (5 μl, 48 μmol). After stirring at r.t. for 30 min, the reaction mixture was quenched with water (0.5 mL). The mixture was then diluted with acetonitrile and purified with prep-LCMS (XBridge C18 column, eluting with a gradient of acetonitrile/water containing 0.1% TFA, at flow rate of 60 mL/min). LCMS calculated for C₃₀H₃₁D₃F₃N₈O₄S (M+H)⁺: m/z=662.3; found: 662.3. ¹H NMR (400 MHZ, DMSO) δ 10.24 (s, 1H), 8.59 (d, J=1.4 Hz, 1H), 8.22 (s, 1H), 7.69 (s, 1H), 7.61 (s, 1H), 7.37 (s, 1H), 7.28 (d, J=7.0 Hz, 1H), 4.94 (p, J=8.4 Hz, 1H), 4.56 (d, J=13.5 Hz, 1H), 4.21-4.13 (m, 1H), 3.96 (d, J=14.2 Hz, 2H), 3.51 (s, 3H), 3.18 (t, J=12.9 Hz, 1H), 2.94 (t, J=12.1 Hz, 1H), 2.70-2.59 (m, 1H), 2.34 (m, 1H), 2.18-2.07 (m, 2H), 2.06 (s, 3H), 1.95-1.82 (m, 3H), 1.72-1.40 (m, 3H).

### Example 106. 6-((3-(1-(Difluoromethyl)-1H-pyrazol-4-yl)-2-methyl-8-(4-(trifluoromethyl)pyridin-3-yl)imidazo[1,2-b]pyridazin-6-yl)amino)-1-((1r,3r)-3-(2-hydroxypropan-2-yl)cyclobutyl)-3-(methyl-d₃)-1,3-dihydro-2H-imidazo[4,5-c]pyridin-2-one

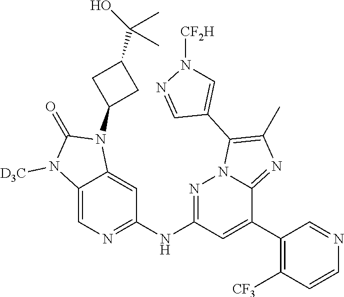 [SMILES: [2H]C([2H])([2H])n1c(=O)n([C@H]2C[C@H](C(C)(C)O)C2)c2cc(Nc3cc(-c4cnccc4C(F)(F)F)c4nc(C)c(-c5cnn(C(F)F)c5)n4n3)ncc21]

The title compound was prepared according to the procedures described for Intermediate A, Example 92, and Example 97, with methyl (1r,3r)-3-aminocyclobutane-1-carboxylate hydrochloride as starting material. LCMS calculated for C₃₁H₂₇D₃F₅N₁₀O₂ (M+H)⁺: m/z=672.3; found 672.2. ¹H NMR (400 MHz, DMSO) δ 10.25 (s, 1H), 9.06 (d, J=5.2 Hz, 1H), 8.95 (d, J=2.3 Hz, 1H), 8.90 (s, 1H), 8.52 (s, 1H), 8.16 (d, J=3.4 Hz, 1H), 8.03 (d, J=5.3 Hz, 1H), 7.94 (t, ²J_{H-F}=58.9 Hz, 1H), 7.60 (d, J=5.0 Hz, 1H), 7.47 (s, 1H), 4.86-4.67 (m, 1H), 2.96-2.81 (m, 1H), 2.75-2.59 (m, 2H), 2.50 (s, 3H), 2.41-2.27 (m, 2H), 1.07 (s, 6H).

### Example 107. Methyl ((1R,3R)-3-(6-((6-(5-(2-hydroxypropan-2-yl)thiazol-2-yl)-4-(1-methyl-1H-pyrazol-4-yl)pyridin-2-yl)amino)-3-(methyl-d₃)-2-oxo-2,3-dihydro-1H-imidazo[4,5-c]pyridin-1-yl)cyclopentyl)carbamate

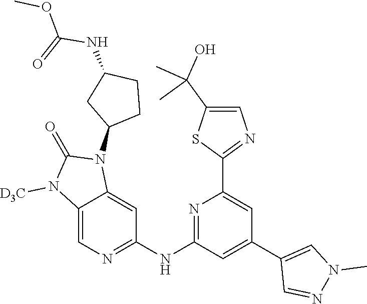

The title compound was prepared according to the procedures described for Intermediate A, Example 92, and Example 105, with ethyl 2-bromothiazole-5-carboxylate and 1-methyl-4-(4,4,5,5-tetramethyl-1,3,2-dioxaborolan-2-yl)-1H-pyrazole as starting materials. LCMS calculated for C₂₉H₃₁D₃N₉O₄S (M+H)⁺: m/z=607.3; found 607.3.

### Example 108. Methyl ((1R,3R)-3-(3-(methyl-d₃)-2-oxo-6-((6-(4-(trifluoromethyl)-1H-pyrazol-1-yl)pyridin-2-yl)amino)-2,3-dihydro-1H-imidazo[4,5-c]pyridin-1-yl)cyclopentyl)carbamate

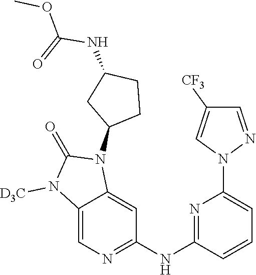 [SMILES: [2H]C([2H])([2H])n1c(=O)n([C@@H]2CC[C@@H](NC(=O)OC)C2)c2cc(Nc3cccc(-n4cc(C(F)(F)F)cn4)n3)ncc21]

### Step 1. 2-Chloro-6-(4-(trifluoromethyl)-1H-pyrazol-1-yl)pyridine

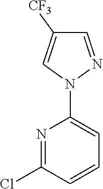 [SMILES: FC(F)(F)c1cnn(-c2cccc(Cl)n2)c1]

To a vial containing 2-chloro-6-nitropyridine (80 mg, 0.505 mmol), 4-(trifluoromethyl)-1H-pyrazole (69 mg, 0.505 mmol) and cesium carbonate (164 mg, 0.505 mmol) was added 1,4-dioxane (1.2 mL). After stirring at 100° C. for 16 h, the reaction mixture was quenched with aqueous sat. ammonium chloride solution (2 mL) and extracted with CH₂Cl₂. The organic mixture was washed with brine, dried over MgSO₄ and concentrated in vacuo. The resulting residue was purified by Biotage Isolera to give the desired product as an off-white solid. LCMS calculated for C₉H₆ClF₃N₃ (M+H)⁺: m/z=248.0; found 248.1.

### Step 2. Methyl ((1R,3R)-3-(3-(methyl-d₃)-2-oxo-6-((6-(4-(trifluoromethyl)-1H-pyrazol-1-yl)pyridin-2-yl)amino)-2,3-dihydro-1H-imidazo[4,5-c]pyridin-1-yl)cyclopentyl)carbamate

To a vial containing methyl ((1R,3R)-3-(6-amino-3-(methyl-d₃)-2-oxo-2,3-dihydro-1H-imidazo[4,5-c]pyridin-1-yl)cyclopentyl)carbamate (12 mg, 39 μmol), 2-chloro-6-(4-(trifluoromethyl)-1H-pyrazol-1-yl)pyridine (12 mg, 47 μmol), tris(dibenzylideneacetone)dipalladium(0) (5 mg, 5.8 μmol), 4,5-bis(diphenylphosphino)-9,9-dimethylxanthene (7 mg, 12 μmol) and cesium carbonate (25 mg. 78 μmol) was added 1,4-dioxane (0.4 mL). The vial was sealed and heated to 100° C. for 4 h. After cooling to room temperature, the mixture was filtered through a SiliaPrep SPE thiol cartridge (SPE-R51030B-06P) and washed with acetonitrile. The mixture was then diluted with acetonitrile and purified with prep-LCMS (XBridge C18 column, eluting with a gradient of acetonitrile/water containing 0.1% TFA, at flow rate of 60 mL/min). LCMS calculated for C₂₃H₂₁D₃F₃N₈O₃ (M+H)⁺: m/z=520.2; found 520.2. ¹H NMR (400 MHz, DMSO) δ 10.33 (s, 1H), 9.02 (s, 1H), 8.30 (s, 1H), 8.17 (s, 1H), 7.95 (t, J=7.9 Hz, 1H), 7.52-7.37 (m, 3H), 7.32 (d, J=7.0 Hz, 1H), 4.96 (p, J=8.5 Hz, 1H), 4.21-4.15 (m, 1H), 3.53 (s, 3H), 2.34-2.24 (m, 1H), 2.20-1.99 (m, 3H), 1.95-1.83 (m, 1H), 1.65-1.51 (m, 1H).

### Example 109. Methyl ((1R,3R)-3-(3-(methyl-d₃)-2-oxo-6-((1-(tetrahydro-2H-pyran-4-yl)-3-(1-(trifluoromethyl)-1H-pyrazol-4-yl)-1H-pyrrolo[3,2-b]pyridin-5-yl)amino)-2,3-dihydro-1H-imidazo[4,5-c]pyridin-1-yl)cyclopentyl)carbamate

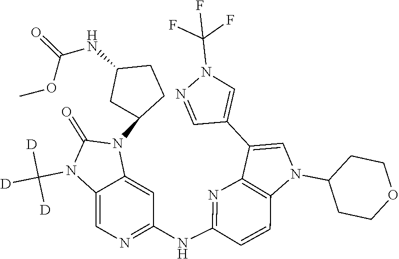 [SMILES: [2H]C([2H])([2H])n1c(=O)n([C@@H]2CC[C@@H](NC(=O)OC)C2)c2cc(Nc3ccc4c(n3)c(-c3cnn(C(F)(F)F)c3)cn4C3CCOCC3)ncc21]

### Step 1. 5-Chloro-1-((2-(trimethylsilyl)ethoxy)methyl)-1H-pyrrolo[3,2-b]pyridine

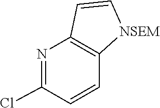 [SMILES: C[Si](C)(C)CCOCn1ccc2nc(Cl)ccc21]

To a flask containing 5-chloro-1H-pyrrolo[3,2-b]pyridine (5.1 g, 33 mmol) as a solution in DMF (25 mL) at 0° C. was added NaH (60% dispersion in mineral oil, 1.6 g, 40 mmol), and the reaction mixture was stirred at 0° C. After 30 min, SEM-C₁ (7.7 mL, 43 mmol) was added, and the reaction was stirred at rt. After 1 h, the reaction was quenched with water and the product was extracted with EtOAc, washed with brine, dried over MgSO₄, filtered, and concentrated in vacuo. The crude product was used directly in the next step without further purification. LCMS calculated for C₁₃H₂₀ClN₂OSi (M+H)⁺: m/z=283.1; found 283.1.

### Step 2. 5-Chloro-3-iodo-1-((2-(trimethylsilyl)ethoxy)methyl)-1H-pyrrolo[3,2-b]pyridine

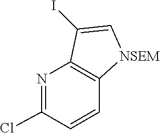 [SMILES: C[Si](C)(C)CCOCn1cc(I)c2nc(Cl)ccc21]

This compound was prepared according to the procedures described in Example 130, with 5-chloro-1-((2-(trimethylsilyl)ethoxy)methyl)-1H-pyrrolo[3,2-b]pyridine replacing 7-bromo-5-chloro-1-((2-(trimethylsilyl)ethoxy)methyl)-1H-pyrrolo[3,2-b]pyridine in Step 2. LCMS calculated for C₁₃H₁₉ClIN₂OSi (M+H)⁺: m/z=409.0; found: 409.1.

### Step 3. 5-Chloro-3-(1-(trifluoromethyl)-1H-pyrazol-4-yl)-1-((2-(trimethylsilyl)ethoxy)methyl)-1H-pyrrolo[3,2-b]pyridine

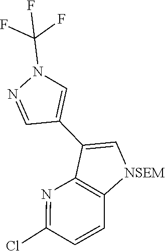 [SMILES: C[Si](C)(C)CCOCn1cc(-c2cnn(C(F)(F)F)c2)c2nc(Cl)ccc21]

This compound was prepared according to the procedures described in Example 130, with 5-chloro-3-iodo-1-((2-(trimethylsilyl)ethoxy)methyl)-1H-pyrrolo[3,2-b]pyridine replacing 7-bromo-5-chloro-3-iodo-1-((2-(trimethylsilyl)ethoxy)methyl)-1H-pyrrolo[3,2-b]pyridine in Step 3. LCMS calculated for C₁₇H₂₁ClF₃N₄OSi (M+H)⁺: m/z=417.1; found: 417.1.

### Step 4. 3-(1-(Trifluoromethyl)-1H-pyrazol-4-yl)-1-((2-(trimethylsilyl)ethoxy)methyl)-1H-pyrrolo[3,2-b]pyridin-5-amine

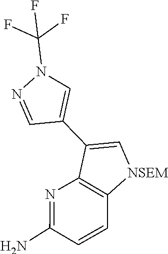 [SMILES: C[Si](C)(C)CCOCn1cc(-c2cnn(C(F)(F)F)c2)c2nc(N)ccc21]

A reaction vial containing 5-chloro-3-(1-(trifluoromethyl)-1H-pyrazol-4-yl)-1-((2-(trimethylsilyl)ethoxy)methyl)-1H-pyrrolo[3,2-b]pyridine (1.2 g, 2.9 mmol), diphenylmethanimine (1.4 mL, 8.6 mmol), tris(dibenzylideneacetone)dipalladium(0) (260 mg, 0.29 mmol), 9,9-dimethyl-4,5-bis(diphenylphosphino)xanthene (330 mg, 0.58 mmol) and Cs₂CO₃ (2.8 g, 8.7 mmol) was evacuated and backfilled with nitrogen three times, followed by the addition of 1,4-dioxane (5 mL). The mixture was heated to 105° C. overnight. After cooling to room temperature, the mixture was filtered through Celite and washed with CH₂Cl₂, followed by concentration of the filtrate in vacuo. The crude residue was dissolved in MeOH (20 mL) and then hydroxylamine hydrochloride (260 mg, 3.7 mmol) and potassium acetate (367 mg, 3.7 mmol) were added. The reaction mixture was left to stir at rt. After 5 h, the reaction mixture was concentrated in vacuo and the product was extracted by EtOAc three times, dried over MgSO₄, and concentrated in vacuo. The resulting residue was purified by Teledyne ISCO CombiFlash to give the desired product (900 mg, 79% yield). LCMS calculated for C₁₇H₂₃F₃N₅OSi (M+H)⁺: m/z=398.2; found: 398.1.

### Step 5. Methyl ((1R,3R)-3-(3-(methyl-d₃)-2-oxo-6-((3-(1-(trifluoromethyl)-1H-pyrazol-4-yl)-1H-pyrrolo[3,2-b]pyridin-5-yl)amino)-2,3-dihydro-1H-imidazo[4,5-c]pyridin-1-yl)cyclopentyl)carbamate

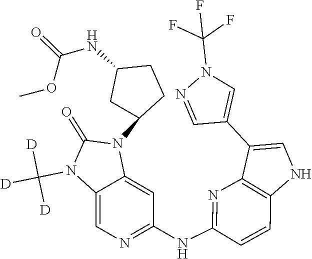 [SMILES: [2H]C([2H])([2H])n1c(=O)n([C@@H]2CC[C@@H](NC(=O)OC)C2)c2cc(Nc3ccc4[nH]cc(-c5cnn(C(F)(F)F)c5)c4n3)ncc21]

A reaction vial containing methyl ((1R,3R)-3-(6-chloro-3-(methyl-d₃)-2-oxo-2,3-dihydro-1H-imidazo[4,5-c]pyridin-1-yl)cyclopentyl)carbamate (750 mg, 2.3 mmol), 3-(1-(trifluoromethyl)-1H-pyrazol-4-yl)-1-((2-(trimethylsilyl)ethoxy)methyl)-1H-pyrrolo[3,2-b]pyridin-5-amine (900 mg, 2.3 mmol), tris(dibenzylideneacetone)dipalladium(0) (210 mg, 0.23 mmol), 9,9-dimethyl-4,5-bis(diphenylphosphino)xanthene (264 mg, 0.46 mmol) and Cs₂CO₃ (2.2 g, 6.8 mmol) was evacuated and backfilled with nitrogen three times, followed by the addition of 1,4-dioxane (5 mL). The reaction mixture was heated to 105° C. overnight. After cooling to room temperature, the mixture was filtered through Celite and washed with CH₂C₁₂, followed by concentration of the filtrate in vacuo. The resulting residue was purified by Teledyne ISCO CombiFlash to give the desired intermediate (850 mg, 54% yield). The purified material was then dissolved in 60% TFA in CH₂Cl₂ and stirred at rt. After 2 h, the reaction mixture was concentrated in vacuo and to the crude residue was added 1:1:1 THF/MeOH/NH₄OH solution (10 mL) and left to stir at rt. After 30 min, the mixture was extracted with 3:1 CHCl₃/iso-propanol, dried over Na₂SO₄ and concentrated in vacuo to give the product as a brown solid. LCMS calculated for C₂H₂₂D₃F₃N₉O₃ (M+H)⁺: m/z=559.2; found: 559.2.

### Step 6. Methyl ((1R,3R)-3-(3-(methyl-d₃)-2-oxo-6-((1-(tetrahydro-2H-pyran-4-yl)-3-(1-(trifluoromethyl)-1H-pyrazol-4-yl)-1H-pyrrolo[3,2-b]pyridin-5-yl)amino)-2,3-dihydro-1H-imidazo[4,5-c]pyridin-1-yl)cyclopentyl)carbamate

To a vial containing methyl ((1R,3R)-3-(3-(methyl-d₃)-2-oxo-6-((3-(1-(trifluoromethyl)-1H-pyrazol-4-yl)-1H-pyrrolo[3,2-b]pyridin-5-yl)amino)-2,3-dihydro-1H-imidazo[4,5-c]pyridin-1-yl)cyclopentyl)carbamate (170 mg, 0.3 mmol) and Cs₂CO₃ (198 mg, 0.6 mmol) as a suspension in MeCN (2 mL) was added by 4-iodotetrahydro-2H-pyran (129 mg, 0.6 mmol). The reaction was heated to 90° C. overnight. After cooling to room temperature, the mixture was filtered through Celite and washed with CH₂Cl₂, followed by concentration of the filtrate in vacuo. The crude residue was dissolved in MeCN and purified by prep-LCMS (XBridge C18 column, eluting with a gradient of acetonitrile/water containing 0.1% TFA, at flow rate of 60 mL/min). LCMS calculated for C₃₀H₃₀D₃F₃N₉O₄ (M+H)⁺: m/z=643.3; found: 643.3. ¹H NMR (400 MHz, DMSO-d₆) δ 11.08 (s, 1H), 8.82 (s, 1H), 8.52 (s, 1H), 8.39-8.27 (m, 2H), 8.10 (s, 1H), 7.34 (d, J=5.0 Hz, 1H), 7.44-7.00 (m, 2H), 5.00-4.88 (m, 1H), 4.81-4.70 (m, 1H), 4.25-4.14 (m, 1H), 4.10-4.01 (m, 2H), 3.64-3.56 (m, 2H), 3.54 (s, 3H), 2.33-2.24 (m, 1H), 2.21-1.96 (m, 7H), 1.96-1.88 (m, 1H), 1.66-1.56 (m, 1H).

### Example 110. Methyl ((1R,3R)-3-(6-((1-(2-hydroxy-2-methylpropyl)-3-(1-(trifluoromethyl)-1H-pyrazol-4-yl)-1H-pyrrolo[3,2-b]pyridin-5-yl)amino)-3-(methyl-d₃)-2-oxo-2,3-dihydro-1H-imidazo[4,5-c]pyridin-1-yl)cyclopentyl)carbamate

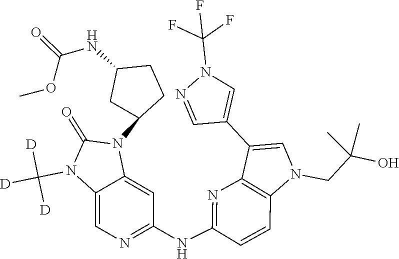 [SMILES: [2H]C([2H])([2H])n1c(=O)n([C@@H]2CC[C@@H](NC(=O)OC)C2)c2cc(Nc3ccc4c(n3)c(-c3cnn(C(F)(F)F)c3)cn4CC(C)(C)O)ncc21]

The title compound was prepared according to the procedures described in Example 109, using 2,2-dimethyloxirane as starting material in Step 6. LCMS calculated for C₂₉H₃₀D₃F₃N₉O₄ (M+H)⁺: m/z=631.3.; found: 631.3.

### Example 111. Methyl ((3R)-3-(3-(methyl-d₃)-2-oxo-6-((1-(tetrahydro-2H-pyran-4-yl)-3-(1-(trifluoromethyl)-1H-pyrazol-4-yl)-1H-pyrrolo[3,2-b]pyridin-5-yl)amino)-2,3-dihydro-1H-imidazo[4,5-c]pyridin-1-yl)cyclopentyl-1-d)carbamate

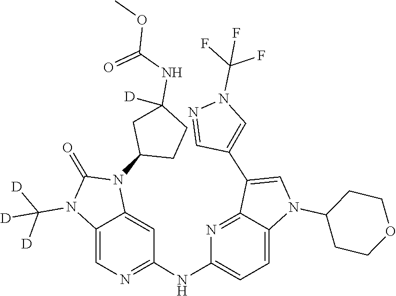 [SMILES: [2H]C1(NC(=O)OC)CC[C@@H](n2c(=O)n(C([2H])([2H])[2H])c3cnc(Nc4ccc5c(n4)c(-c4cnn(C(F)(F)F)c4)cn5C4CCOCC4)cc32)C1]

The title compound was prepared according to the procedures described in Example 109, using methyl ((3R)-3-(6-chloro-3-(methyl-d₃)-2-oxo-2,3-dihydro-1H-imidazo[4,5-c]pyridin-1-yl)cyclopentyl-1-d)carbamate (Example 158, Step 5) as starting material in Step 5. Purified by prep-LCMS (XBridge C18 column, eluting with a gradient of 23%>41% acetonitrile in water containing 0.1% TFA, at flow rate of 60 mL/min over 12 min). The desired diastereomer was collected at a retention time of 10.7 min. LCMS calculated for C₃₀H₂₉D₄F₃N₉O₄ (M+H)⁺: m/z=644.3.; found: 644.3.

### Example 112. Methyl ((3R)-3-(3-(methyl-d₃)-2-oxo-6-((1-(tetrahydro-2H-pyran-4-yl-4-d)-3-(1-(trifluoromethyl)-1H-pyrazol-4-yl)-1H-pyrrolo[3,2-b]pyridin-5-yl)amino)-2,3-dihydro-1H-imidazo[4,5-c]pyridin-1-yl)cyclopentyl-1-d)carbamate

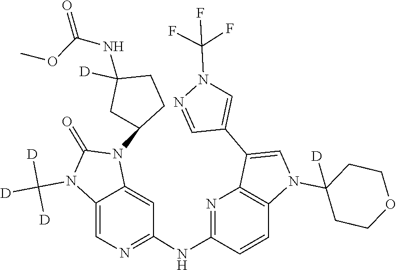 [SMILES: [2H]C1(NC(=O)OC)CC[C@@H](n2c(=O)n(C([2H])([2H])[2H])c3cnc(Nc4ccc5c(n4)c(-c4cnn(C(F)(F)F)c4)cn5C4([2H])CCOCC4)cc32)C1]

### Step 1. Tetrahydro-2H-pyran-4-d-4-ol

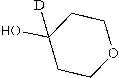

To a vial containing tetrahydro-4H-pyran-4-one (2 g, 20 mmol) in MeOH (20 mL) at 0° C. was added sodium tetrahydroborate-d₄ (1 g, 24 mmol) in three portions. The reaction was stirred at rt for 2 h and the reaction mixture was concentrated in vacuo. The crude residue was dissolved in CH₂Cl₂, diluted with sat. aq. NaHCO₃ and extracted with CH₂Cl₂. The organic layer was dried over Na₂SO₄, filtered, and concentrated in vacuo. The crude residue was used directly in the next step without further purification.

### Step 2. Tetrahydro-2H-pyran-4-yl-4-d 4-methylbenzenesulfonate

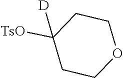 [SMILES: [2H]C1(OS(=O)(=O)c2ccc(C)cc2)CCOCC1]

To a vial containing tetrahydro-2H-pyran-4-d-4-ol (500 mg, 4.8 mmol) and triethylamine (1.3 mL, 9.7 mmol) in CH₂Cl₂ (5 mL) at 0° C. was added TsCl (920 mg, 4.8 mmol). The reaction was stirred at rt for 2 h and then quenched with sat. aq. NaHCO₃ solution and extracted twice with CH₂Cl₂. The organic layer was dried over Na₂SO₄, filtered, and concentrated in vacuo. The resulting residue was purified by Teledyne ISCO CombiFlash to give the desired product.

### Step 3. Methyl (3R)-3-(3-(methyl-d₃)-2-oxo-6-(1-(tetrahydro-2H-pyran-4-yl-4-d)-3-(1-(trifluoromethyl)-1H-pyrazol-4-yl)-1H-pyrrolo[3,2-b]pyridin-5-yl)amino)-2,3-dihydro-1H-imidazo[4,5-c]pyridin-1-yl)cyclopentyl-1-d)carbamate

This compound was prepared according to the procedures described in Example 109, with methyl ((3R)-3-(6-chloro-3-(methyl-d₃)-2-oxo-2,3-dihydro-1H-imidazo[4,5-c]pyridin-1-yl)cyclopentyl-1-d)carbamate replacing methyl ((1R,3R)-3-(6-chloro-3-(methyl-d₃)-2-oxo-2,3-dihydro-1H-imidazo[4,5-c]pyridin-1-yl)cyclopentyl)carbamate in Step 5 and tetrahydro-2H-pyran-4-yl-4-d 4-methylbenzenesulfonate replacing 4-iodotetrahydro-2H-pyran in Step 6 and was purified by prep-LCMS (XBridge C18 column, eluting with a gradient of 24%→42% acetonitrile in water containing 0.1% TFA, at flow rate of 60 mL/min over 12 min). The desired diastereomer was collected at a retention time of 9.5 min. LCMS calculated for C₃₀H₂₈D₅F₃N₉O₄ (M+H)⁺: m/z=645.3.; found: 645.3.

### Example 113. Methyl ((3R)-3-(6-((1-(1-cyanocyclopropyl)-3-(1-(trifluoromethyl)-1H-pyrazol-4-yl)-1H-pyrrolo[3,2-b]pyridin-5-yl)amino)-3-(methyl-d₃)-2-oxo-2,3-dihydro-1H-imidazo[4,5-c]pyridin-1-yl)cyclopentyl-1-d)carbamate

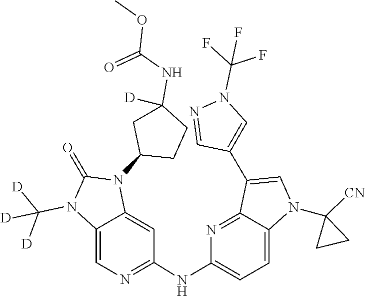 [SMILES: [2H]C1(NC(=O)OC)CC[C@@H](n2c(=O)n(C([2H])([2H])[2H])c3cnc(Nc4ccc5c(n4)c(-c4cnn(C(F)(F)F)c4)cn5C4(C#N)CC4)cc32)C1]

The title compound was prepared according to the procedures described in to Example 109, using methyl ((3R)-3-(6-chloro-3-(methyl-d₃)-2-oxo-2,3-dihydro-1H-imidazo[4,5-c]pyridin-1-yl)cyclopentyl-1-d)carbamate (Example 158, Step 5) as starting material in Step 5 and 1-bromocyclopropane-1-carbonitrile as starting material in Step 6. Purified by prep-LCMS (XBridge C18 column, eluting with a gradient of 23→41% acetonitrile in water containing 0.1% TFA, at flow rate of 60 mL/min over 12 min). The desired diastereomer was collected at a retention time of 10.2 min. LCMS calculated for C₂₉H₂₄D₄F₃N₁₀O₃ (M+H)⁺: m/z=625.2.; found: 625.3.

### Example 114. Methyl ((1R,3R)-3-(6-((1-((1-cyanocyclopropyl)methyl)-3-(1-(difluoromethyl)-1H-pyrazol-4-yl)-2-oxo-2,3-dihydro-1H-imidazo[4,5-b]pyridin-5-yl)amino)-3-(methyl-d₃)-2-oxo-2,3-dihydro-1H-imidazo[4,5-c]pyridin-1-yl)cyclopentyl)carbamate

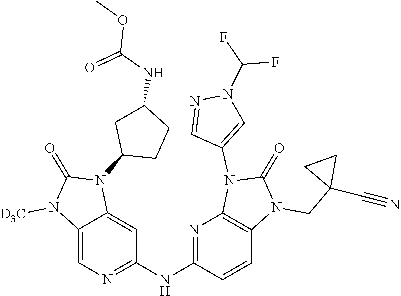 [SMILES: [2H]C([2H])([2H])n1c(=O)n([C@@H]2CC[C@@H](NC(=O)OC)C2)c2cc(Nc3ccc4c(n3)n(-c3cnn(C(F)F)c3)c(=O)n4CC3(C#N)CC3)ncc21]

### Step 1. 6-Chloro-N-(1-(difluoromethyl)-1H-pyrazol-4-yl)-3-nitropyridin-2-amine

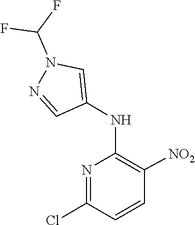 [SMILES: O=[N+]([O-])c1ccc(Cl)nc1Nc1cnn(C(F)F)c1]

To a solution of 2,6-dichloro-3-nitropyridine (0.700 g, 3.63 mmol) and 1-(difluoromethyl)-1H-pyrazol-4-amine (0.579 g, 4.35 mmol) in ethanol (12.1 mL) was added sodium bicarbonate (0.4 g, 4.7 mmol). After stirring at r.t. for 64 h, the reaction mixture was diluted with water (40 mL) and extracted into 150 mL of ethyl acetate. The ethyl acetate layer was washed with brine, dried over anhydrous sodium sulfate, filtered, and concentrated in vacuo to afford an orange solid. The crude material was suspended in dichloromethane and was filtered. The filtrate was purified by Biotage Isolera and combined with the previously isolated solid to give the product as an orange solid. LCMS calculated for C₉H₇ClF₂N₅O₂ (M+H)⁺: m/z=290.0; found: 290.1.

### Step 2. N²-(1-(Difluoromethyl)-1H-pyrazol-4-yl)-N⁶-(2,4-dimethoxybenzyl)-3-nitropyridine-2,6-diamine

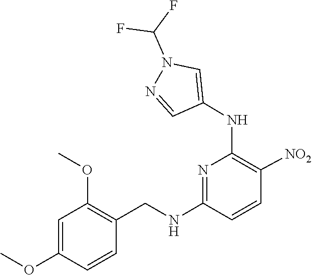 [SMILES: COc1ccc(CNc2ccc([N+](=O)[O-])c(Nc3cnn(C(F)F)c3)n2)c(OC)c1]

To a solution of 6-chloro-N-(1-(difluoromethyl)-1H-pyrazol-4-yl)-3-nitropyridin-2-amine (0.70 g, 2.42 mmol and 1-(2,4-dimethoxyphenyl)methanamine (1.1 mL, 7.3 mmol) in ethanol (12 mL) was added potassium carbonate (0.50 g, 3.6 mmol). After stirring at 50° C. for 14 h, the reaction mixture was diluted with water (100 mL) and extracted into 100 mL of ethyl acetate. The ethyl acetate layer was washed with brine, dried over anhydrous sodium sulfate, filtered, and concentrated in vacuo to afford brown solid. The crude material was suspended in dichloromethane and was filtered. The filtrate was purified by Biotage Isolera and combined with the previously isolated solid to give the product as a yellow solid. LCMS calculated for C₁₈H₁₉ClF₂N₆O₄ (M+H)⁺: m/z=421.1; found: 421.2.

### Step 3. N²-(1-(Difluoromethyl)-1H-pyrazol-4-yl)-N⁶-(2,4-dimethoxybenzyl)pyridine-2,3,6-triamine

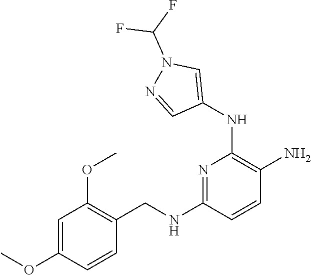 [SMILES: COc1ccc(CNc2ccc(N)c(Nc3cnn(C(F)F)c3)n2)c(OC)c1]

To a suspension of N²-(1-(difluoromethyl)-1H-pyrazol-4-yl)-N⁶-(2,4-dimethoxybenzyl)-3-nitropyridine-2,6-diamine (0.50 g, 1.19 mmol) and ammonium chloride (0.44 g, 8.3 mmol) in methanol (12 mL) was added zinc (0.43 g, 6.5 mmol) in two portions over 5 minutes. After stirring for 1.5 h, the reaction mixture was diluted with ethyl acetate (50 ml) and was filtered through a 0.45 micron filter cartridge. The filtrate was washed with saturated sodium bicarbonate solution (40 ml), brine (40 ml), dried over anhydrous sodium sulfate, filtered, and concentrated in vacuo give the desired product as a blue solid. The resulting crude material was used in next step without further purification. LCMS calculated for C₁₈H₂₁F₂N₆O₂ (M+H)⁺: m/z=391.2; found: 391.2.

### Step 4. 3-(1-(Difluoromethyl)-1H-pyrazol-4-yl)-5-((2,4-dimethoxybenzyl)amino)-1,3-dihydro-2H-imidazo[4,5-b]pyridin-2-one

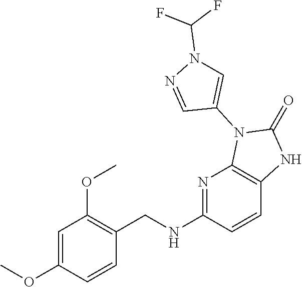 [SMILES: COc1ccc(CNc2ccc3[nH]c(=O)n(-c4cnn(C(F)F)c4)c3n2)c(OC)c1]

This compound was prepared according to procedure Example 89, step 4 with N²-(1-(difluoromethyl)-1H-pyrazol-4-yl)-N⁶-(2,4-dimethoxybenzyl)pyridine-2,3,6-triamine replacing tert-butyl (3-amino-6-chloro-2-((1-(difluoromethyl)-1H-pyrazol-4-yl)amino)pyridin-4-yl)carbamate. LCMS calculated for C₁₉H₁₉F₂N₆O₃ (M+H)⁺: m/z=417.1; found: 417.2.

### Step 5. 1-((3-(1-(Difluoromethyl)-1H-pyrazol-4-yl)-5-((2,4-dimethoxybenzyl)amino)-2-oxo-2,3-dihydro-1H-imidazo[4,5-b]pyridin-1-yl)methyl)cyclopropane-1-carbonitrile

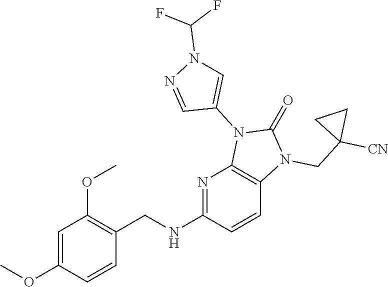 [SMILES: COc1ccc(CNc2ccc3c(n2)n(-c2cnn(C(F)F)c2)c(=O)n3CC2(C#N)CC2)c(OC)c1]

To a solution of 3-(1-(difluoromethyl)-1H-pyrazol-4-yl)-5-((2,4-dimethoxybenzyl)amino)-1,3-dihydro-2H-imidazo[4,5-b]pyridin-2-one in DMF (1.2 mL) was added cesium carbonate (0.14 g, 0.42 mmol) followed by dropwise addition of a solution of 1-(bromomethyl)cyclopropane-1-carbonitrile (58 mg, 0.36 mmol) in DMF (0.2 mL). The mixture was stirred for 60 min at 60° C. The reaction mixture was diluted with water (15 mL) and was extracted into ethyl acetate (30 mL). The EtOAc extract was washed with brine, dried over anhydrous sodium sulfate, filtered, and concentrated in-vacuo to rust colored solid. The crude solid was further purified by Biotage Isolera and combined to give the product as a white solid. LCMS calculated for C₂₄H₂₄F₂N₇O₃ (M+H)⁺: m/z=496.2; found: 496.2.

### Step 6. 1-((5-Amino-3-(1-(difluoromethyl)-1H-pyrazol-4-yl)-2-oxo-2,3-dihydro-1H-imidazo[4,5-b]pyridin-1-yl)methyl)cyclopropane-1-carbonitrile

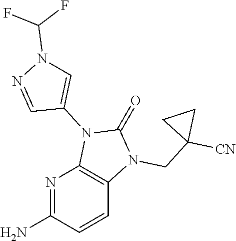 [SMILES: N#CC1(Cn2c(=O)n(-c3cnn(C(F)F)c3)c3nc(N)ccc32)CC1]

To a vial containing 1-((3-(1-(difluoromethyl)-1H-pyrazol-4-yl)-5-((2,4-dimethoxybenzyl)amino)-2-oxo-2,3-dihydro-1H-imidazo[4,5-b]pyridin-1-yl)methyl)cyclopropane-1-carbonitrile (50 mg, 0.10 mmol) was added TFA (1.4 mL). After stirring at r.t. for 30 min, the mixture was concentrated in vacuo to residue. Acetonitrile (2.5 mL) was added and the resulting suspension was concentrated. This step was repeated 3 times to remove excess of TFA. At last, the remaining residue was suspended in acetonitrile (3 mL) and filtered through a 0.45 micron filter cartridge. The filtrate was concentrated in vacuo to afford a yellow residue. The resulting crude material was used in next step without further purification. LCMS calculated for C₁₅H₁₄F₂N₇O (M+H)⁺: m/z=346.1; found: 346.2.

### Step 7. Methyl ((1R,3R)-3-(6-((1-((1-cyanocyclopropyl)methyl)-3-(1-(difluoromethyl)-1H-pyrazol-4-yl)-2-oxo-2,3-dihydro-1H-imidazo[4,5-b]pyridin-5-yl)amino)-3-(methyl-d₃)-2-oxo-2,3-dihydro-1H-imidazo[4,5-c]pyridin-1-yl)cyclopentyl)carbamate

This compound was prepared according to procedures described in Example 92, step 6 with 1-((5-amino-3-(1-(difluoromethyl)-1H-pyrazol-4-yl)-2-oxo-2,3-dihydro-1H-imidazo[4,5-b]pyridin-1-yl)methyl)cyclopropane-1-carbonitrile replacing 2-(2-amino-6-(5-chlorothiazol-2-yl)pyridin-4-yl)propan-2-ol. LCMS calculated for C₂₉H₂₇D₃F₂N₁₁O₄ (M+H)⁺: m/z=637.3; found: 637.3.

### Example 115. Methyl ((1R,3R)-3-(6-((3-(1-(difluoromethyl)-1H-pyrazol-4-yl)-7-(2-hydroxypropan-2-yl)-2-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl)carbamate

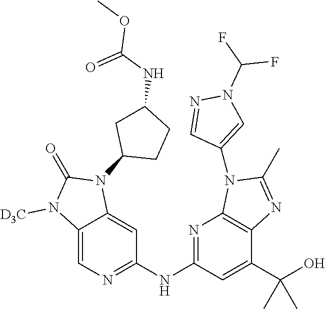 [SMILES: [2H]C([2H])([2H])n1c(=O)n([C@@H]2CC[C@@H](NC(=O)OC)C2)c2cc(Nc3cc(C(C)(C)O)c4nc(C)n(-c5cnn(C(F)F)c5)c4n3)ncc21]

### Step 1. tert-Butyl (5-chloro-3-(1-(difluoromethyl)-1H-pyrazol-4-yl)-2-methyl-3H-imidazo[4,5-b]pyridin-7-yl)carbamate

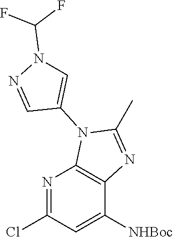 [SMILES: Cc1nc2c(NC(=O)OC(C)(C)C)cc(Cl)nc2n1-c1cnn(C(F)F)c1]

To a solution of tert-butyl (3-amino-6-chloro-2-((1-(difluoromethyl)-1H-pyrazol-4-yl)amino)pyridin-4-yl)carbamate (640 mg, 1.7 mmol) (Example 89, step 3) in acetic acid (11.4 mL) was added triethyl orthoacetate (0.8 mL, 4.3 mmol). The reaction mixture was stirred for 1.5 h at 65° C. The reaction mixture was concentrated in-vacuo. The solid was suspended in saturated sodium bicarbonate solution (20 mL) and was extracted into ethyl acetate (70 mL). The EtOAc extract was washed with brine, dried over anhydrous sodium sulfate, filtered, and concentrated in-vacuo to purple solid. The crude solid was further purified by Biotage Isolera to give the product as a pink solid. LCMS calculated for C₁₆H₁₈ClF₂N₆O₂ (M+H)⁺: m/z=399.1; found: 399.1.

### Step 2. 5-Chloro-3-(1-(difluoromethyl)-1H-pyrazol-4-yl)-2-methyl-3H-imidazo[4,5-b]pyridin-7-amine

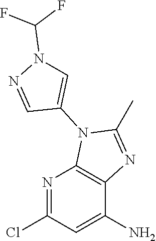 [SMILES: Cc1nc2c(N)cc(Cl)nc2n1-c1cnn(C(F)F)c1]

This compound was prepared according to procedure described in Example 89, step 5 with tert-butyl (5-chloro-3-(1-(difluoromethyl)-1H-pyrazol-4-yl)-2-methyl-3H-imidazo[4,5-b]pyridin-7-yl)carbamate replacing of tert-butyl (5-chloro-3-(1-(difluoromethyl)-1H-pyrazol-4-yl)-2-oxo-2,3-dihydro-1H-imidazo[4,5-b]pyridin-7-yl)carbamate. LCMS calculated for C₁₁H₁₀ClF₂N₆ (M+H)⁺: m/z=299.1; found: 299.1.

### Step 3. 7-Bromo-5-chloro-3-(1-(difluoromethyl)-1H-pyrazol-4-yl)-2-methyl-3H-imidazo[4,5-b]pyridine

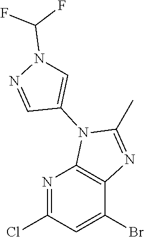 [SMILES: Cc1nc2c(Br)cc(Cl)nc2n1-c1cnn(C(F)F)c1]

This compound was prepared according to procedure described in Example 89, step 7 with 5-chloro-3-(1-(difluoromethyl)-1H-pyrazol-4-yl)-2-methyl-3H-imidazo[4,5-b]pyridin-7-amine replacing 7-amino-5-chloro-3-(1-(difluoromethyl)-1H-pyrazol-4-yl)-1-methyl-1,3-dihydro-2H-imidazo[4,5-b]pyridin-2-one. LCMS calculated for C₁₁H₈BrClF₂N₅ (M+H)⁺: m/z=362.0, 364.0; found: 362.0, 364.0.

### Step 4. 5-Chloro-3-(1-(difluoromethyl)-1H-pyrazol-4-yl)-2-methyl-7-(prop-1-en-2-yl)-3H-imidazo[4,5-b]pyridine

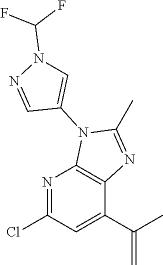 [SMILES: C=C(C)c1cc(Cl)nc2c1nc(C)n2-c1cnn(C(F)F)c1]

This compound was prepared according to procedure described in Example 89, step 8 with 7-bromo-5-chloro-3-(1-(difluoromethyl)-1H-pyrazol-4-yl)-2-methyl-3H-imidazo[4,5-b]pyridine replacing 7-bromo-5-chloro-3-(1-(difluoromethyl)-1H-pyrazol-4-yl)-1-methyl-1,3-dihydro-2H-imidazo[4,5-b]pyridin-2-one and 4,4,5,5-tetramethyl-2-(prop-1-en-2-yl)-1,3,2-dioxaborolane (0.102 ml, 0.552 mmol) in place of 3-(4,4,5,5-tetramethyl-1,3,2-dioxaborolan-2-yl)-4-(trifluoromethyl)pyridine. LCMS calculated for C₁₄H₁₃ClF₂N₅ (M+H)⁺: m/z=324.1; found: 324.1.

### Step 5. 2-(5-Chloro-3-(1-(difluoromethyl)-1H-pyrazol-4-yl)-2-methyl-3H-imidazo[4,5-b]pyridin-7-yl)propan-2-ol

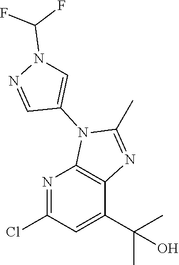 [SMILES: Cc1nc2c(C(C)(C)O)cc(Cl)nc2n1-c1cnn(C(F)F)c1]

To a mixture of 5-chloro-3-(1-(difluoromethyl)-1H-pyrazol-4-yl)-2-methyl-7-(prop-1-en-2-yl)-3H-imidazo[4,5-b]pyridine (109 mg, 0.34 mmol) in anhydrous MeOH (8.4 ml) was added sodium bicarbonate (57 mg, 0.67 mmol), methyl 4-nitrobenzenesulfonate (110 mg, 0.51 mmol) and iron(III) acetylacetonate (12 mg, 0.034 mmol). The mixture was bubbled with oxygen for 10 minutes and cooled to 0° C. while being kept under an oxygen atmosphere. Phenylsilane (0.12 mL, 1.0 mmol) was added dropwise before the ice cooling bath was removed and stirred for 30 minutes. The reaction mixture was diluted with acetonitrile and was filtered through a 0.45 micron filter cartridge. The filtrate was purified with prep-LCMS (XBridge C18 column, eluting with a gradient of acetonitrile/water containing 0.4% NH₄OH, at flow rate of 60 mL/min). LCMS calculated for C₁₄H₁₅ClF₂N₅O (M+H)⁺: m/z=342.1; found: 342.1.

### Step 6. Methyl (1R,3R)-3-(6-((3-(1-(difluoromethyl)-1H-pyrazol-4-yl)-7-(2-hydroxypropan-2-yl)-2-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl)carbamate

This compound was prepared according to procedure described in Example 89, step 9 using 2-(5-chloro-3-(1-(difluoromethyl)-1H-pyrazol-4-yl)-2-methyl-3H-imidazo[4,5-b]pyridin-7-yl)propan-2-ol in place of 5-chloro-3-(1-(difluoromethyl)-1H-pyrazol-4-yl)-1-methyl-7-(4-(trifluoromethyl)pyridin-3-yl)-1,3-dihydro-2H-imidazo[4,5-b]pyridin-2-one. LCMS calculated for C₂₈H₃₀D₃F₂N₁₀O₄(M+H)⁺: m/z=614.3; found: 614.3. ¹H NMR (500 MHz, DMSO-d₆) δ: 9.45 (s, 1H), 8.81 (s, 1H), 8.35 (s, 1H), 7.99 (s, 1H), 7.93 (t, ²J_{H-F}=58.6 Hz, 1H), 7.65 (s, 1H), 7.60 (s, 1H), 7.26 (d, J=7.2 Hz, 1H), 5.26 (s, 1H), 4.71 (p, J=8.3 Hz, 1H), 4.21 (m, 1H), 3.53 (s, 3H), 2.49 (s, 3H), 2.20 (m, 1H), 2.14-2.06 (m, 1H), 2.08-1.99 (m, 1H), 1.98-1.88 (m, 1H), 1.90-1.79 (m, 1H), 1.70 (s, 6H), 1.65-1.55 (m, 1H).

### Example 116. N-((1R,3R)-1-methyl-3-(3-(methyl-d₃)-6-((4-(morpholine-4-carbonyl)-8-(trifluoromethyl)quinolin-2-yl)amino)-2-oxo-2,3-dihydro-1H-imidazo[4,5-c]pyridin-1-yl)cyclopentyl)cyclopropanecarboxamide

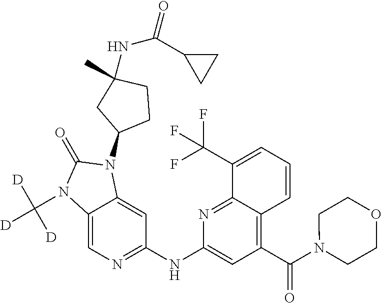 [SMILES: [2H]C([2H])([2H])n1c(=O)n([C@@H]2CC[C@@](C)(NC(=O)C3CC3)C2)c2cc(Nc3cc(C(=O)N4CCOCC4)c4cccc(C(F)(F)F)c4n3)ncc21]

### Step 1. N-((1R,3R)-3-(6-Chloro-3-(methyl-d₃)-2-oxo-2,3-dihydro-1H-imidazo[4,5-c]pyridin-1-yl)-1-methylcyclopentyl)cyclopropanecarboxamide

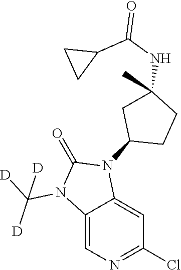 [SMILES: [2H]C([2H])([2H])n1c(=O)n([C@@H]2CC[C@@](C)(NC(=O)C3CC3)C2)c2cc(Cl)ncc21]

This compound was prepared according to the procedures described in Example 92, with tert-butyl ((1R,3R)-3-amino-1-methylcyclopentyl)carbamate replacing with tert-butyl ((1S,3R)-3-amino-1-methylcyclopentyl)carbamate in Step 1. LCMS calculated for C₁₇H₁₉D₃ClN₄O₂ (M+H)⁺: m/z=352.2; found: 352.2.

### Step 2. N-((1R,3R)-1-methyl-3-(3-(methyl-d₃)-6-((4-(morpholine-4-carbonyl)-8-(trifluoromethyl)quinolin-2-yl)amino)-2-oxo-2,3-dihydro-1H-imidazo[4,5-c]pyridin-1-yl)cyclopentyl)cyclopropanecarboxamide

This compound was prepared according to the procedures described in Example 52, with N-((1R,3R)-3-(6-amino-3-(methyl-d₃)-2-oxo-2,3-dihydro-1H-imidazo[4,5-c]pyridin-1-yl)-1-methylcyclopentyl)cyclopropanecarboxamide replacing methyl ((1R,3R)-3-(6-amino-3-(methyl-d₃)-2-oxo-2,3-dihydro-1H-imidazo[4,5-c]pyridin-1-yl)cyclopentyl)carbamate in Step 5. LCMS calculated for C₃₂H₃₂D₃F₃N₇O₄ (M+H)⁺: m/z=641.3; found: 641.3.

### Example 117. N-((1S,3R)-3-(6-((7-(hydroxymethyl)-2-methyl-3-(1-(trifluoromethyl)-1H-pyrazol-4-yl)-2H-pyrazolo[4,3-b]pyridin-5-yl)amino)-3-(methyl-d₃)-2-oxo-2,3-dihydro-1H-imidazo[4,5-c]pyridin-1-yl)-1-methylcyclopentyl)cyclopropanecarboxamide

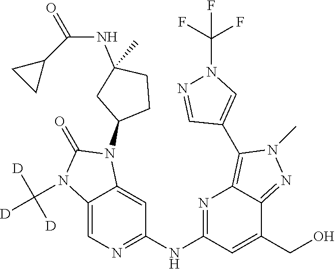 [SMILES: [2H]C([2H])([2H])n1c(=O)n([C@@H]2CC[C@](C)(NC(=O)C3CC3)C2)c2cc(Nc3cc(CO)c4nn(C)c(-c5cnn(C(F)(F)F)c5)c4n3)ncc21]

### Step 1. 3-Bromo-7-(methoxycarbonyl)-2-methyl-2H-pyrazolo[4,3-b]pyridine 4-oxide

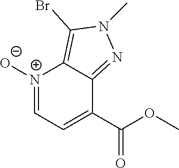 [SMILES: COC(=O)c1cc[n+]([O-])c2c(Br)n(C)nc12]

To a vial containing methyl 3-bromo-2-methyl-2H-pyrazolo[4,3-b]pyridine-7-carboxylate (0.1 g, 0.37 mmol) in DCM (2.0 mL) was added 3-chloroperbenzoic acid (0.167 g, 0.74 mmol, 77%). The reaction was stirred at r.t. overnight. The reaction mixture was quenched with sat. Na₂S₂O₃ solution and extracted with DCM. The organic layers were combined, washed with sat. NaHCO₃ solution, dried over sodium sulfate, filtered, and the solvents were evaporated in vacuo. The crude material was used in the next step without further purification. LCMS calculated for C₉H₉BrN₃O₃ (M+H)⁺: m/z=286.0; found: 285.9.

### Step 2. 7-(Methoxycarbonyl)-2-methyl-3-(1-(trifluoromethyl)-1H-pyrazol-4-yl)-2H-pyrazolo[4,3-b]pyridine 4-oxide

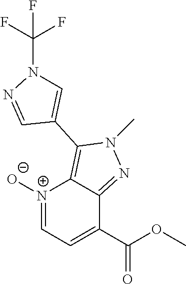 [SMILES: COC(=O)c1cc[n+]([O-])c2c(-c3cnn(C(F)(F)F)c3)n(C)nc12]

To a vial containing 3-bromo-7-(methoxycarbonyl)-2-methyl-2H-pyrazolo[4,3-b]pyridine 4-oxide (14 mg, 0.05 mmol), 4-(4,4,5,5-tetramethyl-1,3,2-dioxaborolan-2-yl)-1-(trifluoromethyl)-1H-pyrazole (39 mg, 0.15 mmol), sodium carbonate (11 mg, 0.1 mmol) and XPhos Pd G2 (39 mg, 0.05 mmol) was added dioxane (0.6 mL) and water (0.12 mL) under N₂ atmosphere. The reaction was stirred at 60° C. overnight. The mixture was cooled down to r.t. and diluted with DCM. The organic layer was washed with sat. NaHCO₃ solution, dried over sodium sulfate, filtered, and the solvents were evaporated in vacuo. The crude material was used in the next step without further purification. LCMS calculated for C₁₃H₁₁F₃N₅O₃ (M+H)⁺: m/z=342.1; found: 342.0.

### Step 3. Methyl 5-chloro-2-methyl-3-(1-(trifluoromethyl)-1H-pyrazol-4-yl)-2H-pyrazolo[4,3-b]pyridine-7-carboxylate

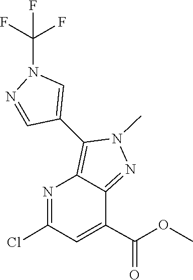 [SMILES: COC(=O)c1cc(Cl)nc2c(-c3cnn(C(F)(F)F)c3)n(C)nc12]

To a vial containing 7-(methoxycarbonyl)-2-methyl-3-(1-(trifluoromethyl)-1H-pyrazol-4-yl)-2H-pyrazolo[4,3-b]pyridine 4-oxide (100 mg, 0.29 mmol) was added phosphorus(V) oxychloride (0.8 mL). The reaction was stirred at 70° C. for 2 h. The mixture was cooled down to r.t. and concentrated in vacuo. The resulting residue was diluted with DCM, washed with sat. NaHCO₃ solution, dried over sodium sulfate, filtered, and the solvents were evaporated in vacuo. The crude material was purified by Biotage Isolera to give the product. LCMS calculated for C₁₃H₁₀ClF₃N₅O₂ (M+H)⁺: m/z=360.0; found: 360.0.

### Step 4. (5-Chloro-2-methyl-3-(1-(trifluoromethyl)-1H-pyrazol-4-yl)-2H-pyrazolo[4,3-b]pyridin-7-yl) methanol

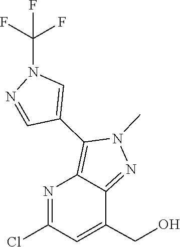 [SMILES: Cn1nc2c(CO)cc(Cl)nc2c1-c1cnn(C(F)(F)F)c1]

This compound was prepared according to the procedures described in Example 63, Step 3. LCMS calculated for C₁₂H₁₀ClF₃N₅O (M+H)⁺: m/z=332.0; found: 332.0.

### Step 5. N-((1S,3R)-3-(6-((7-(hydroxymethyl)-2-methyl-3-(1-(trifluoromethyl)-1H-pyrazol-4-yl)-2H-pyrazolo[4,3-b]pyridin-5-yl)amino)-3-(methyl-d₃)-2-oxo-2,3-dihydro-1H-imidazo[4,5-c]pyridin-1-yl)-1-methylcyclopentyl)cyclopropanecarboxamide

This compound was prepared according to the procedures described in Example 91, with methyl ((1S,3R)-3-(6-amino-3-(methyl-d₃)-2-oxo-2,3-dihydro-1H-imidazo[4,5-c]pyridin-1-yl)-1-methylcyclopentyl)carbamate replacing N-((1s,4s)-4-(6-amino-3-(methyl-d₃)-2-oxo-2,3-dihydro-1H-imidazo[4,5-c]pyridin-1-yl)-1-methylcyclohexyl)cyclopropanecarboxamide and (5-chloro-2-methyl-3-(1-(trifluoromethyl)-1H-pyrazol-4-yl)-2H-pyrazolo[4,3-b]pyridin-7-yl)methanol replacing 5-chloro-2-methyl-3-(1-(trifluoromethyl)-1H-pyrazol-4-yl)-2H-pyrazolo[4,3-b]pyridine in Step 6. LCMS calculated for C₂₉H₂₉D₃F₃N₁₀O₃ (M+H)⁺: m/z=628.3; found: 628.3.

### Example 118. Methyl ((1R,3R)-3-(6-((1-(2-hydroxy-2-methylpropyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)cyclopentyl)carbamate

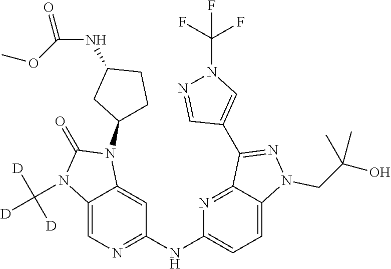 [SMILES: [2H]C([2H])([2H])n1c(=O)n([C@@H]2CC[C@@H](NC(=O)OC)C2)c2cc(Nc3ccc4c(n3)c(-c3cnn(C(F)(F)F)c3)nn4CC(C)(C)O)ncc21]

### Step 1.1-(5-Chloro-3-iodo-1H-pyrazolo[4,3-b]pyridin-1-yl)-2-methylpropan-2-ol

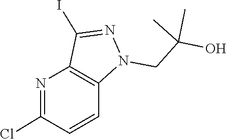 [SMILES: CC(C)(O)Cn1nc(I)c2nc(Cl)ccc21]

To a vial containing 5-chloro-3-iodo-1H-pyrazolo[4,3-b]pyridine (0.39 g, 1.4 mmol), 2,2-dimethyloxirane (0.10 g, 1.4 mmol) in DMF (4.0 mL) was added potassium carbonate (0.58 g, 4.2 mmol). The reaction was stirred at 100° C. for 2 h. The mixture was cooled down to r.t., the solids were filtered off, and the solvent was evaporated in vacuo. The crude material was purified by Biotage Isolera to give the product as first peak. LCMS calculated for C₁₀H₁₂ClIN₃O (M+H)⁺: m/z=352.0; found 351.9.

### Step 2.1-(5-Chloro-3-(1-(trifluoromethyl)-1H-pyrazol-4-yl)-1H-pyrazolo[4,3-b]pyridin-1-yl)-2-methylpropan-2-ol

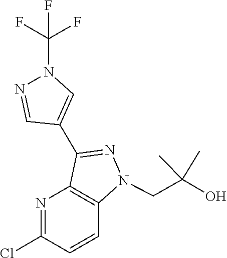 [SMILES: CC(C)(O)Cn1nc(-c2cnn(C(F)(F)F)c2)c2nc(Cl)ccc21]

This compound was prepared according to the procedures described in Example 117, with 1-(5-chloro-3-iodo-1H-pyrazolo[4,3-b]pyridin-1-yl)-2-methylpropan-2-ol replacing 3-bromo-7-(methoxycarbonyl)-2-methyl-2H-pyrazolo[4,3-b]pyridine 4-oxide in Step 2. LCMS calculated for C₁₄H₁₄ClF₃N₅O (M+H)⁺: m/z=360.1; found: 360.1.

### Step 3. Methyl ((1R,3R)-3-(6-((1-(2-hydroxy-2-methylpropyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)cyclopentyl)carbamate

This compound was prepared according to the procedures described in Example 91, with 1-(5-chloro-3-(1-(trifluoromethyl)-1H-pyrazol-4-yl)-1H-pyrazolo[4,3-b]pyridin-1-yl)-2-methylpropan-2-ol replacing 5-chloro-2-methyl-3-(1-(trifluoromethyl)-1H-pyrazol-4-yl)-2H-pyrazolo[4,3-b]pyridine and methyl ((1R,3R)-3-(6-amino-3-(methyl-d₃)-2-oxo-2,3-dihydro-1H-imidazo[4,5-c]pyridin-1-yl)cyclopentyl)carbamate replacing N-((1s,4s)-4-(6-amino-3-(methyl-d₃)-2-oxo-2,3-dihydro-1H-imidazo[4,5-c]pyridin-1-yl)-1-methylcyclohexyl)cyclopropane carboxamide in Step 6. LCMS calculated for C₂₈H₂₉D₃F₃N₁₀O₄ (M+H)⁺: m/z=632.3; found: 632.3. ¹H NMR (500 MHz, DMSO) δ 10.94 (s, 1H), 8.97 (s, 1H), 8.62 (s, 1H), 8.28 (d, J=9.1 Hz, 1H), 8.18 (s, 1H), 7.40 (s, 1H), 7.34-7.29 (m, 2H), 4.94 (p, J=8.5 Hz, 1H), 4.38 (s, 2H), 4.18 (d, J=7.8 Hz, 1H), 3.53 (s, 3H), 2.29 (m, 1H), 2.18-2.09 (m, 2H), 2.08-2.00 (m, 1H), 1.92 (m, 1H), 1.60 (q, J=7.9 Hz, 1H), 1.17 (s, 6H).

### Example 119. N-((1S,3R)-3-(6-((1-(2-Hydroxy-2-methylpropyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)-1-methylcyclopentyl)cyclopropanecarboxamide

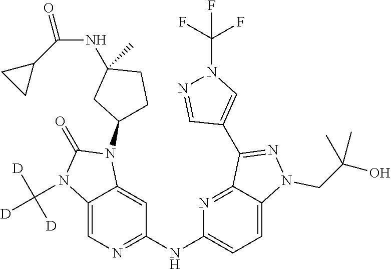 [SMILES: [2H]C([2H])([2H])n1c(=O)n([C@@H]2CC[C@](C)(NC(=O)C3CC3)C2)c2cc(Nc3ccc4c(n3)c(-c3cnn(C(F)(F)F)c3)nn4CC(C)(C)O)ncc21]

This compound was prepared according to the procedures described in Example 117, with 1-(5-chloro-3-(1-(trifluoromethyl)-1H-pyrazol-4-yl)-1H-pyrazolo[4,3-b]pyridin-1-yl)-2-methylpropan-2-ol replacing (5-chloro-2-methyl-3-(1-(trifluoromethyl)-1H-pyrazol-4-yl)-2H-pyrazolo[4,3-b]pyridin-7-yl)methanol in Step 5. LCMS calculated for C₃₁H₃₃D₃F₃N₁₀O₃ (M+H)⁺: m/z=656.3; found: 656.3. ¹H NMR (500 MHz, DMSO) δ 10.98 (s, 1H), 8.96 (s, 1H), 8.64 (s, 1H), 8.28 (d, J=9.1 Hz, 1H), 8.21 (s, 1H), 8.13 (s, 1H), 7.60 (s, 1H), 7.29 (d, J=9.1 Hz, 1H), 4.82 (m, 1H), 4.37 (s, 2H), 2.44 (dd, J=13.0, 9.9 Hz, 1H), 2.32-2.21 (m, 2H), 2.17-2.07 (m, 2H), 1.85-1.75 (m, 1H), 1.54 (h, J=5.7 Hz, 1H), 1.35 (s, 3H), 1.17 (s, 6H), 0.62-0.52 (m, 4H).

### Example 120. N-((1s,4s)-4-(6-((1-(2-Hydroxy-2-methylpropyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)-1-methylcyclohexyl)cyclopropanecarboxamide

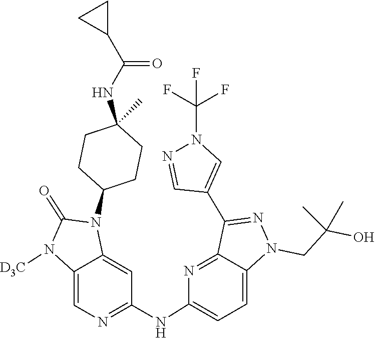 [SMILES: [2H]C([2H])([2H])n1c(=O)n([C@H]2CC[C@@](C)(NC(=O)C3CC3)CC2)c2cc(Nc3ccc4c(n3)c(-c3cnn(C(F)(F)F)c3)nn4CC(C)(C)O)ncc21]

This compound was prepared according to the procedures described in Example 91, with 1-(5-chloro-3-(1-(trifluoromethyl)-1H-pyrazol-4-yl)-1H-pyrazolo[4,3-b]pyridin-1-yl)-2-methylpropan-2-ol replacing 5-chloro-2-methyl-3-(1-(trifluoromethyl)-1H-pyrazol-4-yl)-2H-pyrazolo[4,3-b]pyridine in Step 6. LCMS calculated for C₃₂H₃₅D₃F₃N₁₀O₃ (M+H)⁺: m/z=670.3; found: 670.3. ¹H NMR (500 MHz, DMSO) δ 10.72 (s, 1H), 8.93 (s, 1H), 8.66 (s, 1H), 8.23 (d, J=9.2 Hz, 1H), 8.18 (s, 1H), 7.68 (s, 1H), 7.48 (s, 1H), 7.27 (d, J=9.1 Hz, 1H), 4.36 (s, 2H), 4.08 (m, 1H), 2.50-2.32 (m, 4H), 1.73-1.63 (m, 1H), 1.65-1.58 (m, 2H), 1.40 (m, 2H), 1.25 (s, 3H), 1.16 (s, 6H), 0.65-0.52 (m, 4H).

### Example 121. N-((1S,3R)-3-(6-((2-((1s,3s)-3-Hydroxycyclobutyl)-3-(1-(trifluoromethyl)-1H-pyrazol-4-yl)-2H-pyrazolo[4,3-b]pyridin-5-yl)amino)-3-(methyl-d₃)-2-oxo-2,3-dihydro-1H-imidazo[4,5-c]pyridin-1-yl)-1-methylcyclopentyl)cyclopropanecarboxamide

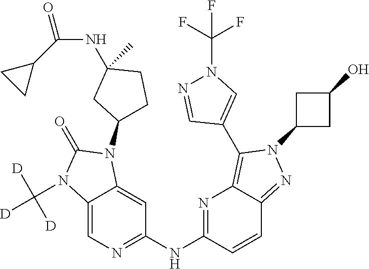 [SMILES: [2H]C([2H])([2H])n1c(=O)n([C@@H]2CC[C@](C)(NC(=O)C3CC3)C2)c2cc(Nc3ccc4nn([C@H]5C[C@@H](O)C5)c(-c5cnn(C(F)(F)F)c5)c4n3)ncc21]

### Step 1. 2-((1s,3s)-3-((tert-Butyldimethylsilyl)oxy)cyclobutyl)-5-chloro-3-iodo-2H-pyrazolo[4,3-b]pyridine

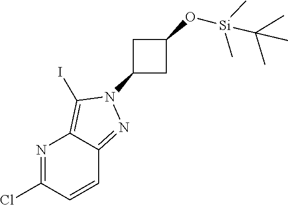 [SMILES: CC(C)(C)[Si](C)(C)O[C@H]1C[C@@H](n2nc3ccc(Cl)nc3c2I)C1]

To a cooled vial containing 5-chloro-3-iodo-1H-pyrazolo[4,3-b]pyridine (0.39 g, 1.4 mmol), (1r,3r)-3-((tert-butyldimethylsilyl)oxy)cyclobutan-1-ol (0.43 g, 2.1 mmol) and triphenylphosphine (0.55 g, 2.1 mmol) in THF (5.0 mL) was added DIAD (0.43 g, 2.1 mmol). The mixture was warmed to r.t. and stirred for 2 h. The solvents were evaporated in vacuo and the crude material was purified by Biotage Isolera to give the product. LCMS calculated for C₁₆H₂₄ClIN₃OSi (M+H)⁺: m/z=464.0; found: 464.1.

### Step 2. (1s, 3s)-3-(5-Chloro-3-(1-(trifluoromethyl)-1H-pyrazol-4-yl)-2H-pyrazolo[4,3-b]pyridin-2-yl)cyclobutan-1-ol

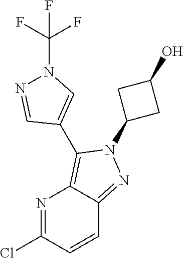 [SMILES: O[C@H]1C[C@@H](n2nc3ccc(Cl)nc3c2-c2cnn(C(F)(F)F)c2)C1]

To a vial containing 2-((1s,3s)-3-((tert-butyldimethylsilyl)oxy)cyclobutyl)-5-chloro-3-iodo-2H-pyrazolo[4,3-b]pyridine (0.14 g, 0.3 mmol), 4-(4,4,5,5-tetramethyl-1,3,2-dioxaborolan-2-yl)-1-(trifluoromethyl)-1H-pyrazole (0.16 g, 0.6 mmol), tripotassium phosphate (0.26 g, 1.2 mmol) and Pd(dppf)₂Cl₂ dichloromethane adduct (0.022 g, 0.03 mmol) was added dioxane (2.0 mL) and water (0.8 mL) under N₂ atmosphere. The reaction was stirred at 60° C. for 1 h. The mixture was cooled down to r.t., the solids were filtered off, and the solvent was evaporated in vacuo. The crude material was purified by Biotage Isolera. The resulting mixture was treated with MeCN (2 mL) and IN HCl (2 mL) and stirred at r.t. for 1 h. The solvents were evaporated in vacuo and the crude material was used in the next step without further purification. LCMS calculated for C₁₄H₁₂ClF₃N₅O (M+H)⁺: m/z=358.1; found 358.1.

### Step 3. N-((1S,3R)-3-(6-((2-((1s,3S)-3-Hydroxycyclobutyl)-3-(1-(trifluoromethyl)-1H-pyrazol-4-yl)-2H-pyrazolo[4,3-b]pyridin-5-yl)amino)-3-(methyl-d₃)-2-oxo-2,3-dihydro-1H-imidazo[4,5-c]pyridin-1-yl)-1-methylcyclopentyl) cyclopropanecarboxamide

This compound was prepared according to the procedures described in Example 117, with (1s,3s)-3-(5-chloro-3-(1-(trifluoromethyl)-1H-pyrazol-4-yl)-2H-pyrazolo[4,3-b]pyridin-2-yl)cyclobutan-1-ol replacing (5-chloro-2-methyl-3-(1-(trifluoromethyl)-1H-pyrazol-4-yl)-2H-pyrazolo[4,3-b]pyridin-7-yl)methanol in Step 5. LCMS calculated for C₃₁H₃₁D₃F₃N₁₀O₃ (M+H)⁺: m/z=654.3; found: 654.3.

### Example 122. Methyl ((1R,3R)-3-(6-((2-(2-hydroxy-2-methylpropyl)-3-(1-(trifluoromethyl)-1H-pyrazol-4-yl)-2H-pyrazolo[4,3-b]pyridin-5-yl)amino)-3-(methyl-d₃)-2-oxo-2,3-dihydro-1H-imidazo[4,5-c]pyridin-1-yl)cyclopentyl)carbamate

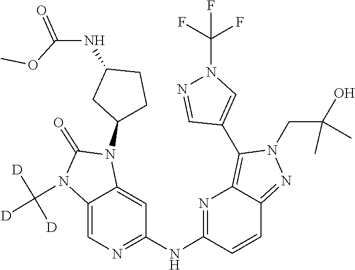 [SMILES: [2H]C([2H])([2H])n1c(=O)n([C@@H]2CC[C@@H](NC(=O)OC)C2)c2cc(Nc3ccc4nn(CC(C)(C)O)c(-c5cnn(C(F)(F)F)c5)c4n3)ncc21]

### Step 1. 1-(5-Chloro-3-(1-(trifluoromethyl)-1H-pyrazol-4-yl)-2H-pyrazolo[4,3-b]pyridin-2-yl)-2-methylpropan-2-ol

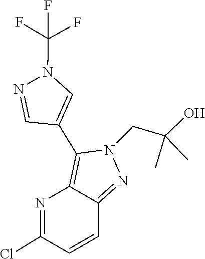 [SMILES: CC(C)(O)Cn1nc2ccc(Cl)nc2c1-c1cnn(C(F)(F)F)c1]

This compound was prepared according to the procedures described in Example 118, using the second peak isolated in Step 1. LCMS calculated for C₁₄H₁₄ClF₃N₅O (M+H)⁺: m/z=360.1; found: 360.1.

### Step 2. Methyl ((1R,3R)-3-(6-((2-(2-hydroxy-2-methylpropyl)-3-(1-(trifluoromethyl)-1H-pyrazol-4-yl)-2H-pyrazolo[4,3-b]pyridin-5-yl)amino)-3-(methyl-d₃)-2-oxo-2,3-dihydro-1H-imidazo[4,5-c]pyridin-1-yl)cyclopentyl)carbamate

This compound was prepared according to the procedures described in Example 91, with methyl ((1R,3R)-3-(6-amino-3-(methyl-d₃)-2-oxo-2,3-dihydro-1H-imidazo[4,5-c]pyridin-1-yl)cyclopentyl)carbamate replacing N-((1s,4s)-4-(6-amino-3-(methyl-d₃)-2-oxo-2,3-dihydro-1H-imidazo[4,5-c]pyridin-1-yl)-1-methylcyclohexyl)cyclopropanecarboxamide and 1-(5-chloro-3-(1-(trifluoromethyl)-1H-pyrazol-4-yl)-2H-pyrazolo[4,3-b]pyridin-2-yl)-2-methylpropan-2-ol replacing 5-chloro-2-methyl-3-(1-(trifluoromethyl)-1H-pyrazol-4-yl)-2H-pyrazolo[4,3-b]pyridine in Step 6. LCMS calculated for C₂₈H₂₉D₃F₃N₁₀O₄ (M+H)⁺: m/z=632.3; found: 632.3.

### Example 123. N-((1S,3R)-3-(6-((2-(2-Hydroxy-2-methylpropyl)-3-(1-(trifluoromethyl)-1H-pyrazol-4-yl)-2H-pyrazolo[4,3-b]pyridin-5-yl)amino)-3-(methyl-d₃)-2-oxo-2,3-dihydro-1H-imidazo[4,5-c]pyridin-1-yl)-1-methylcyclopentyl)cyclopropanecarboxamide

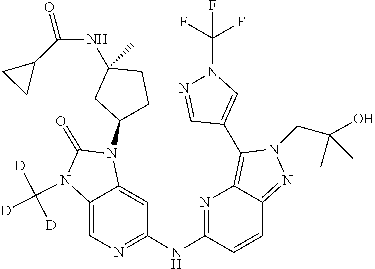 [SMILES: [2H]C([2H])([2H])n1c(=O)n([C@@H]2CC[C@](C)(NC(=O)C3CC3)C2)c2cc(Nc3ccc4nn(CC(C)(C)O)c(-c5cnn(C(F)(F)F)c5)c4n3)ncc21]

This compound was prepared according to the procedures described in Example 122, with N-((1S,3R)-3-(6-amino-3-(methyl-d₃)-2-oxo-2,3-dihydro-1H-imidazo[4,5-c]pyridin-1-yl)-1-methylcyclopentyl)cyclopropanecarboxamide replacing methyl ((1R,3R)-3-(6-amino-3-(methyl-d₃)-2-oxo-2,3-dihydro-1H-imidazo[4,5-c]pyridin-1-yl)cyclopentyl)carbamate in Step 3. LCMS calculated for C₃₁H₃₃D₃F₃N₁₀O₃ (M+H)⁺: m/z=656.3; found: 656.3

### Example 124. Methyl ((3R)-3-(6-((1-(2-hydroxy-2-methylpropyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)cyclopentyl-1-d)carbamate

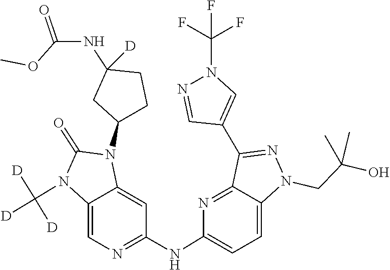 [SMILES: [2H]C1(NC(=O)OC)CC[C@@H](n2c(=O)n(C([2H])([2H])[2H])c3cnc(Nc4ccc5c(n4)c(-c4cnn(C(F)(F)F)c4)nn5CC(C)(C)O)cc32)C1]

This compound was prepared according to the procedures described in Example 118, with methyl ((3R)-3-(6-chloro-3-(methyl-d₃)-2-oxo-2,3-dihydro-1H-imidazo[4,5-c]pyridin-1-yl)cyclopentyl-1-d)carbamate replacing N-((1s,4s)-4-(6-chloro-3-(methyl-d₃)-2-oxo-2,3-dihydro-1H-imidazo[4,5-c]pyridin-1-yl)-1-methylcyclohexyl)cyclopropanecarboxamide in Step 3. The product was purified by prep-LCMS as the first peak of two diastereoisomers (XBridge C18 column, eluting with a gradient of acetonitrile/water containing 0.1% TFA, at flow rate of 60 mL/min). Retention time=9.7 min. LCMS calculated for C₂₈H₂₈D₄F₃N₁₀O₄ (M+H)⁺: m/z=633.3; found: 633.3.

### Example 125. Methyl ((3R)-3-(6-((1-(2-hydroxy-2-methylpropyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)cyclopentyl-1-d)carbamate

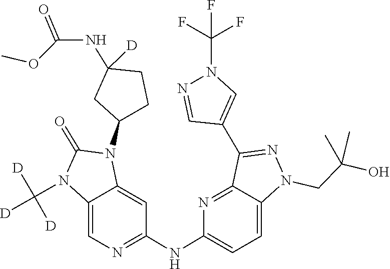 [SMILES: [2H]C1(NC(=O)OC)CC[C@@H](n2c(=O)n(C([2H])([2H])[2H])c3cnc(Nc4ccc5c(n4)c(-c4cnn(C(F)(F)F)c4)nn5CC(C)(C)O)cc32)C1]

This compound was prepared according to the procedures described in Example 118, with methyl ((3R)-3-(6-chloro-3-(methyl-d₃)-2-oxo-2,3-dihydro-1H-imidazo[4,5-c]pyridin-1-yl)cyclopentyl-1-d)carbamate replacing N-((1s,4s)-4-(6-chloro-3-(methyl-d₃)-2-oxo-2,3-dihydro-1H-imidazo[4,5-c]pyridin-1-yl)-1-methylcyclohexyl)cyclopropanecarboxamide in Step 3. The product was purified by prep-LCMS as the second peak of two diastereoisomers (XBridge C18 column, eluting with a gradient of acetonitrile/water containing 0.1% TFA, at flow rate of 60 mL/min). Retention time=10.5 min. LCMS calculated for C₂₈H₂₈D₄F₃N₁₀O₄ (M+H)⁺: m/z=633.3; found: 633.3.

### Example 126. N-((1s,4s)-4-(6-((1-((1r,4r)-4-Hydroxycyclohexyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)-1-methylcyclohexyl)cyclopropanecarboxamide

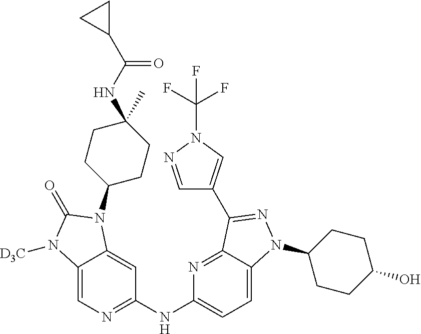 [SMILES: [2H]C([2H])([2H])n1c(=O)n([C@H]2CC[C@@](C)(NC(=O)C3CC3)CC2)c2cc(Nc3ccc4c(n3)c(-c3cnn(C(F)(F)F)c3)nn4[C@H]3CC[C@H](O)CC3)ncc21]

### Step 1. (1r,4r)-4-(5-Chloro-3-(1-(trifluoromethyl)-1H-pyrazol-4-yl)-1H-pyrazolo[4,3-b]pyridin-1-yl)cyclohexan-1-ol

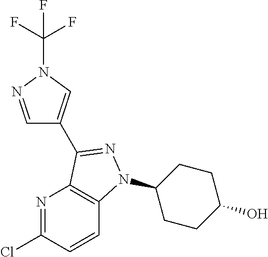 [SMILES: O[C@H]1CC[C@H](n2nc(-c3cnn(C(F)(F)F)c3)c3nc(Cl)ccc32)CC1]

To a vial containing 5-chloro-3-(1-(trifluoromethyl)-1H-pyrazol-4-yl)-1H-pyrazolo[4,3-b]pyridine (60 mg, 0.21 mmol) and (1s,4s)-4-((tert-butyldimethylsilyl)oxy)cyclohexyl methanesulfonate (193 mg, 0.63 mmol) in DMF (2.0 mL) was added potassium carbonate (115 mg, 0.83 mmol). The reaction was stirred at 100° C. for 6 h. The mixture was cooled down to r.t., the solids were filtered off, and the solvent was evaporated in vacuo. The crude material was treated with THF (2 mL) and 2N HCl (1 mL) and stirred at r.t. for 2 h. The mixture was diluted with EtOAc. The organic layer was washed with sat. NaHCO₃ solution, dried over sodium sulfate, filtered, and the solvents were evaporated in vacuo. The crude material was purified by Biotage Isolera to give the product. LCMS calculated for C₁₆H₁₆ClF₃N₅O (M+H)⁺: m/z=386.1; found 386.1.

### Step 2. N-((1s. 4s)-4-(6-((1-((1r. 4r)-4-Hydroxycyclohexyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)-1-methylcyclohexyl)cyclopropanecarboxamide

This compound was prepared according to the procedures described in Example 91, with (1r,4r)-4-(5-chloro-3-(1-(trifluoromethyl)-1H-pyrazol-4-yl)-1H-pyrazolo[4,3-b]pyridin-1-yl)cyclohexan-1-ol replacing 5-chloro-2-methyl-3-(1-(trifluoromethyl)-1H-pyrazol-4-yl)-2H-pyrazolo[4,3-b]pyridine in Step 6. LCMS calculated for C₃₄H₃₇D₃F₃N₁₀O₃ (M+H)⁺: m/z=696.3; found: 696.3.

### Example 127. N-((1s,4s)-1-Methyl-4-(3-(methyl-d₃)-6-((2-methyl-7-(morpholine-4-carbonyl)-3-(1-(trifluoromethyl)-1H-pyrazol-4-yl)-2H-pyrazolo[4,3-b]pyridin-5-yl)amino)-2-oxo-2,3-dihydro-1H-imidazo[4,5-c]pyridin-1-yl)cyclohexyl)cyclopropanecarboxamide

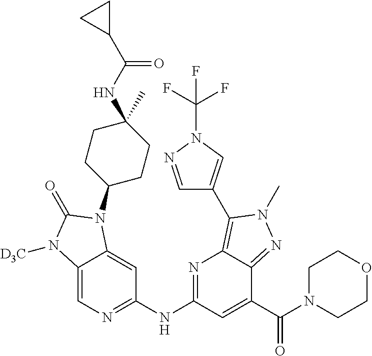 [SMILES: [2H]C([2H])([2H])n1c(=O)n([C@H]2CC[C@@](C)(NC(=O)C3CC3)CC2)c2cc(Nc3cc(C(=O)N4CCOCC4)c4nn(C)c(-c5cnn(C(F)(F)F)c5)c4n3)ncc21]

### Step 1. 5-((1-((1s,4s)-4-(Cyclopropanecarboxamido)-4-methylcyclohexyl)-3-(methyl-d₃)-2-oxo-2,3-dihydro-1H-imidazo[4,5-c]pyridin-6-yl)amino)-2-methyl-3-(1-(trifluoromethyl)-1H-pyrazol-4-yl)-2H-pyrazolo[4,3-b]pyridine-7-carboxylic acid

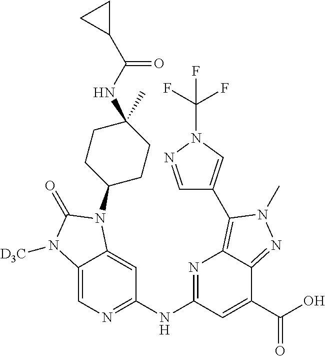 [SMILES: [2H]C([2H])([2H])n1c(=O)n([C@H]2CC[C@@](C)(NC(=O)C3CC3)CC2)c2cc(Nc3cc(C(=O)O)c4nn(C)c(-c5cnn(C(F)(F)F)c5)c4n3)ncc21]

This compound was prepared according to the procedures described in Example 91, with methyl 5-chloro-2-methyl-3-(1-(trifluoromethyl)-1H-pyrazol-4-yl)-2H-pyrazolo[4,3-b]pyridine-7-carboxylate replacing 5-chloro-2-methyl-3-(1-(trifluoromethyl)-1H-pyrazol-4-yl)-2H-pyrazolo[4,3-b]pyridine in Step 6. LCMS calculated for C₃₀H₂₉D₃F₃N₁₀O₄ (M+H)⁺: m/z=656.3; found: 656.3.

### Step 2. N-((1s,4s)-1-Methyl-4-(3-(methyl-d₃)-6-((2-methyl-7-(morpholine-4-carbonyl)-3-(1-(trifluoromethyl)-1H-pyrazol-4-yl)-2H-pyrazolo[4,3-b]pyridin-5-yl)amino)-2-oxo-2,3-dihydro-1H-imidazo[4,5-c]pyridin-1-yl)cyclohexyl)cyclopropanecarboxamide

To a vial containing 5-((1-((1s,4s)-4-(cyclopropanecarboxamido)-4-methylcyclohexyl)-3-(methyl-d₃)-2-oxo-2,3-dihydro-1H-imidazo[4,5-c]pyridin-6-yl)amino)-2-methyl-3-(1-(trifluoromethyl)-1H-pyrazol-4-yl)-2H-pyrazolo[4,3-b]pyridine-7-carboxylic acid (3 mg, 5 μmol), morpholine (2 mg, 0.02 mmol) and triethylamine (3 μl, 0.02 mmol) in acetonitrile (1.0 mL) at 0° C. was added propane phosphonic acid anhydride (T3P 50% in EtOAc, 3 mg, 9 μmol). The reaction was stirred at rt for 2 h and the product was purified by prep-LCMS (XBridge C18 column, eluting with a gradient of acetonitrile/water containing 0.1% TFA, at flow rate of 60 mL/min). LCMS calculated for C₃₄H₃₆D₃F₃N₁₁O₄ (M+H)⁺: m/z=725.3; found: 725.4.

### Example 128. Methyl ((1S,3R)-3-(6-((1-(2-hydroxy-2-methylpropyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)-1-methylcyclopentyl)carbamate

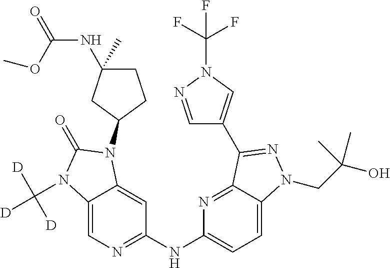 [SMILES: [2H]C([2H])([2H])n1c(=O)n([C@@H]2CC[C@](C)(NC(=O)OC)C2)c2cc(Nc3ccc4c(n3)c(-c3cnn(C(F)(F)F)c3)nn4CC(C)(C)O)ncc21]

### Step 1. Methyl ((1S,3R)-3-(6-chloro-3-(methyl-d₃)-2-oxo-2,3-dihydro-1H-imidazo[4,5-c]pyridin-1-yl)-1-methylcyclopentyl)carbamate

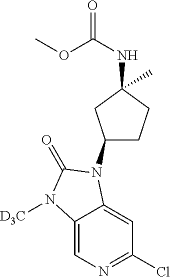 [SMILES: [2H]C([2H])([2H])n1c(=O)n([C@@H]2CC[C@](C)(NC(=O)OC)C2)c2cc(Cl)ncc21]

This compound was prepared according to the procedures described in Intermediate A, with tert-butyl ((1S,3R)-3-amino-1-methylcyclopentyl)carbamate replacing tert-butyl ((1R,3R)-3-aminocyclopentyl)carbamate in Step 1. LCMS calculated for C₁₅H₁₇D₃ClN₄O₃ (M+H)⁺: m/z=342.1; found: 342.1.

### Step 2. Methyl ((1S,3R)-3-(6-((1-(2-hydroxy-2-methylpropyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)-1-methylcyclopentyl) carbamate

This compound was prepared according to the procedures described in Example 118, with tert-butyl ((1S,3R)-3-(6-chloro-3-(methyl-d₃)-2-oxo-2,3-dihydro-1H-imidazo[4,5-c]pyridin-1-yl)-1-methylcyclopentyl)carbamate replacing N-((1s,4s)-4-(6-chloro-3-(methyl-d₃)-2-oxo-2,3-dihydro-1H-imidazo[4,5-c]pyridin-1-yl)-1-methylcyclohexyl)cyclopropanecarboxamide in Step 3. LCMS calculated for C₂₉H₃₁D₃F₃N₁₀O₄ (M+H)⁺: m/z=646.3; found: 646.4.

### Example 129. N-((1S,3R)-3-(6-((1-(2-Hydroxy-2-methylpropyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)-1-methylcyclopentyl)acetamide

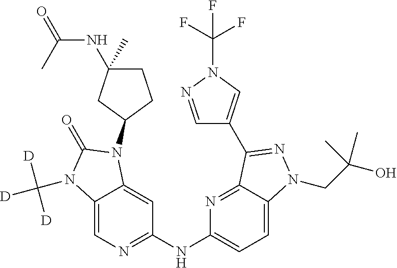 [SMILES: [2H]C([2H])([2H])n1c(=O)n([C@@H]2CC[C@](C)(NC(C)=O)C2)c2cc(Nc3ccc4c(n3)c(-c3cnn(C(F)(F)F)c3)nn4CC(C)(C)O)ncc21]

This compound was prepared according to the procedures described in Example 118, with N-((1S,3R)-3-(6-chloro-3-(methyl-d₃)-2-oxo-2,3-dihydro-1H-imidazo[4,5-c]pyridin-1-yl)-1-methylcyclopentyl)acetamide (Example 136), replacing N-((1s,4s)-4-(6-chloro-3-(methyl-d₃)-2-oxo-2,3-dihydro-1H-imidazo[4,5-c]pyridin-1-yl)-1-methylcyclohexyl)cyclopropanecarboxamide in Step 3. LCMS calculated for C₂₉H₃₁D₃F₃N₁₀O₃ (M+H)⁺: m/z=630.3; found: 630.3.

### Example 130. Methyl ((3R)-3-(6-((7-(2-hydroxypropan-2-yl)-3-(1-(trifluoromethyl)-1H-pyrazol-4-yl)-1H-pyrrolo[3,2-b]pyridin-5-yl)amino)-3-(methyl-d₃)-2-oxo-2,3-dihydro-1H-imidazo[4,5-c]pyridin-1-yl)cyclopentyl-1-d)carbamate

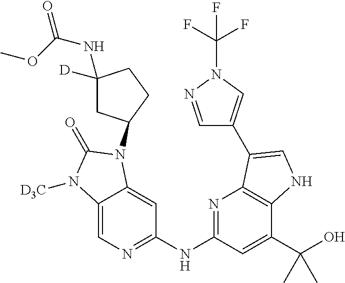 [SMILES: [2H]C1(NC(=O)OC)CC[C@@H](n2c(=O)n(C([2H])([2H])[2H])c3cnc(Nc4cc(C(C)(C)O)c5[nH]cc(-c6cnn(C(F)(F)F)c6)c5n4)cc32)C1]

### Step 1. 7-Bromo-5-chloro-1-((2-(trimethylsilyl)ethoxy)methyl)-1H-pyrrolo[3,2-b]pyridine

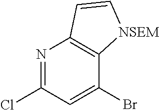 [SMILES: C[Si](C)(C)CCOCn1ccc2nc(Cl)cc(Br)c21]

To a vial containing 7-bromo-5-chloro-1H-pyrrolo[3,2-b]pyridine (1.0 g, 4.3 mmol) in THF (29 mL) at 0° C. was added sodium hydride (260 mg, 6.5 mmol, 60% dispersion in mineral oil) and left to stir. After 10 min, 2-(trimethylsilyl)ethoxymethyl chloride (840 μL, 4.7 mmol) was added and left to stir at 0° C. After 1 h, the reaction was quenched with sat. aq. NH₄Cl solution (50 mL) and extracted into CH₂Cl₂ (100 mL). The organic phase was washed with brine, dried over MgSO₄ and concentrated in vacuo. The obtained crude product was used directly in the next step without further purification. LCMS calculated for C₁₃H₁₉BrClN₂OSi (M+H)⁺: m/z=361.0/363.0; found 361.1/363.0.

### Step 2. 7-Bromo-5-chloro-3-iodo-1-((2-(trimethylsilyl)ethoxy)methyl)-1H-pyrrolo[3,2-b]pyridine

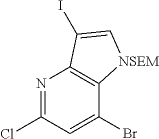 [SMILES: C[Si](C)(C)CCOCn1cc(I)c2nc(Cl)cc(Br)c21]

To a vial containing 7-bromo-5-chloro-1-((2-(trimethylsilyl)ethoxy)methyl)-1H-pyrrolo[3,2-b]pyridine (1.5 g, 4.2 mmol) in DMF (17 mL) was added N-iodosuccinimide (1.4 g. 6.3 mmol) and left to stir at rt. After 16 h, the reaction mixture was poured into a 0° C. sat. aq. sodium thiosulfate solution (50 mL) and the resulting precipitate was filtered, washed with water, and dried to provide a white solid. The crude residue was purified by Teledyne ISCO CombiFlash to give the desired product. LCMS calculated for C₁₃H₁₈BrClIN₂OSi (M+H)⁺: m/z=486.9/488.9; found 486.9/488.8.

### Step 3. 7-Bromo-5-chloro-3-(1-(trifluoromethyl)-1H-pyrazol-4-yl)-1-((2-(trimethylsilyl)ethoxy)methyl)-1H-pyrrolo[3,2-b]pyridine

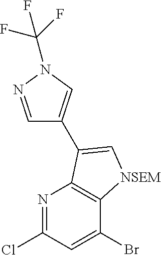 [SMILES: C[Si](C)(C)CCOCn1cc(-c2cnn(C(F)(F)F)c2)c2nc(Cl)cc(Br)c21]

A vial containing 7-bromo-5-chloro-3-iodo-1-((2-(trimethylsilyl)ethoxy)methyl)-1H-pyrrolo[3,2-b]pyridine (860 mg, 1.7 mmol), 4-(4,4,5,5-tetramethyl-1,3,2-dioxaborolan-2-yl)-1-(trifluoromethyl)-1H-pyrazole (570 mg, 2.2 mmol), tetrakis(triphenylphosphine)palladium(0) (200 mg, 0.18 mmol) and K₂CO₃ (730 mg, 5.3 mmol) was evacuated and backfilled with nitrogen three times, followed by the addition of 1,4-dioxane (7 mL) and water (1.5 mL). The reaction was sealed and heated to 65° C. for 16 h. After cooling to room temperature, the mixture was filtered through Celite and washed with CH₂Cl₂, followed by concentration of the filtrate in vacuo. The resulting residue was purified by Teledyne ISCO CombiFlash to give the desired product (865 mg, 99% yield). LCMS calculated for C₁₇H₂₀BrClF₃NOSi (M+H)⁺: m/z=495.0/497.0; found: 495.1/497.0.

### Step 4. 5-Chloro-7-(prop-1-en-2-yl)-3-(1-(trifluoromethyl)-1H-pyrazol-4-yl)-1-((2-(trimethylsilyl)ethoxy)methyl)-1H-pyrrolo[3,2-b]pyridine

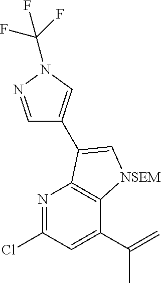 [SMILES: C=C(C)c1cc(Cl)nc2c(-c3cnn(C(F)(F)F)c3)cn(COCC[Si](C)(C)C)c12]

A vial containing 7-bromo-5-chloro-3-(1-(trifluoromethyl)-1H-pyrazol-4-yl)-1-((2-(trimethylsilyl)ethoxy)methyl)-1H-pyrrolo[3,2-b]pyridine (50 mg, 0.1 mmol), 4,4,5,5-tetramethyl-2-(prop-1-en-2-yl)-1,3,2-dioxaborolane (28 μL, 0.15 mmol), tetrakis(triphenylphosphine)palladium(0) (12 mg, 10 μmol) and Cs₂CO₃ (99 mg, 0.3 mmol) was evacuated and backfilled with nitrogen three times, followed by the addition of 1,4-dioxane (1 mL) and water (200 μL). The reaction was sealed and heated to 80° C. for 1 h. After cooling to room temperature, the mixture was filtered through Celite and washed with CH₂C₁₂, followed by concentration of the filtrate in vacuo. The resulting residue was purified by Teledyne ISCO CombiFlash to give the desired product. LCMS calculated for C₂₀H₂₅ClF₃N₄OSi (M+H)⁺: m/z=457.1/459.1; found: 457.2/459.2.

### Step 5. Methyl ((3R)-3-(6-amino-3-(methyl-d₃)-2-oxo-2,3-dihydro-1H-imidazo[4,5-c]pyridin-1-yl)cyclopentyl-1-d)carbamate

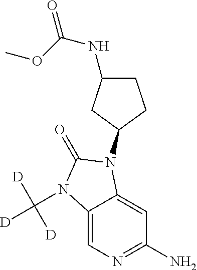 [SMILES: [2H]C([2H])([2H])n1c(=O)n([C@@H]2CCC(NC(=O)OC)C2)c2cc(N)ncc21]

A reaction vial containing methyl ((3R)-3-(6-chloro-3-(methyl-d₃)-2-oxo-2,3-dihydro-1H-imidazo[4,5-c]pyridin-1-yl)cyclopentyl-1-d)carbamate (1.1 g, 3.3 mmol), diphenylmethanimine (1.7 mL, 10 mmol), tris(dibenzylideneacetone)dipalladium(0) (460 mg, 0.5 mmol), 9,9-dimethyl-4,5-bis(diphenylphosphino)xanthene (580 mg, 1.0 mmol) and Cs₂CO₃ (3.3 g, 10 mmol) was evacuated and backfilled with nitrogen three times, followed by the addition of 1,4-dioxane (10 mL). The mixture was heated to 90° C. overnight. After cooling to room temperature, the mixture was filtered through Celite and washed with CH₂Cl₂, followed by concentration of the filtrate in vacuo. The crude residue was dissolved in MeOH (15 mL) and then hydroxylamine hydrochloride (1.2 g, 17 mmol) and potassium acetate (985 mg, 10 mmol) were added, and the reaction was left to stir at rt. After 2 h, the reaction mixture was concentrated in vacuo and the product was dissolved in CH₂Cl₂, filtered, and the filtrate was concentrated in vacuo. The resulting residue was purified by Teledyne ISCO CombiFlash to give the desired product. LCMS calculated for C₁₄H₁₆D₄N₅O₃ (M+H)⁺: m/z=310.2; found: 310.1

### Step 6. Methyl ((3R)-3-(3-(methyl-d₃)-2-oxo-6-((7-(prop-1-en-2-yl)-3-(1-(trifluoromethyl)-1H-pyrazol-4-yl)-1H-pyrrolo[3,2-b]pyridin-5-yl)amino)-2,3-dihydro-1H-imidazo[4,5-c]pyridin-1-yl)cyclopentyl-1-d)carbamate

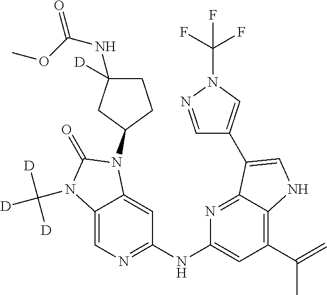 [SMILES: [2H]C1(NC(=O)OC)CC[C@@H](n2c(=O)n(C([2H])([2H])[2H])c3cnc(Nc4cc(C(=C)C)c5[nH]cc(-c6cnn(C(F)(F)F)c6)c5n4)cc32)C1]

A vial containing methyl ((3R)-3-(6-amino-3-(methyl-d₃)-2-oxo-2,3-dihydro-1H-imidazo[4,5-c]pyridin-1-yl)cyclopentyl-1-d)carbamate (45 mg, 0.14 mmol), 5-chloro-7-(prop-1-en-2-yl)-3-(1-(trifluoromethyl)-1H-pyrazol-4-yl)-1-((2-(trimethylsilyl)ethoxy)methyl)-1H-pyrrolo[3,2-b]pyridine (66 mg. 0.14 mmol), tris(dibenzylideneacetone)dipalladium(0) (13 mg, 15 μmol), 4,5-bis(diphenylphosphino)-9,9-dimethylxanthene (17 mg. 30 μmol) and Cs₂CO₃ (142 mg. 0.44 mmol) was evacuated and backfilled with nitrogen three times. Added 1,4-dioxane (1 mL) and the reaction mixture was heated to 100° C. for 16 h. After cooling to room temperature, the mixture was filtered through Celite and washed with CH₂Cl₂, followed by concentration of the filtrate in vacuo. The resulting residue was purified by Teledyne ISCO CombiFlash to give the desired intermediate. The material was re-dissolved in CH₂Cl₂ (1 mL) and TFA (1 mL) and left to stir at rt. After 2 h, the reaction mixture was concentrated in vacuo, and the crude residue was re-dissolved in MeOH (1 mL), THF (1 mL), and aq. NH₄OH (1 mL) and left to stir at rt. After 30 min, the reaction mixture was concentrated in vacuo, then diluted with sat. aq. NH₄Cl and extracted with 3:1 chloroforom/isopropyl alcohol to give the desired products as a mixture of diastereomers. LCMS calculated for C₂₈H₂₄D₄F₃N₉O₃ (M+H)⁺: m/z=600.3; found: 600.3.

### Step 7. Methyl ((3R)-3-(6-((7-(2-hydroxypropan-2-yl)-3-(1-(trifluoromethyl)-1H-pyrazol-4-yl)-1H-pyrrolo[3,2-b]pyridin-5-yl)amino)-3-(methyl-d₃)-2-oxo-2,3-dihydro-1H-imidazo[4,5-c]pyridin-1-yl)cyclopentyl-1-d)carbamate

To a vial containing methyl ((3R)-3-(3-(methyl-d₃)-2-oxo-6-((7-(prop-1-en-2-yl)-3-(1-(trifluoromethyl)-1H-pyrazol-4-yl)-1H-pyrrolo[3,2-b]pyridin-5-yl)amino)-2,3-dihydro-1H-imidazo[4,5-c]pyridin-1-yl)cyclopentyl-1-d)carbamate (34 mg, 0.06 mmol), iron(III) acetylacetonate (2 mg, 5.7 μmol), methyl 4-nitrobenzenesulfonate (15 mg, 0.07 mmol), and sodium bicarbonate (10 mg, 0.11 mmol) in MeOH (1 mL) at 0° C. was added phenylsilane (21 μL, 0.17 mmol). The reaction mixture was warmed to rt and left to stir. After 1 h, the reaction mixture was diluted with MeCN and purified by prep-LCMS (XBridge C18 column, eluting with a gradient of 22→40% acetonitrile in water containing 0.1% TFA, at flow rate of 60 mL/min over 12 min). The desired diastereomer was collected at a retention time of 10.6 min. LCMS calculated for C₂₈H₂₇D₄F₃N₉O₄ (M+H)⁺: m/z=618.3; found 618.3.

### Example 131. Methyl ((3R)-3-(6-((4-(2-hydroxypropan-2-yl)-1-(1-(trifluoromethyl)-1H-pyrazol-4-yl)-1H-pyrrolo[2,3-b]pyridin-6-yl)amino)-3-(methyl-d₃)-2-oxo-2,3-dihydro-1H-imidazo[4,5-c]pyridin-1-yl)cyclopentyl-1-d)carbamate

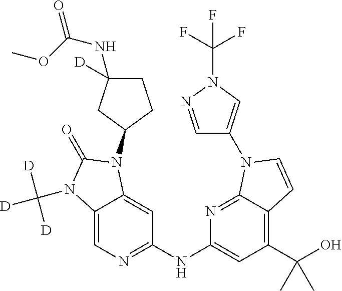 [SMILES: [2H]C1(NC(=O)OC)CC[C@@H](n2c(=O)n(C([2H])([2H])[2H])c3cnc(Nc4cc(C(C)(C)O)c5ccn(-c6cnn(C(F)(F)F)c6)c5n4)cc32)C1]

### Step 1. 4-Bromo-6-chloro-1-(1-(trifluoromethyl)-1H-pyrazol-4-yl)-1H-pyrrolo[2,3-b]pyridine

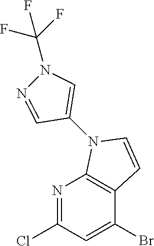 [SMILES: FC(F)(F)n1cc(-n2ccc3c(Br)cc(Cl)nc32)cn1]

A vial containing 4-bromo-6-chloro-1H-pyrrolo[2,3-b]pyridine (200 mg, 0.86 mmol), 4-(4,4,5,5-tetramethyl-1,3,2-dioxaborolan-2-yl)-1-(trifluoromethyl)-1H-pyrazole (270 mg, 1.0 mmol), and copper(II) acetate monohydrate (34 mg, 0.17 mmol) was evacuated and backfilled with oxygen three times, followed by the addition of CH₂Cl₂ (6 mL) and 1,8-diazabicyclo[5.4.0]undec-7-ene (130 μL, 0.86 mmol) and left to stir at rt. After 4 h, the reaction mixture was concentrated in vacuo, and the crude residue was purified by Teledyne ISCO CombiFlash to give the desired product (268 mg, 85% yield). LCMS calculated for C₁₁H₆BrClF₃N₄ (M+H)⁺: m/z=364.8/366.9; found 364.9/366.8.

### Step 2. 6-Chloro-4-(prop-1-en-2-yl)-1-(1-(trifluoromethyl)-1H-pyrazol-4-yl)-1H-pyrrolo[2,3-b]pyridine

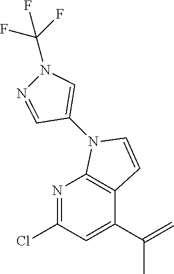 [SMILES: C=C(C)c1cc(Cl)nc2c1ccn2-c1cnn(C(F)(F)F)c1]

This compound was prepared according to the procedures described in Example 130, with 4-bromo-6-chloro-1-(1-(trifluoromethyl)-1H-pyrazol-4-yl)-1H-pyrrolo[2,3-b]pyridine replacing 7-bromo-5-chloro-3-(1-(trifluoromethyl)-1H-pyrazol-4-yl)-1-((2-(trimethylsilyl)ethoxy)methyl)-1H-pyrrolo[3,2-b]pyridine in Step 4. LCMS calculated for C₁₄H₁₁ClF₃N₄ (M+H)⁺: m/z=327.1/329.1; found: 327.0/329.0.

### Step 3. Methyl ((3R)-3-(3-(methyl-d₃)-2-oxo-6-((4-(prop-1-en-2-yl)-1-(1-(trifluoromethyl)-1H-pyrazol-4-yl)-1H-pyrrolo[2,3-b]pyridin-6-yl)amino)-2,3-dihydro-1H-imidazo[4,5-c]pyridin-1-yl)cyclopentyl-1-d)carbamate

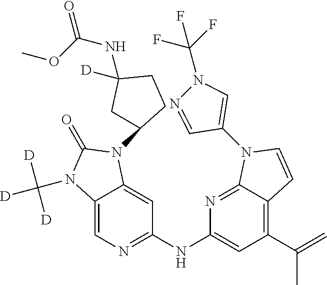 [SMILES: [2H]C1(NC(=O)OC)CC[C@@H](n2c(=O)n(C([2H])([2H])[2H])c3cnc(Nc4cc(C(=C)C)c5ccn(-c6cnn(C(F)(F)F)c6)c5n4)cc32)C1]

This compound was prepared according to the procedures described in Example 132, with 6-chloro-4-(prop-1-en-2-yl)-1-(1-(trifluoromethyl)-1H-pyrazol-4-yl)-1H-pyrrolo[2,3-b]pyridine replacing 5-chloro-1-(tetrahydro-2H-pyran-4-yl)-3-(1-(trifluoromethyl)-1H-pyrazol-4-yl)-1H-pyrazolo[4,3-b]pyridine in Step 3. LCMS calculated for C₂₈H₂₅D₄F₃N₉O₃ (M+H)⁺: m/z=600.3; found: 600.3.

### Step 4. Methyl ((3R)-3-(6-((4-(2-hydroxypropan-2-yl)-1-(1-(trifluoromethyl)-1H-pyrazol-4-yl)-1H-pyrrolo[2,3-b]pyridin-6-yl)amino)-3-(methyl-d₃)-2-oxo-2,3-dihydro-1H-imidazo[4,5-c]pyridin-1-yl)cyclopentyl-1-d)carbamate

This compound was prepared according to the procedures described in Example 130, with methyl ((3R)-3-(3-(methyl-d₃)-2-oxo-6-((4-(prop-1-en-2-yl)-1-(1-(trifluoromethyl)-1H-pyrazol-4-yl)-1H-pyrrolo[2,3-b]pyridin-6-yl)amino)-2,3-dihydro-1H-imidazo[4,5-c]pyridin-1-yl)cyclopentyl-1-d)carbamate replacing methyl ((3R)-3-(3-(methyl-d₃)-2-oxo-6-((7-(prop-1-en-2-yl)-3-(1-(trifluoromethyl)-1H-pyrazol-4-yl)-1H-pyrrolo[3,2-b]pyridin-5-yl)amino)-2,3-dihydro-1H-imidazo[4,5-c]pyridin-1-yl)cyclopentyl-1-d)carbamate in Step 7 and was purified by prep-LCMS (XBridge C18 column, eluting with a gradient of 22→40% acetonitrile in water containing 0.1% TFA, at flow rate of 60 mL/min over 12 min). The desired diastereomer was collected at a retention time of 9.8 min. LCMS calculated for C₂₈H₂₇D₄F₃N₉O₄ (M+H)⁺: m/z=618.3; found: 618.3. ¹H NMR (400 MHZ, DMSO-d₆) δ 9.04 (s, 1H), 8.73 (s, 1H), 8.09 (s, 1H), 7.73 (d, J=3.7 Hz, 1H), 7.51 (s, 1H), 7.35 (s, 1H), 7.21 (s, 1H), 6.91 (d, J=3.7 Hz, 1H), 4.80-4.68 (m, 1H), 3.52 (s, 3H), 2.21 (dd, J=12.5, 8.3 Hz, 1H), 2.17-2.08 (m, 1H), 2.07-1.96 (m, 2H), 1.96-1.86 (m, 1H), 1.81-1.70 (m, 1H), 1.62 (s, 6H).

### Example 132. Methyl ((3R)-3-(3-(methyl-d₃)-2-oxo-6-((1-(tetrahydro-2H-pyran-4-yl)-3-(1-(trifluoromethyl)-1H-pyrazol-4-yl)-1H-pyrazolo[4,3-b]pyridin-5-yl)amino)-2,3-dihydro-1H-imidazo[4,5-c]pyridin-1-yl)cyclopentyl-1-d)carbamate

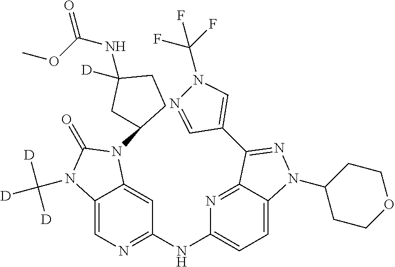 [SMILES: [2H]C1(NC(=O)OC)CC[C@@H](n2c(=O)n(C([2H])([2H])[2H])c3cnc(Nc4ccc5c(n4)c(-c4cnn(C(F)(F)F)c4)nn5C4CCOCC4)cc32)C1]

### Step 1. 5-Chloro-3-(1-(trifluoromethyl)-1H-pyrazol-4-yl)-1H-pyrazolo[4,3-b]pyridine

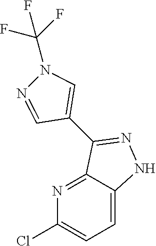 [SMILES: FC(F)(F)n1cc(-c2n[nH]c3ccc(Cl)nc23)cn1]

A vial containing 5-chloro-3-iodo-1H-pyrazolo[4,3-b]pyridine (250 mg, 0.9 mmol), 4-(4,4,5,5-tetramethyl-1,3,2-dioxaborolan-2-yl)-1-(trifluoromethyl)-1H-pyrazole (281 mg, 1.1 mmol), tetrakis(triphenylphosphine)palladium(0) (103 mg, 0.09 mmol) and K₂CO₃ (371 mg, 2.7 mmol) was evacuated and backfilled with nitrogen three times, followed by the addition of 1,4-dioxane (5 mL) and water (1 mL). The reaction was sealed and heated to 70° C. for 16 h. After cooling to rt, the mixture was filtered through Celite and washed with CH₂Cl₂, followed by concentration of the filtrate in vacuo. The resulting residue was purified by Teledyne ISCO CombiFlash to give the desired product. LCMS calculated for C₁₀H₆ClF₃N₅ (M+H)⁺: m/z=288.0/290.0; found: 288.0/290.0.

### Step 2. 5-Chloro-1-(tetrahydro-2H-pyran-4-yl)-3-(1-(trifluoromethyl)-1H-pyrazol-4-yl)-1H-pyrazolo[4,3-b]pyridine

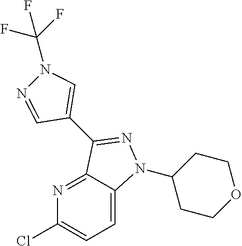 [SMILES: FC(F)(F)n1cc(-c2nn(C3CCOCC3)c3ccc(Cl)nc23)cn1]

To a vial containing 5-chloro-3-(1-(trifluoromethyl)-1H-pyrazol-4-yl)-1H-pyrazolo[4,3-b]pyridine (50 mg, 0.2 mmol) and Cs₂CO₃ (227 mg, 0.7 mmol) as a suspension in DMF (500 μL) was added 4-bromotetrahydro-2H-pyran (86 mg, 0.52 mmol), which was then sealed and heated to 100° C. for 16 h. After cooling to rt, the mixture was diluted with EtOAc and water and extracted three times with EtOAc. The combined organics were back-extracted three times with water, dried over MgSO₄, filtered and concentrated in vacuo. The crude residue was purified by Teledyne ISCO CombiFlash to give the desired product as the major regioisomer. LCMS calculated for C₁₅H₁₄ClF₃N₅O (M+H)⁺: m/z=372.1/374.1; found: 372.1/374.1.

### Step 3. Methyl ((3R)-3-(3-(methyl-d₃)-2-oxo-6-((1-(tetrahydro-2H-pyran-4-yl)-3-(1-(trifluoromethyl)-1H-pyrazol-4-yl)-1H-pyrazolo[4,3-b]pyridin-5-yl)amino)-2,3-dihydro-1H-imidazo[4,5-c]pyridin-1-yl)cyclopentyl-1-d)carbamate

A vial containing methyl ((3R)-3-(6-amino-3-(methyl-d₃)-2-oxo-2,3-dihydro-1H-imidazo[4,5-c]pyridin-1-yl)cyclopentyl-1-d)carbamate (20 mg, 65 μmol), 5-chloro-1-(tetrahydro-2H-pyran-4-yl)-3-(1-(trifluoromethyl)-1H-pyrazol-4-yl)-1H-pyrazolo[4,3-b]pyridine (23 mg, 62 μmol), tris(dibenzylideneacetone)dipalladium(0) (6 mg, 6 μmol), 4,5-bis(diphenylphosphino)-9,9-dimethylxanthene (7 mg, 12 μmol) and Cs₂CO₃ (60 mg, 0.2 mmol) was evacuated and backfilled with nitrogen three times. Added 1,4-dioxane (1 mL) and the reaction mixture was heated to 100° C. for 16 h. After cooling to rt, the mixture was filtered through Celite and washed with CH₂Cl₂, followed by concentration of the filtrate in vacuo. The crude residue was diluted with MeCN and purified by prep-LCMS (XBridge C18 column, eluting with a gradient of 22→40% acetonitrile in water containing 0.1% TFA, at flow rate of 60 mL/min over 12 min). The desired diastereomer was collected at a retention time of 9.6 min. LCMS calculated for C₂₉H₂₈D₄F₃N₁₀O₄ (M+H)⁺: m/z=645.3; found: 645.3.

### Example 133. Methyl ((3R)-3-(6-((1-((1-cyanocyclopropyl)methyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)cyclopentyl-1-d)carbamate

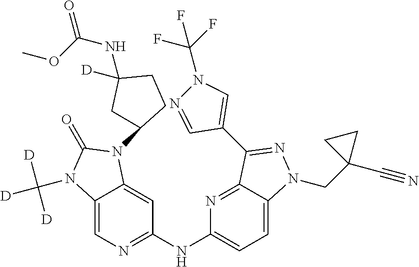 [SMILES: [2H]C1(NC(=O)OC)CC[C@@H](n2c(=O)n(C([2H])([2H])[2H])c3cnc(Nc4ccc5c(n4)c(-c4cnn(C(F)(F)F)c4)nn5CC4(C#N)CC4)cc32)C1]

This compound was prepared according to the procedures described in Example 132, with 1-(bromomethyl)cyclopropane-1-carbonitrile replacing 4-bromotetrahydro-2H-pyran in Step 2, isolating 1-((5-chloro-3-(1-(trifluoromethyl)-1H-pyrazol-4-yl)-1H-pyrazolo[4,3-b]pyridin-1-yl)methyl)cyclopropane-1-carbonitrile as the major regioisomer from alkylation. Purified by prep-LCMS (XBridge C18 column, eluting with a gradient of 22→40% acetonitrile in water containing 0.1% TFA, at flow rate of 60 mL/min over 12 min). The desired diastereomer was collected at a retention time of 9.3 min. LCMS calculated for C₂₉H₂₅D₄F₃N₁₁O₃ (M+H)⁺: m/z=640.3; found: 640.4.

### Example 134. Methyl ((3R)-3-(6-((2-((1-cyanocyclopropyl)methyl)-3-(1-(trifluoromethyl)-1H-pyrazol-4-yl)-2H-pyrazolo[4,3-b]pyridin-5-yl)amino)-3-(methyl-d₃)-2-oxo-2,3-dihydro-1H-imidazo[4,5-c]pyridin-1-yl)cyclopentyl-1-d)carbamate

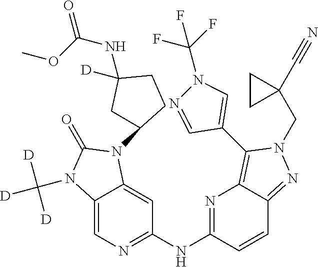 [SMILES: [2H]C1(NC(=O)OC)CC[C@@H](n2c(=O)n(C([2H])([2H])[2H])c3cnc(Nc4ccc5nn(CC6(C#N)CC6)c(-c6cnn(C(F)(F)F)c6)c5n4)cc32)C1]

This compound was prepared according to the procedures described in Example 132, with 1-(bromomethyl)cyclopropane-1-carbonitrile replacing 4-bromotetrahydro-2H-pyran in Step 2, isolating 1-((5-chloro-3-(1-(trifluoromethyl)-1H-pyrazol-4-yl)-2H-pyrazolo[4,3-b]pyridin-2-yl)methyl)cyclopropane-1-carbonitrile as the minor regioisomer from alkylation. Purified by prep-LCMS (XBridge C18 column, eluting with a gradient of 22→40% acetonitrile in water containing 0.1% TFA, at flow rate of 60 mL/min over 12 min). The desired diastereomer was collected at a retention time of 9.4 min. LCMS calculated for C₂₉H₂₅D₄F₃N₁₁O₃ (M+H)⁺: m/z=640.3; found: 640.3.

### Example 135. Methyl ((1R,3R)-3-(3-(methyl-d₃)-6-((6-methyl-4-(morpholine-4-carbonyl)-8-(trifluoromethyl)quinolin-2-yl)amino)-2-oxo-2,3-dihydro-1H-imidazo[4,5-c]pyridin-1-yl)cyclopentyl)carbamate

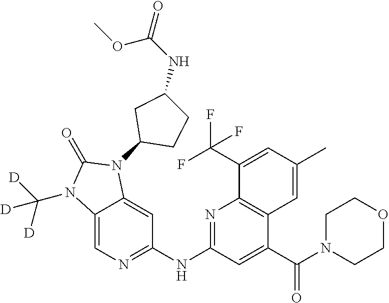 [SMILES: [2H]C([2H])([2H])n1c(=O)n([C@@H]2CC[C@@H](NC(=O)OC)C2)c2cc(Nc3cc(C(=O)N4CCOCC4)c4cc(C)cc(C(F)(F)F)c4n3)ncc21]

This compound was prepared according to the procedures described in Example 52, with 5-chloro-7-(trifluoromethyl)indoline-2,3-dione replacing 7-(trifluoromethyl)indoline-2,3-dione. LCMS calculated for C₃₀H₃₀D₃F₃N₇O₅ (M+H)⁺: m/z=631.3; found: 631.3. ¹H NMR (400 MHZ, DMSO-d₆) δ 11.13 (s, 1H), 8.33 (s, 1H), 8.03 (s, 1H), 7.96 (s, 1H), 7.75 (s, 1H), 7.34 (s, 1H), 4.89 (pent, J=8.4 Hz, 1H), 4.26 (pent, J=7.0 Hz, 1H), 3.88-3.62 (m, 4H), 3.55 (s, 3H), 3.49 (s, 2H), 3.23 (d, J=17.8 Hz, 2H), 2.55 (s, 3H), 2.43-2.31 (m, 1H), 2.29-2.04 (m, 3H), 2.00-1.88 (m, 1H), 1.76-1.55 (m, 1H).

### Example 136. N-((1S,3R)-3-(6-((6-(5-Chlorothiazol-2-yl)-4-(2-hydroxypropan-2-yl)pyridin-2-yl)amino)-3-(methyl-d₃)-2-oxo-2,3-dihydro-1H-imidazo[4,5-c]pyridin-1-yl)-1-methylcyclopentyl)acetamide

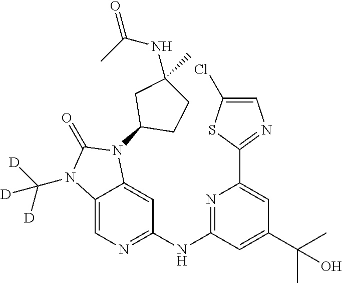 [SMILES: [2H]C([2H])([2H])n1c(=O)n([C@@H]2CC[C@](C)(NC(C)=O)C2)c2cc(Nc3cc(C(C)(C)O)cc(-c4ncc(Cl)s4)n3)ncc21]

This compound was prepared according to the procedures described in Example 92, with N-((1S,3R)-3-(6-chloro-3-(methyl-d₃)-2-oxo-2,3-dihydro-1H-imidazo[4,5-c]pyridin-1-yl)-1-methylcyclopentyl)acetamide replacing intermediate A in Step 6. LCMS calculated for C₂₋₆H₂₈D₃ClN₇O₃S (M+H)⁺: m/z=559.2; found 559.3.

### Example 137. 4-(6-((4-(2-Hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)-N-(methyl-d₃)bicyclo[2.2.2]octane-1-carboxamide

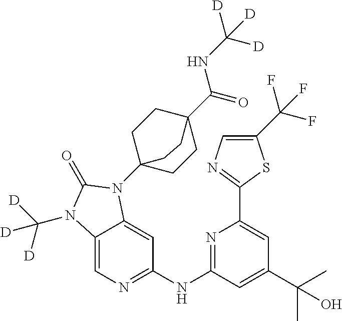 [SMILES: [2H]C([2H])([2H])NC(=O)C12CCC(n3c(=O)n(C([2H])([2H])[2H])c4cnc(Nc5cc(C(C)(C)O)cc(-c6ncc(C(F)(F)F)s6)n5)cc43)(CC1)CC2]

### Step 1. Methyl 2-bromo-6-((2,4-dimethoxybenzyl)amino)isonicotinate

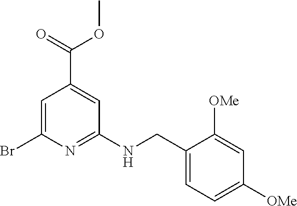 [SMILES: COC(=O)c1cc(Br)nc(NCc2ccc(OC)cc2OC)c1]

To a vial containing methyl 2,6-dibromoisonicotinate (2.95 g, 10 mmol) and (2,4-dimethoxyphenyl)methanamine (2.0 mL, 13 mmol) was added DMSO (10 mL), followed by DIPEA (2.7 mL, 15 mmol). The reaction was stirred at 100° C. for 2 h. The reaction mixture was cooled down and quenched by the addition of water and extracted with EtOAc. The organic layer was dried over Na₂SO₄, filtered, and the solvents were evaporated in vacuo. The crude material was purified by Biotage Isolera. LCMS calculated for C₁₆H₁₈BrN₂O₃ (M+H)⁺: m/z=381.0; found: 381.1.

### Step 2. Methyl 2-((2,4-dimethoxybenzyl)amino)-6-(5-(trifluoromethyl)thiazol-2-yl)isonicotinate

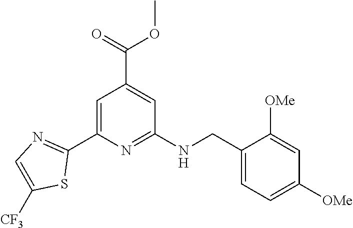 [SMILES: COC(=O)c1cc(NCc2ccc(OC)cc2OC)nc(-c2ncc(C(F)(F)F)s2)c1]

This compound was prepared according to the procedures described in Example 105, with methyl 2-bromo-6-((2,4-dimethoxybenzyl)amino)isonicotinate replacing 6-bromo-4-chloropyridin-2-amine in Step 2. LCMS calculated for C₂₀H₁₉F₃N₃O₄S (M+H)⁺: m/z=454.2; found: 454.2.

### Step 3. 2-(2-Amino-6-(5-(trifluoromethyl)thiazol-2-yl)pyridin-4-yl)propan-2-ol

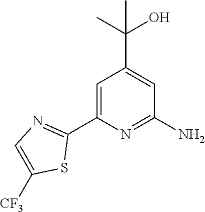 [SMILES: CC(C)(O)c1cc(N)nc(-c2ncc(C(F)(F)F)s2)c1]

To a flask containing methyl 2-((2,4-dimethoxybenzyl)amino)-6-(5-(trifluoromethyl)thiazol-2-yl)isonicotinate (2.0 g, 4.4 mmol) in THF (10 mL) was added dropwise methylmagnesium bromide (7.4 mL, 22.0 mmol, 3.0 M in Et₂O) at 0° C. The reaction was stirred at r.t for 15 min. The mixture was quenched with sat. NH₄Cl and extracted with EtOAc. The organic layers were combined, dried over Na₂SO₄, filtered, and the solvents were evaporated in vacuo. The crude material was then dissolved in TFA (5 mL) and stirred at 100° C. for 10 min. The reaction mixture was cooled to r.t, concentrated to remove volatiles, diluted with EtOAc and neutralized with sat. NaHCO₃. The organic layer was subsequently washed with water and brine, dried over Na₂SO₄, filtered, and the solvents were evaporated in vacuo. The crude material was purified by Biotage Isolera. LCMS calculated for C₁₂H₁₃F₃N₃OS (M+H)⁺: m/z=304.1; found 304.1.

### Step 4. Methyl 4-(6-chloro-3-(methyl-d₃)-2-oxo-2,3-dihydro-1H-imidazo[4,5-c]pyridin-1-yl)bicyclo[2.2.2]octane-1-carboxylate

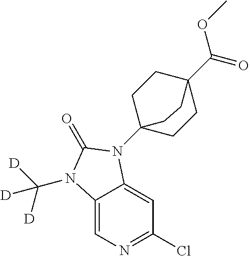 [SMILES: [2H]C([2H])([2H])n1c(=O)n(C23CCC(C(=O)OC)(CC2)CC3)c2cc(Cl)ncc21]

The title compound was prepared according to the procedures described in Intermediate A, Step 1-4 with methyl 4-aminobicyclo[2.2.2]octane-1-carboxylate replacing tert-butyl ((1R,3R)-3-aminocyclopentyl)carbamate. LCMS calculated for C₁₇H₁₈D₃ClN₃O₃ (M+H)⁺: m/z=353.1; found: 353.2.

### Step 5. Methyl 4-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)bicyclo[2.2.2]octane-1-carboxylate

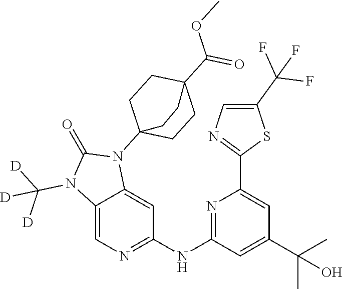 [SMILES: [2H]C([2H])([2H])n1c(=O)n(C23CCC(C(=O)OC)(CC2)CC3)c2cc(Nc3cc(C(C)(C)O)cc(-c4ncc(C(F)(F)F)s4)n3)ncc21]

A reaction vial containing methyl 4-(6-chloro-3-(methyl-d₃)-2-oxo-2,3-dihydro-1H-imidazo[4,5-c]pyridin-1-yl)bicyclo[2.2.2]octane-1-carboxylate (230 mg, 0.60 mmol), 2-(2-amino-6-(5-(trifluoromethyl)thiazol-2-yl)pyridin-4-yl)propan-2-ol (180 mg, 0.60 mmol), Pd₂(dba)₃ (109 mg, 0.12 mmol), xantphos (138 mg, 0.24 mmol) and cesium carbonate (580 mg, 1.8 mmol) was evacuated and backfilled with nitrogen. 1,4-Dioxane (7 mL) was added to the reaction mixture, which was then stirred at 110° C. for 3 h. The reaction mixture was cooled to r.t and diluted with EtOAc. The organic layer was subsequently washed with water and brine, dried over Na₂SO₄, filtered, and the solvents were evaporated in vacuo. The crude material was purified by Biotage Isolera. LCMS calculated for C₂₉H₂₉D₃F₃N₆O₄S (M+H)⁺: m/z=620.2; found 620.3.

### Step 6. 4-(6-((4-(2-Hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)bicyclo[2.2.2]octane-1-carboxylic acid

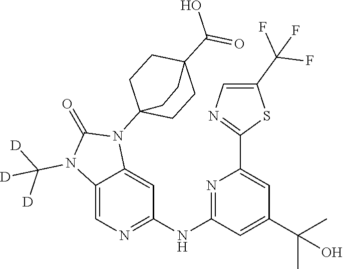 [SMILES: [2H]C([2H])([2H])n1c(=O)n(C23CCC(C(=O)O)(CC2)CC3)c2cc(Nc3cc(C(C)(C)O)cc(-c4ncc(C(F)(F)F)s4)n3)ncc21]

To a reaction vial containing methyl 4-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)bicyclo[2.2.2]octane-1-carboxylate (60 mg, 0.10 mmol) in MeOH (1 mL) and THF (1 mL) was added aqueous solution of LiOH (23 mg, 1.0 mmol) and stirred at r.t for 1 h. The mixture was concentrated in vacuo, then the solid was collected by filtration and washed with water. The crude material was used in the next step without further purification. LCMS calculated for C₂₈H₂₇D₃F₃N₆O₄S (M+H)⁺: m/z=606.2; found 606.2.

### Step 7. 4-(6-((4-(2-Hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)-N-(methyl-d₃)bicyclo[2.2.2]octane-1-carboxamide

To a vial containing 4-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)bicyclo[2.2.2]octane-1-carboxylic acid (10 mg, 0.017 mmol), HATU (9 mg, 0.025 mmol), DIPEA (9 μl, 0.05 mmol) and methan-d₃-amine (3 mg, 0.09 mmol) was added DMF (330 μl). The reaction was stirred at 60° C. for 20 min. The reaction mixture was cooled to r.t, diluted with MeCN and purified by prep-LCMS (XBridge C18 column, eluting with a gradient of acetonitrile/water containing 0.1% TFA, at flow rate of 60 mL/min. LCMS calculated for C₂₉H₂₇D₆F₃N₇O₃S (M+H)⁺: m/z=622.3; found: 622.3. ¹H NMR (400 MHZ, DMSO-d₆) 10.49 (s, 1H), 8.61 (s, 1H), 8.27 (s, 1H), 7.90 (s, 1H), 7.69 (s, 1H), 7.54 (s, 1H), 7.38 (s, 1H), 5.44 (s, 1H), 2.43-2.31 (m, 6H), 1.97-1.80 (m, 6H), 1.49 (s, 6H).

### Example 138. N-Cyclopropyl-4-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)bicyclo[2.2.2]octane-1-carboxamide

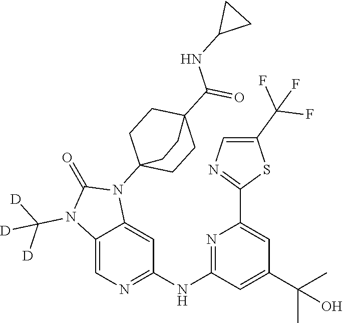 [SMILES: [2H]C([2H])([2H])n1c(=O)n(C23CCC(C(=O)NC4CC4)(CC2)CC3)c2cc(Nc3cc(C(C)(C)O)cc(-c4ncc(C(F)(F)F)s4)n3)ncc21]

This compound was prepared according to the procedures described in Example 137, with cyclopropanamine replacing methan-d₃-amine in Step 5. LCMS calculated for C₃₁H₃₂D₃F₃N₇O₃S (M+H)⁺: m/z=645.3; found 645.3. ¹H NMR (400 MHZ, DMSO-d₆) δ 10.17 (s, 1H), 8.60 (s, 1H), 8.19 (s, 1H), 7.86 (d, J=1.4 Hz, 1H), 7.75 (s, 1H), 7.61 (s, 1H), 7.38 (d, J=4.1 Hz, 1H), 5.44 (s, 1H), 2.65-2.54 (m, 1H), 2.43-2.28 (m, 6H), 2.00-1.75 (m, 6H), 1.48 (s, 6H), 0.73-0.47 (m, 2H), 0.45-0.34 (m, 2H).

### Example 139. 4-(6-((4-(2-Hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)bicyclo[2.2.2]octane-1-carboxamide

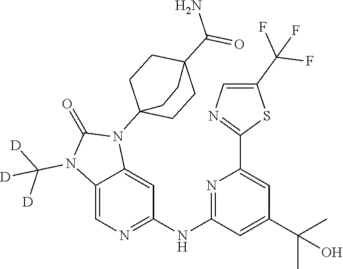 [SMILES: [2H]C([2H])([2H])n1c(=O)n(C23CCC(C(N)=O)(CC2)CC3)c2cc(Nc3cc(C(C)(C)O)cc(-c4ncc(C(F)(F)F)s4)n3)ncc21]

This compound was prepared according to the procedures described in Example 137, with ammonia (0.5 M in 1,4-dioxane) replacing methan-d₃-amine in Step 7. LCMS calculated for C₂₈H₂₈D₃F₃N₇O₃S (M+H)⁺: m/z=605.2; found 605.3

### Example 140. N-(4-(6-((4-(2-Hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)bicyclo[2.2.2]octan-1-yl)acetamide

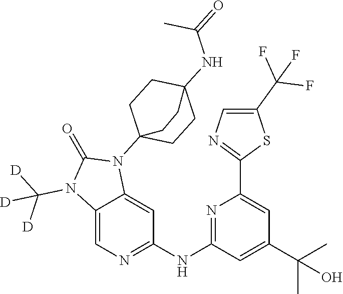 [SMILES: [2H]C([2H])([2H])n1c(=O)n(C23CCC(NC(C)=O)(CC2)CC3)c2cc(Nc3cc(C(C)(C)O)cc(-c4ncc(C(F)(F)F)s4)n3)ncc21]

This compound was prepared according to the procedures described in Example 137, with N-(4-(6-chloro-3-(methyl-d₃)-2-oxo-2,3-dihydro-1H-imidazo[4,5-c]pyridin-1-yl)bicyclo[2.2.2]octan-1-yl)acetamide replacing methyl 4-(6-chloro-3-(methyl-d₃)-2-oxo-2,3-dihydro-1H-imidazo[4,5-c]pyridin-1-yl)bicyclo[2.2.2]octane-1-carboxylate in Step 7. LCMS calculated for C₂₉H₃₀D₃F₃N₂O₃S (M+H)⁺: m/z=619.2; found 619.3.

### Example 141. 4-(6-((4-(2-Hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)bicyclo[2.2.1]heptane-1-carboxamide

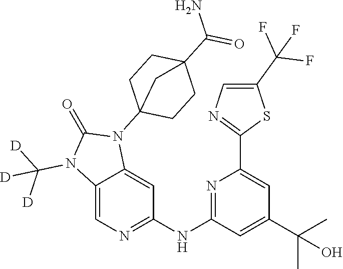 [SMILES: [2H]C([2H])([2H])n1c(=O)n(C23CCC(C(N)=O)(CC2)C3)c2cc(Nc3cc(C(C)(C)O)cc(-c4ncc(C(F)(F)F)s4)n3)ncc21]

This compound was prepared according to the procedures described in Example 137, with methyl 4-aminobicyclo[2.2.1]heptane-1-carboxylate replacing methyl 4-aminobicyclo[2.2.2]octane-1-carboxylate in Step 4. LCMS calculated for C₂₇H₂₆D₃F₃N₇O₃S (M+H)⁺: m/z=591.2; found 591.3. ¹H NMR (400 MHZ, DMSO-d₆) δ 10.34 (s, 1H), 8.60 (s, 1H), 8.23 (s, 1H), 7.87 (s, 1H), 7.63 (s, 1H), 7.60 (s, 1H), 7.17 (s, 1H), 6.92 (s, 1H), 5.43 (s, 1H), 2.50-2.34 (m, 4H), 2.08-1.88 (m, 4H), 1.80-1.62 (m, 2H), 1.49 (s, 6H).

### Example 142. N-(4-(6-((4-(2-Hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)bicyclo[2.2.1]heptan-1-yl)acetamide

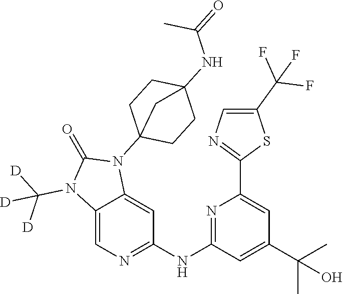 [SMILES: [2H]C([2H])([2H])n1c(=O)n(C23CCC(NC(C)=O)(CC2)C3)c2cc(Nc3cc(C(C)(C)O)cc(-c4ncc(C(F)(F)F)s4)n3)ncc21]

This compound was prepared according to the procedures described in Example 137, with tert-butyl (4-aminobicyclo[2.2.1]heptan-1-yl)carbamate replacing methyl 4-aminobicyclo[2.2.2]octane-1-carboxylate in Step 4. LCMS calculated for C₂₈H₂₈D₃F₃N₇O₃S (M+H)⁺: m/z=605.3; found 605.3. ¹H NMR (400 MHZ, DMSO-d₆) 10.51 (s, 1H), 8.61 (s, 1H), 8.26 (s, 1H), 8.07 (s, 1H), 7.90 (d, J=1.4 Hz, 1H), 7.59 (s, 1H), 7.53 (s, 1H), 5.34 (s, 1H), 2.64 (s, 2H), 2.49-2.41 (m, 2H), 2.12-1.88 (m, 4H), 1.80 (s, 3H), 1.78-1.64 (m, 2H), 1.49 (s, 6H).

### Example 143. N-((1S,3R)-3-(6-((4-(2-Hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)-1-methylcyclopentyl)propionamide

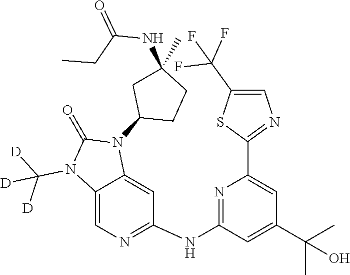 [SMILES: [2H]C([2H])([2H])n1c(=O)n([C@@H]2CC[C@](C)(NC(=O)CC)C2)c2cc(Nc3cc(C(C)(C)O)cc(-c4ncc(C(F)(F)F)s4)n3)ncc21]

This compound was prepared according to the procedures described in Example 137, with tert-butyl ((1S,3R)-3-amino-1-methylcyclopentyl)carbamate replacing methyl 4-aminobicyclo[2.2.2]octane-1-carboxylate in Step 4. LCMS calculated for C₂₉H₃₀D₃F₃N₇O₃S (M+H)⁺: m/z=607.2; found 607.3. ¹H NMR (400 MHZ, DMSO-d₆) δ 10.44 (s, 1H), 8.59 (s, 1H), 8.26 (s, 1H), 7.85 (s, 1H), 7.79 (s, 1H), 7.45 (s, 1H), 5.39 (s, 1H), 4.84 (p, J=8.8 Hz, 1H), 2.61-2.47 (m, 1H), 2.42-2.21 (m, 2H), 2.15-1.87 (m, 4H), 1.73-1.61 (m, 1H), 1.49 (s, 6H), 1.33 (s, 3H), 0.92 (t, J=7.6 Hz, 3H).

### Example 144. Methyl ((1R,3R)-3-(6-((4-(2-hydroxypropan-2-yl)-6-(5-(2-hydroxypropan-2-yl)thiazol-2-yl)pyridin-2-yl)amino)-3-(methyl-d₃)-2-oxo-2,3-dihydro-1H-imidazo[4,5-c)pyridin-1-yl)cyclopentyl)carbamate

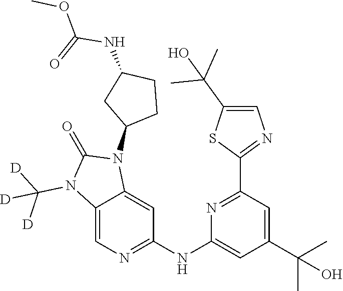 [SMILES: [2H]C([2H])([2H])n1c(=O)n([C@@H]2CC[C@@H](NC(=O)OC)C2)c2cc(Nc3cc(C(C)(C)O)cc(-c4ncc(C(C)(C)O)s4)n3)ncc21]

### Step 1. 2-(2-Bromothiazol-5-yl)propan-2-ol

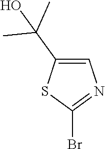

This compound was prepared according to the procedures described in Example 92, Step 4, with 1-(2-bromothiazol-5-yl)ethan-1-one replacing ethyl 2-chloro-6-(5-chlorothiazol-2-yl)isonicotinate. LCMS calculated for C₆H₉BrNOS (M+H)⁺: m/z=222.0; found 222.0.

### Step 2. 2-(2-(6-Chloro-4-(2-hydroxypropan-2-yl)pyridin-2-yl)thiazol-5-yl)propan-2-ol

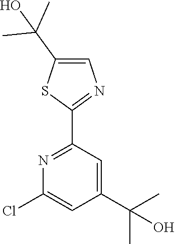 [SMILES: CC(C)(O)c1cc(Cl)nc(-c2ncc(C(C)(C)O)s2)c1]

This compound was prepared according to the procedures described in Example 92, Steps 3-4, with 2-(2-bromothiazol-5-yl)propan-2-ol replacing 2-bromo-5-chlorothiazole in Step 3. LCMS calculated for C₁₄H₁₈ClN₂O₂S (M+H)⁺: m/z=313.1; found 313.1.

### Step 3. Methyl ((1R,3R)-3-(6-((4-(2-hydroxypropan-2-yl)-6-(5-(2-hydroxypropan-2-yl)thiazol-2-yl)pyridin-2-yl)amino)-3-(methyl-d₃)-2-oxo-2,3-dihydro-1H-imidazo[4,5-c]pyridin-1-yl)cyclopentyl)carbamate

This compound was prepared according to the procedures described in Example 52, Step 5, with 2-(2-(6-chloro-4-(2-hydroxypropan-2-yl)pyridin-2-yl)thiazol-5-yl)propan-2-ol replacing 2-chloro-8-(trifluoromethyl)quinolin-4-yl)(morpholino)methanone. LCMS calculated for C₂₈H₃₃D₃N₇O₅S (M+H)⁺: m/z=585.3; found 585.3.

### Example 145. Methyl ((1R,3R)-3-(6-((6-(5-(1,1,1,3,3,3-hexafluoro-2-hydroxypropan-2-yl)thiazol-2-yl)-4-(2-hydroxypropan-2-yl)pyridin-2-yl)amino)-3-(methyl-d₃)-2-oxo-2,3-dihydro-1H-imidazo[4,5-c]pyridin-1-yl)cyclopentyl)carbamate

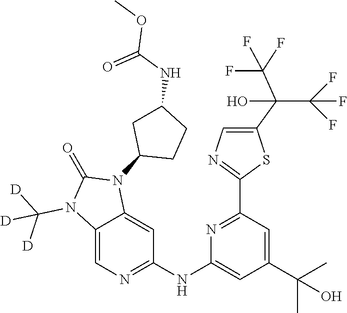 [SMILES: [2H]C([2H])([2H])n1c(=O)n([C@@H]2CC[C@@H](NC(=O)OC)C2)c2cc(Nc3cc(C(C)(C)O)cc(-c4ncc(C(O)(C(F)(F)F)C(F)(F)F)s4)n3)ncc21]

According to Example 144, with 2-(2-bromothiazol-5-yl)-1,1,1,3,3,3-hexafluoropropan-2-ol replacing 2-(2-bromothiazol-5-yl)propan-2-ol as starting materials in Step 2. LCMS calculated for C₂₈H₂₇D₃F₆N₇O₅S (M+H)⁺: m/z=693.2; found 693.2.

### Example 146. N-((1S,3R)-3-(6-((4-(2-Hydroxypropan-2-yl)-6-(2-(trifluoromethyl)thiazol-5-yl)pyridin-2-yl)amino)-3-(methyl-d₃)-2-oxo-2,3-dihydro-1H-imidazo[4,5-c]pyridin-1-yl)-1-methylcyclopentyl)acetamide

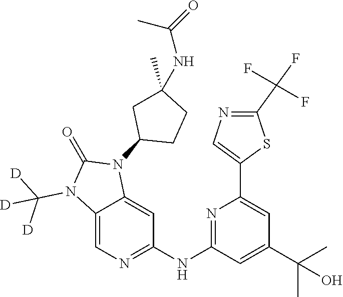 [SMILES: [2H]C([2H])([2H])n1c(=O)n([C@@H]2CC[C@](C)(NC(C)=O)C2)c2cc(Nc3cc(C(C)(C)O)cc(-c4cnc(C(F)(F)F)s4)n3)ncc21]

According to Example 151 Steps 1-5, with 5-bromo-2-(trifluoromethyl)thiazole replacing 5-bromo-2-isopropoxythiazole in Step 3. LCMS calculated for C₂₇H₂₈D₃F₃N₇O₃S (M+H)⁺: m/z=593.2; found 593.2.

### Example 147. Methyl ((1R,3R)-3-(6-((6-(5-(1,1,1,3,3,3-hexafluoro-2-hydroxypropan-2-yl)thiazol-2-yl)-4-(2-hydroxypropan-2-yl)pyridin-2-yl)amino)-3-(methyl-d₃)-2-oxo-2,3-dihydro-1H-imidazo[4,5-c]pyridin-1-yl)cyclopentyl-1-d)carbamate

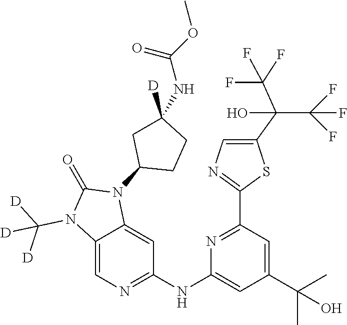 [SMILES: [2H]C([2H])([2H])n1c(=O)n([C@@H]2CC[C@@]([2H])(NC(=O)OC)C2)c2cc(Nc3cc(C(C)(C)O)cc(-c4ncc(C(O)(C(F)(F)F)C(F)(F)F)s4)n3)ncc21]

According to Example 145, with methyl ((1R,3R)-3-(6-chloro-3-(methyl-d₃)-2-oxo-2,3-dihydro-1H-imidazo[4,5-c]pyridin-1-yl)cyclopentyl-1-d)carbamate (Example 158, Step 5) as starting materials. LCMS calculated for C₂₈H₂₆D₄F₆N₇O₅S (M+H)⁺: m/z=694.2; found 694.2. ¹H NMR (500 MHZ, DMSO) δ 10.40 (s, 1H), 9.54 (s, 1H), 8.20 (s, 1H), 8.17 (s, 1H), 7.84 (s, 1H), 7.56 (s, 1H), 7.53 (s, 1H), 7.24 (s, 1H), 5.39 (s, 1H), 4.98-4.88 (m, 1H), 3.53 (s, 3H), 2.29 (dd, J=13.6, 7.2 Hz, 1H), 2.18-2.01 (m, 3H), 1.88 (dd, J=13.7, 9.6 Hz, 1H), 1.59-1.54 (m, 1H), 1.49 (s, 6H).

### Example 148. Methyl ((1R,3R)-3-(6-((6-(5-(2-hydroxypropan-2-yl)thiazol-2-yl)-4-methylpyridin-2-yl)amino)-3-(methyl-d₃)-2-oxo-2,3-dihydro-1H-imidazo[4,5-c]pyridin-1-yl)cyclopentyl-1-d)carbamate

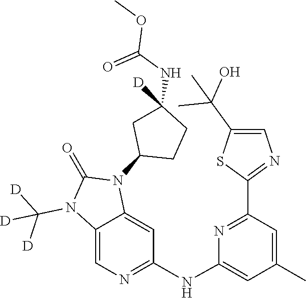 [SMILES: [2H]C([2H])([2H])n1c(=O)n([C@@H]2CC[C@@]([2H])(NC(=O)OC)C2)c2cc(Nc3cc(C)cc(-c4ncc(C(C)(C)O)s4)n3)ncc21]

According to Example 158 (Step 5), Example 91 (Step 3), Example 92 (Step 4) and Example 105 (Step 1-2), with ethyl 2-bromothiazole-5-carboxylate and 2-bromo-6-chloro-4-methylpyridine as starting materials. LCMS calculated for C₂₋₆H₂₈D₄N₇O₄S (M+H)⁺: m/z=542.2; found: 542.3.

### Example 149. Methyl ((1R,3R)-3-(6-((5-fluoro-6-(5-(2-hydroxypropan-2-yl)thiazol-2-yl)-4-methylpyridin-2-yl)amino)-3-(methyl-d₃)-2-oxo-2,3-dihydro-1H-imidazo[4,5-c]pyridin-1-yl)cyclopentyl-1-d)carbamate

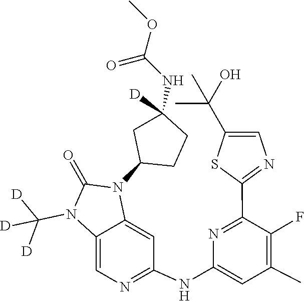 [SMILES: [2H]C([2H])([2H])n1c(=O)n([C@@H]2CC[C@@]([2H])(NC(=O)OC)C2)c2cc(Nc3cc(C)c(F)c(-c4ncc(C(C)(C)O)s4)n3)ncc21]

### Step 1. 2-(1,3-Dioxoisoindolin-2-yl)-5-fluoro-4-methylpyridine 1-oxide

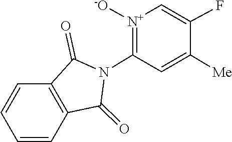

This compound was prepared according to the procedures described in WO 2020/097537 Example 11. LCMS calculated for C₁₄H₁₀FN₂O₃ (M+H)⁺: m/z=273.1; found: 273.1.

### Step 2. 2-(6-Bromo-5-fluoro-4-methylpyridin-2-yl)isoindoline-1,3-dione

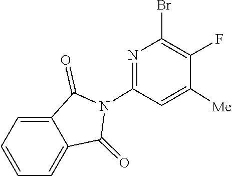 [SMILES: Cc1cc(N2C(=O)c3ccccc3C2=O)nc(Br)c1F]

A flask containing a solution of 2-(1,3-dioxoisoindolin-2-yl)-5-fluoro-4-methylpyridine 1-oxide (2.13 g, 7.81 mmol) in dichloromethane (78 mL) was cooled to 0° C. in an ice water bath and then was added phosphorus oxybromide (2.24 g, 7.81 mmol). The reaction was allowed to stir at 0° C. for 30 minutes and then allowed to slowly warm to r.t. Upon stirring for 2 h, the reaction was quenched with a saturated solution of sodium bicarbonate then extracted with ethyl acetate. The organic layer was dried over sodium sulfate, filtered, and the solvent was removed under reduced pressure. The crude product was used for the next step without purification. LCMS calculated for C₁₄H₉BrFN₂O₂ (M+H)⁺: m/z=335.0; found: 334.9.

### Step 3. 6-Bromo-5-fluoro-4-methylpyridin-2-amine

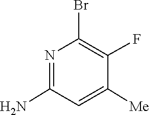

To a flask containing 2-(6-bromo-5-fluoro-4-methylpyridin-2-yl)isoindoline-1,3-dione (2.6 g, 7.8 mmol), was added a solution of ammonia (11.1 mL, 78.0 mmol, 7 N in methanol). The flask was then sealed and stirred at 45° C. for 5 h, after which the solvent was removed under reduced pressure. The crude material was purified by Biotage Isolera to give the product as a white solid. LCMS calculated for C₆H₇BrFN₂ (M+H)⁺: m/z=205.0; found: 205.0.

### Step 4. Methyl ((1R,3R)-3-(6-((5-fluoro-6-(5-(2-hydroxypropan-2-yl)thiazol-2-yl)-4-methylpyridin-2-yl)amino)-3-(methyl-d₃)-2-oxo-2,3-dihydro-1H-imidazo[4,5-c]pyridin-1-yl)cyclopentyl-1-d)carbamate

According to Example 158 (Step 5), Example 91 (Step 3), Example 92 (Step 4) and Example 105 (Step 1-2), with ethyl 2-bromothiazole-5-carboxylate and 6-bromo-5-fluoro-4-methylpyridin-2-amine as starting materials. LCMS calculated for C₂₆H₂₇D₄FN₇O₄S (M+H)⁺: m/z=560.2; found 560.2.1H NMR (500 MHz, DMSO) δ 10.47 (s, 1H), 8.19 (s, 1H), 7.85 (s, 1H), 7.59 (s, 1H), 7.28 (d, J=4.3 Hz, 1H), 7.24 (s, 1H), 5.76 (s, 1H), 4.93 (p, J=8.3 Hz, 1H), 3.53 (s, 3H), 2.38 (s, 3H), 2.30 (dd, J=13.6, 7.1 Hz, 1H), 2.15 (m, 2H), 2.14-2.01 (m, 1H), 1.92 (dd, J=13.7, 9.7 Hz, 1H), 1.60 (s, 6H), 1.56 (s, 1H).

### Example 150. Methyl ((1R,3R)-3-(6-((6-(5-(2-hydroxypropan-2-yl)thiazol-2-yl)-4-(methyl-d₃)pyridin-2-yl)amino)-3-(methyl-d₃)-2-oxo-2,3-dihydro-1H-imidazo[4,5-c]pyridin-1-yl)cyclopentyl-1-d)carbamate

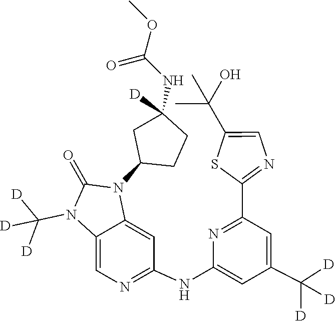 [SMILES: [2H]C([2H])([2H])c1cc(Nc2cc3c(cn2)n(C([2H])([2H])[2H])c(=O)n3[C@@H]2CC[C@@]([2H])(NC(=O)OC)C2)nc(-c2ncc(C(C)(C)O)s2)c1]

### Step 1. 6-Chloro-4-(methyl-d₃)pyridin-2-amine

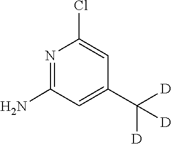 [SMILES: [2H]C([2H])([2H])c1cc(N)nc(Cl)c1]

To a solution of zinc iodide (1.26 g, 3.95 mmol) in THF (2.6 mL) at 0° C. was added (methyl-d₃)magnesium bromide (3.95 mL, 3.95 mmol, IM in diethyl ether). To the formed slurry was then added 4-bromo-6-chloropyridin-2-amine (164 mg, 0.8 mmol) in THF (2.6 mL), followed by bis(triphenylphosphine)palladium(II) dichloride (56 mg, 79 μmol). The reaction mixture was stirred at 50° C. for 3h. MeOH was added to quench the reaction. The reaction mixture was then diluted with ethyl acetate, and the organic layer was subsequently washed with water and brine, dried over sodium sulfate, filtered, and concentrated in vacuo. The crude material was purified by Biotage Isolera to give the product as a white solid. LCMS calculated for C₆H₅D₃ClN₂ (M+H)⁺: m/z=146.0; found 146.1.

### Step 2. Methyl ((1R,3R)-3-(6-((6-(5-(2-hydroxypropan-2-yl)thiazol-2-yl)-4-(methyl-d₃)pyridin-2-yl)amino)-3-(methyl-d₃)-2-oxo-2,3-dihydro-1H-imidazo[4,5-c]pyridin-1-yl)cyclopentyl-1-d)carbamate

According to Example 158 (Step 5), Example 91 (Step 3), Example 92 (Step 4) and Example 105 (Step 1-2), With ethyl 2-bromothiazole-5-carboxylate and 6-chloro-4-(methyl-d₃)pyridin-2-amine as starting materials. LCMS calculated for C₂₆H₂₅D₇N₇O₄S (M+H)⁺: m/z=545.3; found 545.3.1H NMR (600 MHz, DMSO) δ 10.46 (s, 1H), 8.22 (s, 1H), 7.78 (s, 1H), 7.69 (s, 1H), 7.56 (s, 1H), 7.23 (s, 1H), 7.14 (s, 1H), 5.75 (s, 1H), 4.99-4.90 (m, 1H), 3.53 (s, 3H), 2.32 (dd, J=13.7, 7.0 Hz, 1H), 2.21-2.11 (m, 2H), 2.13-2.05 (m, 1H), 1.93 (dd, J=13.7, 9.7 Hz, 1H), 1.60 (s, 6H), 1.57 (m, 1H).

### Example 151. N-((1S,3R)-3-(6-((4-(2-Hydroxypropan-2-yl)-6-(2-isopropoxythiazol-5-yl)pyridin-2-yl)amino)-3-(methyl-d₃)-2-oxo-2,3-dihydro-1H-imidazo[4,5-c]pyridin-1-yl)-1-methylcyclopentyl)acetamide

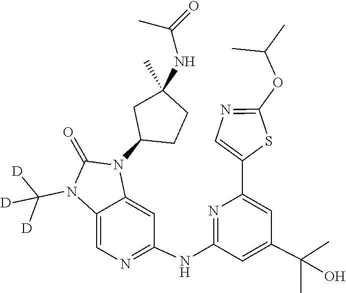 [SMILES: [2H]C([2H])([2H])n1c(=O)n([C@@H]2CC[C@](C)(NC(C)=O)C2)c2cc(Nc3cc(C(C)(C)O)cc(-c4cnc(OC(C)C)s4)n3)ncc21]

### Step 1. N-((1S,3R)-3-(6-Amino-3-(methyl-d₃)-2-oxo-2,3-dihydro-1H-imidazo[4,5-c]pyridin-1-yl)-1-methylcyclopentyl)acetamide

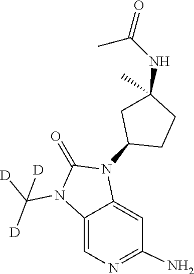 [SMILES: [2H]C([2H])([2H])n1c(=O)n([C@@H]2CC[C@](C)(NC(C)=O)C2)c2cc(N)ncc21]

This compound was prepared according to the procedures described in Intermediate A and Example 52 Step 4, with tert-butyl ((1S,3R)-3-amino-1-methylcyclopentyl)carbamate replacing tert-butyl ((1R,3R)-3-aminocyclobutyl)carbamate in Step 1 of Intermediate A, and with acetyl chloride replacing methyl carbonochloridate in Step 5 of Intermediate A. LCMS calculated for C₁₅H₁₉D₃N₅O₂ (M+H)⁺: m/z=307.2; found: 307.2.

### Step 2. Methyl 2-amino-6-(tributylstannyl)isonicotinate

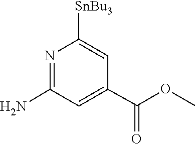

A reaction vial containing methyl 2-amino-6-chloroisonicotinate (1.0 g, 5.36 mmol), 1,1,1,2,2,2-hexabutyldistannane (4.1 mL, 8.0 mmol), tricyclohexylphosphane (0.3 g, 1.1 mmol), lithium chloride (0.23 g, 5.4 mmol) and Pd₂(dba)₃ (0.49 g, 0.54 mmol) was evacuated and backfilled with nitrogen three times. Then 1,4-dioxane (27 ml) was added. The reaction mixture was heated to 125° C. for 5h. The mixture was cooled down to r.t., was concentrated in vacuo and purified by Biotage Isolera to give the product as an orange solid. LCMS calculated for C₁₉H₃N₂O₂Sn (M+H)⁺: m/z=443.2; found 443.2.

### Step 3. Methyl 2-amino-6-(2-isopropoxythiazol-5-yl)isonicotinate

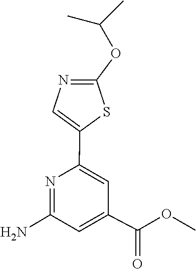 [SMILES: COC(=O)c1cc(N)nc(-c2cnc(OC(C)C)s2)c1]

A reaction vial containing a mixture of methyl 2-amino-6-(tributylstannyl)isonicotinate (150 mg, 0.34 mmol), 5-bromo-2-isopropoxythiazole (50 mg, 0.23 mmol), copper(I) iodide (21 mg, 0.11 mmol), and tetrakis(triphenylphosphine)palladium(0) (52 mg, 45.0 μmol) was evacuated and backfilled with nitrogen, followed by the addition of 1,4-dioxane (2.2 mL). After stirring at 60° C. for 3 hours, the reaction mixture was cooled to r.t. and was diluted with ethyl acetate. The organic layer was subsequently washed with water and brine, dried over sodium sulfate, filtered, and concentrated in vacuo. The crude material was purified by Biotage Isolera to give the product as a light-yellow residue. LCMS calculated for C₁₃H₁₆N₃O₃S (M+H)⁺: m/z=294.1; found 294.1.

### Step 4. 2-(2-Amino-6-(2-isopropoxythiazol-5-yl)pyridin-4-yl)propan-2-ol

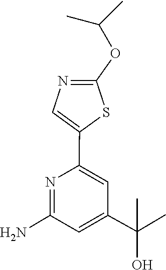 [SMILES: CC(C)Oc1ncc(-c2cc(C(C)(C)O)cc(N)n2)s1]

This compound was prepared according to the procedures described in Example 92, Step 2, with methyl 2-amino-6-(2-isopropoxythiazol-5-yl)isonicotinate replacing ethyl 2-chloro-6-(5-chlorothiazol-2-yl)isonicotinate. LCMS calculated for C₁₄H₂₀N₃O₂S (M+H)⁺: m/z=294.1; found 294.1.

### Step 5. N-((1S,3R)-3-(6-((4-(2-hydroxypropan-2-yl)-6-(2-isopropoxythiazol-5-yl)pyridin-2-yl)amino)-3-(methyl-d₃)-2-oxo-2,3-dihydro-1H-imidazo[4,5-c]pyridin-1-yl)-1-methylcyclopentyl)acetamide

This compound was prepared according to the procedures described in Example 52, Step 5, with N-((1S,3R)-3-(6-amino-3-(methyl-d₃)-2-oxo-2,3-dihydro-1H-imidazo[4,5-c]pyridin-1-yl)-1-methylcyclopentyl)acetamide replacing methyl ((1R,3R)-3-(6-amino-3-(methyl-d₃)-2-oxo-2,3-dihydro-1H-imidazo[4,5-c]pyridin-1-yl)cyclopentyl)carbamate and with 2-(2-amino-6-(2-isopropoxythiazol-5-yl)pyridin-4-yl)propan-2-ol replacing (2-chloro-8-(trifluoromethyl)quinolin-4-yl)(morpholino)methanone. LCMS calculated for C₂₉H₃₅D₃N₇O₄S (M+H)⁺: m/z=583.3; found 583.3.

### Example 152. N-((1S,3R)-3-(6-((4-(2-Hydroxypropan-2-yl)-6-(2-methoxythiazol-5-yl)pyridin-2-yl)amino)-3-(methyl-d₃)-2-oxo-2,3-dihydro-1H-imidazo[4,5-c]pyridin-1-yl)-1-methylcyclopentyl)acetamide

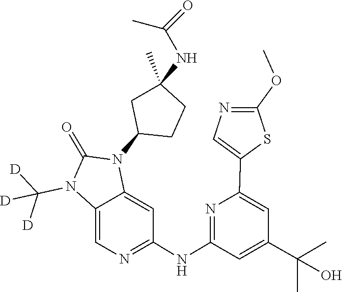 [SMILES: [2H]C([2H])([2H])n1c(=O)n([C@@H]2CC[C@](C)(NC(C)=O)C2)c2cc(Nc3cc(C(C)(C)O)cc(-c4cnc(OC)s4)n3)ncc21]

According to Example 151 Steps 1-5, with 5-bromo-2-methoxythiazole replacing 5-bromo-2-isopropoxythiazole in Step 3. LCMS calculated for C₂₇H₃D₃N₇O₄S (M+H)⁺: m/z=555.3; found 555.3.

### Example 153. N-((1S,3R)-3-(6-((6-(2-Ethoxythiazol-5-yl)-4-(2-hydroxypropan-2-yl)pyridin-2-yl)amino)-3-(methyl-d₃)-2-oxo-2,3-dihydro-1H-imidazo[4,5-c]pyridin-1-yl)-1-methylcyclopentyl)acetamide

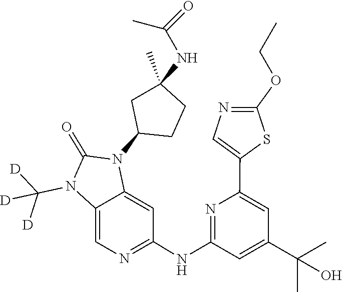 [SMILES: [2H]C([2H])([2H])n1c(=O)n([C@@H]2CC[C@](C)(NC(C)=O)C2)c2cc(Nc3cc(C(C)(C)O)cc(-c4cnc(OCC)s4)n3)ncc21]

According to Example 151 Steps 1-5, with 5-bromo-2-ethoxythiazole replacing 5-bromo-2-isopropoxythiazole in Step 3. LCMS calculated for C₂₈H₃₃D₃N₇O₄S (M+H)⁺: m/z=569.3; found 569.3.

### Example 154. Methyl ((1R,3R)-3-(6-((6′-(5-(difluoromethyl)thiophen-2-yl)-4-(trifluoromethyl)-[3,4′-bipyridin]-2′-yl)amino)-3-(methyl-d₃)-2-oxo-2,3-dihydro-1H-imidazo[4,5-c]pyridin-1-yl)cyclopentyl)carbamate

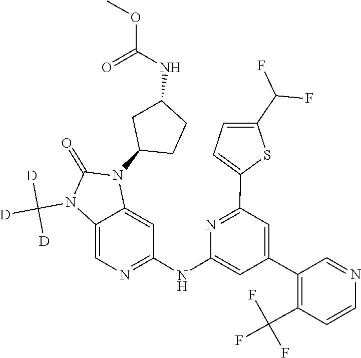 [SMILES: [2H]C([2H])([2H])n1c(=O)n([C@@H]2CC[C@@H](NC(=O)OC)C2)c2cc(Nc3cc(-c4cnccc4C(F)(F)F)cc(-c4ccc(C(F)F)s4)n3)ncc21]

### Step 1. 4-Chloro-6-(5-(difluoromethyl)thiophen-2-yl)pyridin-2-amine

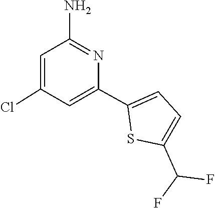 [SMILES: Nc1cc(Cl)cc(-c2ccc(C(F)F)s2)n1]

To a vial containing 6-bromo-4-chloropyridin-2-amine (100 mg, 0.48 mmol) in dioxane (2 mL) and water (0.2 mL) was added 2-(5-(difluoromethyl)thiophen-2-yl)-4,4,5,5-tetramethyl-1,3,2-dioxaborolane (125 mg, 0.48 mmol), Pd(dppf)₂Cl₂ dichloromethane adduct (39 mg, 0.048 mmol) and sodium carbonate (102 mg, 0.96 mmol) under N₂ atmosphere. The reaction was stirred at 80° C. for 1 h. The mixture was cooled down to r.t., the solids were filtered off, and the solvent was evaporated in vacuo. The crude material was purified by Biotage Isolera to give the product. LCMS calculated for C₁₀H₈ClF₂N₂S (M+H)⁺: m/z=261.0; found: 261.0.

### Step 2. 6′-(5-(Difluoromethyl)thiophen-2-yl)-4-(trifluoromethyl)-[3,4′-bipyridin]-2′-amine

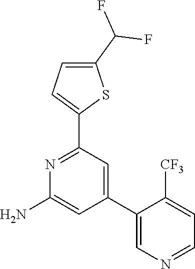 [SMILES: Nc1cc(-c2cnccc2C(F)(F)F)cc(-c2ccc(C(F)F)s2)n1]

To a vial containing 4-chloro-6-(5-(difluoromethyl)thiophen-2-yl)pyridin-2-amine (40 mg, 0.15 mmol) in dioxane (2 mL) and water (0.1 mL) was added 3-(4,4,5,5-tetramethyl-1,3,2-dioxaborolan-2-yl)-4-(trifluoromethyl)pyridine (84 mg, 0.31 mmol), XPhos Pd G2 (24 mg, 0.03 mmol) and cesium carbonate (150 mg, 0.46 mmol) under N₂ atmosphere. The reaction was stirred at 80° C. for 1 h. The mixture was cooled down to r.t., the solids were filtered off, and the solvent was evaporated in vacuo. The crude material was purified by Biotage Isolera to give the product. LCMS calculated for C₁₆H₁₁FN₃S (M+H)⁺: m/z=372.1; found: 372.1.

### Step 3. Methyl (1R,3R)-3-(6-(6′-(5-(difluoromethyl)thiophen-2-yl)-4-(trifluoromethyl)-[3,4′-bipyridin]-2′-yl)amino)-3-(methyl-d₃)-2-oxo-2,3-dihydro-1H-imidazo[4,5-c]pyridin-1-yl)cyclopentyl)carbamate

This compound was prepared according to the procedures described in Example 92, with 6′-(5-(difluoromethyl)thiophen-2-yl)-4-(trifluoromethyl)-[3,4′-bipyridin]-2′-amine replacing 2-(2-amino-6-(5-chlorothiazol-2-yl)pyridin-4-yl)propan-2-ol in Step 6. LCMS calculated for C₃₀H₂₄D₃F₅N₇O₃S (M+H)⁺: m/z=663.2; found: 663.2.

### Example 155. Methyl ((1S,2S,4S)-2-hydroxy-4-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl)carbamate

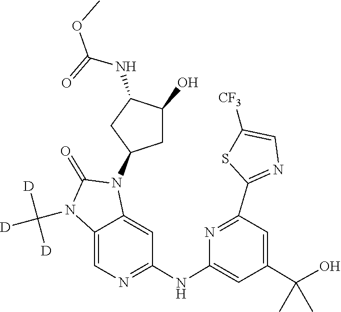 [SMILES: [2H]C([2H])([2H])n1c(=O)n([C@H]2C[C@H](NC(=O)OC)[C@@H](O)C2)c2cc(Nc3cc(C(C)(C)O)cc(-c4ncc(C(F)(F)F)s4)n3)ncc21]

### Step 1. tert-Butyl ((1R,3r,5S)-6-oxabicyclo[3.1.0]hexan-3-yl)carbamate

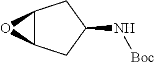 [SMILES: CC(C)(C)OC(=O)N[C@@H]1C[C@@H]2O[C@@H]2C1]

To a flask containing tert-butyl cyclopent-3-en-1-ylcarbamate (4.1 g, 22.2 mmol) in DCM (100 mL) at 0° C. was added sodium bicarbonate (3.7 g, 44.4 mmol) and mCPBA (6.0 g, 26.7 mmol) portion wise. The mixture was stirred at r.t. for 48 h. The mixture was quenched with sat. NaHSO₃ solution and extracted with DCM. The organic layers were combined, washed with sat. NaHCO₃ solution, dried over sodium sulfate, filtered, and the solvents were evaporated in vacuo. The crude material was purified by Biotage Isolera to give the product. LCMS calculated for C₁₀H₁₈NO₃ (M+H)⁺: m/z=200.1; found: 200.1.

### Step 2. tert-Butyl ((1S,3S,4S)-3-azido-4-hydroxycyclopentyl)carbamate

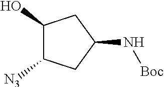 [SMILES: CC(C)(C)OC(=O)N[C@H]1C[C@H](N=[N+]=[N-])[C@@H](O)C1]

To a flask containing tert-butyl ((1R,3r,5S)-6-oxabicyclo[3.1.0]hexan-3-yl)carbamate (4.4 g, 22.0 mmol) in 2-methoxyethanol (30 mL) and water (6 mL) was added ammonium chloride (0.58 g, 11.0 mmol) and sodium azide (4.6 g, 55.0 mmol). The mixture was stirred at 80° C. for 4 h. The mixture was concentrated to dryness. The residue was dissolved in DCM and the organic layer was washed with sat. NaHCO₃ solution, dried over sodium sulfate, filtered, and the solvents were evaporated in vacuo. The crude material was purified by Biotage Isolera to give the product. LCMS calculated for C₁₀H₁₉N₄O₃ (M+H)⁺: m/z=243.1; found: 243.1.

### Step 3. (1S,2S,4S)-2-Azido-4-((2-chloro-5-nitropyridin-4-yl)amino)cyclopentan-1-ol

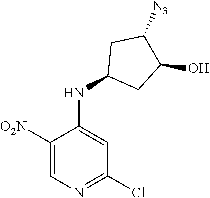 [SMILES: [N-]=[N+]=N[C@H]1C[C@H](Nc2cc(Cl)ncc2[N+](=O)[O-])C[C@@H]1O]

To a flask containing tert-butyl ((1S,3S,4S)-3-azido-4-hydroxycyclopentyl)carbamate (1.5 g, 6.2 mmol) was added 4M HCl in dioxane (10 mL, 40.0 mmol). The mixture stirred at 40° C. for 1 h. The solvents were evaporated in vacuo. To the resulting residue was added 2,4-dichloro-5-nitropyridine (1.2 g, 6.19 mmol) and N-ethyl-N-isopropylpropan-2-amine (3.3 mL, 18.6 mmol) in acetonitrile (12 mL). The mixture was stirred at 55° C. for 1 h. The mixture was concentrated to dryness. The residue was dissolved in EtOAc and the organic layer was washed with sat. NaHCO₃ solution, dried over sodium sulfate, filtered, and the solvents were evaporated in vacuo. The crude material was purified by Biotage Isolera to give the product. LCMS calculated for C₁₀H₁₂ClN₆O₃ (M+H)⁺: m/z=299.1; found: 299.1.

### Step 4. (1S,2S,4S)-2-Amino-4-((2-chloro-5-nitropyridin-4-yl)amino)cyclopentan-1-ol

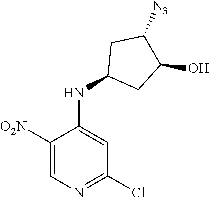 [SMILES: [N-]=[N+]=N[C@H]1C[C@H](Nc2cc(Cl)ncc2[N+](=O)[O-])C[C@@H]1O]

To a flask containing (1S,2S,4S)-2-azido-4-((2-chloro-5-nitropyridin-4-yl)amino)cyclopentan-1-ol (1.85 g, 6.19 mmol) in THF (20 mL) and water (2.2 mL) was added polymer supported triphenylphosphine (4.9 g, 9.3 mmol). The mixture was stirred at 40° C. for 3 h. The reaction mixture was filtered through sodium sulfate pad and the solvents were evaporated in vacuo. The crude was used for the next step without further purification. LCMS calculated for C₁₀H₁₄ClN₄O₃ (M+H)⁺: m/z=273.1; found: 273.1.

### Step 5. tert-Butyl ((1S,2S,4S)-4-((2-chloro-5-nitropyridin-4-yl)amino)-2-hydroxycyclopentyl)-carbamate

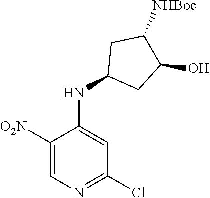 [SMILES: CC(C)(C)OC(=O)N[C@H]1C[C@H](Nc2cc(Cl)ncc2[N+](=O)[O-])C[C@@H]1O]

To a flask containing (1S,2S,4S)-2-amino-4-((2-chloro-5-nitropyridin-4-yl)amino)cyclopentan-1-ol (1.68 g, 6.16 mmol) in MeOH (20 mL) was added di-tert-butyl dicarbonate (2.69 g, 12.3 mmol). The mixture was stirred at r.t. for 15 min and the solvents were evaporated in vacuo. The crude material was purified by Biotage Isolera to give the product. LCMS calculated for C₁₅H₂₂ClN₄O₅ (M+H)⁺: m/z=373.1; found: 373.1.

### Step 6. tert-Butyl ((1S,2S,4S)-4-(6-chloro-3-(methyl-d₃)-2-oxo-2,3-dihydro-1H-imidazo[4,5-c]pyridin-1-yl)-2-hydroxycyclopentyl)carbamate

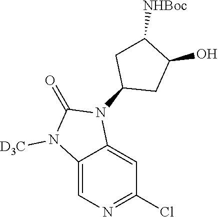 [SMILES: [2H]C([2H])([2H])n1c(=O)n([C@H]2C[C@H](NC(=O)OC(C)(C)C)[C@@H](O)C2)c2cc(Cl)ncc21]

This compound was prepared according to the procedures described in Intermediate A, with tert-butyl ((1S,2S,4S)-4-((2-chloro-5-nitropyridin-4-yl)amino)-2-hydroxycyclopentyl)-carbamate replacing tert-butyl ((1R,3R)-3-((2-chloro-5-nitropyridin-4-yl)amino)cyclopentyl)carbamate in Step 2. LCMS calculated for C₁₇H₂₁D₃ClN₄O₄ (M+H)⁺: m/z=386.2; found: 386.2.

### Step 7. tert-Butyl ((1S,2S,4S)-2-hydroxy-4-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl)carbamate

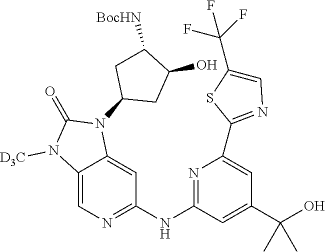 [SMILES: [2H]C([2H])([2H])n1c(=O)n([C@H]2C[C@H](NC(=O)OC(C)(C)C)[C@@H](O)C2)c2cc(Nc3cc(C(C)(C)O)cc(-c4ncc(C(F)(F)F)s4)n3)ncc21]

This compound was prepared according to the procedures described in Example 92, with tert-butyl ((1S,2S,4S)-4-(6-chloro-3-(methyl-d₃)-2-oxo-2,3-dihydro-1H-imidazo[4,5-c]pyridin-1-yl)-2-hydroxycyclopentyl)carbamate replacing N-((1S,3R)-3-(6-chloro-3-(methyl-d₃)-2-oxo-2,3-dihydro-1H-imidazo[4,5-c]pyridin-1-yl)-1-methylcyclopentyl)cyclopropanecarboxamide and 2-(2-amino-6-(5-(trifluoromethyl)thiazol-2-yl)pyridin-4-yl)propan-2-ol replacing 2-(2-amino-6-(5-chlorothiazol-2-yl)pyridin-4-yl)propan-2-ol in Step 6. LCMS calculated for C₂₉H₃₂D₃F₃N₇O₅S (M+H)⁺: m/z=653.3; found: 653.3.

### Step 8. Methyl ((1S,2S,4S)-2-hydroxy-4-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl)carbamate

To a vial containing tert-butyl ((1S,2S,4S)-2-hydroxy-4-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl)carbamate (30 mg, 0.046 mmol) was added 4M HCl in dioxane (1 mL, 4.0 mmol). The mixture stirred at r.t. for 10 min. and the solvents were evaporated in vacuo. To the residue was added DIPEA (16 μl, 0.092 mmol), MeOH (0.05 mL) and DCM (0.5 mL). To the resulting mixture was added methyl carbonochloridate (5 μl, 0.069 mmol). The reaction was stirred at r.t. for 20 min. and the product was purified by prep-LCMS (XBridge C18 column, eluting with a gradient of acetonitrile/water containing 0.1% TFA, at flow rate of 60 mL/min). LCMS calculated for C₂₆H₂₆D₃F₃N₇O₅S (M+H)⁺: m/z=611.2; found: 611.2.

### Example 156. Methyl ((1R,3R)-3-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl-3-d)carbamate

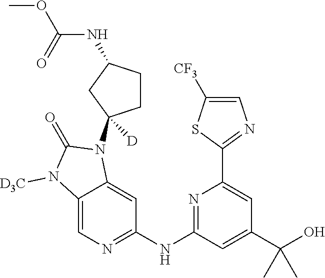 [SMILES: [2H]C([2H])([2H])n1c(=O)n([C@]2([2H])CC[C@@H](NC(=O)OC)C2)c2cc(Nc3cc(C(C)(C)O)cc(-c4ncc(C(F)(F)F)s4)n3)ncc21]

According to Example 97 (Step 6-7), and Example 137 (Step 3). LCMS calculated for C₂₆H₂₅D₄F₃N₇O₄S (M+H)⁺: m/z=596.2; found 596.2.

### Example 157. Methyl ((1R,3R)-3-(3-(methyl-d₃)-6-((4-(1-methylpiperidin-4-yl)-6-(5-(trifluoromethyl)thiazol-2-yl)pyridin-2-yl)amino)-2-oxo-2,3-dihydro-1H-imidazo[4,5-c]pyridin-1-yl)cyclopentyl)carbamate

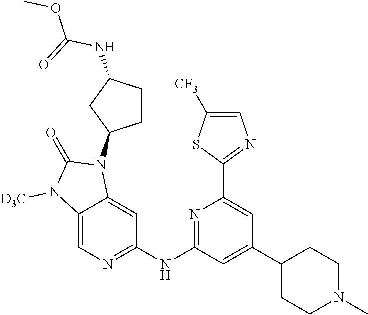 [SMILES: [2H]C([2H])([2H])n1c(=O)n([C@@H]2CC[C@@H](NC(=O)OC)C2)c2cc(Nc3cc(C4CCN(C)CC4)cc(-c4ncc(C(F)(F)F)s4)n3)ncc21]

According to Example 105 (Step 4) and Example 97 (Step 5). LCMS calculated for C₂₉H₃₁D₃F₃N₈O₃S (M+H)⁺: m/z=634.2; found 634.2.

### Example 158. Methyl ((1R,3R)-3-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl-1-d)carbamate

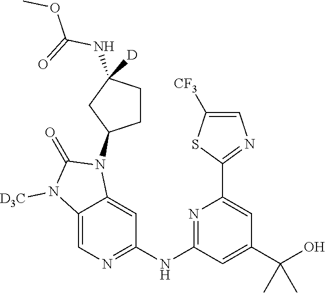 [SMILES: [2H]C([2H])([2H])n1c(=O)n([C@@H]2CC[C@@]([2H])(NC(=O)OC)C2)c2cc(Nc3cc(C(C)(C)O)cc(-c4ncc(C(F)(F)F)s4)n3)ncc21]

### Step 1. tert-Butyl ((1R,3S)-3-hydroxycyclopentyl-3-d)carbamate

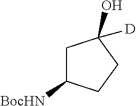 [SMILES: [2H][C@]1(O)CC[C@@H](NC(=O)OC(C)(C)C)C1]

To a flask containing tert-butyl (R)-(3-oxocyclopentyl)carbamate (10 g, 50.2 mmol) in MeOH (100 mL) at 0° C. was added sodium borodeuteride-d₄ (1.26 g, 30.1 mmol) in portions (over 10 times) during 30 min. The reaction mixture was stirred at 0° C. for 3 h. The reaction mixture was concentrated, diluted with CH₂Cl₂ (250 mL), and washed with 1.0 M sodium hydroxide solution. After extraction with CH₂Cl₂ (100 mL) for 3 times, the organic layers were combined, dried over MgSO₄ and concentrated in vacuo. The crude material was purified by CombiFlash to give the desired product tert-butyl ((1R,3S)-3-hydroxycyclopentyl-3-d)carbamate (earlier peak) as a white solid. The other diastereomer (later peak) was also collected to give tert-butyl ((1R,3R)-3-hydroxycyclopentyl-3-d)carbamate and used in Example 159. LCMS calculated for C₆H₁₁DNO₃ (M+H-t-Bu)⁺: m/z=147.1; found 147.1.

### Step 2. tert-Butyl ((1R,3R)-3-(1,3-dioxoisoindolin-2-yl)cyclopentyl-3-d)carbamate

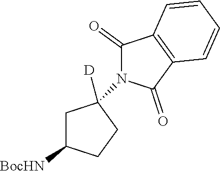 [SMILES: [2H][C@@]1(N2C(=O)c3ccccc3C2=O)CC[C@@H](NC(=O)OC(C)(C)C)C1]

To a flask containing tert-butyl ((1R,3S)-3-hydroxycyclopentyl-3-d)carbamate (4.0 g, 19.7 mmol) in THF (80 mL) was added triphenylphosphine (6.2 g, 23.6 mmol) and isoindoline-1,3-dione (4.3 g, 29.5 mmol). The reaction mixture was cooled down to 0° C. and the diisopropyl (E)-diazene-1,2-dicarboxylate (4.8 g, 4.6 mL, 23.6 mmol) was added dropwise to the flask. The reaction mixture was stirred at 0° C. to rt for 3 h. The reaction mixture was quenched with water (30 mL) and extraction with CH₂Cl₂. The organic layer was combined, dried over MgSO₄ and concentrated in vacuo. The crude material was purified by Biotage Isolera. LCMS calculated for C₁₈H₂₂DN₂O₄ (M+H)⁺: m/z=332.2; found 332.1.

### Step 3. tert-Butyl ((1R,3R)-3-aminocyclopentyl-3-d)carbamate

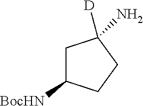 [SMILES: [2H][C@@]1(N)CC[C@@H](NC(=O)OC(C)(C)C)C1]

To a flask containing tert-butyl ((1R, 3R)-3-(1,3-dioxoisoindolin-2-yl)cyclopentyl-3-d)carbamate (5.5 g, 16.6 mmol) in ethanol (55 mL) was added hydrazine (1.1 g, 1.0 mL, 33.2 mmol). The reaction mixture was stirred at 80° C. for 1.5 h with white solid generation during the reaction. After cooling down to r.t., the reaction mixture was filtered and washed with ethanol (100 mL) and CH₂Cl₂ (100 mL) to filter out the white solid as side product. The filtrate was collected and concentrated in vacuo to give crude product. The obtained crude product was used in the next step without further purification. LCMS calculated for C₆H₁₂DN₂O₂ (M+H-t-Bu)⁺: m/z=146.1; found: 146.1.

### Step 4. Methyl ((1R,3R)-3-aminocyclopentyl-1-d)carbamate

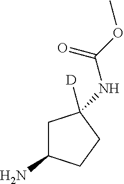

To a flask containing tert-butyl ((1R, 3R)-3-aminocyclopentyl-3-d)carbamate (5.0 g, 24.8 mmol) and DIPEA (3.5 g, 4.8 mL, 27.3 mmol) in DCM (200 mL) and MeOH (5 mL) at 0° C. was added methyl chloroformate (1.9 mL, 24.8 mmol) dropwise. After stirring at 0° C. for 10 min., the reaction was quenched with MeOH (10 mL) and concentrated in vacuo. The residue was re-dissolved in CH₂Cl₂ (200 mL), was washed with sodium bicarbonate saturated solution, dried over MgSO₄ and concentrated in vacuo. The crude material was treated with 4.0 M HCl in dioxane (20 mL). After stirring at r.t. for 1 h, the reaction mixture was concentrated in vacuo. The obtained crude product was used in the next step without further purification. LCMS calculated for C₇H₁₄DN₂O₂ (M+H)⁺: m/z=160.1; found: 160.1.

### Step 5. Methyl ((1R,3R)-3-(6-chloro-3-(methyl-d₃)-2-oxo-2,3-dihydro-1H-imidazo[4,5-c]pyridin-1-yl)cyclopentyl-1-d)carbamate

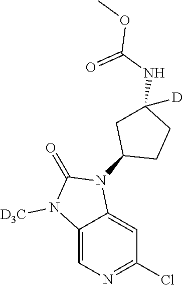 [SMILES: [2H]C([2H])([2H])n1c(=O)n([C@@H]2CC[C@@]([2H])(NC(=O)OC)C2)c2cc(Cl)ncc21]

The title compound was prepared according to the procedures described for Intermediate A, using methyl ((1R,3R)-3-aminocyclopentyl-1-d)carbamate as starting material. LCMS calculated for C₁₄H₁₄D₄ClN₄O₃ (M+H)⁺: m/z=329.1; found: 329.1.

### Step 6. Methyl ((1R,3R)-3-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl-1-d)carbamate

The title compound was prepared according to the procedures described for Example 137, with methyl ((1R,3R)-3-(6-chloro-3-(methyl-d₃)-2-oxo-2,3-dihydro-1H-imidazo[4,5-c]pyridin-1-yl)cyclopentyl-1-d)carbamate as starting material in Step 5. LCMS calculated for C₂₆H₂₅D₄F₃N₇O₄S (M+H)⁺: m/z=596.2; found 596.2. ¹H NMR (600 MHz, DMSO) δ 10.74 (s, 1H), 8.61 (d, J=1.5 Hz, 1H), 8.31 (s, 1H), 7.91 (d, J=1.3 Hz, 1H), 7.61 (s, 1H), 7.50 (s, 1H), 7.29 (s, 1H), 4.96 (p, J=8.5 Hz, 1H), 3.52 (s, 3H), 2.34 (dd, J=13.6, 7.6 Hz, 1H), 2.19-2.02 (m, 3H), 1.88 (dd, J=13.7, 9.5 Hz, 1H), 1.62-1.53 (m, 1H), 1.50 (s, 6H).

### Example 159. Methyl ((1S,3R)-3-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl-1-d)carbamate

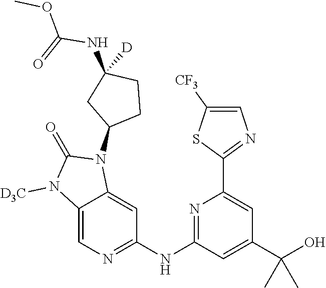 [SMILES: [2H]C([2H])([2H])n1c(=O)n([C@@H]2CC[C@]([2H])(NC(=O)OC)C2)c2cc(Nc3cc(C(C)(C)O)cc(-c4ncc(C(F)(F)F)s4)n3)ncc21]

According to Example 158, with the later peak isolated in Step 1. LCMS calculated for C₂₋₆H₂₅D₄F₃N₇O₄S (M+H)⁺: m/z=596.2; found 596.2.

### Example 160. Methyl ((1S,3R)-3-(6-((3-(1-(difluoromethyl)-1H-pyrazol-4-yl)-2-methyl-8-(4-(trifluoromethyl)pyridin-3-yl)imidazo[1,2-b]pyridazin-6-yl)amino)-3-(methyl-d₃)-2-oxo-2,3-dihydro-1H-imidazo[4,5-c]pyridin-1-yl)cyclopentyl-1-d)carbamate

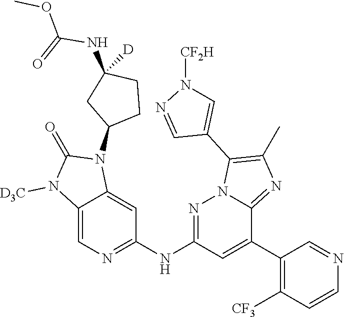 [SMILES: [2H]C([2H])([2H])n1c(=O)n([C@@H]2CC[C@]([2H])(NC(=O)OC)C2)c2cc(Nc3cc(-c4cnccc4C(F)(F)F)c4nc(C)c(-c5cnn(C(F)F)c5)n4n3)ncc21]

According to Example 97 (Step 4) and Example 159. LCMS calculated for C₃₁H₂₅D₄F₅N₁₁O₃ (M+H)⁺: m/z=702.3; found: 702.3. ¹H NMR (600 MHZ, DMSO) δ 10.44 (s, 1H), 9.07 (d, J=5.2 Hz, 1H), 8.95 (s, 1H), 8.83 (s, 1H), 8.58 (s, 1H), 8.22 (s, 1H), 8.04 (d, J=5.2 Hz, 1H), 7.94 (t, ²J_{H-F}=58.9 Hz, 1H), 7.76 (s, 1H), 7.39 (d, J=16.2 Hz, 2H), 4.65 (p, J=8.8 Hz, 1H), 3.52 (s, 3H), 2.49 (s, 3H), 2.25 (dd, J=12.8, 8.7 Hz, 1H), 2.16-1.97 (m, 3H), 1.90 (m, 1H), 1.77-1.71 (m, 1H).

### Example 161. Methyl ((1R,3R)-3-(6-((6-(5-(2-hydroxypropan-2-yl)thiazol-2-yl)-4-(tetrahydro-2H-pyran-4-yl)pyridin-2-yl)amino)-3-(methyl-d₃)-2-oxo-2,3-dihydro-1H-imidazo[4,5-c]pyridin-1-yl)cyclopentyl)carbamate

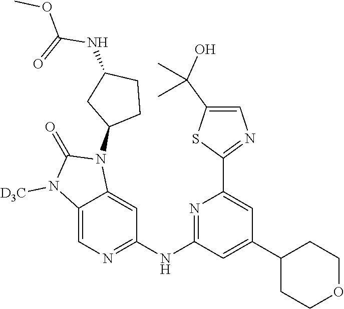 [SMILES: [2H]C([2H])([2H])n1c(=O)n([C@@H]2CC[C@@H](NC(=O)OC)C2)c2cc(Nc3cc(C4CCOCC4)cc(-c4ncc(C(C)(C)O)s4)n3)ncc21]

According to Intermediate A, Example 92 (Step 4), and Example 105 (Step 1-4), with ethyl 2-bromothiazole-5-carboxylate and 1-methyl-4-(4,4,5,5-tetramethyl-1,3,2-dioxaborolan-2-yl)-1H-pyrazole as starting materials. LCMS calculated for C₃₀H₃₅D₃N₇O₅S (M+H)⁺: m/z=611.3; found: 611.3.

### Example 162. Methyl ((1R,3R)-3-(6-((6-(5-(2-hydroxypropan-2-yl)thiazol-2-yl)-4-(tetrahydro-2H-pyran-4-yl)pyridin-2-yl)amino)-3-(methyl-d₃)-2-oxo-2,3-dihydro-1H-imidazo[4,5-c]pyridin-1-yl)cyclopentyl-1-d)carbamate

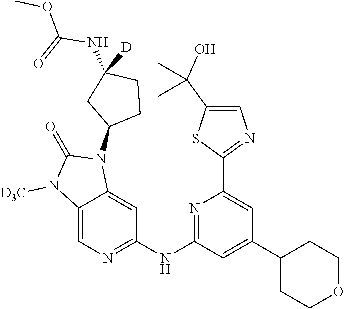 [SMILES: [2H]C([2H])([2H])n1c(=O)n([C@@H]2CC[C@@]([2H])(NC(=O)OC)C2)c2cc(Nc3cc(C4CCOCC4)cc(-c4ncc(C(C)(C)O)s4)n3)ncc21]

According to Example 158 (Step 5), and Example 161. LCMS calculated for C₃₀H₃₄D₄N₇O₅S (M+H)⁺: m/z=612.3; found: 612.3. ¹H NMR (600 MHz, DMSO) δ 10.72 (s, 1H), 8.27 (s, 1H), 7.79 (s, 1H), 7.67-7.61 (m, 2H), 7.25 (s, 1H), 7.15 (s, 1H), 4.95 (p, J=8.4 Hz, 1H), 3.99 (dd, J=11.0, 3.9 Hz, 2H), 3.53 (s, 3H), 3.51-3.42 (m, 2H), 2.93 (m, 1H), 2.32 (dd, J=13.7, 7.2 Hz, 1H), 2.21-2.04 (m, 3H), 1.93 (dd, J=13.7, 9.7 Hz, 1H), 1.83-1.77 (m, 2H), 1.74-1.64 (m, 2H), 1.63-1.58 (m, 1H), 1.60 (s, 6H).

### Example 163. Methyl ((1S,3R)-3-(6-((6-(5-(2-hydroxypropan-2-yl)thiazol-2-yl)-4-(tetrahydro-2H-pyran-4-yl)pyridin-2-yl)amino)-3-(methyl-d₃)-2-oxo-2,3-dihydro-1H-imidazo[4,5-c]pyridin-1-yl)cyclopentyl-1-d)carbamate

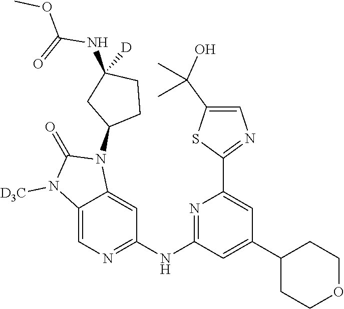 [SMILES: [2H]C([2H])([2H])n1c(=O)n([C@@H]2CC[C@]([2H])(NC(=O)OC)C2)c2cc(Nc3cc(C4CCOCC4)cc(-c4ncc(C(C)(C)O)s4)n3)ncc21]

According to Example 159, and Example 161. LCMS calculated for C₃₀H₃₄D₄N₇O₅S (M+H)⁺: m/z=612.3; found: 612.3.

### Example 164. Methyl ((1R,3R)-3-(6-((6′-(5-(2-hydroxypropan-2-yl)thiazol-2-yl)-4-(trifluoromethyl)-[3,4′-bipyridin]-2′-yl)amino)-3-(methyl-d₃)-2-oxo-2,3-dihydro-1H-imidazo[4,5-c]pyridin-1-yl)cyclopentyl-1-d)carbamate

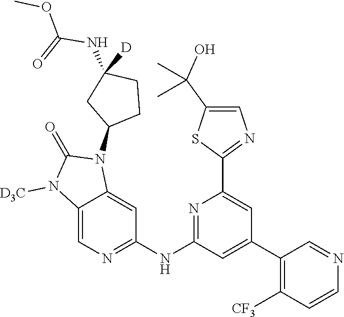 [SMILES: [2H]C([2H])([2H])n1c(=O)n([C@@H]2CC[C@@]([2H])(NC(=O)OC)C2)c2cc(Nc3cc(-c4cnccc4C(F)(F)F)cc(-c4ncc(C(C)(C)O)s4)n3)ncc21]

According to Example 92 (Step 4), Example 105 (Step 1-4), and Example 158 (Step 5-6), with ethyl 2-bromothiazole-5-carboxylate and 3-(4,4,5,5-tetramethyl-1,3,2-dioxaborolan-2-yl)-4-(trifluoromethyl)pyridine as starting materials. LCMS calculated for C₃₁H₂₈D₄F₃N₈O₄S (M+H)⁺: m/z=673.2; found: 673.3. ¹H NMR (600 MHz, DMSO) δ 10.65 (s, 1H), 9.00 (d, J=5.2 Hz, 1H), 8.84 (s, 1H), 8.25 (s, 1H), 7.96 (d, J=5.2 Hz, 1H), 7.81-7.78 (m, 2H), 7.67 (s, 1H), 7.39 (s, 1H), 7.24 (s, 1H), 5.02-4.93 (m, 1H), 3.53 (s, 3H), 2.35 (dd, J=13.6, 7.1 Hz, 1H), 2.23-2.06 (m, 3H), 1.94 (dd, J=13.6, 9.7 Hz, 1H), 1.63-1.58 (m, 1H), 1.60 (s, 6H).

### Example 165. Methyl ((1S,3R)-3-(6-((6′-(5-(2-hydroxypropan-2-yl)thiazol-2-yl)-4-(trifluoromethyl)-[3,4′-bipyridin]-2′-yl)amino)-3-(methyl-d₃)-2-oxo-2,3-dihydro-1H-imidazo[4,5-c]pyridin-1-yl)cyclopentyl-1-d)carbamate

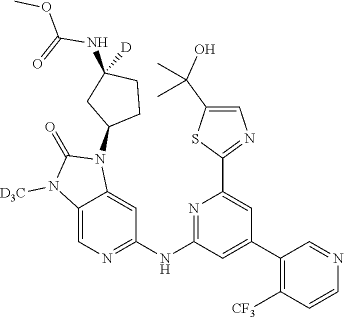 [SMILES: [2H]C([2H])([2H])n1c(=O)n([C@@H]2CC[C@]([2H])(NC(=O)OC)C2)c2cc(Nc3cc(-c4cnccc4C(F)(F)F)cc(-c4ncc(C(C)(C)O)s4)n3)ncc21]

According to Example 159, and Example 164. LCMS calculated for C₃₁H₂₈D₄F₃N₈O₄S (M+H)⁺: m/z=673.2; found: 673.2. ¹H NMR (600 MHz, DMSO) δ 10.59 (s, 1H), 9.00 (d, J=5.1 Hz, 1H), 8.83 (s, 1H), 8.26 (s, 1H), 8.07 (s, 1H), 7.96 (d, J=5.2 Hz, 1H), 7.79 (s, 1H), 7.65 (s, 1H), 7.40 (s, 1H), 7.27 (s, 1H), 4.85 (p, J=8.7 Hz, 1H), 3.53 (s, 3H), 2.36 (dd, J=13.0, 8.9 Hz, 1H), 2.29-2.19 (m, 1H), 2.17-2.11 (m, 1H), 2.07 (dd, J=13.0, 8.6 Hz, 1H), 1.95 (m, 1H), 1.84-1.77 (m, 1H), 1.59 (s, 6H).

### Example 166. Methyl ((1R,3R)-3-(6-((6-(5-(2-hydroxypropan-2-yl)thiazol-2-yl)-4-(1-methylpiperidin-4-yl)pyridin-2-yl)amino)-3-(methyl-d₃)-2-oxo-2,3-dihydro-1H-imidazo[4,5-c]pyridin-1-yl)cyclopentyl-1-d)carbamate

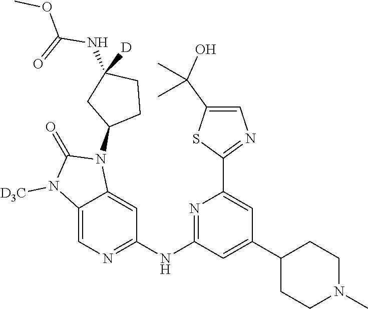

According to Example 92 (Step 4), Example 157, and Example 158 (Step 5-6), with ethyl 2-bromothiazole-5-carboxylate as starting materials. LCMS calculated for C₃₁H₃₇D₄N₈O₄S (M+H)⁺: m/z=625.3; found: 625.3.

### Example 167. Methyl ((1R,3R)-3-(6-((6′-(5-(2-hydroxypropan-2-yl)thiazol-2-yl)-2-(trifluoromethyl)-[3,4′-bipyridin]-2′-yl)amino)-3-(methyl-d₃)-2-oxo-2,3-dihydro-1H-imidazo[4,5-c]pyridin-1-yl)cyclopentyl-1-d)carbamate

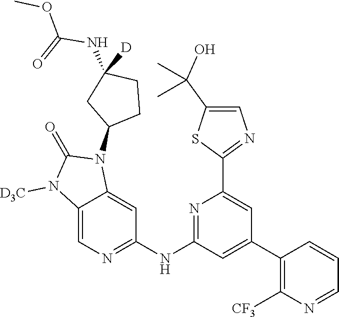 [SMILES: [2H]C([2H])([2H])n1c(=O)n([C@@H]2CC[C@@]([2H])(NC(=O)OC)C2)c2cc(Nc3cc(-c4cccnc4C(F)(F)F)cc(-c4ncc(C(C)(C)O)s4)n3)ncc21]

According to Example 92 (Step 4), Example 105 (Step 1-4), and Example 158 (Step 5), with ethyl 2-bromothiazole-5-carboxylate and 3-(4,4,5,5-tetramethyl-1,3,2-dioxaborolan-2-yl)-2-(trifluoromethyl)pyridine as starting materials. LCMS calculated for C₃₁H₂₈D₄F₃N₈O₄S (M+H)⁺: m/z=673.2; found: 673.2.

### Example 168. N-((1S,3R)-3-(6-((3-(1-(Difluoromethyl)-1H-pyrazol-4-yl)-2-methyl-8-(4-(trifluoromethyl)pyridin-3-yl)imidazo[1,2-b]pyridazin-6-yl)amino)-3-(methyl-d₃)-2-oxo-2,3-dihydro-1H-imidazo[4,5-c]pyridin-1-yl)-1-methylcyclopentyl)acetamide

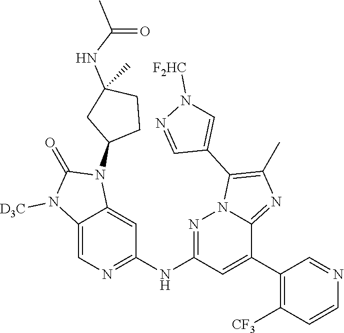 [SMILES: [2H]C([2H])([2H])n1c(=O)n([C@@H]2CC[C@](C)(NC(C)=O)C2)c2cc(Nc3cc(-c4cnccc4C(F)(F)F)c4nc(C)c(-c5cnn(C(F)F)c5)n4n3)ncc21]

According to Example 97 (Step 4), and Example 136. LCMS calculated for C₃₂H₂₈D₃F₅N₁₁O₂ (M+H)⁺: m/z=699.3; found: 699.3.

### Example 169. Methyl ((1R,3R)-3-(6-((3-(1-(difluoromethyl)-1H-pyrazol-4-yl)-7-(2-hydroxypropan-2-yl)-2-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate

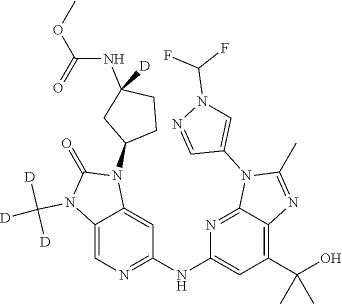 [SMILES: [2H]C([2H])([2H])n1c(=O)n([C@@H]2CC[C@@]([2H])(NC(=O)OC)C2)c2cc(Nc3cc(C(C)(C)O)c4nc(C)n(-c5cnn(C(F)F)c5)c4n3)ncc21]

According to Example 97 (Step 1), Example 115 (Step 5), and Example 158 (Step 5-6). LCMS calculated for C₂₈H₂₉D₄F₂N₁₀O₄ (M+H)⁺: m/z=615.3; found: 615.3.

### Example A. JAK2 LanthaScreen JH1 Binding Assay

JAK2 JH1 binding assay utilizes catalytic domain (JH1, amino acids 826-1132) of human JAK2 expressed as N-terminal FLAG-tagged, biotinylated protein in a baculovirus expression system (Carna Biosciences, Product #08-445-20N). The assay was conducted in black 384-well polystyrene plates in a final reaction volume of 20 μL. JAK2 JH1 (1.5 nM) was incubated with compounds (100 nL serially diluted in DMSO) in the presence of 50 nM fluorescent JAK2-JH1 tracer and 0.5 nM Streptavidin-Tb cryptate (Cisbio Part #610SATLB) in assay buffer (50 mM Tris, pH=7.5, 10 mM MgCl₂, 0.01% Brij-35, 0.1% BSA, 1 mM EGTA, 5% Glycerol and 5 mM DTT). Non-specific binding was accessed in the presence of 2 mM ATP. After incubation for 2 hours at 25° C., LanthaScreen signals were read on a PHERAstar FS plate reader (BMG LABTECH). Data was analyzed with IDBS XLfit and GraphPad Prism 5.0 software using a four parameter dose response curve to determine IC50 for each compound.

### Example B. JAK2 LanthaScreen JH2-WT Binding Assay

JAK2 JH2-WT binding assay utilizes pseudo-kinase domain (JH2, amino-acids 536-812 with 3 surface mutations W659A, W777A, F₇₉₄H) of human Wild Type JAK2 expressed as C-terminal His-Avi-tagged, biotinylated protein in a baculovirus expression system (BPS Bioscience, Catalog #79463). The assay was conducted in black 384-well polystyrene plates in a final reaction volume of 20 μL. JAK2 JH2-WT (0.145 nM) was incubated with compounds (100 nL serially diluted in DMSO) in the presence of 50 nM Fluorescent JAK2-JH2 Tracer (MedChem Express Catalog #HY-102055) and 0.25 nM Streptavidin-Tb cryptate (Cisbio Part #610SATLB) in assay buffer (50 mM Tris, pH=7.5, 10 mM MgCl₂, 0.01% Brij-35, 0.1% BSA, 1 mM EGTA, 5% Glycerol and 5 mM DTT). Non-specific binding was accessed in the presence of 2 mM ATP. After incubation for 1 hour at 25° C., LanthaScreen signals were read on a PHERAstar FS plate reader (BMG LABTECH). Data was analyzed with IDBS XLfit and GraphPad Prism 5.0 software using a four parameter dose response curve to determine IC50 for each compound.

### Example C. JAK2 LanthaScreen JH2-V617F Binding Assay

JAK2 JH2-V617F binding assay utilizes pseudo-kinase domain (JH2, amino-acids 536-812 with 3 surface mutations W659A, W777A, F794H) of human V617F mutant JAK2 expressed as C-terminal His-Avi-tagged, biotinylated protein in a baculovirus expression system (BPS Bioscience, Catalog #79498). The assay was conducted in black 384-well polystyrene plates in a final reaction volume of 20 μL. JAK2 JH2-V617F (0.26 nM) was incubated with compounds (100 nL serially diluted in DMSO) in the presence of 50 nM Fluorescent JAK2-JH2 Tracer (MedChem Express Catalog #HY-102055) and 0.25 nM Streptavidin-Tb cryptate (Cisbio Part #610SATLB) in assay buffer (50 mM Tris, pH=7.5, 10 mM MgCl₂, 0.01% Brij-35, 0.1% BSA, 1 mM EGTA, 5% Glycerol and 5 mM DTT). Non-specific binding was accessed in the presence of 2 mM ATP. After incubation for 1 hour at 25° C., LanthaScreen signals were read on a PHERAstar FS plate reader (BMG LABTECH). Data was analyzed with IDBS XLfit and GraphPad Prism 5.0 software using a four parameter dose response curve to determine IC50 for each compound.

### Example D. JAK2 HTRF Enzyme Activity Assay

JAK2 enzyme activity assays utilize catalytic domain (JH1, amino acids 808-1132) of human JAK2 expressed as N-terminal His-tagged protein in a baculovirus expression system (BPS Bioscience, Catalog #40450). The assays was conducted in black 384-well polystyrene plates in a final reaction volume of 20 μL. JAK2 (0.015 nM) was incubated with compounds (100 nL serially diluted in DMSO) in the presence of ATP (30 UM or 1 mM) and 500 nM Biotin-labeled EQEDEPEGDYFEWLE (SEQ ID NO.: 1) peptide (BioSource International, custom synthesis) in assay buffer (50 mM Tris, pH=7.5, 10 mM MgCl₂, 0.01% Brij-35, 0.1% BSA, 1 mM EGTA, 5% Glycerol and 5 mM DTT) for 60 minutes at 25° C. The reactions were stopped by the addition of 10 μL of detection buffer (50 mM Tris, pH 7.8, 0.5 mg/mL BSA, 150 mM NaCl), supplemented with EDTA, LANCE Eu-W1024 anti-phosphotyrosine (PY20), (PerkinElmer, Catalog #AD0067) and Streptavidin SureLight APC (PerkinElmer Catalog #CR130-100), for a final concentration of 15 mM, 1.5 nM and 75 nM, respectively. HTRF signals were read after 30 minutes incubation at room temperature on a PHERAstar FS plate reader (BMG LABTECH). Data was analyzed with IDBS XLfit and GraphPad Prism 5.0 software using a four parameter dose response curve to determine IC50 for each compound.

The compounds of the disclosure were tested in one or more of the assays described in Examples A-D, and the resulting data are shown in Table A.

TABLE A

| | | JH2 BIND | JH2 BIND | V617F ENZ |
|---|---|---|---|---|
| Example | JH1 BIND | WT | V617F | 30 UM |
| 1 | +++++ | ++ | +++ | +++++ |
| 2 | +++++ | + | + | +++++ |
| 3 | +++++ | + | + | ++++ |
| 4 | +++++ | + | + | +++++ |
| 5 | +++++ | + | + | +++++ |
| 6 | +++ | + | + | +++ |
| 7 | +++++ | + | ++ | +++++ |
| 8 | +++++ | + | + | +++++ |
| 9 | +++++ | + | + | +++++ |
| 10 | ++++ | + | + | +++ |
| 11 | +++++ | + | + | +++++ |
| 12 | +++++ | + | + | +++++ |
| 13 | ++++ | + | + | ++++ |
| 14 | +++++ | + | ++ | +++++ |
| 15 | +++++ | + | ++ | +++++ |
| 16 | +++++ | + | + | +++++ |
| 17 | +++++ | + | + | +++++ |
| 18 | +++++ | + | + | +++++ |
| 19 | +++++ | + | + | +++++ |
| 20 | +++++ | + | + | +++++ |
| 21 | +++++ | + | + | +++++ |
| 22 | +++++ | ++ | ++ | +++++ |
| 23 | +++++ | ++ | ++ | +++++ |
| 24 | +++++ | ++ | ++ | +++++ |
| 25 | +++++ | ++ | +++ | +++++ |
| 26 | +++++ | ++ | ++ | +++++ |
| 27 | +++++ | +++ | +++ | +++++ |
| 28 | +++++ | ++ | +++ | +++++ |
| 29 | +++++ | +++ | +++ | +++++ |
| 30 | +++++ | ++ | +++ | +++++ |
| 31 | +++++ | ++ | ++ | +++++ |
| 32 | +++++ | +++ | +++ | +++++ |
| 33 | +++++ | ++ | ++ | +++++ |
| 34 | +++++ | + | + | +++++ |
| 35 | +++++ | ++ | ++ | +++++ |
| 36 | +++++ | + | ++ | +++++ |
| 37 | +++++ | + | ++ | +++++ |
| 38 | +++++ | + | ++ | +++++ |
| 39 | +++++ | +++ | +++ | +++++ |
| 40 | +++++ | ++ | ++ | +++++ |
| 41 | +++++ | ++ | ++ | +++++ |
| 42 | +++++ | ++ | ++ | +++++ |
| 43 | +++++ | ++ | ++ | +++++ |
| 44 | +++++ | +++++ | +++++ | +++++ |
| 45 | +++++ | ++ | ++ | +++++ |
| 46 | +++++ | ++ | ++ | +++++ |
| 47 | +++++ | + | + | +++++ |
| 48 | +++++ | ++ | ++ | +++++ |
| 49 | +++++ | + | + | +++++ |
| 50 | +++++ | + | ++ | +++++ |
| 51 | +++++ | + | ++ | +++++ |
| 52 | ++++ | + | + | ++++ |
| 53 | +++++ | + | ++ | +++++ |
| 54 | +++++ | + | + | +++++ |
| 55 | +++++ | + | ++ | +++++ |
| 56 | +++++ | + | + | +++++ |
| 57 | +++++ | + | + | +++++ |
| 58 | +++++ | + | + | +++++ |
| 59 | +++++ | + | + | +++++ |
| 60 | +++++ | ++ | ++ | +++++ |
| 61 | +++++ | ++ | +++ | +++++ |
| 62 | +++++ | +++ | +++ | +++++ |
| 63 | +++++ | + | ++ | +++++ |
| 64 | +++++ | ++ | ++ | +++++ |
| 65 | +++++ | + | + | +++++ |
| 66 | +++++ | + | ++ | +++++ |
| 67 | +++++ | + | + | +++++ |
| 68 | +++++ | ++++ | ++++ | +++++ |
| 69 | +++++ | + | + | +++++ |
| 70 | +++++ | + | + | +++++ |
| 71 | +++++ | + | + | +++++ |
| 72 | +++++ | + | ++ | +++++ |
| 73 | +++++ | ++ | ++ | +++++ |
| 74 | +++++ | + | + | +++++ |
| 75 | +++++ | + | + | +++++ |
| 76 | +++++ | + | + | +++++ |
| 77 | +++++ | + | + | +++++ |
| 78 | +++++ | + | + | +++++ |
| 79 | +++++ | + | + | +++++ |
| 80 | +++++ | + | + | +++++ |
| 81 | +++++ | +++ | +++ | +++++ |
| 82 | +++++ | + | + | +++++ |
| 83 | +++++ | + | ++ | +++++ |
| 84 | +++++ | + | + | +++++ |
| 85 | +++++ | ++ | ++ | +++++ |
| 86 | +++++ | ++ | +++ | +++++ |
| 87 | +++++ | + | ++ | +++++ |
| 88 | +++++ | + | + | +++++ |
| 89 | +++++ | + | + | +++++ |
| 90 | +++ | + | + | +++++ |
| 91 | +++++ | + | + | ++ |
| 92 | +++++ | + | + | +++++ |
| 93 | +++++ | + | + | +++++ |
| 94 | +++++ | + | + | +++++ |
| 95 | +++++ | + | + | +++++ |
| 96 | +++++ | + | + | +++++ |
| 97 | +++++ | + | + | ++++ |
| 98 | +++++ | + | + | +++++ |
| 99 | +++++ | + | + | +++++ |
| 100 | +++++ | + | + | +++++ |
| 101 | +++++ | + | + | +++++ |
| 102 | +++++ | + | + | +++++ |
| 103 | +++++ | + | + | +++++ |
| 104 | +++++ | + | + | ++++ |
| 105 | +++++ | + | + | +++++ |
| 106 | +++++ | + | + | +++++ |
| 107 | +++++ | + | + | +++++ |
| 108 | +++++ | + | + | +++++ |
| 109 | | | + | +++++ |
| 110 | | | + | +++++ |
| 111 | | | + | +++++ |
| 112 | | | + | +++++ |
| 113 | | | + | +++++ |
| 114 | +++++ | + | + | +++++ |
| 115 | +++++ | + | + | +++++ |
| 116 | +++ | + | + | +++ |
| 117 | +++++ | + | + | +++++ |
| 118 | +++++ | + | + | +++++ |
| 119 | +++++ | + | + | +++++ |
| 120 | +++++ | + | + | +++++ |
| 121 | | | + | +++++ |
| 122 | | | + | +++++ |
| 123 | | | + | +++++ |
| 124 | | | + | +++++ |
| 125 | | | + | +++++ |
| 126 | | | + | +++++ |
| 127 | | | + | +++++ |
| 128 | | | + | +++++ |
| 129 | | | + | +++++ |
| 130 | | | + | +++++ |
| 131 | | | + | +++++ |
| 132 | | | + | +++++ |
| 133 | | | + | +++++ |
| 134 | | | + | +++++ |
| 135 | +++++ | + | + | ++++ |
| 136 | | | + | +++++ |
| 137 | | | + | +++++ |
| 138 | | | + | +++++ |
| 139 | | | + | +++++ |
| 140 | | | + | +++++ |
| 141 | | | + | +++++ |
| 142 | | | + | +++++ |
| 143 | | | + | +++++ |
| 144 | +++++ | + | + | +++++ |
| 145 | +++++ | + | + | +++++ |
| 146 | | | + | +++++ |
| 147 | | | + | +++++ |
| 148 | | | + | +++++ |
| 149 | | | + | +++++ |
| 150 | | | + | +++++ |
| 151 | | | + | +++++ |
| 152 | | | + | +++++ |
| 153 | | | + | +++++ |
| 154 | +++++ | + | + | +++++ |
| 155 | | | + | +++++ |
| 156 | +++++ | + | + | +++++ |
| 157 | +++++ | + | + | +++++ |
| 158 | | | + | +++++ |
| 159 | | | + | +++++ |
| 160 | | | + | +++++ |
| 161 | | | + | +++++ |
| 162 | | | + | +++++ |
| 163 | | | + | ++++ |
| 164 | | | + | +++++ |
| 165 | | | + | +++++ |
| 166 | | | + | +++++ |
| 167 | | | + | +++++ |
| 168 | | | + | +++++ |
| 169 | | | + | +++++ |

+ refers to IC₅₀ of ≤10 nM
++ refers to IC₅₀ of >10 nM to ≤100 nM
+++ refers to IC₅₀ of >100 nM to ≤500 nM
++++ refers to IC₅₀ of >500 nM to ≤1000 nM
+++++ refers to IC₅₀ of >1000 nM

### Example E. Cell Culture and STAT5 (Tyr694) Phosphorylation Cell Based Assay

Ba/F3 cells expressing human JAK2 V617F/EPOR (mouse JAK2 WT knocked out by CRISPR) are cultured in RPMI media with 10% FBS, 1 μg/mL Puromycin, 1 mg/mL Geneticin (Thermo Fisher). Ba/F3 cells expressing human JAK2 WT/EPOR are cultured in RPMI media with 10% FBS, 1 g/mL Puromycin, 1 mg/mL Geneticin and 2 ng/mL EPO. 24 hours before the assay, the culture medium for JAK2 V617F/EPOR Ba/F3 cells are changed to RPMI with 10% FBS without antibiotic (assay medium 1). Culture medium for Ba/F3 cells expressing human JAK2 WT/EPOR are changed to RPMI with 10% FBS and 2 ng/mL EPO (R&D systems) without antibiotic (assay medium 2). 50 nL/well test compounds in DMSO are transferred to the 384 white low volume cell culture plate (Greiner Bio-one) by ECHO liquid handler (Labcyte). The cells are centrifuged, resuspended in the corresponding fresh assay medium and dispensed at 10 μL/well (6×10⁶ cells/mL) with 0.5% DMSO in the final assay. After the treated cells are incubated at 37° C., 5% CO₂ for 2 hours, 4 μL/well supplemented lysis buffer (100× blocking buffer diluted 25 fold in 4× lysis buffer, Perkin-Elmer) are added and incubated at room temperature for 60 min with gentle shaking on orbital shaker at 600 rpm. Phospho-STAT5 Cryptate antibody and Phospho-STAT5 d2 antibody (1:1 vol/vol, Perkin-Elmer) are premixed and diluted 20 fold within the detection buffer. 4 μL of the premixed antibody solution are added to each well followed with 16 hours incubation at room temperature. The product activity is determined by measuring the fluorescence at 620 nm and 665 nm on Pherastar microplate reader (BMG Labtech). A ratio is calculated (665/620 nm) for each well. Wells with DMSO serve as the positive controls and wells containing high concentration of control compound are used as negative controls. IC₅₀ determination is performed by fitting the curve of percent control activity versus the log of the compound concentration using the Genedata Screener software.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A compound of Formula 1: 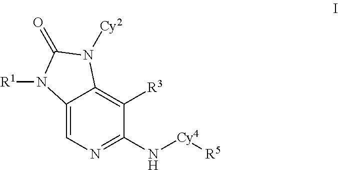
or a pharmaceutically acceptable salt thereof, wherein:
R¹ is selected from C₁₋₆ alkyl, C₂₋₆ alkenyl, and C₂₋₆ alkynyl, wherein the C₁₋₆ alkyl, C₂₋₆ alkenyl, and C₂₋₆ alkynyl of R¹ are each optionally substituted with 1, 2, or 3 independently selected R^{1A} substituents;
each R^{1A} is independently selected from halo, oxo, CN, NO₂, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{c11})R^{b11}, C(═NR^{c11})NR^{c11}R^{d11}, NR^{c11}C(═NR^{c11})NR^{c11}R^{d11}, NR^{c11}C(═NR^{c11})R^{b11}, NR^{c11}S(O)R^{b11}, NR^{c11}S(O)NR^{c11}R^{d11}, NR^{c11}S(O)₂R^{b11}, NR^{c11}S(O)(═NR^{c11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{c11})R^{b11}, and OS(O)₂R^{b11};
each R^{a11}, R^{b11}, R^{c11}, and R^{d11} is independently selected from H, C₁₋₆ alkyl, C₁₋₆ haloalkyl, C₂₋₆ alkenyl, and C₂₋₆ alkynyl, wherein the C₁₋₆ alkyl, C₂₋₆ alkenyl, and C₂₋₆ alkynyl of R^{a11}, R^{b11}, R^{c11} and R^{d11} are each optionally substituted with 1, 2, 3, 4, 5, 6, 7, or 8 independently selected R^{M} substituents;
each R^{c11} is independently selected from H, OH, CN, C₁₋₆ alkyl, C₁₋₆ alkoxy, C₁₋₆ haloalkyl, C₁₋₆ haloalkoxy, C₂₋₆ alkenyl, and C₂₋₆ alkynyl;
Cy² is selected from C₃₋₁₀ cycloalkyl and 4-12 membered heterocycloalkyl, wherein the C₃₋₁₀ cycloalkyl and 4-12 membered heterocycloalkyl are each substituted with 1, 2, 3, 4, 5, 6, 7 or 8 independently selected R² substituents;
each R² is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}C(═NR^{e2}) R^{b2}, NR^{c2}S(O)R^{b2}, NR^{c2}S(O)NR^{c2}R^{d2}, NR^{c2}S(O)₂R^{b2}, NR^{c2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, OS(O)₂R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, 4, 5, 6, 7, or 8 independently selected R^{2A} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a2}, R^{c2} and R^{d2} are each optionally substituted with 1, 2, 3, 4, 5, 6, 7, or 8 independently selected R^{2A} substituents;
or, any R^{c2} and R^{d2} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2A} substituents;
each R^{b2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b2} are each optionally substituted with 1, 2, 3, 4, 5, 6, 7, or 8 independently selected R^{2A} substituents;
each R^{e2} is independently selected from H, OH, CN, C₁₋₆ alkyl, C₁₋₆ alkoxy, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f2} and R^{g2} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h2} and Rⁱ² is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j2} and R^{k2} is independently selected from OH, C₁₋₆ alkoxy, and C₁₋₆ haloalkoxy;
or any R^{j2} and R^{k2} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a21}, SR^{a21}, NHOR^{a21}, C(O)R^{b21}, C(O)NR^{C21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}NR^{c21}R^{d21}, NR^{c21}C(O)R^{b21}, NR^{c21}C(O)OR^{a21}, NR^{c21}C(O)NR^{c21}R^{d21}, C(═NR^{e21})R^{b21}, C(═NR^{e21})NR^{c21}R^{d21}, NR^{c21}C(═NR^{e21})NR^{c21}R^{d21}, NR^{c21}C(═NR^{e21}) R^{b21}, NR^{c21}S(O)R^{b21}, NR^{c21}S(O)NR^{c21}R^{d21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, OS(O)₂R^{b21}, SF₅, P(O)R^{f21}R^{g21}, OP(O)(OR^{h21})(ORⁱ²¹), P(O)(OR^{h21})(ORⁱ²¹), and BR^{j21}R^{k21}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{2A} are each optionally substituted with 1, 2, 3, 4, 5, 6, 7, or 8 independently selected R^{2B} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a21}, R^{e21} and R^{d21} are each optionally substituted with 1, 2, 3, 4, 5, 6, 7, or 8 independently selected R^{2B} substituents;
or, any R^{c21} and R^{d21} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2B} substituents;
each R^{b21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b21} are each optionally substituted with 1, 2, 3, 4, 5, 6, 7, or 8 independently selected R^{2B} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f21} and R^{g21} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h21} and Rⁱ²¹ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j21} and R^{k21} is independently selected from OH, C₁₋₆ alkoxy, and C₁₋₆ haloalkoxy;
or any Rⁱ²¹ and R^{k21} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2B} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a22}, SR^{a22}, NHOR^{a22}, C(O)R^{b22}, C(O)NR^{c22}R^{d22}, C(O)NR^{c22}(OR^{a22}), C(O)OR^{a22}, OC(O)R^{b22}, OC(O)NR^{c22}R^{d22}, NR^{c22}R^{d22}, NR^{c22}NR^{c22}R^{d22}, NR^{c22}C(O)R^{b22}, NR^{c22}C(O)OR^{a22}, NR^{c22}C(O)NR^{c22}R^{d22}, C(═NR^{e22})R^{b22}, C(═NR^{e22})NR^{c22}R^{d22}, NR^{c22}C(═NR^{e22})NR^{c22}R^{d22}, NR^{c22}C(═NR^{e22}R^{d22}, NR^{c22}S(O)R^{b22}, NR^{c22}S(O)NR^{c22}R^{d22}, NR^{c22}S(O)₂R^{b22}, NR^{c22}S(O)(═NR^{e22})R^{b22}, NR^{c22}S(O)₂NR^{c22}R^{d22}, S(O)R^{b22}, S(O)NR^{c22}R^{d22}, S(O)₂R^{b22}, S(O)₂NR^{c22}R^{d22}, OS(O)(═NR^{e22}) R^{b22}, and OS(O)₂R^{b22}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl- of R^{2C} are each optionally substituted with 1, 2, 3, or 4 independently selected R^{M} substituents;
each R^{a22}, R^{c22}, and R^{d22} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a22}, R^{c22} and R^{d22} are each optionally substituted with 1, 2, 3, or 4 independently selected R^{M} substituents;
or, any R^{e22} and R^{d22} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b22} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b22} are each optionally substituted with 1, 2, 3, or 4 independently selected R^{M} substituents;
each R^{e22} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
R³ is selected from H, halo, C₁₋₆ alkyl, C₂₋₆ alkenyl, and C₂₋₆ alkynyl;
Cy⁴ is selected from C₆₋₁₀ aryl and 5-14 membered heteroaryl, wherein the C₆₋₁₀ aryl and 5-12 membered heteroaryl are each optionally substituted with 1, 2, 3, 4, 5, 6, 7 or 8 independently selected R⁴ substituents;
each R⁴ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a4}, SR^{a4}, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NR^{e4})NR^{c4}R^{d4}, NR^{c4}C(═NR^{e4})NR^{c4}R^{d4}, NR^{c4}C(═NR^{e4})R^{b4}, NR^{c4}S(O)R^{b4}, NR^{c4}S(O)NR^{c4}R^{d4}, NR^{c4}S(O)₂R^{b4}, NR^{c4}S(O)(═NR^{e4})R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, OS(O)(═NR^{e4})R^{b4}, OS(O)₂R^{b4}, SF₅, P(O)R^{f4}R^{g4}, OP(O)(OR^{h4})(ORⁱ⁴), P(O)(OR^{h4})(ORⁱ⁴), and BR^{j4}R^{k4}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R⁴ are each optionally substituted with 1, 2, 3, 4, 5, 6, 7, or 8 independently selected R^{4A} substituents;
each R^{a4}, R^{c4}, and R^{d4} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a4}, R^{c4} and R^{d4} are each optionally substituted with 1, 2, 3, 4, 5, 6, 7, or 8 independently selected R^{4A} substituents;
or, any R^{c4} and R^{d4} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{4A} substituents;
each R^{b4} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b4} are each optionally substituted with 1, 2, 3, 4, 5, 6, 7, or 8 independently selected R^{4A} substituents;
each R^{e4} is independently selected from H, OH, CN, C₁₋₆ alkyl, C₁₋₆ alkoxy, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f4} and R^{g4} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h4} and Rⁱ⁴ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j4} and R^{k4} is independently selected from OH, C₁₋₆ alkoxy, and C₁₋₆ haloalkoxy;
or any R^{j4} and R^{k4} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{4A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a41}, SR^{a41}, NHOR^{a41}, C(O)R^{b41}, C(O)NR^{c41}R^{d41}, C(O)NR^{c41}(OR^{a41}), C(O)OR^{a41}, OC(O)R^{b41}, OC(O)NR^{c41}R^{d41}, NR^{c41}R^{d41}, NR^{c41}NR^{c41}R^{d41}, NR^{c41}C(O)R^{b41}, NR^{c41}C(O)OR^{a41}, NR^{c41}C(O)NR^{c41}R^{d41}, C(═NR^{e41})R^{b41}, C(═NR^{e41})NR^{c41}R^{d41}, NR^{c41}C(═NR^{e41})NR^{c41}R^{d41}, NR^{c41}C(═NR^{e41})R^{b41}, NR^{c41}S(O)R^{b41}, NR^{c41}S(O)NR^{c41}R^{d41}, NR^{c41}S(O)₂R^{b41}, NR^{c41}S(O)(═NR^{e41})R^{b41}, NR^{c41}S(O)₂NR^{c41}R^{d41}, S(O)R^{b41}, S(O)NR^{c41}R^{d41}, S(O)₂R^{b41}, S(O)₂NR^{c41}R^{d41}, OS(O)(═NR^{e41})R^{b41}, OS(O)₂R^{b41}, SF₅, P(O)R^{f41}R^{g41}, OP(O)(OR^{h41})(ORⁱ⁴¹), P(O)(OR^{h41})(ORⁱ⁴¹), and BR^{j41}R^{k41}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R⁴¹ are each optionally substituted with 1, 2, 3, 4, 5, 6, 7, or 8 independently selected R^{4B} substituents;
each R^{a41}, R^{c41}, and R^{d41} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a41}, R^{c41} and R^{d41} are each optionally substituted with 1, 2, 3, 4, 5, 6, 7, or 8 independently selected R^{4B} substituents;
or, any R^{c41} and R^{d41} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{4B} substituents;
each R^{b41} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b41} are each optionally substituted with 1, 2, 3, 4, 5, 6, 7, or 8 independently selected R^{4B} substituents;
each R^{c41} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f41} and R^{g41} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h41} and Rⁱ⁴¹ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j41} and R^{k41} is independently selected from OH, C₁₋₆ alkoxy, and C₁₋₆ haloalkoxy;
or any R^{j41} and R^{k41} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{4B} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a42}, SR^{a42}, NHOR^{a42}, C(O)R^{b42}, C(O)NR^{c42}R^{d42}, C(O)NR^{c42}(OR^{a42}), C(O)OR^{a42}, OC(O)R^{b42}, OC(O)NR^{c42}R^{d42}, NR^{c42}R^{d42}, NR^{c42}NR^{c42}R^{d42}, NR^{c42}C(O)R^{b42}, NR^{c42}C(O)OR^{a42}, NR^{c42}C(O)NR^{c42}R^{d42}, C(═NR^{e42})R^{b42}, C(═NR^{e42})NR^{c42}R^{d42}, NR^{c42}C(═NR^{e42})NR^{c42}R^{d42}, NR^{c42}C(═NR^{e42})R^{b42}, NR^{c42}S(O)R^{b42}, NR^{c42}S(O)NR^{c42}R^{d42}, NR^{c42}S(O)₂R^{b42}, NR^{c42}S(O)(═NR^{e42}) R^{b42}, NR^{c42}S(O)₂NR^{c42}R^{d42}, S(O)R^{b42}, S(O)NR^{c42}R^{d42}, S(O)₂R^{b42}, S(O)₂NR^{c42}R^{d42}, OS(O)(═NR^{e42})R^{b42}, and OS(O)₂R^{b42}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{4C} are each optionally substituted with 1, 2, 3, or 4 independently selected R^{M} substituents;
each R^{a42}, R^{c42}, and R^{d42} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a42}, R^{c42} and R^{d42} are each optionally substituted with 1, 2, 3, or 4 independently selected R^{M} substituents;
or, any R^{c42} and R^{d42} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b42} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b42} are each optionally substituted with 1, 2, 3, or 4 independently selected R^{M} substituents;
each R^{e42} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
R⁵ is selected from C₁₋₆ alkyl, C₂₋₆ alkenyl, C₂₋₆ alkynyl, C₃₋₁₂ cycloalkyl, C₆₋₁₀ aryl, 5-12 membered heteroaryl, 4-12 membered heterocycloalkyl, OR^{w2}, SR^{w2}, NR^{w2}R^{w3}, C(O)R^{w2}, C(O)NR^{w2}R^{w3}, C(O)OR^{w2}, OC(O)R^{w2}, OC(NR^{w3})R^{w2}, OC(O)NR^{w2}R^{w3}, NR^{w3}C(O)R^{w2}, NR^{w3}C(O)OR^{w2}, NR^{w3}C(O)NR^{w2}R^{w3}, NR^{w3}S(O)R^{w2}, NR^{w3}S(O)NR^{w2}R^{w3}, NR 3S(O)₂R^{w2}, S(O)R^{w2}, S(O)NR^{w2}R^{w3}, S(O)₂R^{w2}, and S(O)₂NR^{w2}R^{w3};
wherein the C₁₋₆ alkyl and C₆₋₁₀ aryl of R⁵ are each substituted with 1 R^{w1} substituent and optionally substituted 1, 2, 3, 4, 5, 6, 7, or 8 independently selected R^{5A} substituents; or
the C₆₋₁₀ aryl of R⁵ is substituted with 1 R^{w5} substituent and optionally substituted 1, 2, 3, 4, 5, 6, 7, or 8 independently selected R^{5A} substituents; and
the C₂₋₆ alkenyl, C₂₋₆ alkynyl, C₃₋₁₂ cycloalkyl, 5-12 membered heteroaryl, and 4-12 membered heterocycloalkyl, of R⁵ are each optionally substituted with 1, 2, 3, 4, 5, 6, 7, or 8 independently selected R^{5A} substituents;
R^{w1} is selected from C₂₋₆ alkenyl, C₂₋₆ alkynyl, C₃₋₁₂ cycloalkyl, 5-12 membered heteroaryl, C₆₋₁₂ aryl, 4-12 membered heterocycloalkyl, OR^{w4}, NHR^{w4}, NR^{w3}R^{w4}, SR^{w4}, S(O)₂R^{w4}, C(O)R^{w4}, C(O)NHR^{w4}, C(O)NR^{w3}R^{w4}, OC(O)R^{w4}, S(O)R^{w3}, S(O)NR^{w3}R^{w3}, S(O)₂R^{w2}, and S(O)—NR^{w3}R³, wherein the C₂₋₆ alkenyl, C₂₋₆ alkynyl, C₃₋₁₂ cycloalkyl, 5-12 membered heteroaryl, C₆₋₁₂ aryl, and 4-12 membered heterocycloalkyl of R^{w1} are each optionally substituted with 1, 2, 3, 4, 5, 6, 7, or 8 independently selected R^{5A} substituents;
each R^{w2} is independently selected from C₃₋₁₂ cycloalkyl, 5-12 membered heteroaryl, and 4-12 membered heterocycloalkyl, wherein the C₃₋₁₂ cycloalkyl, 5-12 membered heteroaryl, and 4-12 membered heterocycloalkyl of R^{w2} are each optionally substituted with 1, 2, 3, 4, 5, 6, 7, or 8 independently selected R^{5A} substituents;
each R^{w3} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{w3} are each optionally substituted with 1, 2, 3, 4, 5, 6, 7, or 8 independently selected R^{5A} substituents;
each R^{w4} is independently selected from C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a51}, R^{c51} and R^{d51} are each optionally substituted with 1, 2, 3, 4, 5, 6, 7, or 8 independently selected R^{5B} substituents;
R^{w5} is C₁₋₆ alkyl which is substituted with oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{c51}R^{d51}, NR^{c51}NR^{c51}R^{d51}, NR^{c51}C(O)R^{b51}, NR^{c51}C(O)OR^{a51}, NR^{c51}C(O)NR^{c51}R^{d51}, C(═NR^{e51})R^{b51}, C(═NR^{e51})NR^{c51}R^{d51}, NR^{c51}C(═NR^{e51})NR^{c51}R^{d51}, NR^{c51}C(═NR^{e51})R^{b51}, NR^{c51}S(O)R^{b51}, NR^{c51}S(O)NR^{c51}R^{d51}, NR^{c51}S(O)₂R^{b51}, NR^{c51}S(O)(═NR^{e51})R^{b51}, NR^{c51}S(O)₂NR^{c51}R^{d51}, S(O)R^{b51}, S(O)NR^{c51}R^{d51}, S(O)₂R^{b51}, S(O)₂NR^{c51}R^{d51}, OS(O)(═NR^{e51})R^{b51}, OS(O)₂R^{b51}, SF₅, P(O)R^{f51}R^{g51}, OP(O)(OR^{h51})(ORⁱ⁵¹), P(O)(OR^{h51})(ORⁱ⁵¹), or BR^{j51}R^{k51}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{w5} are each optionally substituted with 1, 2, 3, 4, 5, 6, 7, or 8 independently selected R^{5B} substituents;
each R^{5A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{c51}R^{d51}, NR^{c51}NR^{c51}R^{d51}, NR^{c51}C(O)R^{b51}, NR^{c51}C(O)OR^{a51}, NR^{c51}C(O)NR^{c51}R^{d51}, C(═NR^{e51})R^{b51}, C(═NR^{e51})NR^{c51}R^{d51}, NR^{c51}C(═NR^{e51})NR^{c51}R^{d51} NR^{c51}C(═NR^{e51})R^{b51}, NR^{c51}S(O)R^{d51}, NR^{c51}S(O)NR^{c51}R^{d51}, NR^{c51}S(O)₂R^{b51}, NR^{c51}S(O)(═NR^{e51})R^{b51}, NR^{c51}S(O)₂NR^{c51}R^{d51}, S(O)R^{b51}, S(O)NR^{c51}R^{d51}, S(O)₂R^{b51}, S(O)₂NR^{c51}R^{d51}, OS(O)(═NR^{e51})R^{b51}, OS(O)₂R^{b51}, SF₅, P(O)R^{f51}R^{g51}, OP(O)(OR^{h51})(ORⁱ⁵¹), P(O)(OR^{h51})(ORⁱ⁵¹), and BRⁱ⁵¹R^{k51}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{5A} are each optionally substituted with 1, 2, 3, 4, 5, 6, 7, or 8 independently selected R^{5B} substituents;
each R^{a51}, R^{c51}, and R^{d51} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a51}, R^{e51} and R^{d51} are each optionally substituted with 1, 2, 3, 4, 5, 6, 7, or 8 independently selected R^{5B} substituents;
or, any R^{c51} and R^{d51} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{5B} substituents;
each R^{b51} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b51} are each optionally substituted with 1, 2, 3, 4, 5, 6, 7, or 8 independently selected R^{5B} substituents;
each R^{e51} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f51} and R^{g51} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h51} and Rⁱ⁵¹ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j51} and R^{k51} is independently selected from OH, C₁₋₆ alkoxy, and C₁₋₆ haloalkoxy;
or any R^{j51} and R^{k51} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{5B} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a52}, SR^{a52}, NHOR^{a52}, C(O)R^{b52}, C(O)NR^{c52}R^{d52}, C(O)NR^{c52}(OR^{a52}), C(O)OR^{a52}, OC(O)R^{b52}, OC(O)NR^{c52}R^{d52}, NR^{c52}R^{d52}, NR^{c52}NR^{c52}R^{d52}, NR^{c52}C(O)R^{b52}, NR^{c52}(O)OR^{a52}, NR^{c52}C(O)NR^{c52}R^{d52}, C(═NR^{e52}) R^{b52}, C(═NR^{e52})NR^{d52}R^{d52}, NR^{c52}C(═NR^{e52}) NR^{c52}R^{d52}, NR^{c52}C(═NR^{e52}) R^{b52}, NR^{c52}S(O)R^{b52}, NR^{c52}S(O)NR⁵²R^{d52}, NR⁵²S(O)₂R^{b52}, NR^{c52}S(O)(═NR^{e52}) R^{b52}, NR^{c52}S(O)₂NR^{c52}R^{d52}, S(O)R^{b52}, S(O)NR^{c52}R^{d52}, S(O)₂R^{b52}, S(O)₂NR^{c52}R^{d52}, OS(O)(═NR^{e52})R^{b52}, and OS(O)₂R^{b52}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{5B} are each optionally substituted with 1, 2, 3, or 4 independently selected R^{M} substituents;
each R^{a52}, R^{c52}, and R^{d52} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a52}, R^{e52} and R^{d52} are each optionally substituted with 1, 2, 3, or 4 independently selected R^{M} substituents;
or, any R^{c52} and R^{d52} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b52} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b52} are each optionally substituted with 1, 2, 3, or 4 independently selected R^{M} substituents;
each R^{c52} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, and
each R^{M} is independently selected from H, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
provided that:
(i) Cy⁴ is not triazolopyridinyl; and
(ii) R⁵ is not morpholinyl or piperazinyl; and
(iii) when Cy⁴ is phenyl, R⁵ is not pyridinyl or pyrimidinyl.

2. The compound of claim 1, or a pharmaceutically acceptable salt thereof, wherein the compound of Formula I is a compound of Formula II: 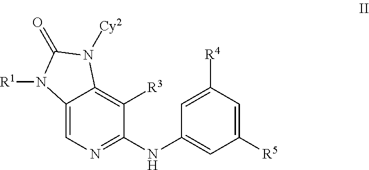
or a pharmaceutically acceptable salt thereof, wherein:
R⁴ is selected from C₆₋₁₀ aryl, C₃₋₁₀ cycloalkyl, 5-10 membered heteroaryl, 4-10 membered heterocycloalkyl, OR^{a4}, and NR^{c4}C(O)OR^{a4}, wherein the C₆₋₁₀ aryl, C₃₋₁₀ cycloalkyl, 5-10 membered heteroaryl, and 4-10 membered heterocycloalkyl of R⁴ are each optionally substituted with 1, 2, 3, or 4 independently selected R^{4A} substituents;
each R^{a4}, R^{c4}, and R^{d4} is independently selected from H, C₁₋₆ alkyl, C₁₋₆ haloalkyl, C₂₋₆ alkenyl, and C₂₋₆ alkynyl;
each R^{4A} is independently selected from C₁₋₆ alkyl, C₁₋₆ haloalkyl, C₂₋₆ alkenyl, C₂₋₆ alkynyl, and CN;
R⁵ is selected from triazolyl, pyrazolyl, piperidinyl, tetrahydropyranyl, NR^{w2}R^{w3}, NR^{w3}C(O)R^{w2}, NR^{w3}C(O)OR^{w2}, and NR^{w3}S(O)₂R^{w2}, wherein the triazolyl, pyrazolyl, piperidinyl, and tetrahydropyranyl of R⁵ are each optionally substituted with 1, 2, 3, or 4 independently selected R^{5A} substituents;
each R^{w2} is independently selected from C₃₋₁₂ cycloalkyl, 5-12 membered heteroaryl, and 4-12 membered heterocycloalkyl, wherein the C₃₋₁₂ cycloalkyl, 5-12 membered heteroaryl, and 4-12 membered heterocycloalkyl of R^{w2} are each optionally substituted with 1, 2, 3, or 4 independently selected R^{5A} substituents;
each R^{w3} is independently selected from H, C₁₋₆ alkyl, C₁₋₆ haloalkyl, C₂₋₆ alkenyl, and C₂₋₆ alkynyl;
each R^{5A} is independently selected from halo, oxo, C₁₋₆ alkyl, C₁₋₆ haloalkyl, C₂₋₆ alkenyl, C₂₋₆ alkynyl, CN, NO₂, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{c51}R^{d51}, NR^{c51}NR^{c51}R^{d51}, NR^{c51}C(O)R^{b51}, NR^{c51}C(O)OR^{a51}, NR^{c51}C(O)NR^{c51}R^{d51}, NR^{c51}S(O)R^{b51}, NR^{c51}S(O)NR^{c51}R^{d51}, NR^{c51}S(O)₂R^{b51}, NR^{c51}S(O)₂NR^{c51}R^{d51}, S(O)R^{b51}, S(O)NR^{c51}R^{d51}, S(O)₂R^{b51}, and S(O)₂NR^{c51}R^{d51}; and
each R^{a51}, R^{b51}, R^{c51}, and R^{d51} is independently selected from H, C₁₋₆ alkyl, C₁₋₆ haloalkyl, C₂₋₆ alkenyl, and C₁₋₆ alkynyl.

3. The compound of claim 1, or a pharmaceutically acceptable salt thereof, wherein R⁴ is selected from phenyl, C₃₋₇ cycloalkyl, 5-6 membered heteroaryl, 4-7 membered heterocycloalkyl, OR^{a4}, and NR^{c4}C(O)OR^{a4}, wherein the phenyl, C₃₋₇ cycloalkyl, 5-6 membered heteroaryl, and 4-7 membered heterocycloalkyl of R⁴ are each optionally substituted with 1, 2, 3, or 4 independently selected R^{4A} substituents.

4. The compound of claim 1, or a pharmaceutically acceptable salt thereof, wherein R⁴ is selected from phenyl, 5-6 membered heteroaryl, 4-7 membered heterocycloalkyl, OR^{a4}, and NR^{c4}C(O)OR^{a4}, wherein the phenyl, 5-6 membered heteroaryl, and 4-7 membered heterocycloalkyl of R⁴ are each optionally substituted with 1, 2, 3, or 4 independently selected R^{4A} substituents.

5. The compound of claim 1, or a pharmaceutically acceptable salt thereof, wherein R⁴ is selected from phenyl, tetrahydropyranyl, pyrazolyl, OR^{a4}, and NR^{c4}C(O)OR^{a4}, wherein the phenyl, tetrahydropyranyl, and pyrazolyl of R⁴ are each optionally substituted with 1, 2, 3, or 4 independently selected R^{4A} substituents; and
each R^{a4} and R^{c4} is independently selected from H, C₁₋₆ alkyl, C₁₋₆ haloalkyl, C₂₋₆ alkenyl, and C₂₋₆ alkynyl.

6. The compound of claim 1, or a pharmaceutically acceptable salt thereof, wherein each R^{4A} is independently selected from C₁₋₆ alkyl, C₁₋₆ haloalkyl, and CN.

7. The compound of claim 1, or a pharmaceutically acceptable salt thereof, wherein each R^{4A} is independently selected from C₁₋₆ alkyl and CN.

8. The compound of claim 1, or a pharmaceutically acceptable salt thereof, wherein each R^{4A} is independently selected from methyl and CN.

9. The compound of claim 1, or a pharmaceutically acceptable salt thereof, wherein R⁴ is selected from phenyl, tetrahydropyranyl, pyrazolyl, OR^{a4}, and NR^{c4}C(O)OR^{a4}, wherein the phenyl, tetrahydropyranyl, and pyrazolyl of R⁴ are each optionally substituted with 1 or 2 independently selected R^{4A} substituents; and
each R^{a4} and R^{c4} is independently selected from H and C₁₋₆ alkyl; and
each R^{4A} is independently selected from C₁₋₆ alkyl and CN.

10. The compound of claim 1, or a pharmaceutically acceptable salt thereof, wherein R⁵ is selected from triazolyl, pyrazolyl, piperidinyl, tetrahydropyranyl, NHR^{w2}, NHC(O)R^{w2}, NHC(O)OR^{w2}, and NHS(O)₂R^{w2}, wherein the triazolyl, pyrazolyl, piperidinyl, and tetrahydropyranyl of R⁵ are each optionally substituted with 1, 2, 3, or 4 independently selected R^{5A} substituents.

11. The compound of claim 1, or a pharmaceutically acceptable salt thereof, wherein each R^{w2} is independently selected from C₃₋₇ cycloalkyl, 5-6 membered heteroaryl, and 4-7 membered heterocycloalkyl, wherein the C₃₋₇ cycloalkyl, 5-6 membered heteroaryl, and 4-7 membered heterocycloalkyl of R^{w2} are each optionally substituted with 1, 2, 3, or 4 independently selected R^{5A} substituents.

12. The compound of claim 1, or a pharmaceutically acceptable salt thereof, wherein each R^{w2} is independently selected from cyclopropyl, cyclobutyl, oxazolyl, and tetrahydropyranyl, wherein the cyclopropyl, cyclobutyl, oxazolyl, and tetrahydropyranyl of R^{w2} are each optionally substituted with 1, 2, 3, or 4 independently selected R^{5A} substituents.

13. The compound of claim 1, or a pharmaceutically acceptable salt thereof, wherein each R^{5A} is independently selected from halo, C₁₋₆ alkyl, C₁₋₆ haloalkyl, CN, OR^{a51}, C(O)R^{b51}, and C(O)OR^{a51}; and
each R^{a51} and R^{b51} is independently selected from H, C₁₋₆ alkyl, C₁₋₆ haloalkyl, C₂₋₆ alkenyl, and C₂₋₆ alkynyl.

14. The compound of claim 1, or a pharmaceutically acceptable salt thereof, wherein each R^{5A} is independently selected from halo, C₁₋₆ alkyl, and C(O)OR^{a51}; and
each R^{a51} is independently selected from H and C₁₋₆ alkyl.

15. The compound of claim 1, or a pharmaceutically acceptable salt thereof, wherein each R^{5A} is independently selected from fluoromethyl, and ethoxycarbonyl.

16. The compound of claim 1, or a pharmaceutically acceptable salt thereof, wherein R⁵ is selected from triazolyl, pyrazolyl, piperidinyl, tetrahydropyranyl, NHR^{w2}, NHC(O)R^{w2}, NHC(O)OR^{w2}, and NHS(O)₂R^{w2}, wherein the triazolyl, pyrazolyl, piperidinyl, and tetrahydropyranyl of R⁵ are each optionally substituted with 1, 2, 3, or 4 independently selected R^{5A} substituents;
each R^{w2} is independently selected from cyclopropyl, cyclobutyl, oxazolyl, and tetrahydropyranyl, wherein the cyclopropyl, cyclobutyl, oxazolyl, and tetrahydropyranyl of R^{w2} are each optionally substituted with 1, 2, 3, or 4 independently selected R^{5A} substituents;
each R^{5A} is independently selected from halo, C₁₋₆ alkyl, and C(O)OR^{a51}; and
each R^{a51} is independently selected from H and C₁₋₆ alkyl.

17. The compound of claim 1, or a pharmaceutically acceptable salt thereof, wherein Cy⁴ is selected from thiazolyl, pyrazolyl, pyridinyl, and pyrimidinyl, wherein the thiazolyl, pyrazolyl, pyridinyl, and pyrimidinyl of Cy⁴ are each optionally substituted with 1, 2, 3, or 4 independently selected R⁴ substituents.

18. The compound of claim 1, wherein the compound of Formula I is a compound of Formula IIIa, IIIb, IIIc, IIId, IIIe, IIIf, IIIg, IIIh, IIIi, IIIj, IIIk, IIIm, or IIIn: 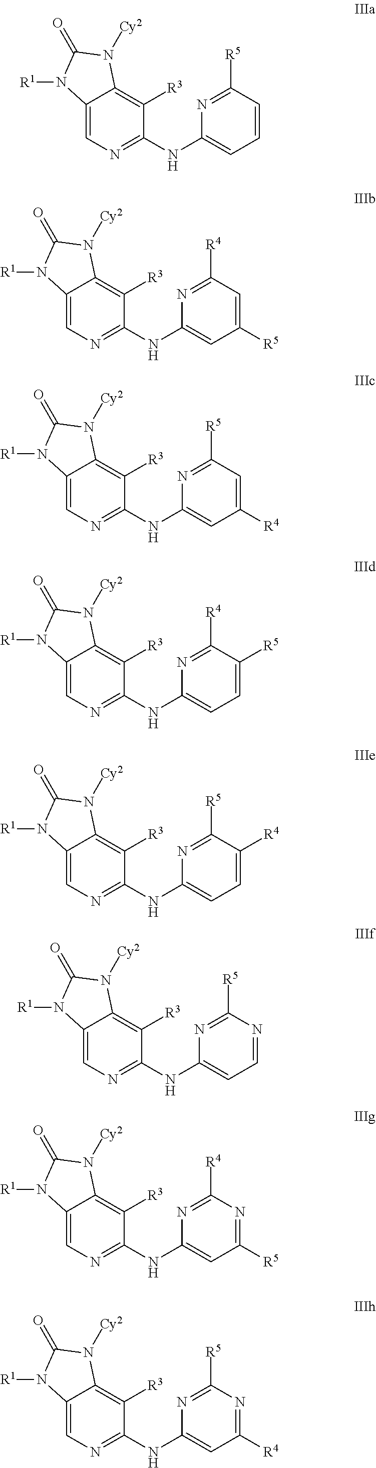 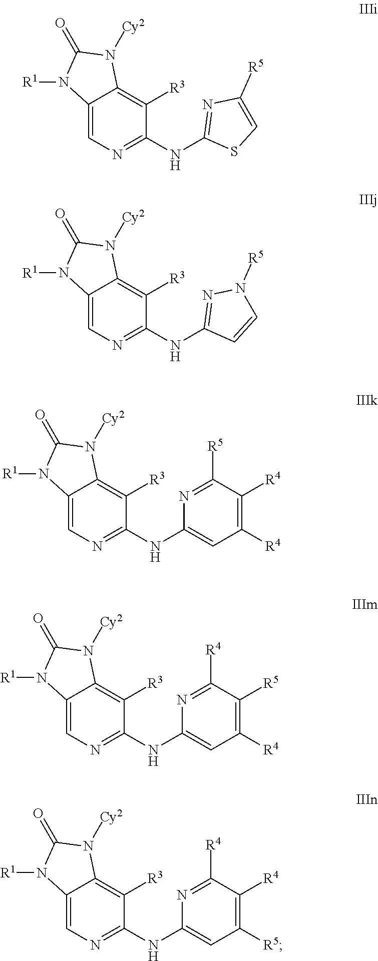
or a pharmaceutically acceptable salt thereof.

19. The compound of claim 1, or a pharmaceutically acceptable salt thereof, wherein:
each R⁴ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, (4-10 membered heterocycloalkyl)-C₁₋₆ alkyl-, OR^{a4}, and C(O)R^{a4}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, and (4-10 membered heterocycloalkyl)-C₁₋₆ alkyl- of R⁴ are each optionally substituted with 1, 2, 3, or 4 independently selected R^{4A} substituents;
each R^{a4} and R^{b4} is independently selected from H, C₁₋₆ alkyl, C₁₋₆ haloalkyl, C₂₋₆ alkenyl, C₂₋₆ alkynyl, and 4-6 membered heterocycloalkyl:
each R^{4A} is independently selected from halo, oxo, C₁₋₆ alkyl, C₁₋₆ haloalkyl, C₂₋₆ alkenyl, C₂₋₆ alkynyl, CN, OR^{a41}, and C(O)R^{b41};
each R^{a41} and R^{b41} is independently selected from H, C₁₋₆ alkyl, and C₁₋₆ haloalkyl;
R⁵ is selected from C₁₋₆ alkyl, C₃₋₁₂ cycloalkyl, C₆₋₁₀ aryl, 5-12 membered heteroaryl, tetrahydropyranyl, pyrrolidinyl, piperidinyl, 2,3-dihydrobenzo[b][1,4]dioxinyl, 3,4-dihydro-2H-benzo[b][1,4]oxazinyl, azaspiro[2,4]heptanyl, OR^{w2}, NR^{w2}R^{w3}, C(O)R^{w2}, and C(O)NR^{w2}R^{w3};
wherein the C₁₋₆ alkyl and C₆₋₁₀ aryl of R⁵ are each substituted with 1 R^{w1} substituent and optionally substituted 1 or 2 independently selected R^{5A} substituents; or
the C₆₋₁₀ aryl of R⁵ is substituted with 1 R^{w5} substituent and optionally substituted 1 or 2 independently selected R^{5A} substituents; and
the C₃₋₁₂ cycloalkyl, 5-12 membered heteroaryl, tetrahydropyranyl, pyrrolidinyl, piperidinyl, 2,3-dihydrobenzo[b][1,4]dioxinyl, 3,4-dihydro-2H-benzo[b][1,4]oxazinyl, and azaspiro[2.4]heptanyl of R⁵ are each optionally substituted with 1, 2, 3, or 4 independently selected R^{5A} substituents;
R^{w1} is selected from C₃₋₁₂ cycloalkyl, 4-12 membered heterocycloalkyl, OR^{w4}, and NHR^{w4}, wherein the C₃₋₁₂ cycloalkyl and 4-12 membered heterocycloalkyl or R^{w1} are each optionally substituted by 1, 2, 3, or 4 independently selected R^{5A} substituents;
each R^{w2} is independently selected from C₃₋₁₂ cycloalkyl, 5-12 membered heteroaryl, and 4-12 membered heterocycloalkyl, wherein the C₃₋₁₂ cycloalkyl, 5-12 membered heteroaryl, and 4-12 membered heterocycloalkyl of R^{w2} are each optionally substituted with 1, 2, 3, or 4 independently selected R^{5A} substituents;
each R^{w3} is independently selected from H, C₁₋₆ alkyl, C₁₋₆ haloalkyl, C₂₋₆ alkenyl, and C₃₋₆ alkynyl;
each R^{w4} is independently selected from C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl;
R^{w5} is C₁₋₆ alkyl which is substituted with CN or OR^{a51};
each R^{5A} is independently selected from halo, oxo, C₁₋₆ alkyl, C₁₋₆ haloalkyl, C₂₋₆ alkenyl, C₂₋₆ alkynyl, CN, OR^{a51}, and NR^{c51}R^{d51}, wherein the C₁₋₆ alkyl, C₂₋₆ alkenyl, and C₂₋₆ alkynyl of R^{5A} are each optionally substituted with OR^{a52} and 1, 2, 3, 4, 5, 6, or 7 independently selected halo groups;
each R^{a51}, R^{c51}, and R^{d51} is independently selected from H, C₁₋₆ alkyl, C₁₋₆ haloalkyl, C₂₋₆ alkenyl, and C₂₋₆ alkynyl; and
each R^{a52} is independently selected from H and C₁₋₆ alkyl.

20. The compound of claim 1, or a pharmaceutically acceptable salt thereof, wherein each R⁴ is independently selected from halo, C₁₋₆ alkyl, C₆₋₁₀ aryl, 5-6 membered heteroaryl, 4-10 membered heterocycloalkyl, (4-10 membered heterocycloalkyl)-C₁₋₆ alkyl-, OR^{a4}, and C(O)R^{b4}, wherein the C₁₋₆ alkyl, C₆₋₁₀ aryl, 5-6 membered heteroaryl, 4-10 membered heterocycloalkyl, and (4-10 membered heterocycloalkyl)-C₁₋₆ alkyl- of R⁴ are each optionally substituted with 1, 2, 3, or 4 independently selected R^{4A} substituents; and
each R^{a4} and R^{b4} is independently selected from H, C₁₋₆ alkyl, C₁₋₆ haloalkyl, and 4-6 membered heterocycloalkyl.

21. The compound of claim 1, or a pharmaceutically acceptable salt thereof, wherein each R⁴ is independently selected from halo, C₁₋₆ alkyl, phenyl, 5-6 membered heteroaryl, 4-10 membered heterocycloalkyl, (4-6 membered heterocycloalkyl)-C₁₋₆ alkyl-, OR^{a4}, and C(O)R^{b4}, wherein the C₁₋₆ alkyl, phenyl, 5-6 membered heteroaryl, 4-10 membered heterocycloalkyl, and (4-6 membered heterocycloalkyl)-C₁₋₆ alkyl- of R⁴ are each optionally substituted with 1, 2, 3, or 4 independently selected R^{4A} substituents; and
each R^{a4} and R^{b4} is independently selected from C₁₋₆ alkyl and 4-6 membered heterocycloalkyl.

22. The compound of claim 1, or a pharmaceutically acceptable salt thereof, wherein each R⁴ is independently selected from fluoro, methyl, trideuteromethyl, isopropyl, methoxy, morpholinylcarbonyl, phenyl, pyrazolyl, pyridinyl, tetrahydropyranyl, piperidinyl, and 2,3-dihydrobenzo[b][1,4]dioxinyl, wherein the methyl, isopropyl, phenyl, pyrazolyl, piperidinyl, and 2,3-dihydrobenzo[b][1,4]dioxinyl of R⁴ are each optionally substituted with 1, 2, 3, or 4 independently selected R^{4A} substituents.

23. The compound of claim 1, or a pharmaceutically acceptable salt thereof, wherein each R^{4A} is independently selected from C₁₋₆ alkyl, C₁₋₆ haloalkyl, CN, OR^{a41}, and C(O)R^{b41}; and
each R^{a41} and R^{b41} is independently selected from H and C₁₋₆ alkyl.

24. The compound of claim 1, or a pharmaceutically acceptable salt thereof, wherein each R^{4A} is selected from methyl, difluoromethyl, trifluoromethyl, CN, OH, and methylcarbonyl.

25. The compound of claim 1, or a pharmaceutically acceptable salt thereof, wherein each R⁴ is independently selected from fluoro, methyl, hydroxymethyl, hydroxyisopropyl, methoxy, (methylcarbonyl)piperidinyl, methylpyrazolyl, cyanophenyl, 2,3-dihydrobenzo[b][1,4]dioxinyl, (difluoromethylazetidinyl)methyl, and morpholinylcarbonyl.

26. The compound of claim 1, or a pharmaceutically acceptable salt thereof, wherein R⁵ is selected from C₁₋₆ alkyl, C₃₋₇ cycloalkyl, phenyl, 5-10 membered heteroaryl, tetrahydropyranyl, pyrrolidinyl, piperidinyl, 2,3-dihydrobenzo[b][1,4]dioxinyl, 3,4-dihydro-2H-benzo[b][1,4]oxazinyl, azaspiro[2,4]heptanyl, OR^{w2}, NHR^{w2}, C(O)R^{w2}, and C(O)NHR^{w2};
wherein the C₁₋₆ alkyl and phenyl of R⁵ are each substituted with 1 R^{w1} substituent and optionally substituted 1 or 2 independently selected R^{5A} substituents; or
the phenyl of R⁵ is substituted with 1 R^{w5} substituent and optionally substituted 1 or 2 independently selected R^{5A} substituents; and
the C₃₋₇ cycloalkyl, 5-10 membered heteroaryl, tetrahydropyranyl, pyrrolidinyl, piperidinyl, 2,3-dihydrobenzo[b][1,4]dioxinyl, 3,4-dihydro-2H-benzo[b][1,4]oxaziny], and azaspiro[2,4]heptanyl of R⁵ are each optionally substituted with 1, 2, 3, or 4 independently selected R^{5A} substituents.

27. The compound of claim 1, or a pharmaceutically acceptable salt thereof, wherein R⁵ is selected from —CH—R^{w1}, C₃₋₇ cycloalkyl, -phenyl-R^{w1}, -phenyl-R^{w5}, 5-10 membered heteroaryl, tetrahydropyranyl, pyrrolidinyl, piperidinyl, 2,3-dihydrobenzo[b][1,4]dioxinyl, 3,4-dihydro-2H-benzo[b][1,4]oxazinyl, azaspiro[2,4]heptanyl, OR^{w2}, NHR^{w2}, C(O)R^{w2}, and C(O)NHR^{w2}, wherein the C₃₋₇ cycloalkyl, phenyl, 5-10 membered heteroaryl, pyrrolidinyl, piperidinyl, 2,3-dihydrobenzo[b][1,4]dioxinyl, 3,4-dihydro-2H-benzo[b][1,4]oxazinyl, and azaspiro[2,4]heptanyl of R⁵ are each optionally substituted with 1, 2, 3, or 4 independently selected R^{5A} substituents.

28. The compound of claim 1, or a pharmaceutically acceptable salt thereof, wherein R^{w1} is selected from C₃₋₇ cycloalkyl, 4-7 membered heterocycloalkyl, OR^{w4}, and NHR^{w4}, wherein the C₃₋₇ cycloalkyl and 4-7 membered heterocycloalkyl or R^{w1} are each optionally substituted by 1 or 2 independently selected R^{5A} substituents.

29. The compound of claim 1, or a pharmaceutically acceptable salt thereof, wherein R^{w1} is selected from cyclopropyl, fluoropyrrolidinyl, morpholinyl, OR^{w4}, and NHR^{w4}; and
R^{w4} is selected from C₁₋₆ haloalkyl and C₃₋₇cycloalkyl.

30. The compound of claim 1, or a pharmaceutically acceptable salt thereof, wherein R^{w1} is selected from cyclopropyl, fluoropyrrolidinyl, morpholinyl, trifluoromethoxy, and cyclopentylamino.

31. The compound of claim 1, or a pharmaceutically acceptable salt thereof, wherein R^{w2} is selected from C₃₋₇ cycloalkyl, bicyclic 8-12 membered heteroaryl, monocyclic 4-7 membered heterocycloalkyl, and bicyclic 8-12 membered heterocycloalkyl, wherein the C₃₋₇ cycloalkyl, bicyclic 8-12 membered heteroaryl, monocyclic 4-7 membered heterocycloalkyl, and bicyclic 8-12 membered heterocycloalkyl of R^{w2} are each optionally substituted with 1, 2, 3, or 4 independently selected R^{5A} substituents.

32. The compound of claim 1, or a pharmaceutically acceptable salt thereof, wherein R^{w2} is selected from cyclobutyl, cyclopentyl, cyclohexyl, quinolinyl, tetrahydropyranyl, piperidinyl, morpholinyl, pyrrolidinyl, and octahydro-2H-pyrido[1,2-a]pyrazinyl, wherein the cyclobutyl, cyclopentyl, cyclohexyl, quinolinyl, tetrahydropyranyl, piperidinyl, morpholinyl, pyrrolidinyl, and octahydro-2H-pyrido[1,2-a]pyrazinyl of R^{w2} are each optionally substituted with 1, 2, 3, or 4 independently selected R^{5A} substituents.

33. The compound of claim 1, or a pharmaceutically acceptable salt thereof, wherein R^{w5} is C₁₋₆ alkyl which is substituted with CN or OR^{a51}; and
R^{a51} is selected from H and C₁₋₃ alkyl.

34. The compound of claim 1, or a pharmaceutically acceptable salt thereof, wherein R^{w5} is selected from cyanomethyl and hydroxymethyl.

35. The compound of claim 1, or a pharmaceutically acceptable salt thereof, wherein each R^{5A} is independently selected from halo, C₁₋₆ alkyl, C₁₋₆ haloalkyl, CN, OR^{a51}, and NR^{c51}R^{d51}, wherein the C₁₋₆ alkyl is optionally substituted with OH and 1, 2, 3, 4, 5, 6, or 7 independently selected halo groups; and
each R^{a51}, R^{c51}, and R^{d51} is independently selected from H and C₁₋₆ alkyl.

36. The compound of claim 1, or a pharmaceutically acceptable salt thereof, wherein each R^{5A} is independently selected from chloro, fluoro, methyl, hydroxyisopropyl, hydroxyhexafluoroisopropyl, difluoromethyl, trifluoromethyl, CN, methoxy, ethoxy, and amino.

37. The compound of claim 1, or a pharmaceutically acceptable salt thereof, wherein Cy⁴ is selected from quinolinyl, imidazo[1,2-b]pyridazinyl, and pyrazolo[1,5-a]pyrimidinyl, wherein the quinolinyl, imidazo[1,2-b]pyridazinyl, and pyrazolo[1,5-a]pyrimidinyl are each optionally substituted with 1, 2, 3, or 4 independently selected R⁴ substituents.

38. The compound of claim 1, wherein the compound of Formula I is a compound of Formula IVa, IVb, IVc, IVd, IVe, IVf, IVg, IVh, IVi, IVj, IVk, IVm, IVn, IVo, IVp, IVq, IVr, IVs, IVt, IVu, or IVv: 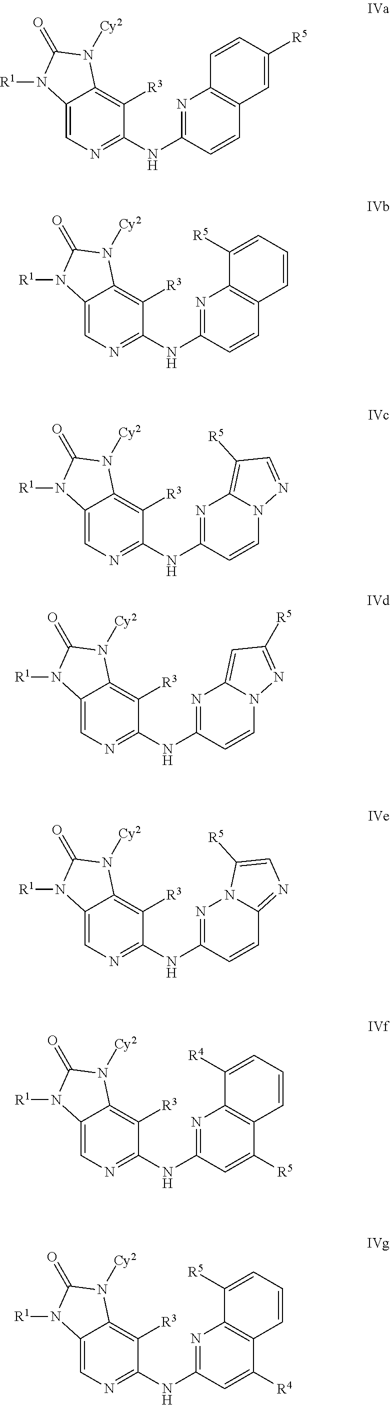 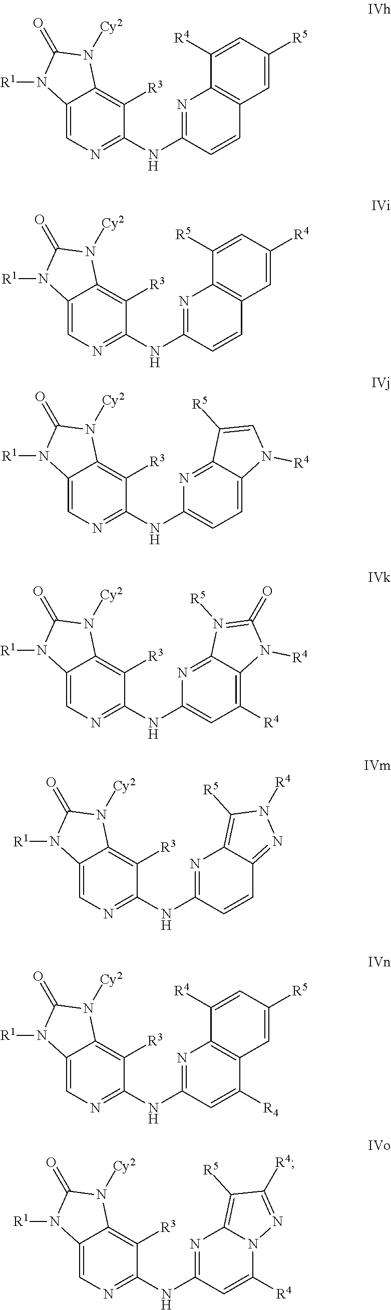 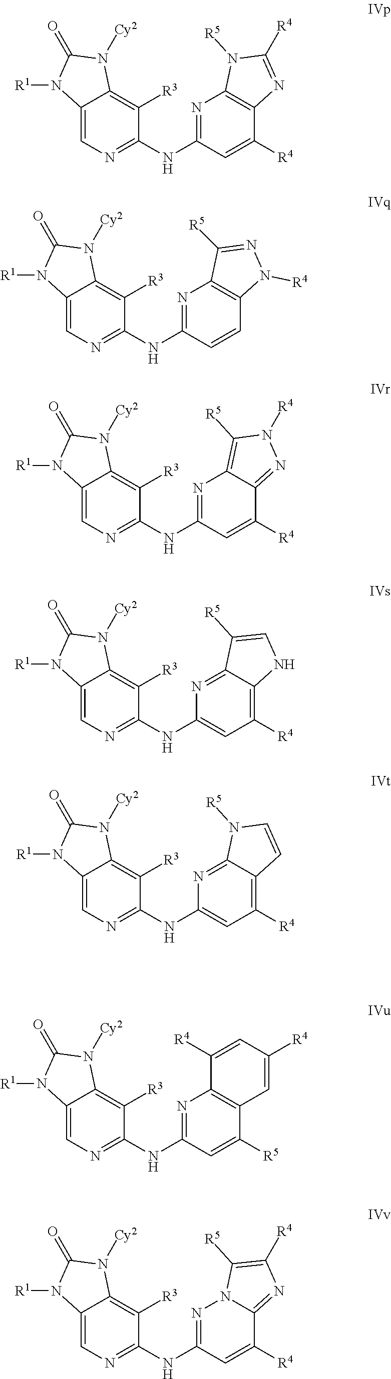
or a pharmaceutically acceptable salt thereof.

39. The compound of claim 1, or a pharmaceutically acceptable salt thereof, wherein:
each R⁴ is independently selected from halo, C₁₋₆ alkyl, C₁₋₆ haloalkyl, C₂₋₆ alkenyl, C₂₋₆ alkynyl, C₃₋₇ cycloalkyl, 4-7 membered heterocycloalkyl, 5-6 membered heteroaryl, and C₃₋₇ cycloalkyl-C₁₋₆ alkyl-, wherein each C₁₋₆ alkyl, C₂₋₆ alkenyl, C₂₋₆ alkynyl, C₃₋₇ cycloalkyl, 4-7 membered heterocycloalkyl, 5-6 membered heteroaryl, and C₃₋₇cycloalkyl-C₁₋₆ alkyl- of R⁴ is optionally substituted with 1, 2, 3, or 4 independently selected R^{4A} substituents;
each R^{4A} is independently selected from halo, C₁₋₆ alkyl, C₁₋₆ haloalkyl, C₂₋₆ alkenyl, C₂₋₆ alkynyl, CN, and OR^{w41};
each R^{a41} is independently selected from H, C₁₋₆ alkyl, C₁₋₆ haloalkyl, C₂₋₆ alkenyl, and C₂₋₆ alkynyl;
R⁵ is selected from C₃₋₁₂ cycloalkyl, 5-12 membered heteroaryl, NR^{w2}R^{w3}, and C(O)R^{w2}, wherein the C₃₋₁₂ cycloalkyl and 5-12 membered heteroaryl of R⁵ are each optionally substituted with 1, 2, 3, or 4 independently selected R^{5A} substituents;
each R^{w2} is independently selected from C₃₋₁₂ cycloalkyl and 4-12 membered heterocycloalkyl, wherein the C₃₋₁₂ cycloalkyl and 4-12 membered heterocycloalkyl of R^{w2} are each optionally substituted with 1, 2, 3, or 4 independently selected R^{5A} substituents;
each R^{w3} is independently selected from H, C₁₋₆ alkyl, C₁₋₆ haloalkyl, C₂₋₆ alkenyl, or C₂₋₆ alkynyl;
each R^{5A} is independently selected from halo, C₁₋₆ alkyl, C₁₋₆ haloalkyl, C₂₋₆ alkenyl, C₂₋₆ alkynyl, CN and OR^{a51}; and
each R^{a51} is independently selected from H, C₁₋₆ alkyl, C₁₋₆ haloalkyl, C₂₋₆ alkenyl, and C₂₋₆ alkynyl.

40. The compound of claim 1, or a pharmaceutically acceptable salt thereof, wherein R⁴ is independently selected from halo, C₁₋₆ alkyl, C₁₋₆ haloalkyl, C₃₋₇ cycloalkyl, 4-7 membered heterocycloalkyl, 5-6 membered heteroaryl, and C₃₋₇cycloalkyl-C₁₋₆ alkyl-, wherein each C₁₋₆ alkyl, C₃₋₇cycloalkyl, 4-7 membered heterocycloalkyl, 5-6 membered heteroaryl, and C₃₋₇ cycloalkyl-C₁₋₆ alkyl- of R⁴ is optionally substituted with 1, 2, 3, or 4 independently selected R^{4A} substituents.

41. The compound of claim 1, or a pharmaceutically acceptable salt thereof, wherein R⁴ is independently selected from C₁₋₆ alkyl, C₁₋₆ haloalkyl, C₃₋₇ cycloalkyl, 4-7 membered heterocycloalkyl, 5-6 membered heteroaryl, and C₃₋₇ cycloalkyl-C₁₋₆ alkyl-, wherein each C₁₋₆ alkyl, C₃₋₇cycloalkyl, 4-7 membered heterocycloalkyl, 5-6 membered heteroaryl, and C₃₋₇cycloalkyl-C₁₋₆ alkyl- of R⁵ is optionally substituted with 1, 2, 3, or 4 R^{4A} substituents;
each R^{4A} is independently selected from C₁₋₆ haloalkyl, CN, and OR^{a41}; and
each R^{a41} is independently selected from H and C₁₋₆ alkyl.

42. The compound of claim 1, or a pharmaceutically acceptable salt thereof, wherein R⁴ is selected from methyl, ethyl, isopropyl, trifluoromethyl, cyclopropyl, cyclobutyl, cyclohexyl, tetrahydropyranyl, deuterotetrahydropyranyl, pyridinyl, and cyclopropylmethyl, wherein each methyl, ethyl, isopropyl, isobutyl, cyclopropyl, cyclobutyl, cyclohexyl, tetrahydropyranyl, deuterotetrahydropyranyl, pyridinyl, and cyclopropylmethyl of R⁴ is optionally substituted with 1 or 2 R^{4A} substituents independently selected from C₁₋₆ haloalkyl, CN, OH, and C₁₋₃ alkoxy.

43. The compound of claim 1, or a pharmaceutically acceptable salt thereof, wherein R⁵ is selected from C₃₋₇ cycloalkyl, 5-6 membered heteroaryl, NHR^{w2}, and C(O)R^{w2}, wherein the C₃₋₇ cycloalkyl and 5-6 membered heteroaryl of R⁵ are each optionally substituted with 1, 2, 3, or 4 independently selected R^{5A} substituents.

44. The compound of claim 1, or a pharmaceutically acceptable salt thereof, wherein R⁵ is selected from cyclopropyl, cyclobutyl, cyclohexyl, pyrazolyl, imidazolyl, pyridinyl, NHR^{w2}, and C(O)R^{w2}, wherein the cyclopropyl, cyclobutyl, cyclohexyl, pyrazolyl, imidazolyl, and pyridinyl of R⁵ are each optionally substituted with 1, 2, 3, or 4 independently selected R^{5A} substituents.

45. The compound of claim 1, or a pharmaceutically acceptable salt thereof, wherein R^{w2} is selected from C₃₋₇ cycloalkyl, 4-8 membered monocyclic heterocycloalkyl, and 6-10 membered bicyclic heterocycloalkyl, wherein the C₃₋₇ cycloalkyl, 4-8 membered monocyclic heterocycloalkyl, and 6-10 membered bicyclic heterocycloalkyl of R^{w2} are each optionally substituted with 1, 2, 3, or 4 independently selected R^{5A} substituents.

46. The compound of claim 1, or a pharmaceutically acceptable salt thereof, wherein R^{w2} is selected from cyclopentyl, piperidinyl, morpholinyl, 1,6-diazaspiro[3.3]heptanyl, 3-azabicyclo[3.1.0]hexanyl, 2-oxa-5-azabicyclo[2.2.1]heptanyl, and 2-oxa-5-azabicyclo[4.1.0]heptanyl, wherein the cyclopentyl, piperidinyl, morpholinyl, 1,6-diazaspiro[3.3]heptanyl, 3-azabicyclo[3.1.0]hexanyl, 2-oxa-5-azabicyclo[2.2.1]heptanyl, and 2-oxa-5-azabicyclo[4.1.0]heptanyl of R^{w2} are each optionally substituted with 1, 2, 3, or 4 independently selected R^{5A} substituents.

47. The compound of claim 1, or a pharmaceutically acceptable salt thereof, wherein each R^{5A} is independently selected from halo, C₁₋₆ alkyl, C₁₋₆ haloalkyl, CN, and OR^{a51}; and
each R^{a51} is independently selected from H and C₁₋₆ alkyl.

48. The compound of claim 1, or a pharmaceutically acceptable salt thereof, wherein each R^{5A} is independently selected from fluoro, methyl, trideuteromethyl, difluoromethyl, trifluoromethyl, CN, and hydroxy.

49. The compound of claim 1, or a pharmaceutically acceptable salt thereof, wherein R¹ is C₁₋₆ alkyl, which is optionally substituted with 1, 2, or 3 independently selected R^{1A} substituents.

50. The compound of claim 1, or a pharmaceutically acceptable salt thereof, wherein R¹ is C₁₋₆ alkyl.

51. The compound of claim 1, or a pharmaceutically acceptable salt thereof, wherein R¹ is methyl or trideuteromethyl.

52. The compound of claim 1, or a pharmaceutically acceptable salt thereof, wherein Cy² is selected from monocyclic C₃₋₁₀ cycloalkyl, bicyclic C₆₋₁₀ cycloalkyl, spriocyclic C₆₋₁₀ cycloalkyl, monocyclic 4-7 membered heterocycloalkyl, spriocyclic 6-10 membered heterocycloalkyl, and bicyclic 5-10 membered heterocycloalkyl, wherein the monocyclic Cate cycloalkyl, bicyclic C₆₋₁₀ cycloalkyl, spriocyclic C₆₋₁₀ cycloalkyl, monocyclic 4-7 membered heterocycloalkyl, spriocyclic 6-10 membered heterocycloalkyl, and bicyclic 5-10 membered heterocycloalkyl are each substituted with 1, 2, 3, or 4 independently selected R² substituents.

53. The compound of claim 1, or a pharmaceutically acceptable salt thereof, wherein Cy² is selected from cyclobutyl, cyclopentyl, deuterocyclopentyl, cyclohexyl, tetrahydrofuranyl, bicyclo[2.2.1]heptanyl, bicyclo[2.2.2]octanyl, spiro[3.3]heptanyl, 3-azabicyclo[3.1.0]hexanyl, 2-azaspiro[3.3]heptanyl, and 2-azabicyclo[2.2.1]heptanyl, wherein the cyclobutyl, cyclopentyl, deuterocyclopentyl, cyclohexyl, tetrahydrofuranyl, bicyclo[2.2.1]heptanyl, bicyclo[2.2.2]octanyl, spiro[3.3]heptanyl, 3-azabicyclo[3.1.0]hexanyl, 2-azaspiro[3.3]heptanyl, and 2-azabicyclo[2.2.1]heptanyl of Cy² are each optionally substituted by 1, 2, 3 or 4 independently selected R³ substituents.

54. The compound of claim 1, or a pharmaceutically acceptable salt thereof, wherein Cy² is selected from: 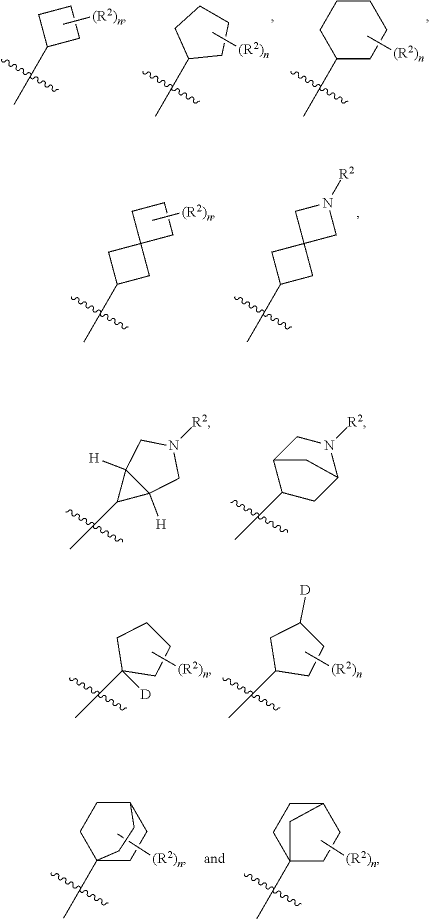
wherein n is 0, 1, or 2.

55. The compound of claim 1, or a pharmaceuticals acceptable salt thereof, wherein Cy² is selected from:

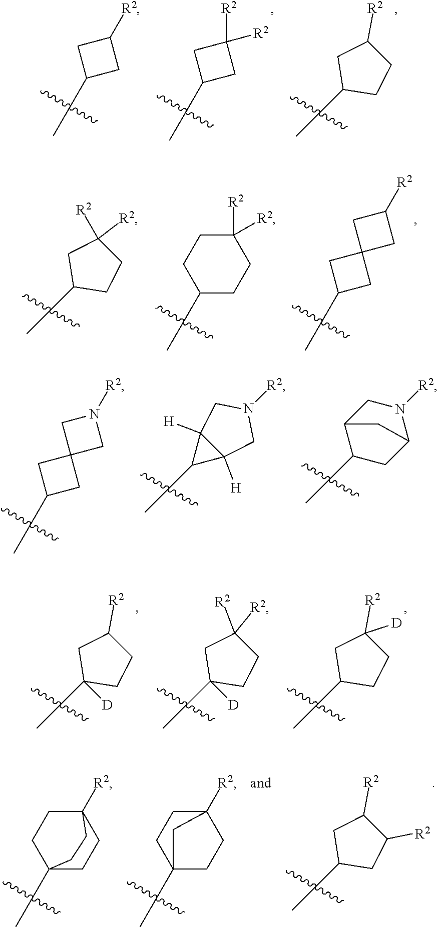

56. The compound of claim 1, or a pharmaceutically acceptable salt thereof, wherein Cy² is selected from:

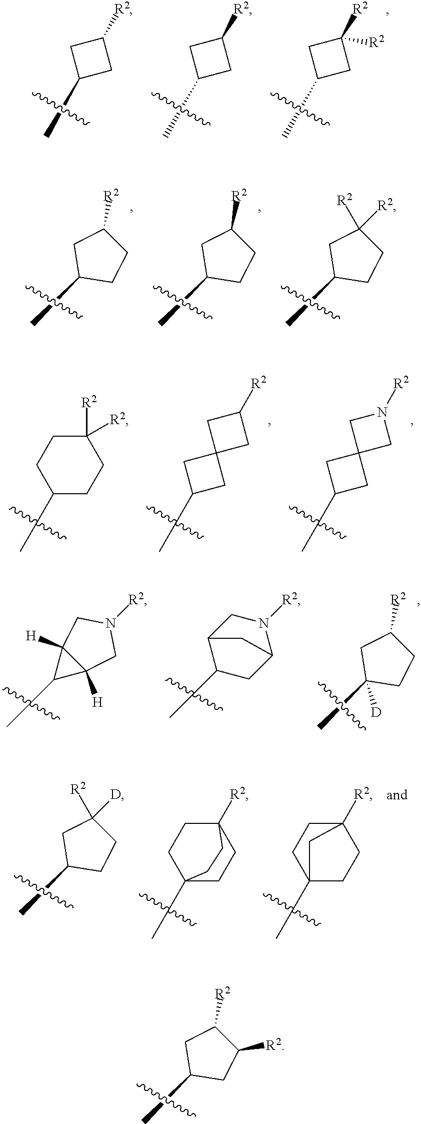

57. The compound of claim 1, or a pharmaceutically acceptable salt thereof, wherein each R′ is independently selected from halo, C₁₋₆ alkyl, C₁₋₆ haloalkyl, C₂₋₆ alkenyl, C₂₋₆ alkynyl, CN, OR^{a2}, C(O)R^{a2}, C(O)NR^{c2}R^{d2}, C(O)OR^{a2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, S(O)R^{b2}, S(O)NR^{c2}R^{d2}, S(O)₂R^{b2}, and S(O)₂NR^{c2}R^{d2}, wherein the C₁₋₆ alkyl, C₂₋₆ alkenyl, C₂₋₆ alkynyl, of R² are each optionally substituted with 1, 2, 3, or 4 independently selected R^{2A} substituents.

58. The compound of claim 1, or a pharmaceutically acceptable salt thereof, wherein each R² is independently selected from halo, C₁₋₆ alkyl, CN, OR^{a2}, C(O)R^{b2}, C(O)NR^{c2}R^{d2}, C(O)OR^{a2}, NR^{c2}C(O)R^{b2}, NR^{c2}C(O)OR^{a2}, and NR^{c2}S(O)₂R^{b2}, wherein the C₁₋₆ alkyl of R² are each optionally substituted with 1, 2, 3, or 4 independently selected R^{2A} substituents.

59. The compound of claim 1, or a pharmaceutically acceptable salt thereof, wherein each R^{a2}, R^{b2}, R^{c2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl of R^{a2}, R^{b1}, R^{c2}, and R^{d2} are each optionally substituted with 1, 2, 3, or 4 independently selected R^{2A} substituents.

60. The compound of claim 1, or a pharmaceutically acceptable salt thereof, wherein each R^{a2}, R^{b2}, R^{c2}, and R^{d2} is independently selected from H, C₁₋₆ alkyl, C₂₋₆ alkynyl, phenyl, C₃₋₇cycloalkyl, 5-6 membered heteroaryl, and 4-7 membered heterocycloalkyl, wherein the C₁₋₆ alkyl, phenyl, C₃₋₇ cycloalkyl, 5-6 membered heteroaryl, and 4-7 membered heterocycloalkyl of R^{a2}, R^{b1}, R^{c2}, and R^{d2} are each optionally substituted with 1, 2, 3, or 4 independently selected R^{2A} substituents.

61. The compound of claim 1, or a pharmaceutically acceptable salt thereof, wherein each R^{a2}, R^{b2}, R^{c2}, and R^{d2} is independently selected from H, methyl, trideuteromethyl, ethyl, propynyl, cyclopropyl, cyclobutyl, phenyl, and tetrahydropyranyl, wherein the methyl, ethyl, propynyl, cyclopropyl, cyclobutyl, phenyl, and tetrahydropyranyl of R^{a2}, R^{b1}, R^{c2}, and R^{d2} are each optionally substituted with 1, 2, 3, or 4 independently selected R^{2A} substituents.

62. The compound of claim 1, or a pharmaceutically acceptable salt thereof, wherein each R² is independently selected from halo, C₁₋₆ alkyl, CN, OR^{a2}, C(O)R^{b2}, C(O)NR^{c2}R^{d2}, C(O)OR^{a2}, NR^{c2}C(O)R^{b2}, NR^{c2}C(O)OR^{a2}, and NR^{c2}S(O)₂R^{b2}, wherein the C₁₋₆ alkyl of R³ are each optionally substituted with 1, 2, 3, or 4 independently selected R^{2A} substituents; and
each R^{a2}, R^{b2}, R^{c2}, and R^{d2} is independently selected from H, methyl, ethyl, propynyl, cyclopropyl, cyclobutyl, phenyl, and tetrahydropyranyl, wherein the methyl, trideuteromethyl, ethyl, propynyl, cyclopropyl, cyclobutyl, phenyl, and tetrahydropyranyl of R^{a2}, R^{b1}, R^{c2}, and R^{d2} are each optionally substituted with 1 or 2 independently selected R^{2A} substituents.

63. The compound of claim 1, or a pharmaceutically acceptable salt thereof, wherein each R^{2A} is independently selected from halo, C₁₋₆ alkyl, C₁₋₆ haloalkyl, C₂₋₆ alkenyl, C₂₋₆ alkynyl, CN, and OR^{a21}.

64. The compound of claim 1, or a pharmaceutically acceptable salt thereof, wherein each R^{2A} is independently selected from halo and OR^{a21}; and
each R^{a21} is independently selected from H and C₁₋₆ alkyl.

65. The compound of claim 1, or a pharmaceutically acceptable salt thereof, wherein each R^{2A} is independently selected from fluoro, hydroxy, and methoxy.

66. The compound of claim 1, or a pharmaceutically acceptable salt thereof, wherein R³ is selected from H and C₁₋₆ alkyl.

67. The compound of claim 1, or a pharmaceutically acceptable salt thereof, wherein R³ is H.

68. The compound of claim 1, or a pharmaceutically acceptable salt thereof, wherein:
R¹ is selected from C₁₋₆ alkyl, C₂₋₆ alkenyl, and C₂₋₆ alkynyl;
Cy² is selected from monocyclic C₃₋₁₀ cycloalkyl, bicyclic C₆₋₁₀ cycloalkyl, spriocyclic C₆₋₁₀ cycloalkyl, monocyclic 4-7 membered heterocycloalkyl, spriocyclic 6-10 membered heterocycloalkyl, and bicyclic 5-10 membered heterocycloalkyl, wherein the monocyclic C₃₋₁₀ cycloalkyl, bicyclic C₆₋₁₀ cycloalkyl, spriocyclic C₆₋₁₀ cycloalkyl, monocyclic 4-7 membered heterocycloalkyl, spriocyclic 6-10 membered heterocycloalkyl, and bicyclic 5-10 membered heterocycloalkyl are each substituted with 1, 2, 3, or 4 independently selected R² substituents;
each R² is independently selected from halo, C₁₋₆ alkyl, C₁₋₆ haloalkyl, C₂₋₆ alkenyl, C₂₋₆ alkynyl, CN, OR^{a2}, C(O)R^{b2}, C(O)NR^{c2}R^{d2}, C(O)OR^{a2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, S(O)R^{b2}, S(O)NR^{c2}R^{d2}, S(O)₂R^{b2}, and S(O)₂NR^{c2}R^{d2}, wherein the C₁₋₆ alkyl, C₂₋₆ alkenyl, C₂₋₆ alkynyl, of R² are each optionally substituted with 1, 2, 3, or 4 independently selected R^{2A} substituents;
each R^{a2}, R^{b2}, R^{c2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl of R^{a2}, R^{b1}, R^{c2}, and R^{d2} are each optionally substituted with 1, 2, 3, or 4 independently selected R^{2A} substituents;
each R^{2A} is independently selected from halo, C₁₋₆ alkyl, C₁₋₆ haloalkyl, C₂₋₆ alkenyl, C₂₋₆ alkynyl, CN, and OR^{a21};
each R^{a11} is independently selected from H and C₁₋₆ alkyl; and
R³ is selected from H and C₁₋₆ alkyl.

69. The compound of claim 1, or a pharmaceutically acceptable salt thereof, wherein:
R¹ is selected from C₁₋₆ alkyl:
Cy² is selected from monocyclic C₃₋₁₀ cycloalkyl, bicyclic C₆₋₁₀ cycloalkyl, spriocyclic C₆₋₁₀ cycloalkyl, monocyclic 4-7 membered heterocycloalkyl, spriocyclic 6-10 membered heterocycloalkyl, and bicyclic 5-10 membered heterocycloalkyl, wherein the monocyclic C₃₋₁₀ cycloalkyl, bicyclic C₆₋₁₀ cycloalkyl, spriocyclic C₆₋₁₀ cycloalkyl, monocyclic 4-7 membered heterocycloalkyl, spriocyclic 6-10 membered heterocycloalkyl, and bicyclic 5-10 membered heterocycloalkyl are each substituted with 1, 2, 3, or 4 independently selected R² substituents;
each R² is independently selected from halo, C₁₋₆ alkyl, CN, OR^{a2}, C(O)R^{b2}, C(O)NR^{c2}R^{d2}, C(O)OR^{a2}, NR^{c2}C(O)R^{b2}, NR^{c2}C(O)OR^{a2}, and NR^{c2}S(O)₂R^{b2}, wherein the C₁₋₆ alkyl of R² are each optionally substituted with 1, 2, 3, or 4 independently selected R^{2A} substituents;
each R^{a2}, R^{b2}, R^{c2}, and R^{d2} is independently selected from H, C₁₋₆ alkyl, C₂₋₆ alkynyl, phenyl, C₃₋₇ cycloalkyl, 5-6 membered heteroaryl, and 4-7 membered heterocycloalkyl, wherein the C₁₋₆ alkyl, phenyl, C₃₋₇cycloalkyl, 5-6 membered heteroaryl, and 4-7 membered heterocycloalkyl of R^{a2}, R^{b1}, R^{c2}, and R^{d2} are each optionally substituted with 1, 2, 3, or 4 independently selected R^{2A} substituents;
each R^{2A} is independently selected from halo, C₁₋₆ alkyl, C₁₋₆ haloalkyl, C₁₋₆ alkenyl, C₂₋₆ alkynyl, CN, and OR^{a21}:
each R^{a21} is independently selected from H and C₁₋₆ alkyl; and
R³ is H.

70. The compound of claim 1, or a pharmaceutically acceptable salt thereof, wherein:
R¹ is C₁₋₃ alkyl;
Cy² is selected from cyclobutyl, cyclopentyl, deuterocyclopentyl, cyclohexyl, tetrahydrofuranyl, bicyclo[2.2.1]heptanyl, bicyclo[2.2.2]octanyl, spiro[3.3]heptanyl, 3-azabicyclo[3.1.0]hexanyl, 2-azaspiro[3.3]heptanyl, and 2-azabicyclo[2.2.1]heptanyl, wherein the cyclobutyl, cyclopentyl, cyclohexyl, tetrahydrofuranyl, bicyclo[2.2.1]heptanyl, bicyclo[2.2.2]octanyl, spiro[3.3]heptanyl, 3-azabicyclo[3.1.0]hexanyl, 2-azaspiro[3.3]heptanyl, and 2-azabicyclo[2.2.1]heptanyl of Cy² are each optionally substituted by 1, 2, 3 or 4 independently selected R² substituents;
each R² is independently selected from halo, C₁₋₆ alkyl, CN, OR^{a2}, C(O)R^{b2}, C(O)NR^{c2}R^{d2}, C(O)OR^{a2}, NR^{c2}C(O)R^{b2}, NR^{c2}C(O)OR^{a2}, and NR^{c2}S(O)₂R^{b2}, wherein the C₁₋₆ alkyl of R² are each optionally substituted with 1, 2, 3, or 4 independently selected R^{2A} substituents;
each R^{a2}, R^{b2}, R^{c2}, and R^{d2} is independently selected from H, methyl, ethyl, propynyl, cyclopropyl, cyclobutyl, phenyl, and tetrahydropyranyl, wherein the methyl, trideuteromethyl, ethyl, propynyl, cyclopropyl, cyclobutyl, phenyl, and tetrahydropyranyl of R^{a2}, R^{b1}, R^{c2}, and R^{d2} are each optionally substituted with 1, 2, 3, or 4 independently selected R^{2A} substituents;
each R^{2A} is independently selected from halo and OR^{a21};
each R^{a21} is independently selected from H and C₁₋₆ alkyl; and
R³ is H.

71. The compound of claim 1, which is selected from:
ethyl 2-((3′-cyano-5-((1-((1R,3R)-3-((methoxycarbonyl)amino)cyclopentyl)-3-methyl-2-oxo-2,3-dihydro-1H-imidazo[4,5-c]pyridin-6-yl)amino)-[1,1′-bipheny]]-3-yl)amino)oxazole-5-carboxylate;
methyl ((1R,3R)-3-(6-((3′-cyano-5-(4-fluorotetrahydro-2H-pyran-4-carboxamido)-[1, l′-biphenyl]-3-yl)amino)-3-methyl-2-oxo-2,3-dihydro-1H-imidazo[4,5-c]pyridin-1-yl)cyclopentyl)carbamate;
methyl ((1R,3R)-3-(3-(methyl-(3)-6-((4-(1-methyl-1,6-diazaspiro[3.3]heptane-6-carbonyl)-8-(trifluoromethyl)quinolin-2-yl)amino)-2-oxo-2,3-dihydro-1H-imidazo[4,5-c]pyridin-1-yl)cyclopentyl)carbamate;
methyl ((1R,3R)-3-(6-((6-(4-(hydroxymethyl)-3-methylphenyl)pyridin-2-yl)amino)-3-methyl-2-oxo-2,3-dihydro-1H-imidazo[4,5-c]pyridin-1-yl)cyclopentyl)carbamate;
methyl ((1R,3R)-3-(6-((6′-methoxy-[2,3′-bipyridin]-6-yl)amino)-3-methyl-2-oxo-2,3-dihydro-1H-imidazo[4,5-c]pyridin-1-yl)cyclopentyl)carbamate;
methyl ((1R,3R)-3-(6-((4-(4-cyanopiperidine-1-carbonyl)-8-(trifluoromethyl)quinolin-2-yl)amino)-3-(methyl-(3)-2-oxo-2,3-dihydro-1H-imidazo[4,5-c]pyridin-1-yl)cyclopentyl)carbamate;
methyl ((1R,3R)-3-(3-methyl-6-((6-(3-methyl-1H-indazol-5-yl)pyridin-2-yl)amino)-2-oxo-2,3-dihydro-1H-imidazo[4,5-c]pyridin-1-yl)cyclopentyl)carbamate;
methyl ((1R,3R)-3-(6-((6-(2,3-dihydrobenzo[b][1,4]dioxin-6-yl)pyridin-2-yl)amino)-3-methyl-2-oxo-2,3-dihydro-1H-imidazo[4,5-c]pyridin-1-yl)cyclopentyl)carbamate;
methyl ((1R,3R)-3-(3-methyl-2-oxo-6-((6-(quinolin-6-yl)pyridin-2-yl)amino)-2,3-dihydro-1H-imidazo[4,5-c]pyridin-1-yl)cyclopentyl)carbamate;
methyl ((1R,3R)-3-(6-((4-(1-hydroxy-3-azabicyclo[3.1.0]hexane-3-carbonyl)-8-(trifluoromethyl)quinolin-2-yl)amino)-3-(methyl-d₃)-2-oxo-2,3-dihydro-1H-imidazo[4,5-c]pyridin-]-yl)cyclopentyl)carbamate;
methyl ((1R,3R)-3-(3-methyl-6-((6-(4-methyl-3,4-dihydro-2H-benzo[b][1,4]oxazin-7-yl)pyridin-2-yl)amino)-2-oxo-2,3-dihydro-1H-imidazo[4,5-c]pyridin-1-yl)cyclopentyl)carbamate;
methyl ((1R,3R)-3-(6-((4-(2-oxa-5-azabicyclo[2.2.1]heptane-5-carbonyl)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((4-(2-oxa-5-azabicyclo[4.1.0]heptane-5-carbonyl)-8-(trifluoromethyl)quinolin-2-yl)amino)-3-(methyl-d₃)-2-oxo-2,3-dihydro-1H-imidazo[4,5-c]pyridin-]-yl)cyclopentyl)carbamate;
methyl (3-((1-((1R,3R)-3-((methoxycarbonyl)amino)cyclopentyl)-3-methyl-2-oxo-2,3-dihydro-1H-imidazo[4,5-c]pyridin-6-yl)amino)-5-(1-methyl-1H-1,2,3-triazol-4-yl)phenyl)carbamate;
methyl (3-((1-((1R,3R)-3-((methoxycarbonyl)amino)cyclopentyl)-3-methyl-2-oxo-2,3-dihydro-1H-imidazo[4,5-c]pyridin-6-yl)amino)-5-(1-methylpiperidin-4-yl)phenyl)carbamate;
methyl ((1R,3R)-3-(6-((4-(3,3-difluoropiperidine-1-carbonyl)-8-(trifluoromethyl)quinolin-2-yl)amino)-3-(methyl-(3)-2-oxo-2,3-dihydro-1H-imidazo[4,5-c]pyridin-1-yl)cyclopentyl)carbamate;
methyl ((1R,3R)-3-(6-((3-((R)-2,2-difluorocyclopropane-1-carboxamido)-5-(tetrahydro-2H-pyran-4-yl)phenyl)amino)-3-methyl-2-oxo-2,3-dihydro-1H-imidazo[4,5-c]pyridin-1-yl)cyclopentyl)carbamate;
methyl ((1R,3R)-3-(6-((6-((3,3-difluorocyclopentyl)amino)-8-(trifluoromethyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((3-(cyclobutanecarboxamido)-5-(tetrahydro-2H-pyran-4-yl)phenyl)amino)-3-methyl-2-oxo-2,3-dihydro-1H-imidazo[4,5-c]pyridin-]-yl)cyclopentyl)carbamate;
methyl ((1R,3R)-3-(3-(methyl-(3)-6-((6-(1-methyl-1H-pyrazol-4-yl)-8-(trifluoromethyl)quinolin-2-yl)amino)-2-oxo-2,3-dihydro-1H-imidazo[4,5-c]pyridin-1-yl)cyclopentyl)carbamate;
methyl (3-((1-((1R,3R)-3-((methoxycarbonyl)amino)cyclopentyl)-3-methyl-2-oxo-2,3-dihydro-1H-imidazo[4,5-c]pyridin-6-yl)amino)-5-(tetrahydro-2H-pyran-3-yl)phenyl)carbamate;
methyl ((1R,3R)-3-(6-((6-((4-cyanocyclohexyl)oxy)pyridin-2-yl)amino)-3-methyl-2-oxo-2,3-dihydro-1H-imidazo[4,5-c]pyridin-1-yl)cyclopentyl)carbamate;
methyl ((1R,3R)-3-(3-methyl-2-oxo-6-((6-(quinolin-6-yloxy)pyridin-2-yl)amino)-2,3-dihydro-1H-imidazo[4,5-c]pyridin-1-yl)cyclopentyl)carbamate;
methyl (3-((1-((1r,3r)-3-(cyclopropanecarboxamido)cyclobutyl)-3-methyl-2-oxo-2,3-dihydro-1H-imidazo[4,5-c]pyridin-6-yl)amino)-5-(1-methyl-1H-pyrazol-4-yl)phenyl)carbamate;
methyl (3-((1-(6-((ethoxycarbonyl)amino)spiro[3.3]heptan-2-yl)-3-methyl-2-oxo-2,3-dihydro-1H-imidazo[4,5-c]pyridin-6-yl)amino)-5-(1-methyl-1H-pyrazol-4-yl)phenyl)carbamate;
methyl (3-((1-(6-(cyclopropanesulfonamido)spiro[3.3]heptan-2-yl)-3-methyl-2-oxo-2,3-dihydro-1H-imidazo[4,5-c]pyridin-6-yl)amino)-5-(1-methyl-1H-pyrazol-4-yl)phenyl)carbamate;
methyl 6-(6-((3-((methoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)-2-azaspiro[3.3]heptane-2-carboxylate;
methyl (3-((1-((1r,3r)-3-cyanocyclobutyl)-3-methyl-2-oxo-2,3-dihydro-1H-imidazo[4,5-c]pyridin-6-yl)amino)-5-(1-methyl-1H-pyrazol-4-yl)phenyl)carbamate;
methyl (3-((1-((1r,3r)-3-((methoxycarbonyl)amino)-3-methylcyclobutyl)-3-methyl-2-oxo-2,3-dihydro-1H-imidazo[4,5-c]pyridin-6-yl)amino)-5-(1-methyl-1H-pyrazol-4-yl)phenyl)carbamate;
methyl (R)-(3-((1-(3,3-difluorocyclopentyl)-3-methyl-2-oxo-2,3-dihydro-1H-imidazo[4,5-c]pyridin-6-yl)amino)-5-(1-methyl-1H-pyrazol-4-yl)phenyl)carbamate;
methyl (3-((1-((1R,3S)-3-fluorocyclopentyl)-3-methyl-2-oxo-2,3-dihydro-1H-imidazo[4,5-c]pyridin-6-yl)amino)-5-(1-methyl-1H-pyrazol-4-yl)phenyl)carbamate;
methyl ((1R,3R)-3-(3-methyl-6-((6-(1-methyl-1H-pyrazol-4-yl)quinolin-2-yl)amino)-2-oxo-2,3-dihydro-1H-imidazo[4,5-c]pyridin-1-yl)cyclopentyl)carbamate;
methyl ((1R,3R)-3-(3-methyl-6-((8-(1-methyl-1H-pyrazol-4-yl)quinolin-2-yl)amino)-2-oxo-2,3-dihydro-1H-imidazo[4,5-c]pyridin-1-yl)cyclopentyl)carbamate;
methyl ((1R,3R)-3-(3-methyl-6-((3-(1-methyl-1H-pyrazol-4-yl)imidazo[1,2-b]pyridazin-6-yl)amino)-2-oxo-2,3-dihydro-1H-imidazo[4,5-c]pyridin-1-yl)cyclopentyl)carbamate;
methyl ((1R,3R)-3-(3-methyl-6-((2-(1-methyl-1H-pyrazol-4-yl)pyrazolo[1,5-a]pyrimidin-5-yl)amino)-2-oxo-2,3-dihydro-1H-imidazo[4,5-c]pyridin-1-yl)cyclopentyl)carbamate;
methyl ((1R,3R)-3-(3-methyl-6-((3-(1-methyl-1H-pyrazol-4-yl)pyrazolo[1,5-a]pyrimidin-5-yl)amino)-2-oxo-2,3-dihydro-1H-imidazo[4,5-c]pyridin-1-yl)cyclopentyl)carbamate;
methyl ((1R,3R)-3-(3-methyl-2-oxo-6-((6-((tetrahydro-2H-pyran-4-yl)oxy)pyridin-2-yl)amino)-2,3-dihydro-1H-imidazo[4,5-c]pyridin-1-yl)cyclopentyl)carbamate;
methyl ((1R,3R)-3-(3-methyl-6-((3-(1-methyl-1H-imidazol-5-yl)imidazo[1,2-b]pyridazin-6-yl)amino)-2-oxo-2,3-dihydro-1H-imidazo[4,5-c]pyridin-1-yl)cyclopentyl)carbamate;
methyl (1R,5S)-6-(6-((3-((methoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)-3-azabicyclo[3.1.0]hexane-3-carboxylate;
methyl (3-((1-(2-acetyl-2-azabicyclo[2.2.1]heptan-5-yl)-3-methyl-2-oxo-2,3-dihydro-1H-imidazo[4,5-c]pyridin-6-yl)amino)-5-(1-methyl-1H-pyrazol-4-yl)phenyl)carbamate;
methyl ((1R,3R)-3-(6-((3-((3,3-difluorocyclobutane)-1-sulfonamido)-5-(tetrahydro-2H-pyran-4-yl)phenyl)amino)-3-methyl-2-oxo-2,3-dihydro-1H-imidazo[4,5-c]pyridin-1-yl)cyclopentyl)carbamate;
methyl (3-((1-(4-((methoxycarbonyl)amino)-4-methylcyclohexyl)-3-methyl-2-oxo-2,3-dihydro-1H-imidazo[4,5-c]pyridin-6-yl)amino)-5-(1-methyl-1H-pyrazol-4-yl)phenyl)carbamate;
2-methoxyethyl ((1R,3R)-3-(6-((3-((cyclobutoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate;
methyl ((1R,3R)-3-(6-((3-((cyclobutoxycarbonyl)amino)-5-(tetrahydro-2H-pyran-4-yl)phenyl)amino)-3-methyl-2-oxo-2,3-dihydro-1H-imidazo[4,5-c]pyridin-1-yl)cyclopentyl)carbamate;
cyclobutyl ((1R,3R)-3-(6-((3-((cyclobutoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate;
ethyl ((1R,3R)-3-(6-((3-((cyclobutoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate;
prop-2-yn-1-xl ((1R,3R)-3-(6-((3-((cyclobutoxycarbonyl)amino)-5-(1-methyl-1H-pyrazol-4-yl)phenyl)amino)-3-methyl-2-oxo-2,3-dihydro-1H-imidazo[4,5-c]pyridin-]-yl)cyclopentyl)carbamate;
tetrahydro-2H-pyran-4-yl ((1R,3R)-3-(6-((3-((cyclobutoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate;
4-fluorophenyl ((1R,3R)-3-(6-((3-((cyclobutoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate;
methyl ((1R,3R)-3-(6-((6-cyclopropylpyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((6-(3-cyanophenyl)-4-(1-methyl-1H-pyrazol-4-yl)pyridin-2-yl)amino)-3-methyl-2-oxo-2,3-dihydro-1H-imidazo[4,5-c]pyridin-1-yl)cyclopentyl)carbamate;
methyl ((1R,3R)-3-(3-(methyl-d₃)-6-((4-(morpholine-4-carbonyl)-8-(trifluoromethyl)quinolin-2-yl)amino)-2-oxo-2,3-dihydro-1H-imidazo[4,5-c]pyridin-1-yl)cyclopentyl)carbamate;
methyl ((1R,3R)-3-(6-((3-((methoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate;
methyl ((1R,3R)-3-(6-((3-cyclopropylimidazo[1,2-b]pyridazin-6-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((3-((cyclopropoxycarbonyl)amino)-5-methoxyphenyl)amino)-3-methyl-2-oxo-2,3-dihydro-1H-imidazo[4,5-c]pyridin-1-yl)cyclopentyl)carbamate;
methyl ((1R,3R)-3-(6-((3-((cyclobutoxycarbonyl)amino)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate;
methyl ((1R,3R)-3-(6-((3-(cyclopropanecarboxamido)-5-(1-methyl-1H-pyrazol-4-yl)phenyl)amino)-3-methyl-2-oxo-2,3-dihydro-1H-imidazo[4,5-c]pyridin-1-yl)cyclopentyl)carbamate;
methyl ((1R,3R)-3-(3-(methyl-(3)-6-((8-(1-methylcyclopropyl)quinolin-2-yl)amino)-2-oxo-2,3-dihydro-1H-imidazo[4,5-c]pyridin-1-yl)cyclopentyl)carbamate;
methyl ((1R,3R)-3-(6-((3-(3-fluoro-1-(methyl-d₃)-1H-pyrazol-4-yl)imidazo[1,2-b]pyridazin-6-yl)amino)-3-methyl-2-oxo-2,3-dihydro-1H-imidazo[4,5-c]pyridin-1-yl)cyclopentyl)carbamate;
methyl ((1R,3R)-3-(6-((4-(cyclobutoxymethyl)-6-(2,3-dihydrobenzo[b][1,4]dioxin-6-yl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((6-(3-aminopyrrolidin-1-yl)pyridin-2-yl)amino)-3-methyl-2-oxo-2,3-dihydro-1H-imidazo[4,5-c]pyridin-1-yl)cyclopentyl)carbamate;
methyl ((1R,3R)-3-(6-((4-((cyclopentylamino)methyl)-6-(2,3-dihydrobenzo[6][1.4]dioxin-6-yl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(3-methyl-2-oxo-6-((2-(piperidin-1-yl)pyrimidin-4-yl)amino)-2,3-dihydro-1H-imidazo[4,5-c]pyridin-1-yl)cyclopentyl)carbamate;
methyl ((1R,3R)-3-(6-((2′-methoxy-4-methyl-[2,4′-bipyridin]-6-yl)amino)-3-methyl-2-oxo-2,3-dihydro-1H-imidazo[4,5-c]pyridin-1-yl)cyclopentyl)carbamate;
methyl ((1R,3R)-3-(3-methyl-2-oxo-6-((4-(pyridin-4-yl)thiazol-2-yl)amino)-2,3-dihydro-1H-imidazo[4,5-c]pyridin-1-yl)cyclopentyl)carbamate;
methyl ((1R,3R)-3-(6-((3-fluoro-6′-(trifluoromethyl)-[2,3′-bipyridin]-6-yl)amino)-3-methyl-2-oxo-2,3-dihydro-1H-imidazo[4,5-c]pyridin-1-yl)cyclopentyl)carbamate;
methyl ((1R,3R)-3-(6-((6-(2,3-dihydrobenzo[b][1.4]dioxin-6-yl)-4-(((S)-3-fluoropyrrolidin-1-yl)methyl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((6-(4-(cyanomethyl)phenyl)-5-fluoropyridin-2-yl)amino)-3-methyl-2-oxo-2,3-dihydro-1H-imidazo[4,5-c]pyridin-1-yl)cyclopentyl)carbamate;
methyl ((1R,3R)-3-(6-((6-(4-cyclopropylphenyl)-5-fluoropyridin-2-yl)amino)-3-methyl-2-oxo-2,3-dihydro-1H-imidazo[4,5-c]pyridin-1-yl)cyclopentyl)carbamate;
methyl ((1R,3R)-3-(6-((5-fluoro-6-(4-(trifluoromethoxy)phenyl)pyridin-2-yl)amino)-3-methyl-2-oxo-2,3-dihydro-1H-imidazo[4,5-c]pyridin-1-yl)cyclopentyl)carbamate;
methyl ((1R,3R)-3-(6-((6-(2,3-dihydrobenzo[b][1,4]dioxin-6-yl)-4-(morpholinomethyl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((2-(2-methoxypyridin-4-yl)pyrimidin-4-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((6-(2,3-dihydrobenzo[b][1,4]dioxin-6-yl)-4-(octahydro-2H-pyrido[1,2-a]pyrazine-2-carbonyl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((2-(5-azaspiro[2,4]heptan-5-yl)pyrimidin-4-yl)amino)-3-methyl-2-oxo-2,3-dihydro-1H-imidazo[4,5-c]pyridin-1-yl)cyclopentyl)carbamate;
methyl ((1R,3R)-3-(6-((2-(3,3-difluoropyrrolidin-1-yl)pyrimidin-4-yl)amino)-3-methyl-2-oxo-2,3-dihydro-1H-imidazo[4,5-c]pyridin-1-yl)cyclopentyl)carbamate;
methyl ((1R,3R)-3-(3-methyl-2-oxo-6-((2-(4-(trifluoromethyl)piperidin-1-yl)pyrimidin-4-yl)amino)-2,3-dihydro-1H-imidazo[4,5-c]pyridin-1-yl)cyclopentyl)carbamate;
methyl ((1R,3R)-3-(6-((6-(2,3-dihydrobenzo[b][1,4]dioxin-6-yl)-4-((R)-3-fluoropyrrolidine-1-carbonyl)pyridin-2-yl)amino)-3-(methyl-(3)-2-oxo-2,3-dihydro-1H-imidazo[4,5-c]pyridin-1-yl)cyclopentyl)carbamate;
methyl ((1R,3R)-3-(3-methyl-2-oxo-6-((1-(pyridin-4-yl)-1H-pyrazol-3-yl)amino)-2,3-dihydro-1H-imidazo[4,5-c]pyridin-1-yl)cyclopentyl)carbamate;
methyl ((1R,3R)-3-(6-((6-(2,3-dihydrobenzo[b][1,4]dioxin-6-yl)-4-(morpholine-4-carbonyl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((2-((3,3-difluorocyclopentyl)amino)pyrimidin-4-yl)amino)-3-methyl-2-oxo-2,3-dihydro-1H-imidazo[4,5-c]pyridin-1-yl)cyclopentyl)carbamate;
methyl ((1R,3R)-3-(6-((2-(3,3-difluoropyrrolidin-1-yl)-6-methylpyrimidin-4-yl)amino)-3-methyl-2-oxo-2,3-dihydro-1H-imidazo[4,5-c]pyridin-1-yl)cyclopentyl)carbamate;
methyl ((1R,3R)-3-(3-methyl-2-oxo-6-((2-((tetrahydro-2H-pyran-4-yl)amino)pyrimidin-4-yl)amino)-2,3-dihydro-1H-imidazo[4,5-c]pyridin-1-yl)cyclopentyl)carbamate;
methyl ((1R,3R)-3-(3-methyl-2-oxo-6-((6-(piperidine-1-carbonyl)pyridin-2-yl)amino)-2,3-dihydro-1H-imidazo[4,5-c]pyridin-1-yl)cyclopentyl)carbamate; and
methyl ((1R,3R)-3-(6-((6-(cyclohexylcarbamoyl)pyridin-2-yl)amino)-3-methyl-2-oxo-2,3-dihydro-1H-imidazo[4,5-c]pyridin-1-yl)cyclopentyl)carbamate;
or a pharmaceutically acceptable salt thereof.

72. A compound, which is selected from:
cyclobutyl (3-((1-cyclopentyl-3-methyl-2-oxo-2,3-dihydro-1H-imidazo[4,5-c]pyridin-6-yl)amino)-5-(1-methyl-1H-pyrazol-4-yl)phenyl)carbamate;
cyclobutyl (3-((1-cyclopentyl-3-methyl-2-oxo-2,3-dihydro-1H-imidazo[4,5-c]pyridin-6-yl)amino)-5-(1-cyclopropyl-1H-pyrazol-4-yl)phenyl)carbamate; and
cyclobutyl (3-(1-cyclopropyl-1H-pyrazol-4-yl)-5-((3-methyl-2-oxo-]-(tetrahydrofuran-3-yl)-2,3-dihydro-1H-imidazo[4,5-c]pyridin-6-yl)amino)phenyl)carbamate;
or a pharmaceutically acceptable salt thereof.

73. The compound of claim 1, which is selected from:
methyl ((1R,3R)-3-(6-((1-(2-methoxyethyl)-3-(1-(trifluoromethyl)-1H-pyrazol-4-yl)-1H-pyrrolo[3,2-b]pyridin-5-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((3-(1-(difluoromethyl)-1H-pyrazol-4-yl)-1-methyl-2-oxo-7-(4-(trifluoromethyl)pyridin-3-yl)-2,3-dihydro-1H-imidazo[4,5-b]pyridin-5-yl)amino)-3-(methyl-d₃)-2-oxo-2,3-dihydro-1H-imidazo[4,5-c]pyridin-1-yl)cyclopentyl)carbamate;
N-(6-(3-(methyl-(3)-6-((4-(morpholine-4-carbonyl)-8-(trifluoromethyl)quinolin-2-yl)amino)-2-oxo-2,3-dihydro-1H-imidazo[4,5-c]pyridin-1-yl)spiro[3.3]heptan-2-yl)cyclopropanecarboxamide;
N-((1s,4s)-1-methyl-4-(3-(methyl-d₃)-6-((2-methyl-3-(1-(trifluoromethyl)-1H-pyrazol-4-yl)-2H-pyrazolo[4,3-b]pyridin-5-yl)amino)-2-oxo-2,3-dihydro-1H-imidazo[4,5-c]pyridin-1-yl)cyclohexyl)cyclopropanecarboxamide;
N-((1S,3R)-3-(6-((6-(5-chlorothiazol-2-yl)-4-(2-hydroxypropan-2-yl)pyridin-2-yl)amino)-3-(methyl-d₃)-2-oxo-2,3-dihydro-1H-imidazo[4,5-c]pyridin-1-yl)-1-methylcyclopentyl)cyclopropanecarboxamide;
methyl ((1R,3R)-3-(6-((6-(5-chlorothiazol-2-yl)-4-(2-hydroxypropan-2-yl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((4-(2-hydroxypropan-2-yl)-6-(1-methyl-1H-pyrazol-4-yl)-8-(trifluoromethyl)quinolin-2-yl)amino)-3-(methyl-d₃)-2-oxo-2,3-dihydro-1H-imidazo[4,5-c]pyridin-]-yl)cyclopentyl)carbamate;
methyl ((1R,3R)-3-(6-((4-(2-hydroxypropan-2-yl)-6-(2-(trifluoromethyl)thiazol-5-yl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((8-(2-cyanopropan-2-yl)-4-(morpholine-4-carbonyl)quinolin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((3-(1-(difluoromethyl)-1H-pyrazol-4-yl)-2-methyl-8-(4-(trifluoromethyl)pyridin-3-yl)imidazo[1,2-b]pyridazin-6-yl)amino)-3-(methyl-d₃)-2-oxo-2,3-dihydro-1H-imidazo[4,5-c]pyridin-1-yl)cyclopentyl-3-d)carbamate;
methyl ((1R,3R)-3-(6-((5-methoxy-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((6-(5-(2-hydroxypropan-2-yl)thiazol-2-yl)-4-methylpyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((4-((3-(difluoromethyl)azetidin-1-yl)methyl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((6-cyclobutoxy-5-(1-methyl-1H-pyrazol-4-yl)-4-(morpholine-4-carbonyl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((2-(5-(difluoromethyl)thiophen-2-yl)-6-(hydroxymethyl)pyrimidin-4-yl)amino)-3-(methyl-(3)-2-oxo-2,3-dihydro-1H-imidazo[4,5-c]pyridin-1-yl)cyclopentyl)carbamate;
methyl ((1R,3R)-3-(6-((6-(5-(difluoromethyl)thiophen-2-yl)-4-(morpholine-4-carbonyl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((4-(1-acetylpiperidin-4-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl)carbamate;
6-((3-(1-(difluoromethyl)-1H-pyrazol-4-yl)-2-methyl-8-(4-(trifluoromethyl)pyridin-3-yl)imidazo[1,2-b]pyridazin-6-yl)amino)-1-((1r,3r)-3-(2-hydroxypropan-2-yl)cyclobutyl)-3-(methyl-d₃)-1,3-dihydro-2H-imidazo[4,5-c]pyridin-2-one;
methyl ((1R,3R)-3-(6-((6-(5-(2-hydroxypropan-2-yl)thiazol-2-yl)-4-(1-methyl-1H-pyrazol-4-yl)pyridin-2-yl)amino)-3-(methyl-d₃)-2-oxo-2,3-dihydro-1H-imidazo[4,5-c]pyridin-1-yl)cyclopentyl)carbamate; and
methyl ((1R,3R)-3-(3-(methyl-d₃)-2-oxo-6-((6-(4-(trifluoromethyl)-1H-pyrazol-1-yl)pyridin-2-yl)amino)-2,3-dihydro-1H-imidazo[4,5-c]pyridin-1-yl)cyclopentyl)carbamate;
or a pharmaceutically acceptable salt thereof.

74. The compound of claim 1, which is selected from:
methyl ((1R,3R)-3-(3-(methyl-d₃)-2-oxo-6-((1-(tetrahydro-2H-pyran-4-yl)-3-(1-(trifluoromethyl)-1H-pyrazol-4-yl)-1H-pyrrolo[3,2-b]pyridin-5-yl)amino)-2,3-dihydro-1H-imidazo[4,5-c]pyridin-1-yl)cyclopentyl)carbamate;
methyl ((1R,3R)-3-(6-((1-(2-hydroxy-2-methylpropyl)-3-(1-(trifluoromethyl)-1H-pyrazol-4-yl)-1H-pyrrolo[3,2-b]pyridin-5-yl)amino)-3-(methyl-d₃)-2-oxo-2,3-dihydro-1H-imidazo[4,5-c]pyridin-1-yl)cyclopentyl)carbamate;
methyl ((3R)-3-(3-(methyl-d₃)-2-oxo-6-((1-(tetrahydro-2H-pyran-4-yl)-3-(1-(trifluoromethyl)-1H-pyrazol-4-yl)-1H-pyrrolo[3,2-b]pyridin-5-yl)amino)-2,3-dihydro-1H-imidazo[4,5-c]pyridin-1-yl)cyclopentyl-1-d)carbamate;
methyl ((3R)-3-(3-(methyl-d₃)-2-oxo-6-((1-(tetrahydro-2H-pyran-4-yl-4-d)-3-(1-(trifluoromethyl)-1H-pyrazol-4-yl)-1H-pyrrolo[3,2-b]pyridin-5-yl)amino)-2,3-dihydro-1H-imidazo[4,5-c]pyridin-1-yl)cyclopentyl-1-d)carbamate;
methyl ((3R)-3-(6-((1-(1-cyanocyclopropyl)-3-(1-(trifluoromethyl)-1H-pyrazol-4-yl)-1H-pyrrolo[3,2-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((1-((1-cyanocyclopropyl)methyl)-3-(1-(difluoromethyl)-1H-pyrazol-4-yl)-2-oxo-2,3-dihydro-1H-imidazo[4,5-b]pyridin-5-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((3-(1-(difluoromethyl)-1H-pyrazol-4-yl)-7-(2-hydroxypropan-2-yl)-2-methyl-3H-imidazo[4,5-b]pyridin-5-yl)amino)-3-(methyl-d′3)-2-oxo-2,3-dihydro-1H-imidazo[4,5-c]pyridin-1-yl)cyclopentyl)carbamate;
N-((1R,3R)-1-methyl-3-(3-(methyl-d₃)-6-((4-(morpholine-4-carbonyl)-8-(trifluoromethyl)quinolin-2-yl)amino)-2-oxo-2,3-dihydro-1H-imidazo[4,5-c]pyridin-1-yl)cyclopentyl)cyclopropanecarboxamide;
N-((1S,3R)-3-(6-((7-(hydroxymethyl)-2-methyl-3-(1-(trifluoromethyl)-1H-pyrazol-4-yl)-2H-pyrazolo[4,3-b]pyridin-5-yl)amino)-3-(methyl-d₃)-2-oxo-2,3-dihydro-1H-imidazo[4,5-c]pyridin-1-yl)-1-methylcyclopentyl)cyclopropanecarboxamide;
methyl ((1R,3R)-3-(6-((1-(2-hydroxy-2-methylpropyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)cyclopentyl)carbamate;
N-((1S,3R)-3-(6-((1-(2-hydroxy-2-methylpropyl)-3-(1-(trifluoromethyl)-1H-pyrazol-4-yl)-1H-pyrazolo[4,3-b]pyridin-5-yl)amino)-3-(methyl-(3)-2-oxo-2,3-dihydro-1H-imidazo[4,5-c]pyridin-1-yl)-1-methylcyclopentyl)cyclopropanecarboxamide;
N-((1s,4s)-4-(6-((1-(2-hydroxy-2-methylpropyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)-1-methylcyclohexyl)cyclopropanecarboxamide;
N-((1S,3R)-3-(6-((2-((1s,3s)-3-hydroxycyclobutyl)-3-(1-(trifluoromethyl)-1H-pyrazol-4-yl)-2H-pyrazolo[4,3-b]pyridin-5-yl)amino)-3-(methyl-d₃)-2-oxo-2,3-dihydro-1H-imidazo[4,5-c]pyridin-]-yl)-1-methylcyclopentyl)cyclopropanecarboxamide;
methyl ((1R,3R)-3-(6-((2-(2-hydroxy-2-methylpropyl)-3-(1-(trifluoromethyl)-1H-pyrazol-4-yl)-2H-pyrazolo[4,3-b]pyridin-5-yl)amino)-3-(methyl-d₃)-2-oxo-2,3-dihydro-1H-imidazo[4,5-c]pyridin-1-yl)cyclopentyl)carbamate;
N-((1S,3R)-3-(6-((2-(2-hydroxy-2-methylpropyl)-3-(1-(trifluoromethyl)-1H-pyrazol-4-yl)-2H-pyrazolo[4,3-b]pyridin-5-yl)amino)-3-(methyl-d₃)-2-oxo-2,3-dihydro-1H-imidazo[4,5-c]pyridin-1-yl)-1-methylcyclopentyl)cyclopropanecarboxamide;
methyl ((3R)-3-(6-((1-(2-hydroxy-2-methylpropyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-]-yl)cyclopentyl-1-d′)carbamate;
methyl ((3R)-3-(6-((1-(2-hydroxy-2-methylpropyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)cyclopentyl-1-d)carbamate;
N-((1s,4s)-4-(6-((1-((1r,4r)-4-hydroxycyclohexyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)-1-methylcyclohexyl)cyclopropanecarboxamide;
N-((1s,4s)-1-methyl-4-(3-(methyl-d₃)-6-((2-methyl-7-(morpholine-4-carbonyl)-3-(1-(trifluoromethyl)-1H-pyrazol-4-yl)-2H-pyrazolo[4,3-b]pyridin-5-yl)amino)-2-oxo-2,3-dihydro-1H-imidazo[4,5-c]pyridin-1-yl)cyclohexyl)cyclopropanecarboxamide;
methyl ((1S,3R)-3-(6-((1-(2-hydroxy-2-methylpropyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)-1-methylcyclopentyl)carbamate;
N-((1S,3R)-3-(6-((1-(2-hydroxy-2-methylpropyl)-3-(1-(trifluoromethyl)-1H-pyrazol-4-yl)-1H-pyrazolo[4,3-b]pyridin-5-yl)amino)-3-(methyl-(3)-2-oxo-2,3-dihydro-1H-imidazo[4,5-c]pyridin-1-yl)-1-methylcyclopentyl)acetamide;
methyl ((3R)-3-(6-((7-(2-hydroxypropan-2-yl)-3-(1-(trifluoromethyl)-1H-pyrazol-4-yl)-1H-pyrrolo[3,2-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((3R)-3-(6-((4-(2-hydroxypropan-2-yl)-1-(1-(trifluoromethyl)-1H-pyrazol-4-yl)-1H-pyrrolo[2,3-b]pyridin-6-yl)amino)-3-(methyl-d₃)-2-oxo-2,3-dihydro-1H-imidazo[4,5-c]pyridin-1-yl)cyclopentyl-1-d)carbamate;
methyl ((3R)-3-(3-(methyl-</3)-2-oxo-6-((1-(tetrahydro-2H-pyran-4-yl)-3-(1-(trifluoromethyl)-1H-pyrazol-4-yl)-1H-pyrazolo[4,3-b]pyridin-5-yl)amino)-2,3-dihydro-1H-imidazo[4,5-c]pyridin-1-yl)cyclopentyl-1-d)carbamate;
methyl ((3R)-3-(6-((1-((1-cyanocyclopropyl)methyl)-3-(1-(trifluoromethyl)-1H-pyrazol-4-yl)-1H-pyrazolo[4,3-b]pyridin-5-yl)amino)-3-(methyl-d₃)-2-oxo-2,3-dihydro-1H-imidazo[4,5-c]pyridin-1-yl)cyclopentyl-1-d)carbamate;
methyl ((3R)-3-(6-((2-((1-cyanocyclopropyl)methyl)-3-(1-(trifluoromethyl)-1H-pyrazol-4-yl)-2H-pyrazolo[4,3-b]pyridin-5-yl)amino)-3-(methyl-d₃)-2-oxo-2,3-dihydro-1H-imidazo[4,5-c]pyridin-]-yl)cyclopentyl-1-d′)carbamate;
methyl ((1R,3R)-3-(3-(methyl-(3)-6-((6-methyl-4-(morpholine-4-carbonyl)-8-(trifluoromethyl)quinolin-2-yl)amino)-2-oxo-2,3-dihydro-1H-imidazo[4,5-c]pyridin-1-yl)cyclopentyl)carbamate;
N-((1S,3R)-3-(6-((6-(5-chlorothiazol-2-yl)-4-(2-hydroxypropan-2-yl)pyridin-2-yl)amino)-3-(methyl-(3)-2-oxo-2,3-dihydro-1H-imidazo[4,5-c]pyridin-1-yl)-1-methylcyclopentyl)acetamide;
4-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)-N-(methyl-d₃)bicyclo[2.2.2]octane-1-carboxamide;
N-cyclopropyl-4-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)bicyclo[2.2.2]octane-1-carboxamide;
4-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)bicyclo[2.2.2]octane-1-carboxamide;
N-(4-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)bicyclo[2.2.2]octan-1-yl)acetamide;
4-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)bicyclo[2.2.1]heptane-1-carboxamide;
N-(4-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)bicyclo[2.2.1]heptan-1-yl)acetamide;
N-((1S,3R)-3-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)-1-methylcyclopentyl)propionamide;
methyl ((1R,3R)-3-(6-((4-(2-hydroxypropan-2-yl)-6-(5-(2-hydroxypropan-2-yl)thiazol-2-yl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((6-(5-(1,1,1,3,3,3-hexafluoro-2-hydroxypropan-2-yl)thiazol-2-yl)-4-(2-hydroxypropan-2-yl)pyridin-2-yl)amino)-3-(methyl-d₃)-2-oxo-2,3-dihydro-1H-imidazo[4,5-c]pyridin-1-yl)cyclopentyl)carbamate;
N-((1S,3R)-3-(6-((4-(2-hydroxypropan-2-yl)-6-(2-(trifluoromethyl)thiazol-5-yl)pyridin-2-yl)amino)-3-(methyl-d₃)-2-oxo-2,3-dihydro-1H-imidazo[4,5-c]pyridin-1-yl)-1-methylcyclopentyl)acetamide;
methyl ((1R,3R)-3-(6-((6-(5-(1,1,1,3,3,3-hexafluoro-2-hydroxypropan-2-yl)thiazol-2-yl)-4-(2-hydroxypropan-2-yl)pyridin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((6-(5-(2-hydroxypropan-2-yl)thiazol-2-yl)-4-methylpyridin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((5-fluoro-6-(5-(2-hydroxypropan-2-yl)thiazol-2-yl)-4-methylpyridin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((6-(5-(2-hydroxypropan-2-yl)thiazol-2-yl)-4-(methyl-d₃)pyridin-2-yl)amino)-3-(methyl-(3)-2-oxo-2,3-dihydro-1H-imidazo[4,5-c]pyridin-1-yl)cyclopentyl-1-d)carbamate;
N-((1S,3R)-3-(6-((4-(2-hydroxypropan-2-yl)-6-(2-isopropoxythiazol-5-yl)pyridin-2-yl)amino)-3-(methyl-d₃)-2-oxo-2,3-dihydro-1H-imidazo[4,5-c]pyridin-1-yl)-1-methylcyclopentyl)acetamide;
N-((1S,3R)-3-(6-((4-(2-hydroxypropan-2-yl)-6-(2-methoxythiazol-5-yl)pyridin-2-yl)amino)-3-(methyl-(3)-2-oxo-2,3-dihydro-1H-imidazo[4,5-c]pyridin-1-yl)-1-methylcyclopentyl)acetamide;
N-((1S,3R)-3-(6-((6-(2-ethoxythiazol-5-yl)-4-(2-hydroxypropan-2-yl)pyridin-2-yl)amino)-3-(methyl-d₃)-2-oxo-2,3-dihydro-1H-imidazo[4,5-c]pyridin-1-yl)-1-methylcyclopentyl)acetamide;
methyl ((1R,3R)-3-(6-((6′-(5-(difluoromethyl)thiophen-2-yl)-4-(trifluoromethyl)-[3,4′-bipyridin]-2′-yl)amino)-3-(methyl-d₃)-2-oxo-2,3-dihydro-1H-imidazo[4,5-c]pyridin-1-yl)cyclopentyl)carbamate;
methyl ((1S,2S,4S)-2-hydroxy-4-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-]-yl)cyclopentyl)carbamate;
methyl ((1R,3R)-3-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl-3-d)carbamate;
methyl ((1R,3R)-3-(3-(methyl-d₃)-6-((4-(1-methylpiperidin-4-yl)-6-(5-(trifluoromethyl)thiazol-2-yl)pyridin-2-yl)amino)-2-oxo-2,3-dihydro-1H-imidazo[4,5-c]pyridin-1-yl)cyclopentyl)carbamate;
methyl ((1R,3R)-3-(6-((4-(2-hydroxypropan-2-yl)-6-(5-(trifluoromethyl)thiazol-2-yl)pyridin-2-yl)amino)-3-(methyl-(3)-2-oxo-2,3-dihydro-1H-imidazo[4,5-c]pyridin-1-yl)cyclopentyl-1-d)carbamate;
methyl ((1S,3R)-3-(6-((4-(2-hydroxypropan-2-yl)-6-(5-(trifluoromethyl)thiazol-2-yl)pyridin-2-yl)amino)-3-(methyl-d₃)-2-oxo-2,3-dihydro-1H-imidazo[4,5-c]pyridin-1-yl)cyclopentyl-1-d)carbamate;
methyl ((1S,3R)-3-(6-((3-(1-(difluoromethyl)-1H-pyrazol-4-yl)-2-methyl-8-(4-(trifluoromethyl)pyridin-3-yl)imidazo[1,2-b]pyridazin-6-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((6-(5-(2-hydroxypropan-2-yl)thiazol-2-yl)-4-(tetrahydro-2H-pyran-4-yl)pyridin-2-yl)amino)-3-(methyl-d₃)-2-oxo-2,3-dihydro-1H-imidazo[4,5-c]pyridin-1-yl)cyclopentyl)carbamate;
methyl ((1R,3R)-3-(6-((6-(5-(2-hydroxypropan-2-yl)thiazol-2-yl)-4-(tetrahydro-2H-pyran-4-yl)pyridin-2-yl)amino)-3-(methyl-d₃)-2-oxo-2,3-dihydro-1H-imidazo[4,5-c]pyridin-1-yl)cyclopentyl-1-d)carbamate;
methyl ((1S,3R)-3-(6-((6-(5-(2-hydroxypropan-2-yl)thiazol-2-yl)-4-(tetrahydro-2H-pyran-4-yl)pyridin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((6′-(5-(2-hydroxypropan-2-yl)thiazol-2-yl)-4-(trifluoromethyl)-(3,4′-bipyridin]-2′-yl)amino)-3-(methyl-d₃)-2-oxo-2,3-dihydro-1H-imidazo[4,5-c]pyridin-1-yl)cyclopentyl-1-d)carbamate;
methyl ((1S,3R)-3-(6-((6′-(5-(2-hydroxypropan-2-yl)thiazol-2-yl)-4-(trifluoromethyl)-(3,4′-bipyridin]-2′-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((6-(5-(2-hydroxypropan-2-yl)thiazol-2-yl)-4-(1-methylpiperidin-4-yl)pyridin-2-yl)amino)-3-(methyl-d₃)-2-oxo-2,3-dihydro-1H-imidazo[4,5-c]pyridin-1-yl)cyclopentyl-1-d)carbamate;
methyl ((1R,3R)-3-(6-((6′-(5-(2-hydroxypropan-2-yl)thiazol-2-yl)-2-(trifluoromethyl)-[3,4′-bipyridin]-2′-yl)amino)-3-(methyl-d₃)-2-oxo-2,3-dihydro-1H-imidazo[4,5-c]pyridin-1-yl)cyclopentyl-1-d)carbamate;
N-((1S,3R)-3-(6-((3-(1-(difluoromethyl)-1H-pyrazol-4-yl)-2-methyl-8-(4-(trifluoromethyl)pyridin-3-yl)imidazo[1,2-b]pyridazin-6-yl)amino)-3-(methyl-d₃)-2-oxo-2,3-dihydro-1H-imidazo[4,5-c]pyridin-1-yl)-1-methylcyclopentyl)acetamide; and
methyl ((1R,3R)-3-(6-((3-(1-(difluoromethyl)-1H-pyrazol-4-yl)-7-(2-hydroxypropan-2-yl)-2-methyl-3H-imidazo[4,5-b]pyridin-5-yl)amino)-3-(methyl-d₃)-2-oxo-2,3-dihydro-1H-imidazo[4,5-c]pyridin-1-yl)cyclopentyl-1-d)carbamate;
or a pharmaceutically acceptable salt thereof.

75. The compound of claim 1, or a pharmaceutically acceptable salt thereof, wherein the compound is deuterated.

76. A pharmaceutical composition, comprising a compound of claim 1, or a pharmaceutically acceptable salt thereof, and a pharmaceutically acceptable carrier.

77. A method of inhibiting an activity of the V617F variant of JAK2 kinase, comprising contacting the kinase with a compound of claim 1, or a pharmaceutically acceptable salt thereof.

78. A method of treating cancer in a patient in need thereof, the method comprising administering to the patient a therapeutically effective amount of a compound of claim 1, or a pharmaceutically acceptable salt thereof.

79. The method of claim 78, wherein the cancer is selected from bladder cancer, breast cancer, cervical cancer, colorectal cancer, cancer of the small intestine, colon cancer, rectal cancer, cancer of the anus, endometrial cancer, gastric cancer, head and neck cancer, kidney cancer, liver cancer, lung cancer, ovarian cancer, prostate cancer, testicular cancer, uterine cancer, vulvar cancer, esophageal cancer, gall bladder cancer, pancreatic cancer, stomach cancer, thyroid cancer, parathyroid cancer, neuroendocrine cancer, skin cancer, and brain cancer.

80. The method of claim 78, wherein the cancer is a hematological cancer.

81. The method of claim 78, wherein the cancer is selected from leukemia, lymphoma, multiple myeloma, chronic lymphocytic lymphoma, adult T cell leukemia, acute myeloid leukemia, B-cell lymphoma, cutaneous T-cell lymphoma, acute myelogenous leukemia, Hodgkin's or non-Hodgkin's lymphoma, a myeloproliferative neoplasm), myelodysplastic syndrome, chronic eosinophilic leukemia, Waldenstrom's Macroglubulinemia, hairy cell lymphoma, chronic myelogenic lymphoma, acute lymphoblastic lymphoma, AIDS-related lymphoma, and Burkitt's lymphoma.

82. A method of treating a myeloproliferative disorder in a patient in need thereof, the method comprising administering to the patient a therapeutically effective amount of a compound of claim 1, or a pharmaceutically acceptable salt thereof.

83. The method of claim 82, wherein the myeloproliferative disorder is selected from polycythemia vera, essential thrombocythemia, myelofibrosis with myeloid metaplasia, primary myelofibrosis, post-essential thrombocythemia myelofibrosis, post polycythemia vera myelofibrosis, chronic myelogenous leukemia, chronic myelomonocytic leukemia, hypereosinophilic syndrome, and systemic mast cell disease.

84. A method of treating myelodysplastic syndrome m a patient in need thereof, the method comprising administering to the patient a therapeutically effective amount of claim 1, or a pharmaceutically acceptable salt thereof.
